(19) United States
(12) Patent Application Publication
(10) Pub. No.: US 2023/0255106 A1
(43) Pub. Date: Aug. 10, 2023

(54) ORGANIC ELECTROLUMINESCENT APPARATUS

(71) Applicant: Merck Patent GmbH, Darmstadt (DE)

(72) Inventors: Amir Hossain PARHAM, Darmstadt (DE); Christian EHRENREICH, Darmstadt (DE); Jonas Valentin KROEBER, Darmstadt (DE)

(21) Appl. No.: 17/927,758

(22) PCT Filed: May 26, 2021
(86) PCT No.: PCT/EP2021/063972

§ 371 Date: Nov. 25, 2022

(30) Foreign Application Priority Data
May 29, 2020 (EP) 20177568.1

(51) Int. Cl. H10K 85/60; H10K 85/30; C09K 11/06

(52) U.S. Cl. CPC H10K 85/6572; H10K 85/654; H10K 85/6574; H10K 85/6576; H10K 85/342; C09K 11/06; H10K 50/11

(57) ABSTRACT

The present invention relates to an organic electroluminescent device comprising a mixture comprising an electron-transporting host material and a hole-transporting host material, and to a formulation comprising a mixture of the host materials and to a mixture comprising the host materials. The electron-transporting host material corresponds to a compound of the formula (1) from the class of the fused carbazole derivatives containing an asymmetrically substituted pyrimidine or triazine unit.

The present invention relates to an organic electroluminescent device comprising a mixture comprising an electron-transporting host material and a hole-transporting host material, and to a formulation comprising a mixture of the host materials and to a mixture comprising the host materials. The electron-transporting host material corresponds to a compound of the formula (1) from the class of the fused carbazole derivatives containing an asymmetrically substituted pyrimidine or triazine unit.

The structure of organic electroluminescent devices (e.g. OLEDs—organic light-emitting diodes or OLECs—organic light-emitting electrochemical cells) in which organic semiconductors are used as functional materials has long been known. Emitting materials used here, aside from fluorescent emitters, are increasingly organometallic complexes which exhibit phosphorescence rather than fluorescence. For quantum-mechanical reasons, up to a fourfold increase in energy efficiency and power efficiency is possible using organometallic compounds as phosphorescent emitters. In general terms, however, there is still a need for improvement in OLEDs, especially also in OLEDs which exhibit triplet emission (phosphorescence), for example with regard to efficiency, operating voltage and lifetime.

The properties of organic electroluminescent devices are not only determined by the emitters used. Also of particular significance here are especially the other materials used, such as host and matrix materials, hole blocker materials, electron transport materials, hole transport materials and electron or exciton blocker materials, and among these especially the host or matrix materials. Improvements to these materials can lead to distinct improvements to electroluminescent devices.

Host materials for use in organic electronic devices are well known to the person skilled in the art. The term “matrix material” is also frequently used in the prior art when what is meant is a host material for phosphorescent emitters. This use of the term is also applicable to the present invention. In the meantime, a multitude of host materials has been developed both for fluorescent and for phosphorescent electronic devices.

A further means of improving the performance data of electronic devices, especially of organic electroluminescent devices, is to use combinations of two or more materials, especially host materials or matrix materials.

U.S. Pat. No. 6,392,250 B1 discloses the use of a mixture consisting of an electron transport material, a hole transport material and a fluorescent emitter in the emission layer of an OLED. With the aid of this mixture, it was possible to improve the lifetime of the OLED compared to the prior art.

U.S. Pat. No. 6,803,720 B1 discloses the use of a mixture comprising a phosphorescent emitter and a hole transport material and an electron transport material in the emission layer of an OLED. Both the hole transport material and the electron transport material are small organic molecules.

WO2010136109 and WO2011000455 describe indenocarbazole derivatives having electron- and hole-transporting properties that can be used in the emission layer and/or charge transport layer of electroluminescent devices.

US20100187977 describes indolocarbazole derivatives as host materials for electroluminescent devices.

WO2011088877 describes specific heterocyclic compounds that can be used in an organic light-emitting device as light-emitting compound, or as host material or hole-transporting material.

WO2015014435 and WO2015051869 describe compounds for electroluminescent devices having mutually opposite electron-conducting and hole-conducting groups.

U.S. Pat. No. 9,771,373 describes specific carbazole derivatives as host material for a light-emitting layer of an electroluminescent device that can be used together with a further host material.

KR20160046077 describes specific triazine-dibenzofuran-carbazole and triazine-dibenzothiophene-carbazole derivatives in a light-emitting layer together with a further host material and a specific emitter. The carbazole here is bonded to the dibenzofuran or dibenzothiophene unit via the nitrogen atom.

US20170117488 describes specific triazine derivatives in a light-emitting layer together with biscarbazole derivatives as a further host material.

KR20180012499 describes specific indolocarbazole derivatives in a light-emitting layer together with a further host material.

However, there is still need for improvement in the case of use of these materials or in the case of use of mixtures of the materials, especially in relation to efficiency, operating voltage and/or lifetime of the organic electroluminescent device.

The problem addressed by the present invention is therefore that of providing a combination of host materials which are suitable for use in an organic electroluminescent device, especially in a fluorescent or phosphorescent OLED, and lead to good device properties, especially with regard to an improved lifetime, and that of providing the corresponding electroluminescent device.

It has now been found that this problem is solved, and the disadvantages from the prior art are eliminated, by the combination of at least one compound of the formula (1) as first host material and at least one hole-transporting compound of the formula (2) as second host material in a light-emitting layer of an organic electroluminescent device. The use of such a material combination for production of the light-emitting layer in an organic electroluminescent device leads to very good properties of these devices, especially with regard to lifetime, especially with equal or improved efficiency and/or operating voltage. The advantages are especially also manifested in the presence of a light-emitting component in the emission layer, especially in the case of combination with emitters of the formula (IIIa) or emitters of the formulae (1) to (VI) at concentrations between 2% and 15% by weight, especially concentrations of 8% by weight and 12% by weight.

The present invention therefore first provides an organic electroluminescent device comprising an anode, a cathode and at least one organic layer, containing at least one light-emitting layer, wherein the at least one light-emitting layer contains at least one compound of the formula (1) as host material 1 and at least one compound of the formula (2) as host material 2

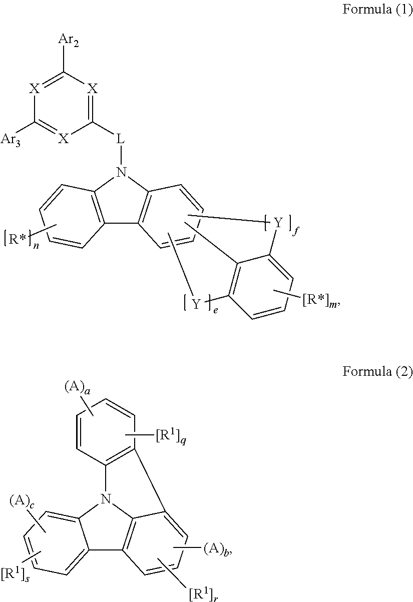

where the symbols and indices used are as follows:
X is the same or different at each instance and is CR⁰ or N, where at least two symbols X are N;
X₂ is the same or different at each instance and is CH, CR¹ or N, where not more than 2 symbols X₂ can be N;
Y is the same or different at each instance and is selected from C(R)₂ and NR;
L is the same or different at each instance and is a single bond or phenylene;
R* at each instance is independently D or an aromatic or heteroaromatic ring system that has 6 to 18 ring atoms and may be partly or fully deuterated;
R # is the same or different at each instance and is selected from the group consisting of D, F, Cl, Br, I, CN, NO₂, C(═O)R², P(═O)(Ar₁)₂, P(Ar₁)₂, B(Ar₁)₂, Si(Ar₁)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR² and where one or more hydrogen atoms may be replaced by D, F, Cl, Br, I, CN or NO₂, an aromatic or heteroaromatic ring system which has 5 to 40 ring atoms and may be substituted in each case by one or more R² radicals, an aryloxy or heteroaryloxy group which has 5 to 40 ring atoms and may be substituted by one or more R² radicals, or an aralkyl or heteroaralkyl group which has 5 to 40 ring atoms and may be substituted by one or more R² radicals;
R is the same or different at each instance and is selected from a straight-chain alkyl group having 1 to 20 carbon atoms or a branched or cyclic alkyl group having 3 to 20 carbon atoms, an aromatic or heteroaromatic ring system having 5 to 40 ring atoms, or an aralkyl or heteroaralkyl group having 5 to 40 ring atoms; at the same time, two substituents R may form a monocyclic or polycyclic, aliphatic, aromatic or heteroaromatic ring system that may be substituted by one or more R² radicals;
R¹ is the same or different at each instance and is selected from the group consisting of CN, a straight-chain alkyl, alkoxy or thioalkyl group having 1 to 20 carbon atoms or a branched or cyclic alkyl, alkoxy or thioalkyl group having 3 to 20 carbon atoms, an aromatic or heteroaromatic ring system having 5 to 40 ring atoms, an aryloxy or heteroaryloxy group having 5 to 40 ring atoms, or an aralkyl or heteroaralkyl group having 5 to 40 ring atoms; at the same time, it is possible for two substituents R¹ bonded to the same carbon atom or to adjacent carbon atoms to form a monocyclic or polycyclic, aliphatic, aromatic or heteroaromatic ring system that may be substituted by one or more R² radicals;
R⁰ and R² are the same or different at each instance and are selected from the group consisting of H, D, F, Cl, Br, I, CN, NO₂, N(Ar₁)₂, NH₂, N(R³)₂, C(═O)Ar₁, C(═O)H, C(═O)R³, P(═O)(Ar₁)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R³ radicals, where one or more nonadjacent CH₂ groups may be replaced by HC═CH, R³C═CR³, C≡C, Si(R³)₂, Ge(R³)₂, Sn(R³)₂, C═O, C═S, C═Se, C═NR³, P(═O)(R³), SO, SO₂, NH, NR³, O, S, CONH or CONR³ and where one or more hydrogen atoms may be replaced by D, F, Cl, Br, I, CN or NO₂, an aromatic or heteroaromatic ring system that has 5 to 60 ring atoms and may be substituted in each case by one or more R³ radicals, an aryloxy or heteroaryloxy group that has 5 to 60 ring atoms and may be substituted by one or more R³ radicals, or a combination of these systems, where optionally two or more adjacent substituents R² may form a monocyclic or polycyclic, aliphatic, aromatic or heteroaromatic ring system that may be substituted by one or more R³ radicals;
R³ is the same or different at each instance and is selected from the group consisting of H, D, F, CN, an aliphatic hydrocarbyl radical having 1 to 20 carbon atoms, or an aromatic or heteroaromatic ring system having 5 to 30 ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents together to form a mono- or polycyclic, aliphatic ring system;
Ar₁ is the same or different at each instance and is an aromatic or heteroaromatic ring system which has 5 to 30 ring atoms and may be substituted by one or more nonaromatic R³ radicals; at the same time, two Ar₁ radicals bonded to the same nitrogen atom, phosphorus atom or boron atom may also be bridged to one another by a single bond or a bridge selected from N(R³), C(R³)₂, O or S;
Ar₂ and Ar₃ are different at each instance;
Ar₂ at each instance is a biphenyl, a dibenzofuranyl, a dibenzothiophenyl, a carbazol-N-yl or a carbazol-N-yl-phenyl group that may be substituted by one or more R* radicals;
Ar₃ at each instance is an aryl or heteroaryl group that has 5 to 40 ring atoms and may be substituted by one or more R² radicals;
A at each instance is independently a group of the formula (3) or (4),

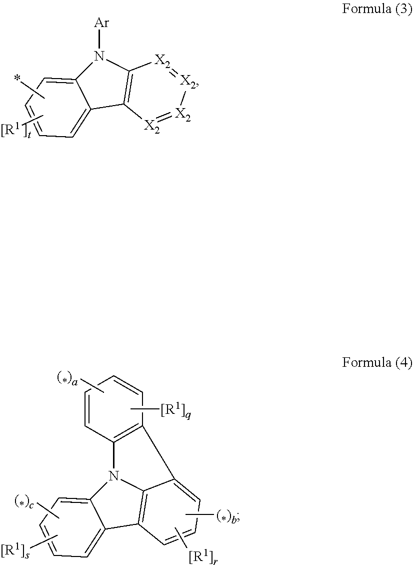

Ar at each instance is in each case independently an aryl group which has 6 to 40 ring atoms and may be substituted by one or more R # radicals, or a heteroaryl group which has 5 to 40 ring atoms and may be substituted by one or more R # radicals;
   * indicates the binding site to the formula (2);
a, b, c at each instance are each independently 0 or 1, where the sum total of the indices a+b+c at each instance is 1;
e, f at each instance are each independently 0 or 1, where the sum total of the indices e+f at each instance is 1;
n and m at each instance are independently 0, 1, 2, 3 or 4; and
q, r, s, t at each instance are each independently 0 or 1.

The invention further provides a process for producing the organic electroluminescent devices and mixtures comprising at least one compound of the formula (1) and at least one compound of the formula (2), specific material combinations and formulations that contain such mixtures or material combinations. The corresponding preferred embodiments as described hereinafter likewise form part of the subject-matter of the present invention. The surprising and advantageous effects are achieved through specific selection of the compounds of the formula (1) and the compounds of the formula (2).

The organic electroluminescent device of the invention is, for example, an organic light-emitting transistor (OLET), an organic field quench device (OFQD), an organic light-emitting electrochemical cell (OLEC, LEC, LEEC), an organic laser diode (0-laser) or an organic light-emitting diode (OLED). The organic electroluminescent device of the invention is especially an organic light-emitting diode or an organic light-emitting electrochemical cell. The device of the invention is more preferably an OLED.

The organic layer of the device of the invention that contains the light-emitting layer containing the material combination of at least one compound of the formula (1) and at least one compound of the formula (2), as described above or described hereinafter, preferably comprises, in addition to this light-emitting layer (EML), a hole injection layer (HIL), a hole transport layer (HTL), an electron transport layer (ETL), an electron injection layer (EIL) and/or a hole blocker layer (HBL). It is also possible for the device of the invention to include multiple layers from this group selected from EML, HIL, HTL, ETL, EIL and HBL.

However, the device may also comprise inorganic materials or else layers formed entirely from inorganic materials.

It is preferable that the light-emitting layer containing at least one compound of the formula (1) and at least one compound of the formula (2) is a phosphorescent layer which is characterized in that it comprises, in addition to the host material combination of the compounds of the formula (1) and formula (2), as described above, at least one phosphorescent emitter. A suitable selection of emitters and preferred emitters is described hereinafter.

An aryl group in the context of this invention contains 6 to 40 ring atoms, preferably carbon atoms. A heteroaryl group in the context of this invention contains 5 to 40 ring atoms, where the ring atoms include carbon atoms and at least one heteroatom, with the proviso that the sum total of carbon atoms and heteroatoms adds up to at least 5. The heteroatoms are preferably selected from N, O and/or S. An aryl group or heteroaryl group is understood here to mean either a simple aromatic cycle, i.e. phenyl, derived from benzene, or a simple heteroaromatic cycle, for example derived from pyridine, pyrimidine or thiophene, or a fused aryl or heteroaryl group, for example derived from naphthalene, anthracene, phenanthrene, quinoline or isoquinoline. An aryl group having 6 to 18 carbon atoms is therefore preferably phenyl, naphthyl, phenanthryl or triphenylenyl, with no restriction in the attachment of the aryl group as substituent. The aryl or heteroaryl group in the context of this invention may bear one or more R radicals, where the substituent R is described below.

An aromatic ring system in the context of this invention contains 6 to 40 ring atoms, preferably carbon atoms, in the ring system. The aromatic ring system also includes aryl groups as described above.

An aromatic ring system having 6 to 18 ring atoms is preferably selected from phenyl, biphenyl, naphthyl, phenanthryl and triphenylenyl.

A heteroaromatic ring system in the context of this invention contains 5 to 40 ring atoms and at least one heteroatom. A preferred heteroaromatic ring system has 10 to 40 ring atoms and at least one heteroatom. The heteroaromatic ring system also includes heteroaryl groups as described above. The heteroatoms in the heteroaromatic ring system are preferably selected from N, O and/or S.

An aromatic or heteroaromatic ring system in the context of this invention is understood to mean a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diaryl ethers, stilbene, etc. shall thus also be regarded as aromatic or heteroaromatic ring systems in the context of this invention, and likewise systems in which two or more aryl groups are interrupted, for example, by a linear or cyclic alkyl group or by a silyl group, for example 9,9-dialkylfluorene. In addition, systems in which two or more aryl or heteroaryl groups are bonded directly to one another, for example biphenyl, terphenyl, quaterphenyl or bipyridine, are likewise encompassed by the definition of the aromatic or heteroaromatic ring system.

An aromatic or heteroaromatic ring system which has 5-40 ring atoms and may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

The abbreviation Ar₁ is the same or different at each instance and is an aromatic or heteroaromatic ring system which has 5 to 30 ring atoms and may be substituted by one or more nonaromatic R³ radicals; at the same time, two Ar₁ radicals bonded to the same nitrogen atom, phosphorus atom or boron atom may also be bridged to one another by a single bond or a bridge selected from N(R³), C(R³)₂, O or S, where the R³ radical or the substituents R³ has/have a definition as described above or hereinafter. Preferably, Ar₁ is an aryl group having 6 to 40 carbon atoms as described above. Most preferably, Ar₁ is phenyl which may be substituted by one or more nonaromatic R³ radicals. Ar₁ is preferably unsubstituted.

The abbreviation Ar₂ at each instance is in each case independently a biphenyl, a dibenzofuranyl, a dibenzothiophenyl, a carbazol-N-yl or a carbazol-N-yl-phenyl group that may be substituted by one or more R* radicals, where the R* radical has or the substituents R* have a definition as described above or hereinafter.

The abbreviation Ar₃ at each instance is in each case independently an aryl or heteroaryl group which has 5 to 40 ring atoms and may be substituted by one or more R² radicals, where the R² radical or the substituents R² has/have a definition as described above or hereinafter. The details given for the aryl and heteroaryl groups having 5 to 40 ring atoms apply here correspondingly.

The abbreviation Ar at each instance is in each case independently an aryl group which has 6 to 40 ring atoms and may be substituted by one or more R # radicals, or a heteroaryl group which has 5 to 40 ring atoms and may be substituted by one or more R # radicals, where the details for the aryl group or heteroaryl group apply correspondingly, as described above. The R # radical or the R # radicals has/have a definition as described above or described hereinafter. The abbreviation Ar at each instance is preferably in each case independently an aryl group which has 6 to 40 carbon atoms and may be substituted by one or more R # radicals, or a heteroaryl group having 5 to 40 ring atoms and containing O or S as heteroatom, which may be substituted by one or more R # radicals, where the details for the aryl group, heteroaryl group and R # as described above or hereinafter are applicable correspondingly.

A cyclic alkyl, alkoxy or thioalkyl group in the context of this invention is understood to mean a monocyclic, bicyclic or polycyclic group.

In the context of the present invention, a straight-chain, branched or cyclic C₁- to C₂₀-alkyl group is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals.

A straight-chain or branched C₁- to C₂₀-alkoxy group is understood to mean, for example, methoxy, trifluoromethoxy, ethoxy, n-propoxy, i-propoxy, n-butoxy, i-butoxy, s-butoxy, t-butoxy or 2-methylbutoxy.

A straight-chain C₁- to C₂₀-thioalkyl group is understood to mean, for example, S-alkyl groups, for example thiomethyl, 1-thioethyl, 1-thio-i-propyl, 1-thio-n-propyl, 1-thio-i-butyl, 1-thio-n-butyl or 1-thio-t-butyl.

An aryloxy or heteroaryloxy group having 5 to 40 ring atoms means O-aryl or O-heteroaryl and means that the aryl or heteroaryl group is bonded via an oxygen atom, where the aryl or heteroaryl group is defined as described above.

An aralkyl or heteroaralkyl group having 5 to 40 ring atoms means that an alkyl group as described above is substituted by an aryl group or heteroaryl group, where the aryl or heteroaryl group is defined as described above.

A phosphorescent emitter in the context of the present invention is a compound that exhibits luminescence from an excited state with higher spin multiplicity, i.e. a spin state>1, especially from an excited triplet state. In the context of this application, all luminescent complexes with transition metals or lanthanides are to be regarded as phosphorescent emitters. A more exact definition is given hereinafter.

When the host materials of the light-emitting layer comprising at least one compound of the formula (1) as described above or described as preferred hereinafter and at least one compound of the formula (2) as described above or described hereinafter are used for a phosphorescent emitter, it is preferable when the triplet energy thereof is not significantly less than the triplet energy of the phosphorescent emitter. In respect of the triplet level, it is preferably the case that T₁(emitter)−T₁(matrix)≤0.2 eV, more preferably ≤0.15 eV, most preferably ≤0.1 eV. T₁(matrix) here is the triplet level of the matrix material in the emission layer, this condition being applicable to each of the two matrix materials, and T₁(emitter) is the triplet level of the phosphorescent emitter. If the emission layer contains more than two matrix materials, the abovementioned relationship is preferably also applicable to every further matrix material.

There follows a description of the host material 1 and its preferred embodiments that is/are present in the device of the invention. The preferred embodiments of the host material 1 of the formula (1) are also applicable to the mixture and/or formulation of the invention.

In compounds of the formula (1), the symbol Y is C(R)₂ or NR.

In a preferred embodiment of the compounds of the formula (1), the symbol Y is preferably C(R)₂.

The invention therefore further provides the electroluminescent device as described above, where Y in the host material 1 is C(R)₂ where R is the same or different at each instance and is selected from a straight-chain alkyl group having 1 to 20 carbon atoms or a branched or cyclic alkyl group having 3 to 20 carbon atoms, an aromatic or heteroaromatic ring system having 5 to 40 ring atoms, or an aralkyl or heteroaralkyl group having 5 to 40 ring atoms, and where two substituents R may form a monocyclic or polycyclic, aliphatic, aromatic or heteroaromatic ring system that may be substituted by one or more R² radicals.

In this embodiment, R is preferably a straight-chain alkyl group having 1 to 4 carbon atoms or phenyl, or the two substituents R together with the carbon to which they are bonded form a cycloalkyl group having 3 to 6 carbon atoms or a spirofluorenyl group, where the cyclic groups mentioned may be substituted by one or more R² radicals. In this embodiment, R is more preferably the same and is a methyl group or phenyl group, or the two substituents R form a cyclopentyl group, a cyclohexyl group or a spirofluorenyl group. In this embodiment, R is most preferably the same and is a methyl group, or the two substituents R form a spirofluorenyl group.

When the two substituents R in the C(R)₂ group form a spirofluorenyl group substituted by R², this can be visualized by the following structure:

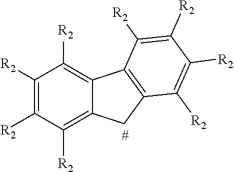

where # marks the carbon atom of the substituent C(R)₂ and R² has a definition given above or hereinafter.

Compounds of the formula (1) in which Y is preferably C(R)₂ can be described by the formula (1a)

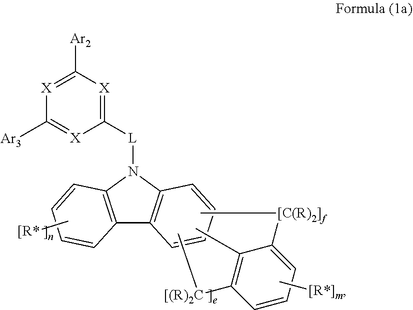

where Ar₂, Ar₃, R*, n, m, L, R and X have a definition given above or a definition given hereinafter or above as preferred.

In a preferred embodiment of the compounds of the formula (1), the symbol Y is preferably NR where R is the same or different at each instance and is selected from a straight-chain alkyl group having 1 to 20 carbon atoms or a branched or cyclic alkyl group having 3 to 20 carbon atoms, an aromatic or heteroaromatic ring system having 5 to 40 ring atoms, or an aralkyl or heteroaralkyl group having 5 to 40 ring atoms.

In this embodiment, R is preferably an aromatic or heteroaromatic ring system having 5 to 40 ring atoms. In this embodiment, R is more preferably phenyl, 1,3-biphenyl or 1,4-biphenyl.

The invention therefore further provides the electroluminescent device as described above, wherein Y in the host material 1 is NR, and R has a definition given above.

Compounds of the formula (1) in which Y is preferably NR can be described by the formula (1b)

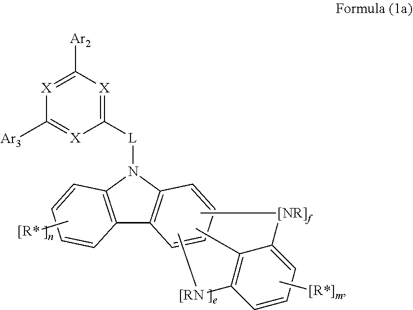

where Ar₂, Ar₃, R*, n, m, L, R and X have a definition given above or a definition given hereinafter or above as preferred.

In compounds of the formulae (1), (1a) and (1b) or preferred embodiments of the host material of the formulae (1), (1a) and (1b), the symbol X is CR⁰ or N, where at least two X groups are N.

The substituent

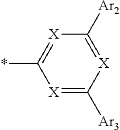

therefore has the following definitions, where * indicates the bonding site via L to the carbazole, and R⁰, Ar₂ and Ar₃ have a definition given above or a definition given as preferred:

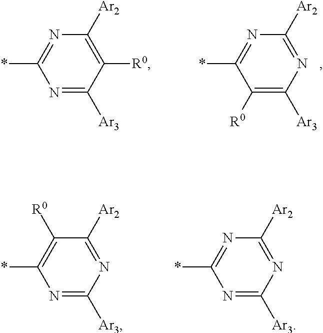

In host material 1, X is preferably N at three instances.

The present invention therefore further provides the electroluminescent device as described above or described as preferred, wherein, in host material 1, the symbol X is N at three instances.

R⁰ is the same or different at each instance and is preferably selected from the group of H, D, CN, a straight-chain or branched alkyl group having 1 to 10 carbon atoms or an aromatic or heteroaromatic ring system that has 5 to 40 ring atoms and may be substituted by one or more R³ radicals. R⁰ at each instance is preferably H, D or an unsubstituted aromatic ring system having 6 to 18 ring atoms. R⁰ at each instance is more preferably H.

In compounds of the formulae (1), (1a) and (1b) or preferred embodiments of the host material of the formulae (1), (1a) and (1b), the linker L is a single bond or a phenylene.

In compounds of the formulae (1), (1a) and (1b) or preferred embodiments of the host material of the formulae (1), (1a) and (1b), the linker L is preferably a bond or a linker selected from the group of L-1, L-2 and L-3,

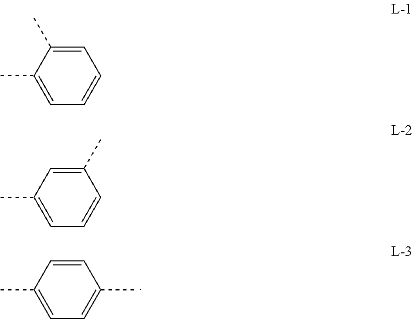

In compounds of the formulae (1), (1a) and (1b) or preferred embodiments of the host material of the formulae (1), (1a) and (1b), the linker L is more preferably a bond or a linker selected from the group of L-2 and L-3.

In compounds of the formulae (1), (1a) and (1b) or preferred embodiments of the host material of the formulae (1), (1a) and (1b), the linker L is most preferably a bond.

In compounds of the formulae (1), (1a) and (1b) or preferred embodiments of the host material of the formulae (1), (1a) and (1b), n is preferably 0, 1 or 2, more preferably 0, where R* has a preferred definition given above or given hereinafter.

In compounds of the formulae (1), (1a) and (1b) or preferred embodiments of the host material of the formulae (1), (1a) and (1b), m is preferably 0, 1 or 2, more preferably 0, where R* has a preferred definition given above or given hereinafter.

R* is the same or different at each instance and is preferably selected from the group of D or an aromatic or heteroaromatic ring system which has 6 to 18 ring atoms and may be partly or fully deuterated. R* at each instance is preferably phenyl, 1,3-biphenyl, 1,4-biphenyl, dibenzofuranyl or dibenzothiophenyl. R* at each instance is more preferably phenyl, 1,3-biphenyl, 1,4-biphenyl or dibenzofuranyl.

Compounds of the formula (1a) are preferred embodiments of the compounds of the formula (1) and of the host material 1.

In compounds of the formulae (1), (1a) and (1b) or preferred embodiments of the host material of the formulae (1), (1a) and (1b), Ar₂ at each instance is preferably a biphenyl, a dibenzofuranyl, a dibenzothiophenyl, a carbazol-N-yl or a carbazol-N-yl-phenyl group that may be substituted by one or more preferred R* radicals.

In compounds of the formulae (1), (1a) and (1b) or preferred embodiments of the host material of the formulae (1), (1a) and (1b), Ar₂ at each instance is more preferably a dibenzofuranyl, a dibenzothiophenyl or a carbazol-N-yl group that is unsubstituted or monosubstituted by phenyl.

In compounds of the formulae (1), (1a) and (1b) or preferred embodiments of the host material of the formulae (1), (1a) and (1b), Ar₂ at each instance is more preferably a biphenyl group that is preferably unsubstituted.

In compounds of the formulae (1), (1a) and (1b) or preferred embodiments of the host material of the formulae (1), (1a) and (1b), Ar₂ at each instance is more preferably a carbazol-N-yl-phenyl group that is preferably unsubstituted.

What is meant by “Ar₂ and Ar₃ are always different” is that either the position of the linkage to the radical of the formulae (1), (1a) and (1b) is different or the structures of Ar₂ and Ar₃ are different. Different positions of the linkage of two dibenzofuranyl groups, for example, also have the effect that the compound of the formulae (1), (1a) and (1b) is unsymmetrically substituted. The structures of Ar₂ and Ar₃ are preferably different from the structure.

In compounds of the formulae (1), (1a) and (1b) or preferred embodiments of the host material of the formulae (1), (1a) and (1b), Ar₂ and Ar₃ are always different, and Ar₃ may preferably be selected from the following groups Ar-1 to Ar-19, where R², R³ and Ar₁ have a definition given above or given with preference, and where R², R³ or Ar₁ cannot bond two heteroatoms directly to one another:

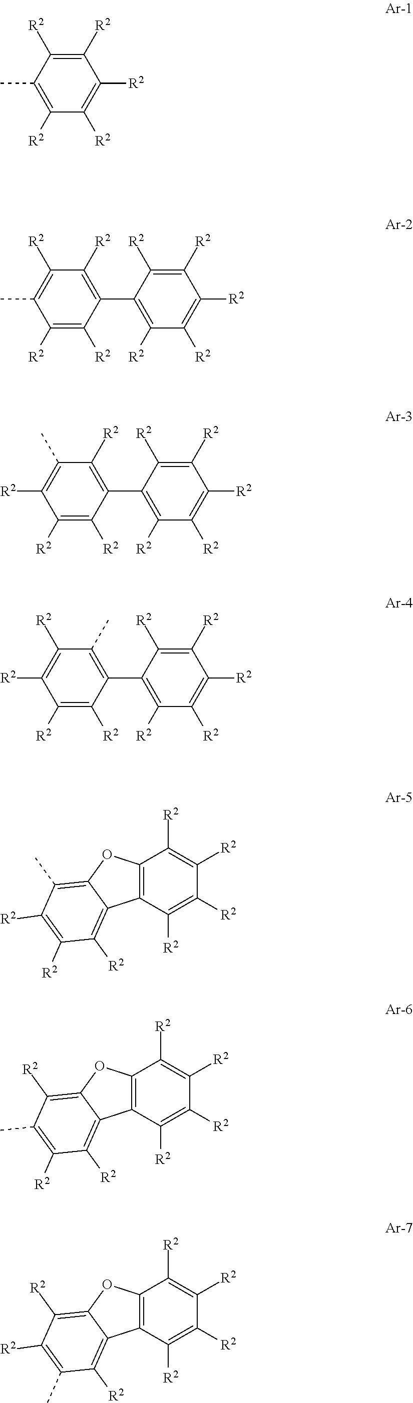

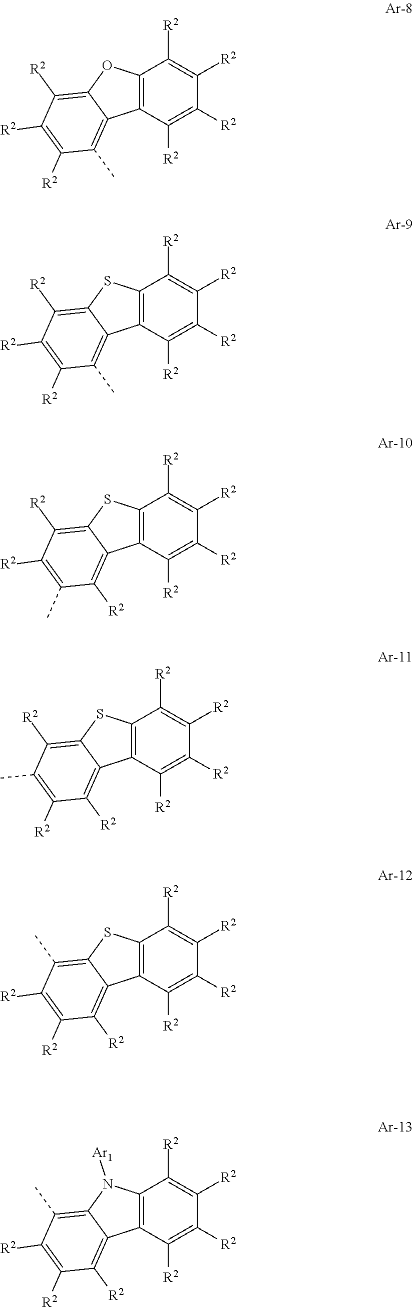

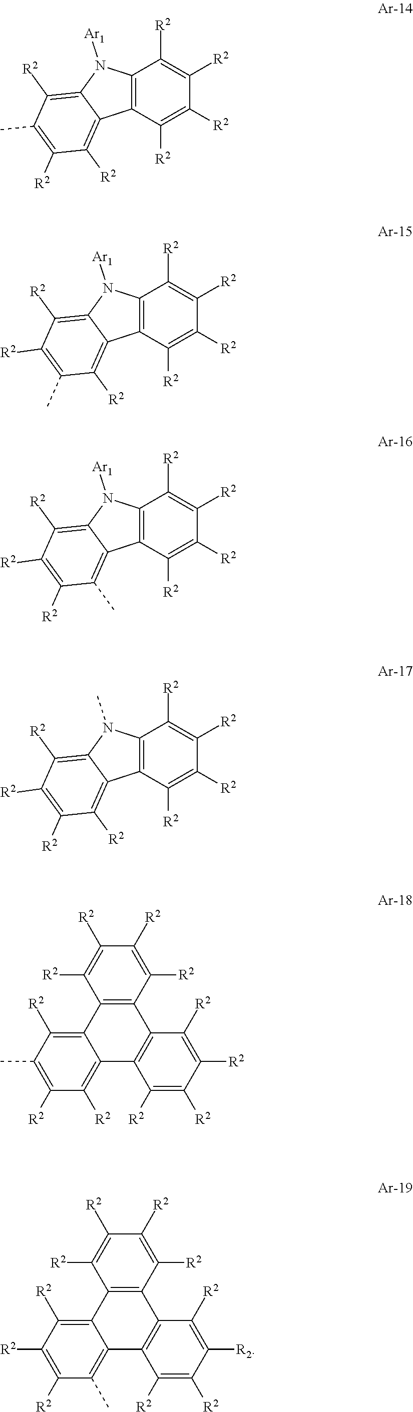

The dotted line indicates the bonding site to the radical of the formulae (1), (1a) or (1b).

More preferably, Ar₃ is Ar-1 to Ar-12 and Ar-17, where R² and Ar₁ have a definition specified above or specified as preferred hereinafter.

R² in substituents of the formulae Ar-1 to Ar-19, as described above, is preferably selected from the group of H, D, CN, an aromatic or heteroaromatic ring system which has 5 to 40 ring atoms and may be substituted in each case by one or more R³ radicals.

R² in substituents of the formulae Ar-1 to Ar-19, as described above, is more preferably D, phenyl or N-carbazolyl.

Ar₁ in substituents of the formulae Ar-13 to Ar-16, as described above, is preferably phenyl.

R³ in compounds of the formulae (1), (1a) and (1b), as described above or described as preferred, is preferably selected independently at each instance from the group of H, CN, an aromatic or heteroaromatic ring system having 5 to 40 ring atoms in which one or more hydrogen atoms may be replaced by D or CN. R³ in compounds of the formulae (1), (1a) and (1b), as described above or described as preferred, is more preferably selected independently at each instance from H, phenyl or deuterated phenyl.

In compounds of the formulae (1), (1a) and (1b) or preferred embodiments of the host material of the formulae (1), (1a) and (1b), Ar₂ and Ar₃ are always different and Ar₃ may more preferably be selected from Ar-1 and Ar-2, where R² has a definition given above or given as preferred.

The linkage of the ring fused on via Y is not limited in any way and may be via any possible position. Preferred compounds of the formula (1) are accordingly compounds of the formulae (1c) to (1h):

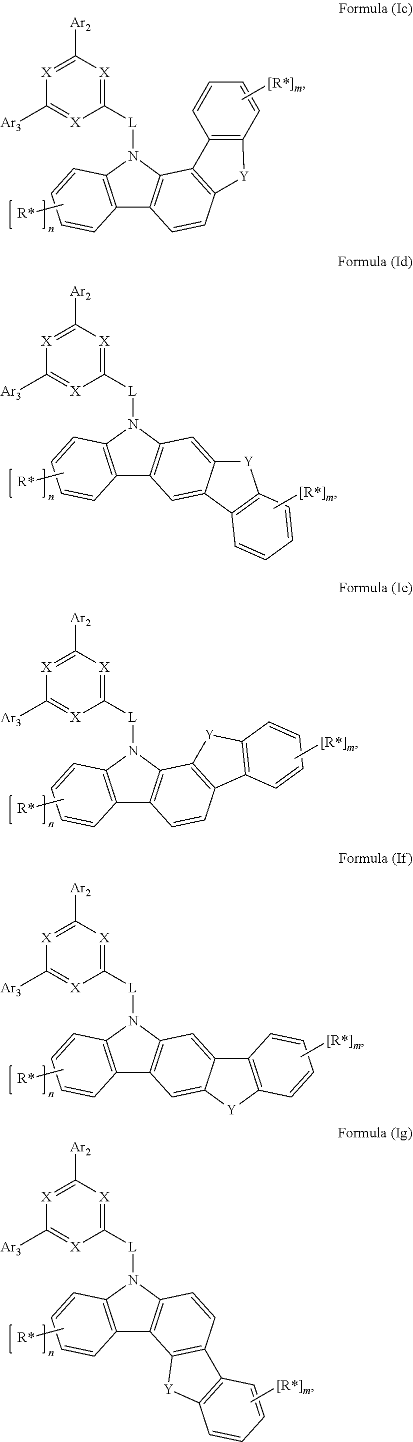

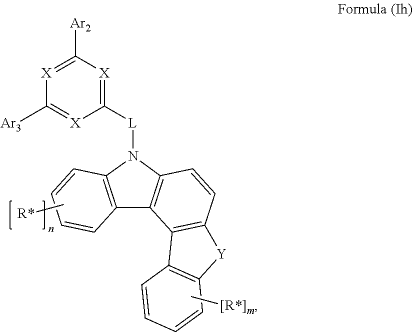

where Ar₂, Ar₃, R*, n, m, L, X and Y have a definition given above or given above as preferred.

Examples of suitable host materials of the formulae (1), (1a), (1b), (1c), (1d), (1e), (1f), (1g) and (1h) that are selected in accordance with the invention and are preferably used in combination with at least one compound of the formula (2) in the electroluminescent device of the invention are the structures given below in table 1.

TABLE 1

| | |
|---|---|
| 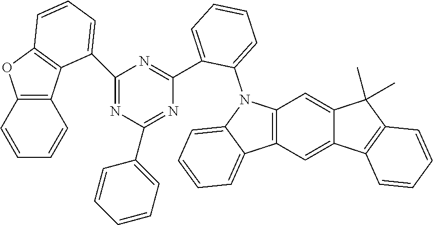 [SMILES: CC1(C)c2ccccc2-c2cc3c4ccccc4n(-c4ccccc4-c4nc(-c5ccccc5)nc(-c5cccc6oc7ccccc7c56)n4)c3cc21] | 1 |
| 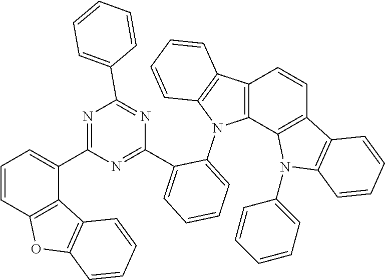 [SMILES: c1ccc(-c2nc(-c3ccccc3-n3c4ccccc4c4ccc5c6ccccc6n(-c6ccccc6)c5c43)nc(-c3cccc4oc5ccccc5c34)n2)cc1] | 2 |
| 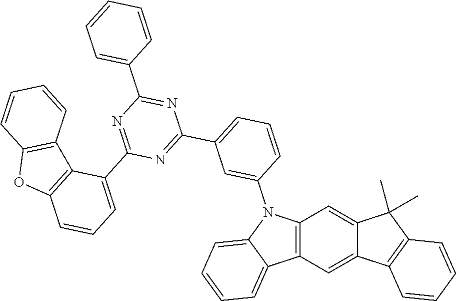 [SMILES: CC1(C)c2ccccc2-c2cc3c4ccccc4n(-c4cccc(-c5nc(-c6ccccc6)nc(-c6cccc7oc8ccccc8c67)n5)c4)c3cc21] | 3 |
| 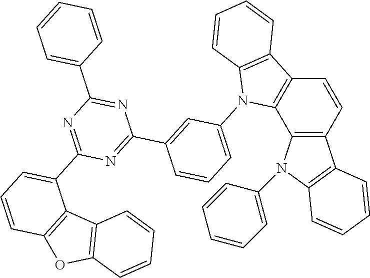 [SMILES: c1ccc(-c2nc(-c3cccc(-n4c5ccccc5c5ccc6c7ccccc7n(-c7ccccc7)c6c54)c3)nc(-c3cccc4oc5ccccc5c34)n2)cc1] | 4 |
| 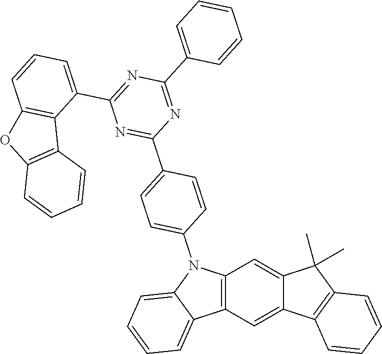 [SMILES: CC1(C)c2ccccc2-c2cc3c4ccccc4n(-c4ccc(-c5nc(-c6ccccc6)nc(-c6cccc7oc8ccccc8c67)n5)cc4)c3cc21] | 5 |
| 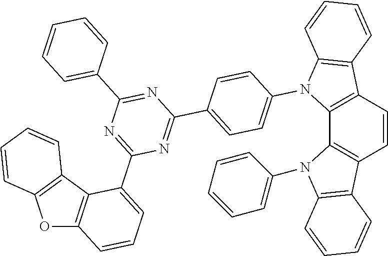 [SMILES: c1ccc(-c2nc(-c3ccc(-n4c5ccccc5c5ccc6c7ccccc7n(-c7ccccc7)c6c54)cc3)nc(-c3cccc4oc5ccccc5c34)n2)cc1] | 6 |
| 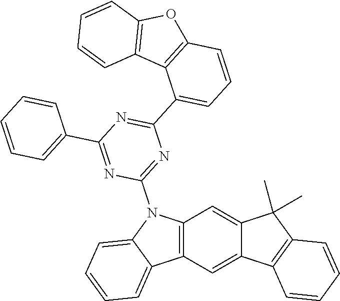 [SMILES: CC1(C)c2ccccc2-c2cc3c4ccccc4n(-c4nc(-c5ccccc5)nc(-c5cccc6oc7ccccc7c56)n4)c3cc21] | 7 |
| 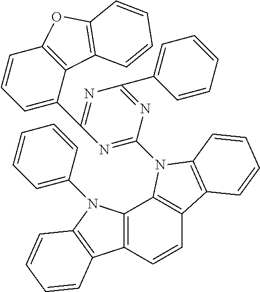 [SMILES: c1ccc(-c2nc(-c3cccc4oc5ccccc5c34)nc(-n3c4ccccc4c4ccc5c6ccccc6n(-c6ccccc6)c5c43)n2)cc1] | 8 |
| 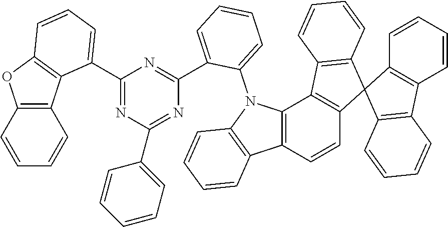 [SMILES: c1ccc(-c2nc(-c3ccccc3-n3c4ccccc4c4ccc5c(c43)-c3ccccc3C53c4ccccc4-c4ccccc43)nc(-c3cccc4oc5ccccc5c34)n2)cc1] | 9 |
| 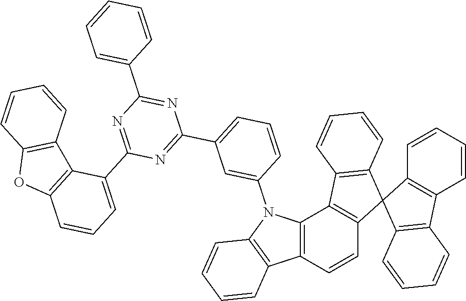 [SMILES: c1ccc(-c2nc(-c3cccc(-n4c5ccccc5c5ccc6c(c54)-c4ccccc4C64c5ccccc5-c5ccccc54)c3)nc(-c3cccc4oc5ccccc5c34)n2)cc1] | 10 |
| 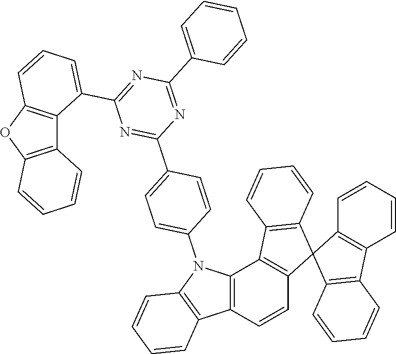 [SMILES: c1ccc(-c2nc(-c3ccc(-n4c5ccccc5c5ccc6c(c54)-c4ccccc4C64c5ccccc5-c5ccccc54)cc3)nc(-c3cccc4oc5ccccc5c34)n2)cc1] | 11 |
| 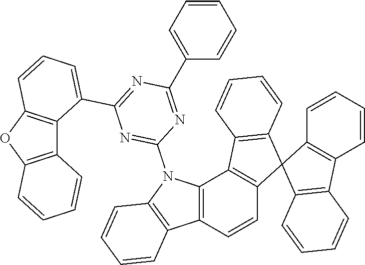 [SMILES: c1ccc(-c2nc(-c3cccc4oc5ccccc5c34)nc(-n3c4ccccc4c4ccc5c(c43)-c3ccccc3C53c4ccccc4-c4ccccc43)n2)cc1] | 12 |
| 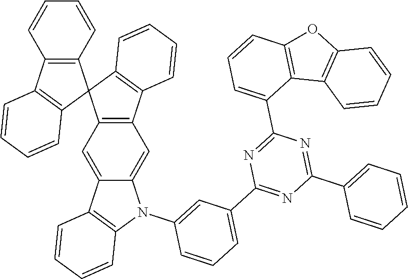 [SMILES: c1ccc(-c2nc(-c3cccc(-n4c5ccccc5c5cc6c(cc54)-c4ccccc4C64c5ccccc5-c5ccccc54)c3)nc(-c3cccc4oc5ccccc5c34)n2)cc1] | 13 |
| 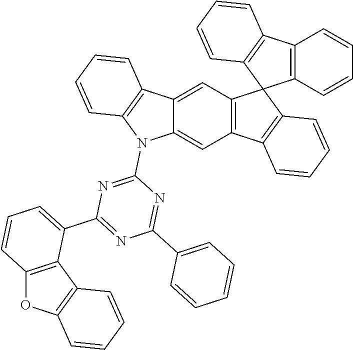 [SMILES: c1ccc(-c2nc(-c3cccc4oc5ccccc5c34)nc(-n3c4ccccc4c4cc5c(cc43)-c3ccccc3C53c4ccccc4-c4ccccc43)n2)cc1] | 14 |
| 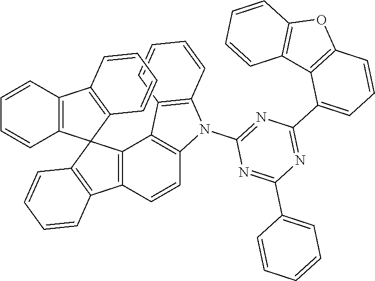 [SMILES: c1ccc(-c2nc(-c3cccc4oc5ccccc5c34)nc(-n3c4ccccc4c4c5c(ccc43)-c3ccccc3C53c4ccccc4-c4ccccc43)n2)cc1] | 15 |
| 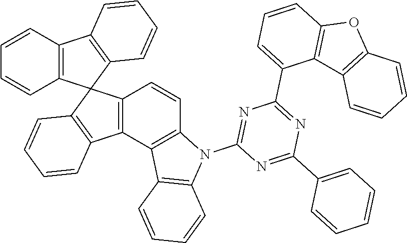 [SMILES: c1ccc(-c2nc(-c3cccc4oc5ccccc5c34)nc(-n3c4ccccc4c4c5c(ccc43)C3(c4ccccc4-c4ccccc43)c3ccccc3-5)n2)cc1] | 16 |
| 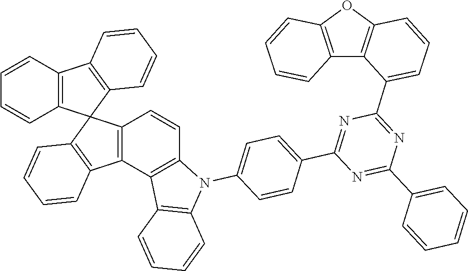 [SMILES: c1ccc(-c2nc(-c3ccc(-n4c5ccccc5c5c6c(ccc54)C4(c5ccccc5-c5ccccc54)c4ccccc4-6)cc3)nc(-c3cccc4oc5ccccc5c34)n2)cc1] | 17 |
| 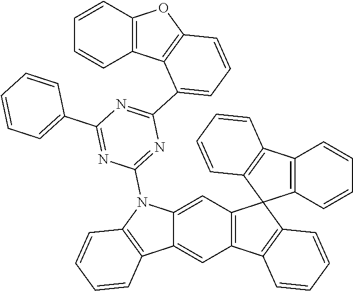 [SMILES: c1ccc(-c2nc(-c3cccc4oc5ccccc5c34)nc(-n3c4ccccc4c4cc5c(cc43)C3(c4ccccc4-c4ccccc43)c3ccccc3-5)n2)cc1] | 18 |
| 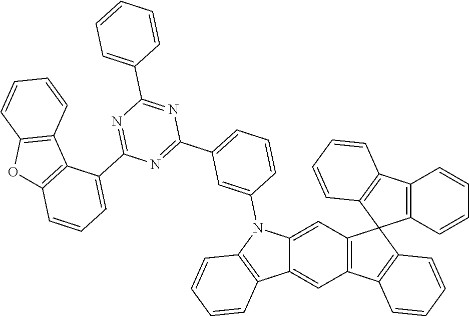 [SMILES: c1ccc(-c2nc(-c3cccc(-n4c5ccccc5c5cc6c(cc54)C4(c5ccccc5-c5ccccc54)c4ccccc4-6)c3)nc(-c3cccc4oc5ccccc5c34)n2)cc1] | 19 |
| 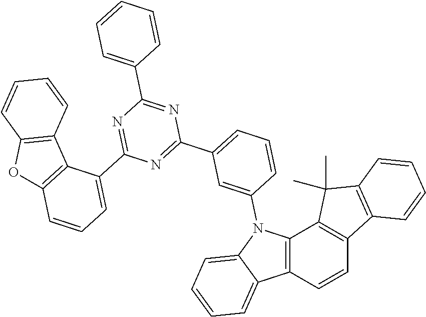 [SMILES: CC1(C)c2ccccc2-c2ccc3c4ccccc4n(-c4cccc(-c5nc(-c6ccccc6)nc(-c6cccc7oc8ccccc8c67)n5)c4)c3c21] | 20 |
| 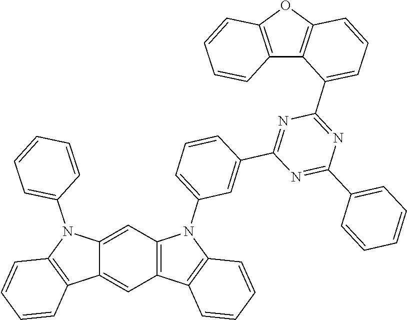 [SMILES: c1ccc(-c2nc(-c3cccc(-n4c5ccccc5c5cc6c7ccccc7n(-c7ccccc7)c6cc54)c3)nc(-c3cccc4oc5ccccc5c34)n2)cc1] | 21 |
| 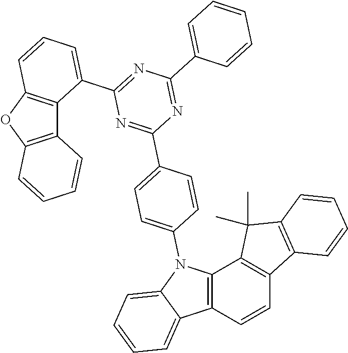 [SMILES: CC1(C)c2ccccc2-c2ccc3c4ccccc4n(-c4ccc(-c5nc(-c6ccccc6)nc(-c6cccc7oc8ccccc8c67)n5)cc4)c3c21] | 22 |
| 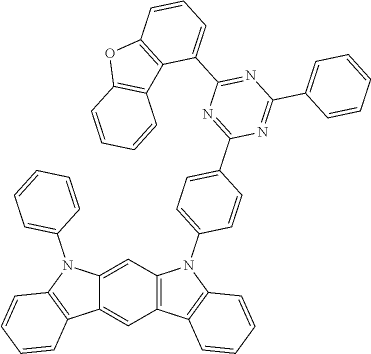 [SMILES: c1ccc(-c2nc(-c3ccc(-n4c5ccccc5c5cc6c7ccccc7n(-c7ccccc7)c6cc54)cc3)nc(-c3cccc4oc5ccccc5c34)n2)cc1] | 23 |
| 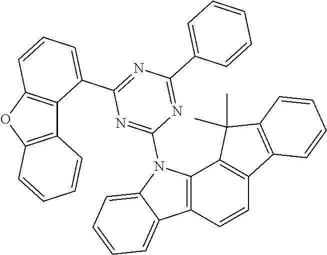 [SMILES: CC1(C)c2ccccc2-c2ccc3c4ccccc4n(-c4nc(-c5ccccc5)nc(-c5cccc6oc7ccccc7c56)n4)c3c21] | 24 |
| 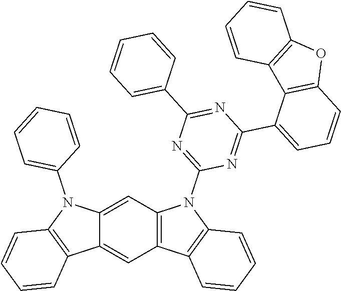 [SMILES: c1ccc(-c2nc(-c3cccc4oc5ccccc5c34)nc(-n3c4ccccc4c4cc5c6ccccc6n(-c6ccccc6)c5cc43)n2)cc1] | 25 |
| 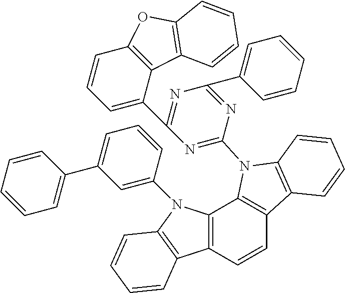 [SMILES: c1ccc(-c2cccc(-n3c4ccccc4c4ccc5c6ccccc6n(-c6nc(-c7ccccc7)nc(-c7cccc8oc9ccccc9c78)n6)c5c43)c2)cc1] | 26 |
| 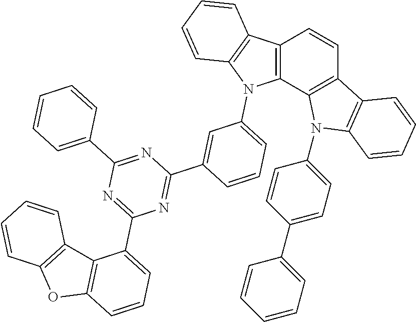 [SMILES: c1ccc(-c2ccc(-n3c4ccccc4c4ccc5c6ccccc6n(-c6cccc(-c7nc(-c8ccccc8)nc(-c8cccc9oc%10ccccc%10c89)n7)c6)c5c43)cc2)cc1] | 27 |
| 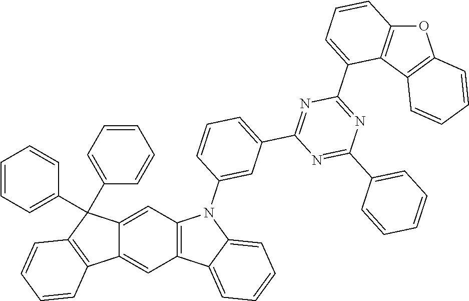 [SMILES: c1ccc(-c2nc(-c3cccc(-n4c5ccccc5c5cc6c(cc54)C(c4ccccc4)(c4ccccc4)c4ccccc4-6)c3)nc(-c3cccc4oc5ccccc5c34)n2)cc1] | 28 |
| 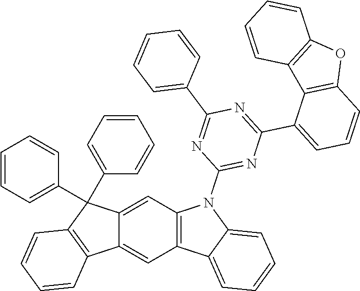 [SMILES: c1ccc(-c2nc(-c3cccc4oc5ccccc5c34)nc(-n3c4ccccc4c4cc5c(cc43)C(c3ccccc3)(c3ccccc3)c3ccccc3-5)n2)cc1] | 29 |
| 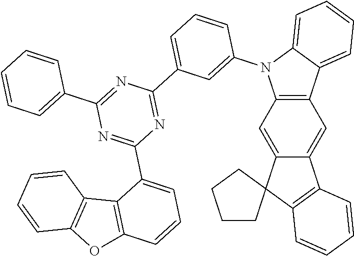 [SMILES: c1ccc(-c2nc(-c3cccc(-n4c5ccccc5c5cc6c(cc54)C4(CCCC4)c4ccccc4-6)c3)nc(-c3cccc4oc5ccccc5c34)n2)cc1] | 30 |
| 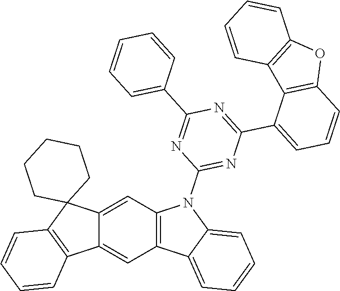 [SMILES: c1ccc(-c2nc(-c3cccc4oc5ccccc5c34)nc(-n3c4ccccc4c4cc5c(cc43)C3(CCCCC3)c3ccccc3-5)n2)cc1] | 31 |
| 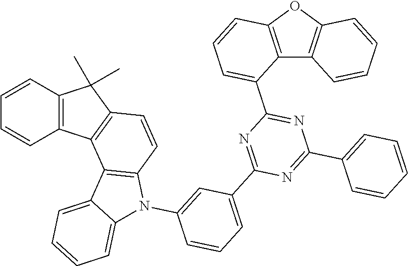 [SMILES: CC1(C)c2ccccc2-c2c1ccc1c2c2ccccc2n1-c1cccc(-c2nc(-c3ccccc3)nc(-c3cccc4oc5ccccc5c34)n2)c1] | 32 |
| 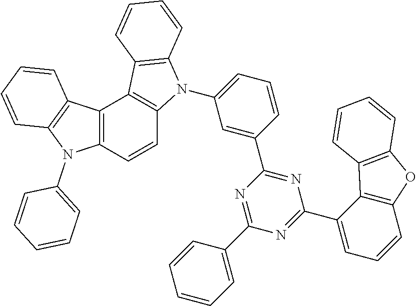 [SMILES: c1ccc(-c2nc(-c3cccc(-n4c5ccccc5c5c6c7ccccc7n(-c7ccccc7)c6ccc54)c3)nc(-c3cccc4oc5ccccc5c34)n2)cc1] | 33 |
| 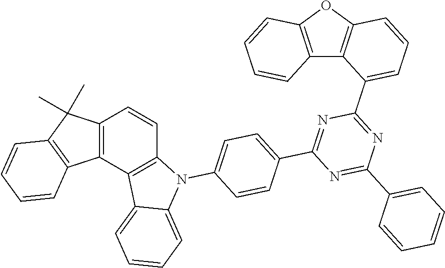 [SMILES: CC1(C)c2ccccc2-c2c1ccc1c2c2ccccc2n1-c1ccc(-c2nc(-c3ccccc3)nc(-c3cccc4oc5ccccc5c34)n2)cc1] | 34 |
| 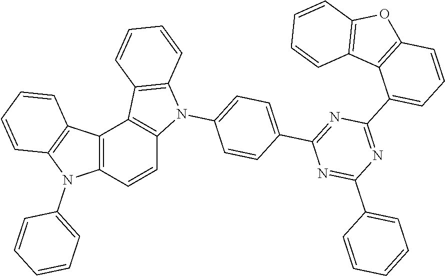 [SMILES: c1ccc(-c2nc(-c3ccc(-n4c5ccccc5c5c6c7ccccc7n(-c7ccccc7)c6ccc54)cc3)nc(-c3cccc4oc5ccccc5c34)n2)cc1] | 35 |
| 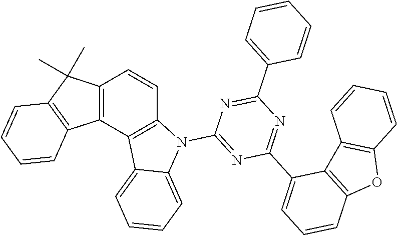 [SMILES: CC1(C)c2ccccc2-c2c1ccc1c2c2ccccc2n1-c1nc(-c2ccccc2)nc(-c2cccc3oc4ccccc4c23)n1] | 36 |
| 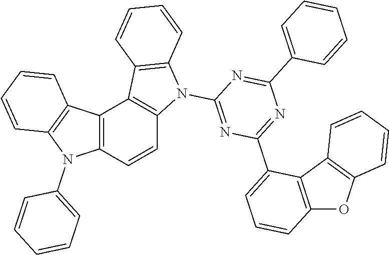 [SMILES: c1ccc(-c2nc(-c3cccc4oc5ccccc5c34)nc(-n3c4ccccc4c4c5c6ccccc6n(-c6ccccc6)c5ccc43)n2)cc1] | 37 |
| 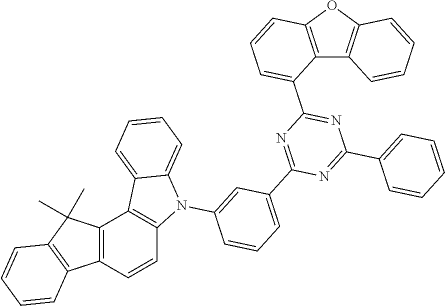 [SMILES: CC1(C)c2ccccc2-c2ccc3c(c21)c1ccccc1n3-c1cccc(-c2nc(-c3ccccc3)nc(-c3cccc4oc5ccccc5c34)n2)c1] | 38 |
| 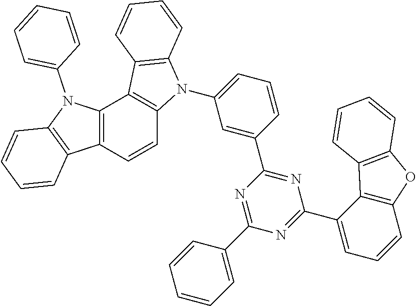 [SMILES: c1ccc(-c2nc(-c3cccc(-n4c5ccccc5c5c4ccc4c6ccccc6n(-c6ccccc6)c45)c3)nc(-c3cccc4oc5ccccc5c34)n2)cc1] | 39 |
| 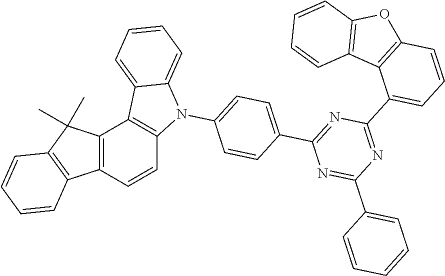 [SMILES: CC1(C)c2ccccc2-c2ccc3c(c21)c1ccccc1n3-c1ccc(-c2nc(-c3ccccc3)nc(-c3cccc4oc5ccccc5c34)n2)cc1] | 40 |
| 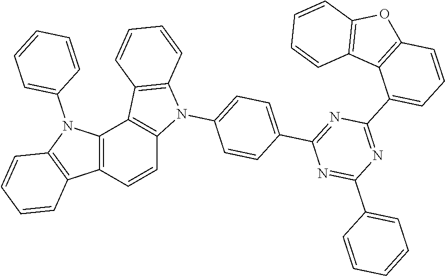 [SMILES: c1ccc(-c2nc(-c3ccc(-n4c5ccccc5c5c4ccc4c6ccccc6n(-c6ccccc6)c45)cc3)nc(-c3cccc4oc5ccccc5c34)n2)cc1] | 41 |
| 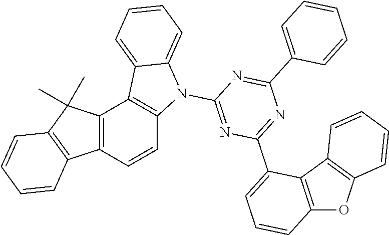 [SMILES: CC1(C)c2ccccc2-c2ccc3c(c21)c1ccccc1n3-c1nc(-c2ccccc2)nc(-c2cccc3oc4ccccc4c23)n1] | 42 |
| 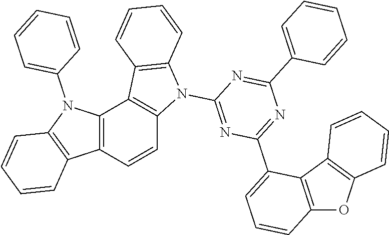 [SMILES: c1ccc(-c2nc(-c3cccc4oc5ccccc5c34)nc(-n3c4ccccc4c4c3ccc3c5ccccc5n(-c5ccccc5)c34)n2)cc1] | 43 |
| 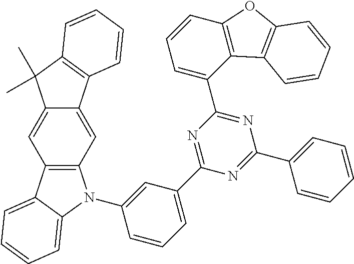 [SMILES: CC1(C)c2ccccc2-c2cc3c(cc21)c1ccccc1n3-c1cccc(-c2nc(-c3ccccc3)nc(-c3cccc4oc5ccccc5c34)n2)c1] | 44 |
| 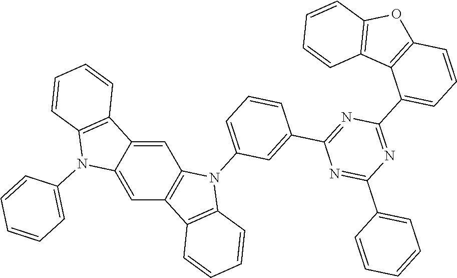 [SMILES: c1ccc(-c2nc(-c3cccc(-n4c5ccccc5c5cc6c(cc54)c4ccccc4n6-c4ccccc4)c3)nc(-c3cccc4oc5ccccc5c34)n2)cc1] | 45 |
| 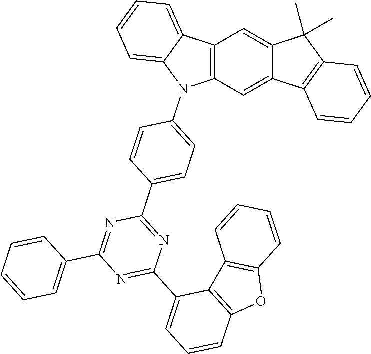 [SMILES: CC1(C)c2ccccc2-c2cc3c(cc21)c1ccccc1n3-c1ccc(-c2nc(-c3ccccc3)nc(-c3cccc4oc5ccccc5c34)n2)cc1] | 46 |
| 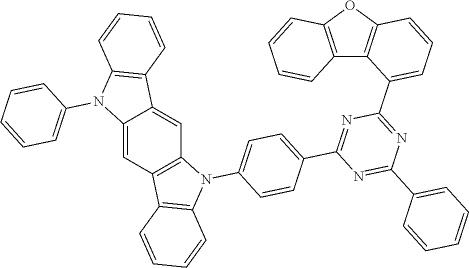 [SMILES: c1ccc(-c2nc(-c3ccc(-n4c5ccccc5c5cc6c(cc54)c4ccccc4n6-c4ccccc4)cc3)nc(-c3cccc4oc5ccccc5c34)n2)cc1] | 47 |
| 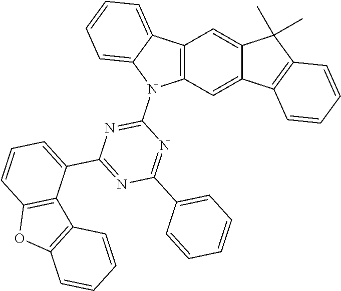 [SMILES: CC1(C)c2ccccc2-c2cc3c(cc21)c1ccccc1n3-c1nc(-c2ccccc2)nc(-c2cccc3oc4ccccc4c23)n1] | 48 |
| 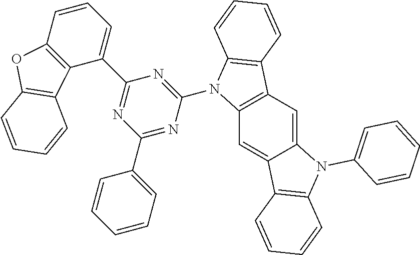 [SMILES: c1ccc(-c2nc(-c3cccc4oc5ccccc5c34)nc(-n3c4ccccc4c4cc5c(cc43)c3ccccc3n5-c3ccccc3)n2)cc1] | 49 |
| 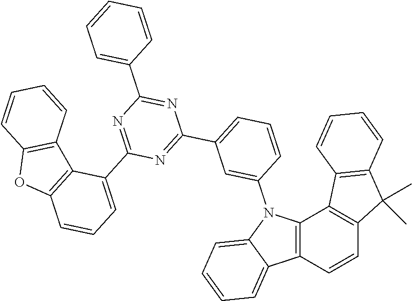 [SMILES: CC1(C)c2ccccc2-c2c1ccc1c3ccccc3n(-c3cccc(-c4nc(-c5ccccc5)nc(-c5cccc6oc7ccccc7c56)n4)c3)c21] | 50 |
| 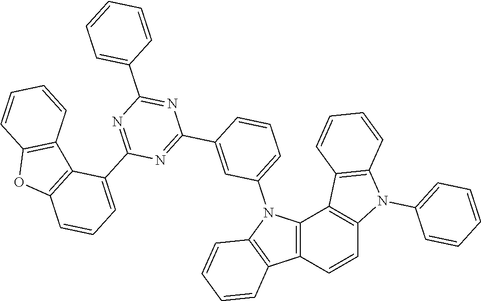 [SMILES: c1ccc(-c2nc(-c3cccc(-n4c5ccccc5c5ccc6c(c7ccccc7n6-c6ccccc6)c54)c3)nc(-c3cccc4oc5ccccc5c34)n2)cc1] | 51 |
| 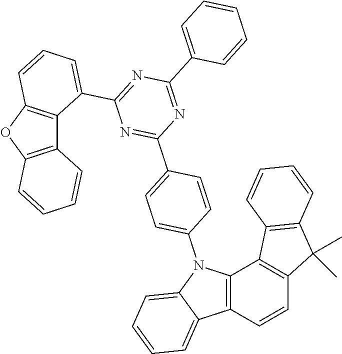 [SMILES: CC1(C)c2ccccc2-c2c1ccc1c3ccccc3n(-c3ccc(-c4nc(-c5ccccc5)nc(-c5cccc6oc7ccccc7c56)n4)cc3)c21] | 52 |
| 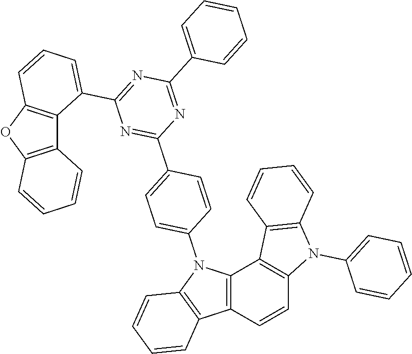 [SMILES: c1ccc(-c2nc(-c3ccc(-n4c5ccccc5c5ccc6c(c7ccccc7n6-c6ccccc6)c54)cc3)nc(-c3cccc4oc5ccccc5c34)n2)cc1] | 53 |
| 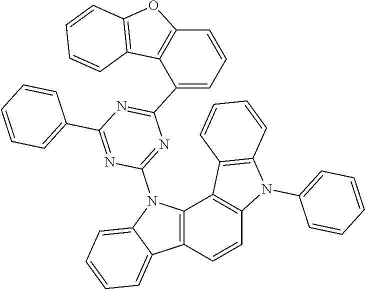 [SMILES: c1ccc(-c2nc(-c3cccc4oc5ccccc5c34)nc(-n3c4ccccc4c4ccc5c(c6ccccc6n5-c5ccccc5)c43)n2)cc1] | 54 |
| 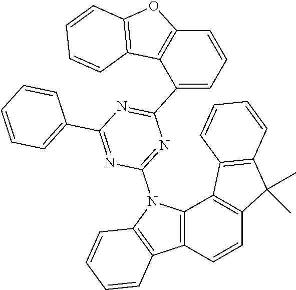 [SMILES: CC1(C)c2ccccc2-c2c1ccc1c3ccccc3n(-c3nc(-c4ccccc4)nc(-c4cccc5oc6ccccc6c45)n3)c21] | 55 |
| 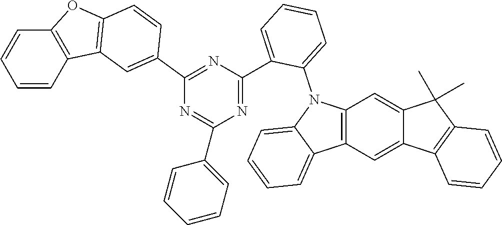 [SMILES: CC1(C)c2ccccc2-c2cc3c4ccccc4n(-c4ccccc4-c4nc(-c5ccccc5)nc(-c5ccc6oc7ccccc7c6c5)n4)c3cc21] | 56 |
| 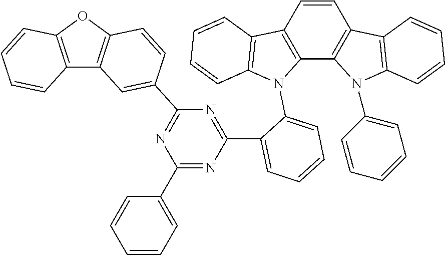 [SMILES: c1ccc(-c2nc(-c3ccc4oc5ccccc5c4c3)nc(-c3ccccc3-n3c4ccccc4c4ccc5c6ccccc6n(-c6ccccc6)c5c43)n2)cc1] | 57 |
| 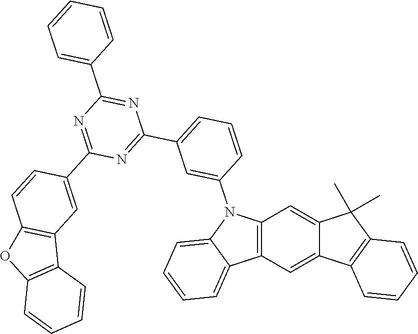 [SMILES: CC1(C)c2ccccc2-c2cc3c4ccccc4n(-c4cccc(-c5nc(-c6ccccc6)nc(-c6ccc7oc8ccccc8c7c6)n5)c4)c3cc21] | 58 |
| 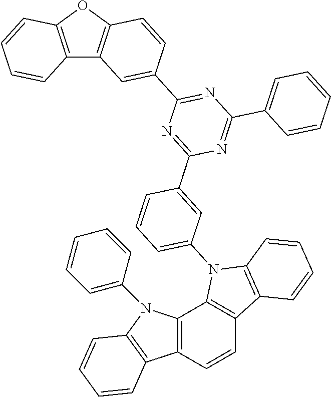 [SMILES: c1ccc(-c2nc(-c3cccc(-n4c5ccccc5c5ccc6c7ccccc7n(-c7ccccc7)c6c54)c3)nc(-c3ccc4oc5ccccc5c4c3)n2)cc1] | 59 |
| 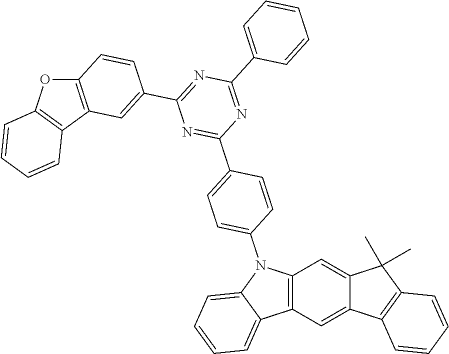 [SMILES: CC1(C)c2ccccc2-c2cc3c4ccccc4n(-c4ccc(-c5nc(-c6ccccc6)nc(-c6ccc7oc8ccccc8c7c6)n5)cc4)c3cc21] | 60 |
| 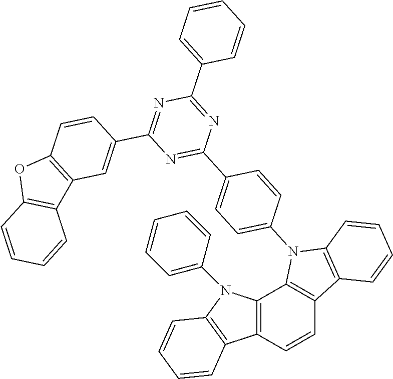 [SMILES: c1ccc(-c2nc(-c3ccc(-n4c5ccccc5c5ccc6c7ccccc7n(-c7ccccc7)c6c54)cc3)nc(-c3ccc4oc5ccccc5c4c3)n2)cc1] | 61 |
| 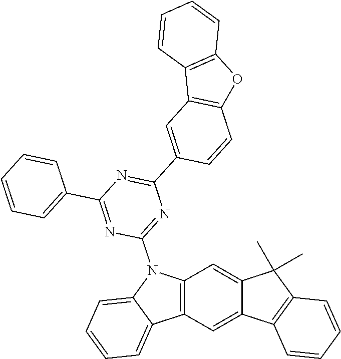 [SMILES: CC1(C)c2ccccc2-c2cc3c4ccccc4n(-c4nc(-c5ccccc5)nc(-c5ccc6oc7ccccc7c6c5)n4)c3cc21] | 62 |
| 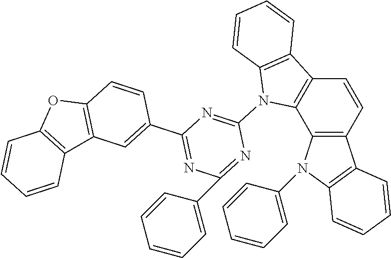 [SMILES: c1ccc(-c2nc(-c3ccc4oc5ccccc5c4c3)nc(-n3c4ccccc4c4ccc5c6ccccc6n(-c6ccccc6)c5c43)n2)cc1] | 63 |
| 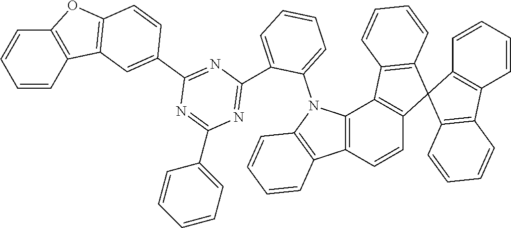 [SMILES: c1ccc(-c2nc(-c3ccc4oc5ccccc5c4c3)nc(-c3ccccc3-n3c4ccccc4c4ccc5c(c43)-c3ccccc3C53c4ccccc4-c4ccccc43)n2)cc1] | 64 |
| 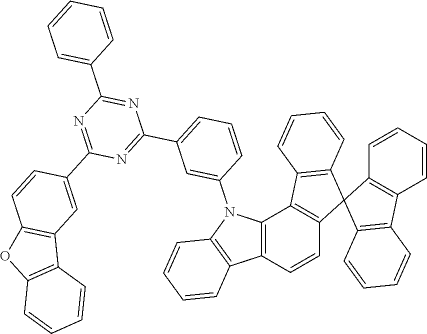 [SMILES: c1ccc(-c2nc(-c3cccc(-n4c5ccccc5c5ccc6c(c54)-c4ccccc4C64c5ccccc5-c5ccccc54)c3)nc(-c3ccc4oc5ccccc5c4c3)n2)cc1] | 65 |
| 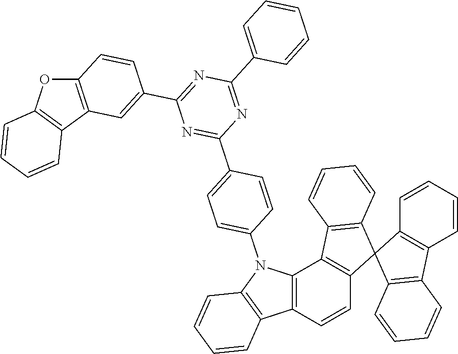 [SMILES: c1ccc(-c2nc(-c3ccc(-n4c5ccccc5c5ccc6c(c54)-c4ccccc4C64c5ccccc5-c5ccccc54)cc3)nc(-c3ccc4oc5ccccc5c4c3)n2)cc1] | 66 |
| 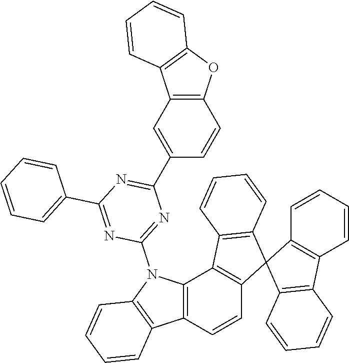 [SMILES: c1ccc(-c2nc(-c3ccc4oc5ccccc5c4c3)nc(-n3c4ccccc4c4ccc5c(c43)-c3ccccc3C53c4ccccc4-c4ccccc43)n2)cc1] | 67 |
| 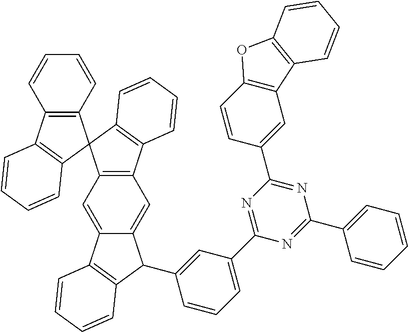 [SMILES: c1ccc(-c2nc(-c3cccc(C4c5ccccc5-c5cc6c(cc54)-c4ccccc4C64c5ccccc5-c5ccccc54)c3)nc(-c3ccc4oc5ccccc5c4c3)n2)cc1] | 68 |
| 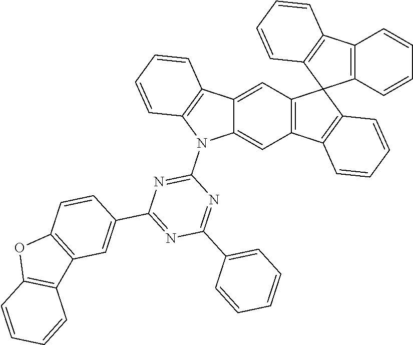 [SMILES: c1ccc(-c2nc(-c3ccc4oc5ccccc5c4c3)nc(-n3c4ccccc4c4cc5c(cc43)-c3ccccc3C53c4ccccc4-c4ccccc43)n2)cc1] | 69 |
| 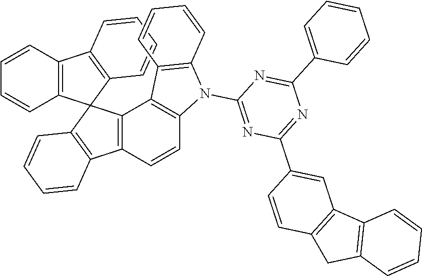 [SMILES: c1ccc(-c2nc(-c3ccc4c(c3)-c3ccccc3C4)nc(-n3c4ccccc4c4c5c(ccc43)-c3ccccc3C53c4ccccc4-c4ccccc43)n2)cc1] | 70 |
| 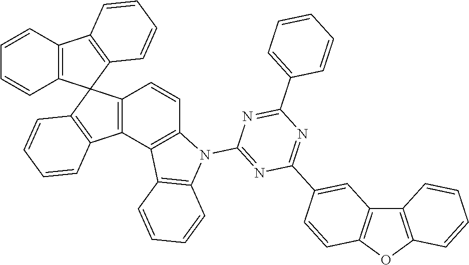 [SMILES: c1ccc(-c2nc(-c3ccc4oc5ccccc5c4c3)nc(-n3c4ccccc4c4c5c(ccc43)C3(c4ccccc4-c4ccccc43)c3ccccc3-5)n2)cc1] | 71 |
| 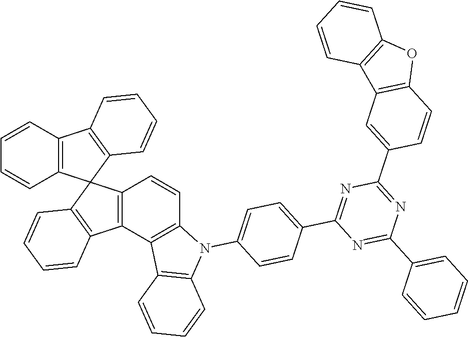 [SMILES: c1ccc(-c2nc(-c3ccc(-n4c5ccccc5c5c6c(ccc54)C4(c5ccccc5-c5ccccc54)c4ccccc4-6)cc3)nc(-c3ccc4oc5ccccc5c4c3)n2)cc1] | 72 |
| 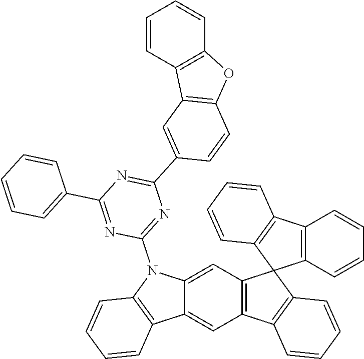 [SMILES: c1ccc(-c2nc(-c3ccc4oc5ccccc5c4c3)nc(-n3c4ccccc4c4cc5c(cc43)C3(c4ccccc4-c4ccccc43)c3ccccc3-5)n2)cc1] | 73 |
| 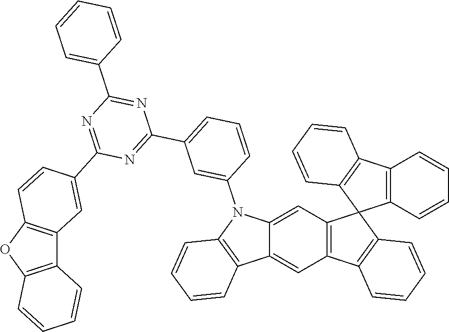 [SMILES: c1ccc(-c2nc(-c3cccc(-n4c5ccccc5c5cc6c(cc54)C4(c5ccccc5-c5ccccc54)c4ccccc4-6)c3)nc(-c3ccc4oc5ccccc5c4c3)n2)cc1] | 74 |
| 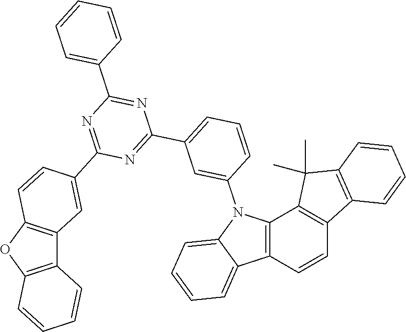 [SMILES: CC1(C)c2ccccc2-c2ccc3c4ccccc4n(-c4cccc(-c5nc(-c6ccccc6)nc(-c6ccc7oc8ccccc8c7c6)n5)c4)c3c21] | 75 |
| 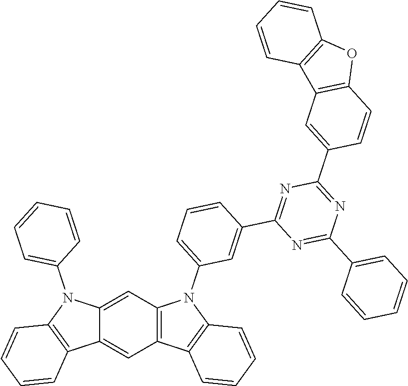 [SMILES: c1ccc(-c2nc(-c3cccc(-n4c5ccccc5c5cc6c7ccccc7n(-c7ccccc7)c6cc54)c3)nc(-c3ccc4oc5ccccc5c4c3)n2)cc1] | 76 |
| 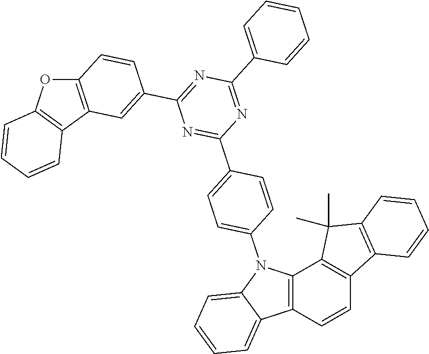 [SMILES: CC1(C)c2ccccc2-c2ccc3c4ccccc4n(-c4ccc(-c5nc(-c6ccccc6)nc(-c6ccc7oc8ccccc8c7c6)n5)cc4)c3c21] | 77 |
| 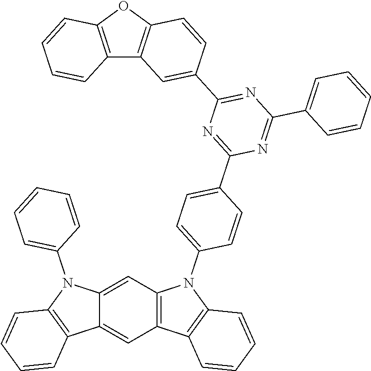 [SMILES: c1ccc(-c2nc(-c3ccc(-n4c5ccccc5c5cc6c7ccccc7n(-c7ccccc7)c6cc54)cc3)nc(-c3ccc4oc5ccccc5c4c3)n2)cc1] | 78 |
| 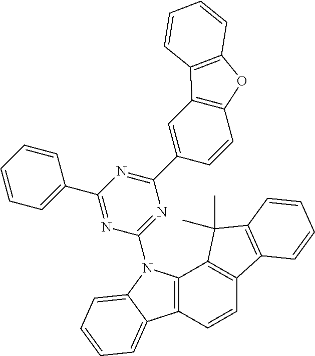 [SMILES: CC1(C)c2ccccc2-c2ccc3c4ccccc4n(-c4nc(-c5ccccc5)nc(-c5ccc6oc7ccccc7c6c5)n4)c3c21] | 79 |
| 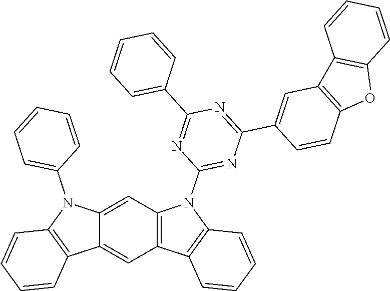 [SMILES: c1ccc(-c2nc(-c3ccc4oc5ccccc5c4c3)nc(-n3c4ccccc4c4cc5c6ccccc6n(-c6ccccc6)c5cc43)n2)cc1] | 80 |
| 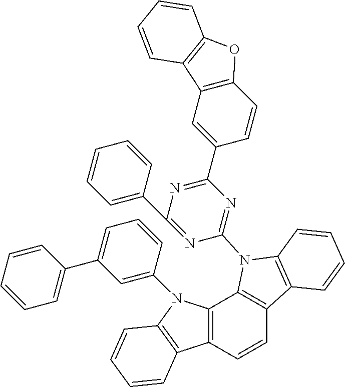 [SMILES: c1ccc(-c2cccc(-n3c4ccccc4c4ccc5c6ccccc6n(-c6nc(-c7ccccc7)nc(-c7ccc8oc9ccccc9c8c7)n6)c5c43)c2)cc1] | 81 |
| 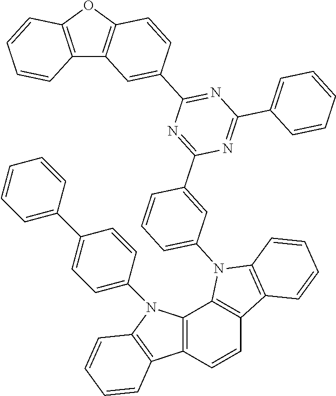 [SMILES: c1ccc(-c2ccc(-n3c4ccccc4c4ccc5c6ccccc6n(-c6cccc(-c7nc(-c8ccccc8)nc(-c8ccc9oc%10ccccc%10c9c8)n7)c6)c5c43)cc2)cc1] | 82 |
| 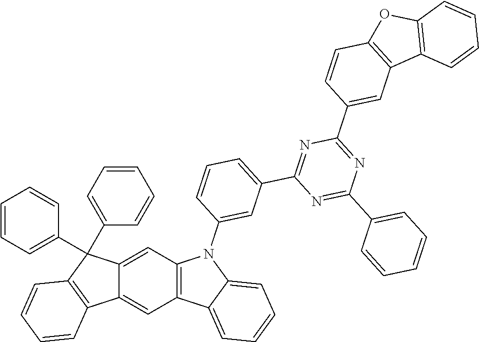 [SMILES: c1ccc(-c2nc(-c3cccc(-n4c5ccccc5c5cc6c(cc54)C(c4ccccc4)(c4ccccc4)c4ccccc4-6)c3)nc(-c3ccc4oc5ccccc5c4c3)n2)cc1] | 83 |
| 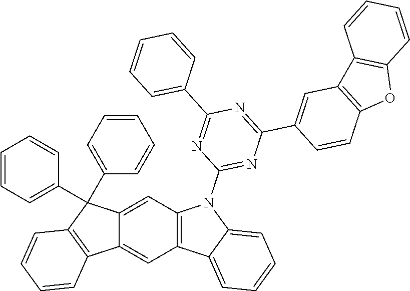 [SMILES: c1ccc(-c2nc(-c3ccc4oc5ccccc5c4c3)nc(-n3c4ccccc4c4cc5c(cc43)C(c3ccccc3)(c3ccccc3)c3ccccc3-5)n2)cc1] | 84 |
| 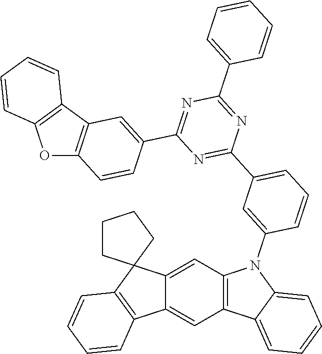 [SMILES: c1ccc(-c2nc(-c3cccc(-n4c5ccccc5c5cc6c(cc54)C4(CCCC4)c4ccccc4-6)c3)nc(-c3ccc4oc5ccccc5c4c3)n2)cc1] | 85 |
| 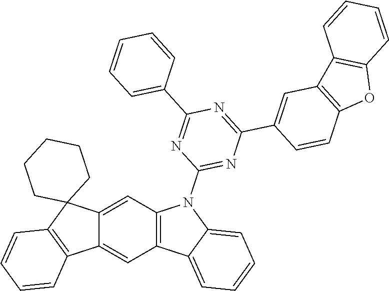 [SMILES: c1ccc(-c2nc(-c3ccc4oc5ccccc5c4c3)nc(-n3c4ccccc4c4cc5c(cc43)C3(CCCCC3)c3ccccc3-5)n2)cc1] | 86 |
| 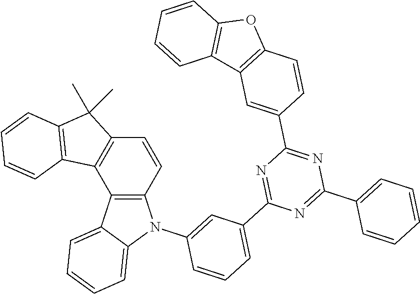 [SMILES: CC1(C)c2ccccc2-c2c1ccc1c2c2ccccc2n1-c1cccc(-c2nc(-c3ccccc3)nc(-c3ccc4oc5ccccc5c4c3)n2)c1] | 87 |
| 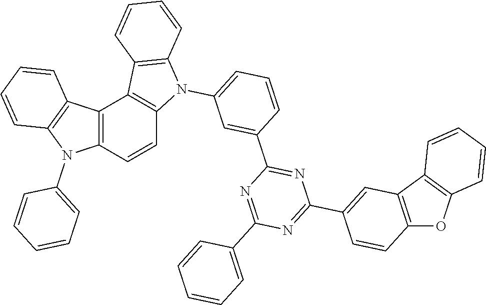 [SMILES: c1ccc(-c2nc(-c3cccc(-n4c5ccccc5c5c6c7ccccc7n(-c7ccccc7)c6ccc54)c3)nc(-c3ccc4oc5ccccc5c4c3)n2)cc1] | 88 |
| 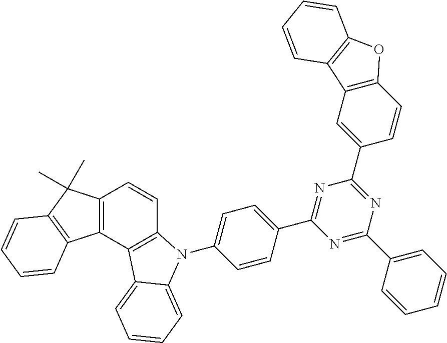 [SMILES: CC1(C)c2ccccc2-c2c1ccc1c2c2ccccc2n1-c1ccc(-c2nc(-c3ccccc3)nc(-c3ccc4oc5ccccc5c4c3)n2)cc1] | 89 |
| 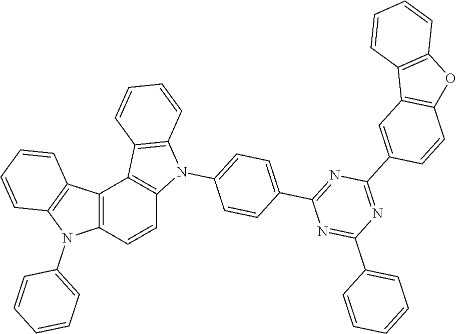 [SMILES: c1ccc(-c2nc(-c3ccc(-n4c5ccccc5c5c6c7ccccc7n(-c7ccccc7)c6ccc54)cc3)nc(-c3ccc4oc5ccccc5c4c3)n2)cc1] | 90 |
| 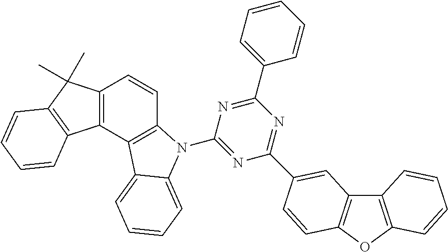 [SMILES: CC1(C)c2ccccc2-c2c1ccc1c2c2ccccc2n1-c1nc(-c2ccccc2)nc(-c2ccc3oc4ccccc4c3c2)n1] | 91 |
| 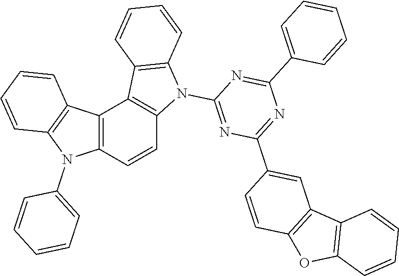 [SMILES: c1ccc(-c2nc(-c3ccc4oc5ccccc5c4c3)nc(-n3c4ccccc4c4c5c6ccccc6n(-c6ccccc6)c5ccc43)n2)cc1] | 92 |
| 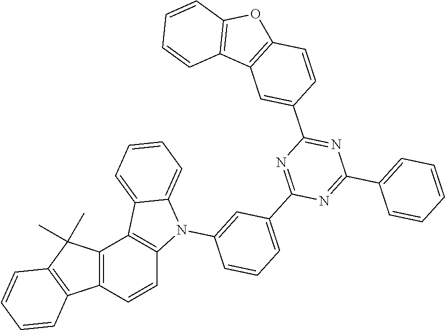 [SMILES: CC1(C)c2ccccc2-c2ccc3c(c21)c1ccccc1n3-c1cccc(-c2nc(-c3ccccc3)nc(-c3ccc4oc5ccccc5c4c3)n2)c1] | 93 |
| 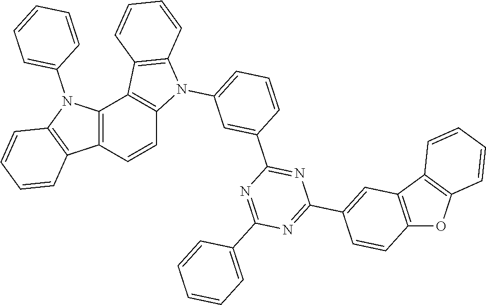 [SMILES: c1ccc(-c2nc(-c3cccc(-n4c5ccccc5c5c4ccc4c6ccccc6n(-c6ccccc6)c45)c3)nc(-c3ccc4oc5ccccc5c4c3)n2)cc1] | 94 |
| 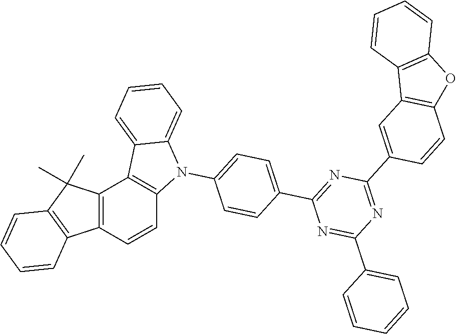 [SMILES: CC1(C)c2ccccc2-c2ccc3c(c21)c1ccccc1n3-c1ccc(-c2nc(-c3ccccc3)nc(-c3ccc4oc5ccccc5c4c3)n2)cc1] | 95 |
| 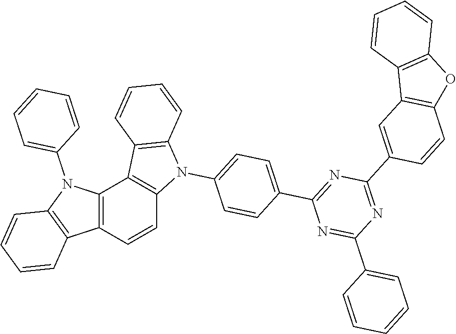 [SMILES: c1ccc(-c2nc(-c3ccc(-n4c5ccccc5c5c4ccc4c6ccccc6n(-c6ccccc6)c45)cc3)nc(-c3ccc4oc5ccccc5c4c3)n2)cc1] | 96 |
| 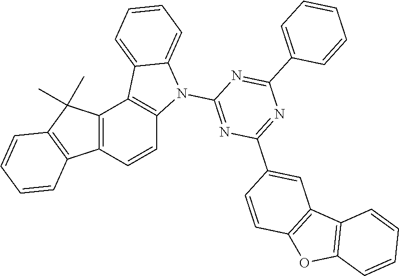 [SMILES: CC1(C)c2ccccc2-c2ccc3c(c21)c1ccccc1n3-c1nc(-c2ccccc2)nc(-c2ccc3oc4ccccc4c3c2)n1] | 97 |
| 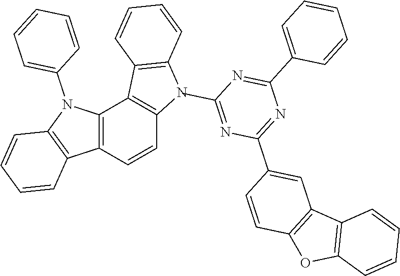 | 98 |
| 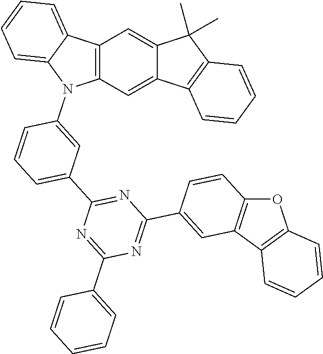 [SMILES: CC1(C)c2ccccc2-c2cc3c(cc21)c1ccccc1n3-c1cccc(-c2nc(-c3ccccc3)nc(-c3ccc4oc5ccccc5c4c3)n2)c1] | 99 |
| 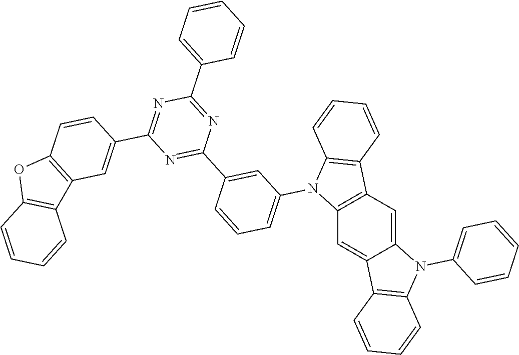 [SMILES: c1ccc(-c2nc(-c3cccc(-n4c5ccccc5c5cc6c(cc54)c4ccccc4n6-c4ccccc4)c3)nc(-c3ccc4oc5ccccc5c4c3)n2)cc1] | 100 |
| 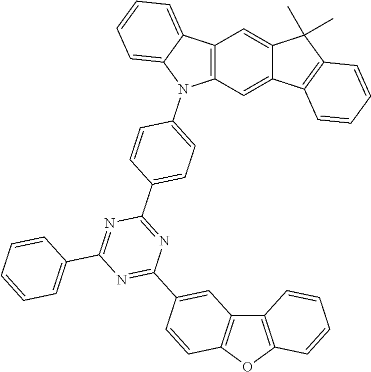 [SMILES: CC1(C)c2ccccc2-c2cc3c(cc21)c1ccccc1n3-c1ccc(-c2nc(-c3ccccc3)nc(-c3ccc4oc5ccccc5c4c3)n2)cc1] | 101 |
| 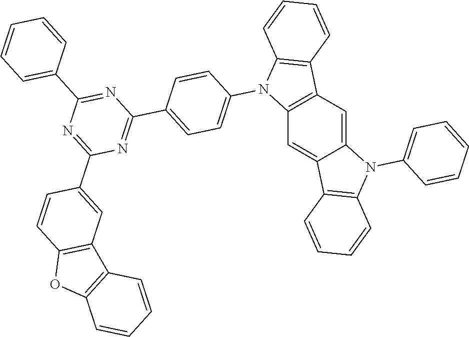 [SMILES: c1ccc(-c2nc(-c3ccc(-n4c5ccccc5c5cc6c(cc54)c4ccccc4n6-c4ccccc4)cc3)nc(-c3ccc4oc5ccccc5c4c3)n2)cc1] | 102 |
| 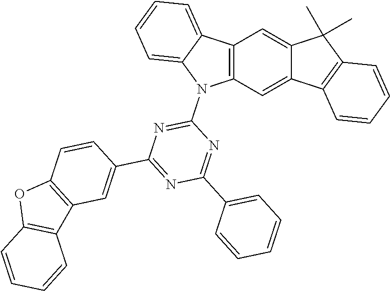 [SMILES: CC1(C)c2ccccc2-c2cc3c(cc21)c1ccccc1n3-c1nc(-c2ccccc2)nc(-c2ccc3oc4ccccc4c3c2)n1] | 103 |
| 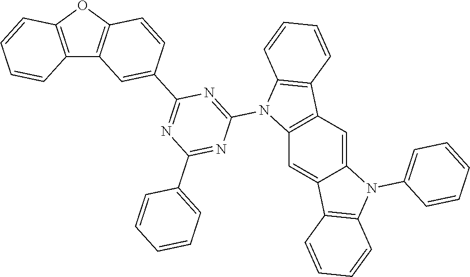 [SMILES: c1ccc(-c2nc(-c3ccc4oc5ccccc5c4c3)nc(-n3c4ccccc4c4cc5c(cc43)c3ccccc3n5-c3ccccc3)n2)cc1] | 104 |
| 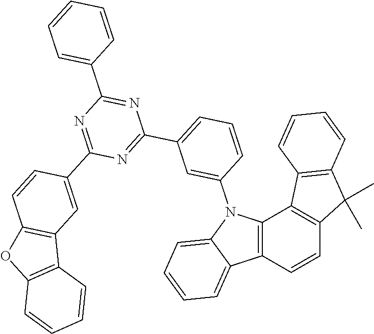 [SMILES: CC1(C)c2ccccc2-c2c1ccc1c3ccccc3n(-c3cccc(-c4nc(-c5ccccc5)nc(-c5ccc6oc7ccccc7c6c5)n4)c3)c21] | 105 |
| 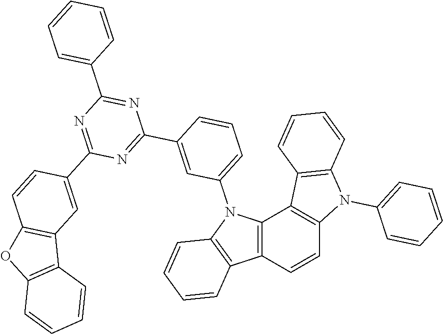 [SMILES: c1ccc(-c2nc(-c3cccc(-n4c5ccccc5c5ccc6c(c7ccccc7n6-c6ccccc6)c54)c3)nc(-c3ccc4oc5ccccc5c4c3)n2)cc1] | 106 |
| 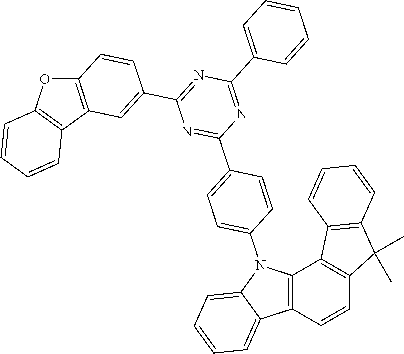 [SMILES: CC1(C)c2ccccc2-c2c1ccc1c3ccccc3n(-c3ccc(-c4nc(-c5ccccc5)nc(-c5ccc6oc7ccccc7c6c5)n4)cc3)c21] | 107 |
| 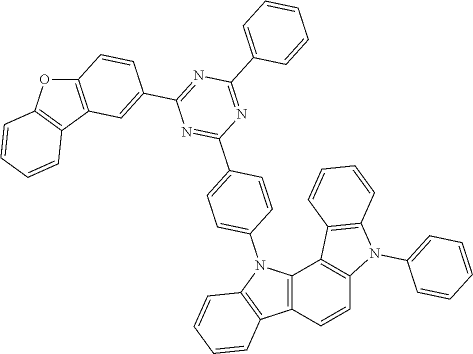 [SMILES: c1ccc(-c2nc(-c3ccc(-n4c5ccccc5c5ccc6c(c7ccccc7n6-c6ccccc6)c54)cc3)nc(-c3ccc4oc5ccccc5c4c3)n2)cc1] | 108 |
| 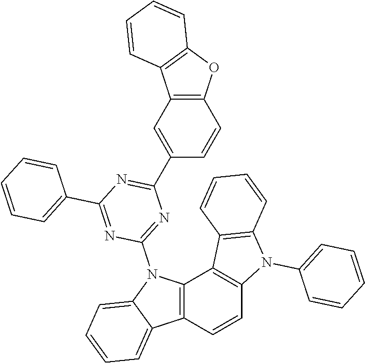 [SMILES: c1ccc(-c2nc(-c3ccc4oc5ccccc5c4c3)nc(-n3c4ccccc4c4ccc5c(c6ccccc6n5-c5ccccc5)c43)n2)cc1] | 109 |
| 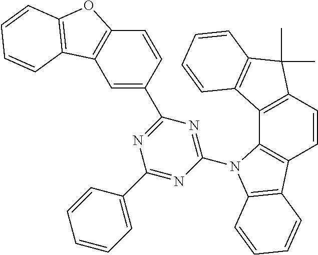 [SMILES: CC1(C)c2ccccc2-c2c1ccc1c3ccccc3n(-c3nc(-c4ccccc4)nc(-c4ccc5oc6ccccc6c5c4)n3)c21] | 110 |
| 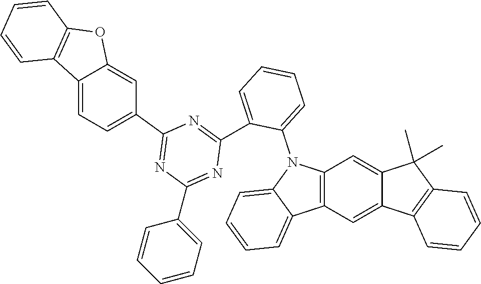 [SMILES: CC1(C)c2ccccc2-c2cc3c4ccccc4n(-c4ccccc4-c4nc(-c5ccccc5)nc(-c5ccc6c(c5)oc5ccccc56)n4)c3cc21] | 111 |
| 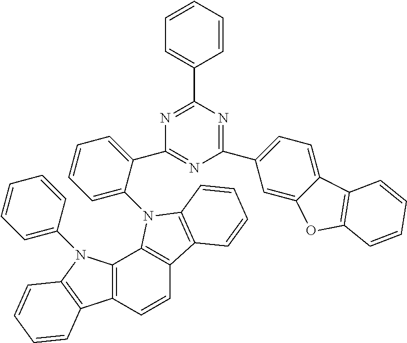 [SMILES: c1ccc(-c2nc(-c3ccc4c(c3)oc3ccccc34)nc(-c3ccccc3-n3c4ccccc4c4ccc5c6ccccc6n(-c6ccccc6)c5c43)n2)cc1] | 112 |
| 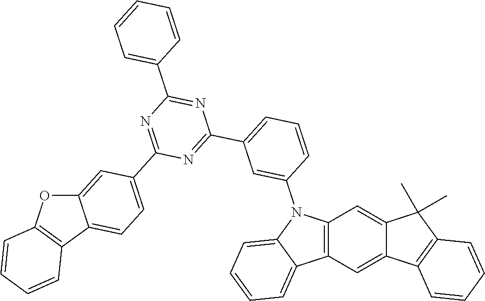 [SMILES: CC1(C)c2ccccc2-c2cc3c4ccccc4n(-c4cccc(-c5nc(-c6ccccc6)nc(-c6ccc7c(c6)oc6ccccc67)n5)c4)c3cc21] | 113 |
| 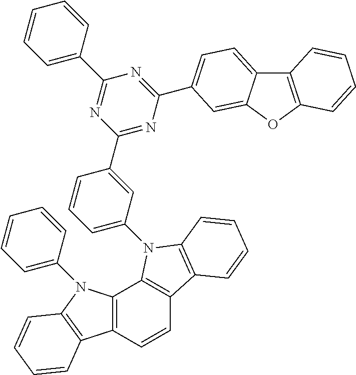 [SMILES: c1ccc(-c2nc(-c3cccc(-n4c5ccccc5c5ccc6c7ccccc7n(-c7ccccc7)c6c54)c3)nc(-c3ccc4c(c3)oc3ccccc34)n2)cc1] | 114 |
| 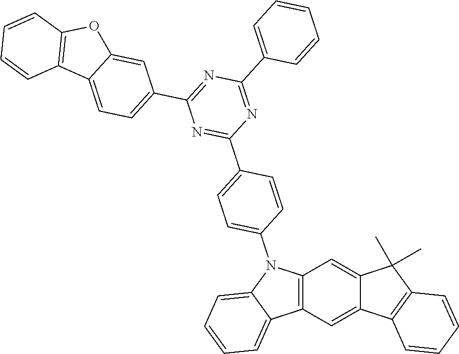 [SMILES: CC1(C)c2ccccc2-c2cc3c4ccccc4n(-c4ccc(-c5nc(-c6ccccc6)nc(-c6ccc7c(c6)oc6ccccc67)n5)cc4)c3cc21] | 115 |
| 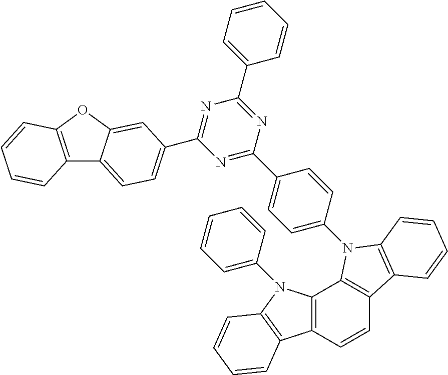 [SMILES: c1ccc(-c2nc(-c3ccc(-n4c5ccccc5c5ccc6c7ccccc7n(-c7ccccc7)c6c54)cc3)nc(-c3ccc4c(c3)oc3ccccc34)n2)cc1] | 116 |
| 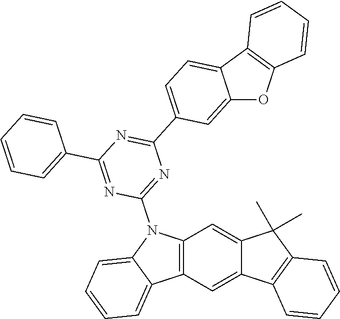 [SMILES: CC1(C)c2ccccc2-c2cc3c4ccccc4n(-c4nc(-c5ccccc5)nc(-c5ccc6c(c5)oc5ccccc56)n4)c3cc21] | 117 |
| 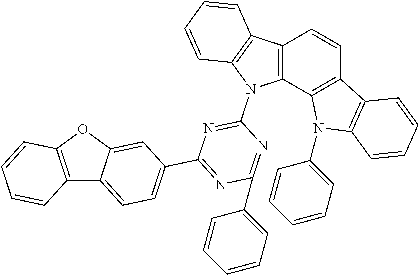 [SMILES: c1ccc(-c2nc(-c3ccc4c(c3)oc3ccccc34)nc(-n3c4ccccc4c4ccc5c6ccccc6n(-c6ccccc6)c5c43)n2)cc1] | 118 |
| 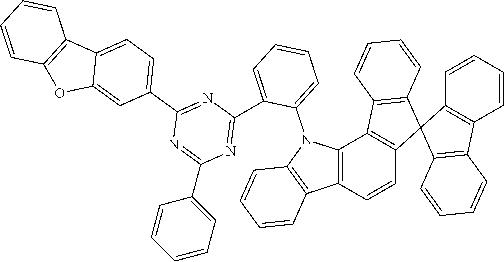 [SMILES: c1ccc(-c2nc(-c3ccc4c(c3)oc3ccccc34)nc(-c3ccccc3-n3c4ccccc4c4ccc5c(c43)-c3ccccc3C53c4ccccc4-c4ccccc43)n2)cc1] | 119 |
| 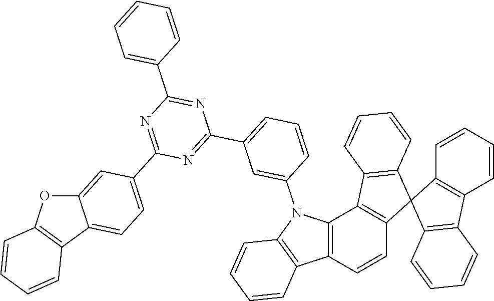 [SMILES: c1ccc(-c2nc(-c3cccc(-n4c5ccccc5c5ccc6c(c54)-c4ccccc4C64c5ccccc5-c5ccccc54)c3)nc(-c3ccc4c(c3)oc3ccccc34)n2)cc1] | 120 |
| 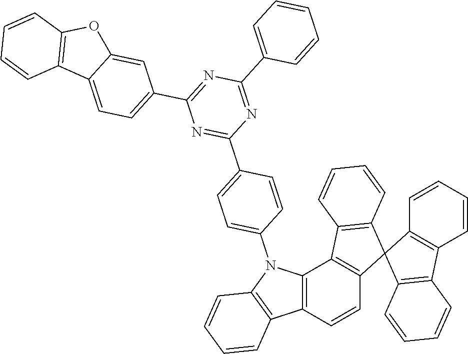 [SMILES: c1ccc(-c2nc(-c3ccc(-n4c5ccccc5c5ccc6c(c54)-c4ccccc4C64c5ccccc5-c5ccccc54)cc3)nc(-c3ccc4c(c3)oc3ccccc34)n2)cc1] | 121 |
| 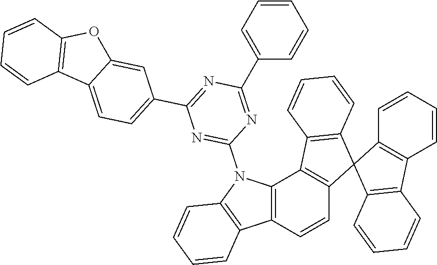 [SMILES: c1ccc(-c2nc(-c3ccc4c(c3)oc3ccccc34)nc(-n3c4ccccc4c4ccc5c(c43)-c3ccccc3C53c4ccccc4-c4ccccc43)n2)cc1] | 122 |
| 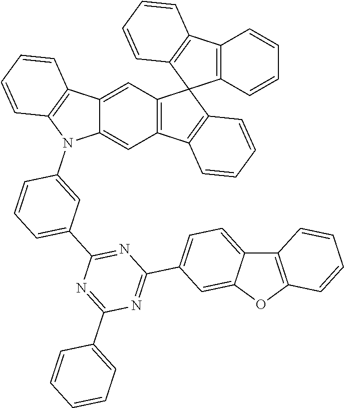 [SMILES: c1ccc(-c2nc(-c3cccc(-n4c5ccccc5c5cc6c(cc54)-c4ccccc4C64c5ccccc5-c5ccccc54)c3)nc(-c3ccc4c(c3)oc3ccccc34)n2)cc1] | 123 |
| 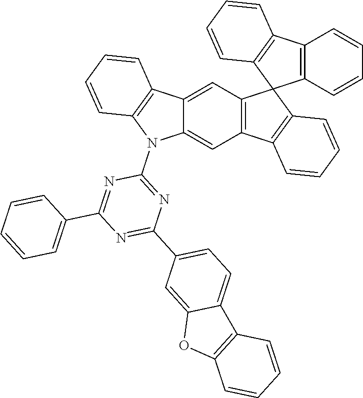 [SMILES: c1ccc(-c2nc(-c3ccc4c(c3)oc3ccccc34)nc(-n3c4ccccc4c4cc5c(cc43)-c3ccccc3C53c4ccccc4-c4ccccc43)n2)cc1] | 124 |
| 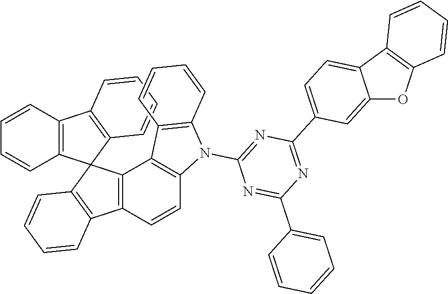 [SMILES: c1ccc(-c2nc(-c3ccc4c(c3)oc3ccccc34)nc(-n3c4ccccc4c4c5c(ccc43)-c3ccccc3C53c4ccccc4-c4ccccc43)n2)cc1] | 125 |
| 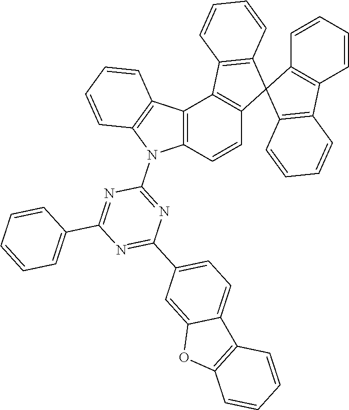 [SMILES: c1ccc(-c2nc(-c3ccc4c(c3)oc3ccccc34)nc(-n3c4ccccc4c4c5c(ccc43)C3(c4ccccc4-c4ccccc43)c3ccccc3-5)n2)cc1] | 126 |
| 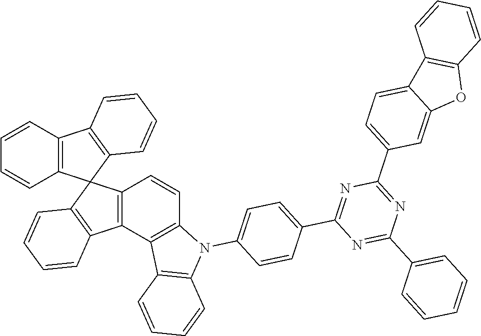 [SMILES: c1ccc(-c2nc(-c3ccc(-n4c5ccccc5c5c6c(ccc54)C4(c5ccccc5-c5ccccc54)c4ccccc4-6)cc3)nc(-c3ccc4c(c3)oc3ccccc34)n2)cc1] | 127 |
| 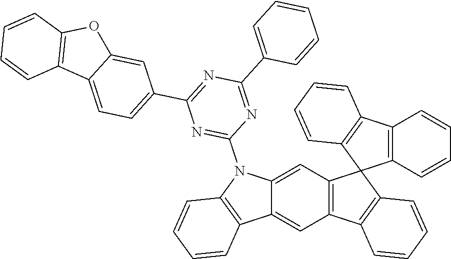 [SMILES: c1ccc(-c2nc(-c3ccc4c(c3)oc3ccccc34)nc(-n3c4ccccc4c4cc5c(cc43)C3(c4ccccc4-c4ccccc43)c3ccccc3-5)n2)cc1] | 128 |
| 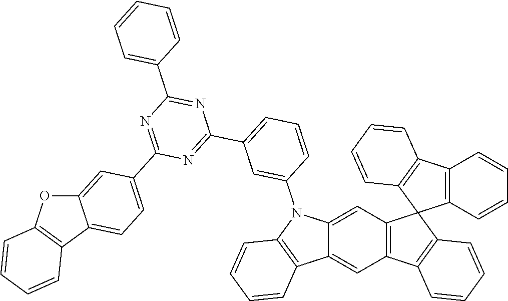 [SMILES: c1ccc(-c2nc(-c3cccc(-n4c5ccccc5c5cc6c(cc54)C4(c5ccccc5-c5ccccc54)c4ccccc4-6)c3)nc(-c3ccc4c(c3)oc3ccccc34)n2)cc1] | 129 |
| 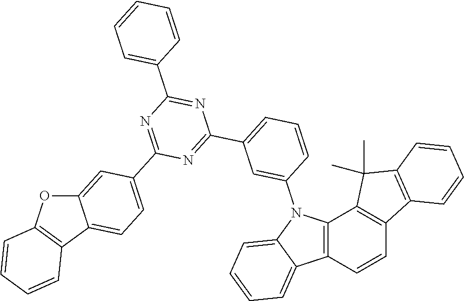 [SMILES: CC1(C)c2ccccc2-c2ccc3c4ccccc4n(-c4cccc(-c5nc(-c6ccccc6)nc(-c6ccc7c(c6)oc6ccccc67)n5)c4)c3c21] | 130 |
| 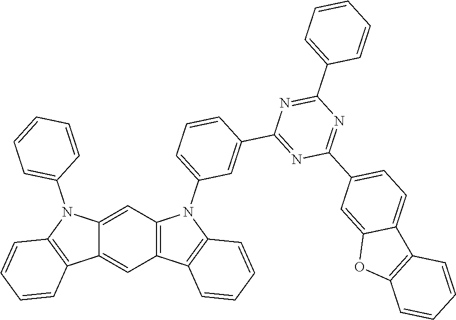 [SMILES: c1ccc(-c2nc(-c3cccc(-n4c5ccccc5c5cc6c7ccccc7n(-c7ccccc7)c6cc54)c3)nc(-c3ccc4c(c3)oc3ccccc34)n2)cc1] | 131 |
| 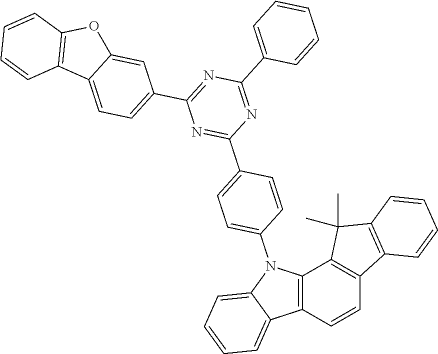 [SMILES: CC1(C)c2ccccc2-c2ccc3c4ccccc4n(-c4ccc(-c5nc(-c6ccccc6)nc(-c6ccc7c(c6)oc6ccccc67)n5)cc4)c3c21] | 132 |
| 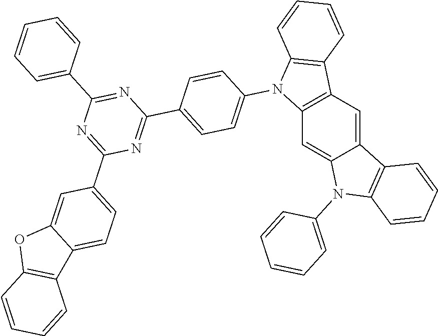 [SMILES: c1ccc(-c2nc(-c3ccc(-n4c5ccccc5c5cc6c7ccccc7n(-c7ccccc7)c6cc54)cc3)nc(-c3ccc4c(c3)oc3ccccc34)n2)cc1] | 133 |
| 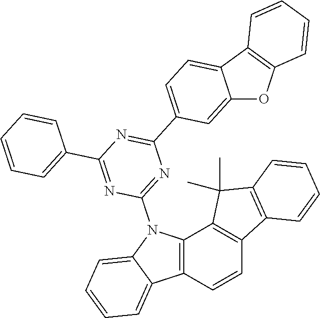 [SMILES: CC1(C)c2ccccc2-c2ccc3c4ccccc4n(-c4nc(-c5ccccc5)nc(-c5ccc6c(c5)oc5ccccc56)n4)c3c21] | 134 |
| 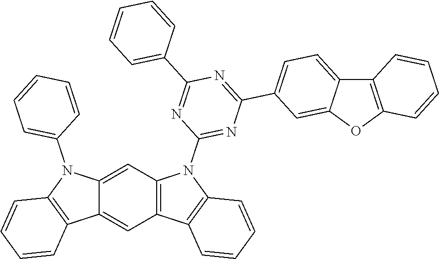 [SMILES: c1ccc(-c2nc(-c3ccc4c(c3)oc3ccccc34)nc(-n3c4ccccc4c4cc5c6ccccc6n(-c6ccccc6)c5cc43)n2)cc1] | 135 |
| 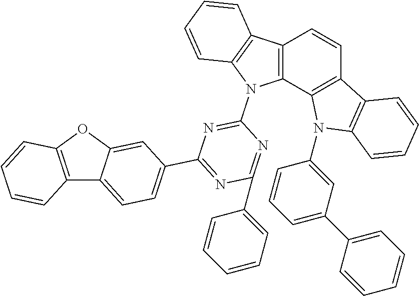 [SMILES: c1ccc(-c2cccc(-n3c4ccccc4c4ccc5c6ccccc6n(-c6nc(-c7ccccc7)nc(-c7ccc8c(c7)oc7ccccc78)n6)c5c43)c2)cc1] | 136 |
| 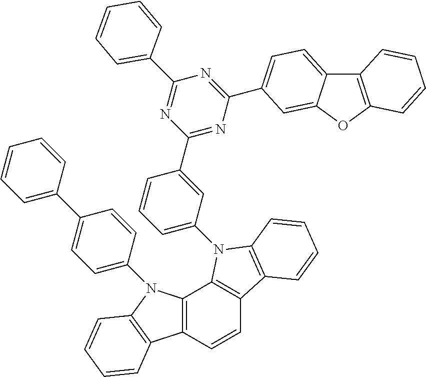 [SMILES: c1ccc(-c2ccc(-n3c4ccccc4c4ccc5c6ccccc6n(-c6cccc(-c7nc(-c8ccccc8)nc(-c8ccc9c(c8)oc8ccccc89)n7)c6)c5c43)cc2)cc1] | 137 |
| 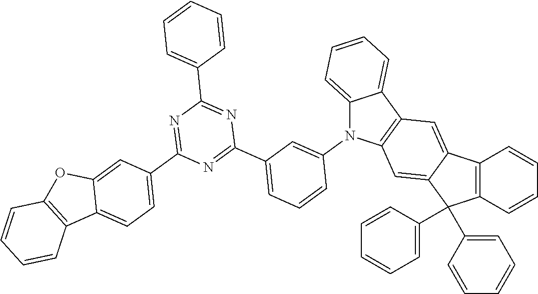 [SMILES: c1ccc(-c2nc(-c3cccc(-n4c5ccccc5c5cc6c(cc54)C(c4ccccc4)(c4ccccc4)c4ccccc4-6)c3)nc(-c3ccc4c(c3)oc3ccccc34)n2)cc1] | 138 |
| 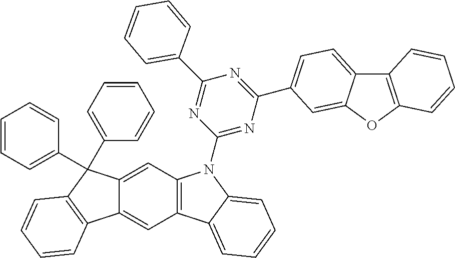 [SMILES: c1ccc(-c2nc(-c3ccc4c(c3)oc3ccccc34)nc(-n3c4ccccc4c4cc5c(cc43)C(c3ccccc3)(c3ccccc3)c3ccccc3-5)n2)cc1] | 139 |
| 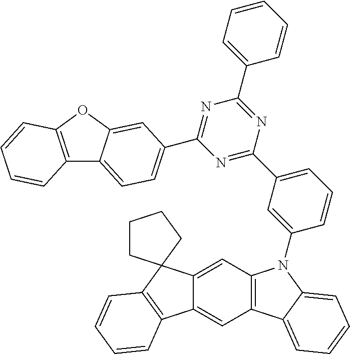 [SMILES: c1ccc(-c2nc(-c3cccc(-n4c5ccccc5c5cc6c(cc54)C4(CCCC4)c4ccccc4-6)c3)nc(-c3ccc4c(c3)oc3ccccc34)n2)cc1] | 140 |
| 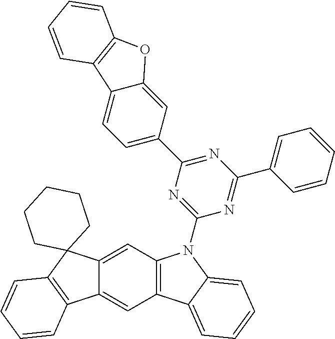 [SMILES: c1ccc(-c2nc(-c3ccc4c(c3)oc3ccccc34)nc(-n3c4ccccc4c4cc5c(cc43)C3(CCCCC3)c3ccccc3-5)n2)cc1] | 141 |
| 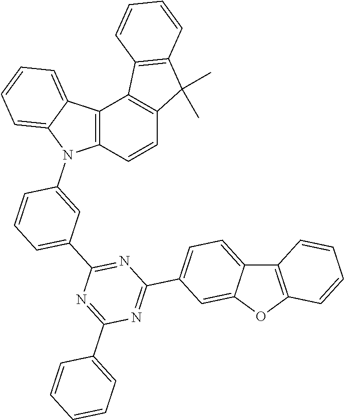 [SMILES: CC1(C)c2ccccc2-c2c1ccc1c2c2ccccc2n1-c1cccc(-c2nc(-c3ccccc3)nc(-c3ccc4c(c3)oc3ccccc34)n2)c1] | 142 |
| 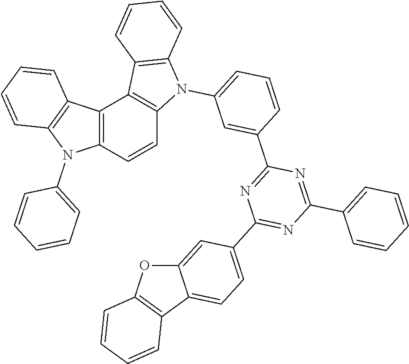 [SMILES: c1ccc(-c2nc(-c3cccc(-n4c5ccccc5c5c6c7ccccc7n(-c7ccccc7)c6ccc54)c3)nc(-c3ccc4c(c3)oc3ccccc34)n2)cc1] | 143 |
| 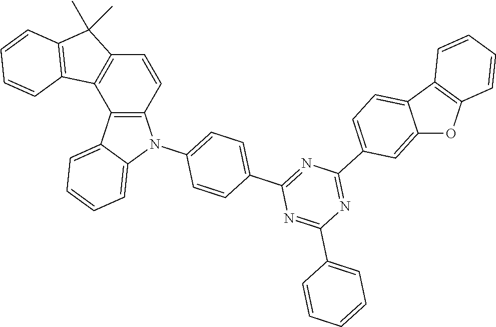 [SMILES: CC1(C)c2ccccc2-c2c1ccc1c2c2ccccc2n1-c1ccc(-c2nc(-c3ccccc3)nc(-c3ccc4c(c3)oc3ccccc34)n2)cc1] | 144 |
| 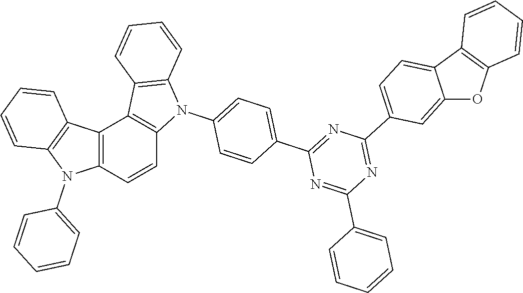 [SMILES: c1ccc(-c2nc(-c3ccc(-n4c5ccccc5c5c6c7ccccc7n(-c7ccccc7)c6ccc54)cc3)nc(-c3ccc4c(c3)oc3ccccc34)n2)cc1] | 145 |
| 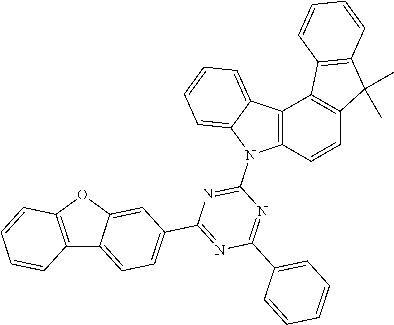 [SMILES: CC1(C)c2ccccc2-c2c1ccc1c2c2ccccc2n1-c1nc(-c2ccccc2)nc(-c2ccc3c(c2)oc2ccccc23)n1] | 146 |
| 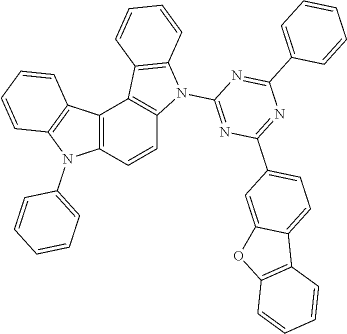 [SMILES: c1ccc(-c2nc(-c3ccc4c(c3)oc3ccccc34)nc(-n3c4ccccc4c4c5c6ccccc6n(-c6ccccc6)c5ccc43)n2)cc1] | 147 |
| 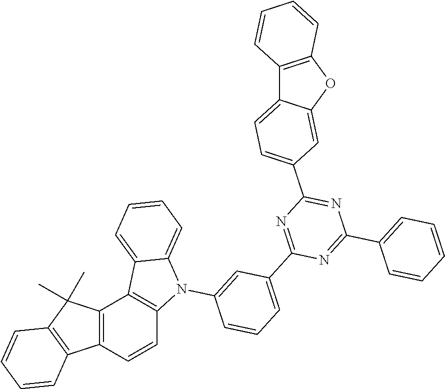 [SMILES: CC1(C)c2ccccc2-c2ccc3c(c21)c1ccccc1n3-c1cccc(-c2nc(-c3ccccc3)nc(-c3ccc4c(c3)oc3ccccc34)n2)c1] | 148 |
| 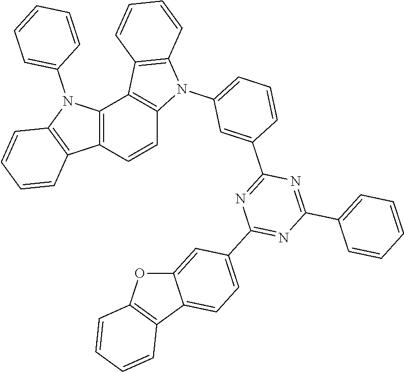 [SMILES: c1ccc(-c2nc(-c3cccc(-n4c5ccccc5c5c4ccc4c6ccccc6n(-c6ccccc6)c45)c3)nc(-c3ccc4c(c3)oc3ccccc34)n2)cc1] | 149 |
| 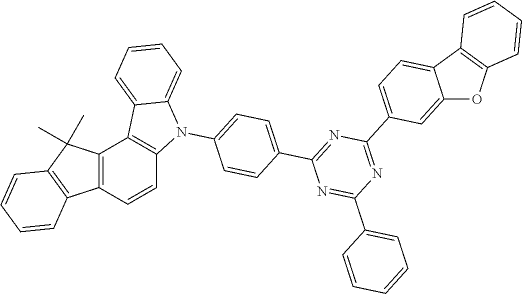 [SMILES: CC1(C)c2ccccc2-c2ccc3c(c21)c1ccccc1n3-c1ccc(-c2nc(-c3ccccc3)nc(-c3ccc4c(c3)oc3ccccc34)n2)cc1] | 150 |
| 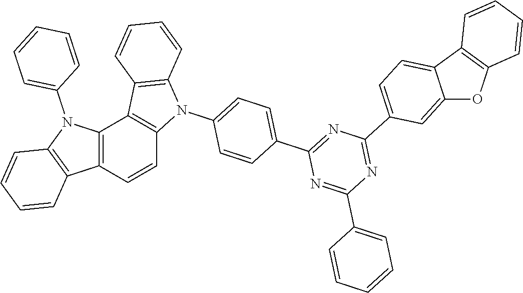 [SMILES: c1ccc(-c2nc(-c3ccc(-n4c5ccccc5c5c4ccc4c6ccccc6n(-c6ccccc6)c45)cc3)nc(-c3ccc4c(c3)oc3ccccc34)n2)cc1] | 151 |
| 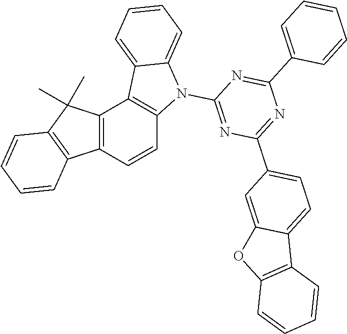 [SMILES: CC1(C)c2ccccc2-c2ccc3c(c21)c1ccccc1n3-c1nc(-c2ccccc2)nc(-c2ccc3c(c2)oc2ccccc23)n1] | 152 |
| 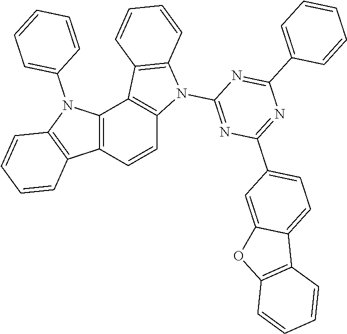 [SMILES: c1ccc(-c2nc(-c3ccc4c(c3)oc3ccccc34)nc(-n3c4ccccc4c4c3ccc3c5ccccc5n(-c5ccccc5)c34)n2)cc1] | 153 |
| 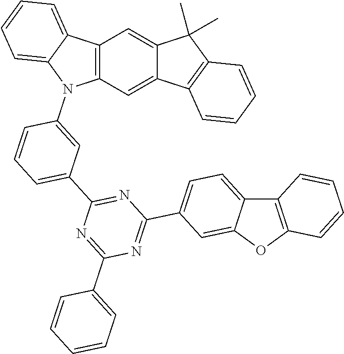 [SMILES: CC1(C)c2ccccc2-c2cc3c(cc21)c1ccccc1n3-c1cccc(-c2nc(-c3ccccc3)nc(-c3ccc4c(c3)oc3ccccc34)n2)c1] | 154 |
| 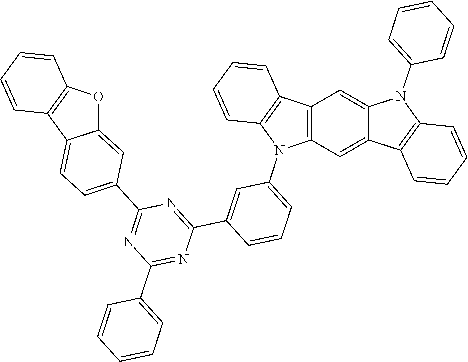 [SMILES: c1ccc(-c2nc(-c3cccc(-n4c5ccccc5c5cc6c(cc54)c4ccccc4n6-c4ccccc4)c3)nc(-c3ccc4c(c3)oc3ccccc34)n2)cc1] | 155 |
| 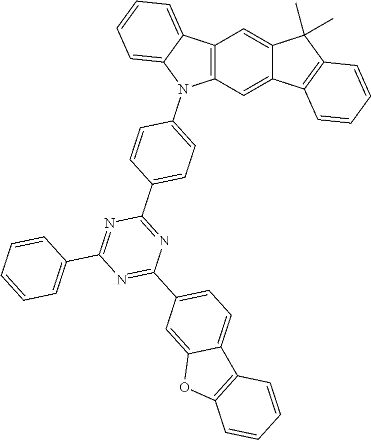 [SMILES: CC1(C)c2ccccc2-c2cc3c(cc21)c1ccccc1n3-c1ccc(-c2nc(-c3ccccc3)nc(-c3ccc4c(c3)oc3ccccc34)n2)cc1] | 156 |
| 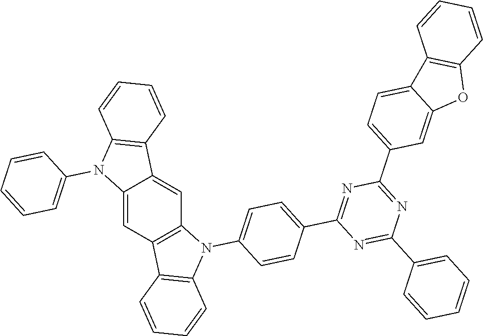 [SMILES: c1ccc(-c2nc(-c3ccc(-n4c5ccccc5c5cc6c(cc54)c4ccccc4n6-c4ccccc4)cc3)nc(-c3ccc4c(c3)oc3ccccc34)n2)cc1] | 157 |
| 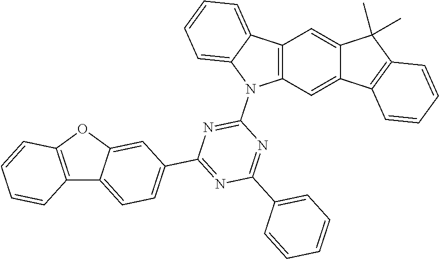 [SMILES: CC1(C)c2ccccc2-c2cc3c(cc21)c1ccccc1n3-c1nc(-c2ccccc2)nc(-c2ccc3c(c2)oc2ccccc23)n1] | 158 |
| 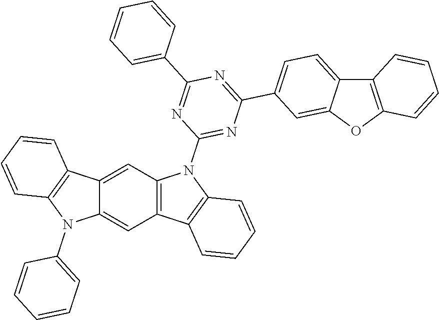 [SMILES: c1ccc(-c2nc(-c3ccc4c(c3)oc3ccccc34)nc(-n3c4ccccc4c4cc5c(cc43)c3ccccc3n5-c3ccccc3)n2)cc1] | 159 |
| 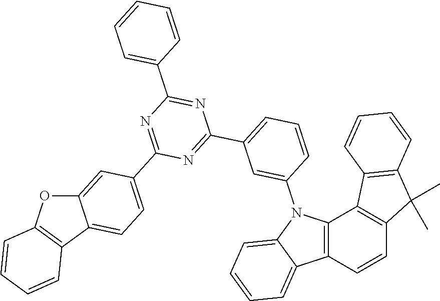 [SMILES: CC1(C)c2ccccc2-c2c1ccc1c3ccccc3n(-c3cccc(-c4nc(-c5ccccc5)nc(-c5ccc6c(c5)oc5ccccc56)n4)c3)c21] | 160 |
| 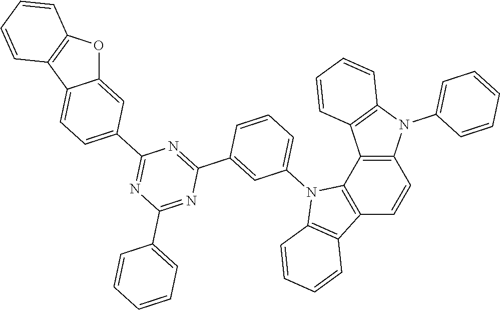 [SMILES: c1ccc(-c2nc(-c3cccc(-n4c5ccccc5c5ccc6c(c7ccccc7n6-c6ccccc6)c54)c3)nc(-c3ccc4c(c3)oc3ccccc34)n2)cc1] | 161 |
| 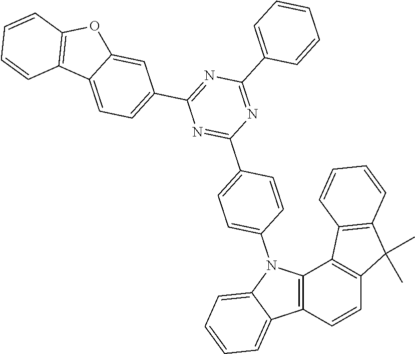 [SMILES: CC1(C)c2ccccc2-c2c1ccc1c3ccccc3n(-c3ccc(-c4nc(-c5ccccc5)nc(-c5ccc6c(c5)oc5ccccc56)n4)cc3)c21] | 162 |
| 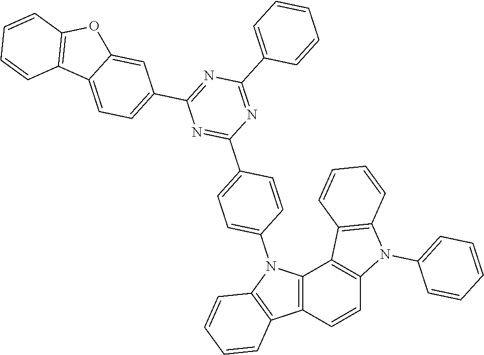 [SMILES: c1ccc(-c2nc(-c3ccc(-n4c5ccccc5c5ccc6c(c7ccccc7n6-c6ccccc6)c54)cc3)nc(-c3ccc4c(c3)oc3ccccc34)n2)cc1] | 163 |
| 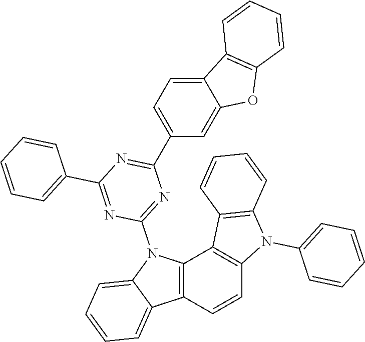 [SMILES: c1ccc(-c2nc(-c3ccc4c(c3)oc3ccccc34)nc(-n3c4ccccc4c4ccc5c(c6ccccc6n5-c5ccccc5)c43)n2)cc1] | 164 |
| 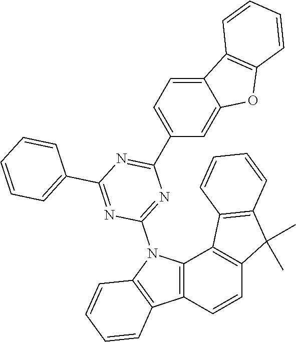 [SMILES: CC1(C)c2ccccc2-c2c1ccc1c3ccccc3n(-c3nc(-c4ccccc4)nc(-c4ccc5c(c4)oc4ccccc45)n3)c21] | 165 |
| 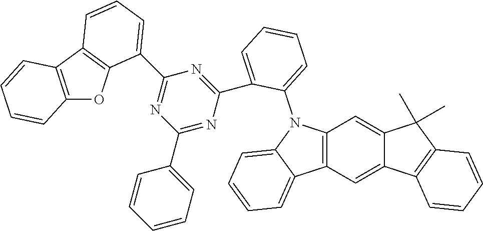 [SMILES: CC1(C)c2ccccc2-c2cc3c4ccccc4n(-c4ccccc4-c4nc(-c5ccccc5)nc(-c5cccc6c5oc5ccccc56)n4)c3cc21] | 166 |
| 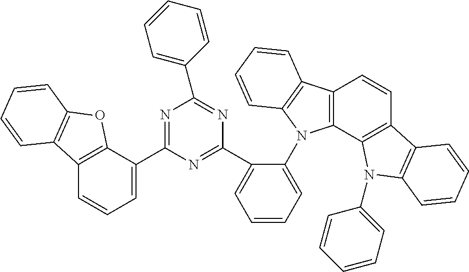 [SMILES: c1ccc(-c2nc(-c3ccccc3-n3c4ccccc4c4ccc5c6ccccc6n(-c6ccccc6)c5c43)nc(-c3cccc4c3oc3ccccc34)n2)cc1] | 167 |
| 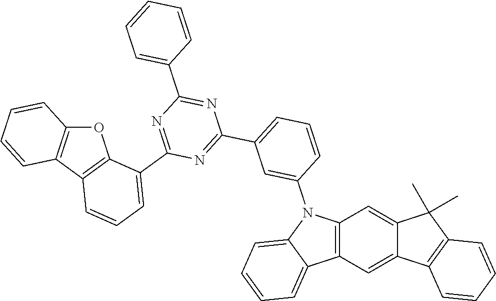 [SMILES: CC1(C)c2ccccc2-c2cc3c4ccccc4n(-c4cccc(-c5nc(-c6ccccc6)nc(-c6cccc7c6oc6ccccc67)n5)c4)c3cc21] | 168 |
| 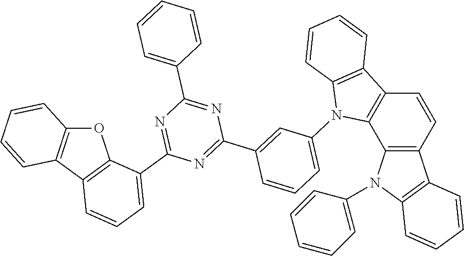 | 169 |
| 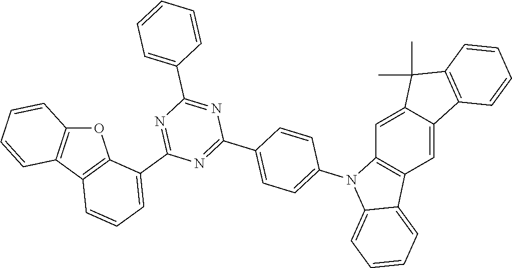 | 170 |
| 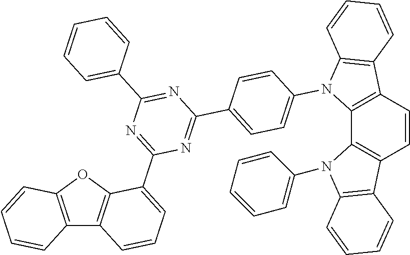 [SMILES: c1ccc(-c2nc(-c3ccc(-n4c5ccccc5c5ccc6c7ccccc7n(-c7ccccc7)c6c54)cc3)nc(-c3cccc4c3oc3ccccc34)n2)cc1] | 171 |
| 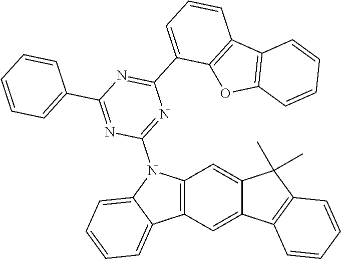 [SMILES: CC1(C)c2ccccc2-c2cc3c4ccccc4n(-c4nc(-c5ccccc5)nc(-c5cccc6c5oc5ccccc56)n4)c3cc21] | 172 |
| 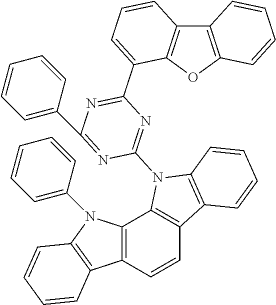 [SMILES: c1ccc(-c2nc(-c3cccc4c3oc3ccccc34)nc(-n3c4ccccc4c4ccc5c6ccccc6n(-c6ccccc6)c5c43)n2)cc1] | 173 |
| 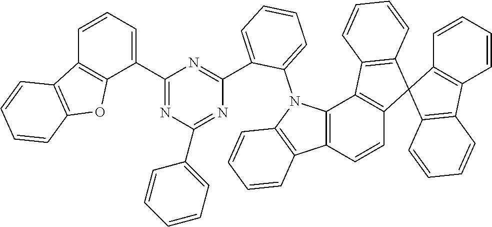 [SMILES: c1ccc(-c2nc(-c3ccccc3-n3c4ccccc4c4ccc5c(c43)-c3ccccc3C53c4ccccc4-c4ccccc43)nc(-c3cccc4c3oc3ccccc34)n2)cc1] | 174 |
| 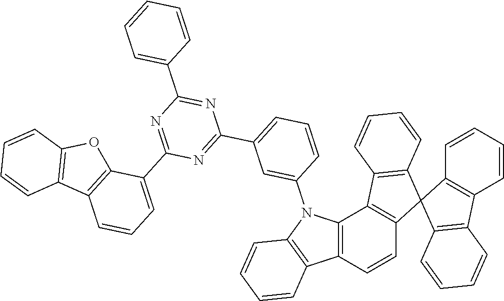 [SMILES: c1ccc(-c2nc(-c3cccc(-n4c5ccccc5c5ccc6c(c54)-c4ccccc4C64c5ccccc5-c5ccccc54)c3)nc(-c3cccc4c3oc3ccccc34)n2)cc1] | 175 |
| 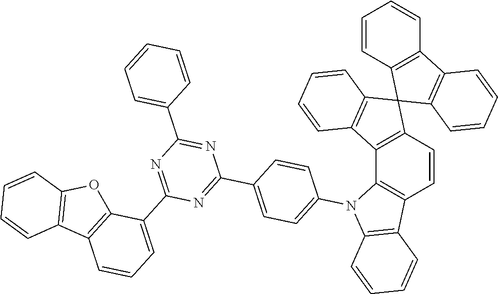 [SMILES: c1ccc(-c2nc(-c3ccc(-n4c5ccccc5c5ccc6c(c54)-c4ccccc4C64c5ccccc5-c5ccccc54)cc3)nc(-c3cccc4c3oc3ccccc34)n2)cc1] | 176 |
| 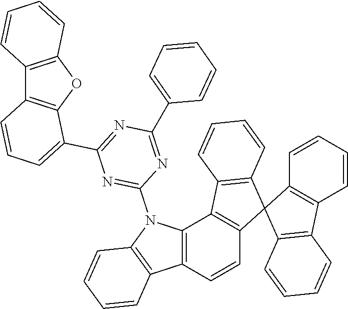 [SMILES: c1ccc(-c2nc(-c3cccc4c3oc3ccccc34)nc(-n3c4ccccc4c4ccc5c(c43)-c3ccccc3C53c4ccccc4-c4ccccc43)n2)cc1] | 177 |
| 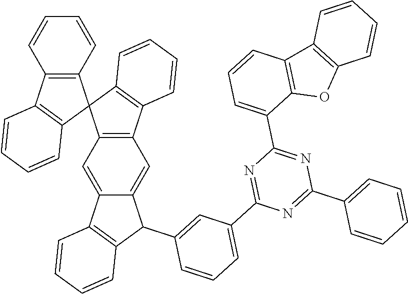 [SMILES: c1ccc(-c2nc(-c3cccc(C4c5ccccc5-c5cc6c(cc54)-c4ccccc4C64c5ccccc5-c5ccccc54)c3)nc(-c3cccc4c3oc3ccccc34)n2)cc1] | 178 |
| 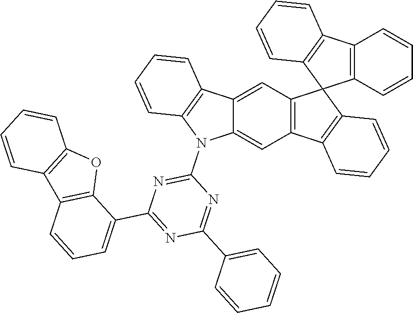 [SMILES: c1ccc(-c2nc(-c3cccc4c3oc3ccccc34)nc(-n3c4ccccc4c4cc5c(cc43)-c3ccccc3C53c4ccccc4-c4ccccc43)n2)cc1] | 179 |
| 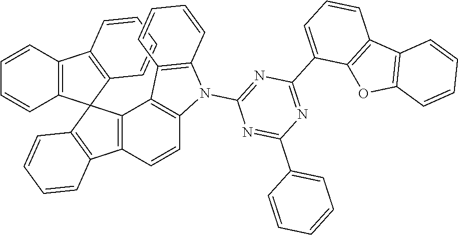 [SMILES: c1ccc(-c2nc(-c3cccc4c3oc3ccccc34)nc(-n3c4ccccc4c4c5c(ccc43)-c3ccccc3C53c4ccccc4-c4ccccc43)n2)cc1] | 180 |
| 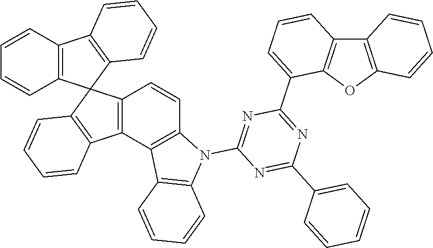 [SMILES: c1ccc(-c2nc(-c3cccc4c3oc3ccccc34)nc(-n3c4ccccc4c4c5c(ccc43)C3(c4ccccc4-c4ccccc43)c3ccccc3-5)n2)cc1] | 181 |
| 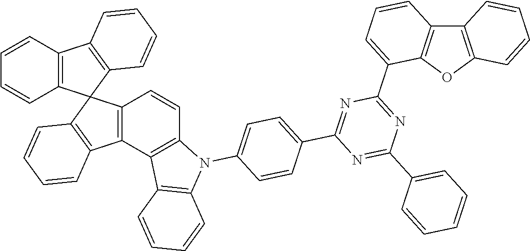 [SMILES: c1ccc(-c2nc(-c3ccc(-n4c5ccccc5c5c6c(ccc54)C4(c5ccccc5-c5ccccc54)c4ccccc4-6)cc3)nc(-c3cccc4c3oc3ccccc34)n2)cc1] | 182 |
| 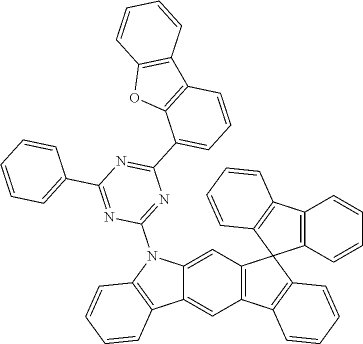 [SMILES: c1ccc(-c2nc(-c3cccc4c3oc3ccccc34)nc(-n3c4ccccc4c4cc5c(cc43)C3(c4ccccc4-c4ccccc43)c3ccccc3-5)n2)cc1] | 183 |
| 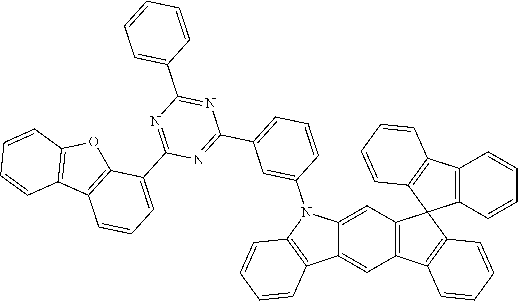 [SMILES: c1ccc(-c2nc(-c3cccc(-n4c5ccccc5c5cc6c(cc54)C4(c5ccccc5-c5ccccc54)c4ccccc4-6)c3)nc(-c3cccc4c3oc3ccccc34)n2)cc1] | 184 |
| 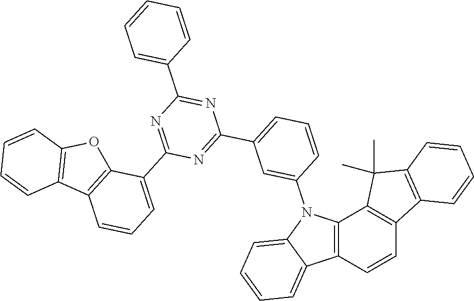 [SMILES: CC1(C)c2ccccc2-c2ccc3c4ccccc4n(-c4cccc(-c5nc(-c6ccccc6)nc(-c6cccc7c6oc6ccccc67)n5)c4)c3c21] | 185 |
| 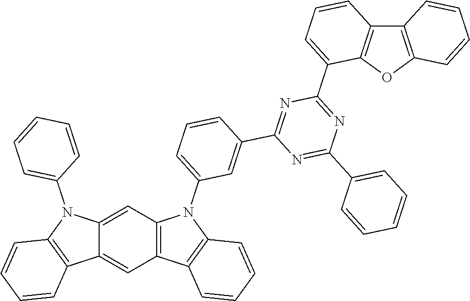 [SMILES: c1ccc(-c2nc(-c3cccc(-n4c5ccccc5c5cc6c7ccccc7n(-c7ccccc7)c6cc54)c3)nc(-c3cccc4c3oc3ccccc34)n2)cc1] | 186 |
| 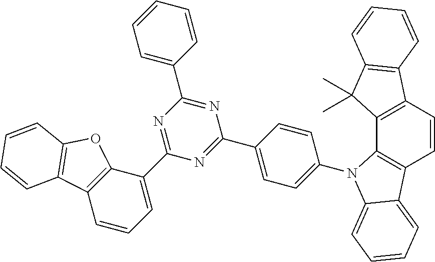 [SMILES: CC1(C)c2ccccc2-c2ccc3c4ccccc4n(-c4ccc(-c5nc(-c6ccccc6)nc(-c6cccc7c6oc6ccccc67)n5)cc4)c3c21] | 187 |
| 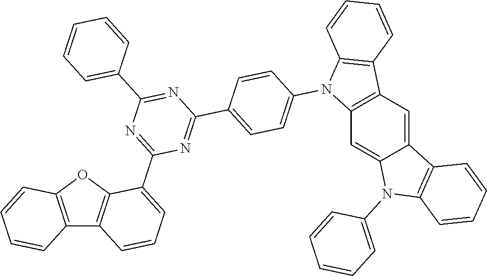 [SMILES: c1ccc(-c2nc(-c3ccc(-n4c5ccccc5c5cc6c7ccccc7n(-c7ccccc7)c6cc54)cc3)nc(-c3cccc4c3oc3ccccc34)n2)cc1] | 188 |
| 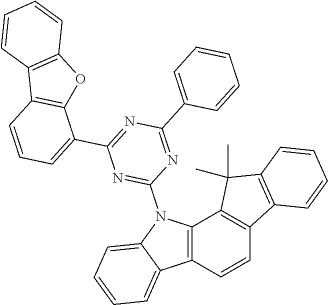 [SMILES: CC1(C)c2ccccc2-c2ccc3c4ccccc4n(-c4nc(-c5ccccc5)nc(-c5cccc6c5oc5ccccc56)n4)c3c21] | 189 |
| 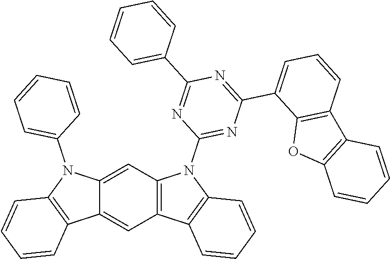 [SMILES: c1ccc(-c2nc(-c3cccc4c3oc3ccccc34)nc(-n3c4ccccc4c4cc5c6ccccc6n(-c6ccccc6)c5cc43)n2)cc1] | 190 |
| 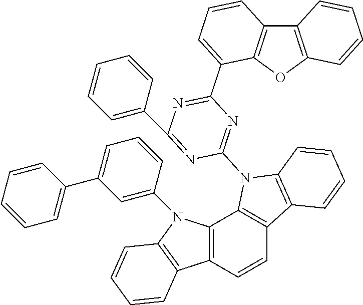 [SMILES: c1ccc(-c2cccc(-n3c4ccccc4c4ccc5c6ccccc6n(-c6nc(-c7ccccc7)nc(-c7cccc8c7oc7ccccc78)n6)c5c43)c2)cc1] | 191 |
| 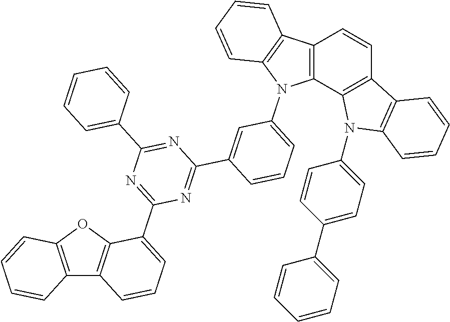 [SMILES: c1ccc(-c2ccc(-n3c4ccccc4c4ccc5c6ccccc6n(-c6cccc(-c7nc(-c8ccccc8)nc(-c8cccc9c8oc8ccccc89)n7)c6)c5c43)cc2)cc1] | 192 |
| 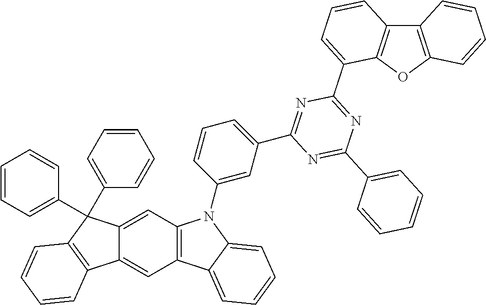 [SMILES: c1ccc(-c2nc(-c3cccc(-n4c5ccccc5c5cc6c(cc54)C(c4ccccc4)(c4ccccc4)c4ccccc4-6)c3)nc(-c3cccc4c3oc3ccccc34)n2)cc1] | 193 |
| 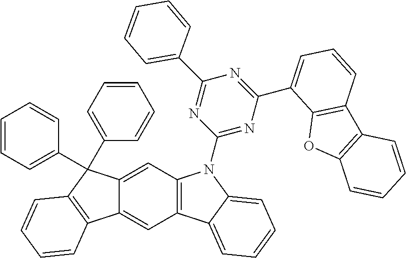 [SMILES: c1ccc(-c2nc(-c3cccc4c3oc3ccccc34)nc(-n3c4ccccc4c4cc5c(cc43)C(c3ccccc3)(c3ccccc3)c3ccccc3-5)n2)cc1] | 194 |
| 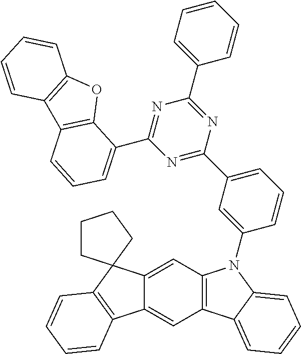 [SMILES: c1ccc(-c2nc(-c3cccc(-n4c5ccccc5c5cc6c(cc54)C4(CCCC4)c4ccccc4-6)c3)nc(-c3cccc4c3oc3ccccc34)n2)cc1] | 195 |
| 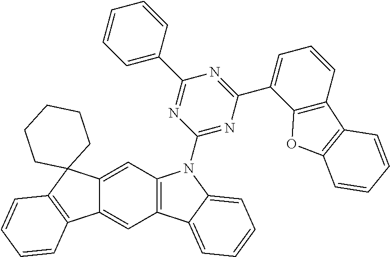 [SMILES: c1ccc(-c2nc(-c3cccc4c3oc3ccccc34)nc(-n3c4ccccc4c4cc5c(cc43)C3(CCCCC3)c3ccccc3-5)n2)cc1] | 196 |
| 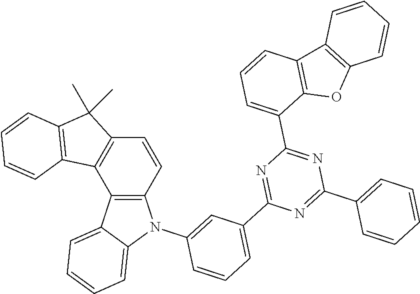 [SMILES: CC1(C)c2ccccc2-c2c1ccc1c2c2ccccc2n1-c1cccc(-c2nc(-c3ccccc3)nc(-c3cccc4c3oc3ccccc34)n2)c1] | 197 |
| 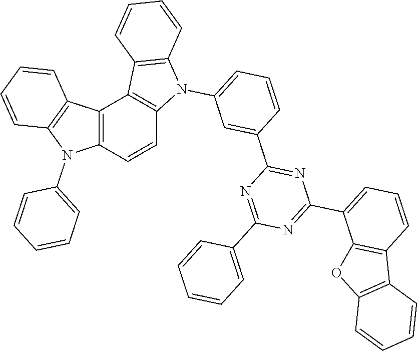 [SMILES: c1ccc(-c2nc(-c3cccc(-n4c5ccccc5c5c6c7ccccc7n(-c7ccccc7)c6ccc54)c3)nc(-c3cccc4c3oc3ccccc34)n2)cc1] | 198 |
| 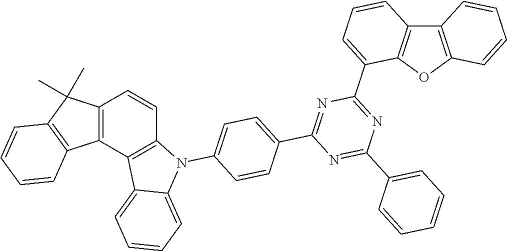 [SMILES: CC1(C)c2ccccc2-c2c1ccc1c2c2ccccc2n1-c1ccc(-c2nc(-c3ccccc3)nc(-c3cccc4c3oc3ccccc34)n2)cc1] | 199 |
| 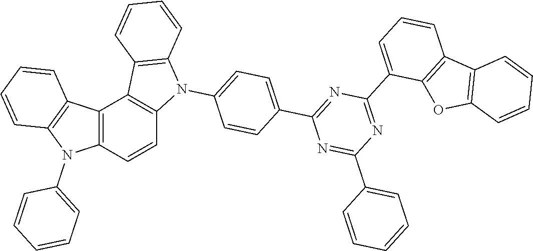 [SMILES: c1ccc(-c2nc(-c3ccc(-n4c5ccccc5c5c6c7ccccc7n(-c7ccccc7)c6ccc54)cc3)nc(-c3cccc4c3oc3ccccc34)n2)cc1] | 200 |
| 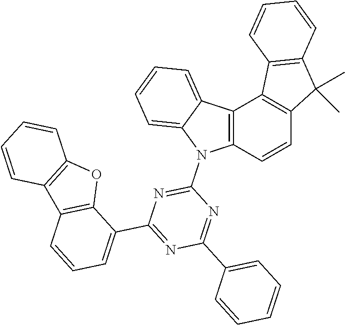 [SMILES: CC1(C)c2ccccc2-c2c1ccc1c2c2ccccc2n1-c1nc(-c2ccccc2)nc(-c2cccc3c2oc2ccccc23)n1] | 201 |
| 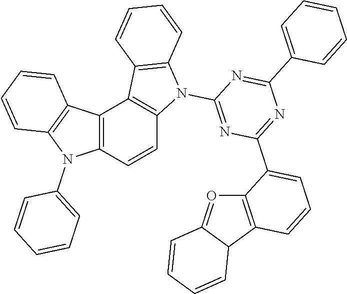 | 202 |
| 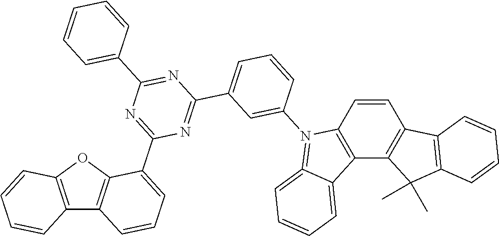 | 203 |
| 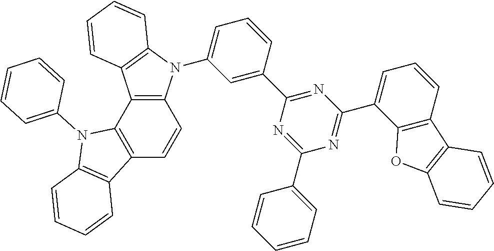 [SMILES: c1ccc(-c2nc(-c3cccc(-n4c5ccccc5c5c4ccc4c6ccccc6n(-c6ccccc6)c45)c3)nc(-c3cccc4c3oc3ccccc34)n2)cc1] | 204 |
| 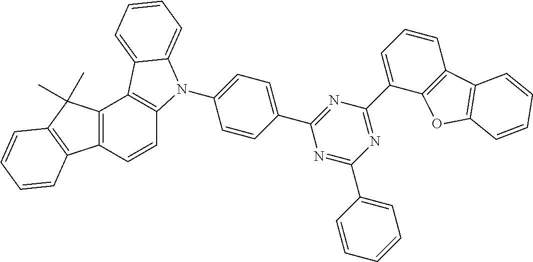 [SMILES: CC1(C)c2ccccc2-c2ccc3c(c21)c1ccccc1n3-c1ccc(-c2nc(-c3ccccc3)nc(-c3cccc4c3oc3ccccc34)n2)cc1] | 205 |
| 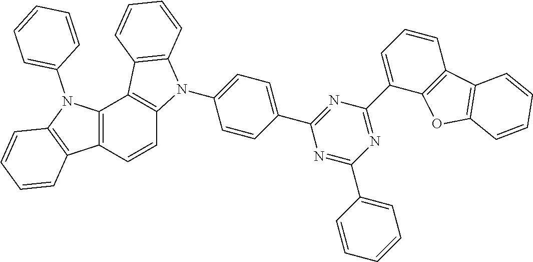 [SMILES: c1ccc(-c2nc(-c3ccc(-n4c5ccccc5c5c4ccc4c6ccccc6n(-c6ccccc6)c45)cc3)nc(-c3cccc4c3oc3ccccc34)n2)cc1] | 206 |
| 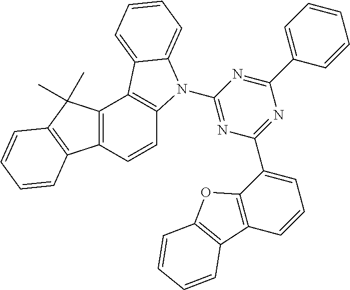 [SMILES: CC1(C)c2ccccc2-c2ccc3c(c21)c1ccccc1n3-c1nc(-c2ccccc2)nc(-c2cccc3c2oc2ccccc23)n1] | 207 |
| 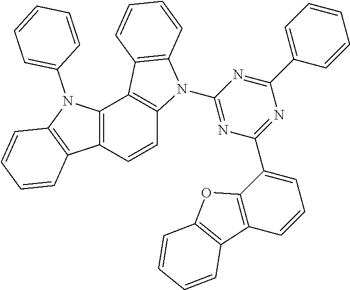 [SMILES: c1ccc(-c2nc(-c3cccc4c3oc3ccccc34)nc(-n3c4ccccc4c4c3ccc3c5ccccc5n(-c5ccccc5)c34)n2)cc1] | 208 |
| 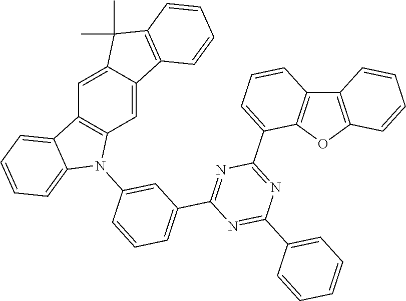 [SMILES: CC1(C)c2ccccc2-c2cc3c(cc21)c1ccccc1n3-c1cccc(-c2nc(-c3ccccc3)nc(-c3cccc4c3oc3ccccc34)n2)c1] | 209 |
| 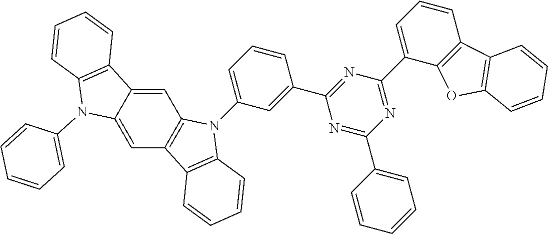 [SMILES: c1ccc(-c2nc(-c3cccc(-n4c5ccccc5c5cc6c(cc54)c4ccccc4n6-c4ccccc4)c3)nc(-c3cccc4c3oc3ccccc34)n2)cc1] | 210 |
| 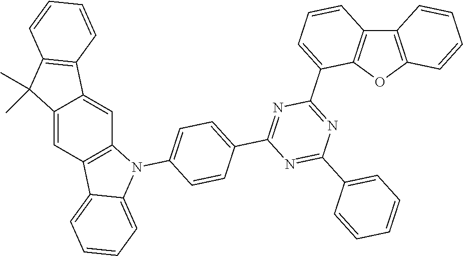 [SMILES: CC1(C)c2ccccc2-c2cc3c(cc21)c1ccccc1n3-c1ccc(-c2nc(-c3ccccc3)nc(-c3cccc4c3oc3ccccc34)n2)cc1] | 211 |
| 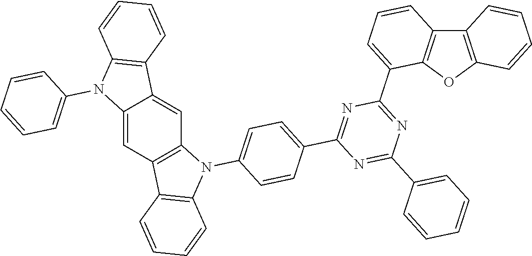 [SMILES: c1ccc(-c2nc(-c3ccc(-n4c5ccccc5c5cc6c(cc54)c4ccccc4n6-c4ccccc4)cc3)nc(-c3cccc4c3oc3ccccc34)n2)cc1] | 212 |
| 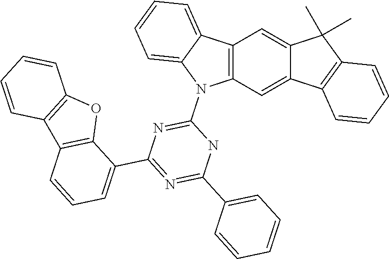 | 213 |
| 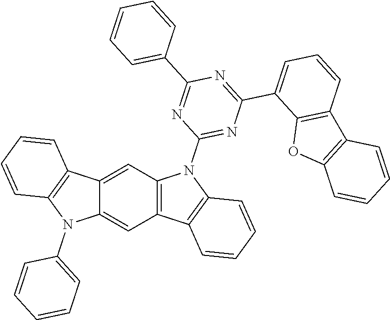 [SMILES: c1ccc(-c2nc(-c3cccc4c3oc3ccccc34)nc(-n3c4ccccc4c4cc5c(cc43)c3ccccc3n5-c3ccccc3)n2)cc1] | 214 |
| 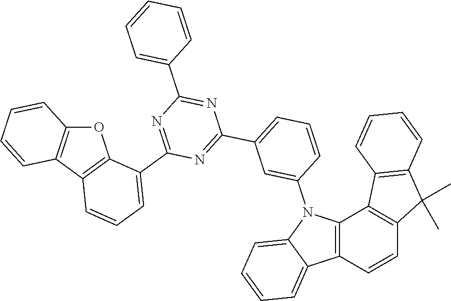 [SMILES: CC1(C)c2ccccc2-c2c1ccc1c3ccccc3n(-c3cccc(-c4nc(-c5ccccc5)nc(-c5cccc6c5oc5ccccc56)n4)c3)c21] | 215 |
| 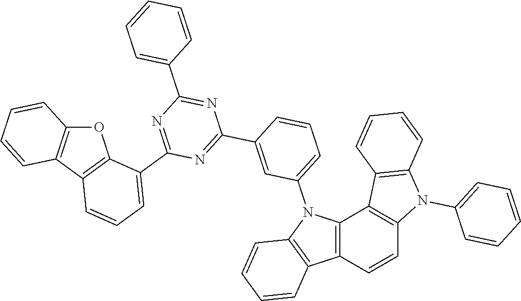 [SMILES: c1ccc(-c2nc(-c3cccc(-n4c5ccccc5c5ccc6c(c7ccccc7n6-c6ccccc6)c54)c3)nc(-c3cccc4c3oc3ccccc34)n2)cc1] | 216 |
| 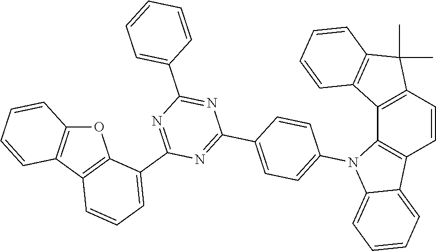 [SMILES: CC1(C)c2ccccc2-c2c1ccc1c3ccccc3n(-c3ccc(-c4nc(-c5ccccc5)nc(-c5cccc6c5oc5ccccc56)n4)cc3)c21] | 217 |
| 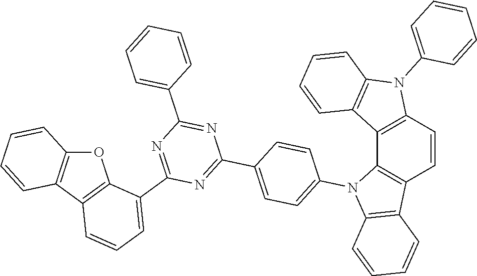 [SMILES: c1ccc(-c2nc(-c3ccc(-n4c5ccccc5c5ccc6c(c7ccccc7n6-c6ccccc6)c54)cc3)nc(-c3cccc4c3oc3ccccc34)n2)cc1] | 218 |
| 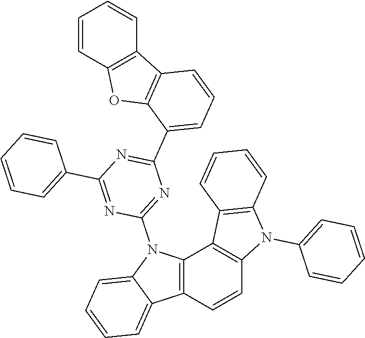 [SMILES: c1ccc(-c2nc(-c3cccc4c3oc3ccccc34)nc(-n3c4ccccc4c4ccc5c(c6ccccc6n5-c5ccccc5)c43)n2)cc1] | 219 |
| 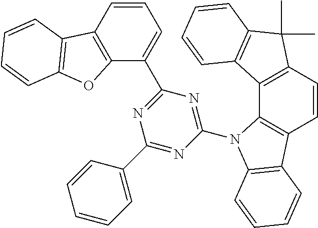 [SMILES: CC1(C)c2ccccc2-c2c1ccc1c3ccccc3n(-c3nc(-c4ccccc4)nc(-c4cccc5c4oc4ccccc45)n3)c21] | 220 |
| 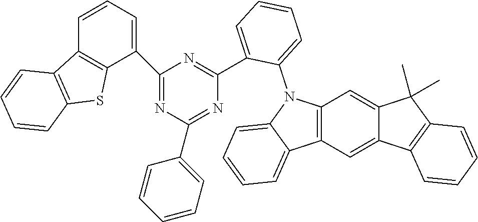 [SMILES: CC1(C)c2ccccc2-c2cc3c4ccccc4n(-c4ccccc4-c4nc(-c5ccccc5)nc(-c5cccc6c5sc5ccccc56)n4)c3cc21] | 221 |
| 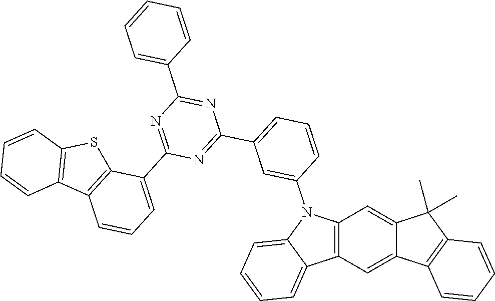 [SMILES: CC1(C)c2ccccc2-c2cc3c4ccccc4n(-c4cccc(-c5nc(-c6ccccc6)nc(-c6cccc7c6sc6ccccc67)n5)c4)c3cc21] | 222 |
| 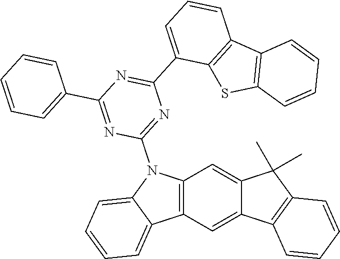 [SMILES: CC1(C)c2ccccc2-c2cc3c4ccccc4n(-c4nc(-c5ccccc5)nc(-c5cccc6c5sc5ccccc56)n4)c3cc21] | 223 |
| 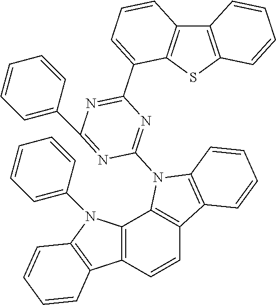 [SMILES: c1ccc(-c2nc(-c3cccc4c3sc3ccccc34)nc(-n3c4ccccc4c4ccc5c6ccccc6n(-c6ccccc6)c5c43)n2)cc1] | 224 |
| 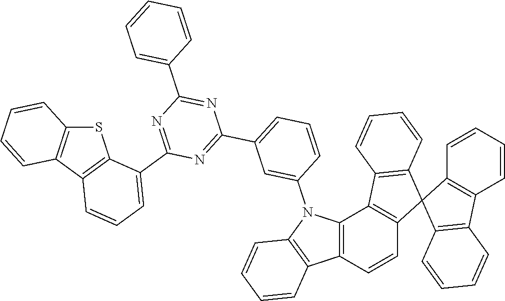 [SMILES: c1ccc(-c2nc(-c3cccc(-n4c5ccccc5c5ccc6c(c54)-c4ccccc4C64c5ccccc5-c5ccccc54)c3)nc(-c3cccc4c3sc3ccccc34)n2)cc1] | 225 |
| 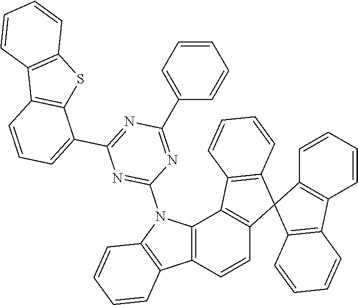 [SMILES: c1ccc(-c2nc(-c3cccc4c3sc3ccccc34)nc(-n3c4ccccc4c4ccc5c(c43)-c3ccccc3C53c4ccccc4-c4ccccc43)n2)cc1] | 226 |
| 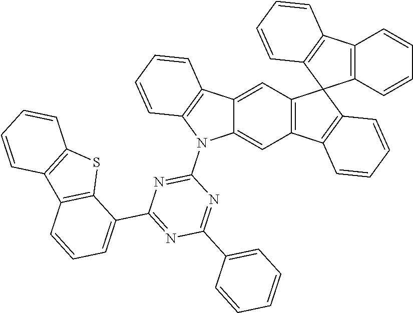 [SMILES: c1ccc(-c2nc(-c3cccc4c3sc3ccccc34)nc(-n3c4ccccc4c4cc5c(cc43)-c3ccccc3C53c4ccccc4-c4ccccc43)n2)cc1] | 227 |
| 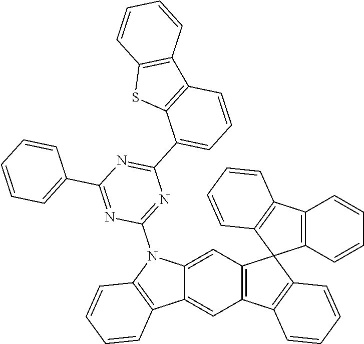 [SMILES: c1ccc(-c2nc(-c3cccc4c3sc3ccccc34)nc(-n3c4ccccc4c4cc5c(cc43)C3(c4ccccc4-c4ccccc43)c3ccccc3-5)n2)cc1] | 228 |
| 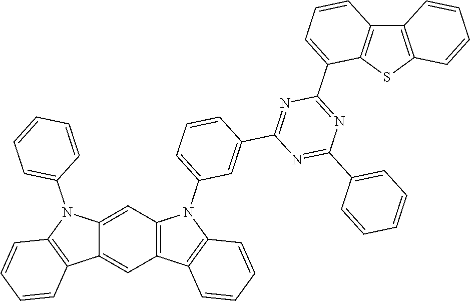 [SMILES: c1ccc(-c2nc(-c3cccc(-n4c5ccccc5c5cc6c7ccccc7n(-c7ccccc7)c6cc54)c3)nc(-c3cccc4c3sc3ccccc34)n2)cc1] | 229 |
| 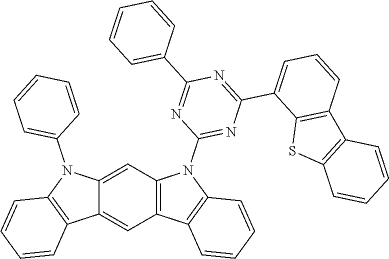 [SMILES: c1ccc(-c2nc(-c3cccc4c3sc3ccccc34)nc(-n3c4ccccc4c4cc5c6ccccc6n(-c6ccccc6)c5cc43)n2)cc1] | 230 |
| 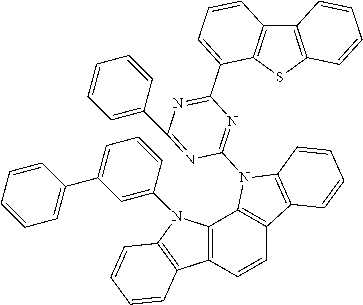 [SMILES: c1ccc(-c2cccc(-n3c4ccccc4c4ccc5c6ccccc6n(-c6nc(-c7ccccc7)nc(-c7cccc8c7sc7ccccc78)n6)c5c43)c2)cc1] | 231 |
| 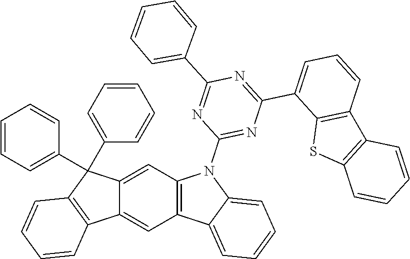 [SMILES: c1ccc(-c2nc(-c3cccc4c3sc3ccccc34)nc(-n3c4ccccc4c4cc5c(cc43)C(c3ccccc3)(c3ccccc3)c3ccccc3-5)n2)cc1] | 232 |
| 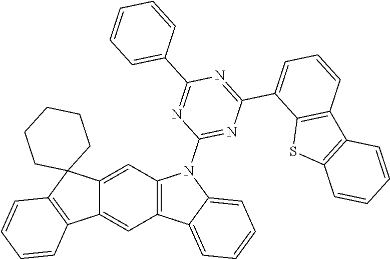 [SMILES: c1ccc(-c2nc(-c3cccc4c3sc3ccccc34)nc(-n3c4ccccc4c4cc5c(cc43)C3(CCCCC3)c3ccccc3-5)n2)cc1] | 233 |
| 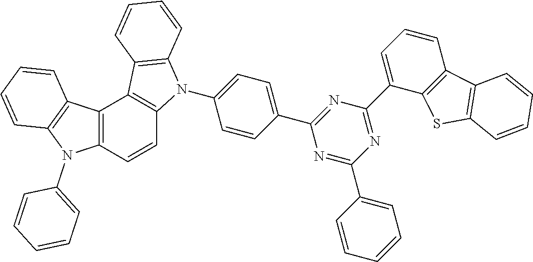 [SMILES: c1ccc(-c2nc(-c3ccc(-n4c5ccccc5c5c6c7ccccc7n(-c7ccccc7)c6ccc54)cc3)nc(-c3cccc4c3sc3ccccc34)n2)cc1] | 234 |
| 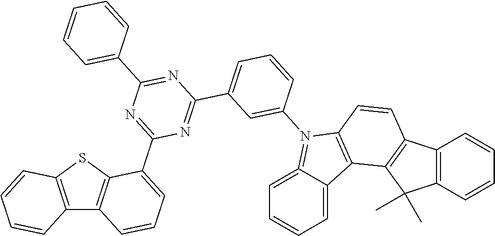 | 235 |
| 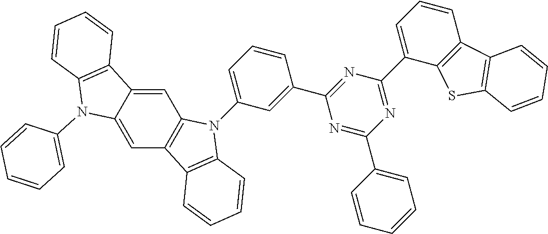 [SMILES: c1ccc(-c2nc(-c3cccc(-n4c5ccccc5c5cc6c(cc54)c4ccccc4n6-c4ccccc4)c3)nc(-c3cccc4c3sc3ccccc34)n2)cc1] | 236 |
| 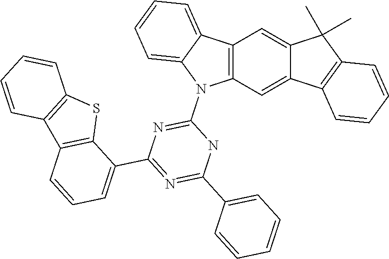 | 237 |
| 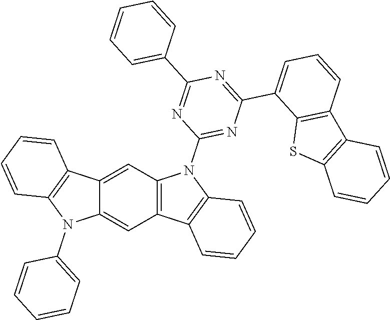 [SMILES: c1ccc(-c2nc(-c3cccc4c3sc3ccccc34)nc(-n3c4ccccc4c4cc5c(cc43)c3ccccc3n5-c3ccccc3)n2)cc1] | 238 |
| 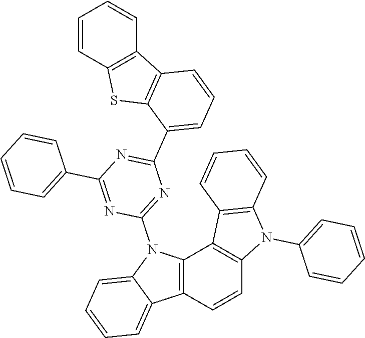 [SMILES: c1ccc(-c2nc(-c3cccc4c3sc3ccccc34)nc(-n3c4ccccc4c4ccc5c(c6ccccc6n5-c5ccccc5)c43)n2)cc1] | 239 |
| 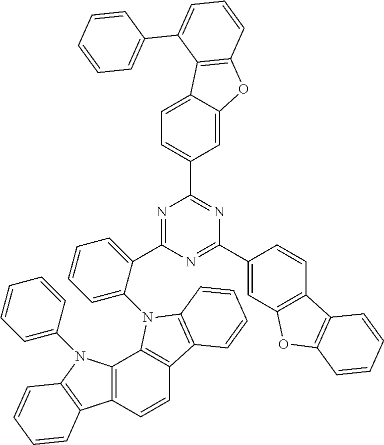 [SMILES: c1ccc(-c2cccc3oc4cc(-c5nc(-c6ccc7c(c6)oc6ccccc67)nc(-c6ccccc6-n6c7ccccc7c7ccc8c9ccccc9n(-c9ccccc9)c8c76)n5)ccc4c23)cc1] | 240 |
| 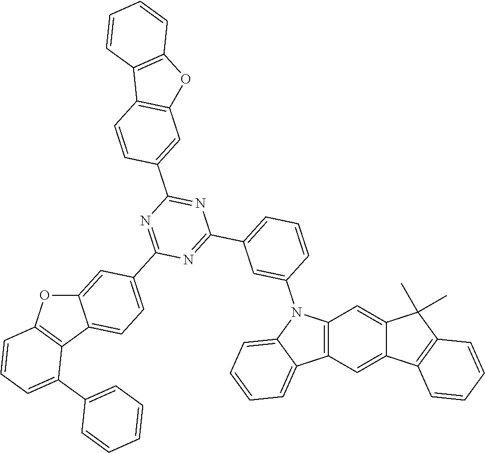 [SMILES: CC1(C)c2ccccc2-c2cc3c4ccccc4n(-c4cccc(-c5nc(-c6ccc7c(c6)oc6ccccc67)nc(-c6ccc7c(c6)oc6cccc(-c8ccccc8)c67)n5)c4)c3cc21] | 241 |
| 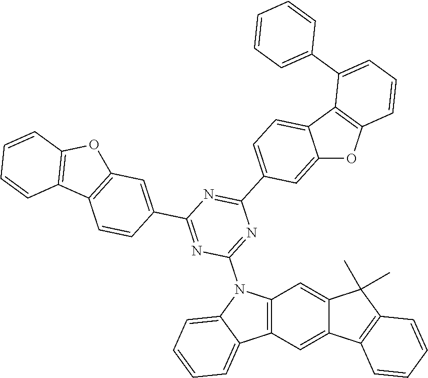 [SMILES: CC1(C)c2ccccc2-c2cc3c4ccccc4n(-c4nc(-c5ccc6c(c5)oc5ccccc56)nc(-c5ccc6c(c5)oc5cccc(-c7ccccc7)c56)n4)c3cc21] | 242 |
| 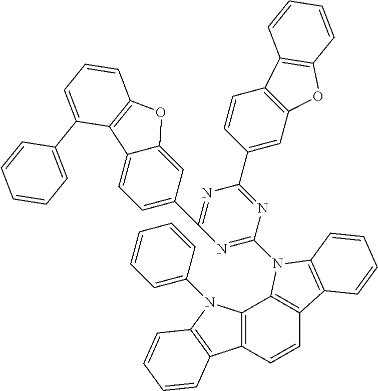 [SMILES: c1ccc(-c2cccc3oc4cc(-c5nc(-c6ccc7c(c6)oc6ccccc67)nc(-n6c7ccccc7c7ccc8c9ccccc9n(-c9ccccc9)c8c76)n5)ccc4c23)cc1] | 243 |
| 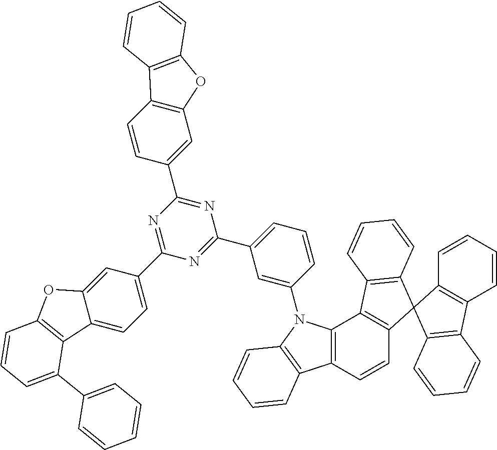 [SMILES: c1ccc(-c2cccc3oc4cc(-c5nc(-c6cccc(-n7c8ccccc8c8ccc9c(c87)-c7ccccc7C97c8ccccc8-c8ccccc87)c6)nc(-c6ccc7c(c6)oc6ccccc67)n5)ccc4c23)cc1] | 244 |
| 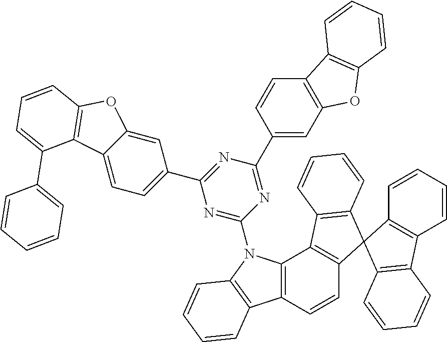 [SMILES: c1ccc(-c2cccc3oc4cc(-c5nc(-c6ccc7c(c6)oc6ccccc67)nc(-n6c7ccccc7c7ccc8c(c76)-c6ccccc6C86c7ccccc7-c7ccccc76)n5)ccc4c23)cc1] | 245 |
| 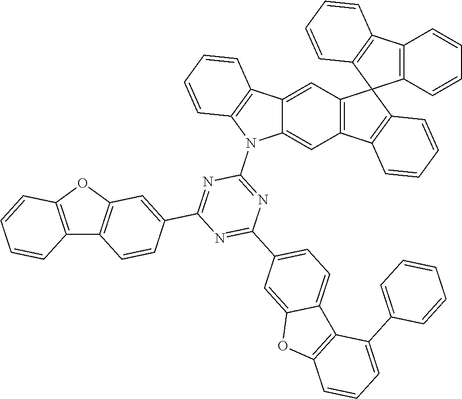 [SMILES: c1ccc(-c2cccc3oc4cc(-c5nc(-c6ccc7c(c6)oc6ccccc67)nc(-n6c7ccccc7c7cc8c(cc76)-c6ccccc6C86c7ccccc7-c7ccccc76)n5)ccc4c23)cc1] | 246 |
| 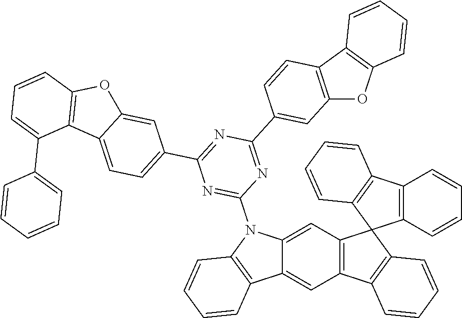 [SMILES: c1ccc(-c2cccc3oc4cc(-c5nc(-c6ccc7c(c6)oc6ccccc67)nc(-n6c7ccccc7c7cc8c(cc76)C6(c7ccccc7-c7ccccc76)c6ccccc6-8)n5)ccc4c23)cc1] | 247 |
| 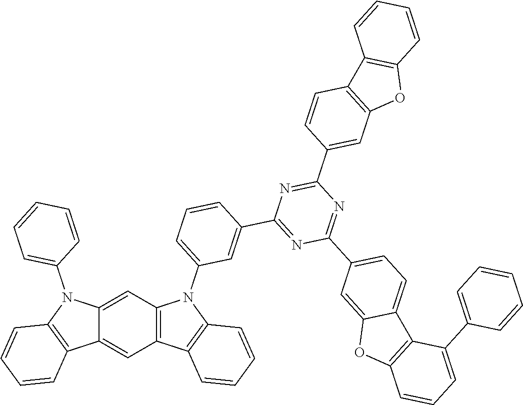 [SMILES: c1ccc(-c2cccc3oc4cc(-c5nc(-c6cccc(-n7c8ccccc8c8cc9c%10ccccc%10n(-c%10ccccc%10)c9cc87)c6)nc(-c6ccc7c(c6)oc6ccccc67)n5)ccc4c23)cc1] | 248 |
| 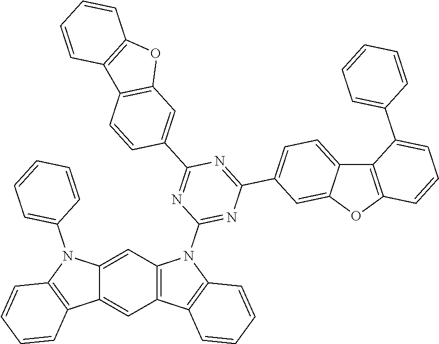 [SMILES: c1ccc(-c2cccc3oc4cc(-c5nc(-c6ccc7c(c6)oc6ccccc67)nc(-n6c7ccccc7c7cc8c9ccccc9n(-c9ccccc9)c8cc76)n5)ccc4c23)cc1] | 249 |
| 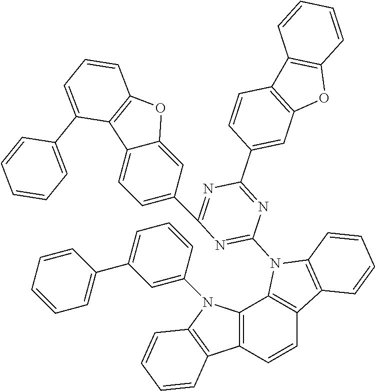 [SMILES: c1ccc(-c2cccc(-n3c4ccccc4c4ccc5c6ccccc6n(-c6nc(-c7ccc8c(c7)oc7ccccc78)nc(-c7ccc8c(c7)oc7cccc(-c9ccccc9)c78)n6)c5c43)c2)cc1] | 250 |
| 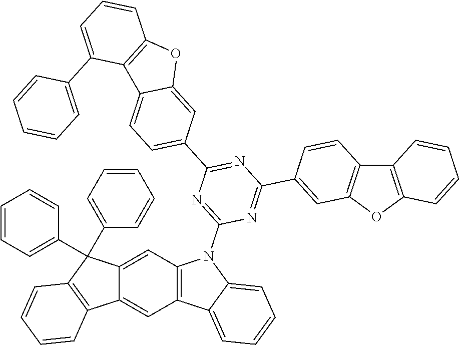 [SMILES: c1ccc(-c2cccc3oc4cc(-c5nc(-c6ccc7c(c6)oc6ccccc67)nc(-n6c7ccccc7c7cc8c(cc76)C(c6ccccc6)(c6ccccc6)c6ccccc6-8)n5)ccc4c23)cc1] | 251 |
| 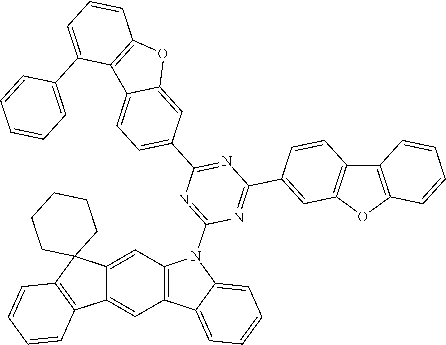 [SMILES: c1ccc(-c2cccc3oc4cc(-c5nc(-c6ccc7c(c6)oc6ccccc67)nc(-n6c7ccccc7c7cc8c(cc76)C6(CCCCC6)c6ccccc6-8)n5)ccc4c23)cc1] | 252 |
| 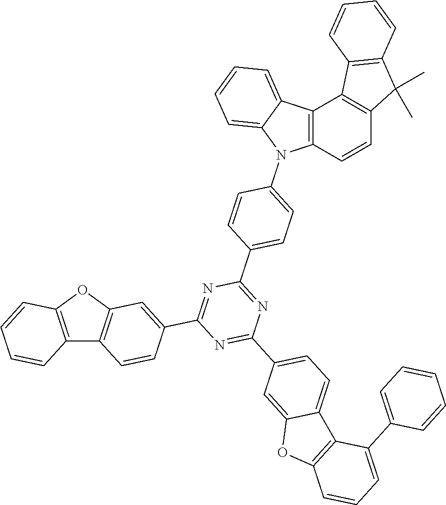 [SMILES: CC1(C)c2ccccc2-c2c1ccc1c2c2ccccc2n1-c1ccc(-c2nc(-c3ccc4c(c3)oc3ccccc34)nc(-c3ccc4c(c3)oc3cccc(-c5ccccc5)c34)n2)cc1] | 253 |
| 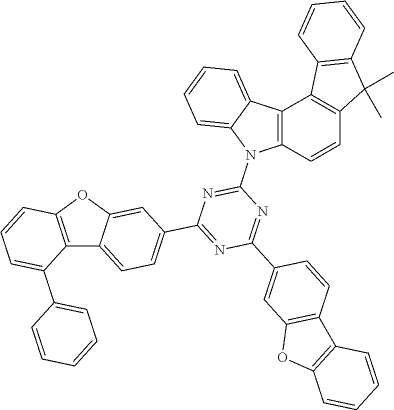 [SMILES: CC1(C)c2ccccc2-c2c1ccc1c2c2ccccc2n1-c1nc(-c2ccc3c(c2)oc2ccccc23)nc(-c2ccc3c(c2)oc2cccc(-c4ccccc4)c23)n1] | 254 |
| 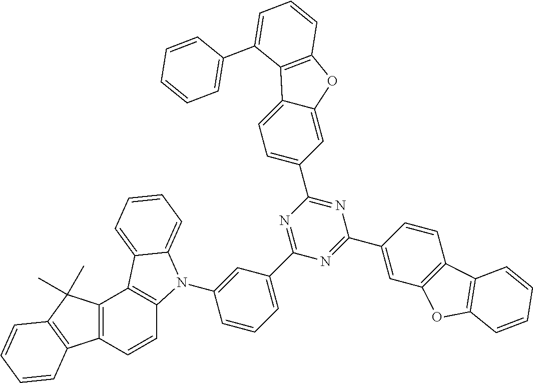 [SMILES: CC1(C)c2ccccc2-c2ccc3c(c21)c1ccccc1n3-c1cccc(-c2nc(-c3ccc4c(c3)oc3ccccc34)nc(-c3ccc4c(c3)oc3cccc(-c5ccccc5)c34)n2)c1] | 255 |
| 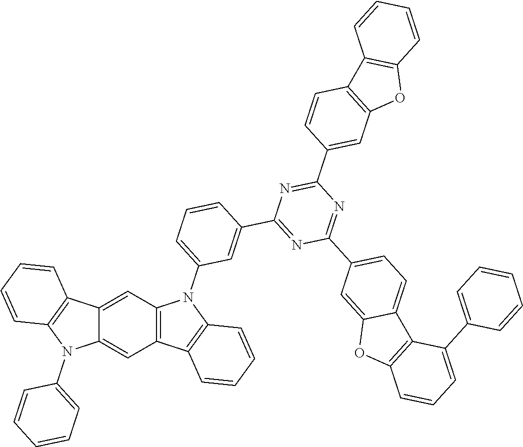 [SMILES: c1ccc(-c2cccc3oc4cc(-c5nc(-c6cccc(-n7c8ccccc8c8cc9c(cc87)c7ccccc7n9-c7ccccc7)c6)nc(-c6ccc7c(c6)oc6ccccc67)n5)ccc4c23)cc1] | 256 |
| 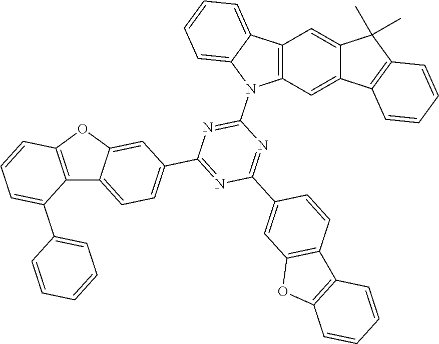 [SMILES: CC1(C)c2ccccc2-c2cc3c(cc21)c1ccccc1n3-c1nc(-c2ccc3c(c2)oc2ccccc23)nc(-c2ccc3c(c2)oc2cccc(-c4ccccc4)c23)n1] | 257 |
| 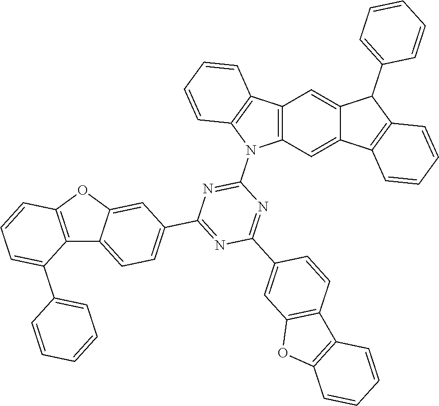 [SMILES: c1ccc(-c2cccc3oc4cc(-c5nc(-c6ccc7c(c6)oc6ccccc67)nc(-n6c7ccccc7c7cc8c(cc76)-c6ccccc6C8c6ccccc6)n5)ccc4c23)cc1] | 258 |
| 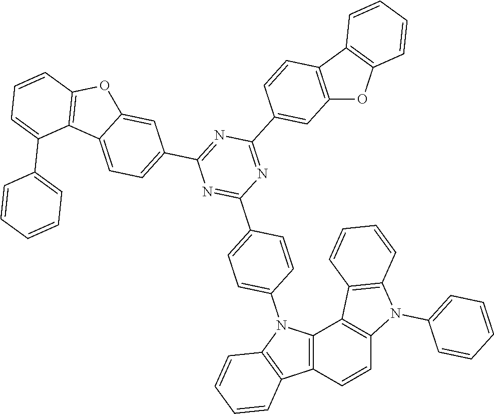 [SMILES: c1ccc(-c2cccc3oc4cc(-c5nc(-c6ccc(-n7c8ccccc8c8ccc9c(c%10ccccc%10n9-c9ccccc9)c87)cc6)nc(-c6ccc7c(c6)oc6ccccc67)n5)ccc4c23)cc1] | 259 |
| 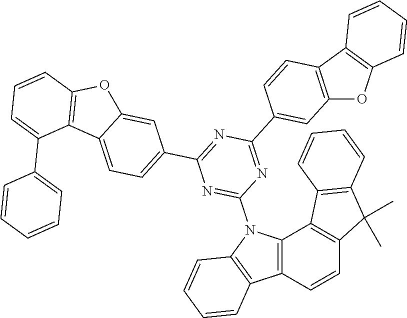 [SMILES: CC1(C)c2ccccc2-c2c1ccc1c3ccccc3n(-c3nc(-c4ccc5c(c4)oc4ccccc45)nc(-c4ccc5c(c4)oc4cccc(-c6ccccc6)c45)n3)c21] | 260 |
| 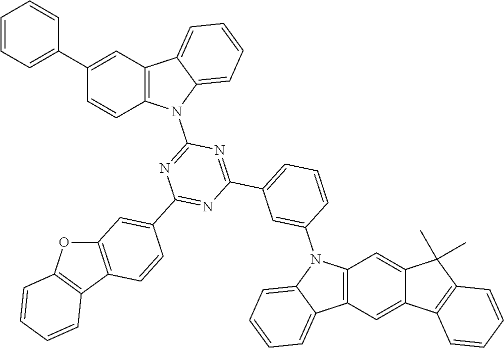 [SMILES: CC1(C)c2ccccc2-c2cc3c4ccccc4n(-c4cccc(-c5nc(-c6ccc7c(c6)oc6ccccc67)nc(-n6c7ccccc7c7cc(-c8ccccc8)ccc76)n5)c4)c3cc21] | 261 |
| 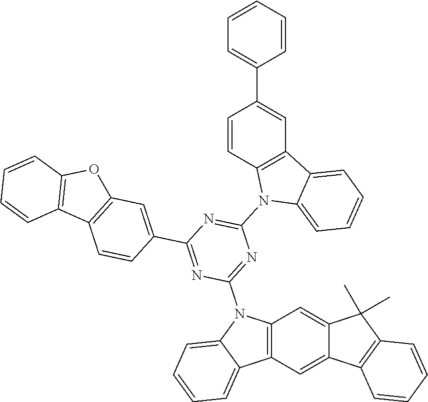 [SMILES: CC1(C)c2ccccc2-c2cc3c4ccccc4n(-c4nc(-c5ccc6c(c5)oc5ccccc56)nc(-n5c6ccccc6c6cc(-c7ccccc7)ccc65)n4)c3cc21] | 262 |
| 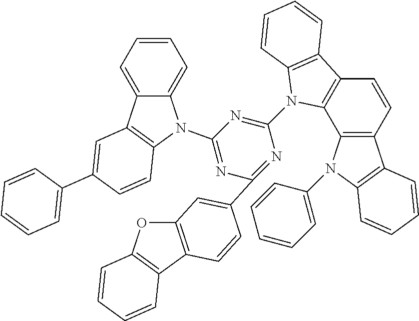 [SMILES: c1ccc(-c2ccc3c(c2)c2ccccc2n3-c2nc(-c3ccc4c(c3)oc3ccccc34)nc(-n3c4ccccc4c4ccc5c6ccccc6n(-c6ccccc6)c5c43)n2)cc1] | 263 |
| 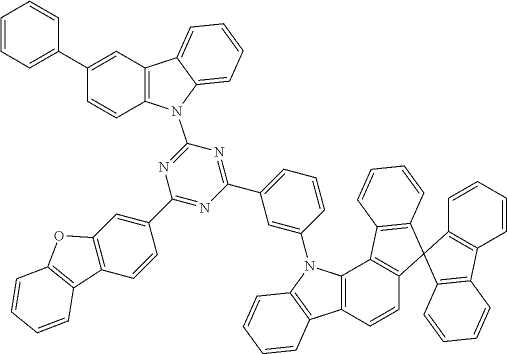 [SMILES: c1ccc(-c2ccc3c(c2)c2ccccc2n3-c2nc(-c3cccc(-n4c5ccccc5c5ccc6c(c54)-c4ccccc4C64c5ccccc5-c5ccccc54)c3)nc(-c3ccc4c(c3)oc3ccccc34)n2)cc1] | 264 |
| 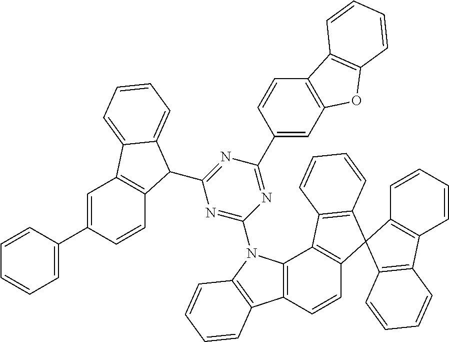 [SMILES: c1ccc(-c2ccc3c(c2)-c2ccccc2C3c2nc(-c3ccc4c(c3)oc3ccccc34)nc(-n3c4ccccc4c4ccc5c(c43)-c3ccccc3C53c4ccccc4-c4ccccc43)n2)cc1] | 265 |
| 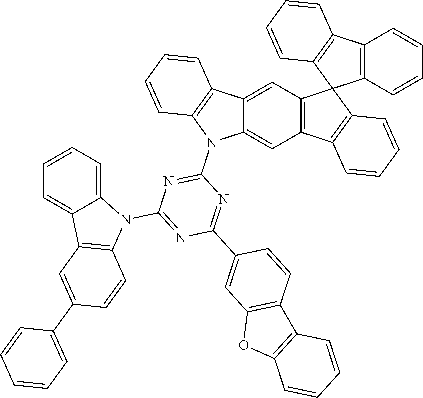 [SMILES: c1ccc(-c2ccc3c(c2)c2ccccc2n3-c2nc(-c3ccc4c(c3)oc3ccccc34)nc(-n3c4ccccc4c4cc5c(cc43)-c3ccccc3C53c4ccccc4-c4ccccc43)n2)cc1] | 266 |
| 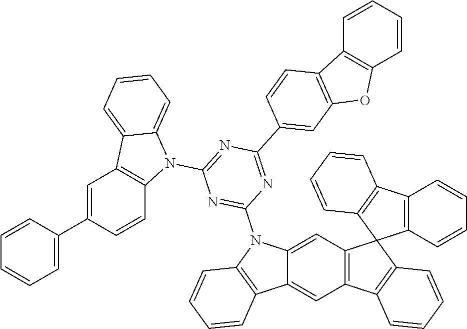 [SMILES: c1ccc(-c2ccc3c(c2)c2ccccc2n3-c2nc(-c3ccc4c(c3)oc3ccccc34)nc(-n3c4ccccc4c4cc5c(cc43)C3(c4ccccc4-c4ccccc43)c3ccccc3-5)n2)cc1] | 267 |
| 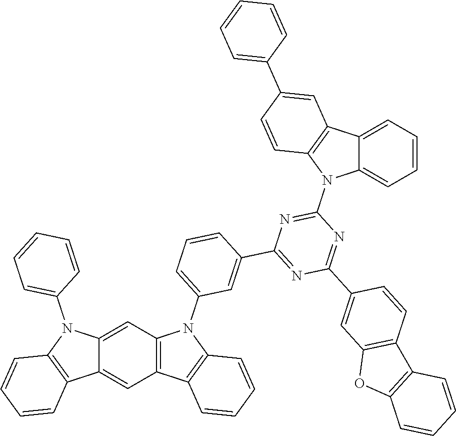 [SMILES: c1ccc(-c2ccc3c(c2)c2ccccc2n3-c2nc(-c3cccc(-n4c5ccccc5c5cc6c7ccccc7n(-c7ccccc7)c6cc54)c3)nc(-c3ccc4c(c3)oc3ccccc34)n2)cc1] | 268 |
| 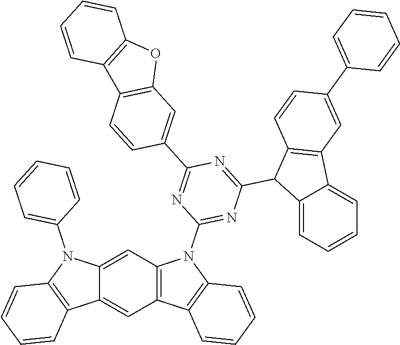 [SMILES: c1ccc(-c2ccc3c(c2)-c2ccccc2C3c2nc(-c3ccc4c(c3)oc3ccccc34)nc(-n3c4ccccc4c4cc5c6ccccc6n(-c6ccccc6)c5cc43)n2)cc1] | 269 |
| 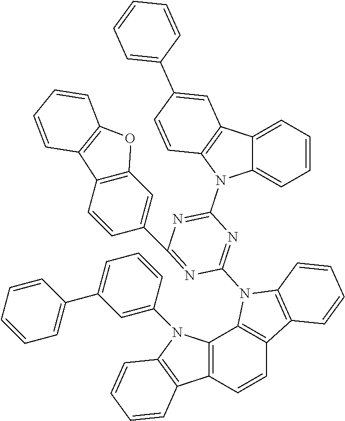 [SMILES: c1ccc(-c2cccc(-n3c4ccccc4c4ccc5c6ccccc6n(-c6nc(-c7ccc8c(c7)oc7ccccc78)nc(-n7c8ccccc8c8cc(-c9ccccc9)ccc87)n6)c5c43)c2)cc1] | 270 |
| 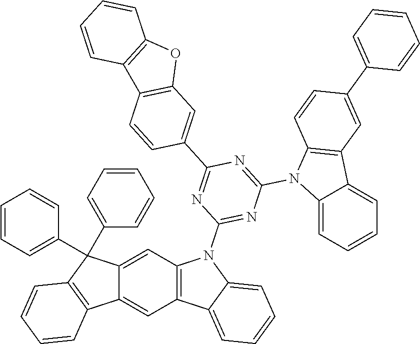 [SMILES: c1ccc(-c2ccc3c(c2)c2ccccc2n3-c2nc(-c3ccc4c(c3)oc3ccccc34)nc(-n3c4ccccc4c4cc5c(cc43)C(c3ccccc3)(c3ccccc3)c3ccccc3-5)n2)cc1] | 271 |
| 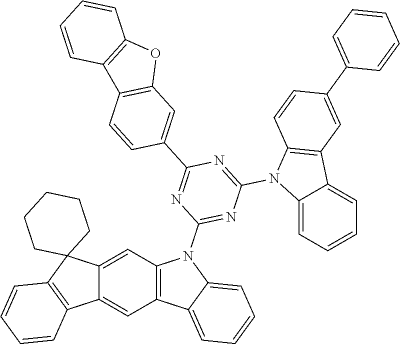 [SMILES: c1ccc(-c2ccc3c(c2)c2ccccc2n3-c2nc(-c3ccc4c(c3)oc3ccccc34)nc(-n3c4ccccc4c4cc5c(cc43)C3(CCCCC3)c3ccccc3-5)n2)cc1] | 272 |
| 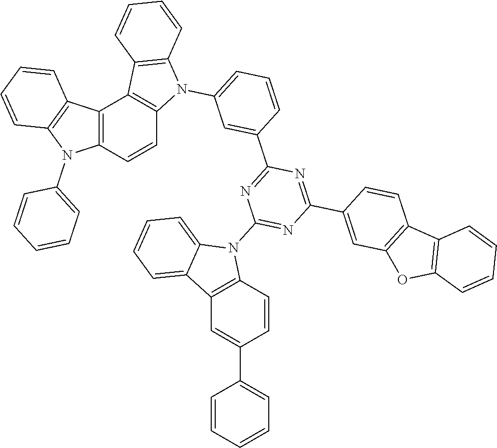 [SMILES: c1ccc(-c2ccc3c(c2)c2ccccc2n3-c2nc(-c3cccc(-n4c5ccccc5c5c6c7ccccc7n(-c7ccccc7)c6ccc54)c3)nc(-c3ccc4c(c3)oc3ccccc34)n2)cc1] | 273 |
| 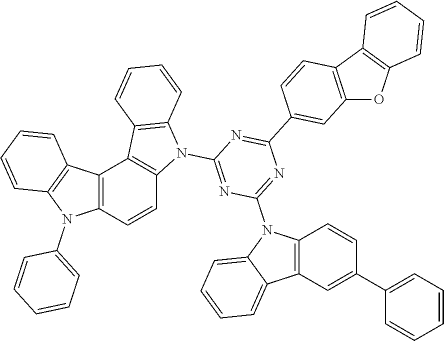 [SMILES: c1ccc(-c2ccc3c(c2)c2ccccc2n3-c2nc(-c3ccc4c(c3)oc3ccccc34)nc(-n3c4ccccc4c4c5c6ccccc6n(-c6ccccc6)c5ccc43)n2)cc1] | 274 |
| 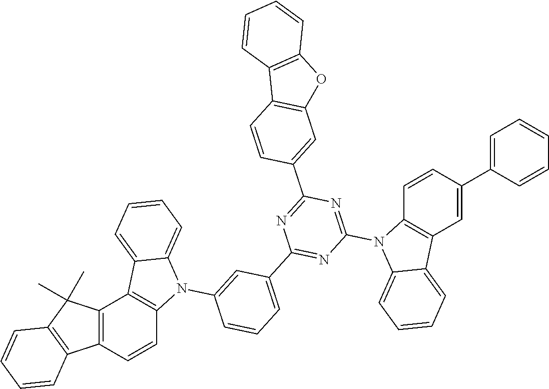 [SMILES: CC1(C)c2ccccc2-c2ccc3c(c21)c1ccccc1n3-c1cccc(-c2nc(-c3ccc4c(c3)oc3ccccc34)nc(-n3c4ccccc4c4cc(-c5ccccc5)ccc43)n2)c1] | 275 |
| 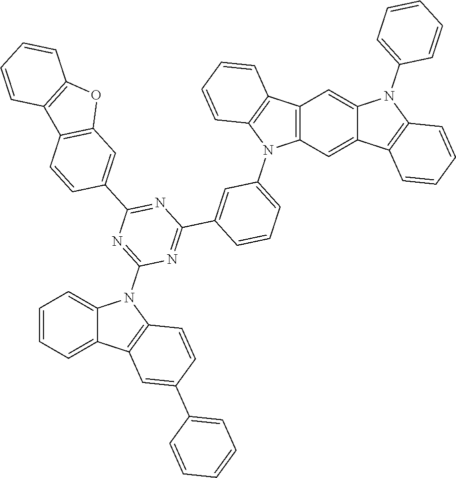 [SMILES: c1ccc(-c2ccc3c(c2)c2ccccc2n3-c2nc(-c3cccc(-n4c5ccccc5c5cc6c(cc54)c4ccccc4n6-c4ccccc4)c3)nc(-c3ccc4c(c3)oc3ccccc34)n2)cc1] | 276 |
| 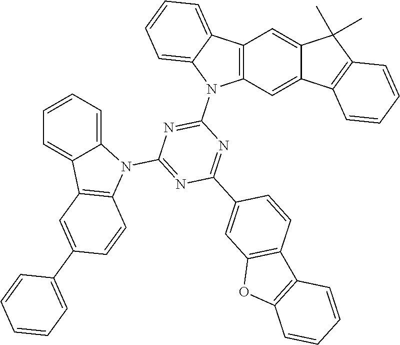 [SMILES: CC1(C)c2ccccc2-c2cc3c(cc21)c1ccccc1n3-c1nc(-c2ccc3c(c2)oc2ccccc23)nc(-n2c3ccccc3c3cc(-c4ccccc4)ccc32)n1] | 277 |
| 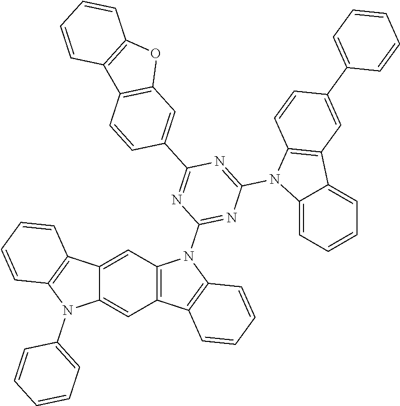 [SMILES: c1ccc(-c2ccc3c(c2)c2ccccc2n3-c2nc(-c3ccc4c(c3)oc3ccccc34)nc(-n3c4ccccc4c4cc5c(cc43)c3ccccc3n5-c3ccccc3)n2)cc1] | 278 |
| 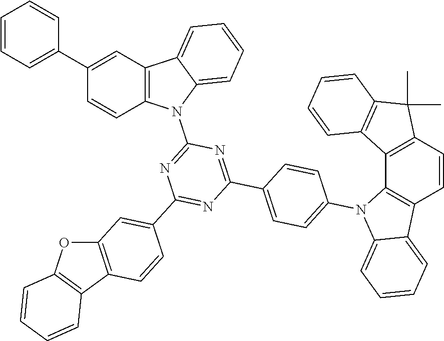 [SMILES: CC1(C)c2ccccc2-c2c1ccc1c3ccccc3n(-c3ccc(-c4nc(-c5ccc6c(c5)oc5ccccc56)nc(-n5c6ccccc6c6cc(-c7ccccc7)ccc65)n4)cc3)c21] | 279 |
| 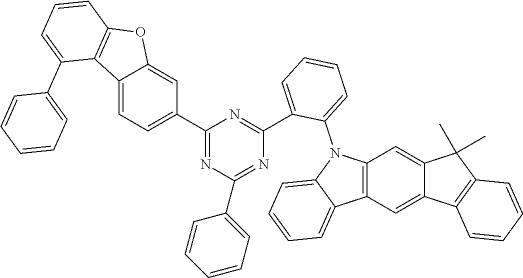 [SMILES: CC1(C)c2ccccc2-c2cc3c4ccccc4n(-c4ccccc4-c4nc(-c5ccccc5)nc(-c5ccc6c(c5)oc5cccc(-c7ccccc7)c56)n4)c3cc21] | 280 |
| 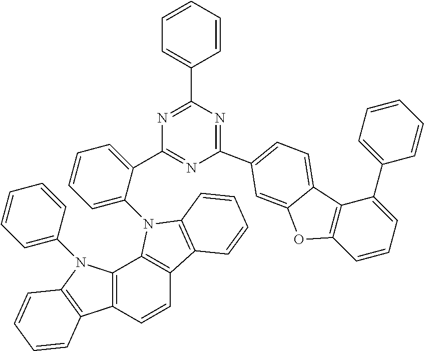 [SMILES: c1ccc(-c2nc(-c3ccc4c(c3)oc3cccc(-c5ccccc5)c34)nc(-c3ccccc3-n3c4ccccc4c4ccc5c6ccccc6n(-c6ccccc6)c5c43)n2)cc1] | 281 |
| 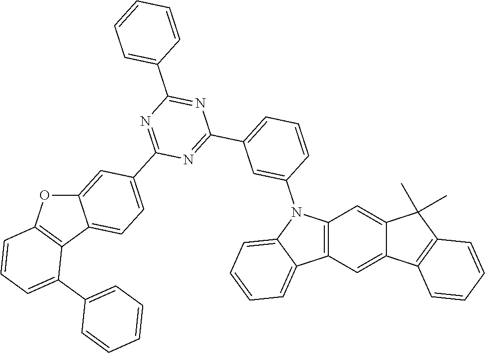 [SMILES: CC1(C)c2ccccc2-c2cc3c4ccccc4n(-c4cccc(-c5nc(-c6ccccc6)nc(-c6ccc7c(c6)oc6cccc(-c8ccccc8)c67)n5)c4)c3cc21] | 282 |
| 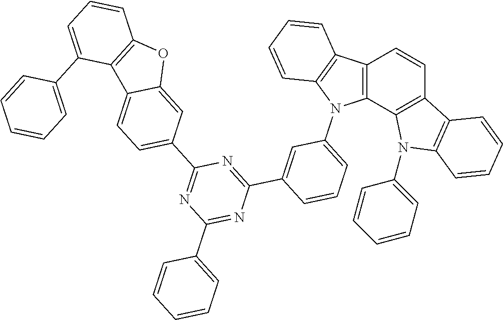 [SMILES: c1ccc(-c2nc(-c3cccc(-n4c5ccccc5c5ccc6c7ccccc7n(-c7ccccc7)c6c54)c3)nc(-c3ccc4c(c3)oc3cccc(-c5ccccc5)c34)n2)cc1] | 283 |
| 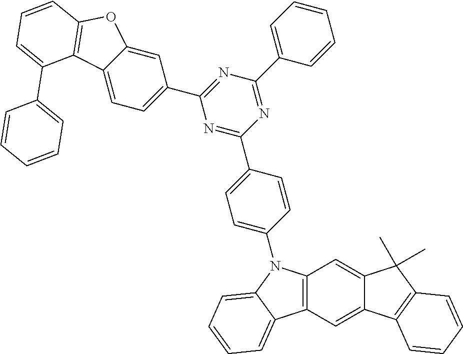 [SMILES: CC1(C)c2ccccc2-c2cc3c4ccccc4n(-c4ccc(-c5nc(-c6ccccc6)nc(-c6ccc7c(c6)oc6cccc(-c8ccccc8)c67)n5)cc4)c3cc21] | 284 |
| 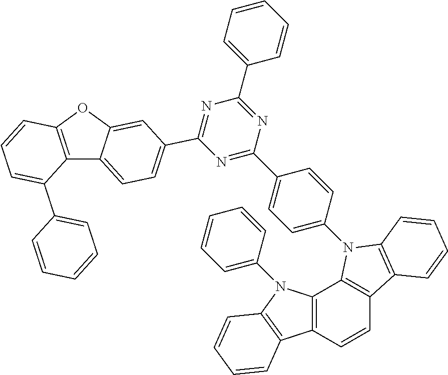 [SMILES: c1ccc(-c2nc(-c3ccc(-n4c5ccccc5c5ccc6c7ccccc7n(-c7ccccc7)c6c54)cc3)nc(-c3ccc4c(c3)oc3cccc(-c5ccccc5)c34)n2)cc1] | 285 |
| 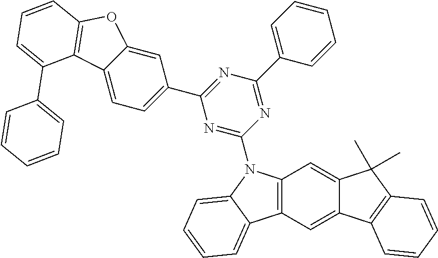 [SMILES: CC1(C)c2ccccc2-c2cc3c4ccccc4n(-c4nc(-c5ccccc5)nc(-c5ccc6c(c5)oc5cccc(-c7ccccc7)c56)n4)c3cc21] | 286 |
| 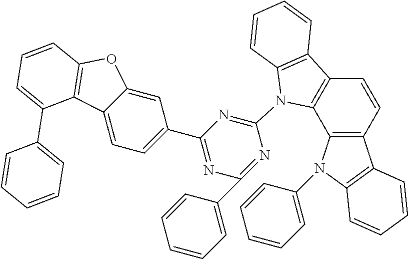 [SMILES: c1ccc(-c2nc(-c3ccc4c(c3)oc3cccc(-c5ccccc5)c34)nc(-n3c4ccccc4c4ccc5c6ccccc6n(-c6ccccc6)c5c43)n2)cc1] | 287 |
| 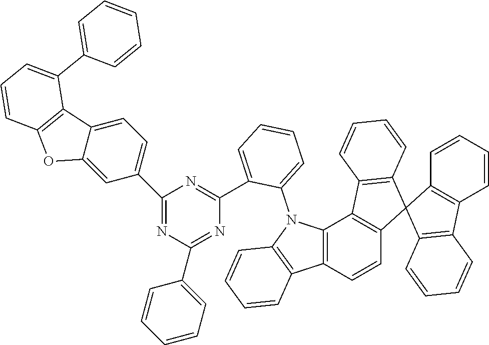 [SMILES: c1ccc(-c2nc(-c3ccc4c(c3)oc3cccc(-c5ccccc5)c34)nc(-c3ccccc3-n3c4ccccc4c4ccc5c(c43)-c3ccccc3C53c4ccccc4-c4ccccc43)n2)cc1] | 288 |
| 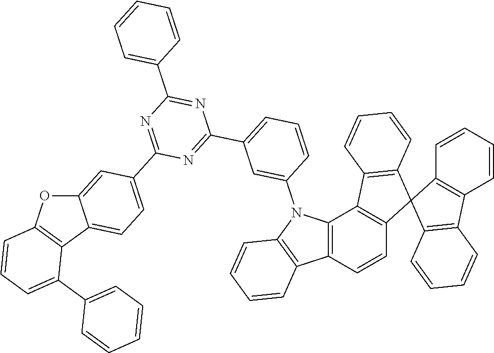 [SMILES: c1ccc(-c2nc(-c3cccc(-n4c5ccccc5c5ccc6c(c54)-c4ccccc4C64c5ccccc5-c5ccccc54)c3)nc(-c3ccc4c(c3)oc3cccc(-c5ccccc5)c34)n2)cc1] | 289 |
| 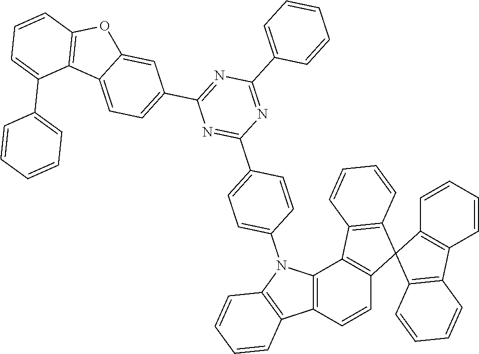 [SMILES: c1ccc(-c2nc(-c3ccc(-n4c5ccccc5c5ccc6c(c54)-c4ccccc4C64c5ccccc5-c5ccccc54)cc3)nc(-c3ccc4c(c3)oc3cccc(-c5ccccc5)c34)n2)cc1] | 290 |
| 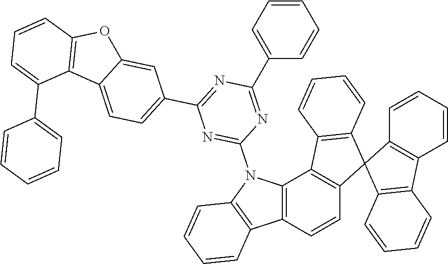 [SMILES: c1ccc(-c2nc(-c3ccc4c(c3)oc3cccc(-c5ccccc5)c34)nc(-n3c4ccccc4c4ccc5c(c43)-c3ccccc3C53c4ccccc4-c4ccccc43)n2)cc1] | 291 |
| 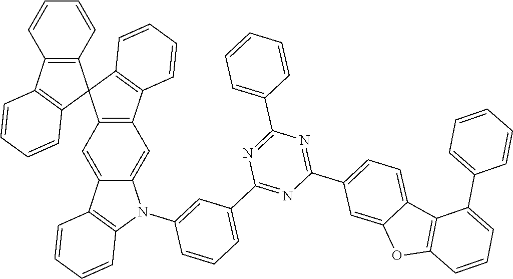 [SMILES: c1ccc(-c2nc(-c3cccc(-n4c5ccccc5c5cc6c(cc54)-c4ccccc4C64c5ccccc5-c5ccccc54)c3)nc(-c3ccc4c(c3)oc3cccc(-c5ccccc5)c34)n2)cc1] | 292 |
| 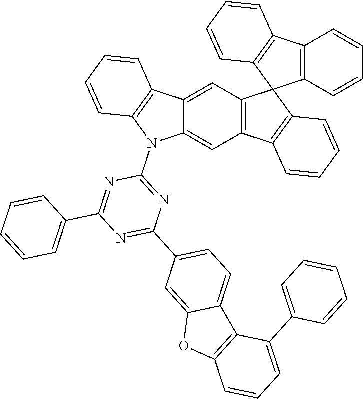 [SMILES: c1ccc(-c2nc(-c3ccc4c(c3)oc3cccc(-c5ccccc5)c34)nc(-n3c4ccccc4c4cc5c(cc43)-c3ccccc3C53c4ccccc4-c4ccccc43)n2)cc1] | 293 |
| 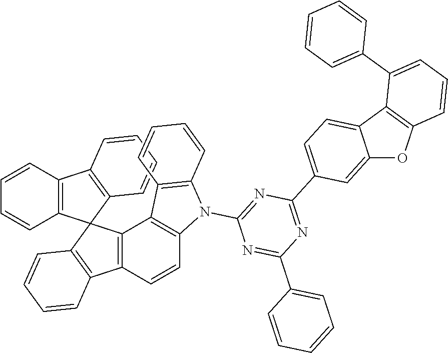 [SMILES: c1ccc(-c2nc(-c3ccc4c(c3)oc3cccc(-c5ccccc5)c34)nc(-n3c4ccccc4c4c5c(ccc43)-c3ccccc3C53c4ccccc4-c4ccccc43)n2)cc1] | 294 |
| 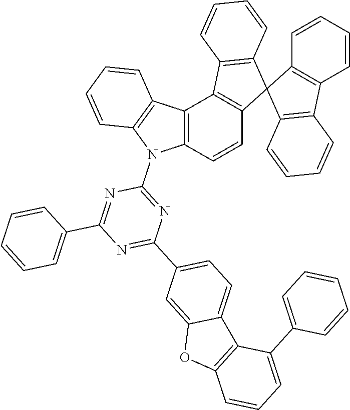 [SMILES: c1ccc(-c2nc(-c3ccc4c(c3)oc3cccc(-c5ccccc5)c34)nc(-n3c4ccccc4c4c5c(ccc43)C3(c4ccccc4-c4ccccc43)c3ccccc3-5)n2)cc1] | 295 |
| 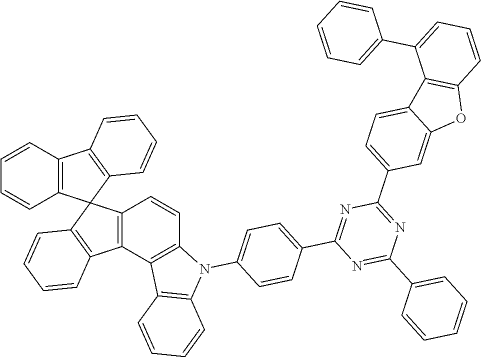 [SMILES: c1ccc(-c2nc(-c3ccc(-n4c5ccccc5c5c6c(ccc54)C4(c5ccccc5-c5ccccc54)c4ccccc4-6)cc3)nc(-c3ccc4c(c3)oc3cccc(-c5ccccc5)c34)n2)cc1] | 296 |
| 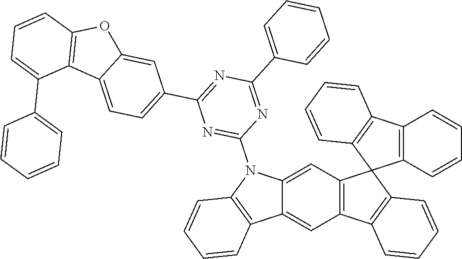 [SMILES: c1ccc(-c2nc(-c3ccc4c(c3)oc3cccc(-c5ccccc5)c34)nc(-n3c4ccccc4c4cc5c(cc43)C3(c4ccccc4-c4ccccc43)c3ccccc3-5)n2)cc1] | 297 |
| 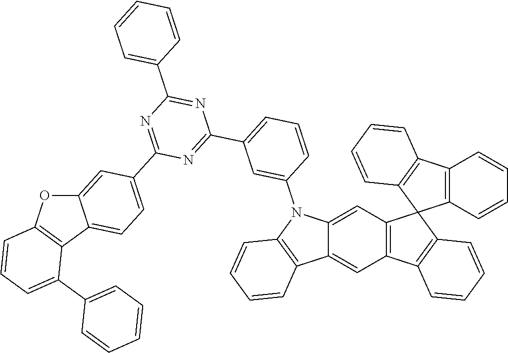 [SMILES: c1ccc(-c2nc(-c3cccc(-n4c5ccccc5c5cc6c(cc54)C4(c5ccccc5-c5ccccc54)c4ccccc4-6)c3)nc(-c3ccc4c(c3)oc3cccc(-c5ccccc5)c34)n2)cc1] | 298 |
| 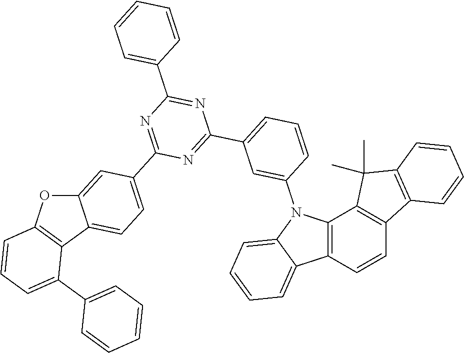 [SMILES: CC1(C)c2ccccc2-c2ccc3c4ccccc4n(-c4cccc(-c5nc(-c6ccccc6)nc(-c6ccc7c(c6)oc6cccc(-c8ccccc8)c67)n5)c4)c3c21] | 299 |
| 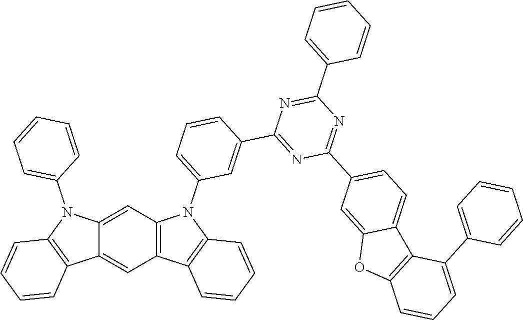 [SMILES: c1ccc(-c2nc(-c3cccc(-n4c5ccccc5c5cc6c7ccccc7n(-c7ccccc7)c6cc54)c3)nc(-c3ccc4c(c3)oc3cccc(-c5ccccc5)c34)n2)cc1] | 300 |
| 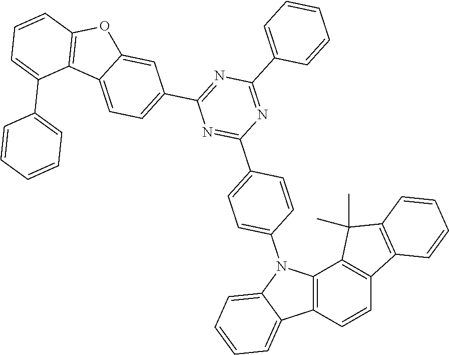 [SMILES: CC1(C)c2ccccc2-c2ccc3c4ccccc4n(-c4ccc(-c5nc(-c6ccccc6)nc(-c6ccc7c(c6)oc6cccc(-c8ccccc8)c67)n5)cc4)c3c21] | 301 |
| 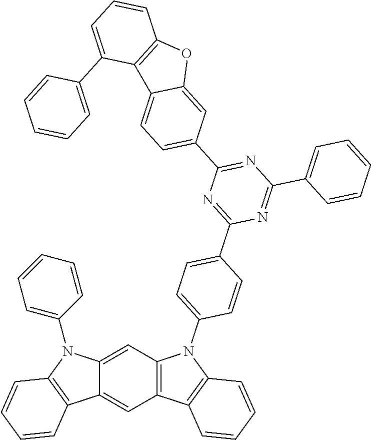 [SMILES: c1ccc(-c2nc(-c3ccc(-n4c5ccccc5c5cc6c7ccccc7n(-c7ccccc7)c6cc54)cc3)nc(-c3ccc4c(c3)oc3cccc(-c5ccccc5)c34)n2)cc1] | 302 |
| 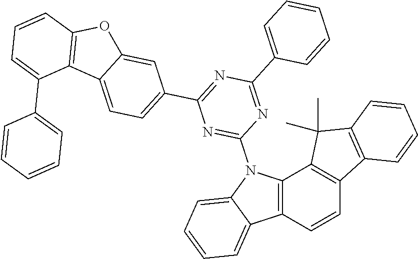 [SMILES: CC1(C)c2ccccc2-c2ccc3c4ccccc4n(-c4nc(-c5ccccc5)nc(-c5ccc6c(c5)oc5cccc(-c7ccccc7)c56)n4)c3c21] | 303 |
| 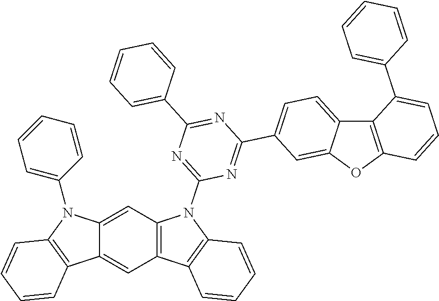 [SMILES: c1ccc(-c2nc(-c3ccc4c(c3)oc3cccc(-c5ccccc5)c34)nc(-n3c4ccccc4c4cc5c6ccccc6n(-c6ccccc6)c5cc43)n2)cc1] | 304 |
| 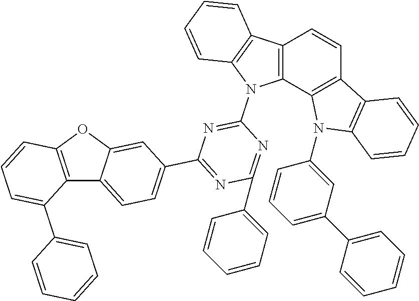 [SMILES: c1ccc(-c2cccc(-n3c4ccccc4c4ccc5c6ccccc6n(-c6nc(-c7ccccc7)nc(-c7ccc8c(c7)oc7cccc(-c9ccccc9)c78)n6)c5c43)c2)cc1] | 305 |
| 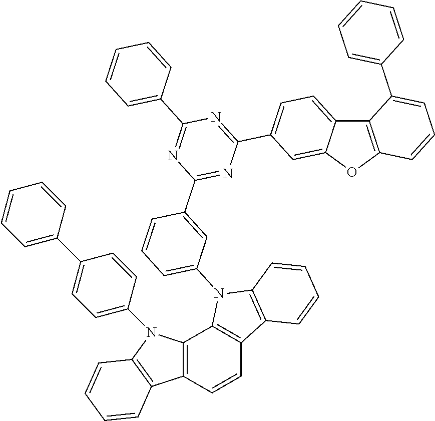 [SMILES: c1ccc(-c2ccc(-n3c4ccccc4c4ccc5c6ccccc6n(-c6cccc(-c7nc(-c8ccccc8)nc(-c8ccc9c(c8)oc8cccc(-c%10ccccc%10)c89)n7)c6)c5c43)cc2)cc1] | 306 |
| 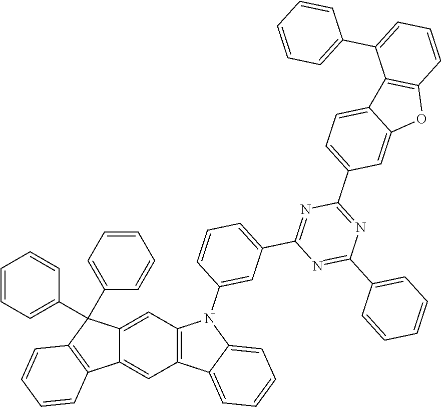 [SMILES: c1ccc(-c2nc(-c3cccc(-n4c5ccccc5c5cc6c(cc54)C(c4ccccc4)(c4ccccc4)c4ccccc4-6)c3)nc(-c3ccc4c(c3)oc3cccc(-c5ccccc5)c34)n2)cc1] | 307 |
| 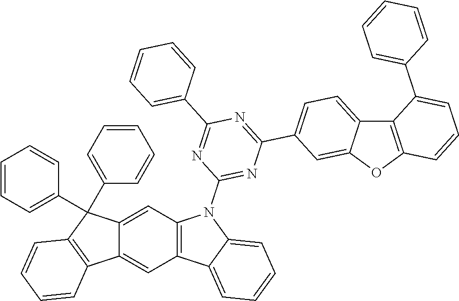 [SMILES: c1ccc(-c2nc(-c3ccc4c(c3)oc3cccc(-c5ccccc5)c34)nc(-n3c4ccccc4c4cc5c(cc43)C(c3ccccc3)(c3ccccc3)c3ccccc3-5)n2)cc1] | 308 |
| 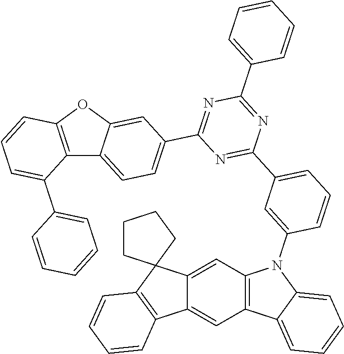 [SMILES: c1ccc(-c2nc(-c3cccc(-n4c5ccccc5c5cc6c(cc54)C4(CCCC4)c4ccccc4-6)c3)nc(-c3ccc4c(c3)oc3cccc(-c5ccccc5)c34)n2)cc1] | 309 |
| 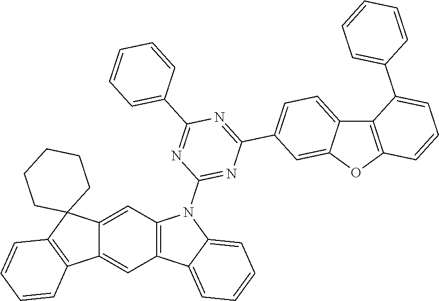 [SMILES: c1ccc(-c2nc(-c3ccc4c(c3)oc3cccc(-c5ccccc5)c34)nc(-n3c4ccccc4c4cc5c(cc43)C3(CCCCC3)c3ccccc3-5)n2)cc1] | 310 |
| 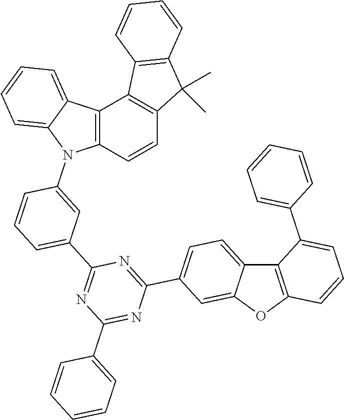 [SMILES: CC1(C)c2ccccc2-c2c1ccc1c2c2ccccc2n1-c1cccc(-c2nc(-c3ccccc3)nc(-c3ccc4c(c3)oc3cccc(-c5ccccc5)c34)n2)c1] | 311 |
| 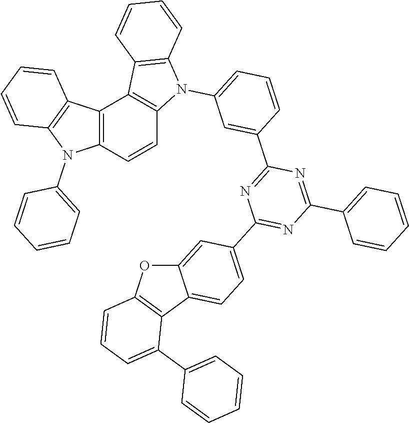 [SMILES: c1ccc(-c2nc(-c3cccc(-n4c5ccccc5c5c6c7ccccc7n(-c7ccccc7)c6ccc54)c3)nc(-c3ccc4c(c3)oc3cccc(-c5ccccc5)c34)n2)cc1] | 312 |
| 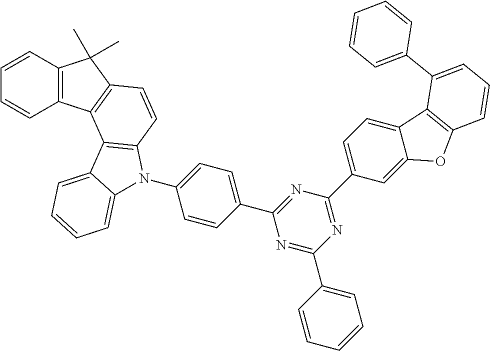 [SMILES: CC1(C)c2ccccc2-c2c1ccc1c2c2ccccc2n1-c1ccc(-c2nc(-c3ccccc3)nc(-c3ccc4c(c3)oc3cccc(-c5ccccc5)c34)n2)cc1] | 313 |
| 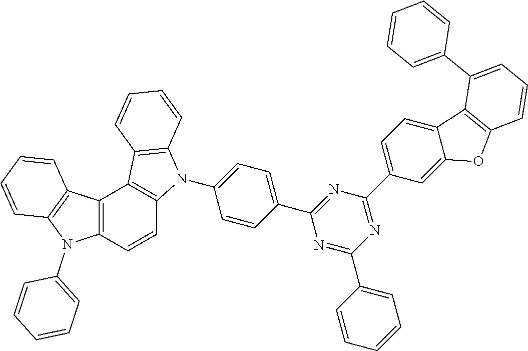 [SMILES: c1ccc(-c2nc(-c3ccc(-n4c5ccccc5c5c6c7ccccc7n(-c7ccccc7)c6ccc54)cc3)nc(-c3ccc4c(c3)oc3cccc(-c5ccccc5)c34)n2)cc1] | 314 |
| 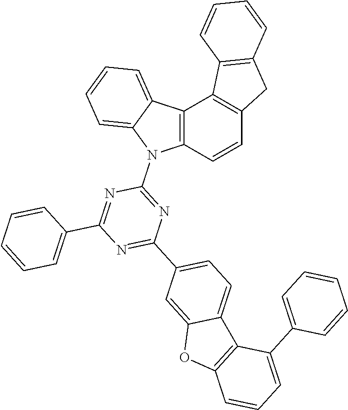 [SMILES: c1ccc(-c2nc(-c3ccc4c(c3)oc3cccc(-c5ccccc5)c34)nc(-n3c4ccccc4c4c5c(ccc43)Cc3ccccc3-5)n2)cc1] | 315 |
| 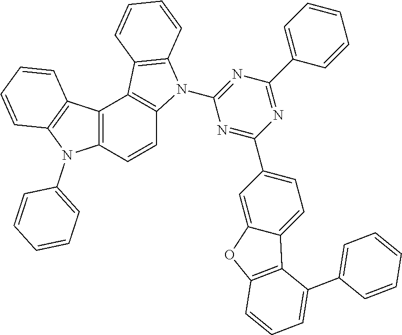 [SMILES: c1ccc(-c2nc(-c3ccc4c(c3)oc3cccc(-c5ccccc5)c34)nc(-n3c4ccccc4c4c5c6ccccc6n(-c6ccccc6)c5ccc43)n2)cc1] | 316 |
| 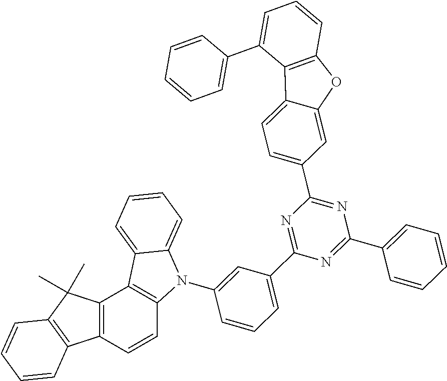 [SMILES: CC1(C)c2ccccc2-c2ccc3c(c21)c1ccccc1n3-c1cccc(-c2nc(-c3ccccc3)nc(-c3ccc4c(c3)oc3cccc(-c5ccccc5)c34)n2)c1] | 317 |
| 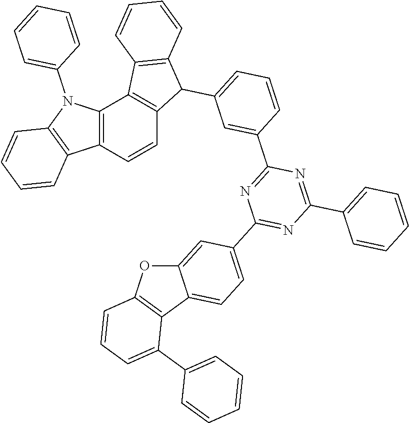 [SMILES: c1ccc(-c2nc(-c3cccc(C4c5ccccc5-c5c4ccc4c6ccccc6n(-c6ccccc6)c54)c3)nc(-c3ccc4c(c3)oc3cccc(-c5ccccc5)c34)n2)cc1] | 318 |
| 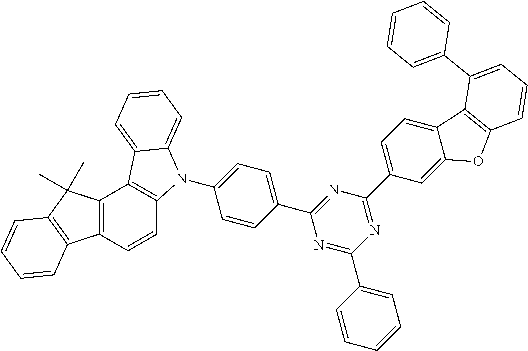 [SMILES: CC1(C)c2ccccc2-c2ccc3c(c21)c1ccccc1n3-c1ccc(-c2nc(-c3ccccc3)nc(-c3ccc4c(c3)oc3cccc(-c5ccccc5)c34)n2)cc1] | 319 |
| 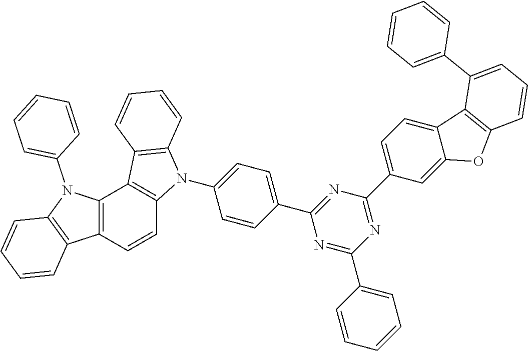 [SMILES: c1ccc(-c2nc(-c3ccc(-n4c5ccccc5c5c4ccc4c6ccccc6n(-c6ccccc6)c45)cc3)nc(-c3ccc4c(c3)oc3cccc(-c5ccccc5)c34)n2)cc1] | 320 |
| 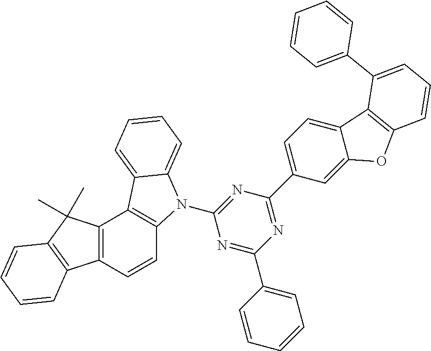 [SMILES: CC1(C)c2ccccc2-c2ccc3c(c21)c1ccccc1n3-c1nc(-c2ccccc2)nc(-c2ccc3c(c2)oc2cccc(-c4ccccc4)c23)n1] | 321 |
| 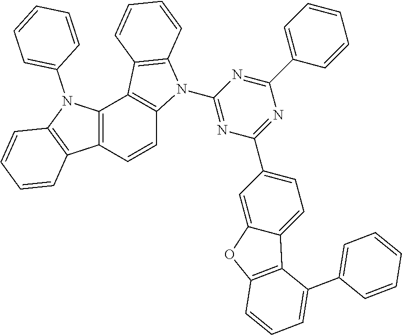 [SMILES: c1ccc(-c2nc(-c3ccc4c(c3)oc3cccc(-c5ccccc5)c34)nc(-n3c4ccccc4c4c3ccc3c5ccccc5n(-c5ccccc5)c34)n2)cc1] | 322 |
| 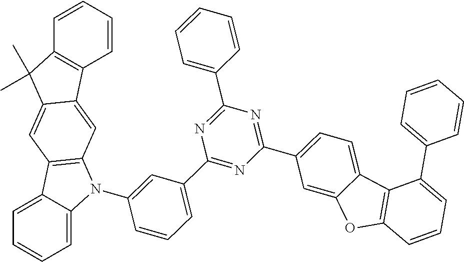 [SMILES: CC1(C)c2ccccc2-c2cc3c(cc21)c1ccccc1n3-c1cccc(-c2nc(-c3ccccc3)nc(-c3ccc4c(c3)oc3cccc(-c5ccccc5)c34)n2)c1] | 323 |
| 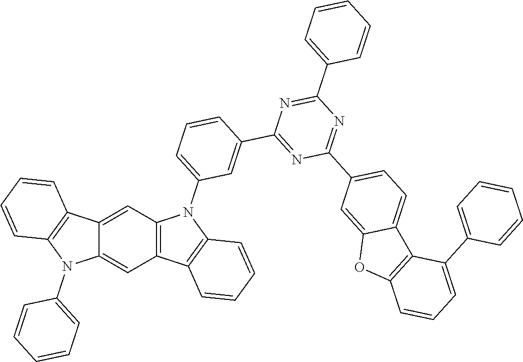 [SMILES: c1ccc(-c2nc(-c3cccc(-n4c5ccccc5c5cc6c(cc54)c4ccccc4n6-c4ccccc4)c3)nc(-c3ccc4c(c3)oc3cccc(-c5ccccc5)c34)n2)cc1] | 324 |
| 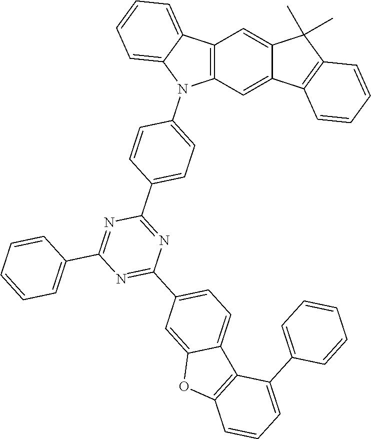 [SMILES: CC1(C)c2ccccc2-c2cc3c(cc21)c1ccccc1n3-c1ccc(-c2nc(-c3ccccc3)nc(-c3ccc4c(c3)oc3cccc(-c5ccccc5)c34)n2)cc1] | 325 |
| 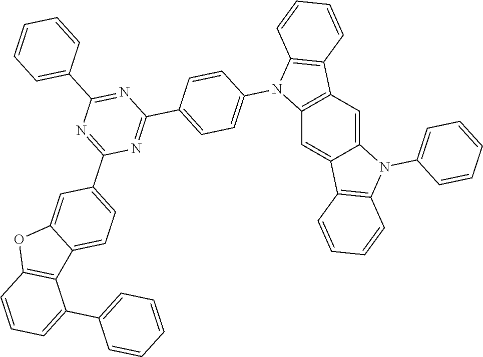 [SMILES: c1ccc(-c2nc(-c3ccc(-n4c5ccccc5c5cc6c(cc54)c4ccccc4n6-c4ccccc4)cc3)nc(-c3ccc4c(c3)oc3cccc(-c5ccccc5)c34)n2)cc1] | 326 |
| 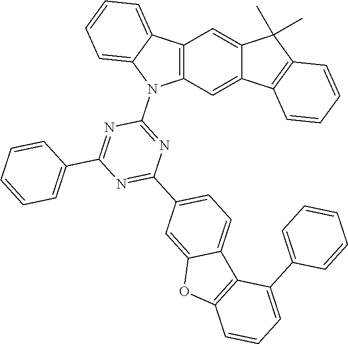 [SMILES: CC1(C)c2ccccc2-c2cc3c(cc21)c1ccccc1n3-c1nc(-c2ccccc2)nc(-c2ccc3c(c2)oc2cccc(-c4ccccc4)c23)n1] | 327 |
| 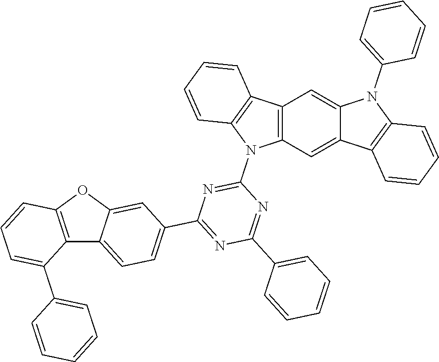 [SMILES: c1ccc(-c2nc(-c3ccc4c(c3)oc3cccc(-c5ccccc5)c34)nc(-n3c4ccccc4c4cc5c(cc43)c3ccccc3n5-c3ccccc3)n2)cc1] | 328 |
| 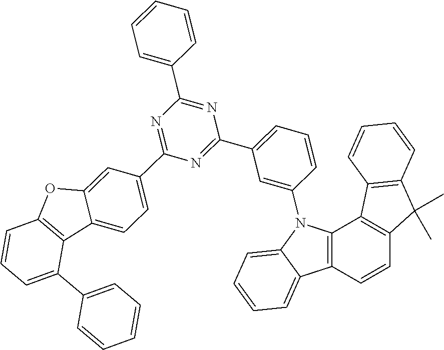 [SMILES: CC1(C)c2ccccc2-c2c1ccc1c3ccccc3n(-c3cccc(-c4nc(-c5ccccc5)nc(-c5ccc6c(c5)oc5cccc(-c7ccccc7)c56)n4)c3)c21] | 329 |
| 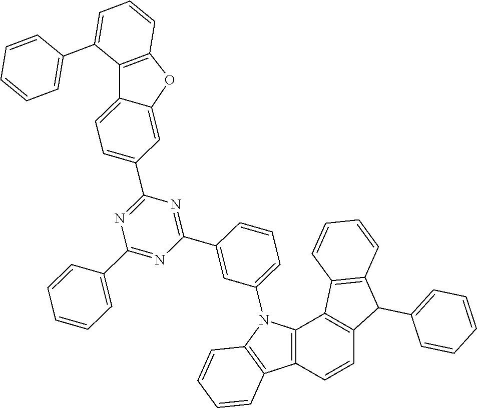 [SMILES: c1ccc(-c2nc(-c3cccc(-n4c5ccccc5c5ccc6c(c54)-c4ccccc4C6c4ccccc4)c3)nc(-c3ccc4c(c3)oc3cccc(-c5ccccc5)c34)n2)cc1] | 330 |
| 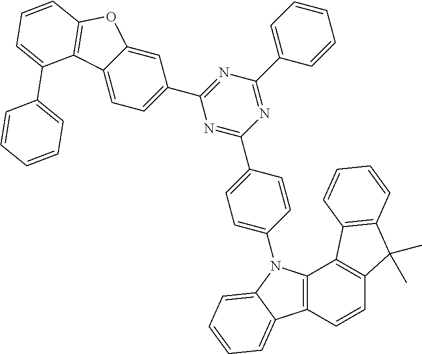 [SMILES: CC1(C)c2ccccc2-c2c1ccc1c3ccccc3n(-c3ccc(-c4nc(-c5ccccc5)nc(-c5ccc6c(c5)oc5cccc(-c7ccccc7)c56)n4)cc3)c21] | 331 |
| 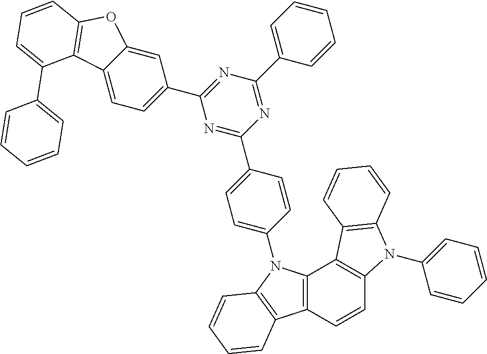 [SMILES: c1ccc(-c2nc(-c3ccc(-n4c5ccccc5c5ccc6c(c7ccccc7n6-c6ccccc6)c54)cc3)nc(-c3ccc4c(c3)oc3cccc(-c5ccccc5)c34)n2)cc1] | 332 |
| 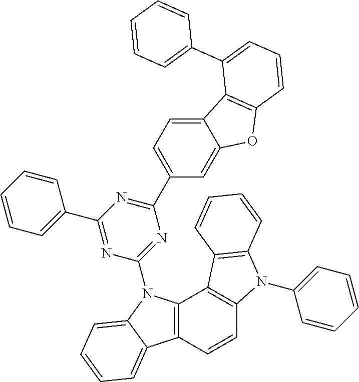 [SMILES: c1ccc(-c2nc(-c3ccc4c(c3)oc3cccc(-c5ccccc5)c34)nc(-n3c4ccccc4c4ccc5c(c6ccccc6n5-c5ccccc5)c43)n2)cc1] | 333 |
| 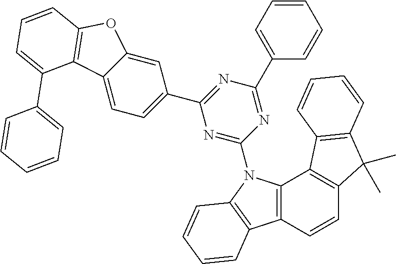 [SMILES: CC1(C)c2ccccc2-c2c1ccc1c3ccccc3n(-c3nc(-c4ccccc4)nc(-c4ccc5c(c4)oc4cccc(-c6ccccc6)c45)n3)c21] | 334 |
| 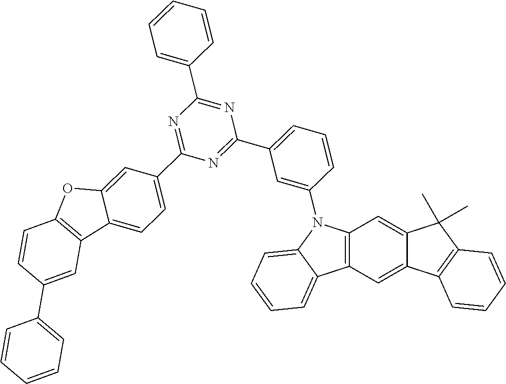 [SMILES: CC1(C)c2ccccc2-c2cc3c4ccccc4n(-c4cccc(-c5nc(-c6ccccc6)nc(-c6ccc7c(c6)oc6ccc(-c8ccccc8)cc67)n5)c4)c3cc21] | 335 |
| 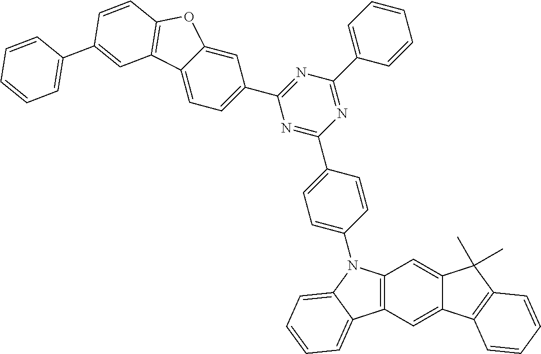 [SMILES: CC1(C)c2ccccc2-c2cc3c4ccccc4n(-c4ccc(-c5nc(-c6ccccc6)nc(-c6ccc7c(c6)oc6ccc(-c8ccccc8)cc67)n5)cc4)c3cc21] | 336 |
| 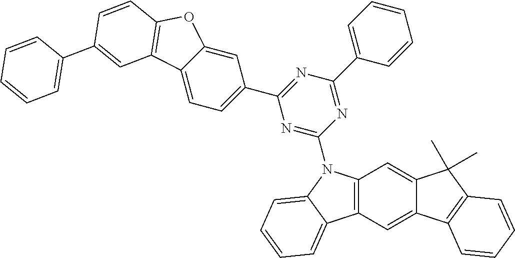 [SMILES: CC1(C)c2ccccc2-c2cc3c4ccccc4n(-c4nc(-c5ccccc5)nc(-c5ccc6c(c5)oc5ccc(-c7ccccc7)cc56)n4)c3cc21] | 337 |
| 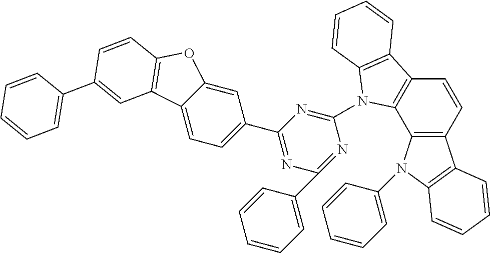 [SMILES: c1ccc(-c2ccc3oc4cc(-c5nc(-c6ccccc6)nc(-n6c7ccccc7c7ccc8c9ccccc9n(-c9ccccc9)c8c76)n5)ccc4c3c2)cc1] | 338 |
| 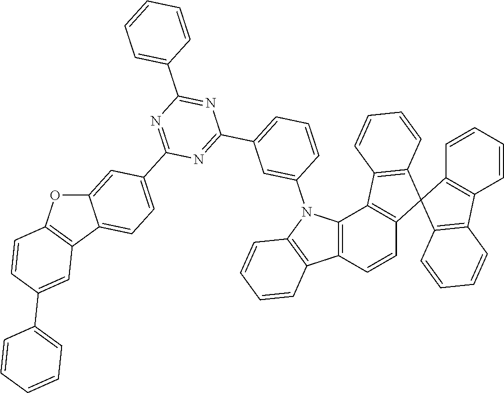 [SMILES: c1ccc(-c2ccc3oc4cc(-c5nc(-c6ccccc6)nc(-c6cccc(-n7c8ccccc8c8ccc9c(c87)-c7ccccc7C97c8ccccc8-c8ccccc87)c6)n5)ccc4c3c2)cc1] | 339 |
| 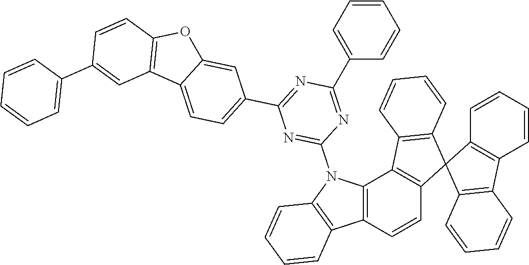 [SMILES: c1ccc(-c2ccc3oc4cc(-c5nc(-c6ccccc6)nc(-n6c7ccccc7c7ccc8c(c76)-c6ccccc6C86c7ccccc7-c7ccccc76)n5)ccc4c3c2)cc1] | 340 |
| 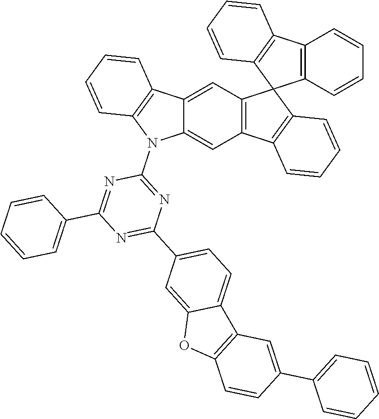 [SMILES: c1ccc(-c2ccc3oc4cc(-c5nc(-c6ccccc6)nc(-n6c7ccccc7c7cc8c(cc76)-c6ccccc6C86c7ccccc7-c7ccccc76)n5)ccc4c3c2)cc1] | 341 |
| 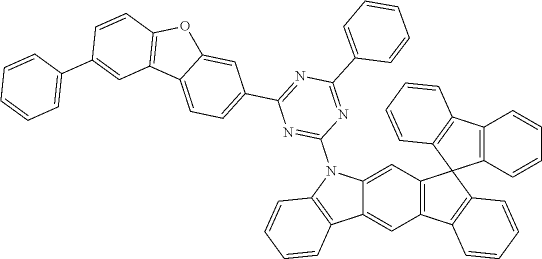 [SMILES: c1ccc(-c2ccc3oc4cc(-c5nc(-c6ccccc6)nc(-n6c7ccccc7c7cc8c(cc76)C6(c7ccccc7-c7ccccc76)c6ccccc6-8)n5)ccc4c3c2)cc1] | 342 |
| 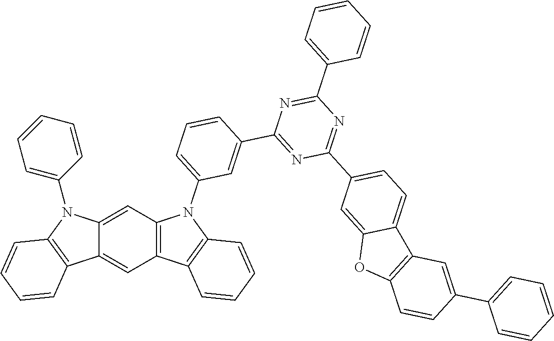 [SMILES: c1ccc(-c2ccc3oc4cc(-c5nc(-c6ccccc6)nc(-c6cccc(-n7c8ccccc8c8cc9c%10ccccc%10n(-c%10ccccc%10)c9cc87)c6)n5)ccc4c3c2)cc1] | 343 |
| 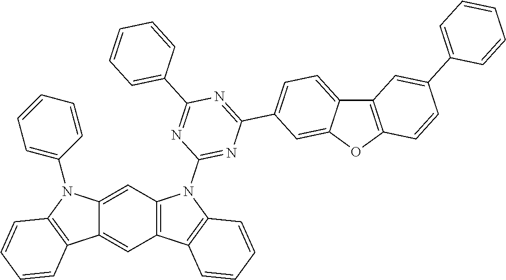 [SMILES: c1ccc(-c2ccc3oc4cc(-c5nc(-c6ccccc6)nc(-n6c7ccccc7c7cc8c9ccccc9n(-c9ccccc9)c8cc76)n5)ccc4c3c2)cc1] | 344 |
| 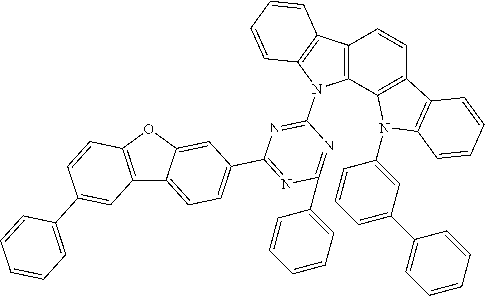 [SMILES: c1ccc(-c2cccc(-n3c4ccccc4c4ccc5c6ccccc6n(-c6nc(-c7ccccc7)nc(-c7ccc8c(c7)oc7ccc(-c9ccccc9)cc78)n6)c5c43)c2)cc1] | 345 |
| 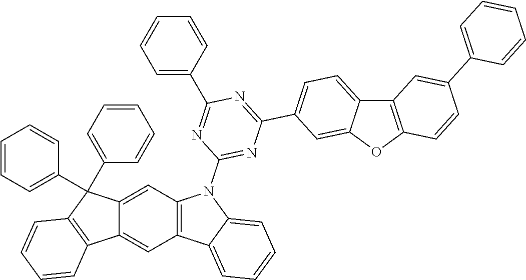 [SMILES: c1ccc(-c2ccc3oc4cc(-c5nc(-c6ccccc6)nc(-n6c7ccccc7c7cc8c(cc76)C(c6ccccc6)(c6ccccc6)c6ccccc6-8)n5)ccc4c3c2)cc1] | 346 |
| 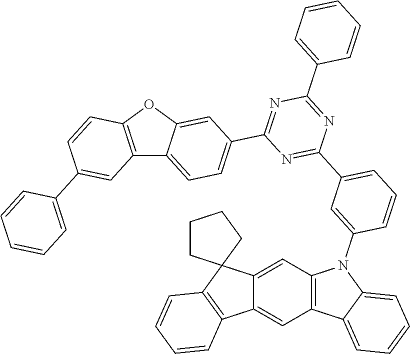 [SMILES: c1ccc(-c2ccc3oc4cc(-c5nc(-c6ccccc6)nc(-c6cccc(-n7c8ccccc8c8cc9c(cc87)C7(CCCC7)c7ccccc7-9)c6)n5)ccc4c3c2)cc1] | 347 |
| 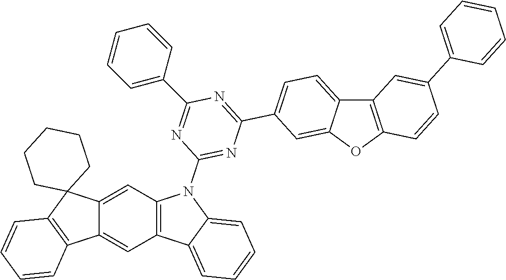 [SMILES: c1ccc(-c2ccc3oc4cc(-c5nc(-c6ccccc6)nc(-n6c7ccccc7c7cc8c(cc76)C6(CCCCC6)c6ccccc6-8)n5)ccc4c3c2)cc1] | 348 |
| 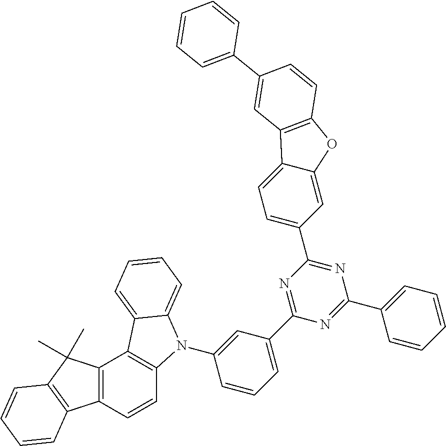 [SMILES: CC1(C)c2ccccc2-c2ccc3c(c21)c1ccccc1n3-c1cccc(-c2nc(-c3ccccc3)nc(-c3ccc4c(c3)oc3ccc(-c5ccccc5)cc34)n2)c1] | 349 |
| 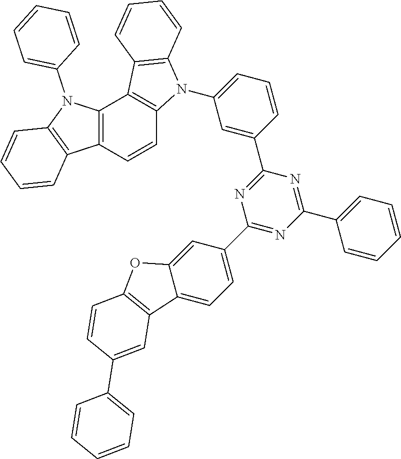 [SMILES: c1ccc(-c2ccc3oc4cc(-c5nc(-c6ccccc6)nc(-c6cccc(-n7c8ccccc8c8c7ccc7c9ccccc9n(-c9ccccc9)c78)c6)n5)ccc4c3c2)cc1] | 350 |
| 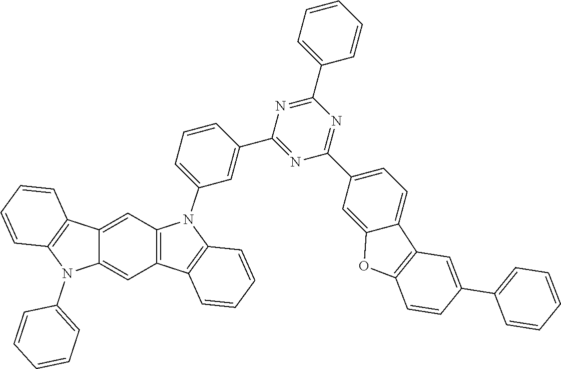 [SMILES: c1ccc(-c2ccc3oc4cc(-c5nc(-c6ccccc6)nc(-c6cccc(-n7c8ccccc8c8cc9c(cc87)c7ccccc7n9-c7ccccc7)c6)n5)ccc4c3c2)cc1] | 351 |
| 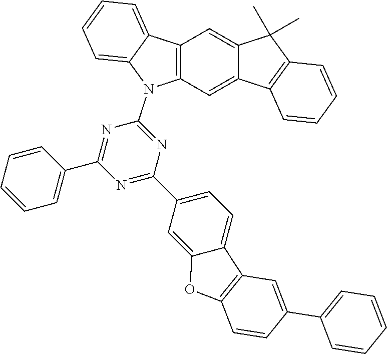 [SMILES: CC1(C)c2ccccc2-c2cc3c(cc21)c1ccccc1n3-c1nc(-c2ccccc2)nc(-c2ccc3c(c2)oc2ccc(-c4ccccc4)cc23)n1] | 352 |
| 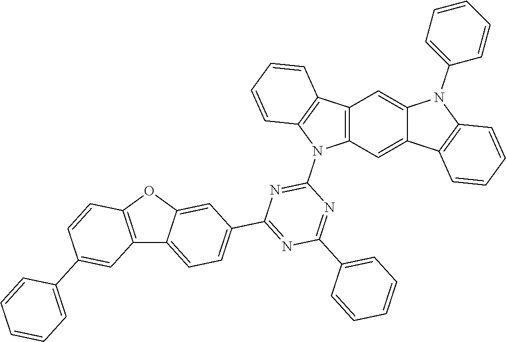 [SMILES: c1ccc(-c2ccc3oc4cc(-c5nc(-c6ccccc6)nc(-n6c7ccccc7c7cc8c(cc76)c6ccccc6n8-c6ccccc6)n5)ccc4c3c2)cc1] | 353 |
| 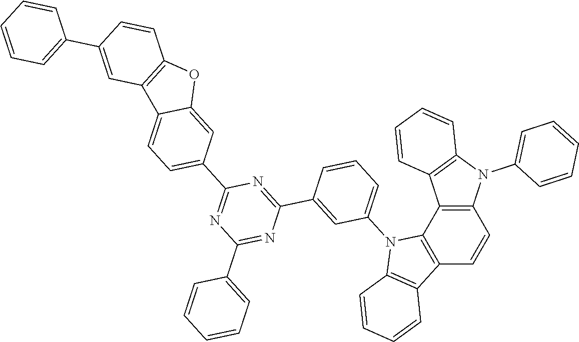 [SMILES: c1ccc(-c2ccc3oc4cc(-c5nc(-c6ccccc6)nc(-c6cccc(-n7c8ccccc8c8ccc9c(c%10ccccc%10n9-c9ccccc9)c87)c6)n5)ccc4c3c2)cc1] | 354 |
| 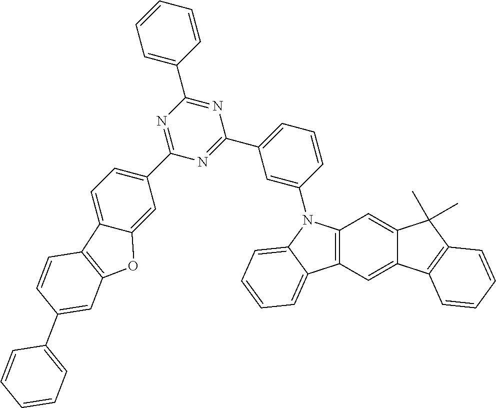 [SMILES: CC1(C)c2ccccc2-c2cc3c4ccccc4n(-c4cccc(-c5nc(-c6ccccc6)nc(-c6ccc7c(c6)oc6cc(-c8ccccc8)ccc67)n5)c4)c3cc21] | 355 |
| 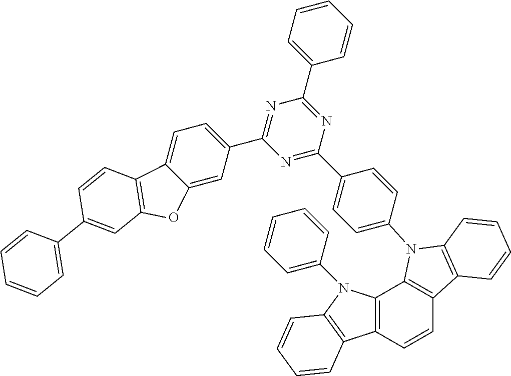 [SMILES: c1ccc(-c2ccc3c(c2)oc2cc(-c4nc(-c5ccccc5)nc(-c5ccc(-n6c7ccccc7c7ccc8c9ccccc9n(-c9ccccc9)c8c76)cc5)n4)ccc23)cc1] | 356 |
| 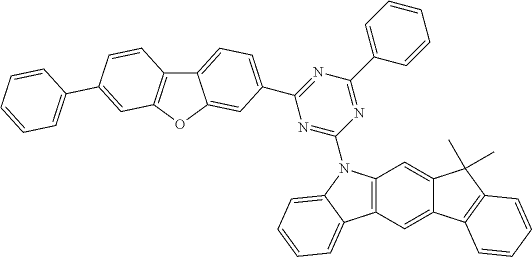 [SMILES: CC1(C)c2ccccc2-c2cc3c4ccccc4n(-c4nc(-c5ccccc5)nc(-c5ccc6c(c5)oc5cc(-c7ccccc7)ccc56)n4)c3cc21] | 357 |
| 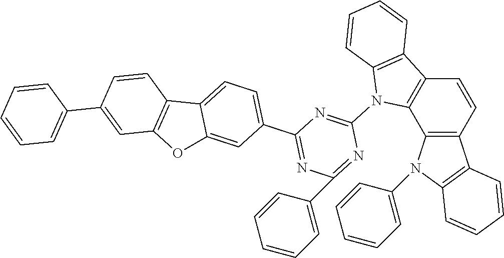 [SMILES: c1ccc(-c2ccc3c(c2)oc2cc(-c4nc(-c5ccccc5)nc(-n5c6ccccc6c6ccc7c8ccccc8n(-c8ccccc8)c7c65)n4)ccc23)cc1] | 358 |
| 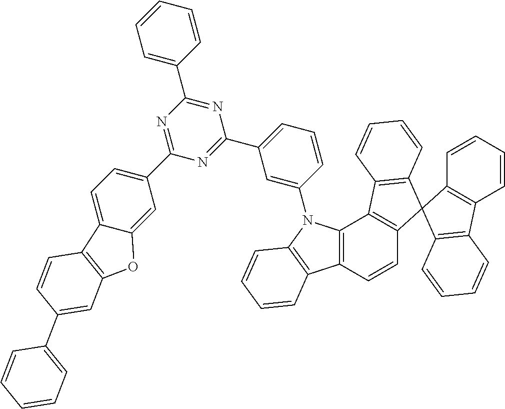 [SMILES: c1ccc(-c2ccc3c(c2)oc2cc(-c4nc(-c5ccccc5)nc(-c5cccc(-n6c7ccccc7c7ccc8c(c76)-c6ccccc6C86c7ccccc7-c7ccccc76)c5)n4)ccc23)cc1] | 359 |
| 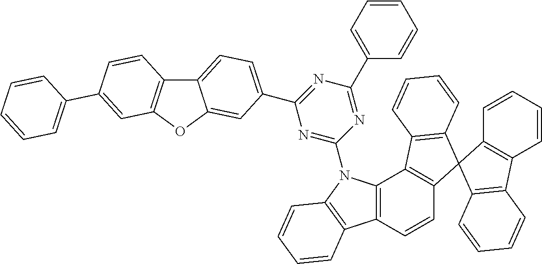 [SMILES: c1ccc(-c2ccc3c(c2)oc2cc(-c4nc(-c5ccccc5)nc(-n5c6ccccc6c6ccc7c(c65)-c5ccccc5C75c6ccccc6-c6ccccc65)n4)ccc23)cc1] | 360 |
| 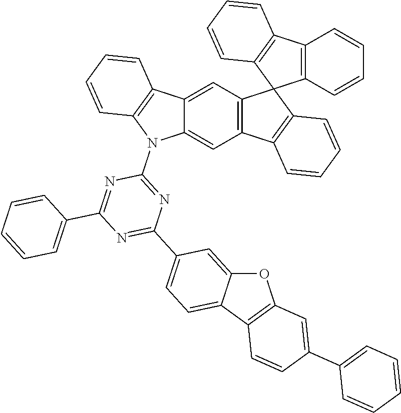 [SMILES: c1ccc(-c2ccc3c(c2)oc2cc(-c4nc(-c5ccccc5)nc(-n5c6ccccc6c6cc7c(cc65)-c5ccccc5C75c6ccccc6-c6ccccc65)n4)ccc23)cc1] | 361 |
| 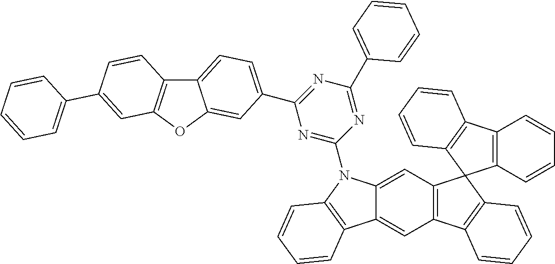 [SMILES: c1ccc(-c2ccc3c(c2)oc2cc(-c4nc(-c5ccccc5)nc(-n5c6ccccc6c6cc7c(cc65)C5(c6ccccc6-c6ccccc65)c5ccccc5-7)n4)ccc23)cc1] | 362 |
| 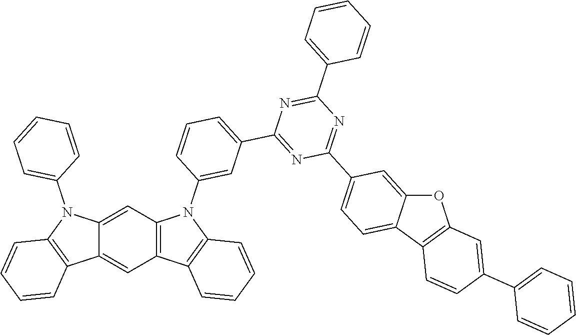 [SMILES: c1ccc(-c2ccc3c(c2)oc2cc(-c4nc(-c5ccccc5)nc(-c5cccc(-n6c7ccccc7c7cc8c9ccccc9n(-c9ccccc9)c8cc76)c5)n4)ccc23)cc1] | 363 |
| 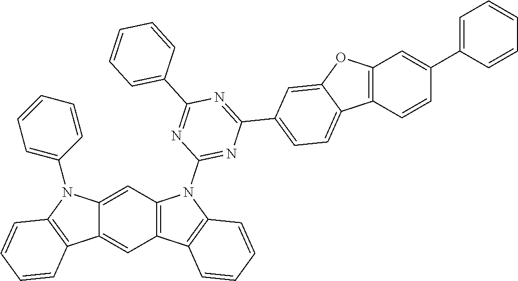 [SMILES: c1ccc(-c2ccc3c(c2)oc2cc(-c4nc(-c5ccccc5)nc(-n5c6ccccc6c6cc7c8ccccc8n(-c8ccccc8)c7cc65)n4)ccc23)cc1] | 364 |
| 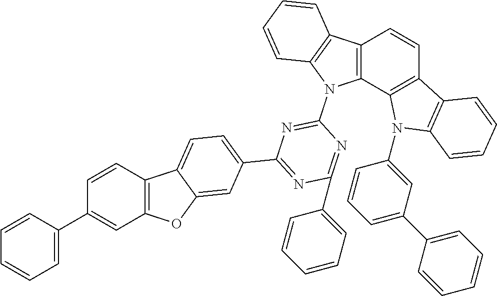 [SMILES: c1ccc(-c2cccc(-n3c4ccccc4c4ccc5c6ccccc6n(-c6nc(-c7ccccc7)nc(-c7ccc8c(c7)oc7cc(-c9ccccc9)ccc78)n6)c5c43)c2)cc1] | 365 |
| 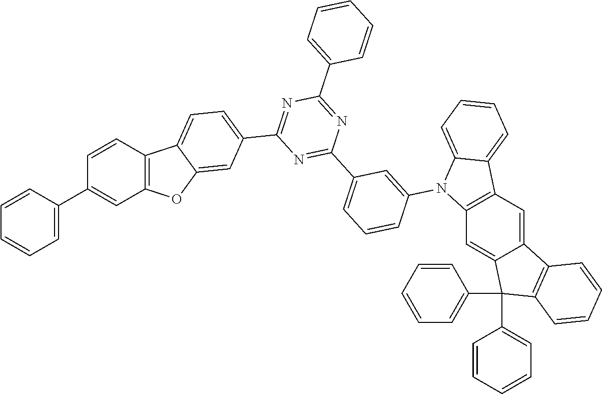 [SMILES: c1ccc(-c2ccc3c(c2)oc2cc(-c4nc(-c5ccccc5)nc(-c5cccc(-n6c7ccccc7c7cc8c(cc76)C(c6ccccc6)(c6ccccc6)c6ccccc6-8)c5)n4)ccc23)cc1] | 366 |
| 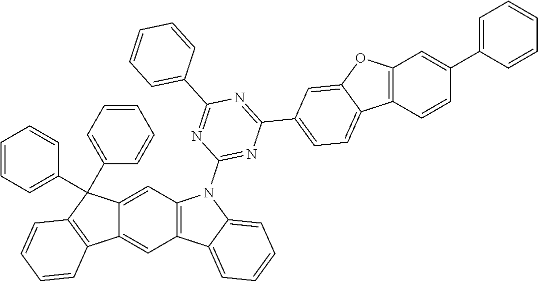 [SMILES: c1ccc(-c2ccc3c(c2)oc2cc(-c4nc(-c5ccccc5)nc(-n5c6ccccc6c6cc7c(cc65)C(c5ccccc5)(c5ccccc5)c5ccccc5-7)n4)ccc23)cc1] | 367 |
| 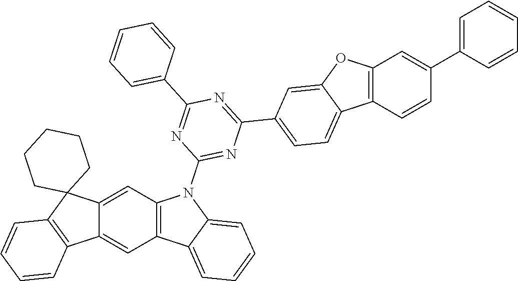 [SMILES: c1ccc(-c2ccc3c(c2)oc2cc(-c4nc(-c5ccccc5)nc(-n5c6ccccc6c6cc7c(cc65)C5(CCCCC5)c5ccccc5-7)n4)ccc23)cc1] | 368 |
| 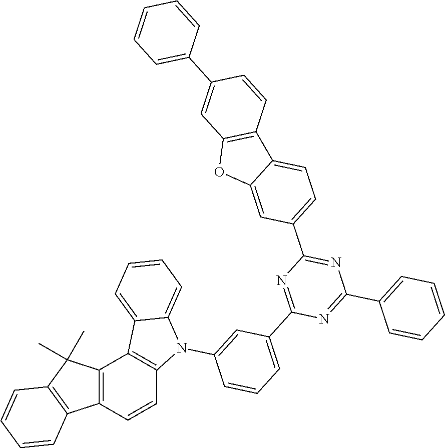 [SMILES: CC1(C)c2ccccc2-c2ccc3c(c21)c1ccccc1n3-c1cccc(-c2nc(-c3ccccc3)nc(-c3ccc4c(c3)oc3cc(-c5ccccc5)ccc34)n2)c1] | 369 |
| 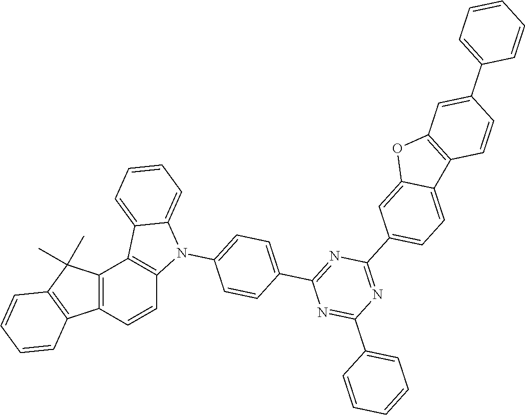 [SMILES: CC1(C)c2ccccc2-c2ccc3c(c21)c1ccccc1n3-c1ccc(-c2nc(-c3ccccc3)nc(-c3ccc4c(c3)oc3cc(-c5ccccc5)ccc34)n2)cc1] | 370 |
| 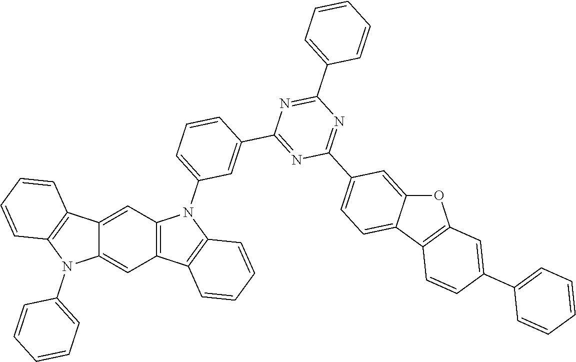 [SMILES: c1ccc(-c2ccc3c(c2)oc2cc(-c4nc(-c5ccccc5)nc(-c5cccc(-n6c7ccccc7c7cc8c(cc76)c6ccccc6n8-c6ccccc6)c5)n4)ccc23)cc1] | 371 |
| 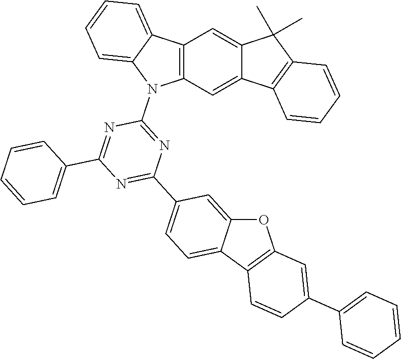 [SMILES: CC1(C)c2ccccc2-c2cc3c(cc21)c1ccccc1n3-c1nc(-c2ccccc2)nc(-c2ccc3c(c2)oc2cc(-c4ccccc4)ccc23)n1] | 372 |
| 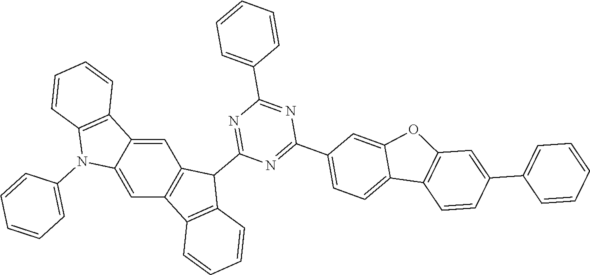 [SMILES: c1ccc(-c2ccc3c(c2)oc2cc(-c4nc(-c5ccccc5)nc(C5c6ccccc6-c6cc7c(cc65)c5ccccc5n7-c5ccccc5)n4)ccc23)cc1] | 373 |
| 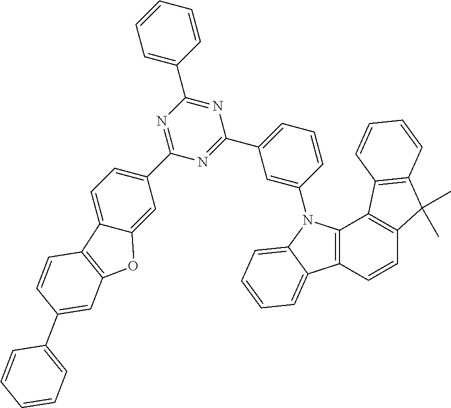 [SMILES: CC1(C)c2ccccc2-c2c1ccc1c3ccccc3n(-c3cccc(-c4nc(-c5ccccc5)nc(-c5ccc6c(c5)oc5cc(-c7ccccc7)ccc56)n4)c3)c21] | 374 |
| 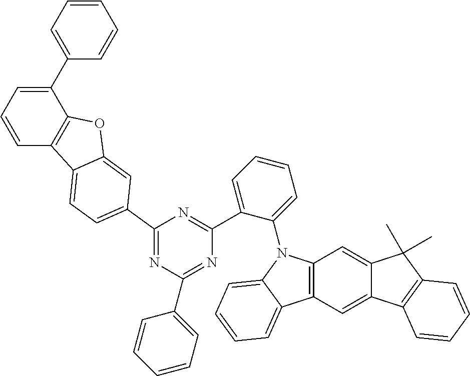 [SMILES: CC1(C)c2ccccc2-c2cc3c4ccccc4n(-c4ccccc4-c4nc(-c5ccccc5)nc(-c5ccc6c(c5)oc5c(-c7ccccc7)cccc56)n4)c3cc21] | 375 |
| 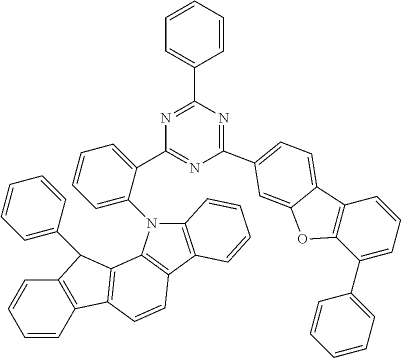 [SMILES: c1ccc(-c2nc(-c3ccc4c(c3)oc3c(-c5ccccc5)cccc34)nc(-c3ccccc3-n3c4ccccc4c4ccc5c(c43)C(c3ccccc3)c3ccccc3-5)n2)cc1] | 376 |
| 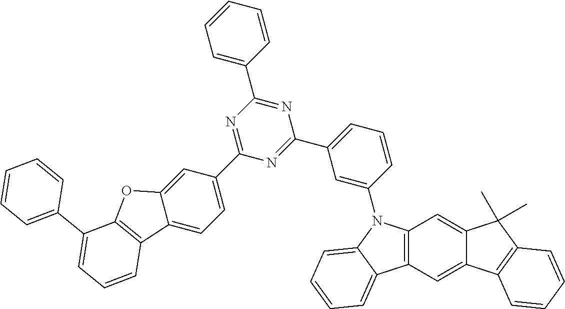 [SMILES: CC1(C)c2ccccc2-c2cc3c4ccccc4n(-c4cccc(-c5nc(-c6ccccc6)nc(-c6ccc7c(c6)oc6c(-c8ccccc8)cccc67)n5)c4)c3cc21] | 377 |
| 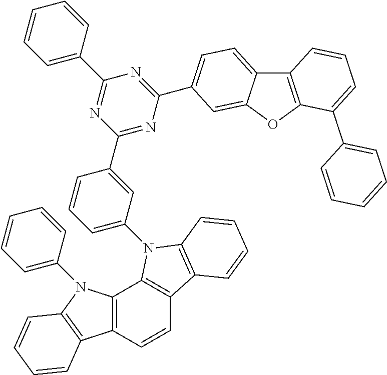 [SMILES: c1ccc(-c2nc(-c3cccc(-n4c5ccccc5c5ccc6c7ccccc7n(-c7ccccc7)c6c54)c3)nc(-c3ccc4c(c3)oc3c(-c5ccccc5)cccc34)n2)cc1] | 378 |
| 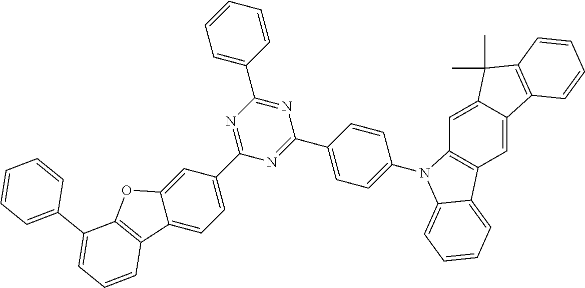 [SMILES: CC1(C)c2ccccc2-c2cc3c4ccccc4n(-c4ccc(-c5nc(-c6ccccc6)nc(-c6ccc7c(c6)oc6c(-c8ccccc8)cccc67)n5)cc4)c3cc21] | 379 |
| 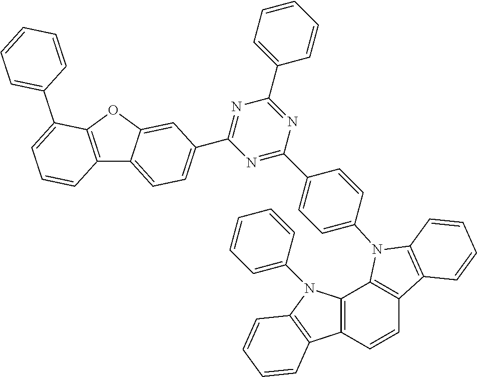 [SMILES: c1ccc(-c2nc(-c3ccc(-n4c5ccccc5c5ccc6c7ccccc7n(-c7ccccc7)c6c54)cc3)nc(-c3ccc4c(c3)oc3c(-c5ccccc5)cccc34)n2)cc1] | 380 |
| 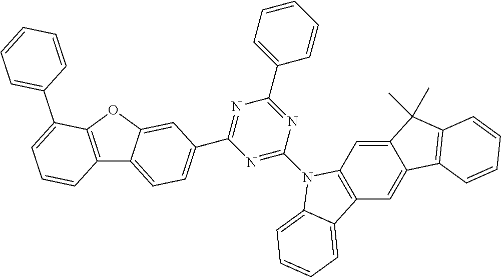 [SMILES: CC1(C)c2ccccc2-c2cc3c4ccccc4n(-c4nc(-c5ccccc5)nc(-c5ccc6c(c5)oc5c(-c7ccccc7)cccc56)n4)c3cc21] | 381 |
| 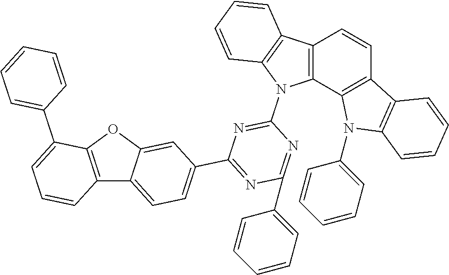 [SMILES: c1ccc(-c2nc(-c3ccc4c(c3)oc3c(-c5ccccc5)cccc34)nc(-n3c4ccccc4c4ccc5c6ccccc6n(-c6ccccc6)c5c43)n2)cc1] | 382 |
| 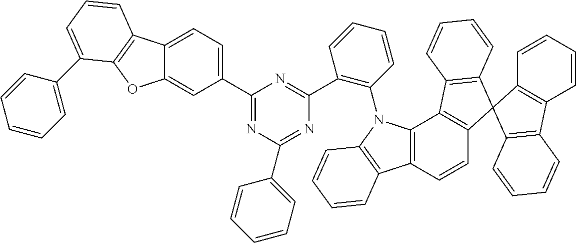 [SMILES: c1ccc(-c2nc(-c3ccc4c(c3)oc3c(-c5ccccc5)cccc34)nc(-c3ccccc3-n3c4ccccc4c4ccc5c(c43)-c3ccccc3C53c4ccccc4-c4ccccc43)n2)cc1] | 383 |
| 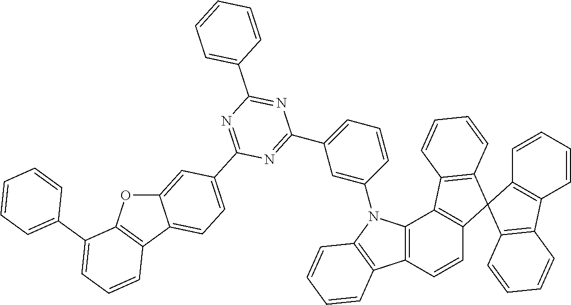 [SMILES: c1ccc(-c2nc(-c3cccc(-n4c5ccccc5c5ccc6c(c54)-c4ccccc4C64c5ccccc5-c5ccccc54)c3)nc(-c3ccc4c(c3)oc3c(-c5ccccc5)cccc34)n2)cc1] | 384 |
| 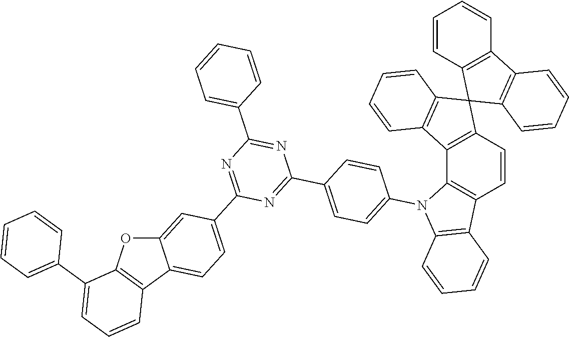 [SMILES: c1ccc(-c2nc(-c3ccc(-n4c5ccccc5c5ccc6c(c54)-c4ccccc4C64c5ccccc5-c5ccccc54)cc3)nc(-c3ccc4c(c3)oc3c(-c5ccccc5)cccc34)n2)cc1] | 385 |
| 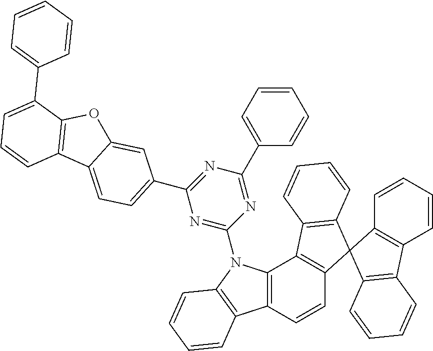 [SMILES: c1ccc(-c2nc(-c3ccc4c(c3)oc3c(-c5ccccc5)cccc34)nc(-n3c4ccccc4c4ccc5c(c43)-c3ccccc3C53c4ccccc4-c4ccccc43)n2)cc1] | 386 |
| 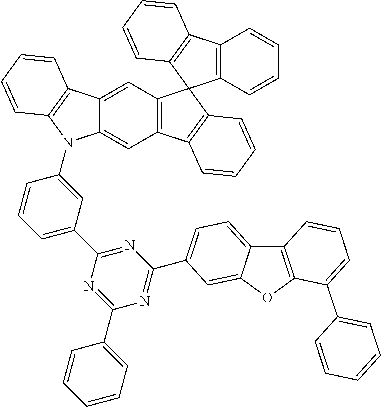 | 387 |
| 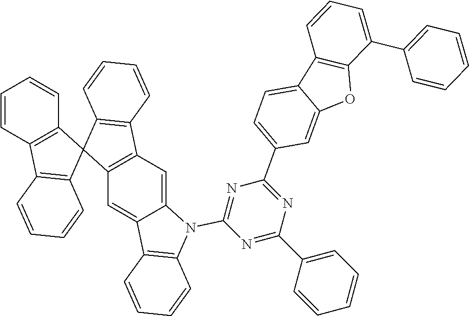 [SMILES: c1ccc(-c2nc(-c3ccc4c(c3)oc3c(-c5ccccc5)cccc34)nc(-n3c4ccccc4c4cc5c(cc43)-c3ccccc3C53c4ccccc4-c4ccccc43)n2)cc1] | 388 |
| 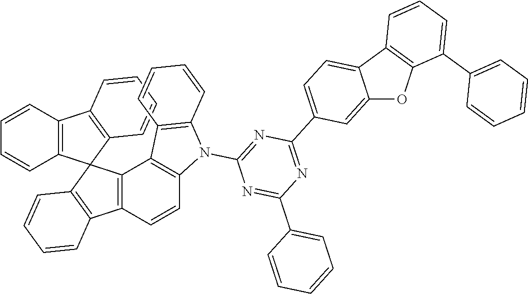 [SMILES: c1ccc(-c2nc(-c3ccc4c(c3)oc3c(-c5ccccc5)cccc34)nc(-n3c4ccccc4c4c5c(ccc43)-c3ccccc3C53c4ccccc4-c4ccccc43)n2)cc1] | 389 |
| 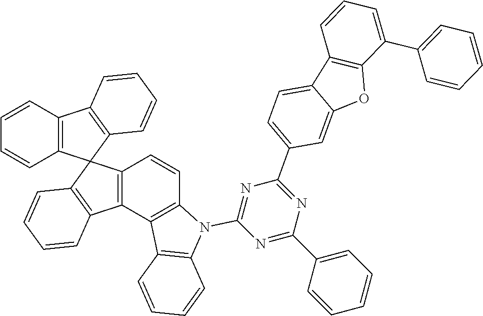 [SMILES: c1ccc(-c2nc(-c3ccc4c(c3)oc3c(-c5ccccc5)cccc34)nc(-n3c4ccccc4c4c5c(ccc43)C3(c4ccccc4-c4ccccc43)c3ccccc3-5)n2)cc1] | 390 |
| 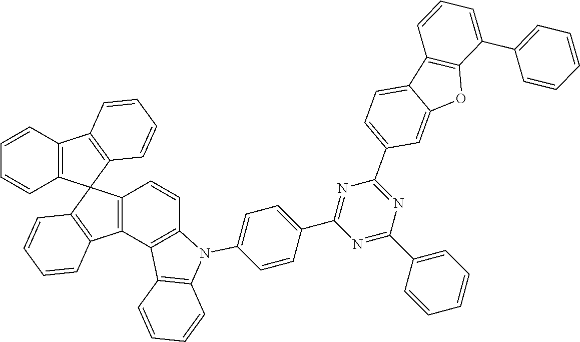 [SMILES: c1ccc(-c2nc(-c3ccc(-n4c5ccccc5c5c6c(ccc54)C4(c5ccccc5-c5ccccc54)c4ccccc4-6)cc3)nc(-c3ccc4c(c3)oc3c(-c5ccccc5)cccc34)n2)cc1] | 391 |
| 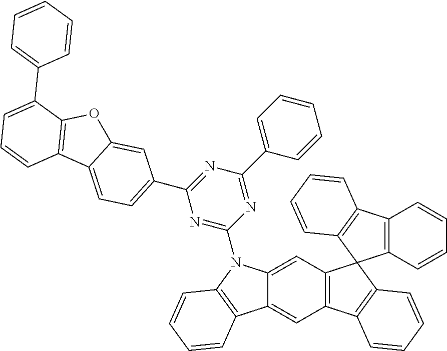 [SMILES: c1ccc(-c2nc(-c3ccc4c(c3)oc3c(-c5ccccc5)cccc34)nc(-n3c4ccccc4c4cc5c(cc43)C3(c4ccccc4-c4ccccc43)c3ccccc3-5)n2)cc1] | 392 |
| 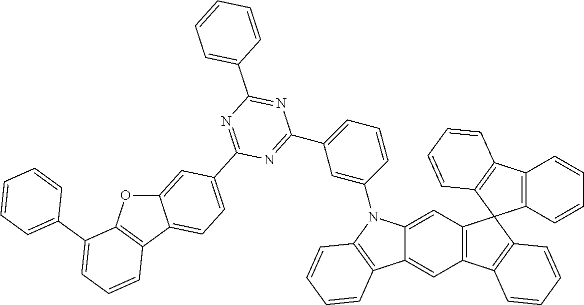 [SMILES: c1ccc(-c2nc(-c3cccc(-n4c5ccccc5c5cc6c(cc54)C4(c5ccccc5-c5ccccc54)c4ccccc4-6)c3)nc(-c3ccc4c(c3)oc3c(-c5ccccc5)cccc34)n2)cc1] | 393 |
| 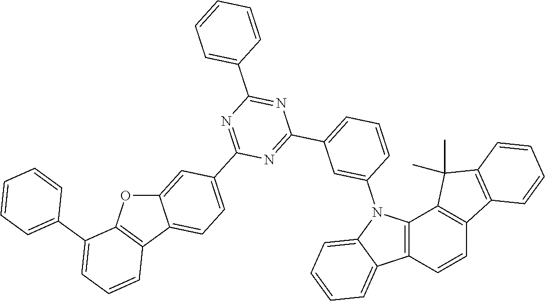 [SMILES: CC1(C)c2ccccc2-c2ccc3c4ccccc4n(-c4cccc(-c5nc(-c6ccccc6)nc(-c6ccc7c(c6)oc6c(-c8ccccc8)cccc67)n5)c4)c3c21] | 394 |
| 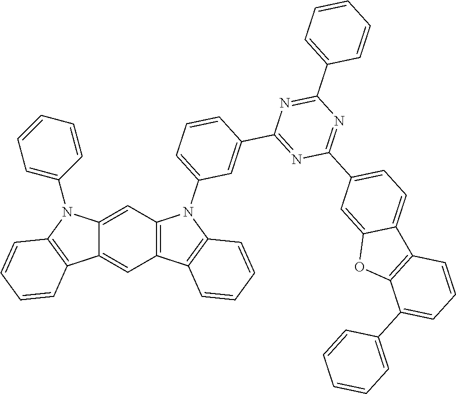 [SMILES: c1ccc(-c2nc(-c3cccc(-n4c5ccccc5c5cc6c7ccccc7n(-c7ccccc7)c6cc54)c3)nc(-c3ccc4c(c3)oc3c(-c5ccccc5)cccc34)n2)cc1] | 395 |
| 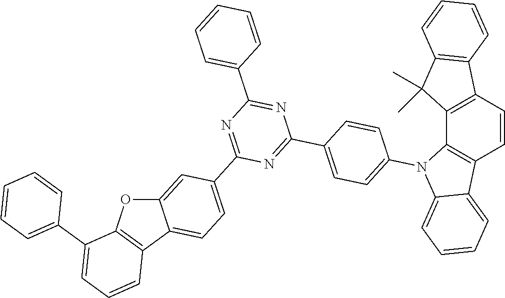 [SMILES: CC1(C)c2ccccc2-c2ccc3c4ccccc4n(-c4ccc(-c5nc(-c6ccccc6)nc(-c6ccc7c(c6)oc6c(-c8ccccc8)cccc67)n5)cc4)c3c21] | 396 |
| 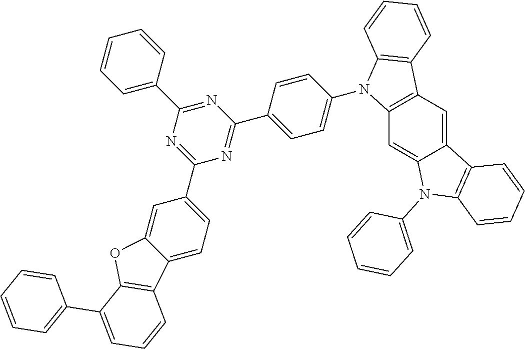 [SMILES: c1ccc(-c2nc(-c3ccc(-n4c5ccccc5c5cc6c7ccccc7n(-c7ccccc7)c6cc54)cc3)nc(-c3ccc4c(c3)oc3c(-c5ccccc5)cccc34)n2)cc1] | 397 |
| 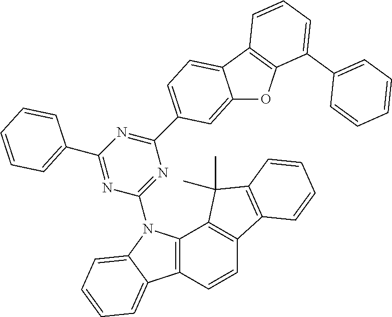 [SMILES: CC1(C)c2ccccc2-c2ccc3c4ccccc4n(-c4nc(-c5ccccc5)nc(-c5ccc6c(c5)oc5c(-c7ccccc7)cccc56)n4)c3c21] | 398 |
| 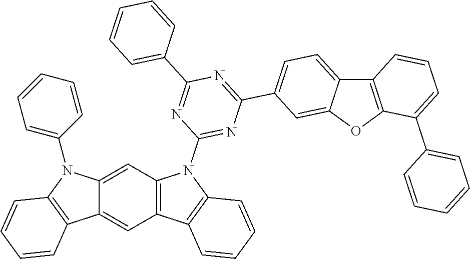 [SMILES: c1ccc(-c2nc(-c3ccc4c(c3)oc3c(-c5ccccc5)cccc34)nc(-n3c4ccccc4c4cc5c6ccccc6n(-c6ccccc6)c5cc43)n2)cc1] | 399 |
| 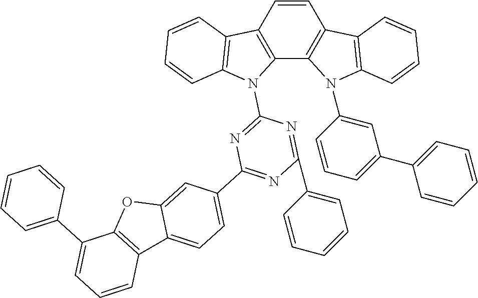 [SMILES: c1ccc(-c2cccc(-n3c4ccccc4c4ccc5c6ccccc6n(-c6nc(-c7ccccc7)nc(-c7ccc8c(c7)oc7c(-c9ccccc9)cccc78)n6)c5c43)c2)cc1] | 400 |
| 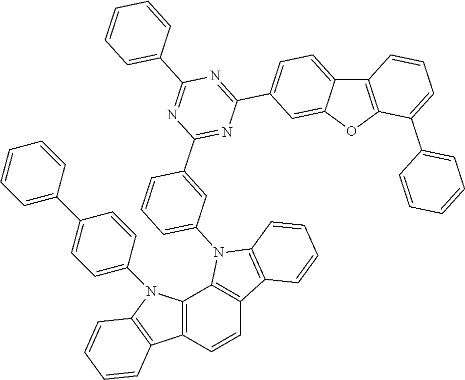 [SMILES: c1ccc(-c2ccc(-n3c4ccccc4c4ccc5c6ccccc6n(-c6cccc(-c7nc(-c8ccccc8)nc(-c8ccc9c(c8)oc8c(-c%10ccccc%10)cccc89)n7)c6)c5c43)cc2)cc1] | 401 |
| 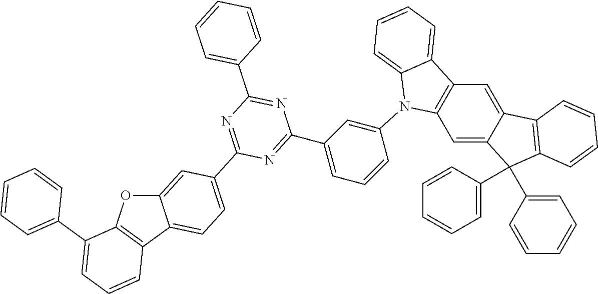 [SMILES: c1ccc(-c2nc(-c3cccc(-n4c5ccccc5c5cc6c(cc54)C(c4ccccc4)(c4ccccc4)c4ccccc4-6)c3)nc(-c3ccc4c(c3)oc3c(-c5ccccc5)cccc34)n2)cc1] | 402 |
| 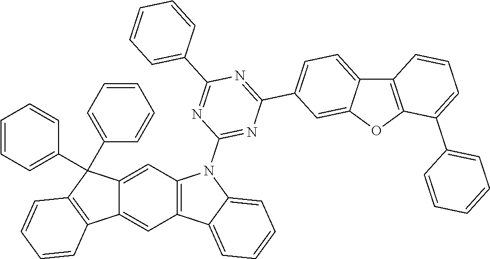 [SMILES: c1ccc(-c2nc(-c3ccc4c(c3)oc3c(-c5ccccc5)cccc34)nc(-n3c4ccccc4c4cc5c(cc43)C(c3ccccc3)(c3ccccc3)c3ccccc3-5)n2)cc1] | 403 |
| 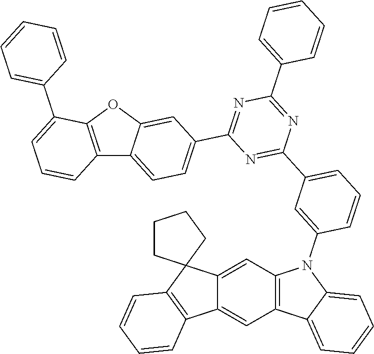 [SMILES: c1ccc(-c2nc(-c3cccc(-n4c5ccccc5c5cc6c(cc54)C4(CCCC4)c4ccccc4-6)c3)nc(-c3ccc4c(c3)oc3c(-c5ccccc5)cccc34)n2)cc1] | 404 |
| 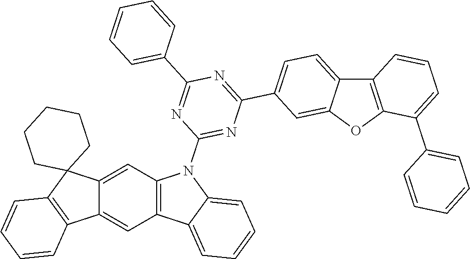 [SMILES: c1ccc(-c2nc(-c3ccc4c(c3)oc3c(-c5ccccc5)cccc34)nc(-n3c4ccccc4c4cc5c(cc43)C3(CCCCC3)c3ccccc3-5)n2)cc1] | 405 |
| 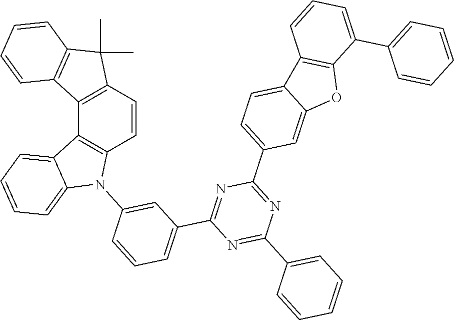 [SMILES: CC1(C)c2ccccc2-c2c1ccc1c2c2ccccc2n1-c1cccc(-c2nc(-c3ccccc3)nc(-c3ccc4c(c3)oc3c(-c5ccccc5)cccc34)n2)c1] | 406 |
| 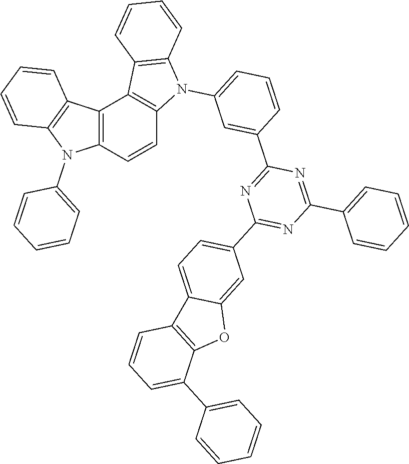 [SMILES: c1ccc(-c2nc(-c3cccc(-n4c5ccccc5c5c6c7ccccc7n(-c7ccccc7)c6ccc54)c3)nc(-c3ccc4c(c3)oc3c(-c5ccccc5)cccc34)n2)cc1] | 407 |
| 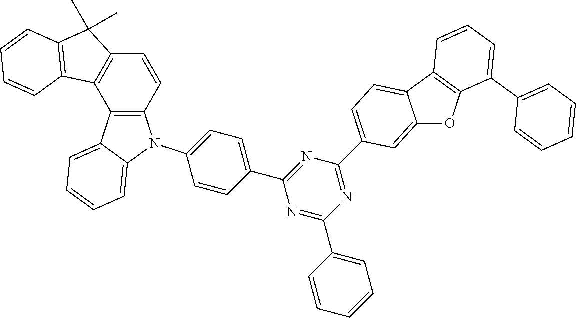 [SMILES: CC1(C)c2ccccc2-c2c1ccc1c2c2ccccc2n1-c1ccc(-c2nc(-c3ccccc3)nc(-c3ccc4c(c3)oc3c(-c5ccccc5)cccc34)n2)cc1] | 408 |
| 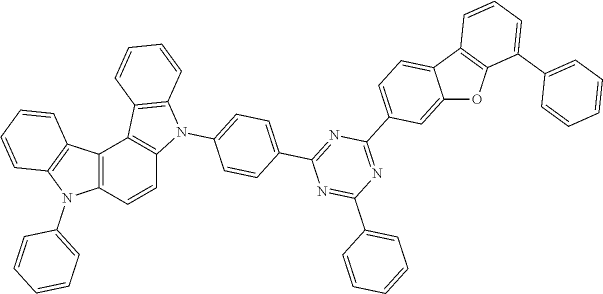 [SMILES: c1ccc(-c2nc(-c3ccc(-n4c5ccccc5c5c6c7ccccc7n(-c7ccccc7)c6ccc54)cc3)nc(-c3ccc4c(c3)oc3c(-c5ccccc5)cccc34)n2)cc1] | 409 |
| 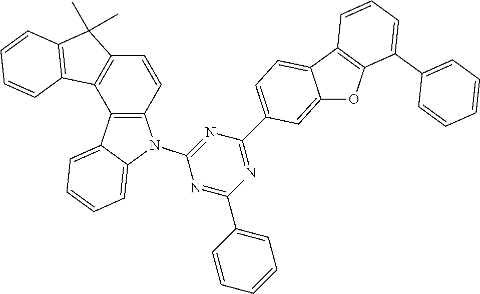 [SMILES: CC1(C)c2ccccc2-c2c1ccc1c2c2ccccc2n1-c1nc(-c2ccccc2)nc(-c2ccc3c(c2)oc2c(-c4ccccc4)cccc23)n1] | 410 |
| 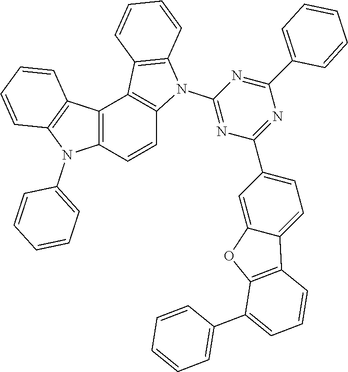 [SMILES: c1ccc(-c2nc(-c3ccc4c(c3)oc3c(-c5ccccc5)cccc34)nc(-n3c4ccccc4c4c5c6ccccc6n(-c6ccccc6)c5ccc43)n2)cc1] | 411 |
| 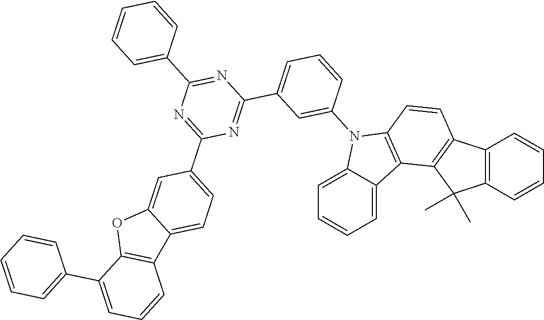 [SMILES: CC1(C)c2ccccc2-c2ccc3c(c21)c1ccccc1n3-c1cccc(-c2nc(-c3ccccc3)nc(-c3ccc4c(c3)oc3c(-c5ccccc5)cccc34)n2)c1] | 412 |
| 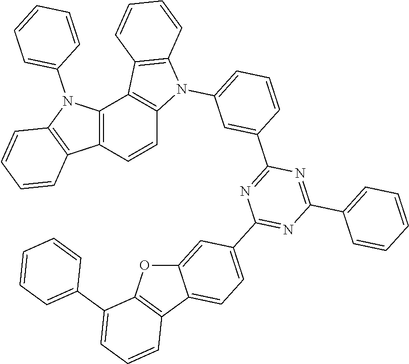 [SMILES: c1ccc(-c2nc(-c3cccc(-n4c5ccccc5c5c4ccc4c6ccccc6n(-c6ccccc6)c45)c3)nc(-c3ccc4c(c3)oc3c(-c5ccccc5)cccc34)n2)cc1] | 413 |
| 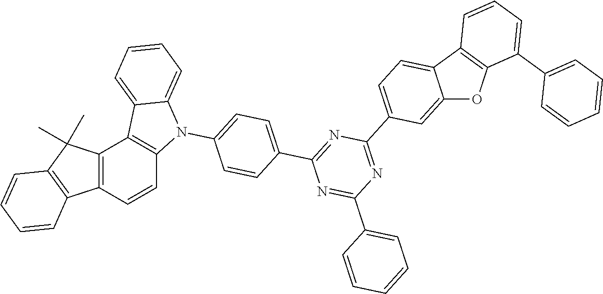 [SMILES: CC1(C)c2ccccc2-c2ccc3c(c21)c1ccccc1n3-c1ccc(-c2nc(-c3ccccc3)nc(-c3ccc4c(c3)oc3c(-c5ccccc5)cccc34)n2)cc1] | 414 |
| 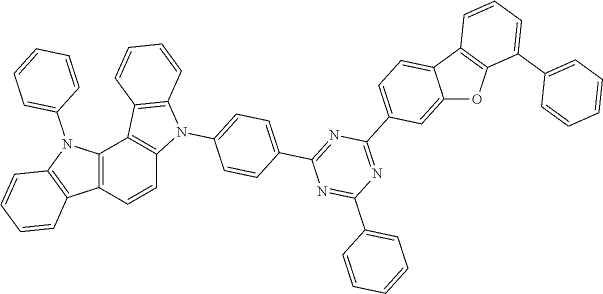 [SMILES: c1ccc(-c2nc(-c3ccc(-n4c5ccccc5c5c4ccc4c6ccccc6n(-c6ccccc6)c45)cc3)nc(-c3ccc4c(c3)oc3c(-c5ccccc5)cccc34)n2)cc1] | 415 |
| 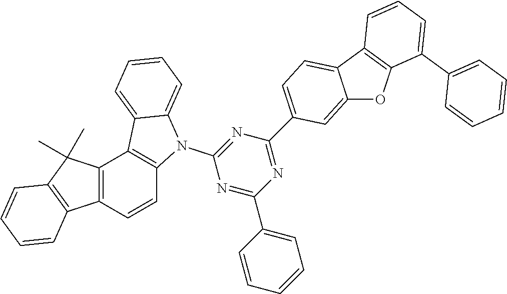 [SMILES: CC1(C)c2ccccc2-c2ccc3c(c21)c1ccccc1n3-c1nc(-c2ccccc2)nc(-c2ccc3c(c2)oc2c(-c4ccccc4)cccc23)n1] | 416 |
| 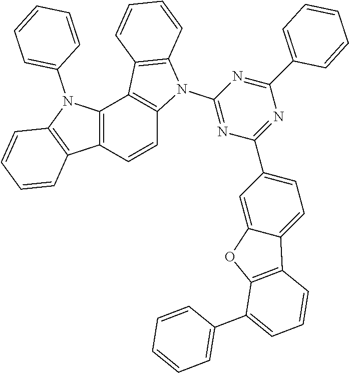 [SMILES: c1ccc(-c2nc(-c3ccc4c(c3)oc3c(-c5ccccc5)cccc34)nc(-n3c4ccccc4c4c3ccc3c5ccccc5n(-c5ccccc5)c34)n2)cc1] | 417 |
| 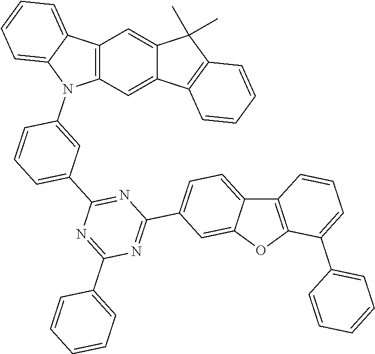 [SMILES: CC1(C)c2ccccc2-c2cc3c(cc21)c1ccccc1n3-c1cccc(-c2nc(-c3ccccc3)nc(-c3ccc4c(c3)oc3c(-c5ccccc5)cccc34)n2)c1] | 418 |
| 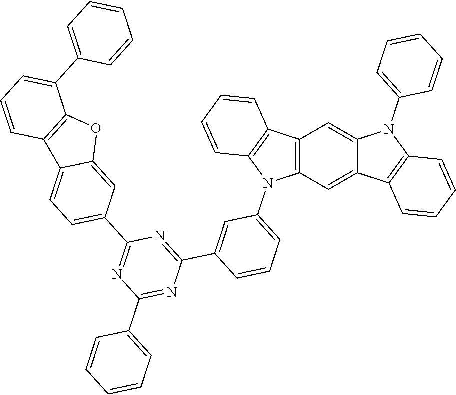 [SMILES: c1ccc(-c2nc(-c3cccc(-n4c5ccccc5c5cc6c(cc54)c4ccccc4n6-c4ccccc4)c3)nc(-c3ccc4c(c3)oc3c(-c5ccccc5)cccc34)n2)cc1] | 419 |
| 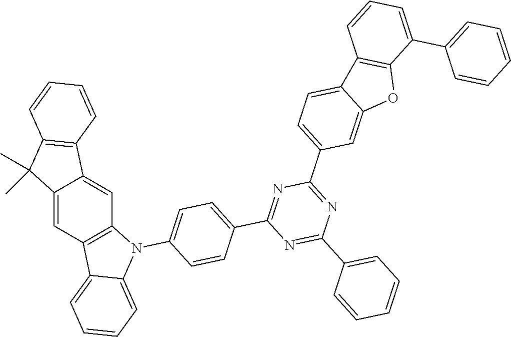 [SMILES: CC1(C)c2ccccc2-c2cc3c(cc21)c1ccccc1n3-c1ccc(-c2nc(-c3ccccc3)nc(-c3ccc4c(c3)oc3c(-c5ccccc5)cccc34)n2)cc1] | 420 |
| 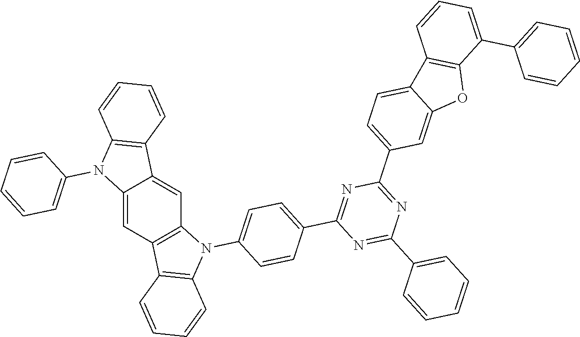 [SMILES: c1ccc(-c2nc(-c3ccc(-n4c5ccccc5c5cc6c(cc54)c4ccccc4n6-c4ccccc4)cc3)nc(-c3ccc4c(c3)oc3c(-c5ccccc5)cccc34)n2)cc1] | 421 |
| 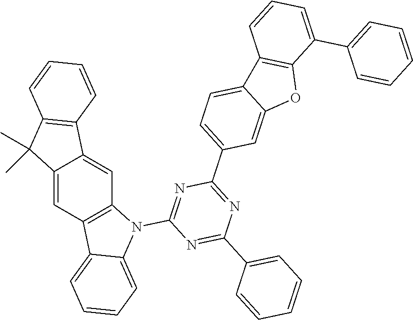 [SMILES: CC1(C)c2ccccc2-c2cc3c(cc21)c1ccccc1n3-c1nc(-c2ccccc2)nc(-c2ccc3c(c2)oc2c(-c4ccccc4)cccc23)n1] | 422 |
| 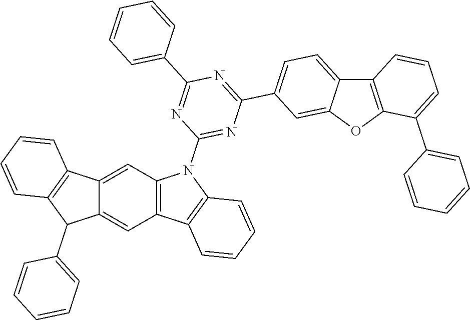 [SMILES: c1ccc(-c2nc(-c3ccc4c(c3)oc3c(-c5ccccc5)cccc34)nc(-n3c4ccccc4c4cc5c(cc43)-c3ccccc3C5c3ccccc3)n2)cc1] | 423 |
| 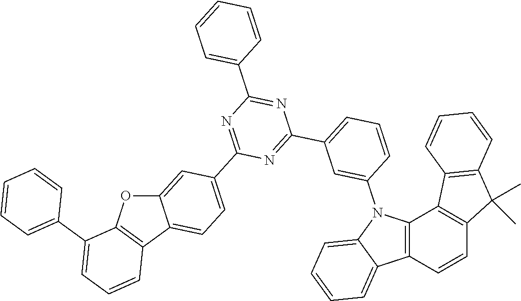 [SMILES: CC1(C)c2ccccc2-c2c1ccc1c3ccccc3n(-c3cccc(-c4nc(-c5ccccc5)nc(-c5ccc6c(c5)oc5c(-c7ccccc7)cccc56)n4)c3)c21] | 424 |
| 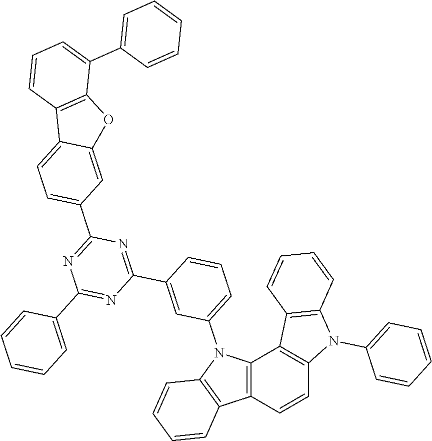 [SMILES: c1ccc(-c2nc(-c3cccc(-n4c5ccccc5c5ccc6c(c7ccccc7n6-c6ccccc6)c54)c3)nc(-c3ccc4c(c3)oc3c(-c5ccccc5)cccc34)n2)cc1] | 425 |
| 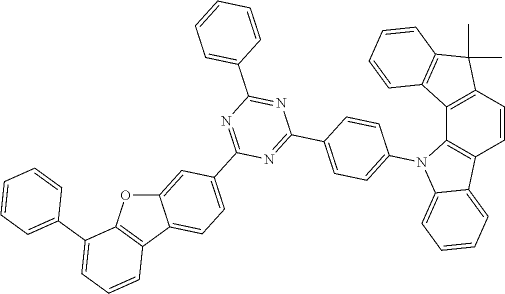 [SMILES: CC1(C)c2ccccc2-c2c1ccc1c3ccccc3n(-c3ccc(-c4nc(-c5ccccc5)nc(-c5ccc6c(c5)oc5c(-c7ccccc7)cccc56)n4)cc3)c21] | 426 |
| 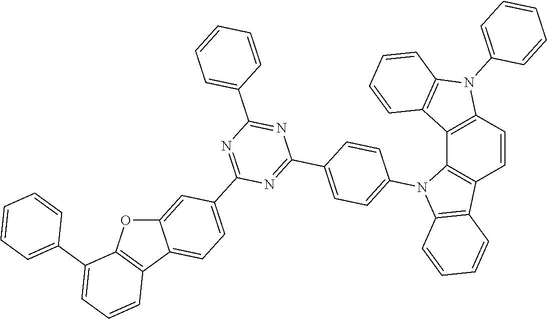 [SMILES: c1ccc(-c2nc(-c3ccc(-n4c5ccccc5c5ccc6c(c7ccccc7n6-c6ccccc6)c54)cc3)nc(-c3ccc4c(c3)oc3c(-c5ccccc5)cccc34)n2)cc1] | 427 |
| 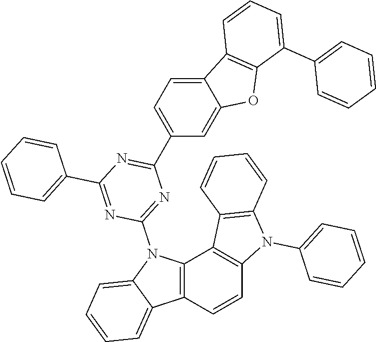 [SMILES: c1ccc(-c2nc(-c3ccc4c(c3)oc3c(-c5ccccc5)cccc34)nc(-n3c4ccccc4c4ccc5c(c6ccccc6n5-c5ccccc5)c43)n2)cc1] | 428 |
| 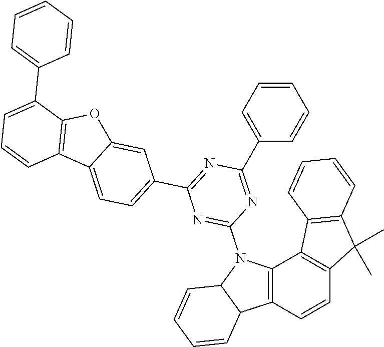 [SMILES: CC1(C)c2ccccc2-c2c1ccc1c2N(c2nc(-c3ccccc3)nc(-c3ccc4c(c3)oc3c(-c5ccccc5)cccc34)n2)C2C=CC=CC12] | 429 |
| 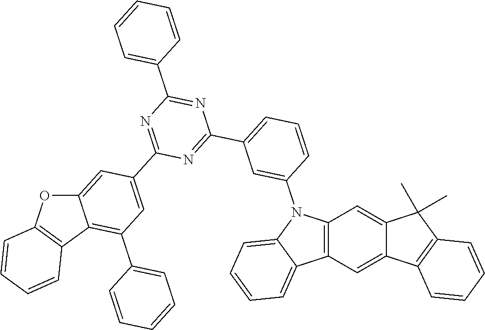 [SMILES: CC1(C)c2ccccc2-c2cc3c4ccccc4n(-c4cccc(-c5nc(-c6ccccc6)nc(-c6cc(-c7ccccc7)c7c(c6)oc6ccccc67)n5)c4)c3cc21] | 430 |
| 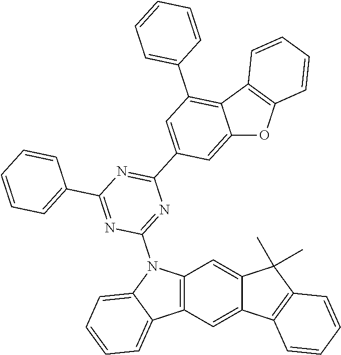 [SMILES: CC1(C)c2ccccc2-c2cc3c4ccccc4n(-c4nc(-c5ccccc5)nc(-c5cc(-c6ccccc6)c6c(c5)oc5ccccc56)n4)c3cc21] | 431 |
| 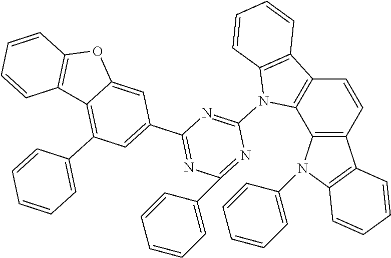 [SMILES: c1ccc(-c2nc(-c3cc(-c4ccccc4)c4c(c3)oc3ccccc34)nc(-n3c4ccccc4c4ccc5c6ccccc6n(-c6ccccc6)c5c43)n2)cc1] | 432 |
| 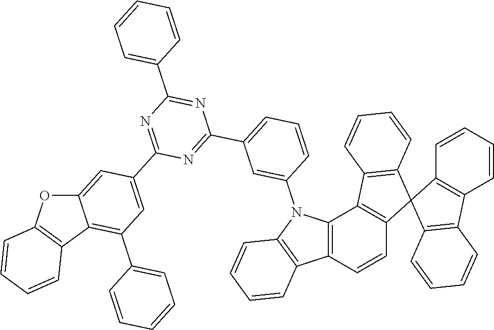 [SMILES: c1ccc(-c2nc(-c3cccc(-n4c5ccccc5c5ccc6c(c54)-c4ccccc4C64c5ccccc5-c5ccccc54)c3)nc(-c3cc(-c4ccccc4)c4c(c3)oc3ccccc34)n2)cc1] | 433 |
| 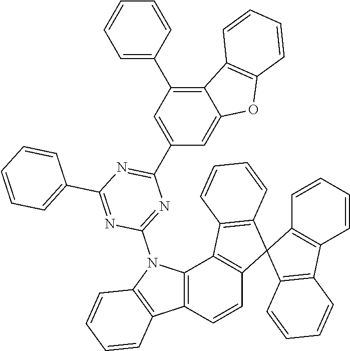 [SMILES: c1ccc(-c2nc(-c3cc(-c4ccccc4)c4c(c3)oc3ccccc34)nc(-n3c4ccccc4c4ccc5c(c43)-c3ccccc3C53c4ccccc4-c4ccccc43)n2)cc1] | 434 |
| 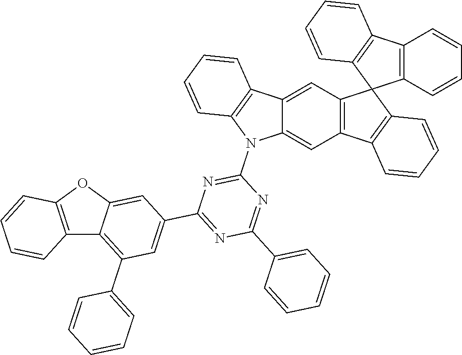 [SMILES: c1ccc(-c2nc(-c3cc(-c4ccccc4)c4c(c3)oc3ccccc34)nc(-n3c4ccccc4c4cc5c(cc43)-c3ccccc3C53c4ccccc4-c4ccccc43)n2)cc1] | 435 |
| 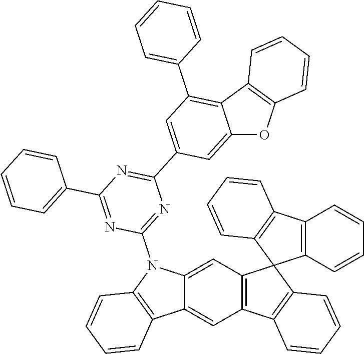 [SMILES: c1ccc(-c2nc(-c3cc(-c4ccccc4)c4c(c3)oc3ccccc34)nc(-n3c4ccccc4c4cc5c(cc43)C3(c4ccccc4-c4ccccc43)c3ccccc3-5)n2)cc1] | 436 |
| 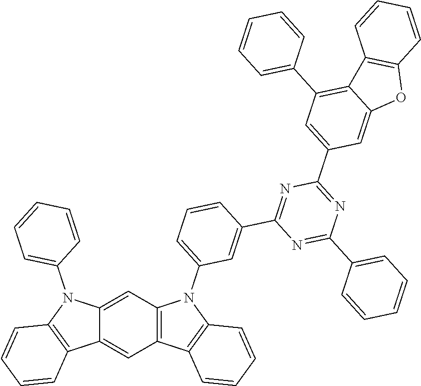 [SMILES: c1ccc(-c2nc(-c3cccc(-n4c5ccccc5c5cc6c7ccccc7n(-c7ccccc7)c6cc54)c3)nc(-c3cc(-c4ccccc4)c4c(c3)oc3ccccc34)n2)cc1] | 437 |
| 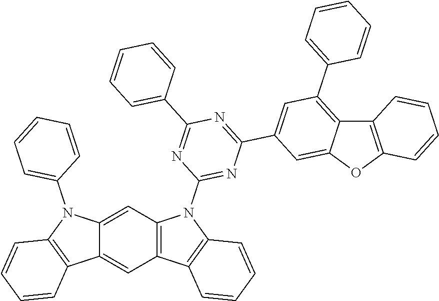 [SMILES: c1ccc(-c2nc(-c3cc(-c4ccccc4)c4c(c3)oc3ccccc34)nc(-n3c4ccccc4c4cc5c6ccccc6n(-c6ccccc6)c5cc43)n2)cc1] | 438 |
| 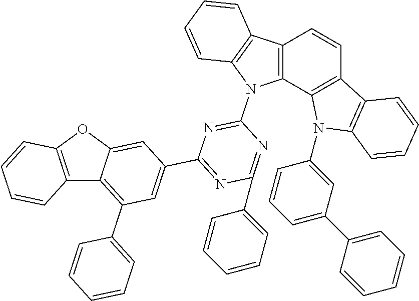 [SMILES: c1ccc(-c2cccc(-n3c4ccccc4c4ccc5c6ccccc6n(-c6nc(-c7ccccc7)nc(-c7cc(-c8ccccc8)c8c(c7)oc7ccccc78)n6)c5c43)c2)cc1] | 439 |
| 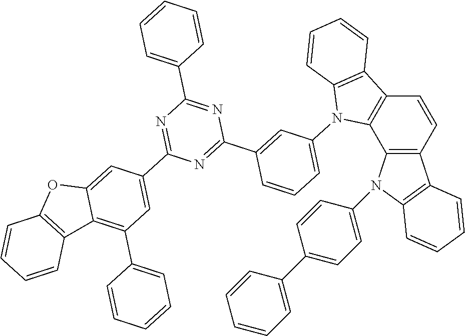 [SMILES: c1ccc(-c2ccc(-n3c4ccccc4c4ccc5c6ccccc6n(-c6cccc(-c7nc(-c8ccccc8)nc(-c8cc(-c9ccccc9)c9c(c8)oc8ccccc89)n7)c6)c5c43)cc2)cc1] | 440 |
| 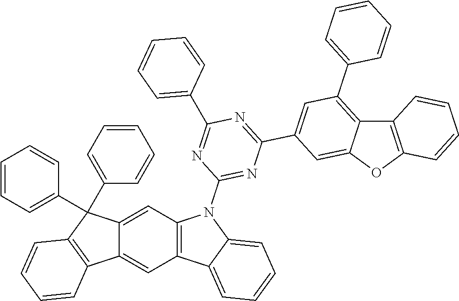 [SMILES: c1ccc(-c2nc(-c3cc(-c4ccccc4)c4c(c3)oc3ccccc34)nc(-n3c4ccccc4c4cc5c(cc43)C(c3ccccc3)(c3ccccc3)c3ccccc3-5)n2)cc1] | 441 |
| 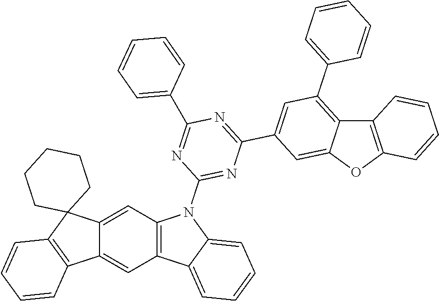 [SMILES: c1ccc(-c2nc(-c3cc(-c4ccccc4)c4c(c3)oc3ccccc34)nc(-n3c4ccccc4c4cc5c(cc43)C3(CCCCC3)c3ccccc3-5)n2)cc1] | 442 |
| 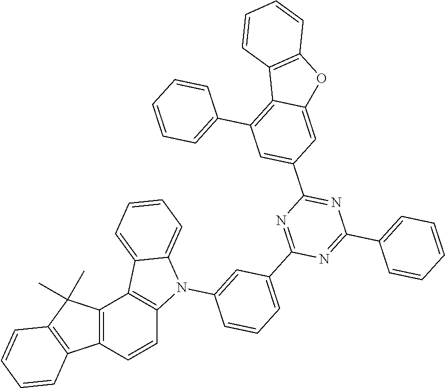 [SMILES: CC1(C)c2ccccc2-c2ccc3c(c21)c1ccccc1n3-c1cccc(-c2nc(-c3ccccc3)nc(-c3cc(-c4ccccc4)c4c(c3)oc3ccccc34)n2)c1] | 443 |
| 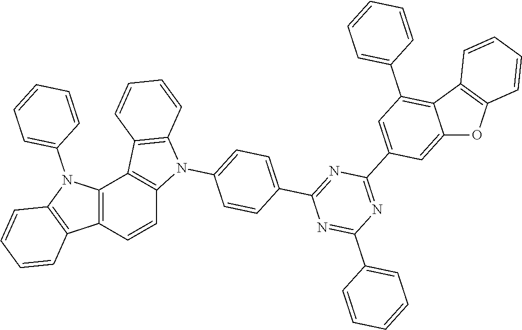 [SMILES: c1ccc(-c2nc(-c3ccc(-n4c5ccccc5c5c4ccc4c6ccccc6n(-c6ccccc6)c45)cc3)nc(-c3cc(-c4ccccc4)c4c(c3)oc3ccccc34)n2)cc1] | 444 |
| 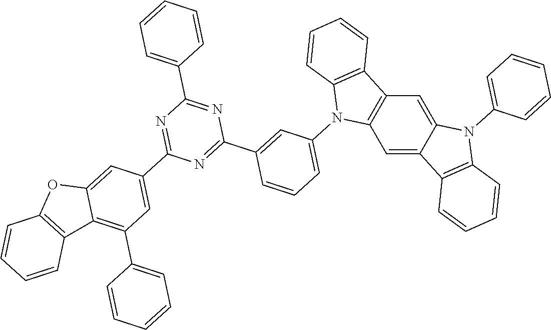 [SMILES: c1ccc(-c2nc(-c3cccc(-n4c5ccccc5c5cc6c(cc54)c4ccccc4n6-c4ccccc4)c3)nc(-c3cc(-c4ccccc4)c4c(c3)oc3ccccc34)n2)cc1] | 445 |
| 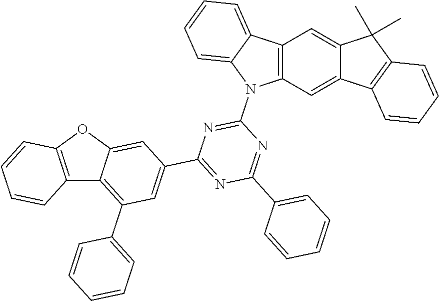 [SMILES: CC1(C)c2ccccc2-c2cc3c(cc21)c1ccccc1n3-c1nc(-c2ccccc2)nc(-c2cc(-c3ccccc3)c3c(c2)oc2ccccc23)n1] | 446 |
| 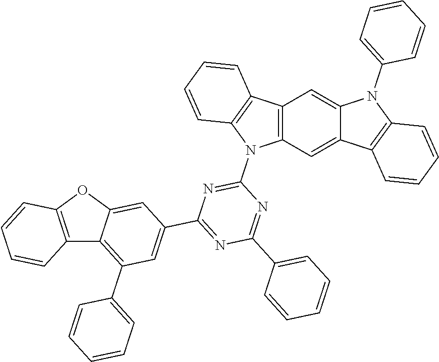 [SMILES: c1ccc(-c2nc(-c3cc(-c4ccccc4)c4c(c3)oc3ccccc34)nc(-n3c4ccccc4c4cc5c(cc43)c3ccccc3n5-c3ccccc3)n2)cc1] | 447 |
| 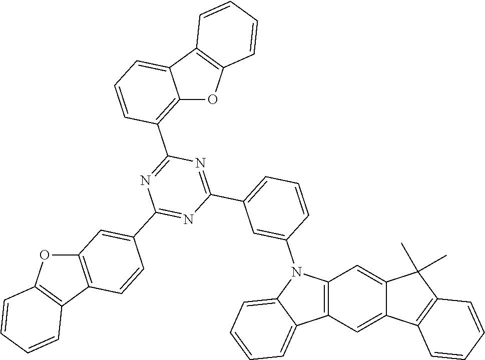 [SMILES: CC1(C)c2ccccc2-c2cc3c4ccccc4n(-c4cccc(-c5nc(-c6ccc7c(c6)oc6ccccc67)nc(-c6cccc7c6oc6ccccc67)n5)c4)c3cc21] | 448 |
| 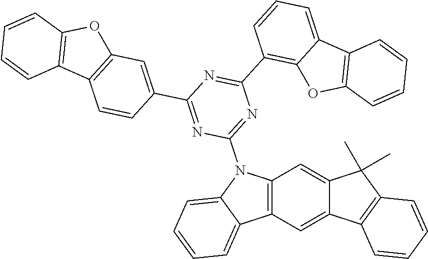 [SMILES: CC1(C)c2ccccc2-c2cc3c4ccccc4n(-c4nc(-c5ccc6c(c5)oc5ccccc56)nc(-c5cccc6c5oc5ccccc56)n4)c3cc21] | 449 |
| 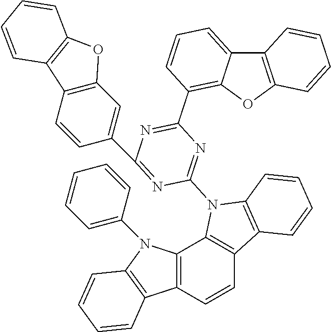 [SMILES: c1ccc(-n2c3ccccc3c3ccc4c5ccccc5n(-c5nc(-c6ccc7c(c6)oc6ccccc67)nc(-c6cccc7c6oc6ccccc67)n5)c4c32)cc1] | 450 |
| 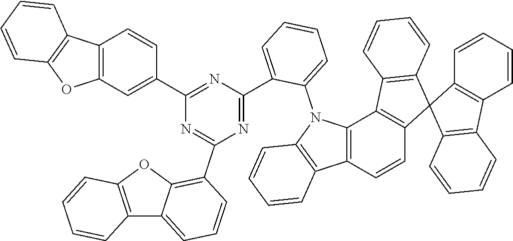 [SMILES: c1ccc(-n2c3ccccc3c3ccc4c(c32)-c2ccccc2C42c3ccccc3-c3ccccc32)c(-c2nc(-c3ccc4c(c3)oc3ccccc34)nc(-c3cccc4c3oc3ccccc34)n2)c1] | 451 |
| 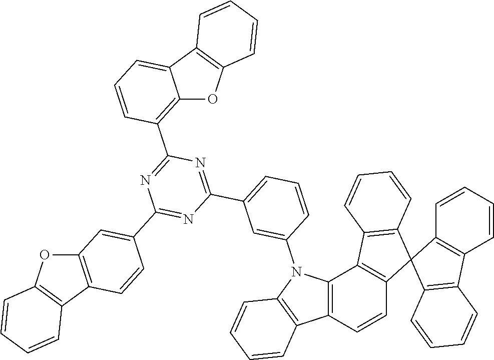 [SMILES: c1cc(-c2nc(-c3ccc4c(c3)oc3ccccc34)nc(-c3cccc4c3oc3ccccc34)n2)cc(-n2c3ccccc3c3ccc4c(c32)-c2ccccc2C42c3ccccc3-c3ccccc32)c1] | 452 |
| 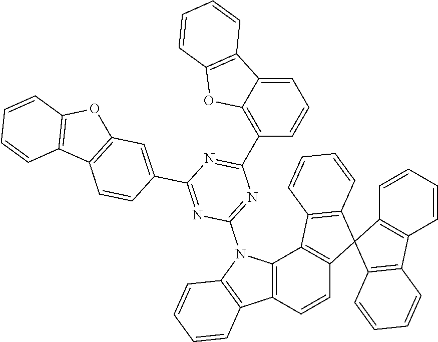 [SMILES: c1ccc2c(c1)-c1ccccc1C21c2ccccc2-c2c1ccc1c3ccccc3n(-c3nc(-c4ccc5c(c4)oc4ccccc45)nc(-c4cccc5c4oc4ccccc45)n3)c21] | 453 |
| 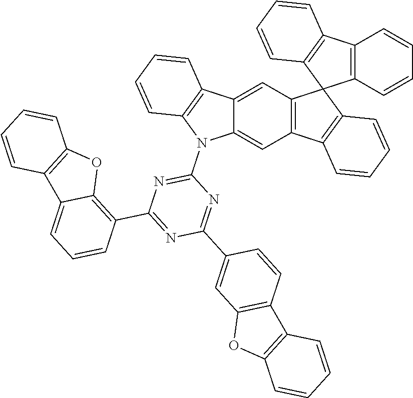 [SMILES: c1ccc2c(c1)-c1ccccc1C21c2ccccc2-c2cc3c(cc21)c1ccccc1n3-c1nc(-c2ccc3c(c2)oc2ccccc23)nc(-c2cccc3c2oc2ccccc23)n1] | 454 |
| 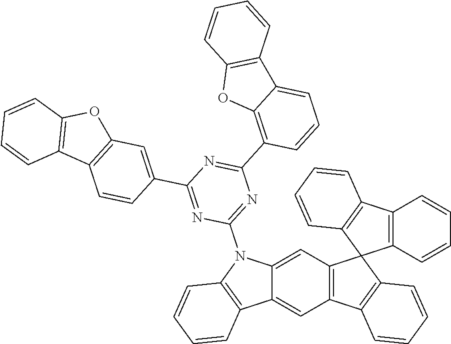 [SMILES: c1ccc2c(c1)-c1ccccc1C21c2ccccc2-c2cc3c4ccccc4n(-c4nc(-c5ccc6c(c5)oc5ccccc56)nc(-c5cccc6c5oc5ccccc56)n4)c3cc21] | 455 |
| 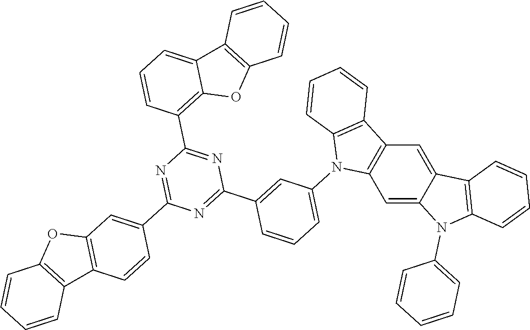 [SMILES: c1ccc(-n2c3ccccc3c3cc4c5ccccc5n(-c5cccc(-c6nc(-c7ccc8c(c7)oc7ccccc78)nc(-c7cccc8c7oc7ccccc78)n6)c5)c4cc32)cc1] | 456 |
| 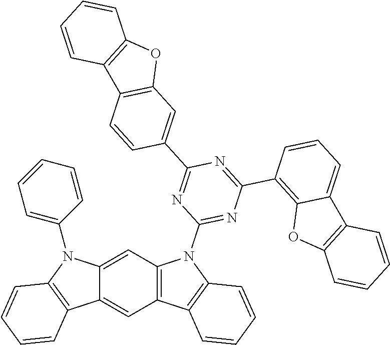 [SMILES: c1ccc(-n2c3ccccc3c3cc4c5ccccc5n(-c5nc(-c6ccc7c(c6)oc6ccccc67)nc(-c6cccc7c6oc6ccccc67)n5)c4cc32)cc1] | 457 |
| 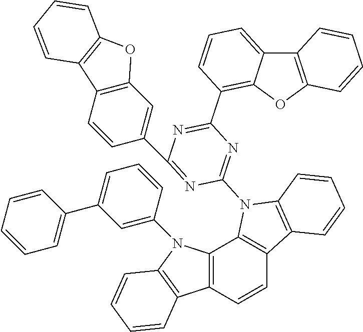 [SMILES: c1ccc(-c2cccc(-n3c4ccccc4c4ccc5c6ccccc6n(-c6nc(-c7ccc8c(c7)oc7ccccc78)nc(-c7cccc8c7oc7ccccc78)n6)c5c43)c2)cc1] | 458 |
| 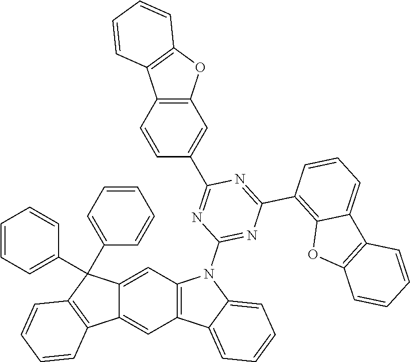 [SMILES: c1ccc(C2(c3ccccc3)c3ccccc3-c3cc4c5ccccc5n(-c5nc(-c6ccc7c(c6)oc6ccccc67)nc(-c6cccc7c6oc6ccccc67)n5)c4cc32)cc1] | 459 |
| 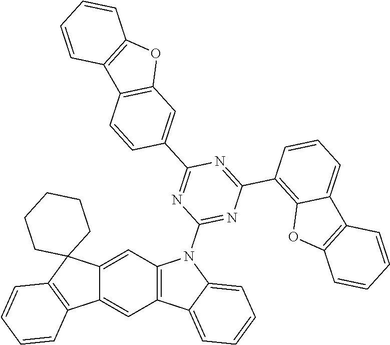 [SMILES: c1ccc2c(c1)-c1cc3c4ccccc4n(-c4nc(-c5ccc6c(c5)oc5ccccc56)nc(-c5cccc6c5oc5ccccc56)n4)c3cc1C21CCCCC1] | 460 |
| 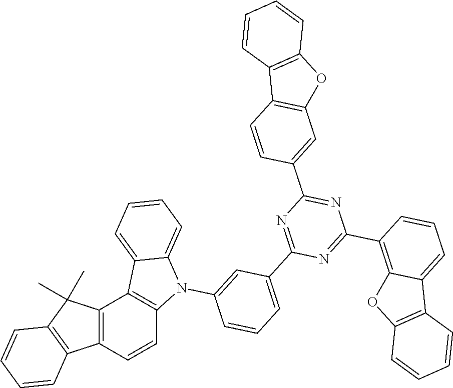 [SMILES: CC1(C)c2ccccc2-c2ccc3c(c21)c1ccccc1n3-c1cccc(-c2nc(-c3ccc4c(c3)oc3ccccc34)nc(-c3cccc4c3oc3ccccc34)n2)c1] | 461 |
| 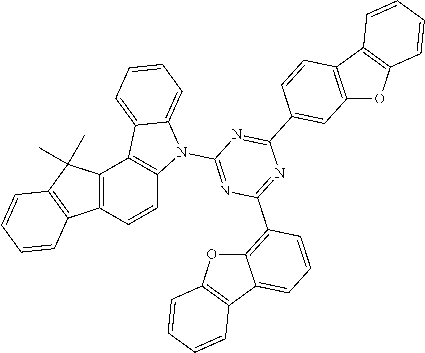 [SMILES: CC1(C)c2ccccc2-c2ccc3c(c21)c1ccccc1n3-c1nc(-c2ccc3c(c2)oc2ccccc23)nc(-c2cccc3c2oc2ccccc23)n1] | 462 |
| 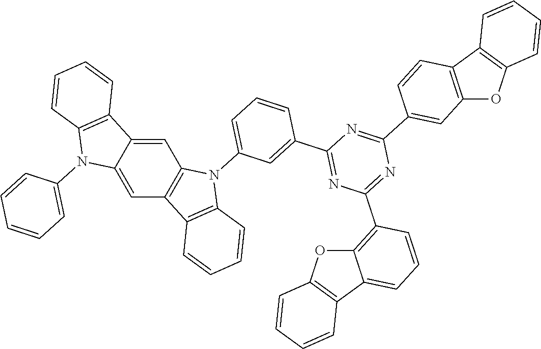 [SMILES: c1ccc(-n2c3ccccc3c3cc4c(cc32)c2ccccc2n4-c2cccc(-c3nc(-c4ccc5c(c4)oc4ccccc45)nc(-c4cccc5c4oc4ccccc45)n3)c2)cc1] | 463 |
| 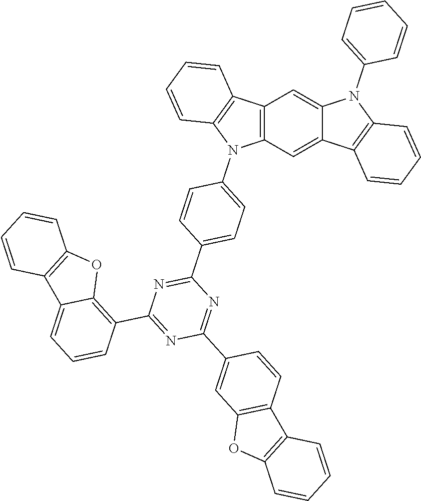 [SMILES: c1ccc(-n2c3ccccc3c3cc4c(cc32)c2ccccc2n4-c2ccc(-c3nc(-c4ccc5c(c4)oc4ccccc45)nc(-c4cccc5c4oc4ccccc45)n3)cc2)cc1] | 464 |
| 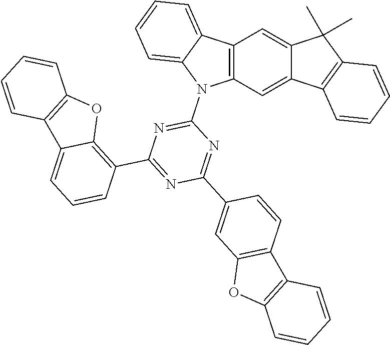 [SMILES: CC1(C)c2ccccc2-c2cc3c(cc21)c1ccccc1n3-c1nc(-c2ccc3c(c2)oc2ccccc23)nc(-c2cccc3c2oc2ccccc23)n1] | 465 |
| 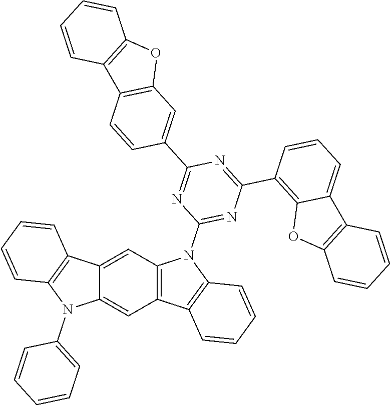 [SMILES: c1ccc(-n2c3ccccc3c3cc4c(cc32)c2ccccc2n4-c2nc(-c3ccc4c(c3)oc3ccccc34)nc(-c3cccc4c3oc3ccccc34)n2)cc1] | 466 |
| 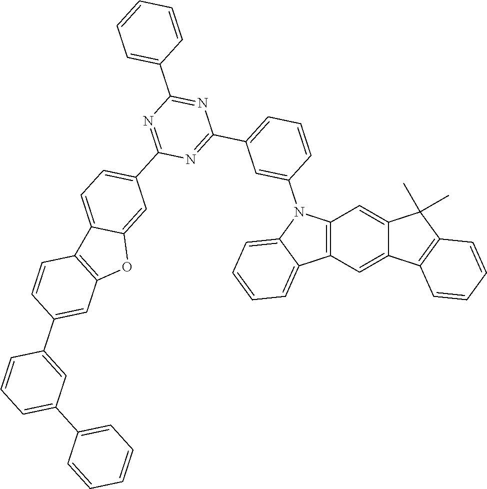 [SMILES: CC1(C)c2ccccc2-c2cc3c4ccccc4n(-c4cccc(-c5nc(-c6ccccc6)nc(-c6ccc7c(c6)oc6cc(-c8cccc(-c9ccccc9)c8)ccc67)n5)c4)c3cc21] | 467 |
| 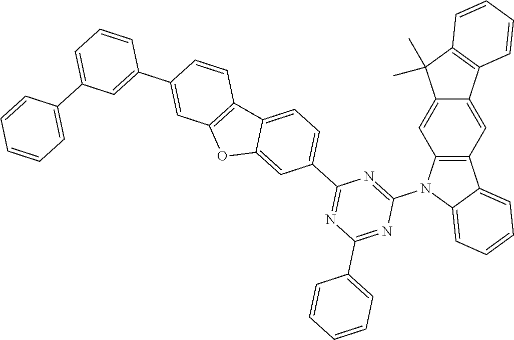 [SMILES: CC1(C)c2ccccc2-c2cc3c4ccccc4n(-c4nc(-c5ccccc5)nc(-c5ccc6c(c5)oc5cc(-c7cccc(-c8ccccc8)c7)ccc56)n4)c3cc21] | 468 |
| 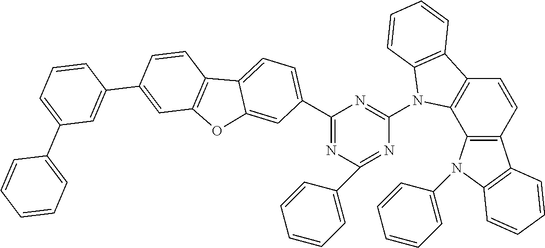 [SMILES: c1ccc(-c2cccc(-c3ccc4c(c3)oc3cc(-c5nc(-c6ccccc6)nc(-n6c7ccccc7c7ccc8c9ccccc9n(-c9ccccc9)c8c76)n5)ccc34)c2)cc1] | 469 |
| 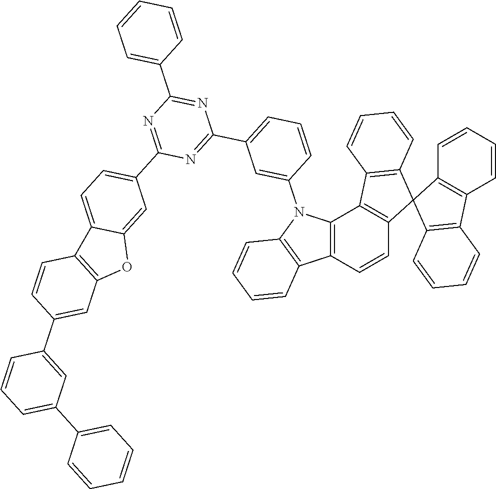 [SMILES: c1ccc(-c2cccc(-c3ccc4c(c3)oc3cc(-c5nc(-c6ccccc6)nc(-c6cccc(-n7c8ccccc8c8ccc9c(c87)-c7ccccc7C97c8ccccc8-c8ccccc87)c6)n5)ccc34)c2)cc1] | 470 |
| 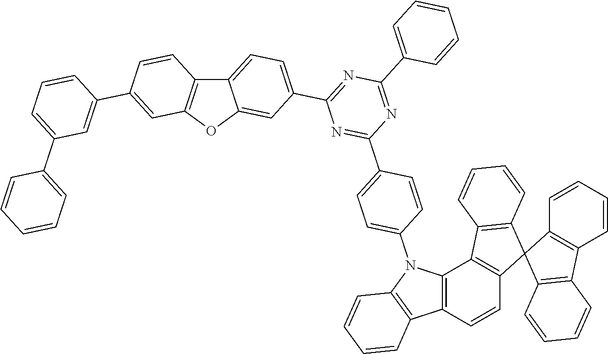 [SMILES: c1ccc(-c2cccc(-c3ccc4c(c3)oc3cc(-c5nc(-c6ccccc6)nc(-c6ccc(-n7c8ccccc8c8ccc9c(c87)-c7ccccc7C97c8ccccc8-c8ccccc87)cc6)n5)ccc34)c2)cc1] | 471 |
| 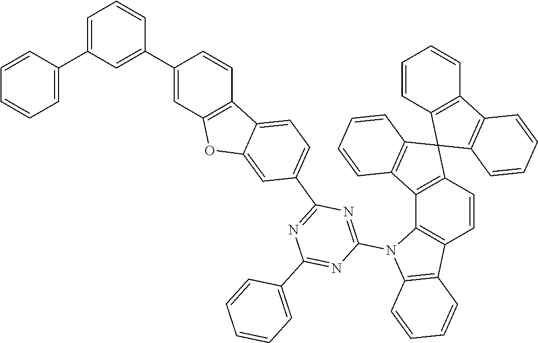 [SMILES: c1ccc(-c2cccc(-c3ccc4c(c3)oc3cc(-c5nc(-c6ccccc6)nc(-n6c7ccccc7c7ccc8c(c76)-c6ccccc6C86c7ccccc7-c7ccccc76)n5)ccc34)c2)cc1] | 472 |
| 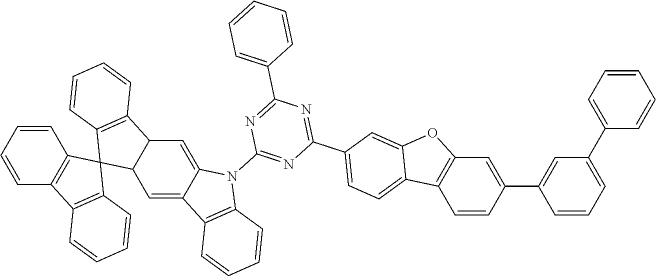 [SMILES: C1=c2c(n(-c3nc(-c4ccccc4)nc(-c4ccc5c(c4)oc4cc(-c6cccc(-c7ccccc7)c6)ccc45)n3)c3ccccc23)=CC2c3ccccc3C3(c4ccccc4-c4ccccc43)C12] | 473 |
| 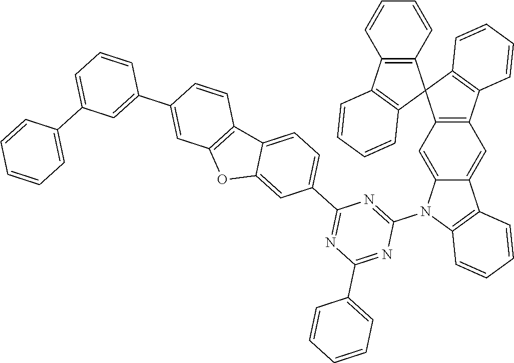 [SMILES: c1ccc(-c2cccc(-c3ccc4c(c3)oc3cc(-c5nc(-c6ccccc6)nc(-n6c7ccccc7c7cc8c(cc76)C6(c7ccccc7-c7ccccc76)c6ccccc6-8)n5)ccc34)c2)cc1] | 474 |
| 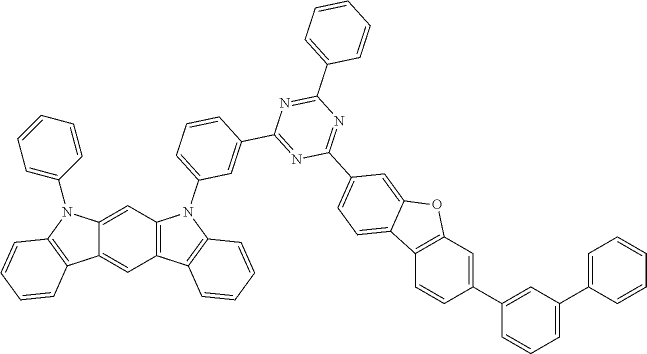 [SMILES: c1ccc(-c2cccc(-c3ccc4c(c3)oc3cc(-c5nc(-c6ccccc6)nc(-c6cccc(-n7c8ccccc8c8cc9c%10ccccc%10n(-c%10ccccc%10)c9cc87)c6)n5)ccc34)c2)cc1] | 475 |
| 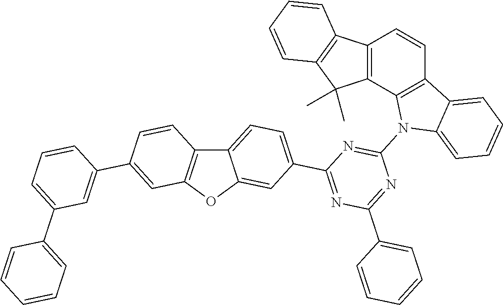 [SMILES: CC1(C)c2ccccc2-c2ccc3c4ccccc4n(-c4nc(-c5ccccc5)nc(-c5ccc6c(c5)oc5cc(-c7cccc(-c8ccccc8)c7)ccc56)n4)c3c21] | 476 |
| 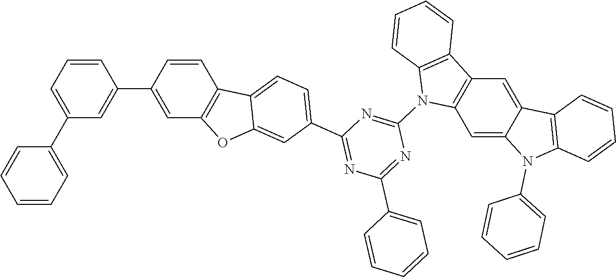 [SMILES: c1ccc(-c2cccc(-c3ccc4c(c3)oc3cc(-c5nc(-c6ccccc6)nc(-n6c7ccccc7c7cc8c9ccccc9n(-c9ccccc9)c8cc76)n5)ccc34)c2)cc1] | 477 |
| 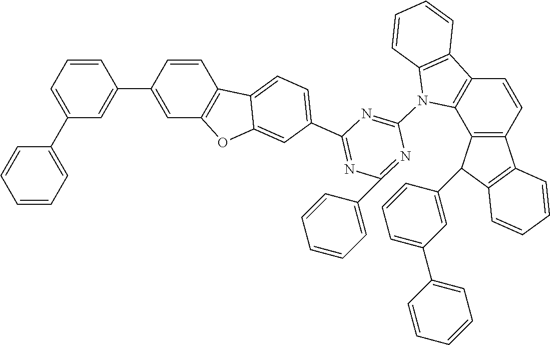 [SMILES: c1ccc(-c2cccc(-c3ccc4c(c3)oc3cc(-c5nc(-c6ccccc6)nc(-n6c7ccccc7c7ccc8c(c76)C(c6cccc(-c7ccccc7)c6)c6ccccc6-8)n5)ccc34)c2)cc1] | 478 |
| 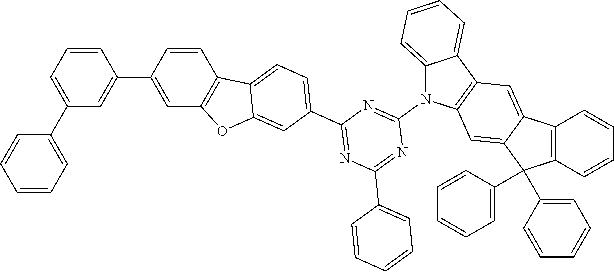 [SMILES: c1ccc(-c2cccc(-c3ccc4c(c3)oc3cc(-c5nc(-c6ccccc6)nc(-n6c7ccccc7c7cc8c(cc76)C(c6ccccc6)(c6ccccc6)c6ccccc6-8)n5)ccc34)c2)cc1] | 479 |
| 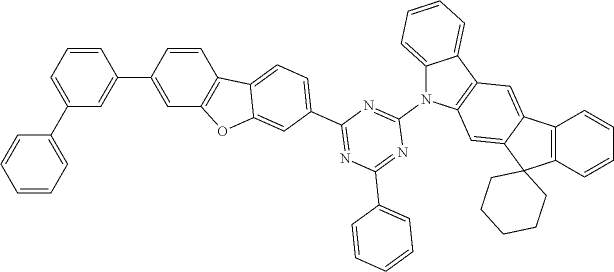 [SMILES: c1ccc(-c2cccc(-c3ccc4c(c3)oc3cc(-c5nc(-c6ccccc6)nc(-n6c7ccccc7c7cc8c(cc76)C6(CCCCC6)c6ccccc6-8)n5)ccc34)c2)cc1] | 480 |
| 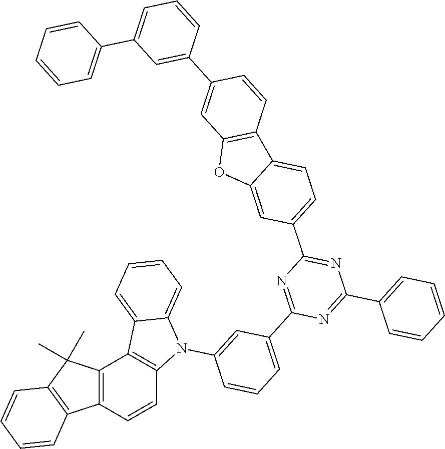 [SMILES: CC1(C)c2ccccc2-c2ccc3c(c21)c1ccccc1n3-c1cccc(-c2nc(-c3ccccc3)nc(-c3ccc4c(c3)oc3cc(-c5cccc(-c6ccccc6)c5)ccc34)n2)c1] | 481 |
| 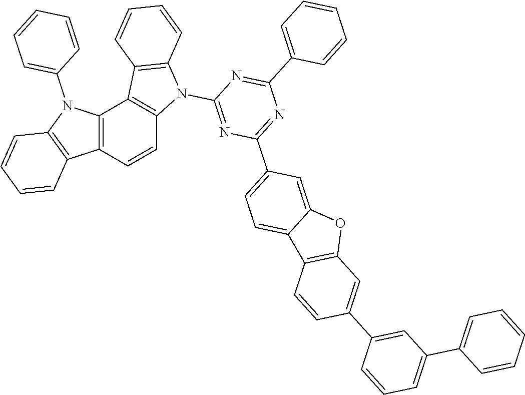 [SMILES: c1ccc(-c2cccc(-c3ccc4c(c3)oc3cc(-c5nc(-c6ccccc6)nc(-n6c7ccccc7c7c6ccc6c8ccccc8n(-c8ccccc8)c67)n5)ccc34)c2)cc1] | 482 |
| 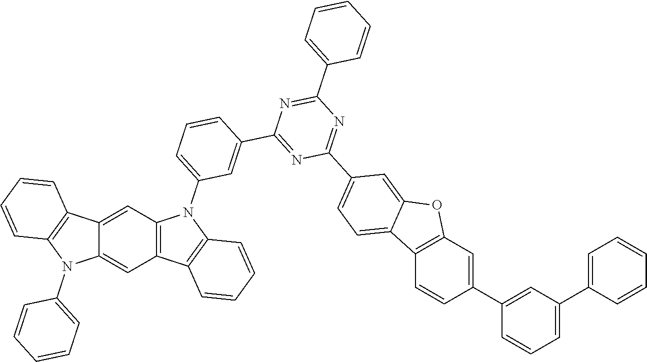 [SMILES: c1ccc(-c2cccc(-c3ccc4c(c3)oc3cc(-c5nc(-c6ccccc6)nc(-c6cccc(-n7c8ccccc8c8cc9c(cc87)c7ccccc7n9-c7ccccc7)c6)n5)ccc34)c2)cc1] | 483 |
| 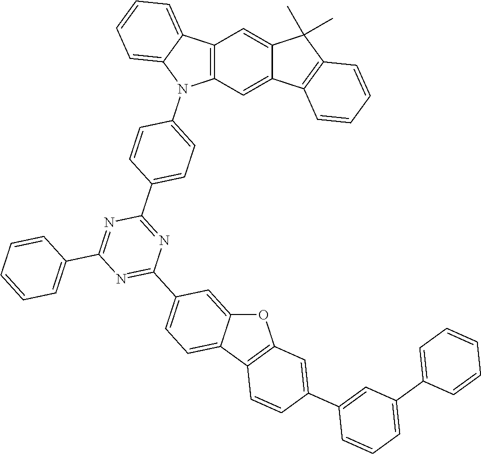 [SMILES: CC1(C)c2ccccc2-c2cc3c(cc21)c1ccccc1n3-c1ccc(-c2nc(-c3ccccc3)nc(-c3ccc4c(c3)oc3cc(-c5cccc(-c6ccccc6)c5)ccc34)n2)cc1] | 484 |
| 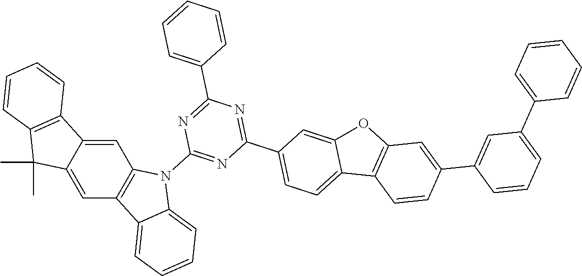 [SMILES: CC1(C)c2ccccc2-c2cc3c(cc21)c1ccccc1n3-c1nc(-c2ccccc2)nc(-c2ccc3c(c2)oc2cc(-c4cccc(-c5ccccc5)c4)ccc23)n1] | 485 |
| 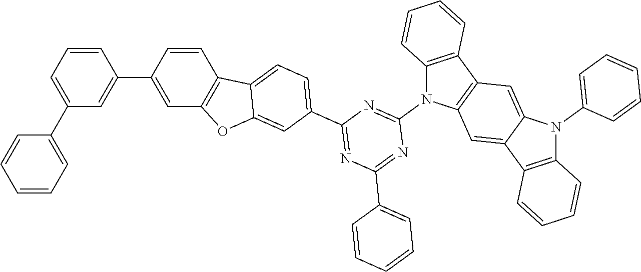 [SMILES: c1ccc(-c2cccc(-c3ccc4c(c3)oc3cc(-c5nc(-c6ccccc6)nc(-n6c7ccccc7c7cc8c(cc76)c6ccccc6n8-c6ccccc6)n5)ccc34)c2)cc1] | 486 |
| 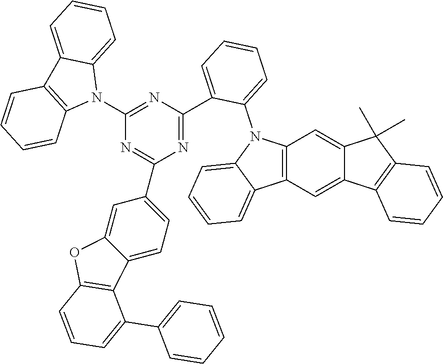 [SMILES: CC1(C)c2ccccc2-c2cc3c4ccccc4n(-c4ccccc4-c4nc(-c5ccc6c(c5)oc5cccc(-c7ccccc7)c56)nc(-n5c6ccccc6c6ccccc65)n4)c3cc21] | 487 |
| 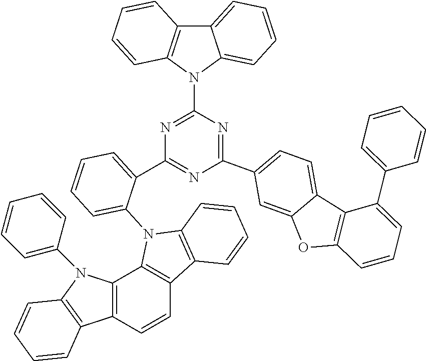 [SMILES: c1ccc(-c2cccc3oc4cc(-c5nc(-c6ccccc6-n6c7ccccc7c7ccc8c9ccccc9n(-c9ccccc9)c8c76)nc(-n6c7ccccc7c7ccccc76)n5)ccc4c23)cc1] | 488 |
| 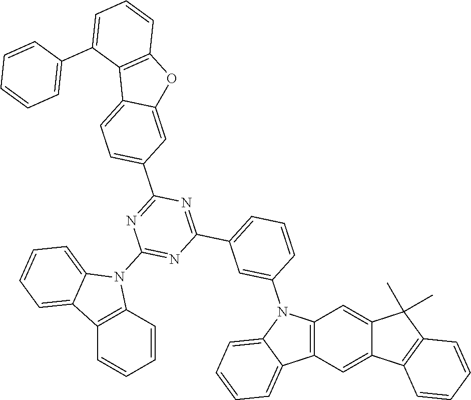 [SMILES: CC1(C)c2ccccc2-c2cc3c4ccccc4n(-c4cccc(-c5nc(-c6ccc7c(c6)oc6cccc(-c8ccccc8)c67)nc(-n6c7ccccc7c7ccccc76)n5)c4)c3cc21] | 489 |
| 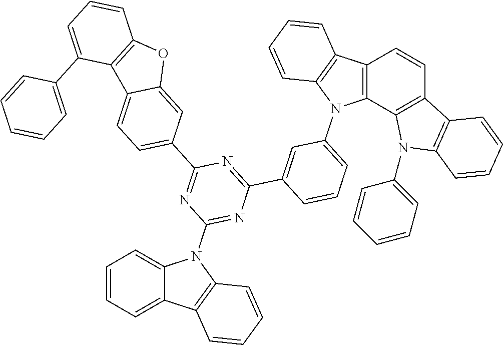 [SMILES: c1ccc(-c2cccc3oc4cc(-c5nc(-c6cccc(-n7c8ccccc8c8ccc9c%10ccccc%10n(-c%10ccccc%10)c9c87)c6)nc(-n6c7ccccc7c7ccccc76)n5)ccc4c23)cc1] | 490 |
| 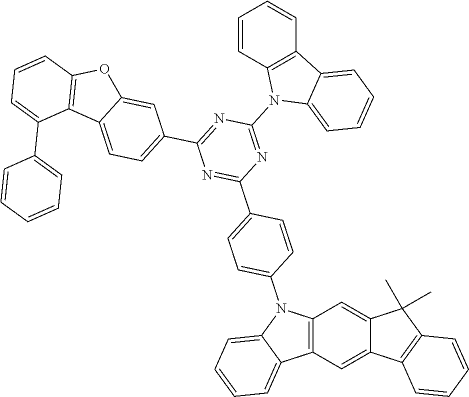 [SMILES: CC1(C)c2ccccc2-c2cc3c4ccccc4n(-c4ccc(-c5nc(-c6ccc7c(c6)oc6cccc(-c8ccccc8)c67)nc(-n6c7ccccc7c7ccccc76)n5)cc4)c3cc21] | 491 |
| 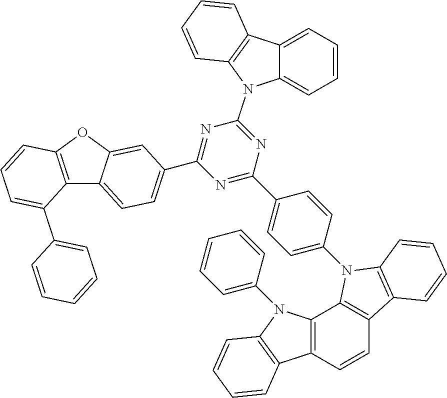 [SMILES: c1ccc(-c2cccc3oc4cc(-c5nc(-c6ccc(-n7c8ccccc8c8ccc9c%10ccccc%10n(-c%10ccccc%10)c9c87)cc6)nc(-n6c7ccccc7c7ccccc76)n5)ccc4c23)cc1] | 492 |
| 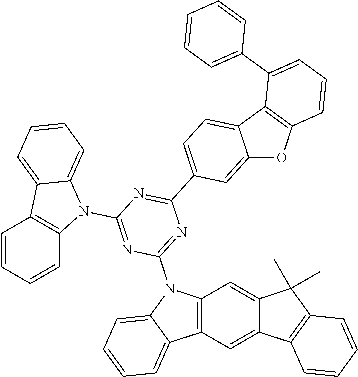 [SMILES: CC1(C)c2ccccc2-c2cc3c4ccccc4n(-c4nc(-c5ccc6c(c5)oc5cccc(-c7ccccc7)c56)nc(-n5c6ccccc6c6ccccc65)n4)c3cc21] | 493 |
| 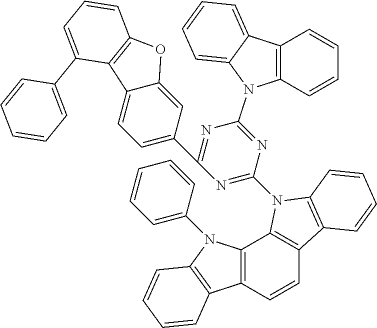 [SMILES: c1ccc(-c2cccc3oc4cc(-c5nc(-n6c7ccccc7c7ccccc76)nc(-n6c7ccccc7c7ccc8c9ccccc9n(-c9ccccc9)c8c76)n5)ccc4c23)cc1] | 494 |
| 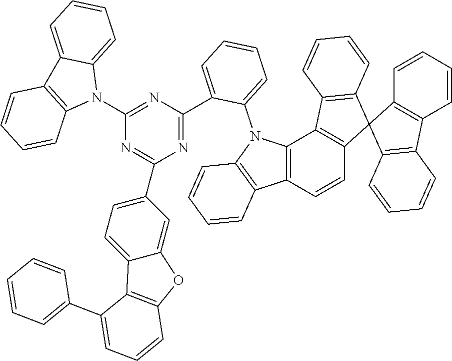 [SMILES: c1ccc(-c2cccc3oc4cc(-c5nc(-c6ccccc6-n6c7ccccc7c7ccc8c(c76)-c6ccccc6C86c7ccccc7-c7ccccc76)nc(-n6c7ccccc7c7ccccc76)n5)ccc4c23)cc1] | 495 |
| 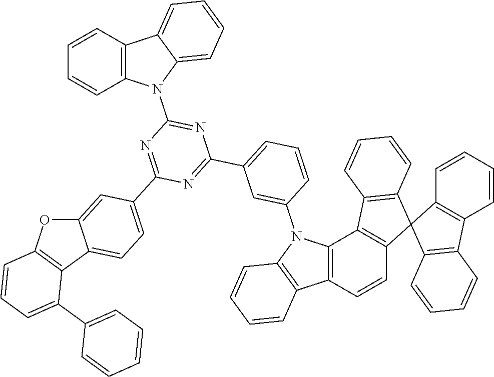 [SMILES: c1ccc(-c2cccc3oc4cc(-c5nc(-c6cccc(-n7c8ccccc8c8ccc9c(c87)-c7ccccc7C97c8ccccc8-c8ccccc87)c6)nc(-n6c7ccccc7c7ccccc76)n5)ccc4c23)cc1] | 496 |
| 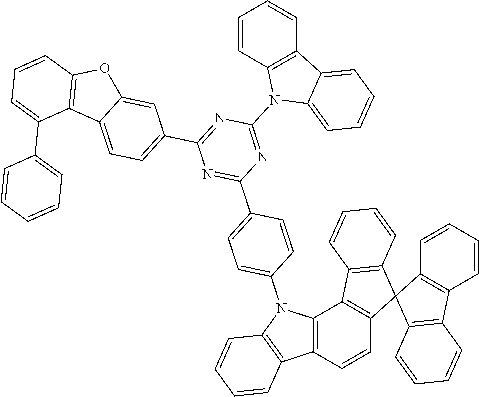 [SMILES: c1ccc(-c2cccc3oc4cc(-c5nc(-c6ccc(-n7c8ccccc8c8ccc9c(c87)-c7ccccc7C97c8ccccc8-c8ccccc87)cc6)nc(-n6c7ccccc7c7ccccc76)n5)ccc4c23)cc1] | 497 |
| 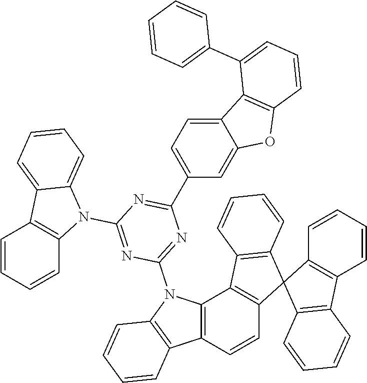 [SMILES: c1ccc(-c2cccc3oc4cc(-c5nc(-n6c7ccccc7c7ccccc76)nc(-n6c7ccccc7c7ccc8c(c76)-c6ccccc6C86c7ccccc7-c7ccccc76)n5)ccc4c23)cc1] | 498 |
| 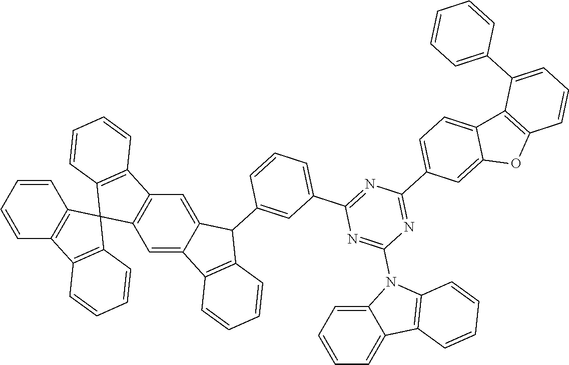 [SMILES: c1ccc(-c2cccc3oc4cc(-c5nc(-c6cccc(C7c8ccccc8-c8cc9c(cc87)-c7ccccc7C97c8ccccc8-c8ccccc87)c6)nc(-n6c7ccccc7c7ccccc76)n5)ccc4c23)cc1] | 499 |
| 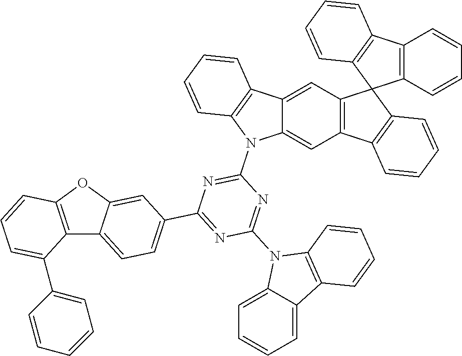 [SMILES: c1ccc(-c2cccc3oc4cc(-c5nc(-n6c7ccccc7c7ccccc76)nc(-n6c7ccccc7c7cc8c(cc76)-c6ccccc6C86c7ccccc7-c7ccccc76)n5)ccc4c23)cc1] | 500 |
| 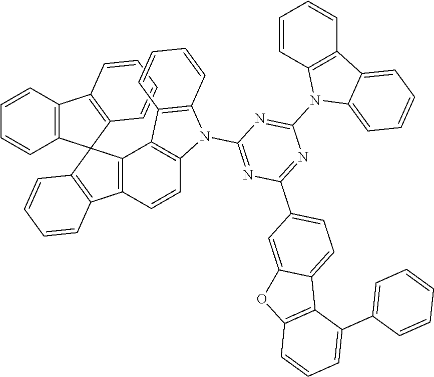 [SMILES: c1ccc(-c2cccc3oc4cc(-c5nc(-n6c7ccccc7c7ccccc76)nc(-n6c7ccccc7c7c8c(ccc76)-c6ccccc6C86c7ccccc7-c7ccccc76)n5)ccc4c23)cc1] | 501 |
| 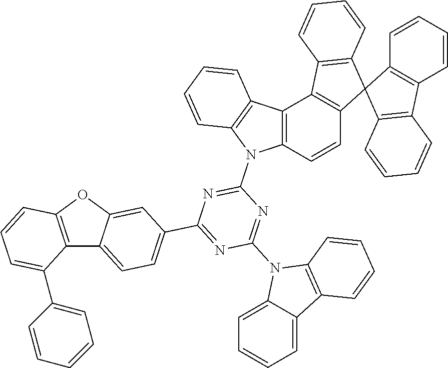 [SMILES: c1ccc(-c2cccc3oc4cc(-c5nc(-n6c7ccccc7c7ccccc76)nc(-n6c7ccccc7c7c8c(ccc76)C6(c7ccccc7-c7ccccc76)c6ccccc6-8)n5)ccc4c23)cc1] | 502 |
| 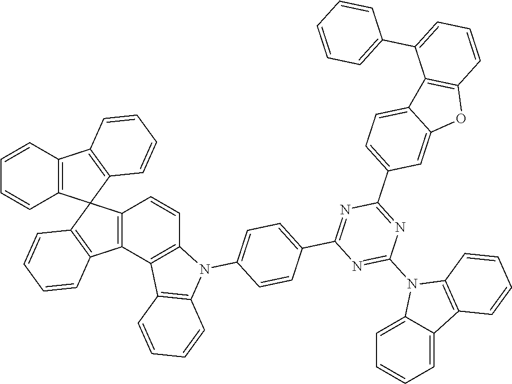 [SMILES: c1ccc(-c2cccc3oc4cc(-c5nc(-c6ccc(-n7c8ccccc8c8c9c(ccc87)C7(c8ccccc8-c8ccccc87)c7ccccc7-9)cc6)nc(-n6c7ccccc7c7ccccc76)n5)ccc4c23)cc1] | 503 |
| 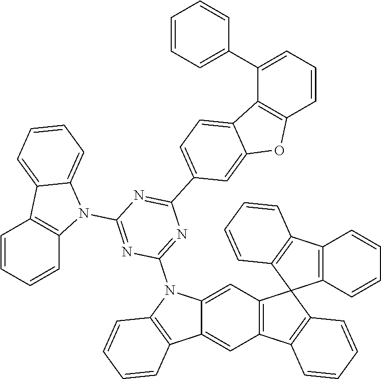 [SMILES: c1ccc(-c2cccc3oc4cc(-c5nc(-n6c7ccccc7c7ccccc76)nc(-n6c7ccccc7c7cc8c(cc76)C6(c7ccccc7-c7ccccc76)c6ccccc6-8)n5)ccc4c23)cc1] | 504 |
| 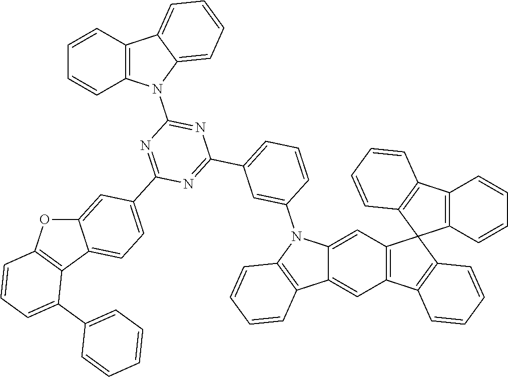 [SMILES: c1ccc(-c2cccc3oc4cc(-c5nc(-c6cccc(-n7c8ccccc8c8cc9c(cc87)C7(c8ccccc8-c8ccccc87)c7ccccc7-9)c6)nc(-n6c7ccccc7c7ccccc76)n5)ccc4c23)cc1] | 505 |
| 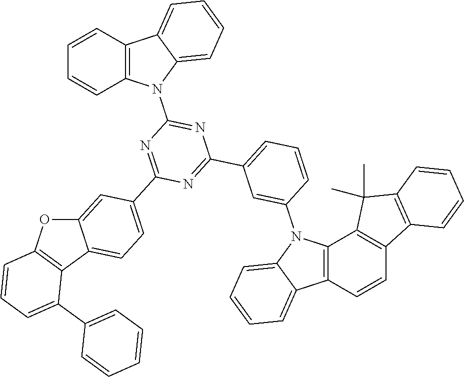 [SMILES: CC1(C)c2ccccc2-c2ccc3c4ccccc4n(-c4cccc(-c5nc(-c6ccc7c(c6)oc6cccc(-c8ccccc8)c67)nc(-n6c7ccccc7c7ccccc76)n5)c4)c3c21] | 506 |
| 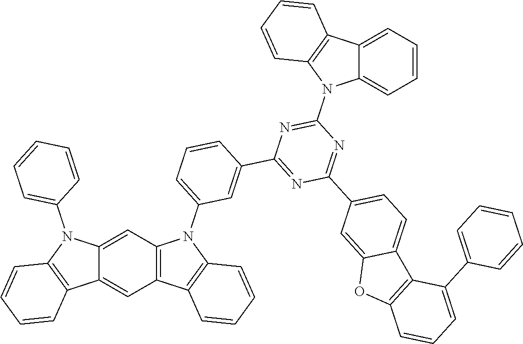 [SMILES: c1ccc(-c2cccc3oc4cc(-c5nc(-c6cccc(-n7c8ccccc8c8cc9c%10ccccc%10n(-c%10ccccc%10)c9cc87)c6)nc(-n6c7ccccc7c7ccccc76)n5)ccc4c23)cc1] | 507 |
| 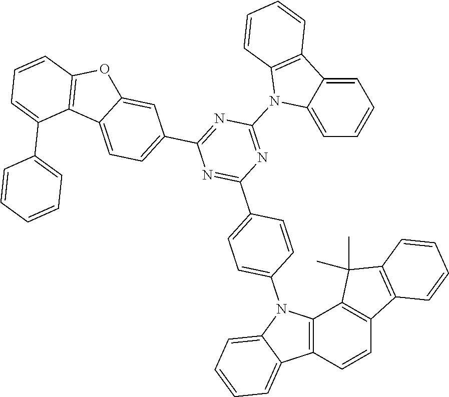 [SMILES: CC1(C)c2ccccc2-c2ccc3c4ccccc4n(-c4ccc(-c5nc(-c6ccc7c(c6)oc6cccc(-c8ccccc8)c67)nc(-n6c7ccccc7c7ccccc76)n5)cc4)c3c21] | 508 |
| 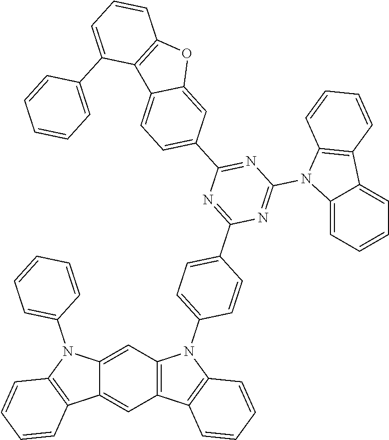 [SMILES: c1ccc(-c2cccc3oc4cc(-c5nc(-c6ccc(-n7c8ccccc8c8cc9c%10ccccc%10n(-c%10ccccc%10)c9cc87)cc6)nc(-n6c7ccccc7c7ccccc76)n5)ccc4c23)cc1] | 509 |
| 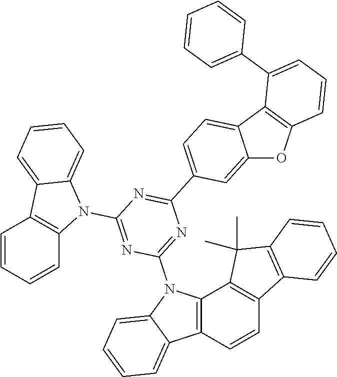 [SMILES: CC1(C)c2ccccc2-c2ccc3c4ccccc4n(-c4nc(-c5ccc6c(c5)oc5cccc(-c7ccccc7)c56)nc(-n5c6ccccc6c6ccccc65)n4)c3c21] | 510 |
| 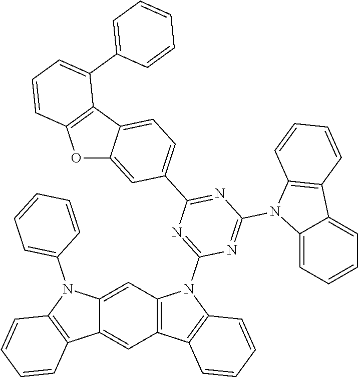 [SMILES: c1ccc(-c2cccc3oc4cc(-c5nc(-n6c7ccccc7c7ccccc76)nc(-n6c7ccccc7c7cc8c9ccccc9n(-c9ccccc9)c8cc76)n5)ccc4c23)cc1] | 511 |
| 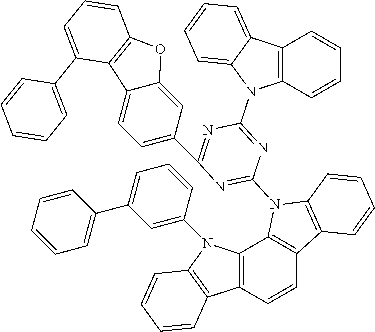 [SMILES: c1ccc(-c2cccc(-n3c4ccccc4c4ccc5c6ccccc6n(-c6nc(-c7ccc8c(c7)oc7cccc(-c9ccccc9)c78)nc(-n7c8ccccc8c8ccccc87)n6)c5c43)c2)cc1] | 512 |
| 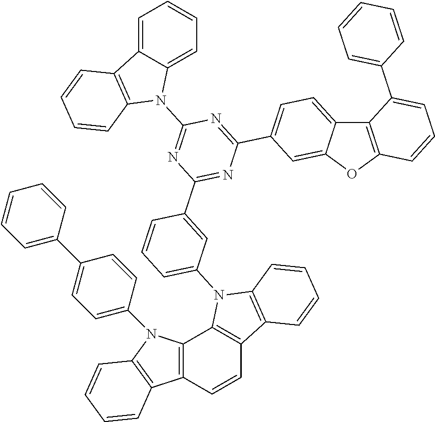 [SMILES: c1ccc(-c2ccc(-n3c4ccccc4c4ccc5c6ccccc6n(-c6cccc(-c7nc(-c8ccc9c(c8)oc8cccc(-c%10ccccc%10)c89)nc(-n8c9ccccc9c9ccccc98)n7)c6)c5c43)cc2)cc1] | 513 |
| 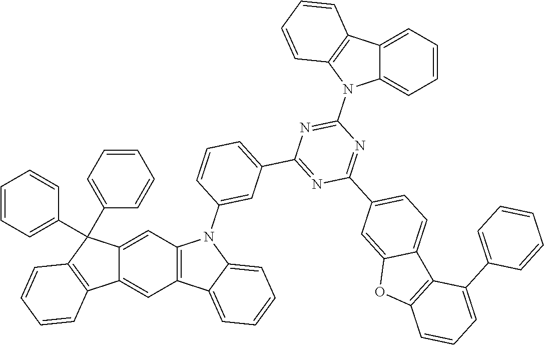 [SMILES: c1ccc(-c2cccc3oc4cc(-c5nc(-c6cccc(-n7c8ccccc8c8cc9c(cc87)C(c7ccccc7)(c7ccccc7)c7ccccc7-9)c6)nc(-n6c7ccccc7c7ccccc76)n5)ccc4c23)cc1] | 514 |
| 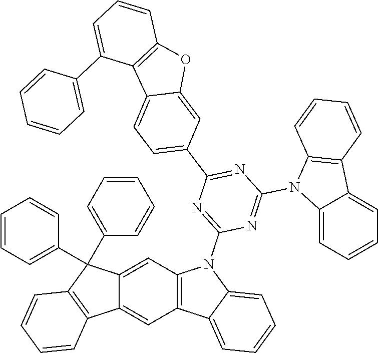 [SMILES: c1ccc(-c2cccc3oc4cc(-c5nc(-n6c7ccccc7c7ccccc76)nc(-n6c7ccccc7c7cc8c(cc76)C(c6ccccc6)(c6ccccc6)c6ccccc6-8)n5)ccc4c23)cc1] | 515 |
| 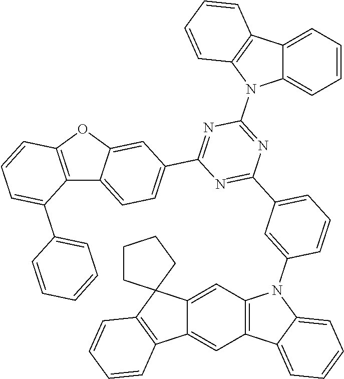 [SMILES: c1ccc(-c2cccc3oc4cc(-c5nc(-c6cccc(-n7c8ccccc8c8cc9c(cc87)C7(CCCC7)c7ccccc7-9)c6)nc(-n6c7ccccc7c7ccccc76)n5)ccc4c23)cc1] | 516 |
| 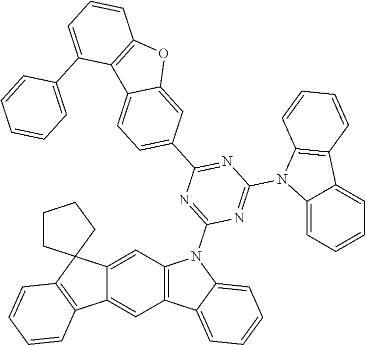 [SMILES: c1ccc(-c2cccc3oc4cc(-c5nc(-n6c7ccccc7c7ccccc76)nc(-n6c7ccccc7c7cc8c(cc76)C6(CCCC6)c6ccccc6-8)n5)ccc4c23)cc1] | 517 |
| 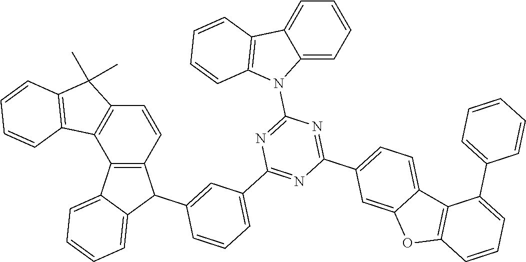 [SMILES: CC1(C)c2ccccc2-c2c1ccc1c2-c2ccccc2C1c1cccc(-c2nc(-c3ccc4c(c3)oc3cccc(-c5ccccc5)c34)nc(-n3c4ccccc4c4ccccc43)n2)c1] | 518 |
| 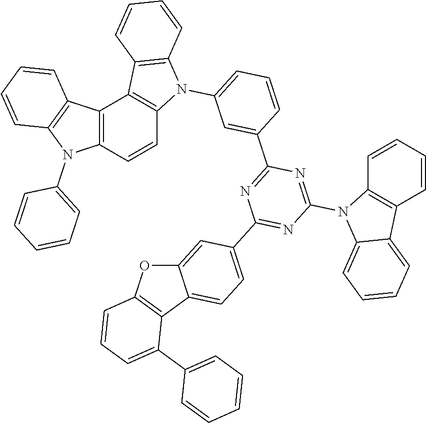 [SMILES: c1ccc(-c2cccc3oc4cc(-c5nc(-c6cccc(-n7c8ccccc8c8c9c%10ccccc%10n(-c%10ccccc%10)c9ccc87)c6)nc(-n6c7ccccc7c7ccccc76)n5)ccc4c23)cc1] | 519 |
| 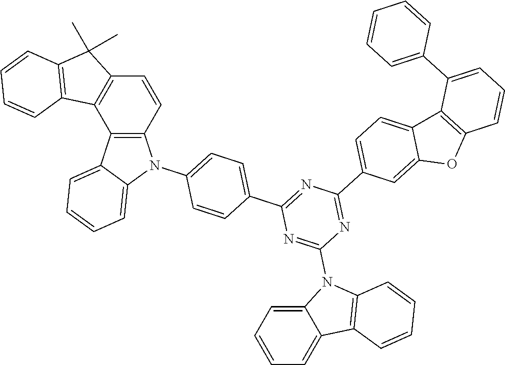 [SMILES: CC1(C)c2ccccc2-c2c1ccc1c2c2ccccc2n1-c1ccc(-c2nc(-c3ccc4c(c3)oc3cccc(-c5ccccc5)c34)nc(-n3c4ccccc4c4ccccc43)n2)cc1] | 520 |
| 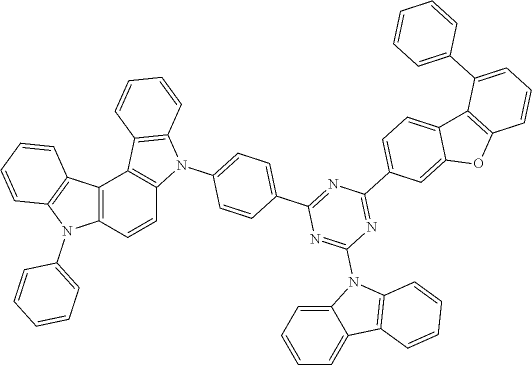 [SMILES: c1ccc(-c2cccc3oc4cc(-c5nc(-c6ccc(-n7c8ccccc8c8c9c%10ccccc%10n(-c%10ccccc%10)c9ccc87)cc6)nc(-n6c7ccccc7c7ccccc76)n5)ccc4c23)cc1] | 521 |
| 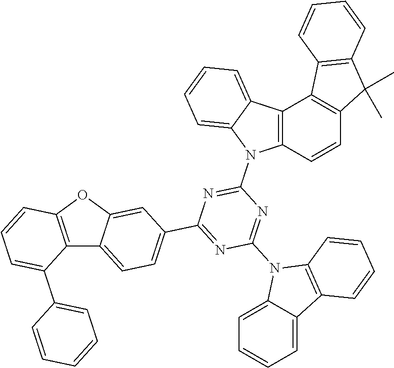 [SMILES: CC1(C)c2ccccc2-c2c1ccc1c2c2ccccc2n1-c1nc(-c2ccc3c(c2)oc2cccc(-c4ccccc4)c23)nc(-n2c3ccccc3c3ccccc32)n1] | 522 |
| 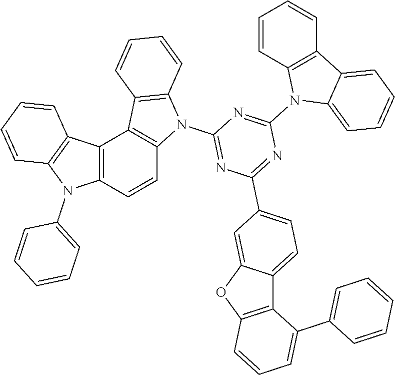 [SMILES: c1ccc(-c2cccc3oc4cc(-c5nc(-n6c7ccccc7c7ccccc76)nc(-n6c7ccccc7c7c8c9ccccc9n(-c9ccccc9)c8ccc76)n5)ccc4c23)cc1] | 523 |
| 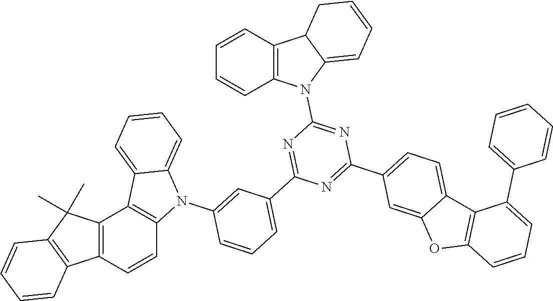 [SMILES: CC1(C)c2ccccc2-c2ccc3c(c21)c1ccccc1n3-c1cccc(-c2nc(-c3ccc4c(c3)oc3cccc(-c5ccccc5)c34)nc(N3C4=CC=CCC4c4ccccc43)n2)c1] | 524 |
| 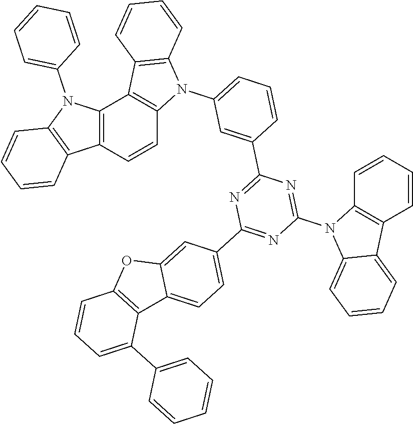 [SMILES: c1ccc(-c2cccc3oc4cc(-c5nc(-c6cccc(-n7c8ccccc8c8c7ccc7c9ccccc9n(-c9ccccc9)c78)c6)nc(-n6c7ccccc7c7ccccc76)n5)ccc4c23)cc1] | 525 |
| 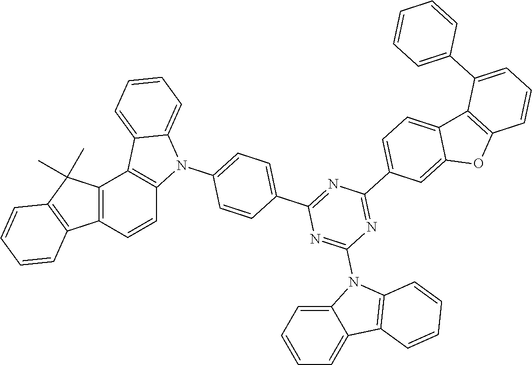 [SMILES: CC1(C)c2ccccc2-c2ccc3c(c21)c1ccccc1n3-c1ccc(-c2nc(-c3ccc4c(c3)oc3cccc(-c5ccccc5)c34)nc(-n3c4ccccc4c4ccccc43)n2)cc1] | 526 |
| 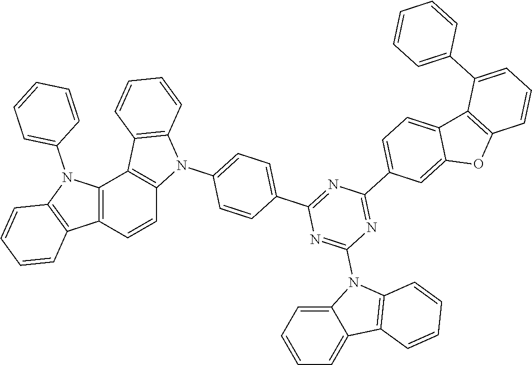 [SMILES: c1ccc(-c2cccc3oc4cc(-c5nc(-c6ccc(-n7c8ccccc8c8c7ccc7c9ccccc9n(-c9ccccc9)c78)cc6)nc(-n6c7ccccc7c7ccccc76)n5)ccc4c23)cc1] | 527 |
| 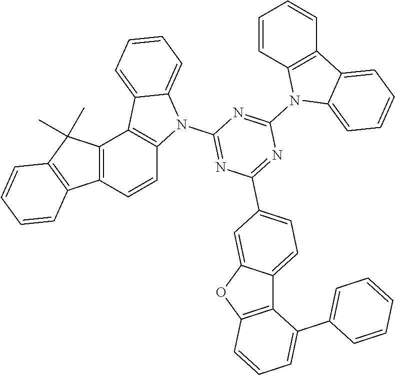 [SMILES: CC1(C)c2ccccc2-c2ccc3c(c21)c1ccccc1n3-c1nc(-c2ccc3c(c2)oc2cccc(-c4ccccc4)c23)nc(-n2c3ccccc3c3ccccc32)n1] | 528 |
| 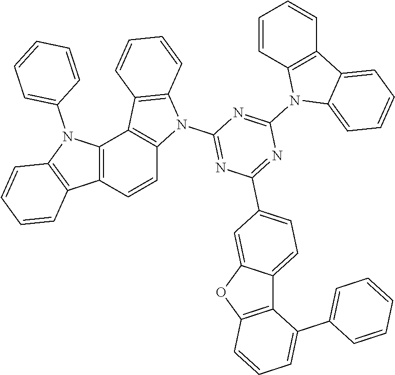 [SMILES: c1ccc(-c2cccc3oc4cc(-c5nc(-n6c7ccccc7c7ccccc76)nc(-n6c7ccccc7c7c6ccc6c8ccccc8n(-c8ccccc8)c67)n5)ccc4c23)cc1] | 529 |
| 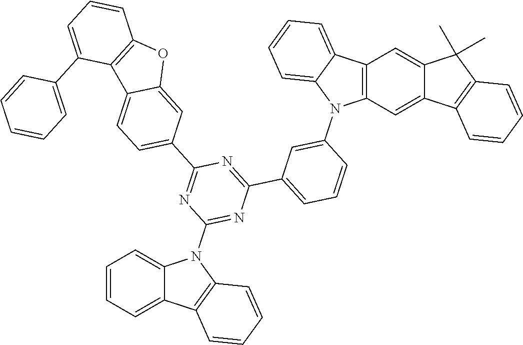 [SMILES: CC1(C)c2ccccc2-c2cc3c(cc21)c1ccccc1n3-c1cccc(-c2nc(-c3ccc4c(c3)oc3cccc(-c5ccccc5)c34)nc(-n3c4ccccc4c4ccccc43)n2)c1] | 530 |
| 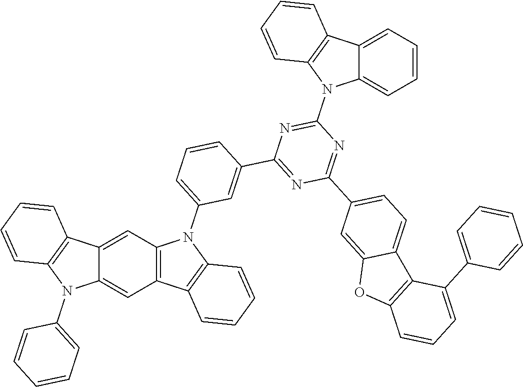 [SMILES: c1ccc(-c2cccc3oc4cc(-c5nc(-c6cccc(-n7c8ccccc8c8cc9c(cc87)c7ccccc7n9-c7ccccc7)c6)nc(-n6c7ccccc7c7ccccc76)n5)ccc4c23)cc1] | 531 |
| 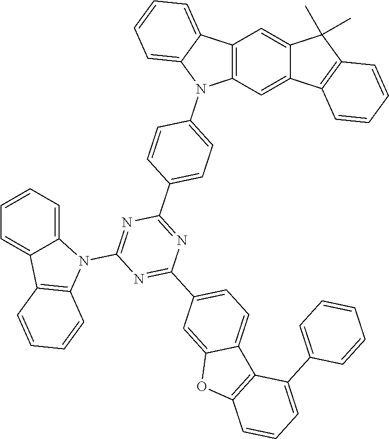 [SMILES: CC1(C)c2ccccc2-c2cc3c(cc21)c1ccccc1n3-c1ccc(-c2nc(-c3ccc4c(c3)oc3cccc(-c5ccccc5)c34)nc(-n3c4ccccc4c4ccccc43)n2)cc1] | 532 |
| 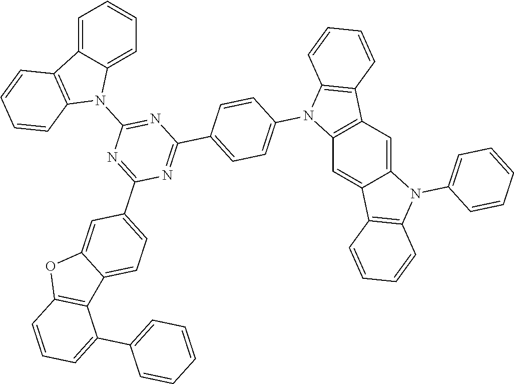 [SMILES: c1ccc(-c2cccc3oc4cc(-c5nc(-c6ccc(-n7c8ccccc8c8cc9c(cc87)c7ccccc7n9-c7ccccc7)cc6)nc(-n6c7ccccc7c7ccccc76)n5)ccc4c23)cc1] | 533 |
| 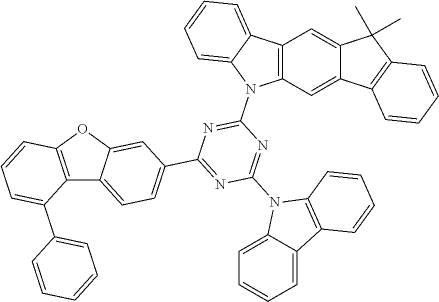 [SMILES: CC1(C)c2ccccc2-c2cc3c(cc21)c1ccccc1n3-c1nc(-c2ccc3c(c2)oc2cccc(-c4ccccc4)c23)nc(-n2c3ccccc3c3ccccc32)n1] | 534 |
| 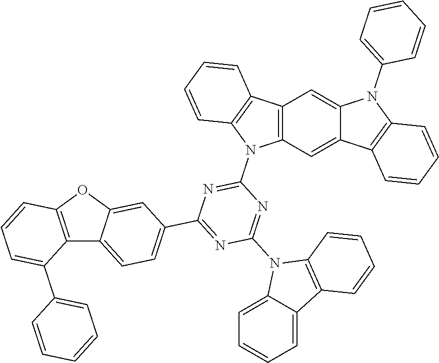 [SMILES: c1ccc(-c2cccc3oc4cc(-c5nc(-n6c7ccccc7c7ccccc76)nc(-n6c7ccccc7c7cc8c(cc76)c6ccccc6n8-c6ccccc6)n5)ccc4c23)cc1] | 535 |
| 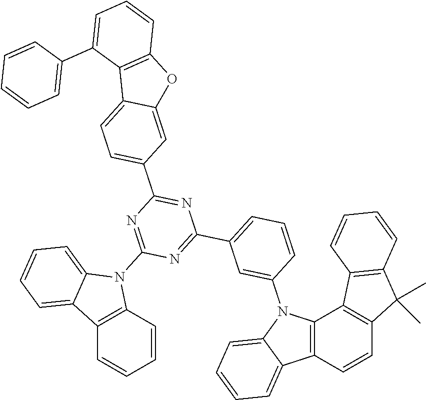 [SMILES: CC1(C)c2ccccc2-c2c1ccc1c3ccccc3n(-c3cccc(-c4nc(-c5ccc6c(c5)oc5cccc(-c7ccccc7)c56)nc(-n5c6ccccc6c6ccccc65)n4)c3)c21] | 536 |
| 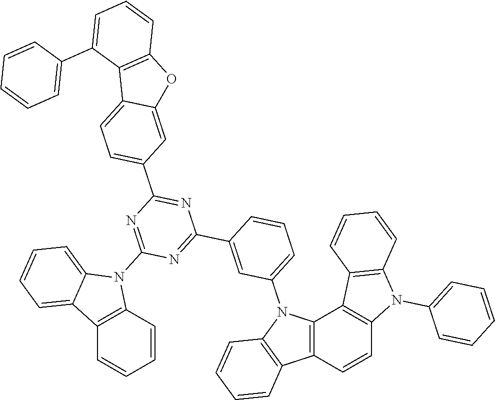 [SMILES: c1ccc(-c2cccc3oc4cc(-c5nc(-c6cccc(-n7c8ccccc8c8ccc9c(c%10ccccc%10n9-c9ccccc9)c87)c6)nc(-n6c7ccccc7c7ccccc76)n5)ccc4c23)cc1] | 537 |
| 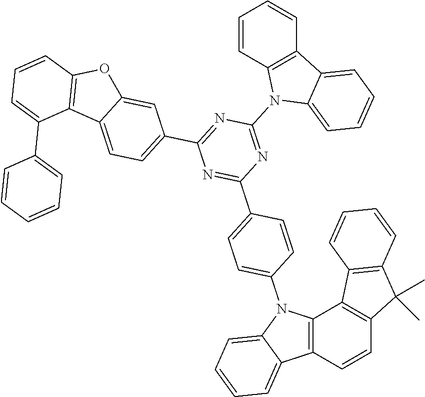 [SMILES: CC1(C)c2ccccc2-c2c1ccc1c3ccccc3n(-c3ccc(-c4nc(-c5ccc6c(c5)oc5cccc(-c7ccccc7)c56)nc(-n5c6ccccc6c6ccccc65)n4)cc3)c21] | 538 |
| 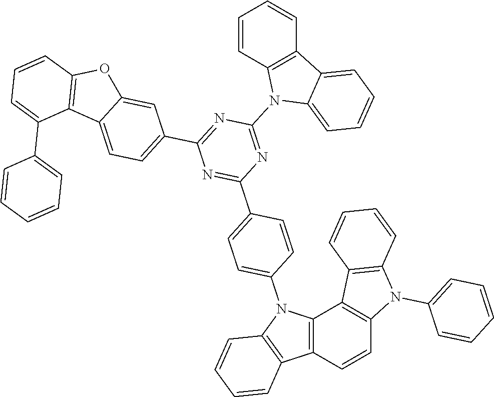 [SMILES: c1ccc(-c2cccc3oc4cc(-c5nc(-c6ccc(-n7c8ccccc8c8ccc9c(c%10ccccc%10n9-c9ccccc9)c87)cc6)nc(-n6c7ccccc7c7ccccc76)n5)ccc4c23)cc1] | 539 |
| 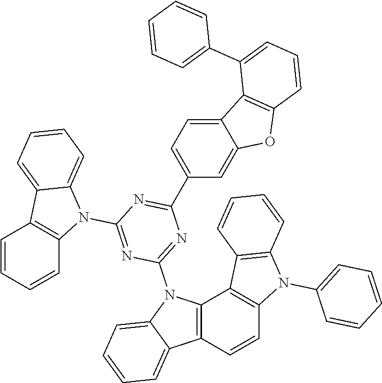 [SMILES: c1ccc(-c2cccc3oc4cc(-c5nc(-n6c7ccccc7c7ccccc76)nc(-n6c7ccccc7c7ccc8c(c9ccccc9n8-c8ccccc8)c76)n5)ccc4c23)cc1] | 540 |
| 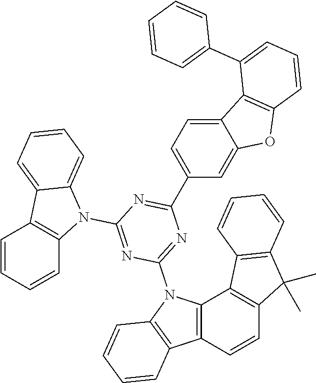 [SMILES: CC1(C)c2ccccc2-c2c1ccc1c3ccccc3n(-c3nc(-c4ccc5c(c4)oc4cccc(-c6ccccc6)c45)nc(-n4c5ccccc5c5ccccc54)n3)c21] | 541 |
| 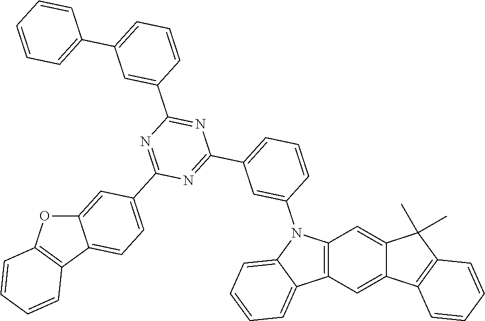 [SMILES: CC1(C)c2ccccc2-c2cc3c4ccccc4n(-c4cccc(-c5nc(-c6cccc(-c7ccccc7)c6)nc(-c6ccc7c(c6)oc6ccccc67)n5)c4)c3cc21] | 542 |
| 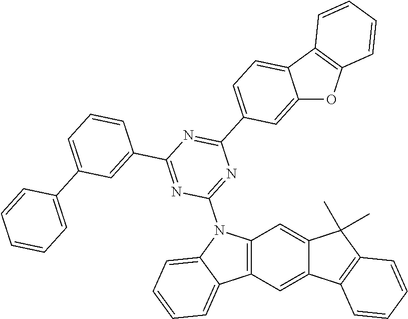 [SMILES: CC1(C)c2ccccc2-c2cc3c4ccccc4n(-c4nc(-c5cccc(-c6ccccc6)c5)nc(-c5ccc6c(c5)oc5ccccc56)n4)c3cc21] | 543 |
| 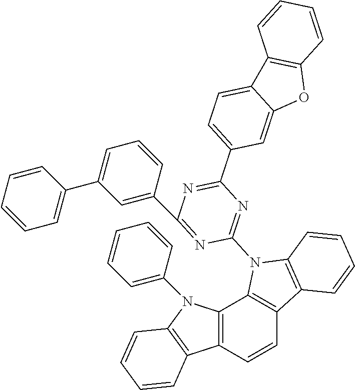 [SMILES: c1ccc(-c2cccc(-c3nc(-c4ccc5c(c4)oc4ccccc45)nc(-n4c5ccccc5c5ccc6c7ccccc7n(-c7ccccc7)c6c54)n3)c2)cc1] | 544 |
| 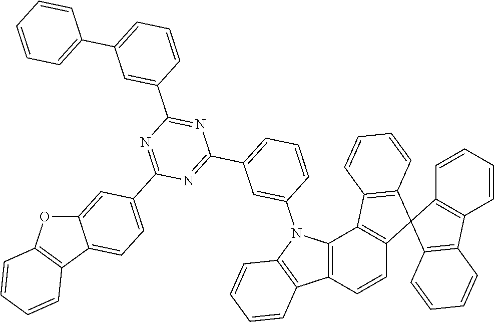 [SMILES: c1ccc(-c2cccc(-c3nc(-c4cccc(-n5c6ccccc6c6ccc7c(c65)-c5ccccc5C75c6ccccc6-c6ccccc65)c4)nc(-c4ccc5c(c4)oc4ccccc45)n3)c2)cc1] | 545 |
| 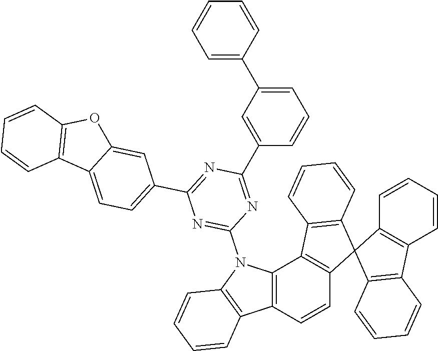 [SMILES: c1ccc(-c2cccc(-c3nc(-c4ccc5c(c4)oc4ccccc45)nc(-n4c5ccccc5c5ccc6c(c54)-c4ccccc4C64c5ccccc5-c5ccccc54)n3)c2)cc1] | 546 |
| 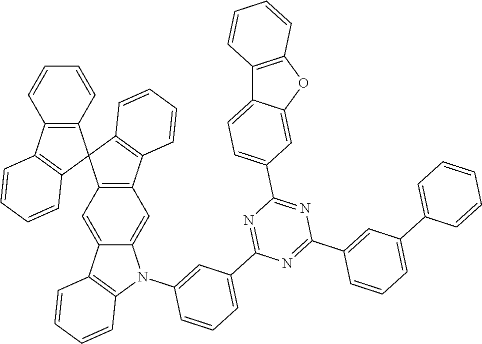 [SMILES: c1ccc(-c2cccc(-c3nc(-c4cccc(-n5c6ccccc6c6cc7c(cc65)-c5ccccc5C75c6ccccc6-c6ccccc65)c4)nc(-c4ccc5c(c4)oc4ccccc45)n3)c2)cc1] | 547 |
| 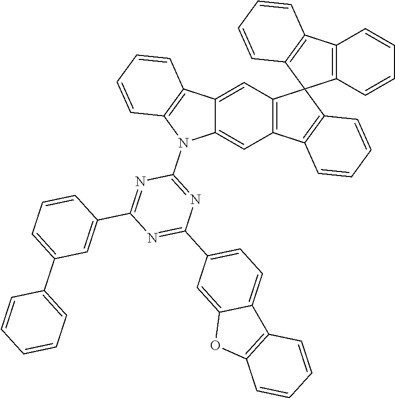 [SMILES: c1ccc(-c2cccc(-c3nc(-c4ccc5c(c4)oc4ccccc45)nc(-n4c5ccccc5c5cc6c(cc54)-c4ccccc4C64c5ccccc5-c5ccccc54)n3)c2)cc1] | 548 |
| 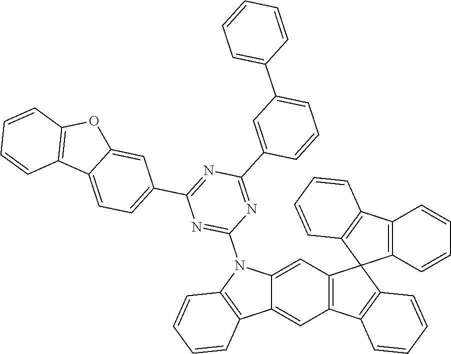 [SMILES: c1ccc(-c2cccc(-c3nc(-c4ccc5c(c4)oc4ccccc45)nc(-n4c5ccccc5c5cc6c(cc54)C4(c5ccccc5-c5ccccc54)c4ccccc4-6)n3)c2)cc1] | 549 |
| 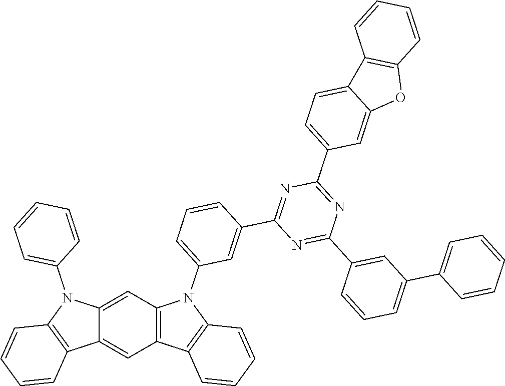 [SMILES: c1ccc(-c2cccc(-c3nc(-c4cccc(-n5c6ccccc6c6cc7c8ccccc8n(-c8ccccc8)c7cc65)c4)nc(-c4ccc5c(c4)oc4ccccc45)n3)c2)cc1] | 550 |
| 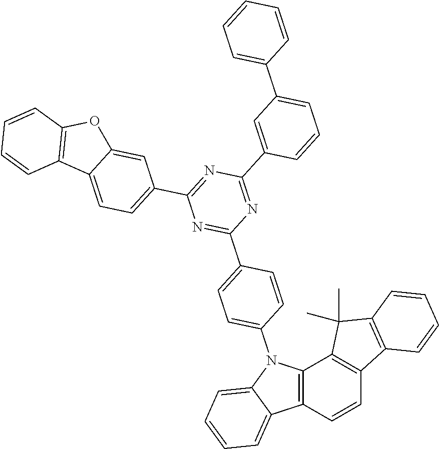 [SMILES: CC1(C)c2ccccc2-c2ccc3c4ccccc4n(-c4ccc(-c5nc(-c6cccc(-c7ccccc7)c6)nc(-c6ccc7c(c6)oc6ccccc67)n5)cc4)c3c21] | 551 |
| 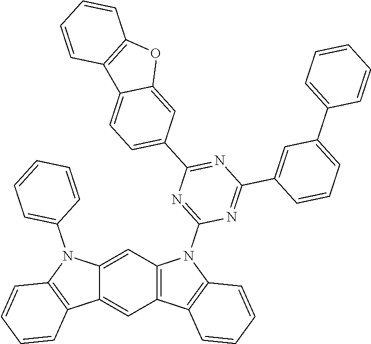 [SMILES: c1ccc(-c2cccc(-c3nc(-c4ccc5c(c4)oc4ccccc45)nc(-n4c5ccccc5c5cc6c7ccccc7n(-c7ccccc7)c6cc54)n3)c2)cc1] | 552 |
| 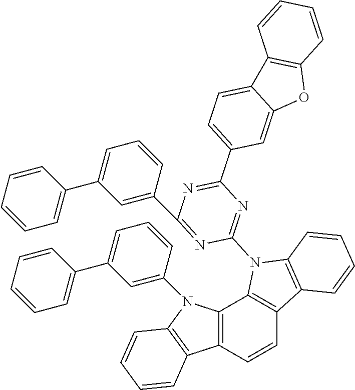 [SMILES: c1ccc(-c2cccc(-c3nc(-c4ccc5c(c4)oc4ccccc45)nc(-n4c5ccccc5c5ccc6c7ccccc7n(-c7cccc(-c8ccccc8)c7)c6c54)n3)c2)cc1] | 553 |
| 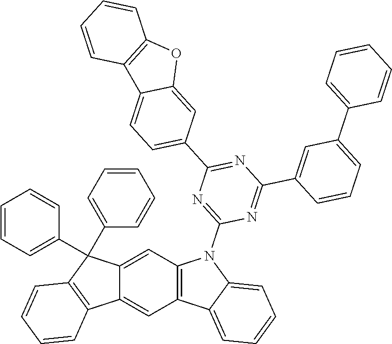 [SMILES: c1ccc(-c2cccc(-c3nc(-c4ccc5c(c4)oc4ccccc45)nc(-n4c5ccccc5c5cc6c(cc54)C(c4ccccc4)(c4ccccc4)c4ccccc4-6)n3)c2)cc1] | 554 |
| 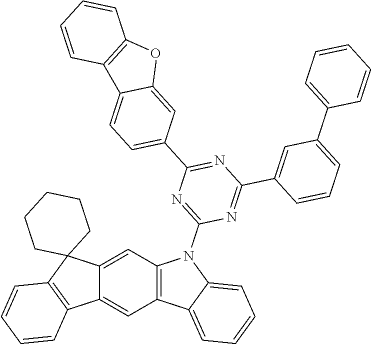 [SMILES: c1ccc(-c2cccc(-c3nc(-c4ccc5c(c4)oc4ccccc45)nc(-n4c5ccccc5c5cc6c(cc54)C4(CCCCC4)c4ccccc4-6)n3)c2)cc1] | 555 |
| 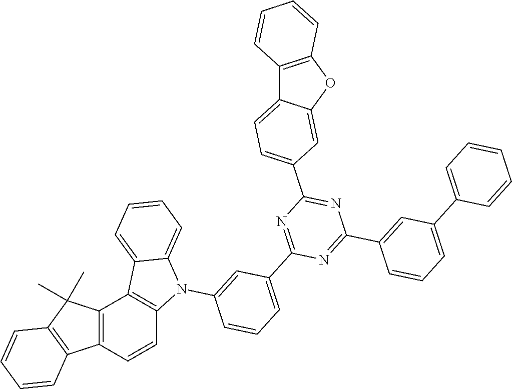 [SMILES: CC1(C)c2ccccc2-c2ccc3c(c21)c1ccccc1n3-c1cccc(-c2nc(-c3cccc(-c4ccccc4)c3)nc(-c3ccc4c(c3)oc3ccccc34)n2)c1] | 556 |
| 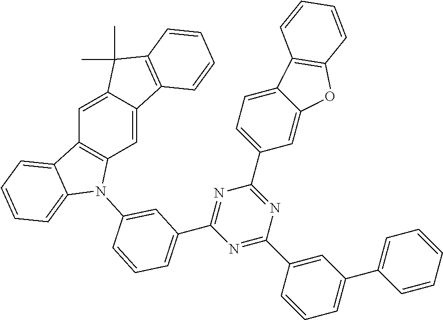 [SMILES: CC1(C)c2ccccc2-c2cc3c(cc21)c1ccccc1n3-c1cccc(-c2nc(-c3cccc(-c4ccccc4)c3)nc(-c3ccc4c(c3)oc3ccccc34)n2)c1] | 557 |
| 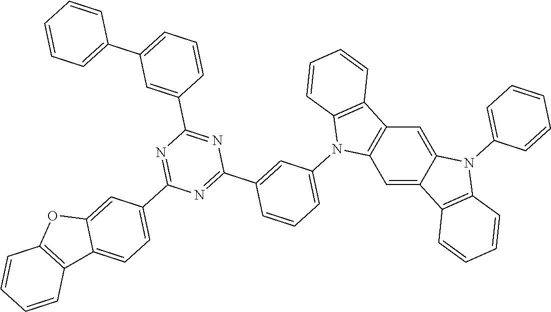 [SMILES: c1ccc(-c2cccc(-c3nc(-c4cccc(-n5c6ccccc6c6cc7c(cc65)c5ccccc5n7-c5ccccc5)c4)nc(-c4ccc5c(c4)oc4ccccc45)n3)c2)cc1] | 558 |
| 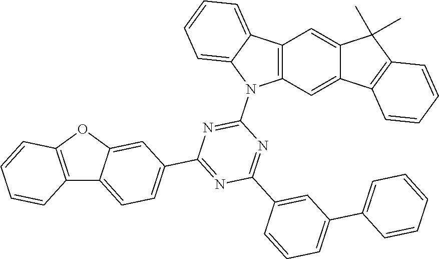 [SMILES: CC1(C)c2ccccc2-c2cc3c(cc21)c1ccccc1n3-c1nc(-c2cccc(-c3ccccc3)c2)nc(-c2ccc3c(c2)oc2ccccc23)n1] | 559 |
| 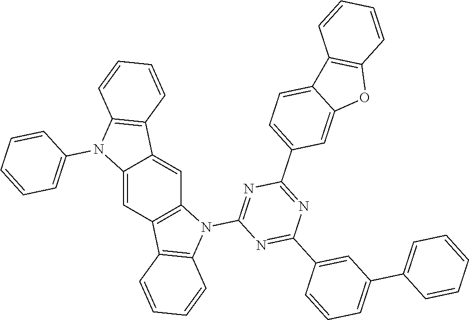 [SMILES: c1ccc(-c2cccc(-c3nc(-c4ccc5c(c4)oc4ccccc45)nc(-n4c5ccccc5c5cc6c(cc54)c4ccccc4n6-c4ccccc4)n3)c2)cc1] | 560 |
| 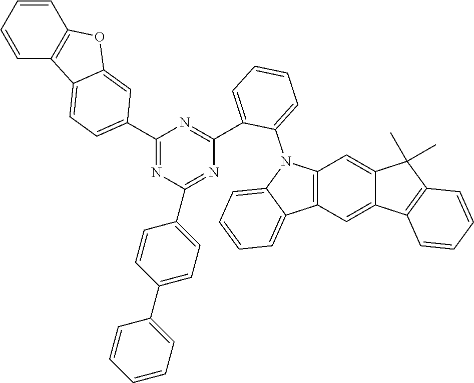 [SMILES: CC1(C)c2ccccc2-c2cc3c4ccccc4n(-c4ccccc4-c4nc(-c5ccc(-c6ccccc6)cc5)nc(-c5ccc6c(c5)oc5ccccc56)n4)c3cc21] | 561 |
| 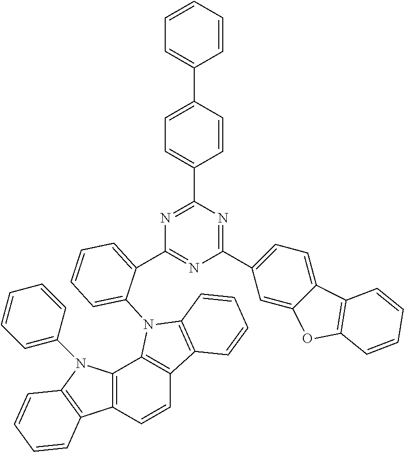 [SMILES: c1ccc(-c2ccc(-c3nc(-c4ccc5c(c4)oc4ccccc45)nc(-c4ccccc4-n4c5ccccc5c5ccc6c7ccccc7n(-c7ccccc7)c6c54)n3)cc2)cc1] | 562 |
| 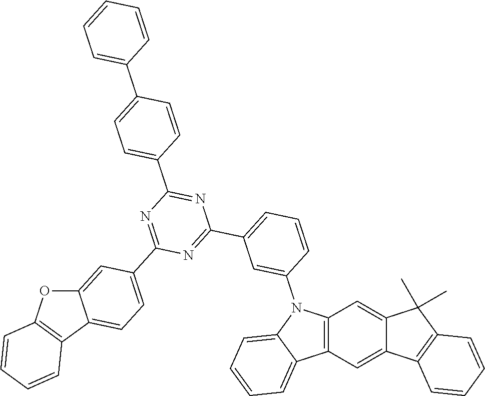 [SMILES: CC1(C)c2ccccc2-c2cc3c4ccccc4n(-c4cccc(-c5nc(-c6ccc(-c7ccccc7)cc6)nc(-c6ccc7c(c6)oc6ccccc67)n5)c4)c3cc21] | 563 |
| 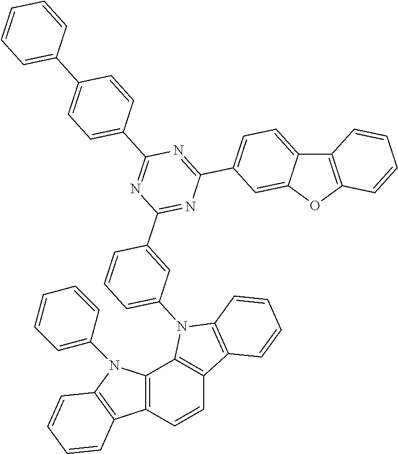 [SMILES: c1ccc(-c2ccc(-c3nc(-c4cccc(-n5c6ccccc6c6ccc7c8ccccc8n(-c8ccccc8)c7c65)c4)nc(-c4ccc5c(c4)oc4ccccc45)n3)cc2)cc1] | 564 |
| 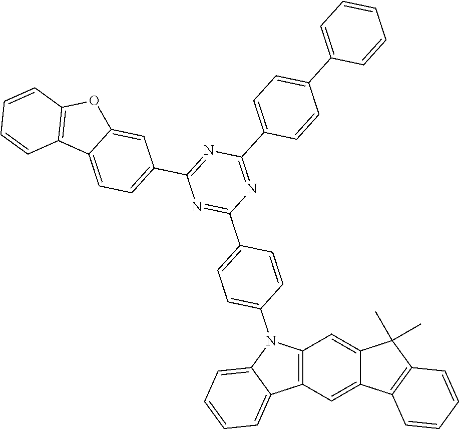 [SMILES: CC1(C)c2ccccc2-c2cc3c4ccccc4n(-c4ccc(-c5nc(-c6ccc(-c7ccccc7)cc6)nc(-c6ccc7c(c6)oc6ccccc67)n5)cc4)c3cc21] | 565 |
| 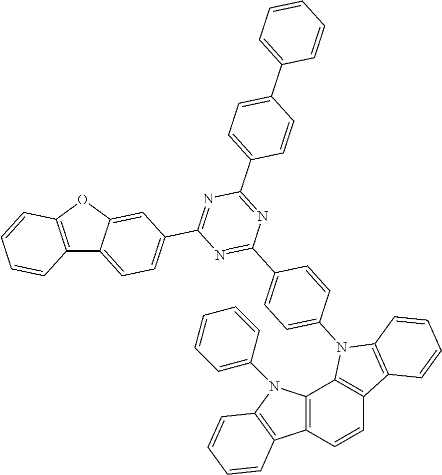 [SMILES: c1ccc(-c2ccc(-c3nc(-c4ccc(-n5c6ccccc6c6ccc7c8ccccc8n(-c8ccccc8)c7c65)cc4)nc(-c4ccc5c(c4)oc4ccccc45)n3)cc2)cc1] | 566 |
| 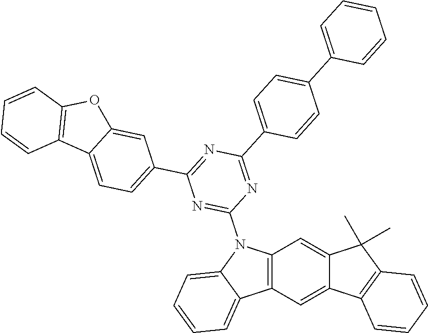 [SMILES: CC1(C)c2ccccc2-c2cc3c4ccccc4n(-c4nc(-c5ccc(-c6ccccc6)cc5)nc(-c5ccc6c(c5)oc5ccccc56)n4)c3cc21] | 567 |
| 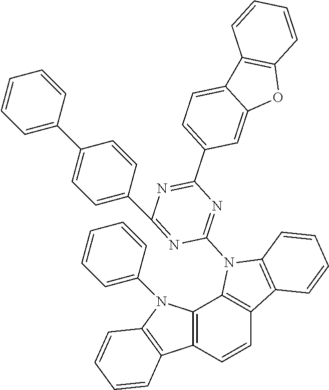 [SMILES: c1ccc(-c2ccc(-c3nc(-c4ccc5c(c4)oc4ccccc45)nc(-n4c5ccccc5c5ccc6c7ccccc7n(-c7ccccc7)c6c54)n3)cc2)cc1] | 568 |
| 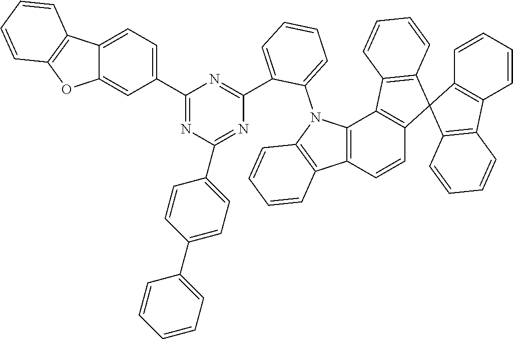 [SMILES: c1ccc(-c2ccc(-c3nc(-c4ccc5c(c4)oc4ccccc45)nc(-c4ccccc4-n4c5ccccc5c5ccc6c(c54)-c4ccccc4C64c5ccccc5-c5ccccc54)n3)cc2)cc1] | 569 |
| 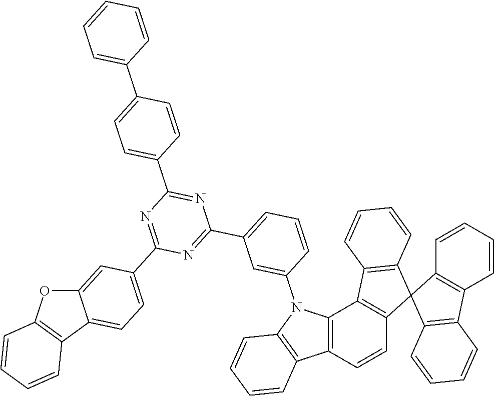 [SMILES: c1ccc(-c2ccc(-c3nc(-c4cccc(-n5c6ccccc6c6ccc7c(c65)-c5ccccc5C75c6ccccc6-c6ccccc65)c4)nc(-c4ccc5c(c4)oc4ccccc45)n3)cc2)cc1] | 570 |
| 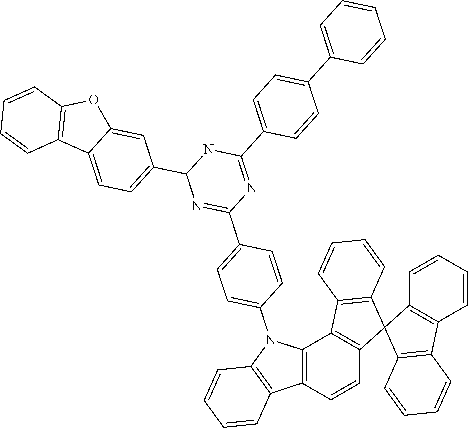 [SMILES: c1ccc(-c2ccc(C3=NC(c4ccc(-n5c6ccccc6c6ccc7c(c65)-c5ccccc5C75c6ccccc6-c6ccccc65)cc4)=NC(c4ccc5c(c4)oc4ccccc45)N3)cc2)cc1] | 571 |
| 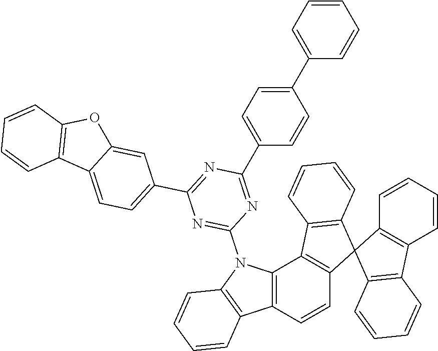 [SMILES: c1ccc(-c2ccc(-c3nc(-c4ccc5c(c4)oc4ccccc45)nc(-n4c5ccccc5c5ccc6c(c54)-c4ccccc4C64c5ccccc5-c5ccccc54)n3)cc2)cc1] | 572 |
| 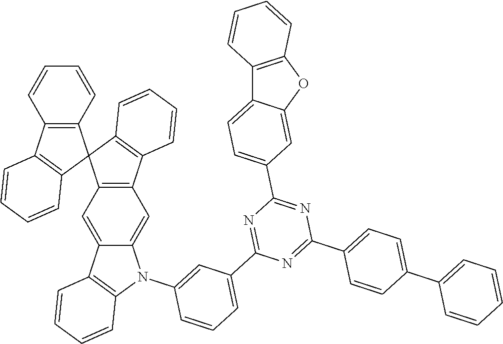 [SMILES: c1ccc(-c2ccc(-c3nc(-c4cccc(-n5c6ccccc6c6cc7c(cc65)-c5ccccc5C75c6ccccc6-c6ccccc65)c4)nc(-c4ccc5c(c4)oc4ccccc45)n3)cc2)cc1] | 573 |
| 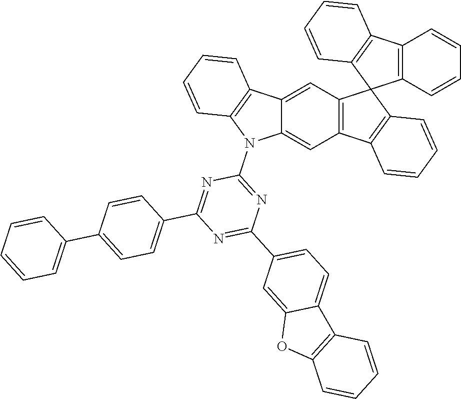 [SMILES: c1ccc(-c2ccc(-c3nc(-c4ccc5c(c4)oc4ccccc45)nc(-n4c5ccccc5c5cc6c(cc54)-c4ccccc4C64c5ccccc5-c5ccccc54)n3)cc2)cc1] | 574 |
| 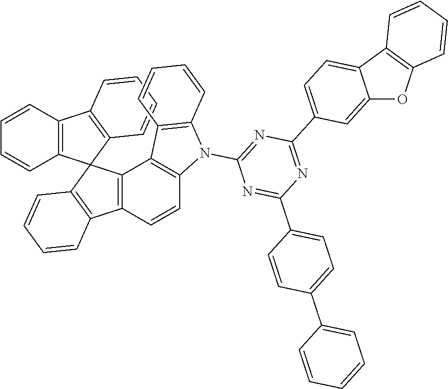 [SMILES: c1ccc(-c2ccc(-c3nc(-c4ccc5c(c4)oc4ccccc45)nc(-n4c5ccccc5c5c6c(ccc54)-c4ccccc4C64c5ccccc5-c5ccccc54)n3)cc2)cc1] | 575 |
| 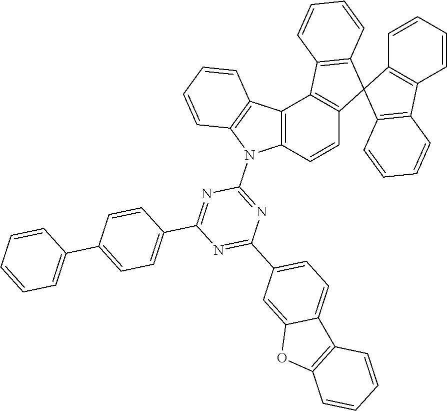 [SMILES: c1ccc(-c2ccc(-c3nc(-c4ccc5c(c4)oc4ccccc45)nc(-n4c5ccccc5c5c6c(ccc54)C4(c5ccccc5-c5ccccc54)c4ccccc4-6)n3)cc2)cc1] | 576 |
| 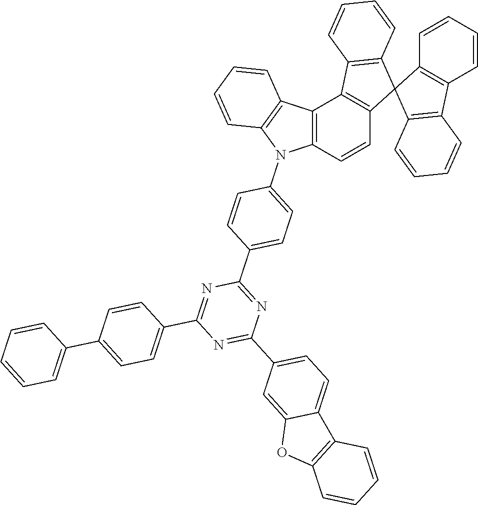 [SMILES: c1ccc(-c2ccc(-c3nc(-c4ccc(-n5c6ccccc6c6c7c(ccc65)C5(c6ccccc6-c6ccccc65)c5ccccc5-7)cc4)nc(-c4ccc5c(c4)oc4ccccc45)n3)cc2)cc1] | 577 |
| 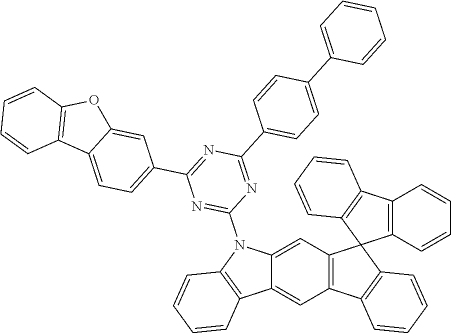 [SMILES: c1ccc(-c2ccc(-c3nc(-c4ccc5c(c4)oc4ccccc45)nc(-n4c5ccccc5c5cc6c(cc54)C4(c5ccccc5-c5ccccc54)c4ccccc4-6)n3)cc2)cc1] | 578 |
| 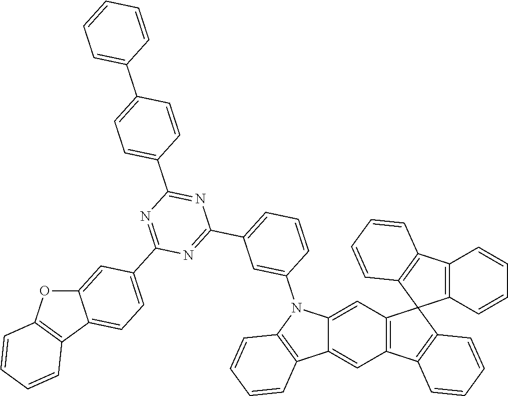 [SMILES: c1ccc(-c2ccc(-c3nc(-c4cccc(-n5c6ccccc6c6cc7c(cc65)C5(c6ccccc6-c6ccccc65)c5ccccc5-7)c4)nc(-c4ccc5c(c4)oc4ccccc45)n3)cc2)cc1] | 579 |
| 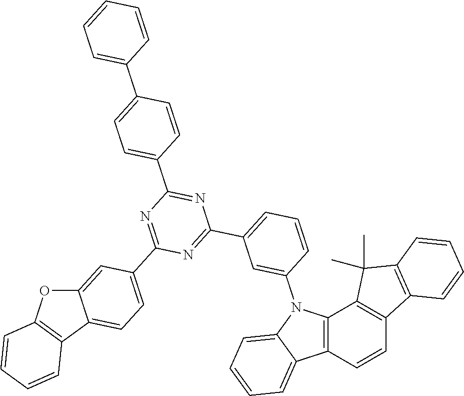 [SMILES: CC1(C)c2ccccc2-c2ccc3c4ccccc4n(-c4cccc(-c5nc(-c6ccc(-c7ccccc7)cc6)nc(-c6ccc7c(c6)oc6ccccc67)n5)c4)c3c21] | 580 |
| 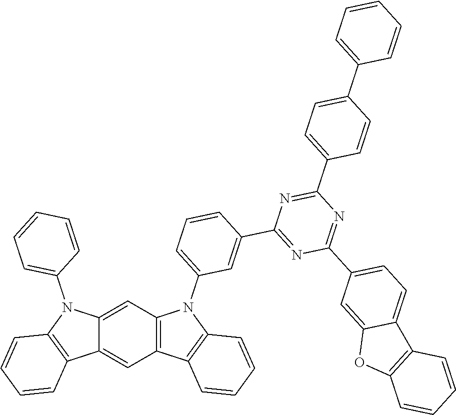 [SMILES: c1ccc(-c2ccc(-c3nc(-c4cccc(-n5c6ccccc6c6cc7c8ccccc8n(-c8ccccc8)c7cc65)c4)nc(-c4ccc5c(c4)oc4ccccc45)n3)cc2)cc1] | 581 |
| 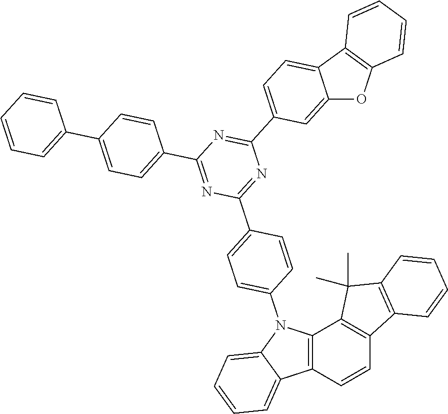 [SMILES: CC1(C)c2ccccc2-c2ccc3c4ccccc4n(-c4ccc(-c5nc(-c6ccc(-c7ccccc7)cc6)nc(-c6ccc7c(c6)oc6ccccc67)n5)cc4)c3c21] | 582 |
| 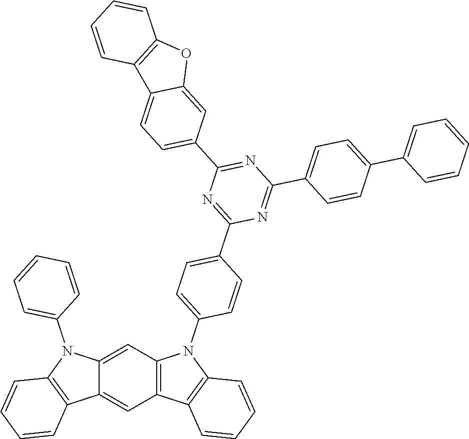 [SMILES: c1ccc(-c2ccc(-c3nc(-c4ccc(-n5c6ccccc6c6cc7c8ccccc8n(-c8ccccc8)c7cc65)cc4)nc(-c4ccc5c(c4)oc4ccccc45)n3)cc2)cc1] | 583 |
| 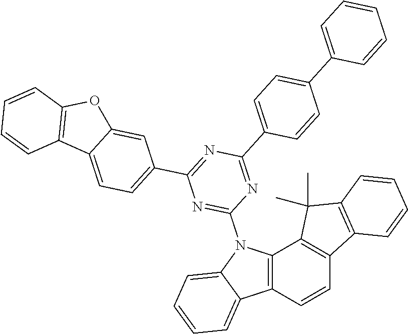 [SMILES: CC1(C)c2ccccc2-c2ccc3c4ccccc4n(-c4nc(-c5ccc(-c6ccccc6)cc5)nc(-c5ccc6c(c5)oc5ccccc56)n4)c3c21] | 584 |
| 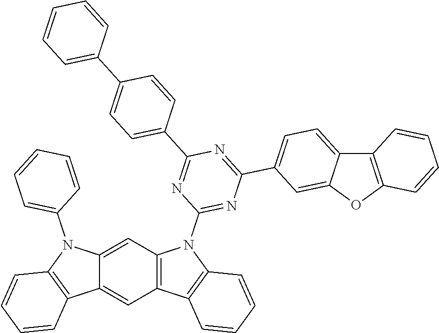 [SMILES: c1ccc(-c2ccc(-c3nc(-c4ccc5c(c4)oc4ccccc45)nc(-n4c5ccccc5c5cc6c7ccccc7n(-c7ccccc7)c6cc54)n3)cc2)cc1] | 585 |
| 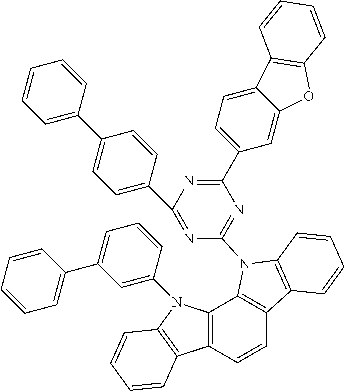 [SMILES: c1ccc(-c2ccc(-c3nc(-c4ccc5c(c4)oc4ccccc45)nc(-n4c5ccccc5c5ccc6c7ccccc7n(-c7cccc(-c8ccccc8)c7)c6c54)n3)cc2)cc1] | 586 |
| 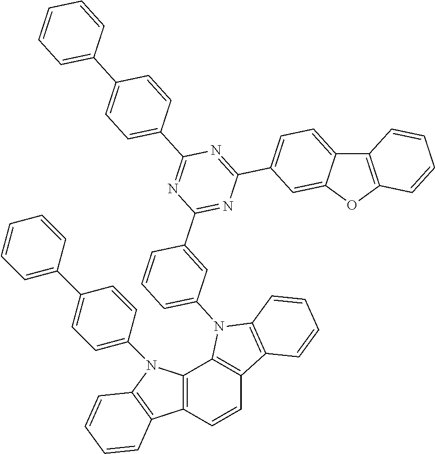 [SMILES: c1ccc(-c2ccc(-c3nc(-c4cccc(-n5c6ccccc6c6ccc7c8ccccc8n(-c8ccc(-c9ccccc9)cc8)c7c65)c4)nc(-c4ccc5c(c4)oc4ccccc45)n3)cc2)cc1] | 587 |
| 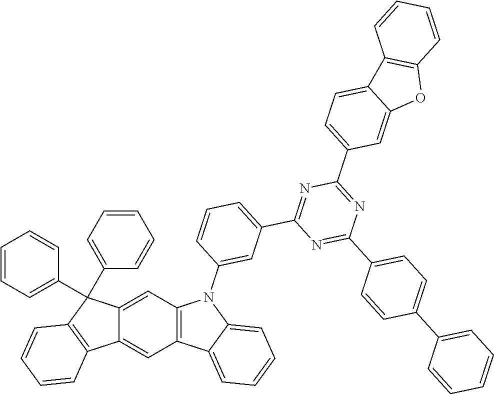 [SMILES: c1ccc(-c2ccc(-c3nc(-c4cccc(-n5c6ccccc6c6cc7c(cc65)C(c5ccccc5)(c5ccccc5)c5ccccc5-7)c4)nc(-c4ccc5c(c4)oc4ccccc45)n3)cc2)cc1] | 588 |
| 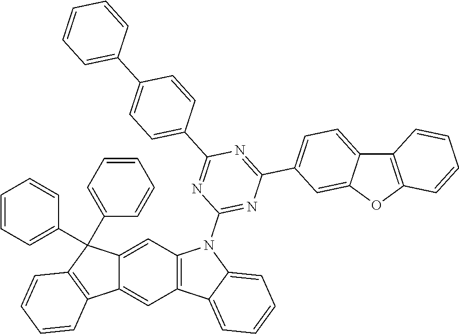 [SMILES: c1ccc(-c2ccc(-c3nc(-c4ccc5c(c4)oc4ccccc45)nc(-n4c5ccccc5c5cc6c(cc54)C(c4ccccc4)(c4ccccc4)c4ccccc4-6)n3)cc2)cc1] | 589 |
| 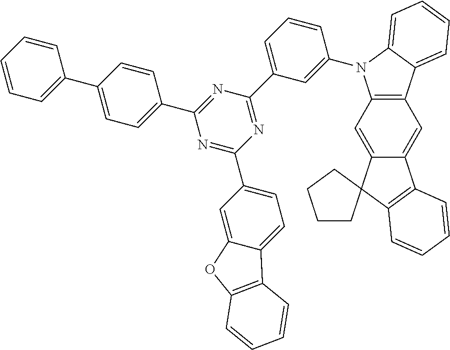 [SMILES: c1ccc(-c2ccc(-c3nc(-c4cccc(-n5c6ccccc6c6cc7c(cc65)C5(CCCC5)c5ccccc5-7)c4)nc(-c4ccc5c(c4)oc4ccccc45)n3)cc2)cc1] | 590 |
| 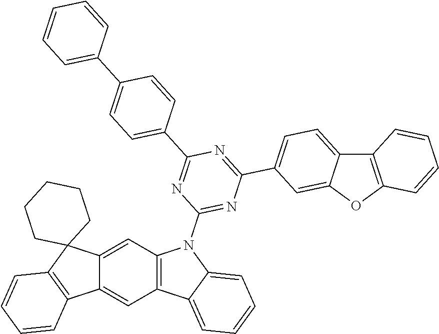 [SMILES: c1ccc(-c2ccc(-c3nc(-c4ccc5c(c4)oc4ccccc45)nc(-n4c5ccccc5c5cc6c(cc54)C4(CCCCC4)c4ccccc4-6)n3)cc2)cc1] | 591 |
| 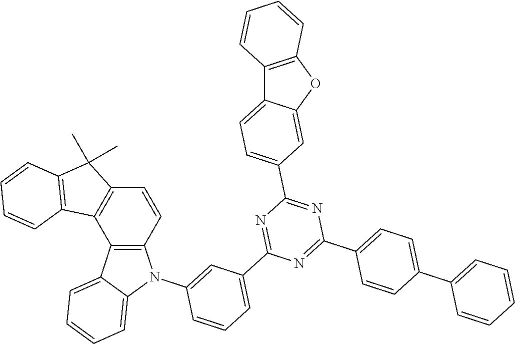 [SMILES: CC1(C)c2ccccc2-c2c1ccc1c2c2ccccc2n1-c1cccc(-c2nc(-c3ccc(-c4ccccc4)cc3)nc(-c3ccc4c(c3)oc3ccccc34)n2)c1] | 592 |
| 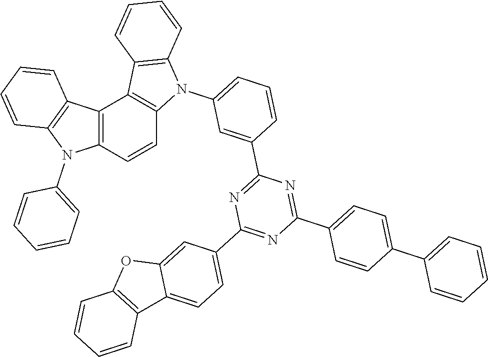 [SMILES: c1ccc(-c2ccc(-c3nc(-c4cccc(-n5c6ccccc6c6c7c8ccccc8n(-c8ccccc8)c7ccc65)c4)nc(-c4ccc5c(c4)oc4ccccc45)n3)cc2)cc1] | 593 |
| 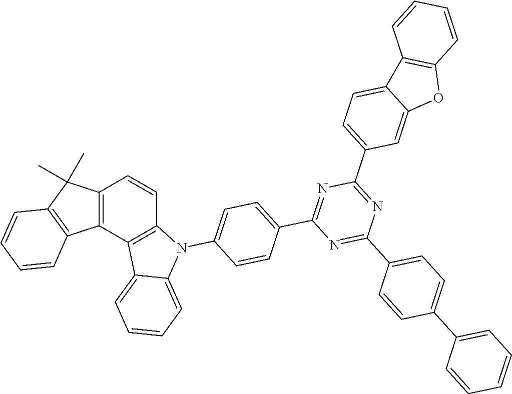 [SMILES: CC1(C)c2ccccc2-c2c1ccc1c2c2ccccc2n1-c1ccc(-c2nc(-c3ccc(-c4ccccc4)cc3)nc(-c3ccc4c(c3)oc3ccccc34)n2)cc1] | 594 |
| 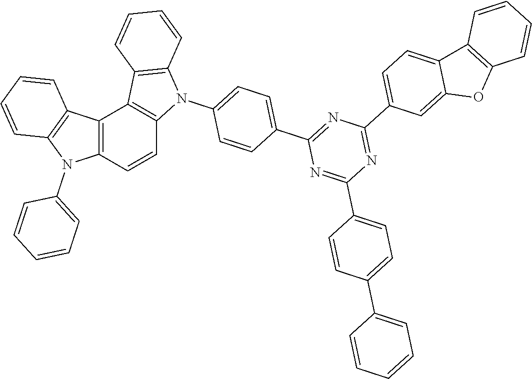 [SMILES: c1ccc(-c2ccc(-c3nc(-c4ccc(-n5c6ccccc6c6c7c8ccccc8n(-c8ccccc8)c7ccc65)cc4)nc(-c4ccc5c(c4)oc4ccccc45)n3)cc2)cc1] | 595 |
| 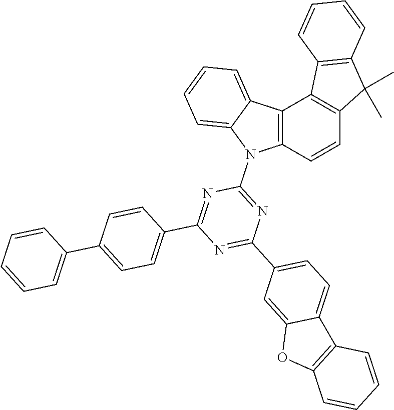 [SMILES: CC1(C)c2ccccc2-c2c1ccc1c2c2ccccc2n1-c1nc(-c2ccc(-c3ccccc3)cc2)nc(-c2ccc3c(c2)oc2ccccc23)n1] | 596 |
| 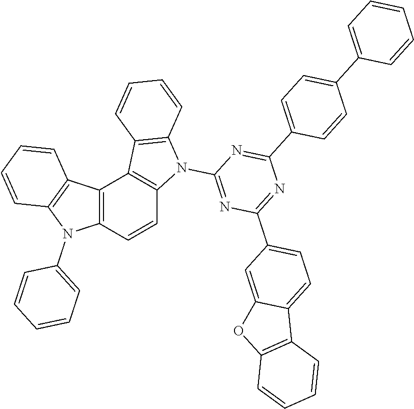 [SMILES: c1ccc(-c2ccc(-c3nc(-c4ccc5c(c4)oc4ccccc45)nc(-n4c5ccccc5c5c6c7ccccc7n(-c7ccccc7)c6ccc54)n3)cc2)cc1] | 597 |
| 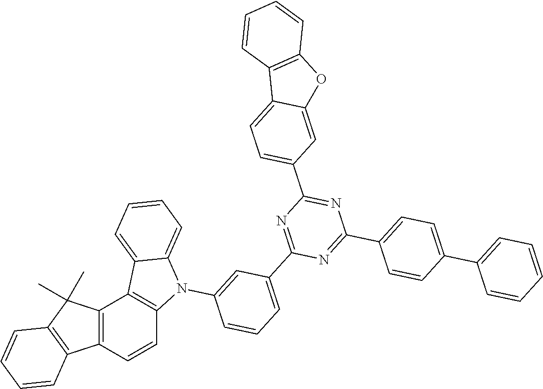 | 598 |
| 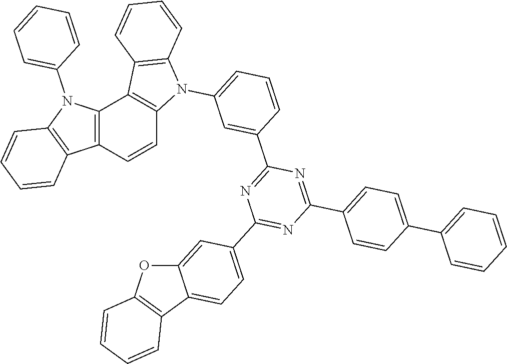 [SMILES: c1ccc(-c2ccc(-c3nc(-c4cccc(-n5c6ccccc6c6c5ccc5c7ccccc7n(-c7ccccc7)c56)c4)nc(-c4ccc5c(c4)oc4ccccc45)n3)cc2)cc1] | 599 |
| 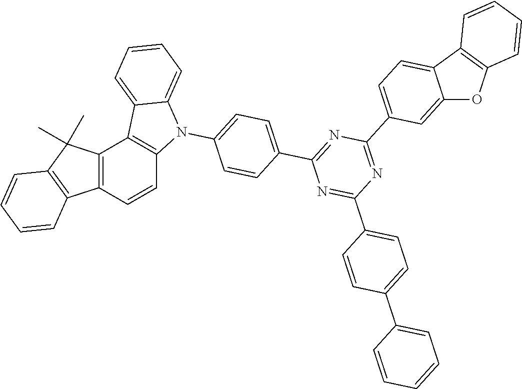 | 600 |
| 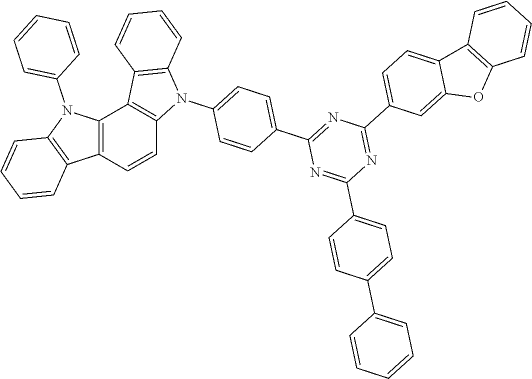 [SMILES: c1ccc(-c2ccc(-c3nc(-c4ccc(-n5c6ccccc6c6c5ccc5c7ccccc7n(-c7ccccc7)c56)cc4)nc(-c4ccc5c(c4)oc4ccccc45)n3)cc2)cc1] | 601 |
| 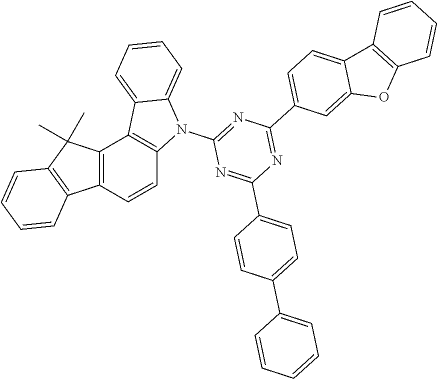 [SMILES: CC1(C)c2ccccc2-c2ccc3c(c21)c1ccccc1n3-c1nc(-c2ccc(-c3ccccc3)cc2)nc(-c2ccc3c(c2)oc2ccccc23)n1] | 602 |
| 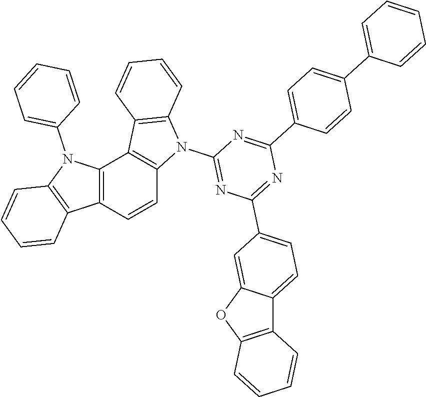 [SMILES: c1ccc(-c2ccc(-c3nc(-c4ccc5c(c4)oc4ccccc45)nc(-n4c5ccccc5c5c4ccc4c6ccccc6n(-c6ccccc6)c45)n3)cc2)cc1] | 603 |
| 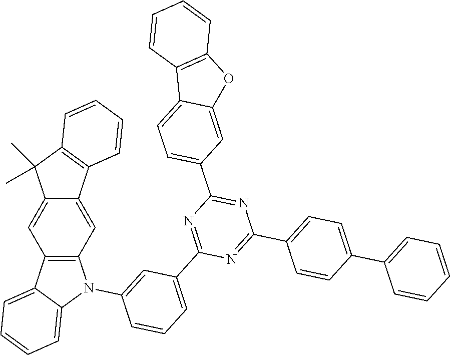 [SMILES: CC1(C)c2ccccc2-c2cc3c(cc21)c1ccccc1n3-c1cccc(-c2nc(-c3ccc(-c4ccccc4)cc3)nc(-c3ccc4c(c3)oc3ccccc34)n2)c1] | 604 |
| 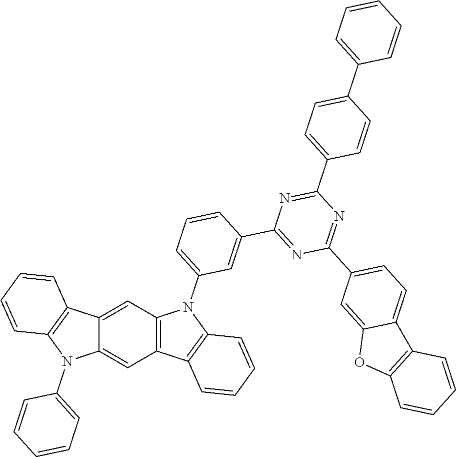 [SMILES: c1ccc(-c2ccc(-c3nc(-c4cccc(-n5c6ccccc6c6cc7c(cc65)c5ccccc5n7-c5ccccc5)c4)nc(-c4ccc5c(c4)oc4ccccc45)n3)cc2)cc1] | 605 |
| 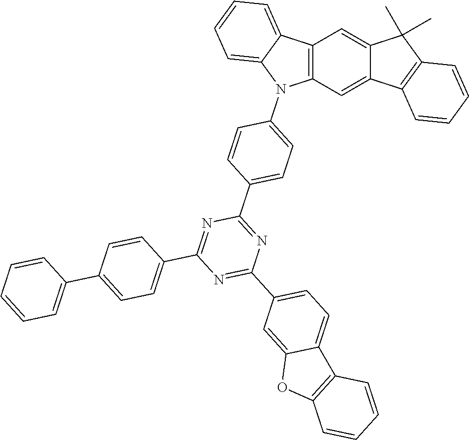 [SMILES: CC1(C)c2ccccc2-c2cc3c(cc21)c1ccccc1n3-c1ccc(-c2nc(-c3ccc(-c4ccccc4)cc3)nc(-c3ccc4c(c3)oc3ccccc34)n2)cc1] | 606 |
| 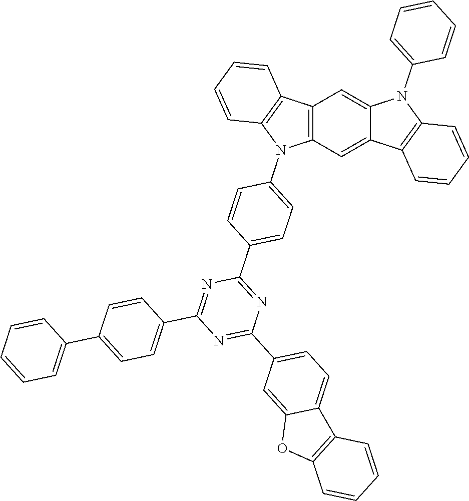 [SMILES: c1ccc(-c2ccc(-c3nc(-c4ccc(-n5c6ccccc6c6cc7c(cc65)c5ccccc5n7-c5ccccc5)cc4)nc(-c4ccc5c(c4)oc4ccccc45)n3)cc2)cc1] | 607 |
| 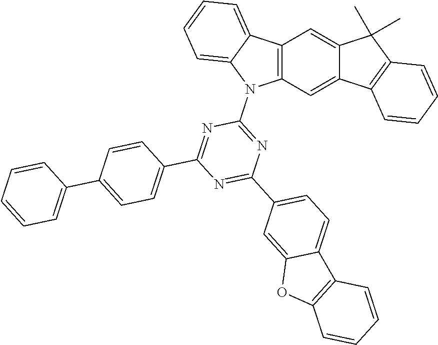 [SMILES: CC1(C)c2ccccc2-c2cc3c(cc21)c1ccccc1n3-c1nc(-c2ccc(-c3ccccc3)cc2)nc(-c2ccc3c(c2)oc2ccccc23)n1] | 608 |
| 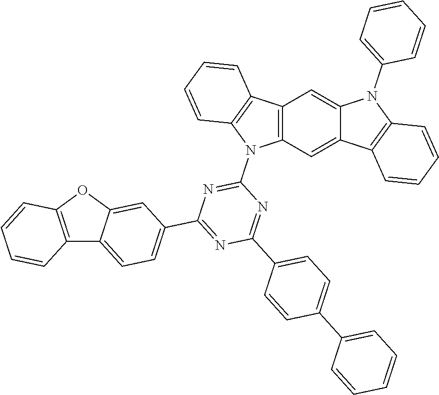 [SMILES: c1ccc(-c2ccc(-c3nc(-c4ccc5c(c4)oc4ccccc45)nc(-n4c5ccccc5c5cc6c(cc54)c4ccccc4n6-c4ccccc4)n3)cc2)cc1] | 609 |
| 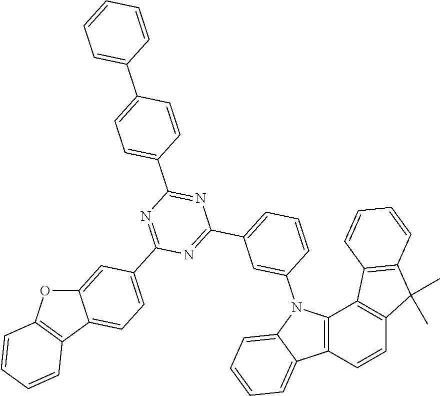 [SMILES: CC1(C)c2ccccc2-c2c1ccc1c3ccccc3n(-c3cccc(-c4nc(-c5ccc(-c6ccccc6)cc5)nc(-c5ccc6c(c5)oc5ccccc56)n4)c3)c21] | 610 |
| 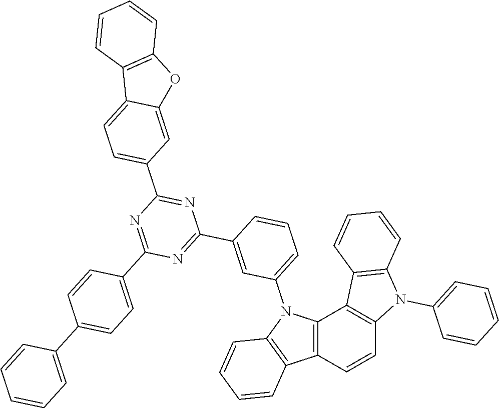 [SMILES: c1ccc(-c2ccc(-c3nc(-c4cccc(-n5c6ccccc6c6ccc7c(c8ccccc8n7-c7ccccc7)c65)c4)nc(-c4ccc5c(c4)oc4ccccc45)n3)cc2)cc1] | 611 |
| 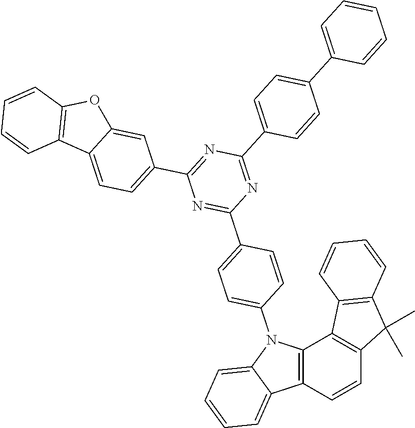 [SMILES: CC1(C)c2ccccc2-c2c1ccc1c3ccccc3n(-c3ccc(-c4nc(-c5ccc(-c6ccccc6)cc5)nc(-c5ccc6c(c5)oc5ccccc56)n4)cc3)c21] | 612 |
| 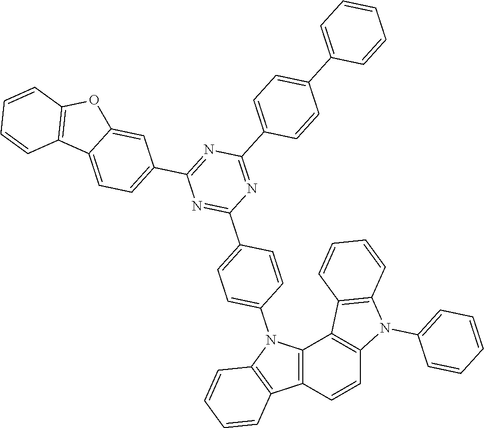 [SMILES: c1ccc(-c2ccc(-c3nc(-c4ccc(-n5c6ccccc6c6ccc7c(c8ccccc8n7-c7ccccc7)c65)cc4)nc(-c4ccc5c(c4)oc4ccccc45)n3)cc2)cc1] | 613 |
| 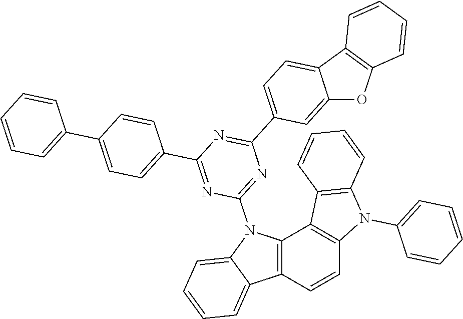 [SMILES: c1ccc(-c2ccc(-c3nc(-c4ccc5c(c4)oc4ccccc45)nc(-n4c5ccccc5c5ccc6c(c7ccccc7n6-c6ccccc6)c54)n3)cc2)cc1] | 614 |
| 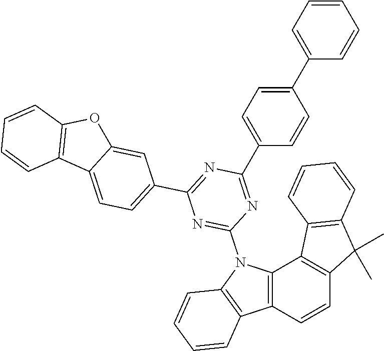 [SMILES: CC1(C)c2ccccc2-c2c1ccc1c3ccccc3n(-c3nc(-c4ccc(-c5ccccc5)cc4)nc(-c4ccc5c(c4)oc4ccccc45)n3)c21] | 615 |
| 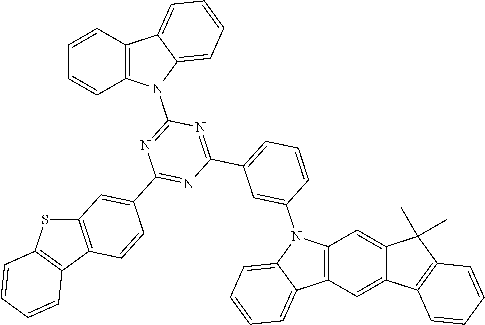 [SMILES: CC1(C)c2ccccc2-c2cc3c4ccccc4n(-c4cccc(-c5nc(-c6ccc7c(c6)sc6ccccc67)nc(-n6c7ccccc7c7ccccc76)n5)c4)c3cc21] | 616 |
| 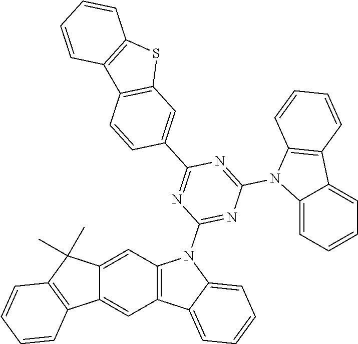 [SMILES: CC1(C)c2ccccc2-c2cc3c4ccccc4n(-c4nc(-c5ccc6c(c5)sc5ccccc56)nc(-n5c6ccccc6c6ccccc65)n4)c3cc21] | 617 |
| 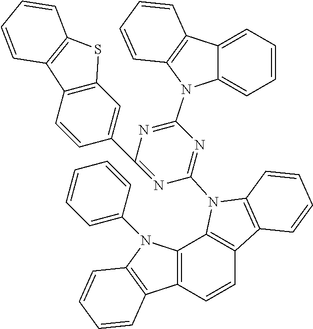 [SMILES: c1ccc(-n2c3ccccc3c3ccc4c5ccccc5n(-c5nc(-c6ccc7c(c6)sc6ccccc67)nc(-n6c7ccccc7c7ccccc76)n5)c4c32)cc1] | 618 |
| 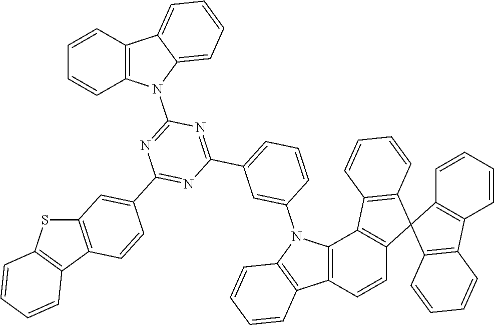 [SMILES: c1cc(-c2nc(-c3ccc4c(c3)sc3ccccc34)nc(-n3c4ccccc4c4ccccc43)n2)cc(-n2c3ccccc3c3ccc4c(c32)-c2ccccc2C42c3ccccc3-c3ccccc32)c1] | 619 |
| 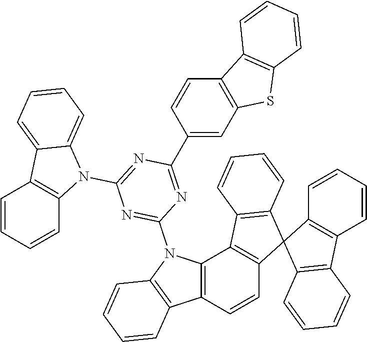 [SMILES: c1ccc2c(c1)-c1ccccc1C21c2ccccc2-c2c1ccc1c3ccccc3n(-c3nc(-c4ccc5c(c4)sc4ccccc45)nc(-n4c5ccccc5c5ccccc54)n3)c21] | 620 |
| 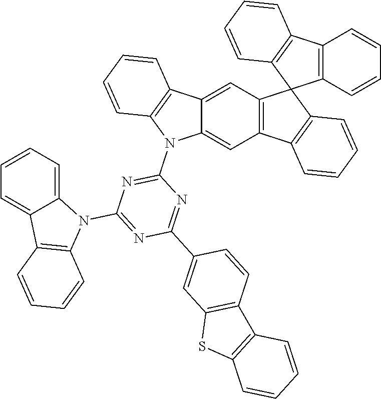 [SMILES: c1ccc2c(c1)-c1ccccc1C21c2ccccc2-c2cc3c(cc21)c1ccccc1n3-c1nc(-c2ccc3c(c2)sc2ccccc23)nc(-n2c3ccccc3c3ccccc32)n1] | 621 |
| 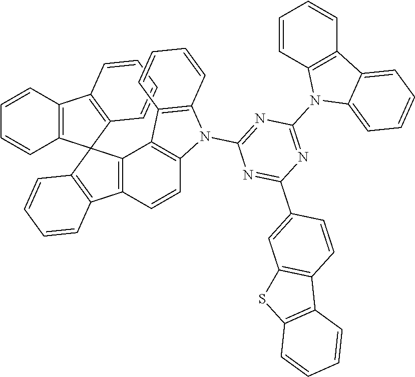 [SMILES: c1ccc2c(c1)-c1ccccc1C21c2ccccc2-c2ccc3c(c21)c1ccccc1n3-c1nc(-c2ccc3c(c2)sc2ccccc23)nc(-n2c3ccccc3c3ccccc32)n1] | 622 |
| 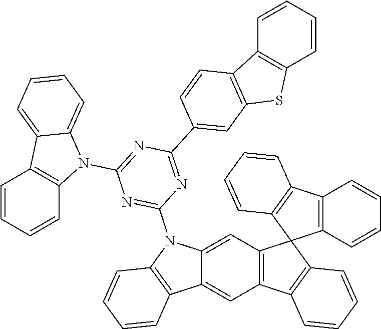 [SMILES: c1ccc2c(c1)-c1ccccc1C21c2ccccc2-c2cc3c4ccccc4n(-c4nc(-c5ccc6c(c5)sc5ccccc56)nc(-n5c6ccccc6c6ccccc65)n4)c3cc21] | 623 |
| 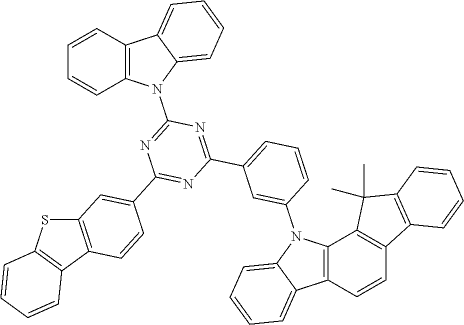 [SMILES: CC1(C)c2ccccc2-c2ccc3c4ccccc4n(-c4cccc(-c5nc(-c6ccc7c(c6)sc6ccccc67)nc(-n6c7ccccc7c7ccccc76)n5)c4)c3c21] | 624 |
| 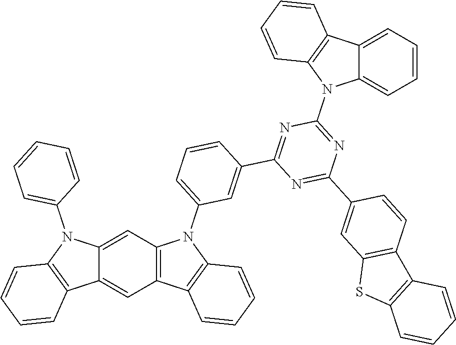 [SMILES: c1ccc(-n2c3ccccc3c3cc4c5ccccc5n(-c5cccc(-c6nc(-c7ccc8c(c7)sc7ccccc78)nc(-n7c8ccccc8c8ccccc87)n6)c5)c4cc32)cc1] | 625 |
| 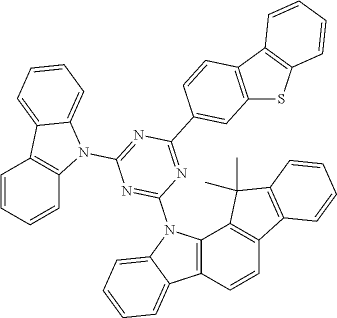 [SMILES: CC1(C)c2ccccc2-c2ccc3c4ccccc4n(-c4nc(-c5ccc6c(c5)sc5ccccc56)nc(-n5c6ccccc6c6ccccc65)n4)c3c21] | 626 |
| 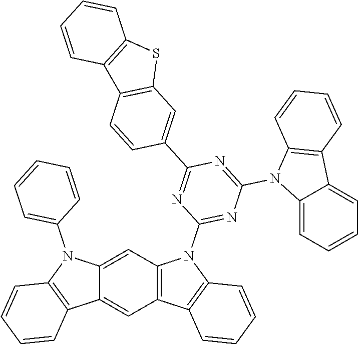 [SMILES: c1ccc(-n2c3ccccc3c3cc4c5ccccc5n(-c5nc(-c6ccc7c(c6)sc6ccccc67)nc(-n6c7ccccc7c7ccccc76)n5)c4cc32)cc1] | 627 |
| 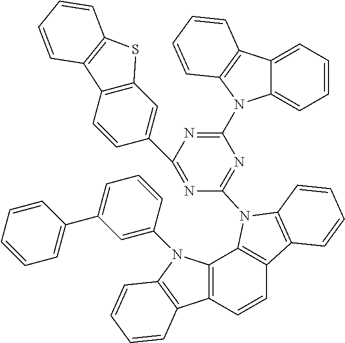 [SMILES: c1ccc(-c2cccc(-n3c4ccccc4c4ccc5c6ccccc6n(-c6nc(-c7ccc8c(c7)sc7ccccc78)nc(-n7c8ccccc8c8ccccc87)n6)c5c43)c2)cc1] | 628 |
| 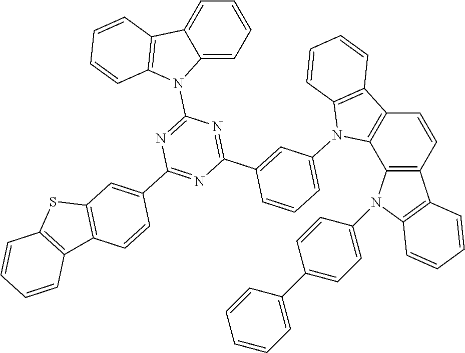 [SMILES: c1ccc(-c2ccc(-n3c4ccccc4c4ccc5c6ccccc6n(-c6cccc(-c7nc(-c8ccc9c(c8)sc8ccccc89)nc(-n8c9ccccc9c9ccccc98)n7)c6)c5c43)cc2)cc1] | 629 |
| 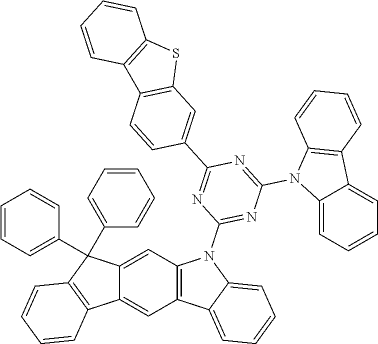 [SMILES: c1ccc(C2(c3ccccc3)c3ccccc3-c3cc4c5ccccc5n(-c5nc(-c6ccc7c(c6)sc6ccccc67)nc(-n6c7ccccc7c7ccccc76)n5)c4cc32)cc1] | 630 |
| 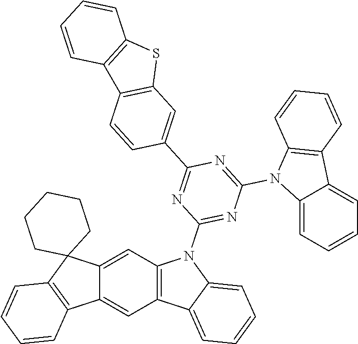 [SMILES: c1ccc2c(c1)-c1cc3c4ccccc4n(-c4nc(-c5ccc6c(c5)sc5ccccc56)nc(-n5c6ccccc6c6ccccc65)n4)c3cc1C21CCCCC1] | 631 |
| 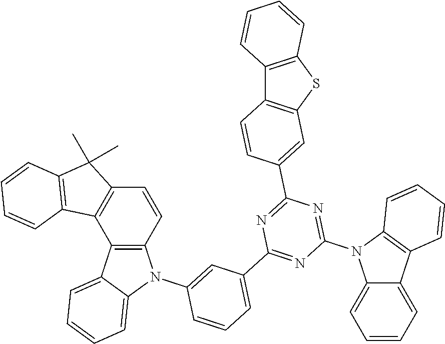 [SMILES: CC1(C)c2ccccc2-c2c1ccc1c2c2ccccc2n1-c1cccc(-c2nc(-c3ccc4c(c3)sc3ccccc34)nc(-n3c4ccccc4c4ccccc43)n2)c1] | 632 |
| 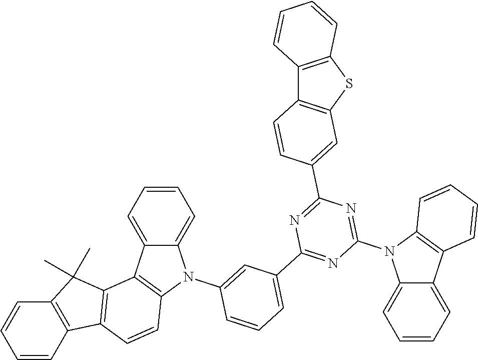 [SMILES: CC1(C)c2ccccc2-c2ccc3c(c21)c1ccccc1n3-c1cccc(-c2nc(-c3ccc4c(c3)sc3ccccc34)nc(-n3c4ccccc4c4ccccc43)n2)c1] | 633 |
| 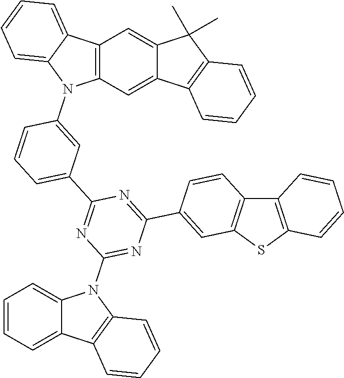 [SMILES: CC1(C)c2ccccc2-c2cc3c(cc21)c1ccccc1n3-c1cccc(-c2nc(-c3ccc4c(c3)sc3ccccc34)nc(-n3c4ccccc4c4ccccc43)n2)c1] | 634 |
| 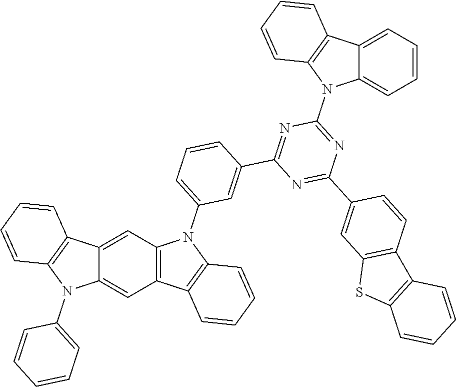 [SMILES: c1ccc(-n2c3ccccc3c3cc4c(cc32)c2ccccc2n4-c2cccc(-c3nc(-c4ccc5c(c4)sc4ccccc45)nc(-n4c5ccccc5c5ccccc54)n3)c2)cc1] | 635 |
| 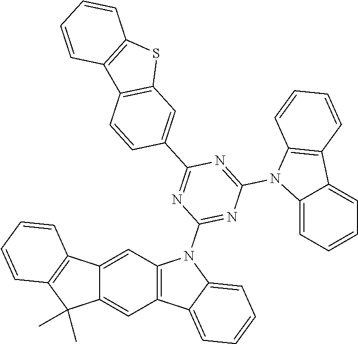 [SMILES: CC1(C)c2ccccc2-c2cc3c(cc21)c1ccccc1n3-c1nc(-c2ccc3c(c2)sc2ccccc23)nc(-n2c3ccccc3c3ccccc32)n1] | 636 |
| 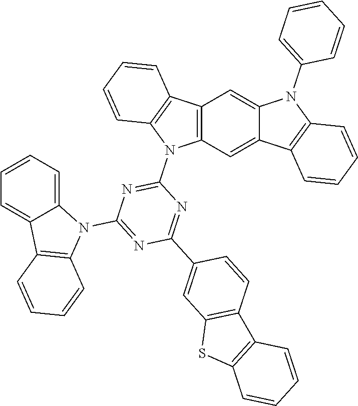 [SMILES: c1ccc(-n2c3ccccc3c3cc4c(cc32)c2ccccc2n4-c2nc(-c3ccc4c(c3)sc3ccccc34)nc(-n3c4ccccc4c4ccccc43)n2)cc1] | 637 |
| 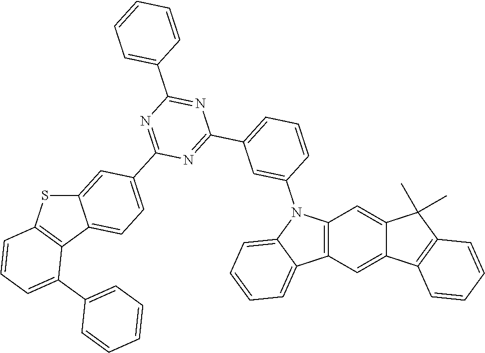 [SMILES: CC1(C)c2ccccc2-c2cc3c4ccccc4n(-c4cccc(-c5nc(-c6ccccc6)nc(-c6ccc7c(c6)sc6cccc(-c8ccccc8)c67)n5)c4)c3cc21] | 638 |
| 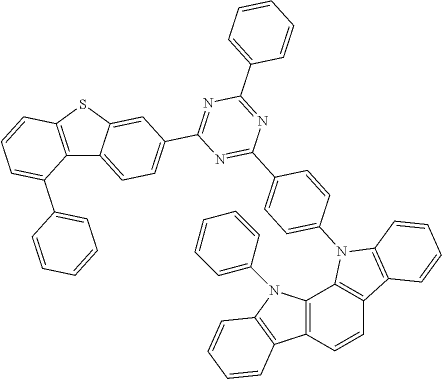 [SMILES: c1ccc(-c2nc(-c3ccc(-n4c5ccccc5c5ccc6c7ccccc7n(-c7ccccc7)c6c54)cc3)nc(-c3ccc4c(c3)sc3cccc(-c5ccccc5)c34)n2)cc1] | 639 |
| 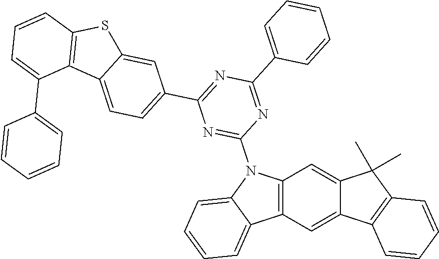 [SMILES: CC1(C)c2ccccc2-c2cc3c4ccccc4n(-c4nc(-c5ccccc5)nc(-c5ccc6c(c5)sc5cccc(-c7ccccc7)c56)n4)c3cc21] | 640 |
| 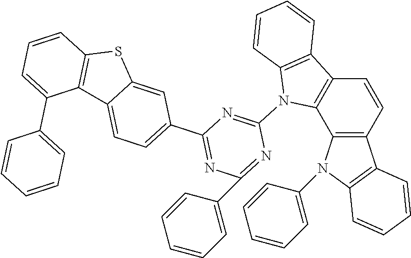 [SMILES: c1ccc(-c2nc(-c3ccc4c(c3)sc3cccc(-c5ccccc5)c34)nc(-n3c4ccccc4c4ccc5c6ccccc6n(-c6ccccc6)c5c43)n2)cc1] | 641 |
| 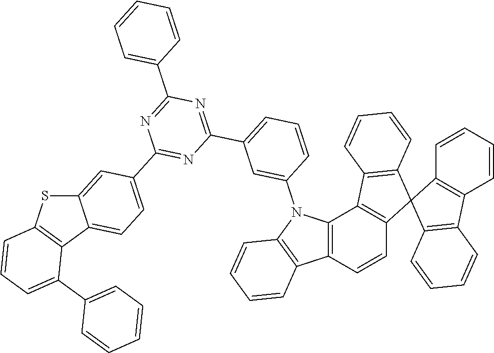 [SMILES: c1ccc(-c2nc(-c3cccc(-n4c5ccccc5c5ccc6c(c54)-c4ccccc4C64c5ccccc5-c5ccccc54)c3)nc(-c3ccc4c(c3)sc3cccc(-c5ccccc5)c34)n2)cc1] | 642 |
| 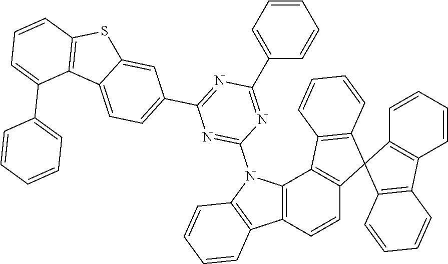 [SMILES: c1ccc(-c2nc(-c3ccc4c(c3)sc3cccc(-c5ccccc5)c34)nc(-n3c4ccccc4c4ccc5c(c43)-c3ccccc3C53c4ccccc4-c4ccccc43)n2)cc1] | 643 |
| 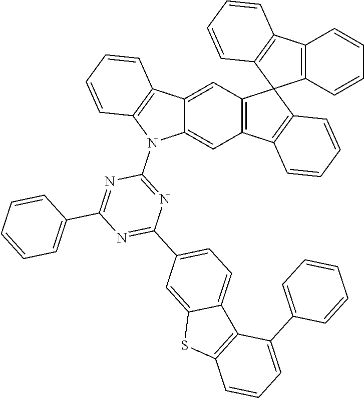 [SMILES: c1ccc(-c2nc(-c3ccc4c(c3)sc3cccc(-c5ccccc5)c34)nc(-n3c4ccccc4c4cc5c(cc43)-c3ccccc3C53c4ccccc4-c4ccccc43)n2)cc1] | 644 |
| 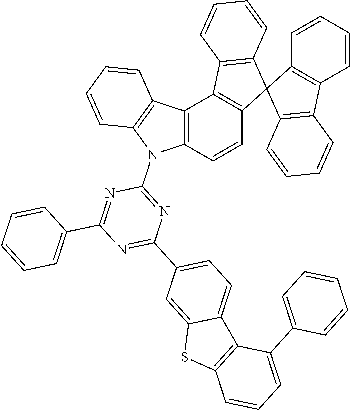 [SMILES: c1ccc(-c2nc(-c3ccc4c(c3)sc3cccc(-c5ccccc5)c34)nc(-n3c4ccccc4c4c5c(ccc43)C3(c4ccccc4-c4ccccc43)c3ccccc3-5)n2)cc1] | 645 |
| 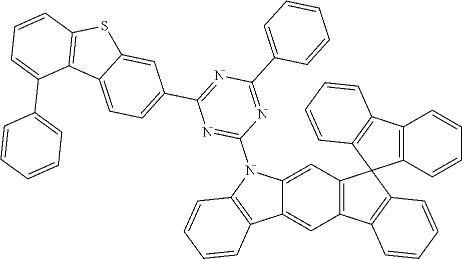 [SMILES: c1ccc(-c2nc(-c3ccc4c(c3)sc3cccc(-c5ccccc5)c34)nc(-n3c4ccccc4c4cc5c(cc43)C3(c4ccccc4-c4ccccc43)c3ccccc3-5)n2)cc1] | 646 |
| 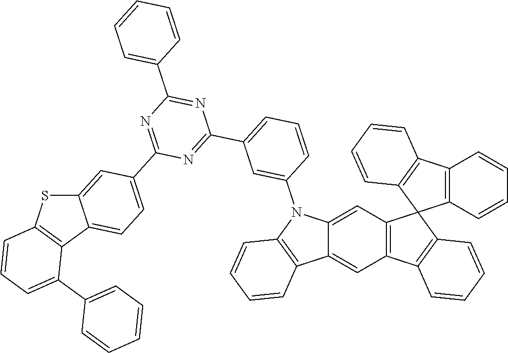 [SMILES: c1ccc(-c2nc(-c3cccc(-n4c5ccccc5c5cc6c(cc54)C4(c5ccccc5-c5ccccc54)c4ccccc4-6)c3)nc(-c3ccc4c(c3)sc3cccc(-c5ccccc5)c34)n2)cc1] | 647 |
| 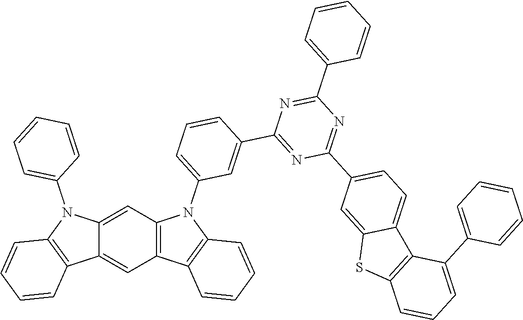 [SMILES: c1ccc(-c2nc(-c3cccc(-n4c5ccccc5c5cc6c7ccccc7n(-c7ccccc7)c6cc54)c3)nc(-c3ccc4c(c3)sc3cccc(-c5ccccc5)c34)n2)cc1] | 648 |
| 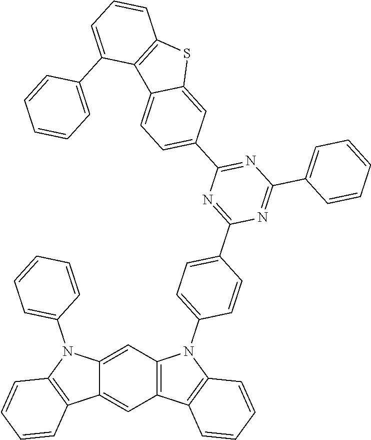 [SMILES: c1ccc(-c2nc(-c3ccc(-n4c5ccccc5c5cc6c7ccccc7n(-c7ccccc7)c6cc54)cc3)nc(-c3ccc4c(c3)sc3cccc(-c5ccccc5)c34)n2)cc1] | 649 |
| 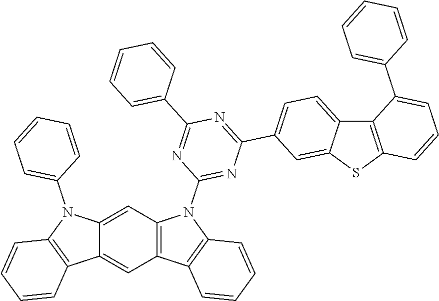 [SMILES: c1ccc(-c2nc(-c3ccc4c(c3)sc3cccc(-c5ccccc5)c34)nc(-n3c4ccccc4c4cc5c6ccccc6n(-c6ccccc6)c5cc43)n2)cc1] | 650 |
| 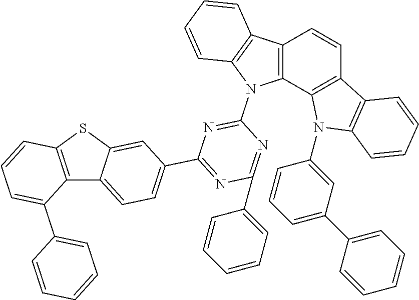 [SMILES: c1ccc(-c2cccc(-n3c4ccccc4c4ccc5c6ccccc6n(-c6nc(-c7ccccc7)nc(-c7ccc8c(c7)sc7cccc(-c9ccccc9)c78)n6)c5c43)c2)cc1] | 651 |
| 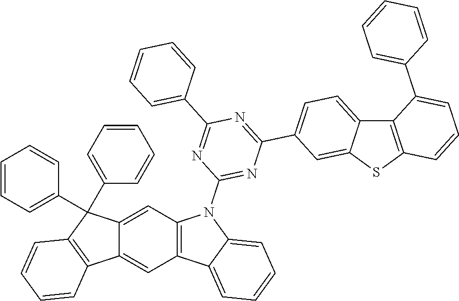 [SMILES: c1ccc(-c2nc(-c3ccc4c(c3)sc3cccc(-c5ccccc5)c34)nc(-n3c4ccccc4c4cc5c(cc43)C(c3ccccc3)(c3ccccc3)c3ccccc3-5)n2)cc1] | 652 |
| 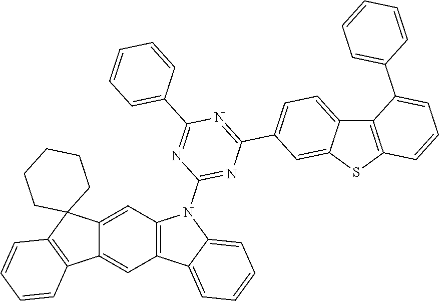 [SMILES: c1ccc(-c2nc(-c3ccc4c(c3)sc3cccc(-c5ccccc5)c34)nc(-n3c4ccccc4c4cc5c(cc43)C3(CCCCC3)c3ccccc3-5)n2)cc1] | 653 |
| 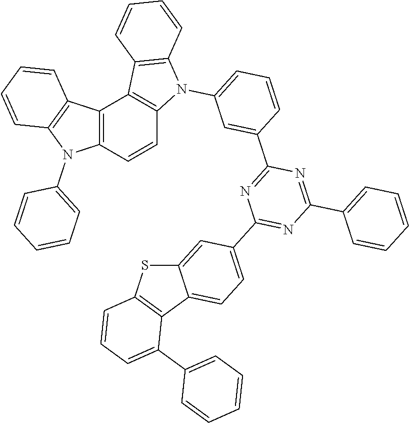 [SMILES: c1ccc(-c2nc(-c3cccc(-n4c5ccccc5c5c6c7ccccc7n(-c7ccccc7)c6ccc54)c3)nc(-c3ccc4c(c3)sc3cccc(-c5ccccc5)c34)n2)cc1] | 654 |
| 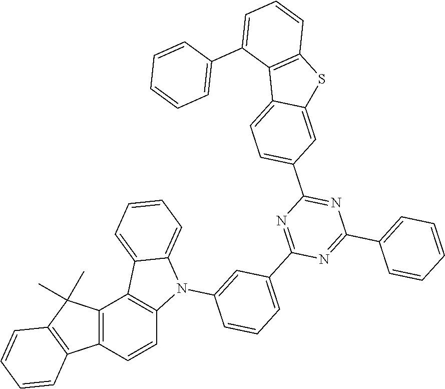 [SMILES: CC1(C)c2ccccc2-c2ccc3c(c21)c1ccccc1n3-c1cccc(-c2nc(-c3ccccc3)nc(-c3ccc4c(c3)sc3cccc(-c5ccccc5)c34)n2)c1] | 655 |
| 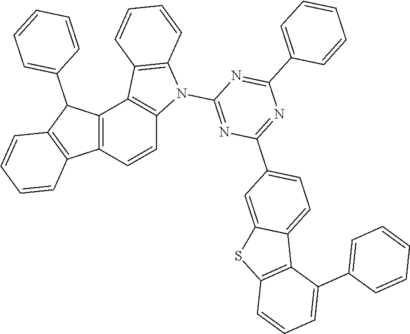 [SMILES: c1ccc(-c2nc(-c3ccc4c(c3)sc3cccc(-c5ccccc5)c34)nc(-n3c4ccccc4c4c5c(ccc43)-c3ccccc3C5c3ccccc3)n2)cc1] | 656 |
| 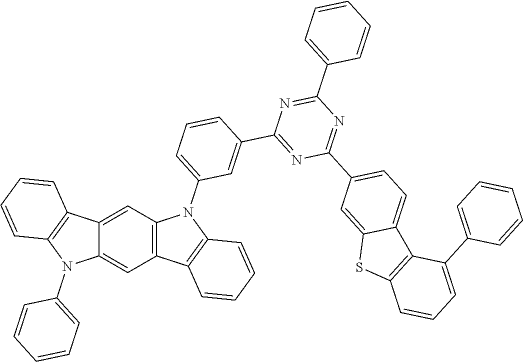 [SMILES: c1ccc(-c2nc(-c3cccc(-n4c5ccccc5c5cc6c(cc54)c4ccccc4n6-c4ccccc4)c3)nc(-c3ccc4c(c3)sc3cccc(-c5ccccc5)c34)n2)cc1] | 657 |
| 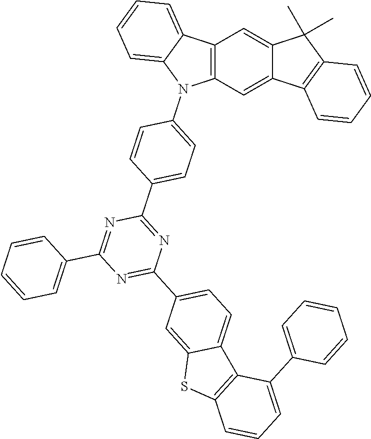 [SMILES: CC1(C)c2ccccc2-c2cc3c(cc21)c1ccccc1n3-c1ccc(-c2nc(-c3ccccc3)nc(-c3ccc4c(c3)sc3cccc(-c5ccccc5)c34)n2)cc1] | 658 |
| 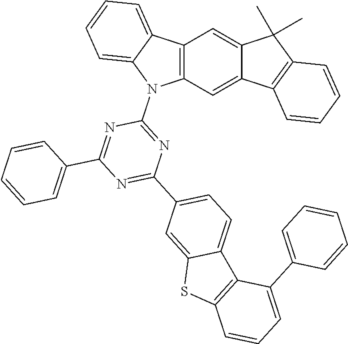 [SMILES: CC1(C)c2ccccc2-c2cc3c(cc21)c1ccccc1n3-c1nc(-c2ccccc2)nc(-c2ccc3c(c2)sc2cccc(-c4ccccc4)c23)n1] | 659 |
| 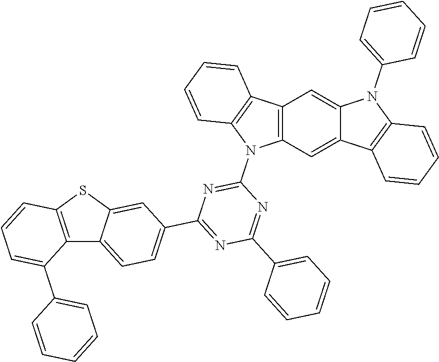 [SMILES: c1ccc(-c2nc(-c3ccc4c(c3)sc3cccc(-c5ccccc5)c34)nc(-n3c4ccccc4c4cc5c(cc43)c3ccccc3n5-c3ccccc3)n2)cc1] | 660 |
| 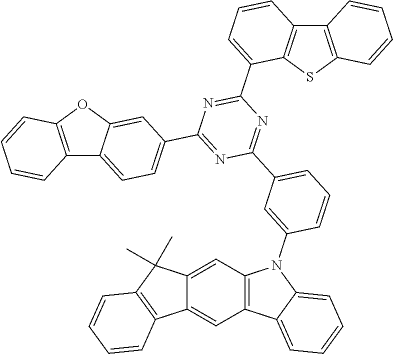 [SMILES: CC1(C)c2ccccc2-c2cc3c4ccccc4n(-c4cccc(-c5nc(-c6ccc7c(c6)oc6ccccc67)nc(-c6cccc7c6sc6ccccc67)n5)c4)c3cc21] | 661 |
| 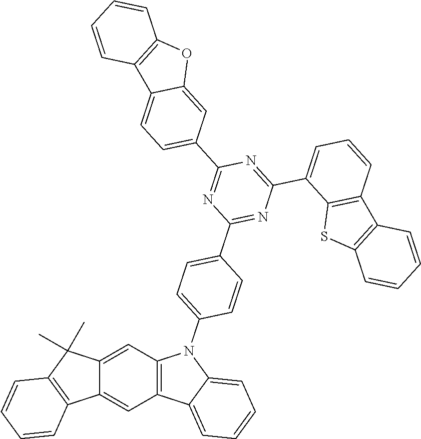 [SMILES: CC1(C)c2ccccc2-c2cc3c4ccccc4n(-c4ccc(-c5nc(-c6ccc7c(c6)oc6ccccc67)nc(-c6cccc7c6sc6ccccc67)n5)cc4)c3cc21] | 662 |
| 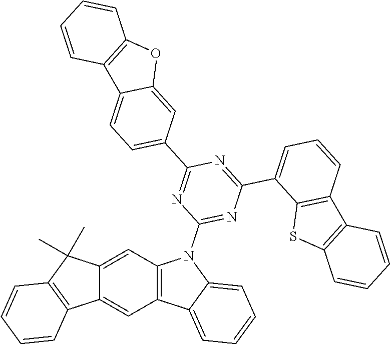 [SMILES: CC1(C)c2ccccc2-c2cc3c4ccccc4n(-c4nc(-c5ccc6c(c5)oc5ccccc56)nc(-c5cccc6c5sc5ccccc56)n4)c3cc21] | 663 |
| 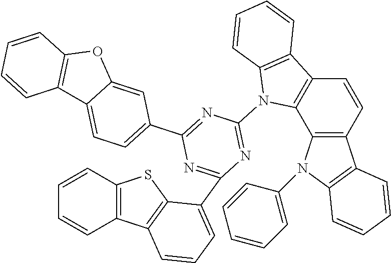 [SMILES: c1ccc(-n2c3ccccc3c3ccc4c5ccccc5n(-c5nc(-c6ccc7c(c6)oc6ccccc67)nc(-c6cccc7c6sc6ccccc67)n5)c4c32)cc1] | 664 |
| 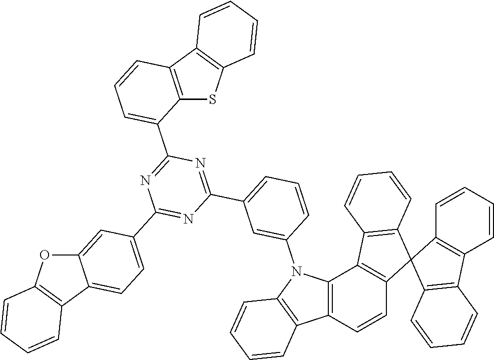 [SMILES: c1cc(-c2nc(-c3ccc4c(c3)oc3ccccc34)nc(-c3cccc4c3sc3ccccc34)n2)cc(-n2c3ccccc3c3ccc4c(c32)-c2ccccc2C42c3ccccc3-c3ccccc32)c1] | 665 |
| 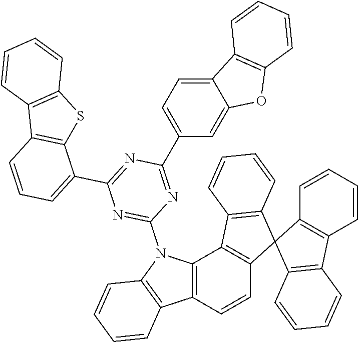 [SMILES: c1ccc2c(c1)-c1ccccc1C21c2ccccc2-c2c1ccc1c3ccccc3n(-c3nc(-c4ccc5c(c4)oc4ccccc45)nc(-c4cccc5c4sc4ccccc45)n3)c21] | 666 |
| 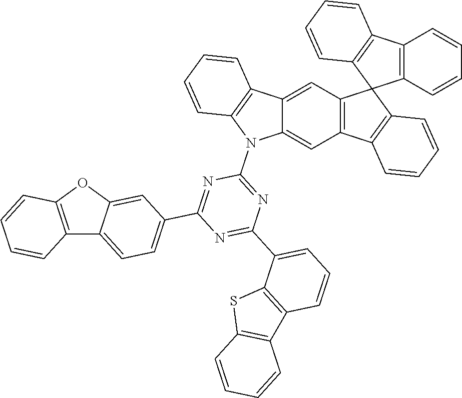 [SMILES: c1ccc2c(c1)-c1ccccc1C21c2ccccc2-c2cc3c(cc21)c1ccccc1n3-c1nc(-c2ccc3c(c2)oc2ccccc23)nc(-c2cccc3c2sc2ccccc23)n1] | 667 |
| 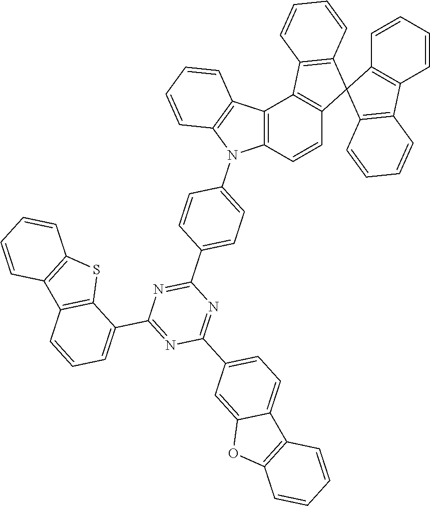 [SMILES: c1ccc2c(c1)-c1ccccc1C21c2ccccc2-c2c1ccc1c2c2ccccc2n1-c1ccc(-c2nc(-c3ccc4c(c3)oc3ccccc34)nc(-c3cccc4c3sc3ccccc34)n2)cc1] | 668 |
| 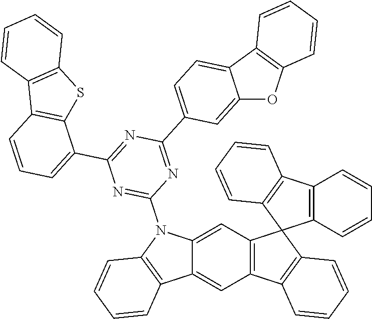 [SMILES: c1ccc2c(c1)-c1ccccc1C21c2ccccc2-c2cc3c4ccccc4n(-c4nc(-c5ccc6c(c5)oc5ccccc56)nc(-c5cccc6c5sc5ccccc56)n4)c3cc21] | 669 |
| 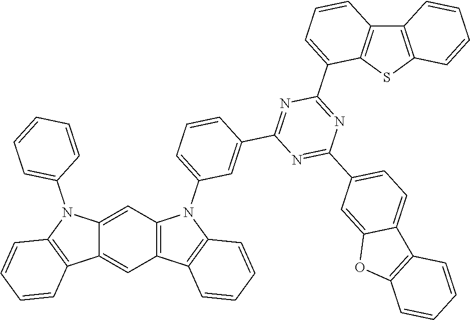 [SMILES: c1ccc(-n2c3ccccc3c3cc4c5ccccc5n(-c5cccc(-c6nc(-c7ccc8c(c7)oc7ccccc78)nc(-c7cccc8c7sc7ccccc78)n6)c5)c4cc32)cc1] | 670 |
| 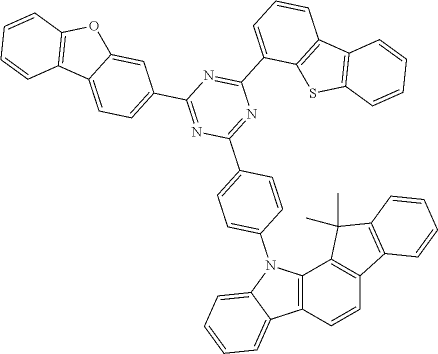 [SMILES: CC1(C)c2ccccc2-c2ccc3c4ccccc4n(-c4ccc(-c5nc(-c6ccc7c(c6)oc6ccccc67)nc(-c6cccc7c6sc6ccccc67)n5)cc4)c3c21] | 671 |
| 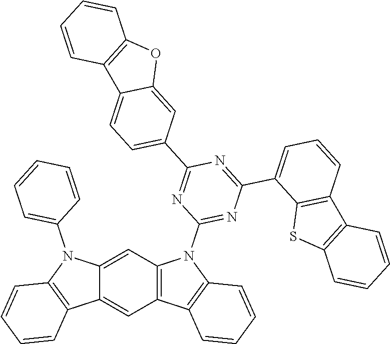 [SMILES: c1ccc(-n2c3ccccc3c3cc4c5ccccc5n(-c5nc(-c6ccc7c(c6)oc6ccccc67)nc(-c6cccc7c6sc6ccccc67)n5)c4cc32)cc1] | 672 |
| 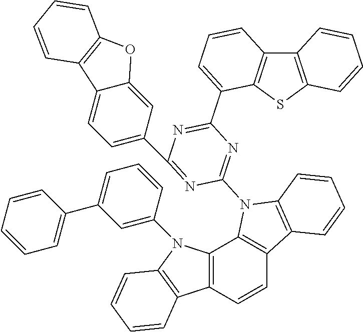 [SMILES: c1ccc(-c2cccc(-n3c4ccccc4c4ccc5c6ccccc6n(-c6nc(-c7ccc8c(c7)oc7ccccc78)nc(-c7cccc8c7sc7ccccc78)n6)c5c43)c2)cc1] | 673 |
| 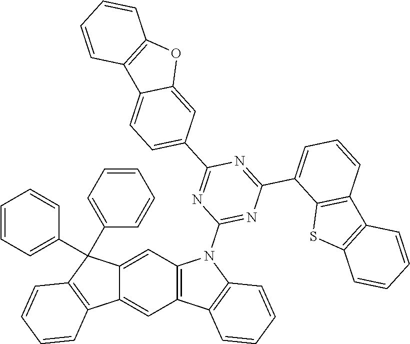 [SMILES: c1ccc(C2(c3ccccc3)c3ccccc3-c3cc4c5ccccc5n(-c5nc(-c6ccc7c(c6)oc6ccccc67)nc(-c6cccc7c6sc6ccccc67)n5)c4cc32)cc1] | 674 |
| 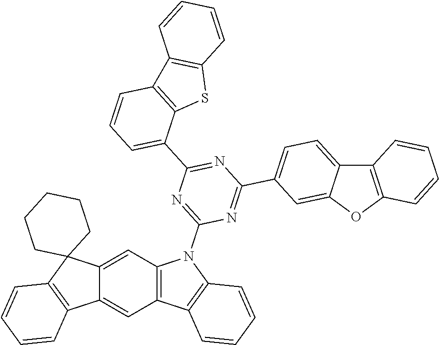 [SMILES: c1ccc2c(c1)-c1cc3c4ccccc4n(-c4nc(-c5ccc6c(c5)oc5ccccc56)nc(-c5cccc6c5sc5ccccc56)n4)c3cc1C21CCCCC1] | 675 |
| 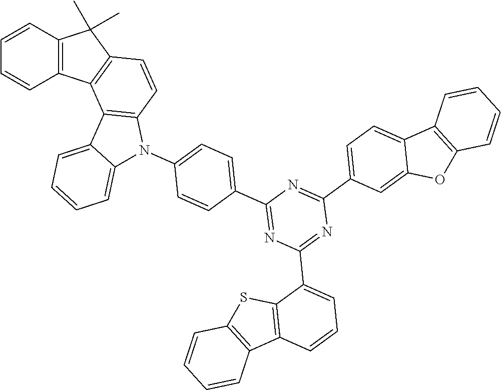 [SMILES: CC1(C)c2ccccc2-c2c1ccc1c2c2ccccc2n1-c1ccc(-c2nc(-c3ccc4c(c3)oc3ccccc34)nc(-c3cccc4c3sc3ccccc34)n2)cc1] | 676 |
| 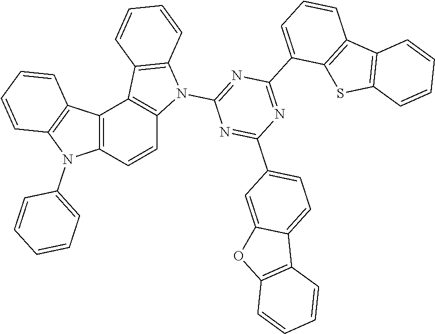 [SMILES: c1ccc(-n2c3ccccc3c3c4c5ccccc5n(-c5nc(-c6ccc7c(c6)oc6ccccc67)nc(-c6cccc7c6sc6ccccc67)n5)c4ccc32)cc1] | 677 |
| 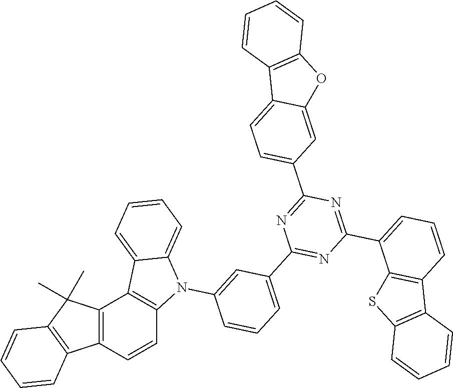 [SMILES: CC1(C)c2ccccc2-c2ccc3c(c21)c1ccccc1n3-c1cccc(-c2nc(-c3ccc4c(c3)oc3ccccc34)nc(-c3cccc4c3sc3ccccc34)n2)c1] | 678 |
| 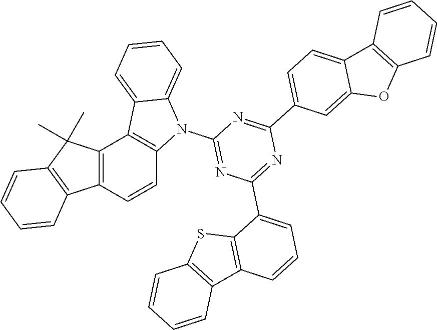 [SMILES: CC1(C)c2ccccc2-c2ccc3c(c21)c1ccccc1n3-c1nc(-c2ccc3c(c2)oc2ccccc23)nc(-c2cccc3c2sc2ccccc23)n1] | 679 |
| 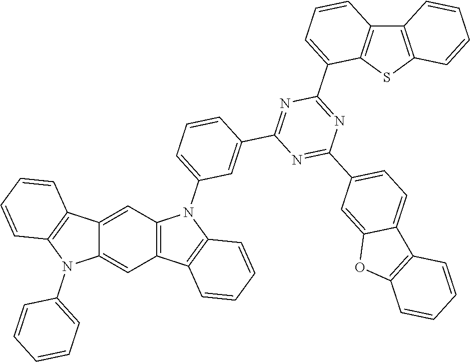 [SMILES: c1ccc(-n2c3ccccc3c3cc4c(cc32)c2ccccc2n4-c2cccc(-c3nc(-c4ccc5c(c4)oc4ccccc45)nc(-c4cccc5c4sc4ccccc45)n3)c2)cc1] | 680 |
| 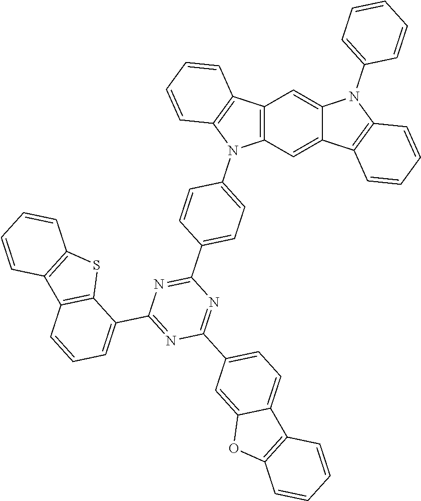 [SMILES: c1ccc(-n2c3ccccc3c3cc4c(cc32)c2ccccc2n4-c2ccc(-c3nc(-c4ccc5c(c4)oc4ccccc45)nc(-c4cccc5c4sc4ccccc45)n3)cc2)cc1] | 681 |
| 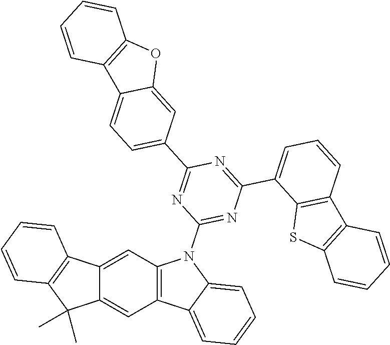 [SMILES: CC1(C)c2ccccc2-c2cc3c(cc21)c1ccccc1n3-c1nc(-c2ccc3c(c2)oc2ccccc23)nc(-c2cccc3c2sc2ccccc23)n1] | 682 |
| 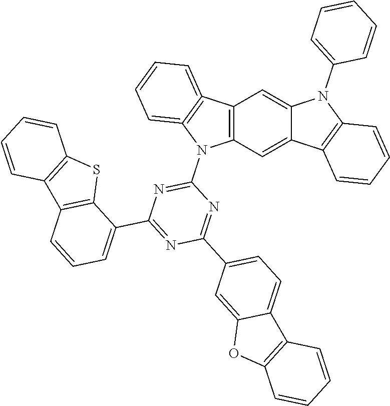 | 683 |
| 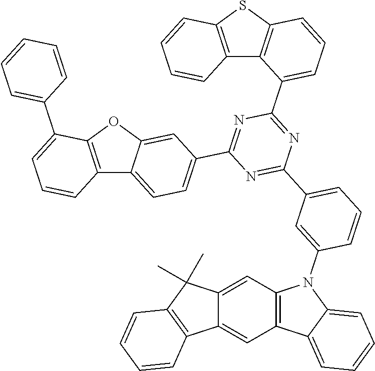 [SMILES: CC1(C)c2ccccc2-c2cc3c4ccccc4n(-c4cccc(-c5nc(-c6ccc7c(c6)oc6c(-c8ccccc8)cccc67)nc(-c6cccc7sc8ccccc8c67)n5)c4)c3cc21] | 684 |
| 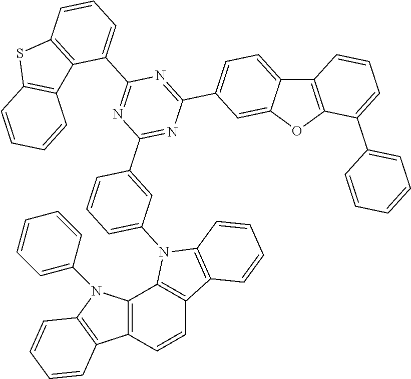 [SMILES: c1ccc(-c2cccc3c2oc2cc(-c4nc(-c5cccc(-n6c7ccccc7c7ccc8c9ccccc9n(-c9ccccc9)c8c76)c5)nc(-c5cccc6sc7ccccc7c56)n4)ccc23)cc1] | 685 |
| 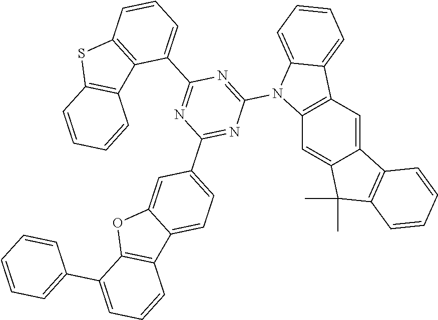 [SMILES: CC1(C)c2ccccc2-c2cc3c4ccccc4n(-c4nc(-c5ccc6c(c5)oc5c(-c7ccccc7)cccc56)nc(-c5cccc6sc7ccccc7c56)n4)c3cc21] | 686 |
| 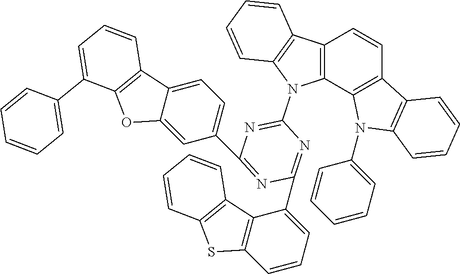 [SMILES: c1ccc(-c2cccc3c2oc2cc(-c4nc(-c5cccc6sc7ccccc7c56)nc(-n5c6ccccc6c6ccc7c8ccccc8n(-c8ccccc8)c7c65)n4)ccc23)cc1] | 687 |
| 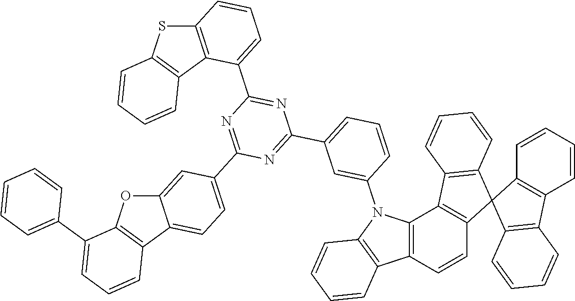 [SMILES: c1ccc(-c2cccc3c2oc2cc(-c4nc(-c5cccc(-n6c7ccccc7c7ccc8c(c76)-c6ccccc6C86c7ccccc7-c7ccccc76)c5)nc(-c5cccc6sc7ccccc7c56)n4)ccc23)cc1] | 688 |
| 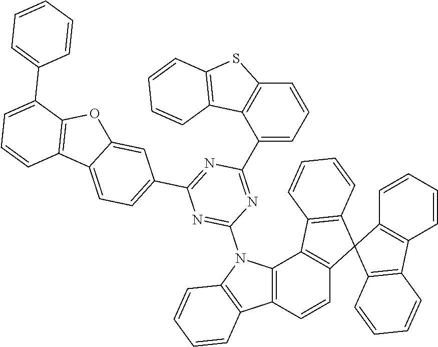 [SMILES: c1ccc(-c2cccc3c2oc2cc(-c4nc(-c5cccc6sc7ccccc7c56)nc(-n5c6ccccc6c6ccc7c(c65)-c5ccccc5C75c6ccccc6-c6ccccc65)n4)ccc23)cc1] | 689 |
| 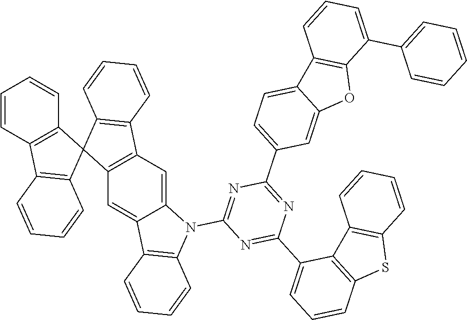 [SMILES: c1ccc(-c2cccc3c2oc2cc(-c4nc(-c5cccc6sc7ccccc7c56)nc(-n5c6ccccc6c6cc7c(cc65)-c5ccccc5C75c6ccccc6-c6ccccc65)n4)ccc23)cc1] | 690 |
| 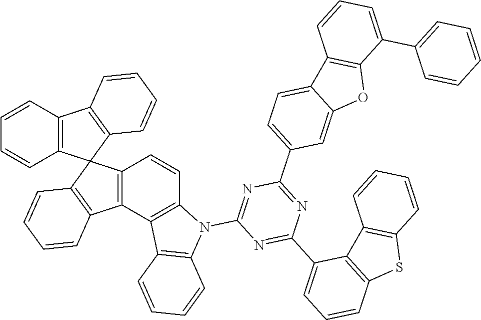 [SMILES: c1ccc(-c2cccc3c2oc2cc(-c4nc(-c5cccc6sc7ccccc7c56)nc(-n5c6ccccc6c6c7c(ccc65)C5(c6ccccc6-c6ccccc65)c5ccccc5-7)n4)ccc23)cc1] | 691 |
| 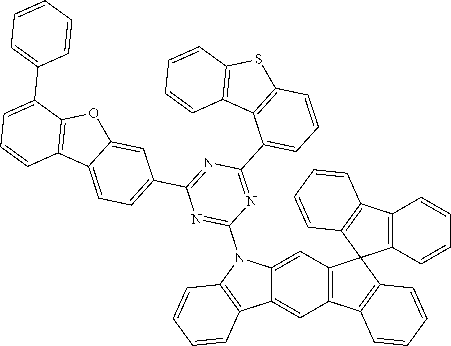 [SMILES: c1ccc(-c2cccc3c2oc2cc(-c4nc(-c5cccc6sc7ccccc7c56)nc(-n5c6ccccc6c6cc7c(cc65)C5(c6ccccc6-c6ccccc65)c5ccccc5-7)n4)ccc23)cc1] | 692 |
| 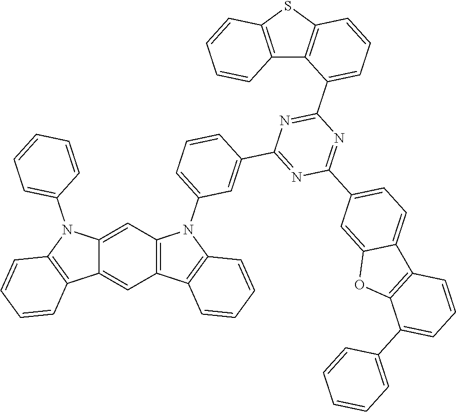 [SMILES: c1ccc(-c2cccc3c2oc2cc(-c4nc(-c5cccc(-n6c7ccccc7c7cc8c9ccccc9n(-c9ccccc9)c8cc76)c5)nc(-c5cccc6sc7ccccc7c56)n4)ccc23)cc1] | 693 |
| 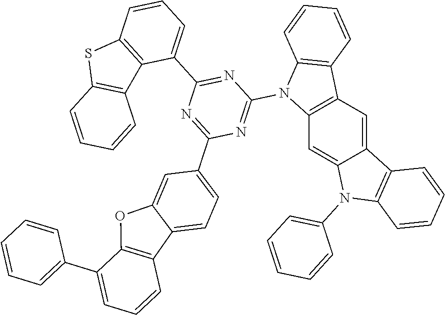 [SMILES: c1ccc(-c2cccc3c2oc2cc(-c4nc(-c5cccc6sc7ccccc7c56)nc(-n5c6ccccc6c6cc7c8ccccc8n(-c8ccccc8)c7cc65)n4)ccc23)cc1] | 694 |
| 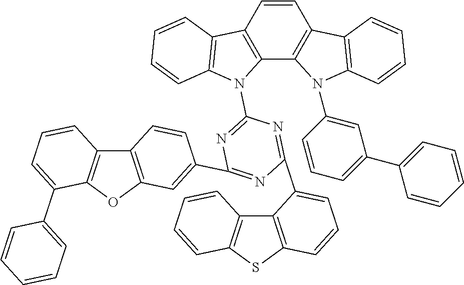 [SMILES: c1ccc(-c2cccc(-n3c4ccccc4c4ccc5c6ccccc6n(-c6nc(-c7ccc8c(c7)oc7c(-c9ccccc9)cccc78)nc(-c7cccc8sc9ccccc9c78)n6)c5c43)c2)cc1] | 695 |
| 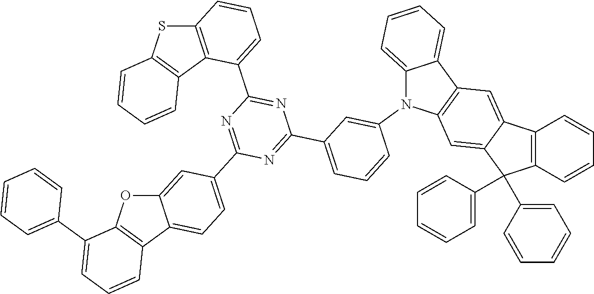 [SMILES: c1ccc(-c2cccc3c2oc2cc(-c4nc(-c5cccc(-n6c7ccccc7c7cc8c(cc76)C(c6ccccc6)(c6ccccc6)c6ccccc6-8)c5)nc(-c5cccc6sc7ccccc7c56)n4)ccc23)cc1] | 696 |
| 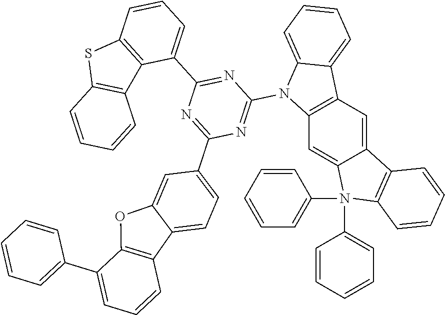 | 697 |
| 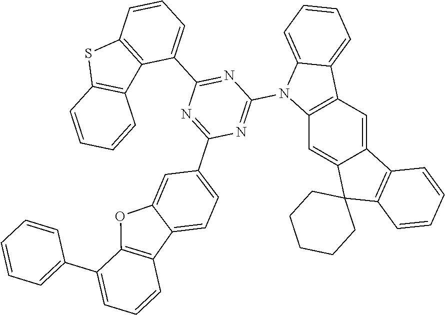 [SMILES: c1ccc(-c2cccc3c2oc2cc(-c4nc(-c5cccc6sc7ccccc7c56)nc(-n5c6ccccc6c6cc7c(cc65)C5(CCCCC5)c5ccccc5-7)n4)ccc23)cc1] | 698 |
| 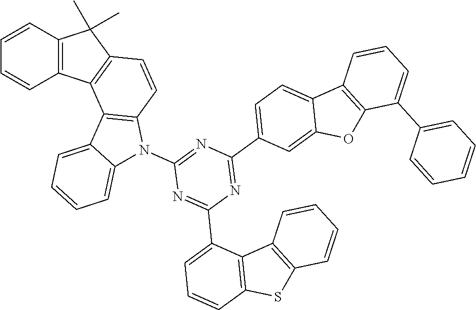 [SMILES: CC1(C)c2ccccc2-c2c1ccc1c2c2ccccc2n1-c1nc(-c2ccc3c(c2)oc2c(-c4ccccc4)cccc23)nc(-c2cccc3sc4ccccc4c23)n1] | 699 |
| 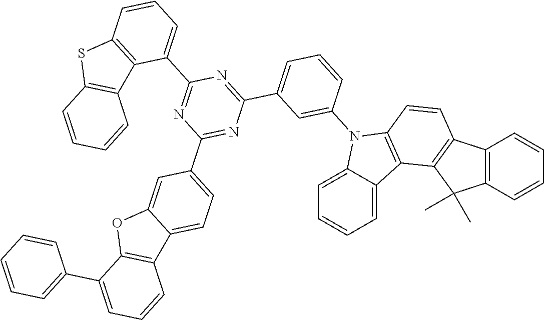 [SMILES: CC1(C)c2ccccc2-c2ccc3c(c21)c1ccccc1n3-c1cccc(-c2nc(-c3ccc4c(c3)oc3c(-c5ccccc5)cccc34)nc(-c3cccc4sc5ccccc5c34)n2)c1] | 700 |
| 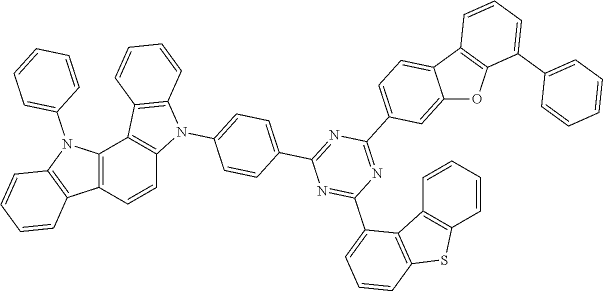 [SMILES: c1ccc(-c2cccc3c2oc2cc(-c4nc(-c5ccc(-n6c7ccccc7c7c6ccc6c8ccccc8n(-c8ccccc8)c67)cc5)nc(-c5cccc6sc7ccccc7c56)n4)ccc23)cc1] | 701 |
| 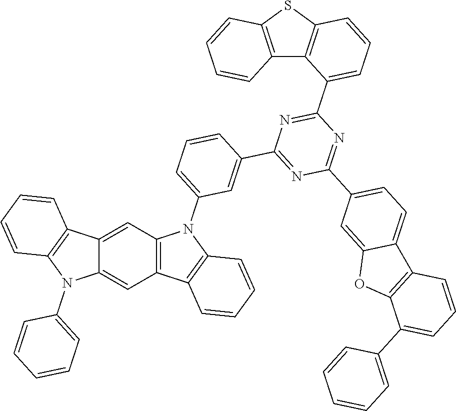 [SMILES: c1ccc(-c2cccc3c2oc2cc(-c4nc(-c5cccc(-n6c7ccccc7c7cc8c(cc76)c6ccccc6n8-c6ccccc6)c5)nc(-c5cccc6sc7ccccc7c56)n4)ccc23)cc1] | 702 |
| 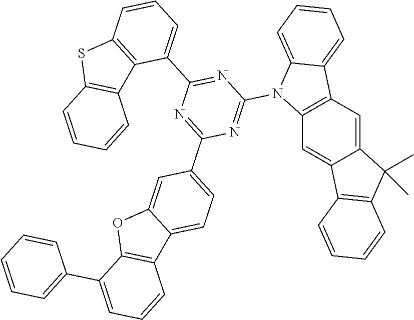 [SMILES: CC1(C)c2ccccc2-c2cc3c(cc21)c1ccccc1n3-c1nc(-c2ccc3c(c2)oc2c(-c4ccccc4)cccc23)nc(-c2cccc3sc4ccccc4c23)n1] | 703 |
| 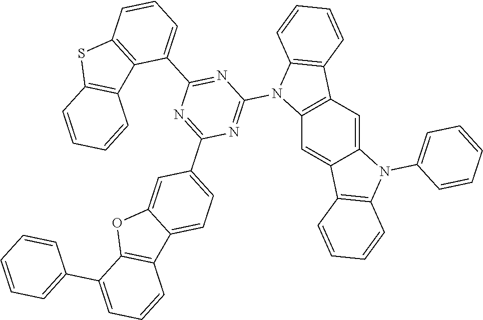 [SMILES: c1ccc(-c2cccc3c2oc2cc(-c4nc(-c5cccc6sc7ccccc7c56)nc(-n5c6ccccc6c6cc7c(cc65)c5ccccc5n7-c5ccccc5)n4)ccc23)cc1] | 704 |
| 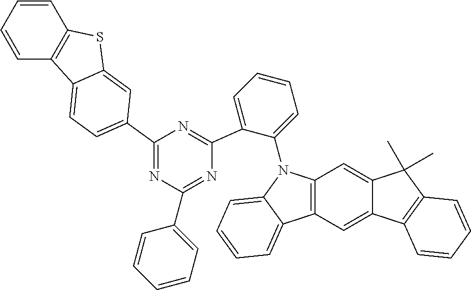 [SMILES: CC1(C)c2ccccc2-c2cc3c4ccccc4n(-c4ccccc4-c4nc(-c5ccccc5)nc(-c5ccc6c(c5)sc5ccccc56)n4)c3cc21] | 705 |
| 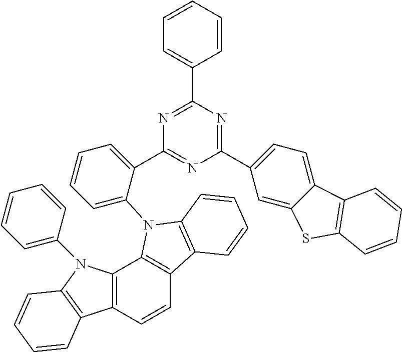 [SMILES: c1ccc(-c2nc(-c3ccc4c(c3)sc3ccccc34)nc(-c3ccccc3-n3c4ccccc4c4ccc5c6ccccc6n(-c6ccccc6)c5c43)n2)cc1] | 706 |
| 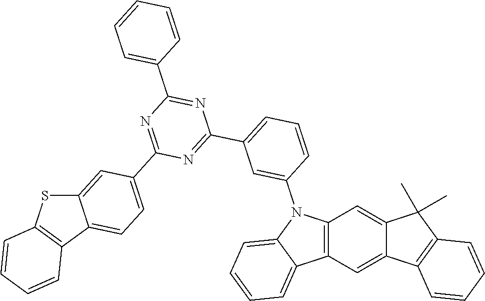 [SMILES: CC1(C)c2ccccc2-c2cc3c4ccccc4n(-c4cccc(-c5nc(-c6ccccc6)nc(-c6ccc7c(c6)sc6ccccc67)n5)c4)c3cc21] | 707 |
| 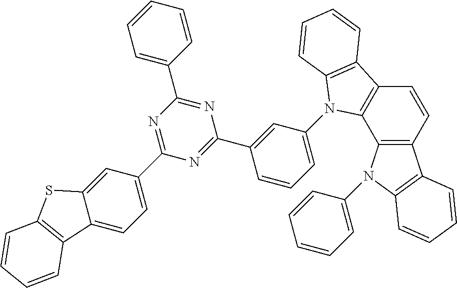 [SMILES: c1ccc(-c2nc(-c3cccc(-n4c5ccccc5c5ccc6c7ccccc7n(-c7ccccc7)c6c54)c3)nc(-c3ccc4c(c3)sc3ccccc34)n2)cc1] | 708 |
| 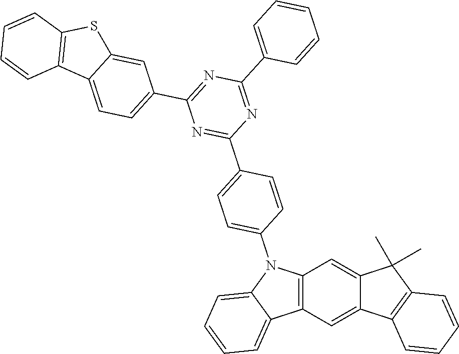 [SMILES: CC1(C)c2ccccc2-c2cc3c4ccccc4n(-c4ccc(-c5nc(-c6ccccc6)nc(-c6ccc7c(c6)sc6ccccc67)n5)cc4)c3cc21] | 709 |
| 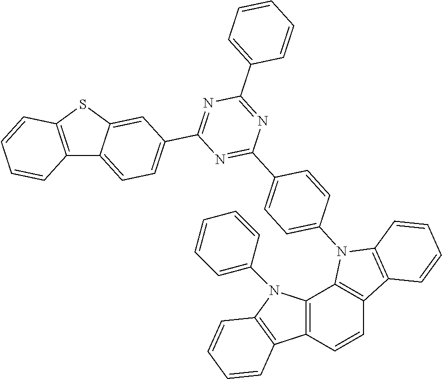 [SMILES: c1ccc(-c2nc(-c3ccc(-n4c5ccccc5c5ccc6c7ccccc7n(-c7ccccc7)c6c54)cc3)nc(-c3ccc4c(c3)sc3ccccc34)n2)cc1] | 710 |
| 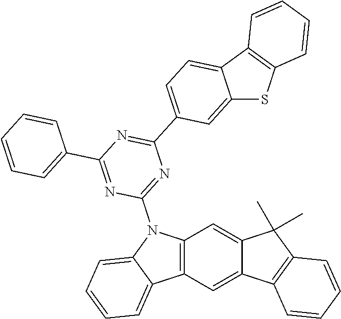 [SMILES: CC1(C)c2ccccc2-c2cc3c4ccccc4n(-c4nc(-c5ccccc5)nc(-c5ccc6c(c5)sc5ccccc56)n4)c3cc21] | 711 |
| 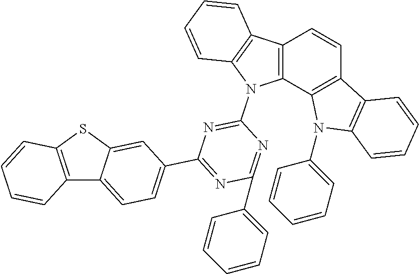 [SMILES: c1ccc(-c2nc(-c3ccc4c(c3)sc3ccccc34)nc(-n3c4ccccc4c4ccc5c6ccccc6n(-c6ccccc6)c5c43)n2)cc1] | 712 |
| 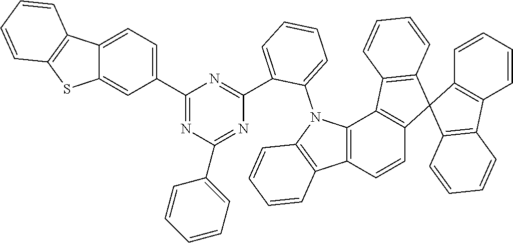 [SMILES: c1ccc(-c2nc(-c3ccc4c(c3)sc3ccccc34)nc(-c3ccccc3-n3c4ccccc4c4ccc5c(c43)-c3ccccc3C53c4ccccc4-c4ccccc43)n2)cc1] | 713 |
| 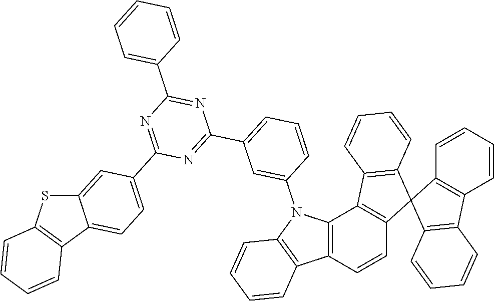 [SMILES: c1ccc(-c2nc(-c3cccc(-n4c5ccccc5c5ccc6c(c54)-c4ccccc4C64c5ccccc5-c5ccccc54)c3)nc(-c3ccc4c(c3)sc3ccccc34)n2)cc1] | 714 |
| 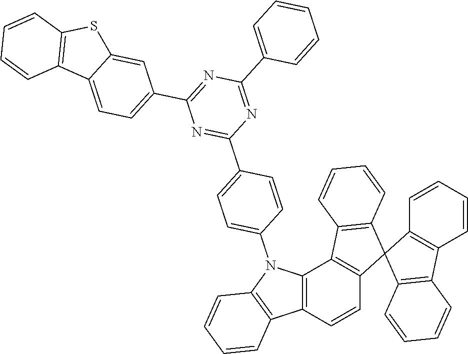 [SMILES: c1ccc(-c2nc(-c3ccc(-n4c5ccccc5c5ccc6c(c54)-c4ccccc4C64c5ccccc5-c5ccccc54)cc3)nc(-c3ccc4c(c3)sc3ccccc34)n2)cc1] | 715 |
| 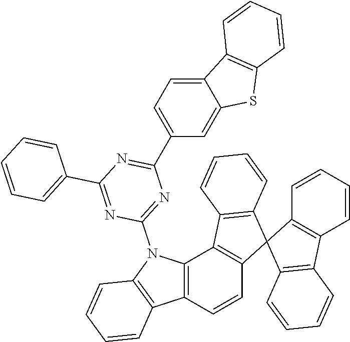 [SMILES: c1ccc(-c2nc(-c3ccc4c(c3)sc3ccccc34)nc(-n3c4ccccc4c4ccc5c(c43)-c3ccccc3C53c4ccccc4-c4ccccc43)n2)cc1] | 716 |
| 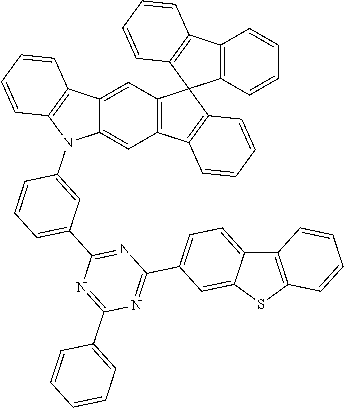 | 717 |
| 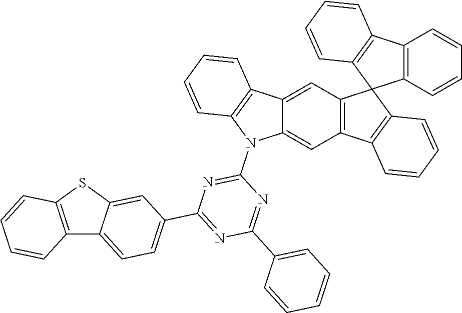 [SMILES: c1ccc(-c2nc(-c3ccc4c(c3)sc3ccccc34)nc(-n3c4ccccc4c4cc5c(cc43)-c3ccccc3C53c4ccccc4-c4ccccc43)n2)cc1] | 718 |
| 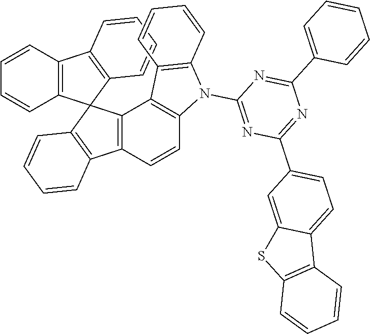 [SMILES: c1ccc(-c2nc(-c3ccc4c(c3)sc3ccccc34)nc(-n3c4ccccc4c4c5c(ccc43)-c3ccccc3C53c4ccccc4-c4ccccc43)n2)cc1] | 719 |
| 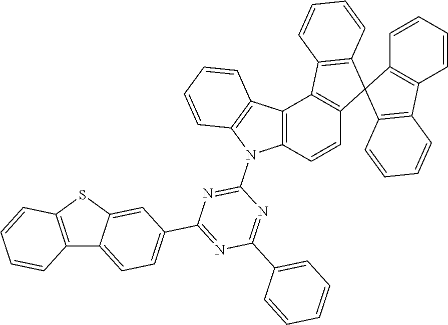 [SMILES: c1ccc(-c2nc(-c3ccc4c(c3)sc3ccccc34)nc(-n3c4ccccc4c4c5c(ccc43)C3(c4ccccc4-c4ccccc43)c3ccccc3-5)n2)cc1] | 720 |
| 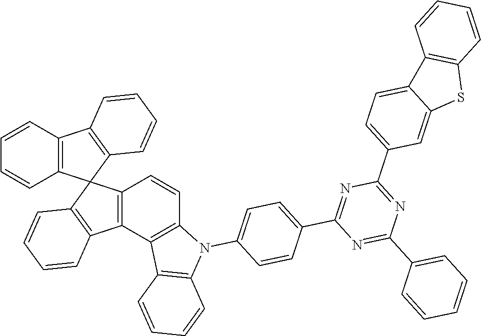 [SMILES: c1ccc(-c2nc(-c3ccc(-n4c5ccccc5c5c6c(ccc54)C4(c5ccccc5-c5ccccc54)c4ccccc4-6)cc3)nc(-c3ccc4c(c3)sc3ccccc34)n2)cc1] | 721 |
| 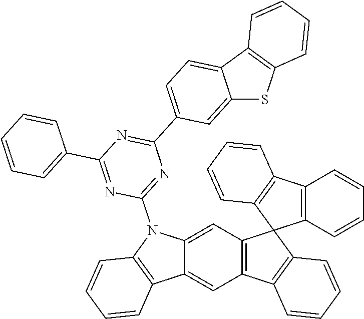 [SMILES: c1ccc(-c2nc(-c3ccc4c(c3)sc3ccccc34)nc(-n3c4ccccc4c4cc5c(cc43)C3(c4ccccc4-c4ccccc43)c3ccccc3-5)n2)cc1] | 722 |
| 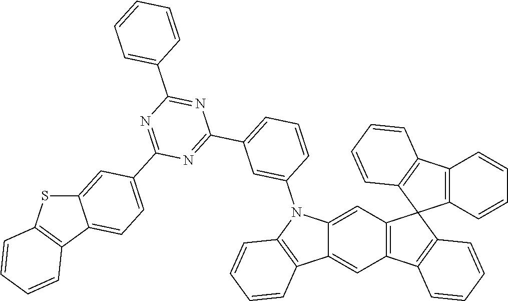 [SMILES: c1ccc(-c2nc(-c3cccc(-n4c5ccccc5c5cc6c(cc54)C4(c5ccccc5-c5ccccc54)c4ccccc4-6)c3)nc(-c3ccc4c(c3)sc3ccccc34)n2)cc1] | 723 |
| 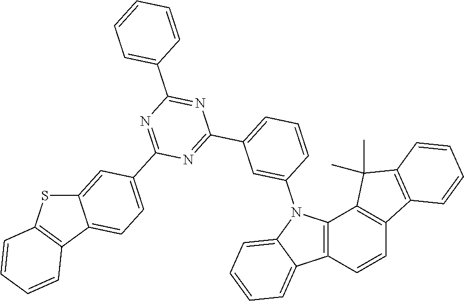 [SMILES: CC1(C)c2ccccc2-c2ccc3c4ccccc4n(-c4cccc(-c5nc(-c6ccccc6)nc(-c6ccc7c(c6)sc6ccccc67)n5)c4)c3c21] | 724 |
| 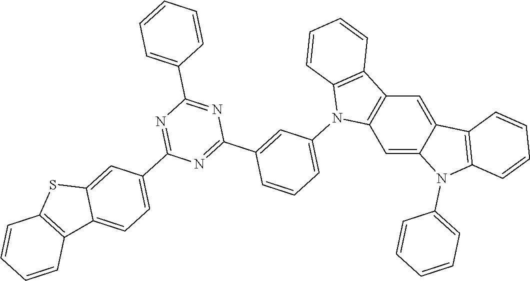 [SMILES: c1ccc(-c2nc(-c3cccc(-n4c5ccccc5c5cc6c7ccccc7n(-c7ccccc7)c6cc54)c3)nc(-c3ccc4c(c3)sc3ccccc34)n2)cc1] | 725 |
| 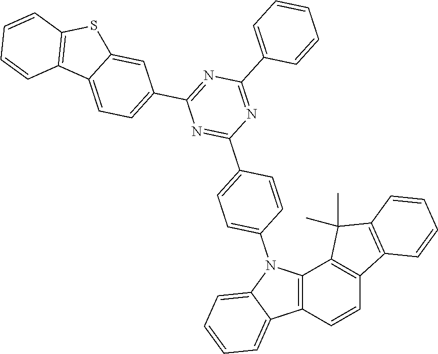 [SMILES: CC1(C)c2ccccc2-c2ccc3c4ccccc4n(-c4ccc(-c5nc(-c6ccccc6)nc(-c6ccc7c(c6)sc6ccccc67)n5)cc4)c3c21] | 726 |
| 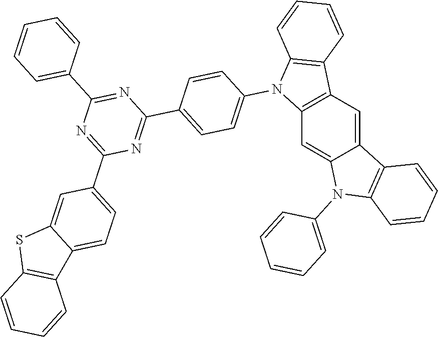 [SMILES: c1ccc(-c2nc(-c3ccc(-n4c5ccccc5c5cc6c7ccccc7n(-c7ccccc7)c6cc54)cc3)nc(-c3ccc4c(c3)sc3ccccc34)n2)cc1] | 727 |
| 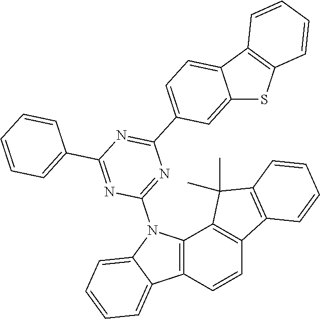 [SMILES: CC1(C)c2ccccc2-c2ccc3c4ccccc4n(-c4nc(-c5ccccc5)nc(-c5ccc6c(c5)sc5ccccc56)n4)c3c21] | 728 |
| 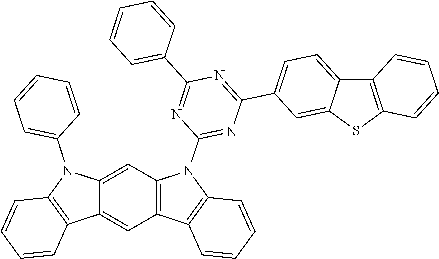 [SMILES: c1ccc(-c2nc(-c3ccc4c(c3)sc3ccccc34)nc(-n3c4ccccc4c4cc5c6ccccc6n(-c6ccccc6)c5cc43)n2)cc1] | 729 |
| 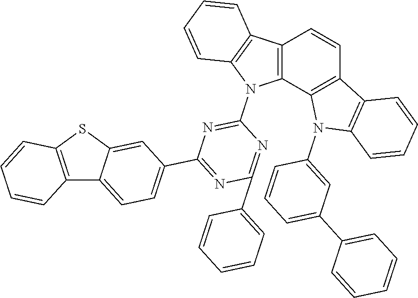 [SMILES: c1ccc(-c2cccc(-n3c4ccccc4c4ccc5c6ccccc6n(-c6nc(-c7ccccc7)nc(-c7ccc8c(c7)sc7ccccc78)n6)c5c43)c2)cc1] | 730 |
| 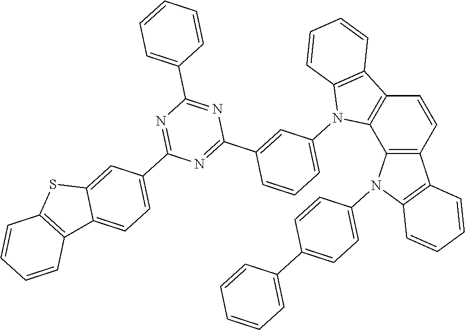 [SMILES: c1ccc(-c2ccc(-n3c4ccccc4c4ccc5c6ccccc6n(-c6cccc(-c7nc(-c8ccccc8)nc(-c8ccc9c(c8)sc8ccccc89)n7)c6)c5c43)cc2)cc1] | 731 |
| 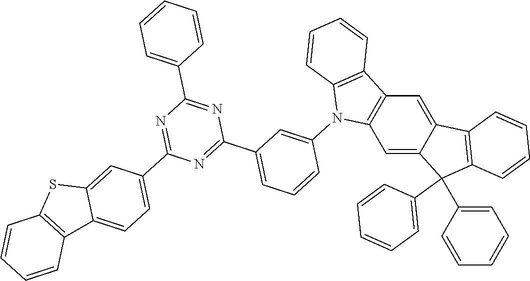 [SMILES: c1ccc(-c2nc(-c3cccc(-n4c5ccccc5c5cc6c(cc54)C(c4ccccc4)(c4ccccc4)c4ccccc4-6)c3)nc(-c3ccc4c(c3)sc3ccccc34)n2)cc1] | 732 |
| 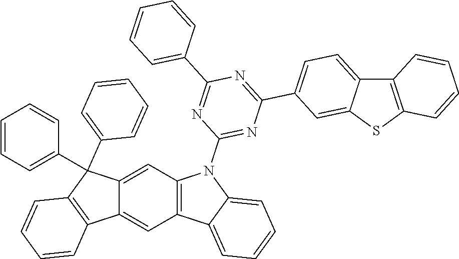 [SMILES: c1ccc(-c2nc(-c3ccc4c(c3)sc3ccccc34)nc(-n3c4ccccc4c4cc5c(cc43)C(c3ccccc3)(c3ccccc3)c3ccccc3-5)n2)cc1] | 733 |
| 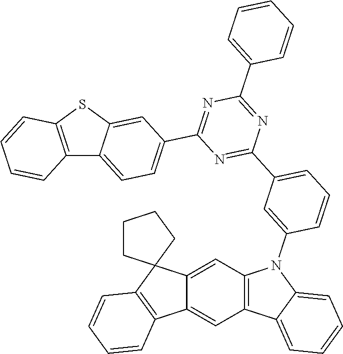 [SMILES: c1ccc(-c2nc(-c3cccc(-n4c5ccccc5c5cc6c(cc54)C4(CCCC4)c4ccccc4-6)c3)nc(-c3ccc4c(c3)sc3ccccc34)n2)cc1] | 734 |
| 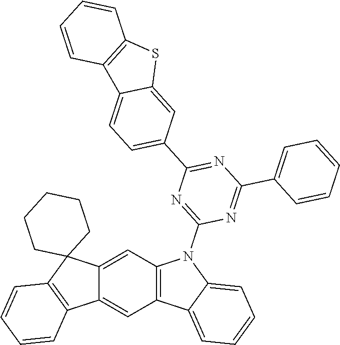 [SMILES: c1ccc(-c2nc(-c3ccc4c(c3)sc3ccccc34)nc(-n3c4ccccc4c4cc5c(cc43)C3(CCCCC3)c3ccccc3-5)n2)cc1] | 735 |
| 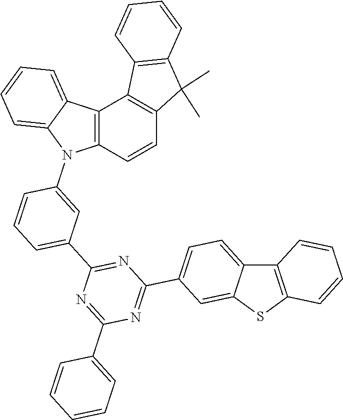 [SMILES: CC1(C)c2ccccc2-c2c1ccc1c2c2ccccc2n1-c1cccc(-c2nc(-c3ccccc3)nc(-c3ccc4c(c3)sc3ccccc34)n2)c1] | 736 |
| 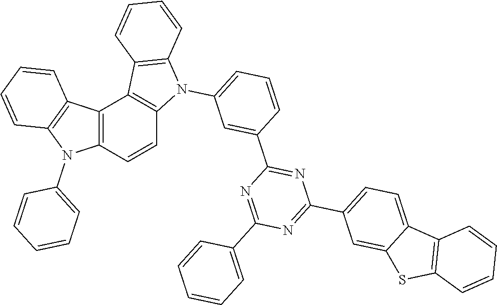 [SMILES: c1ccc(-c2nc(-c3cccc(-n4c5ccccc5c5c6c7ccccc7n(-c7ccccc7)c6ccc54)c3)nc(-c3ccc4c(c3)sc3ccccc34)n2)cc1] | 737 |
| 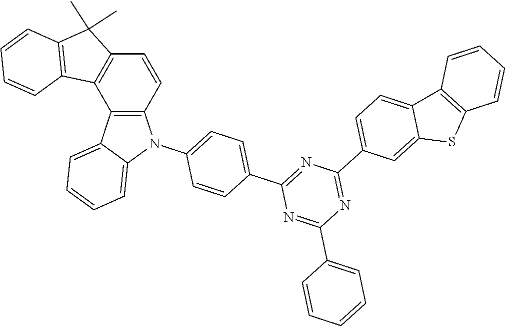 [SMILES: CC1(C)c2ccccc2-c2c1ccc1c2c2ccccc2n1-c1ccc(-c2nc(-c3ccccc3)nc(-c3ccc4c(c3)sc3ccccc34)n2)cc1] | 738 |
| 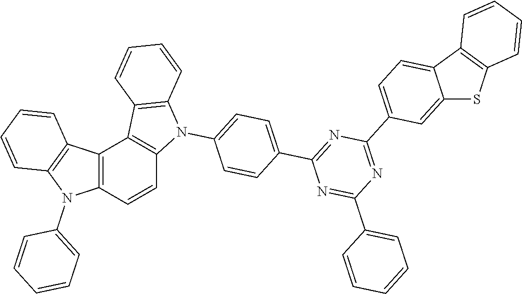 [SMILES: c1ccc(-c2nc(-c3ccc(-n4c5ccccc5c5c6c7ccccc7n(-c7ccccc7)c6ccc54)cc3)nc(-c3ccc4c(c3)sc3ccccc34)n2)cc1] | 739 |
| 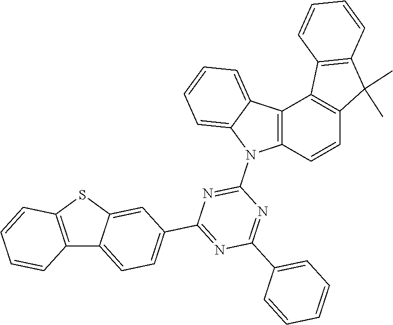 [SMILES: CC1(C)c2ccccc2-c2c1ccc1c2c2ccccc2n1-c1nc(-c2ccccc2)nc(-c2ccc3c(c2)sc2ccccc23)n1] | 740 |
| 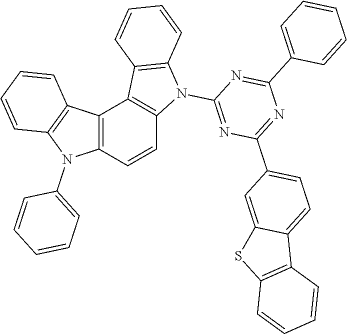 [SMILES: c1ccc(-c2nc(-c3ccc4c(c3)sc3ccccc34)nc(-n3c4ccccc4c4c5c6ccccc6n(-c6ccccc6)c5ccc43)n2)cc1] | 741 |
| 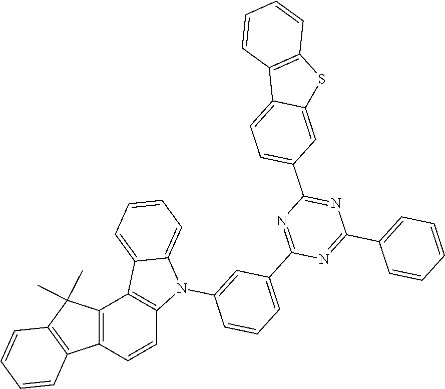 [SMILES: CC1(C)c2ccccc2-c2ccc3c(c21)c1ccccc1n3-c1cccc(-c2nc(-c3ccccc3)nc(-c3ccc4c(c3)sc3ccccc34)n2)c1] | 742 |
| 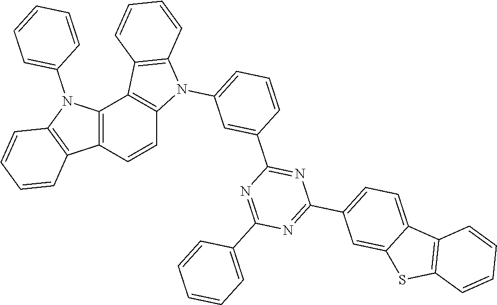 [SMILES: c1ccc(-c2nc(-c3cccc(-n4c5ccccc5c5c4ccc4c6ccccc6n(-c6ccccc6)c45)c3)nc(-c3ccc4c(c3)sc3ccccc34)n2)cc1] | 743 |
| 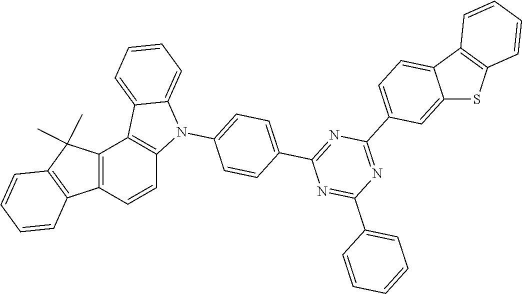 [SMILES: CC1(C)c2ccccc2-c2ccc3c(c21)c1ccccc1n3-c1ccc(-c2nc(-c3ccccc3)nc(-c3ccc4c(c3)sc3ccccc34)n2)cc1] | 744 |
| 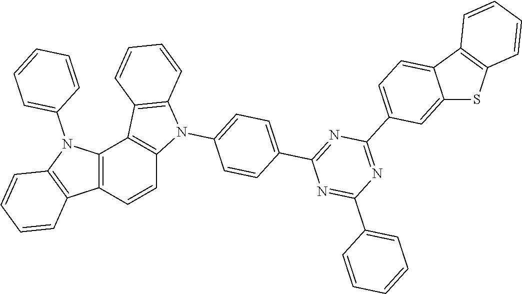 [SMILES: c1ccc(-c2nc(-c3ccc(-n4c5ccccc5c5c4ccc4c6ccccc6n(-c6ccccc6)c45)cc3)nc(-c3ccc4c(c3)sc3ccccc34)n2)cc1] | 745 |
| 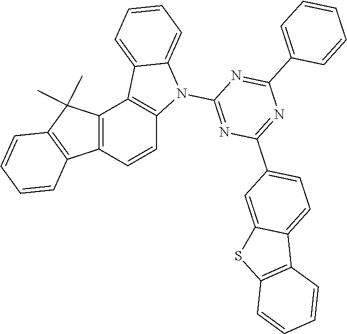 [SMILES: CC1(C)c2ccccc2-c2ccc3c(c21)c1ccccc1n3-c1nc(-c2ccccc2)nc(-c2ccc3c(c2)sc2ccccc23)n1] | 746 |
| 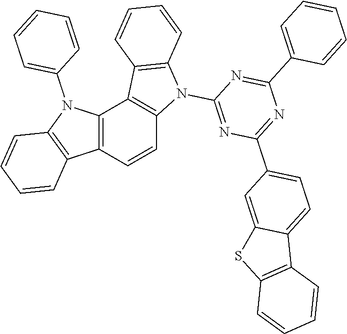 [SMILES: c1ccc(-c2nc(-c3ccc4c(c3)sc3ccccc34)nc(-n3c4ccccc4c4c3ccc3c5ccccc5n(-c5ccccc5)c34)n2)cc1] | 747 |
| 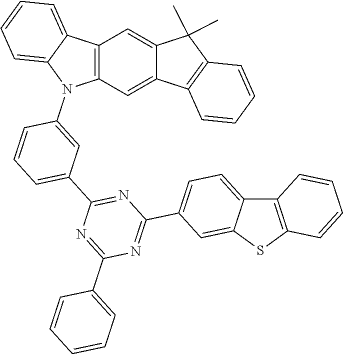 [SMILES: CC1(C)c2ccccc2-c2cc3c(cc21)c1ccccc1n3-c1cccc(-c2nc(-c3ccccc3)nc(-c3ccc4c(c3)sc3ccccc34)n2)c1] | 748 |
| 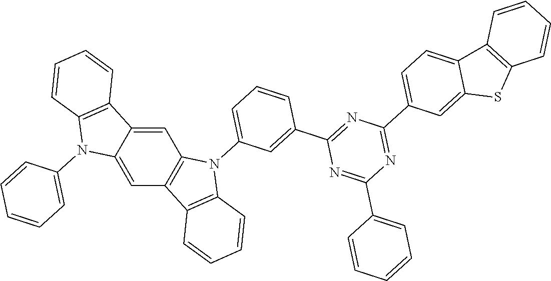 [SMILES: c1ccc(-c2nc(-c3cccc(-n4c5ccccc5c5cc6c(cc54)c4ccccc4n6-c4ccccc4)c3)nc(-c3ccc4c(c3)sc3ccccc34)n2)cc1] | 749 |
| 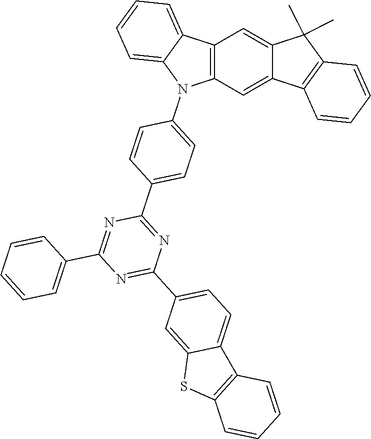 [SMILES: CC1(C)c2ccccc2-c2cc3c(cc21)c1ccccc1n3-c1ccc(-c2nc(-c3ccccc3)nc(-c3ccc4c(c3)sc3ccccc34)n2)cc1] | 750 |
| 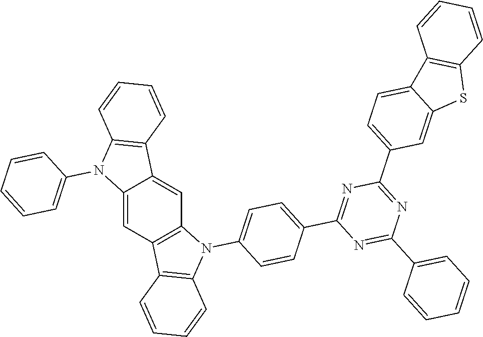 [SMILES: c1ccc(-c2nc(-c3ccc(-n4c5ccccc5c5cc6c(cc54)c4ccccc4n6-c4ccccc4)cc3)nc(-c3ccc4c(c3)sc3ccccc34)n2)cc1] | 751 |
| 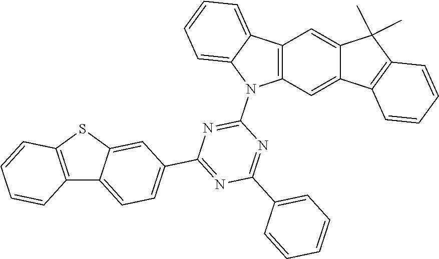 [SMILES: CC1(C)c2ccccc2-c2cc3c(cc21)c1ccccc1n3-c1nc(-c2ccccc2)nc(-c2ccc3c(c2)sc2ccccc23)n1] | 752 |
| 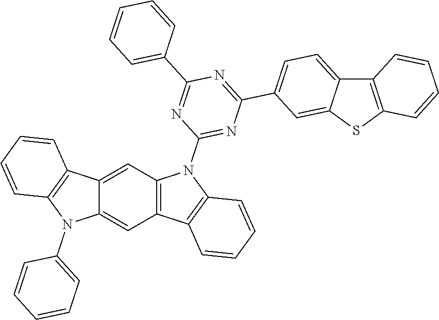 [SMILES: c1ccc(-c2nc(-c3ccc4c(c3)sc3ccccc34)nc(-n3c4ccccc4c4cc5c(cc43)c3ccccc3n5-c3ccccc3)n2)cc1] | 753 |
| 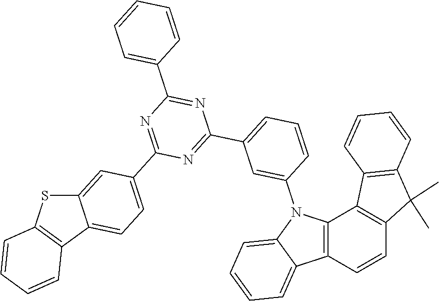 [SMILES: CC1(C)c2ccccc2-c2c1ccc1c3ccccc3n(-c3cccc(-c4nc(-c5ccccc5)nc(-c5ccc6c(c5)sc5ccccc56)n4)c3)c21] | 754 |
| 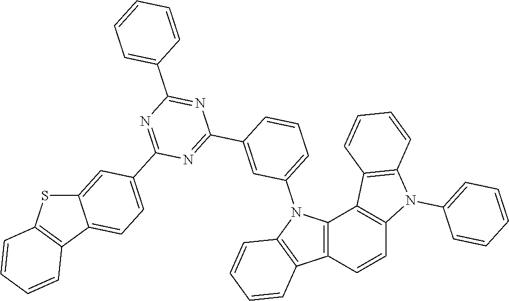 [SMILES: c1ccc(-c2nc(-c3cccc(-n4c5ccccc5c5ccc6c(c7ccccc7n6-c6ccccc6)c54)c3)nc(-c3ccc4c(c3)sc3ccccc34)n2)cc1] | 755 |
| 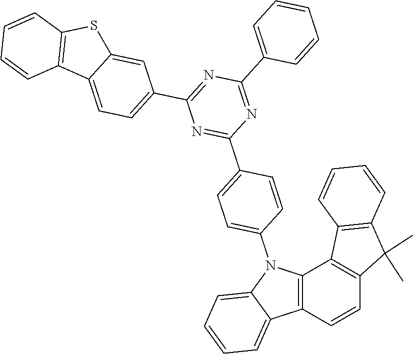 [SMILES: CC1(C)c2ccccc2-c2c1ccc1c3ccccc3n(-c3ccc(-c4nc(-c5ccccc5)nc(-c5ccc6c(c5)sc5ccccc56)n4)cc3)c21] | 756 |
| 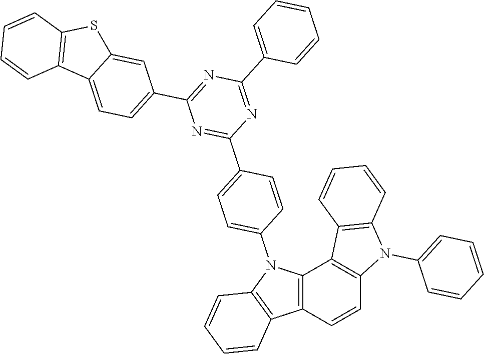 [SMILES: c1ccc(-c2nc(-c3ccc(-n4c5ccccc5c5ccc6c(c7ccccc7n6-c6ccccc6)c54)cc3)nc(-c3ccc4c(c3)sc3ccccc34)n2)cc1] | 757 |
| 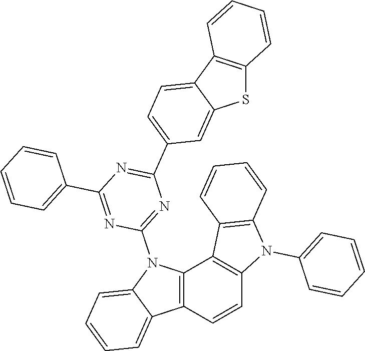 [SMILES: c1ccc(-c2nc(-c3ccc4c(c3)sc3ccccc34)nc(-n3c4ccccc4c4ccc5c(c6ccccc6n5-c5ccccc5)c43)n2)cc1] | 758 |
| 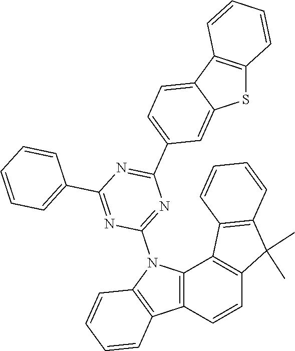 [SMILES: CC1(C)c2ccccc2-c2c1ccc1c3ccccc3n(-c3nc(-c4ccccc4)nc(-c4ccc5c(c4)sc4ccccc45)n3)c21] | 759 |
| 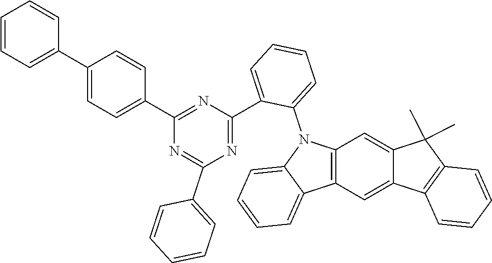 [SMILES: CC1(C)c2ccccc2-c2cc3c4ccccc4n(-c4ccccc4-c4nc(-c5ccccc5)nc(-c5ccc(-c6ccccc6)cc5)n4)c3cc21] | 760 |
| 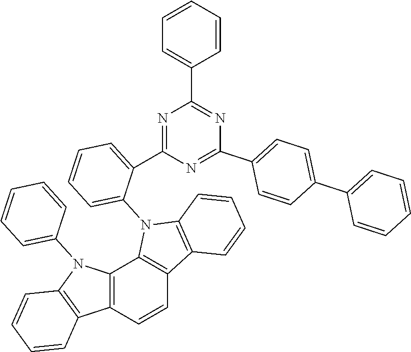 [SMILES: c1ccc(-c2ccc(-c3nc(-c4ccccc4)nc(-c4ccccc4-n4c5ccccc5c5ccc6c7ccccc7n(-c7ccccc7)c6c54)n3)cc2)cc1] | 761 |
| 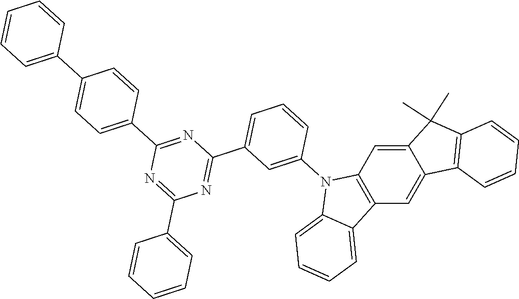 [SMILES: CC1(C)c2ccccc2-c2cc3c4ccccc4n(-c4cccc(-c5nc(-c6ccccc6)nc(-c6ccc(-c7ccccc7)cc6)n5)c4)c3cc21] | 762 |
| 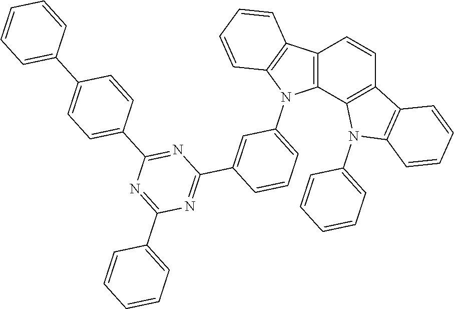 [SMILES: c1ccc(-c2ccc(-c3nc(-c4ccccc4)nc(-c4cccc(-n5c6ccccc6c6ccc7c8ccccc8n(-c8ccccc8)c7c65)c4)n3)cc2)cc1] | 763 |
| 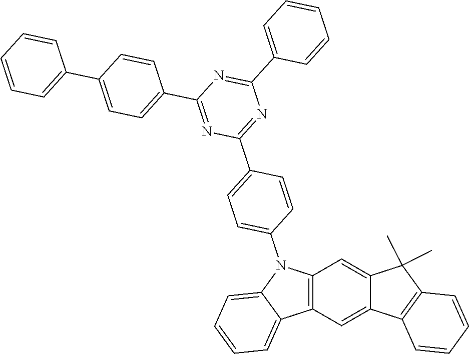 [SMILES: CC1(C)c2ccccc2-c2cc3c4ccccc4n(-c4ccc(-c5nc(-c6ccccc6)nc(-c6ccc(-c7ccccc7)cc6)n5)cc4)c3cc21] | 764 |
| 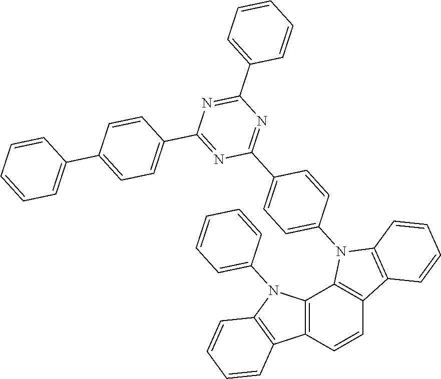 [SMILES: c1ccc(-c2ccc(-c3nc(-c4ccccc4)nc(-c4ccc(-n5c6ccccc6c6ccc7c8ccccc8n(-c8ccccc8)c7c65)cc4)n3)cc2)cc1] | 765 |
| 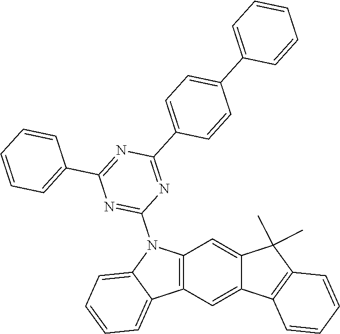 [SMILES: CC1(C)c2ccccc2-c2cc3c4ccccc4n(-c4nc(-c5ccccc5)nc(-c5ccc(-c6ccccc6)cc5)n4)c3cc21] | 766 |
| 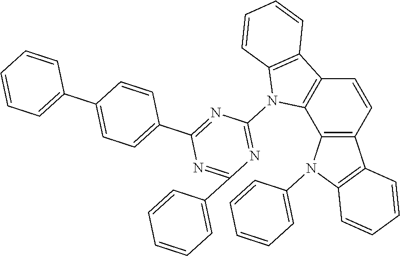 [SMILES: c1ccc(-c2ccc(-c3nc(-c4ccccc4)nc(-n4c5ccccc5c5ccc6c7ccccc7n(-c7ccccc7)c6c54)n3)cc2)cc1] | 767 |
| 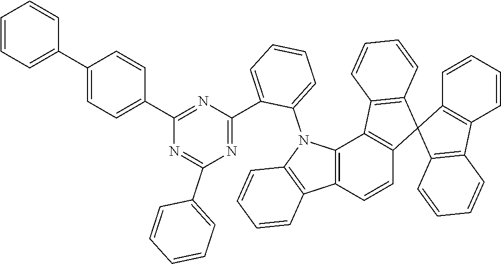 [SMILES: c1ccc(-c2ccc(-c3nc(-c4ccccc4)nc(-c4ccccc4-n4c5ccccc5c5ccc6c(c54)-c4ccccc4C64c5ccccc5-c5ccccc54)n3)cc2)cc1] | 768 |
| 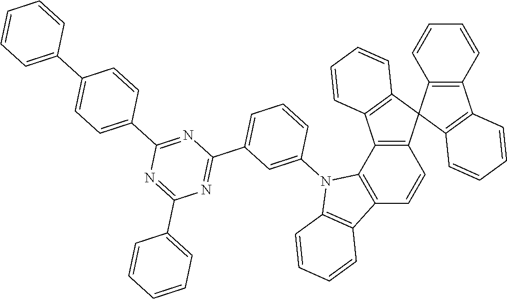 [SMILES: c1ccc(-c2ccc(-c3nc(-c4ccccc4)nc(-c4cccc(-n5c6ccccc6c6ccc7c(c65)-c5ccccc5C75c6ccccc6-c6ccccc65)c4)n3)cc2)cc1] | 769 |
| 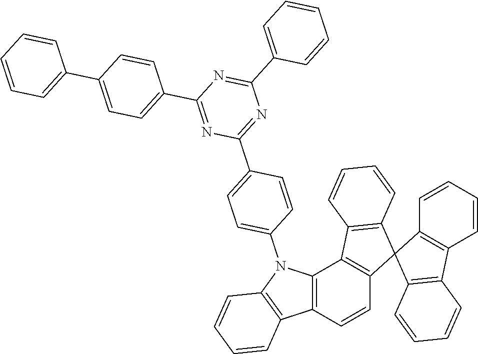 [SMILES: c1ccc(-c2ccc(-c3nc(-c4ccccc4)nc(-c4ccc(-n5c6ccccc6c6ccc7c(c65)-c5ccccc5C75c6ccccc6-c6ccccc65)cc4)n3)cc2)cc1] | 770 |
| 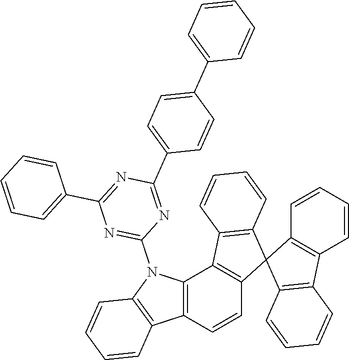 [SMILES: c1ccc(-c2ccc(-c3nc(-c4ccccc4)nc(-n4c5ccccc5c5ccc6c(c54)-c4ccccc4C64c5ccccc5-c5ccccc54)n3)cc2)cc1] | 771 |
| 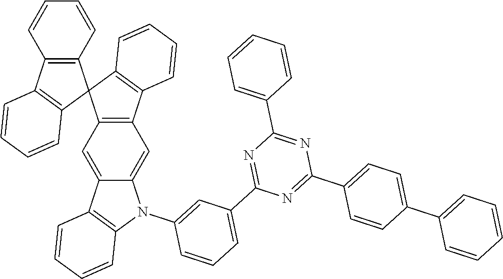 [SMILES: c1ccc(-c2ccc(-c3nc(-c4ccccc4)nc(-c4cccc(-n5c6ccccc6c6cc7c(cc65)-c5ccccc5C75c6ccccc6-c6ccccc65)c4)n3)cc2)cc1] | 772 |
| 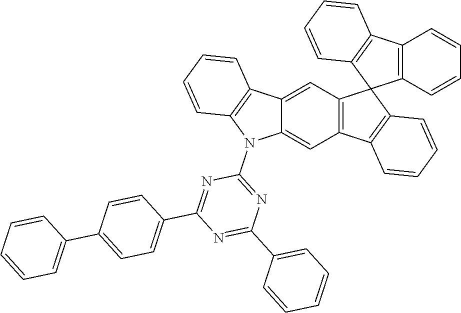 [SMILES: c1ccc(-c2ccc(-c3nc(-c4ccccc4)nc(-n4c5ccccc5c5cc6c(cc54)-c4ccccc4C64c5ccccc5-c5ccccc54)n3)cc2)cc1] | 773 |
| 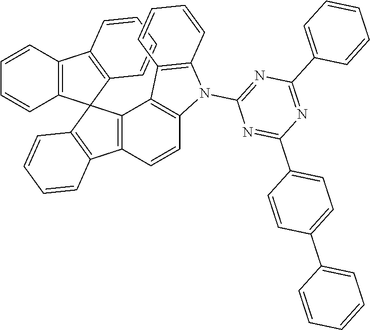 [SMILES: c1ccc(-c2ccc(-c3nc(-c4ccccc4)nc(-n4c5ccccc5c5c6c(ccc54)-c4ccccc4C64c5ccccc5-c5ccccc54)n3)cc2)cc1] | 774 |
| 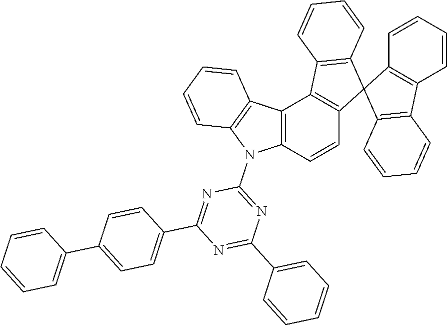 [SMILES: c1ccc(-c2ccc(-c3nc(-c4ccccc4)nc(-n4c5ccccc5c5c6c(ccc54)C4(c5ccccc5-c5ccccc54)c4ccccc4-6)n3)cc2)cc1] | 775 |
| 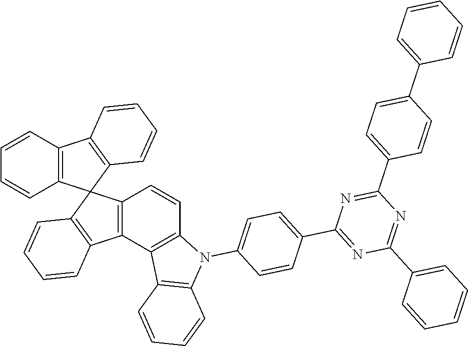 [SMILES: c1ccc(-c2ccc(-c3nc(-c4ccccc4)nc(-c4ccc(-n5c6ccccc6c6c7c(ccc65)C5(c6ccccc6-c6ccccc65)c5ccccc5-7)cc4)n3)cc2)cc1] | 776 |
| 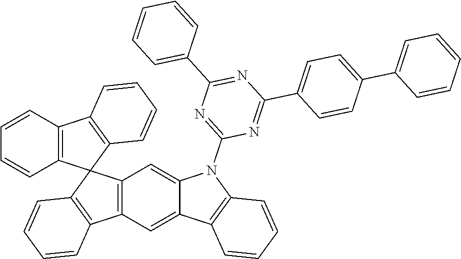 [SMILES: c1ccc(-c2ccc(-c3nc(-c4ccccc4)nc(-n4c5ccccc5c5cc6c(cc54)C4(c5ccccc5-c5ccccc54)c4ccccc4-6)n3)cc2)cc1] | 777 |
| 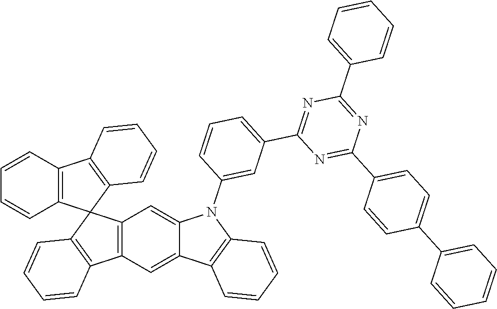 [SMILES: c1ccc(-c2ccc(-c3nc(-c4ccccc4)nc(-c4cccc(-n5c6ccccc6c6cc7c(cc65)C5(c6ccccc6-c6ccccc65)c5ccccc5-7)c4)n3)cc2)cc1] | 778 |
| 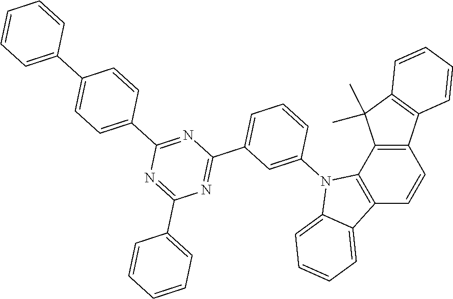 [SMILES: CC1(C)c2ccccc2-c2ccc3c4ccccc4n(-c4cccc(-c5nc(-c6ccccc6)nc(-c6ccc(-c7ccccc7)cc6)n5)c4)c3c21] | 779 |
| 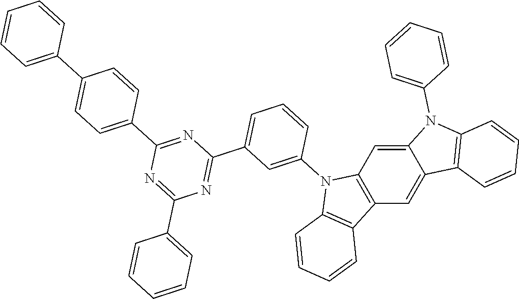 [SMILES: c1ccc(-c2ccc(-c3nc(-c4ccccc4)nc(-c4cccc(-n5c6ccccc6c6cc7c8ccccc8n(-c8ccccc8)c7cc65)c4)n3)cc2)cc1] | 780 |
| 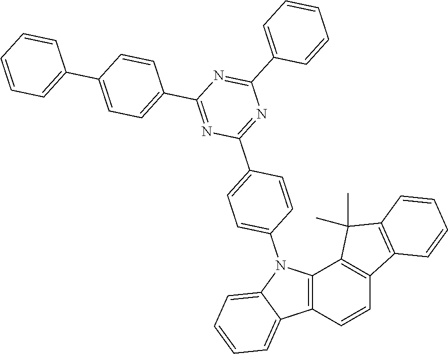 [SMILES: CC1(C)c2ccccc2-c2ccc3c4ccccc4n(-c4ccc(-c5nc(-c6ccccc6)nc(-c6ccc(-c7ccccc7)cc6)n5)cc4)c3c21] | 781 |
| 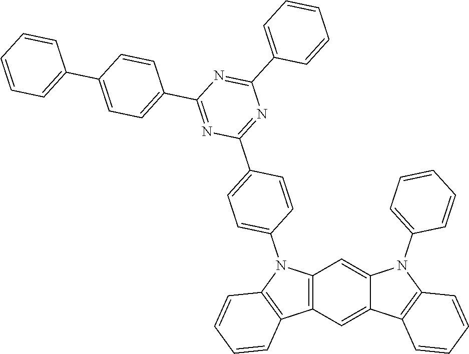 [SMILES: c1ccc(-c2ccc(-c3nc(-c4ccccc4)nc(-c4ccc(-n5c6ccccc6c6cc7c8ccccc8n(-c8ccccc8)c7cc65)cc4)n3)cc2)cc1] | 782 |
| 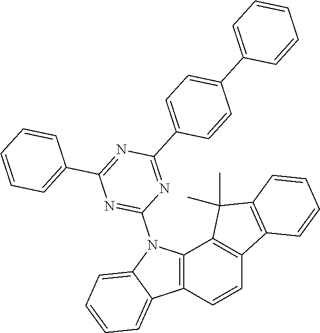 [SMILES: CC1(C)c2ccccc2-c2ccc3c4ccccc4n(-c4nc(-c5ccccc5)nc(-c5ccc(-c6ccccc6)cc5)n4)c3c21] | 783 |
| 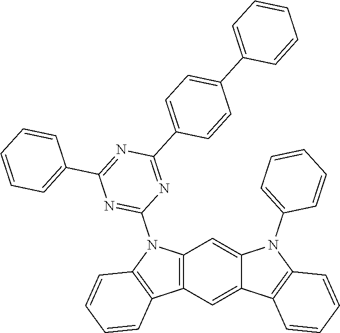 [SMILES: c1ccc(-c2ccc(-c3nc(-c4ccccc4)nc(-n4c5ccccc5c5cc6c7ccccc7n(-c7ccccc7)c6cc54)n3)cc2)cc1] | 784 |
| 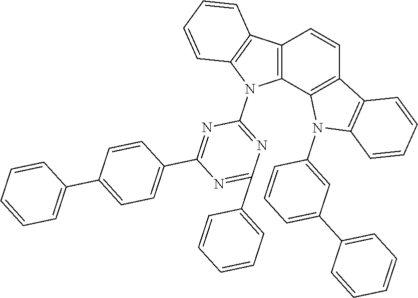 [SMILES: c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1] | 785 |
| 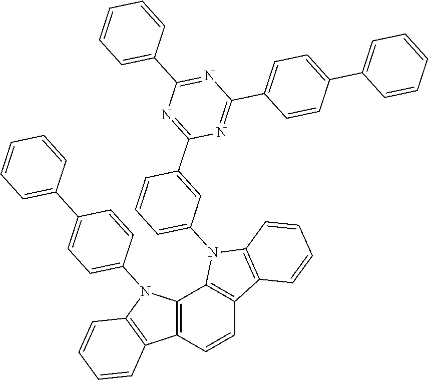 [SMILES: c1ccc(-c2ccc(-c3nc(-c4ccccc4)nc(-c4cccc(-n5c6ccccc6c6ccc7c8ccccc8n(-c8ccc(-c9ccccc9)cc8)c7c65)c4)n3)cc2)cc1] | 786 |
| 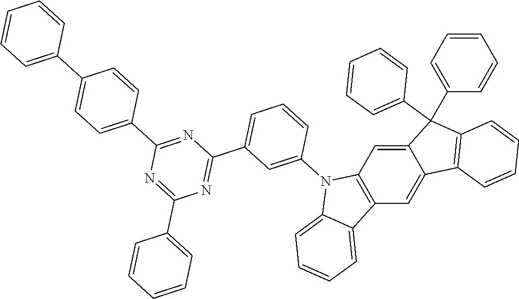 [SMILES: c1ccc(-c2ccc(-c3nc(-c4ccccc4)nc(-c4cccc(-n5c6ccccc6c6cc7c(cc65)C(c5ccccc5)(c5ccccc5)c5ccccc5-7)c4)n3)cc2)cc1] | 787 |
| 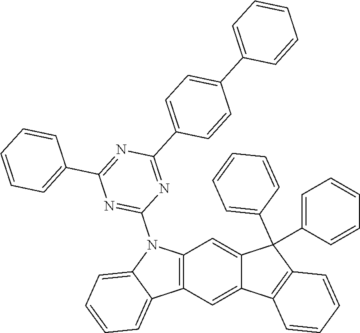 [SMILES: c1ccc(-c2ccc(-c3nc(-c4ccccc4)nc(-n4c5ccccc5c5cc6c(cc54)C(c4ccccc4)(c4ccccc4)c4ccccc4-6)n3)cc2)cc1] | 788 |
| 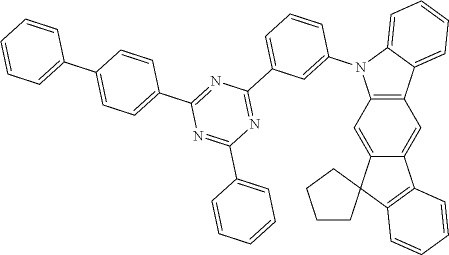 [SMILES: c1ccc(-c2ccc(-c3nc(-c4ccccc4)nc(-c4cccc(-n5c6ccccc6c6cc7c(cc65)C5(CCCC5)c5ccccc5-7)c4)n3)cc2)cc1] | 789 |
| 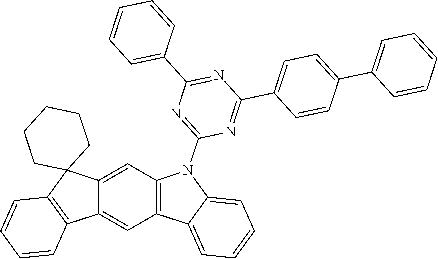 [SMILES: c1ccc(-c2ccc(-c3nc(-c4ccccc4)nc(-n4c5ccccc5c5cc6c(cc54)C4(CCCCC4)c4ccccc4-6)n3)cc2)cc1] | 790 |
| 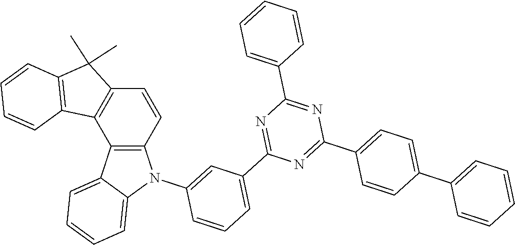 [SMILES: CC1(C)c2ccccc2-c2c1ccc1c2c2ccccc2n1-c1cccc(-c2nc(-c3ccccc3)nc(-c3ccc(-c4ccccc4)cc3)n2)c1] | 791 |
| 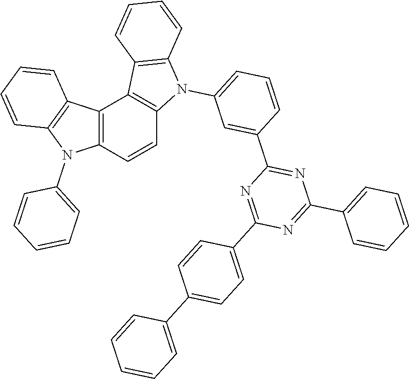 [SMILES: c1ccc(-c2ccc(-c3nc(-c4ccccc4)nc(-c4cccc(-n5c6ccccc6c6c7c8ccccc8n(-c8ccccc8)c7ccc65)c4)n3)cc2)cc1] | 792 |
| 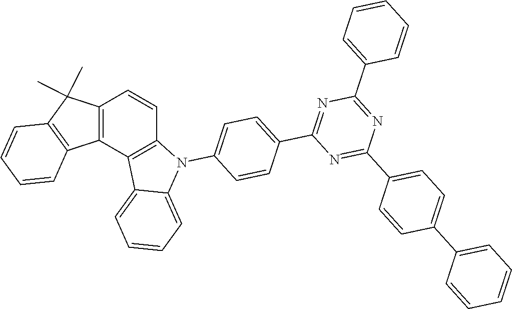 [SMILES: CC1(C)c2ccccc2-c2c1ccc1c2c2ccccc2n1-c1ccc(-c2nc(-c3ccccc3)nc(-c3ccc(-c4ccccc4)cc3)n2)cc1] | 793 |
| 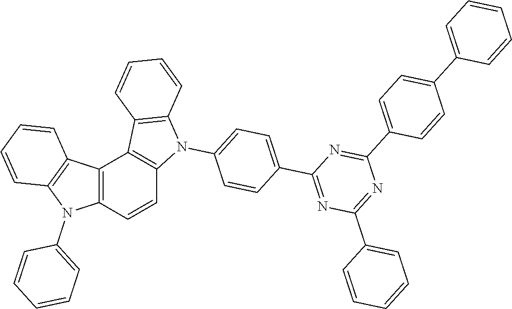 [SMILES: c1ccc(-c2ccc(-c3nc(-c4ccccc4)nc(-c4ccc(-n5c6ccccc6c6c7c8ccccc8n(-c8ccccc8)c7ccc65)cc4)n3)cc2)cc1] | 794 |
| 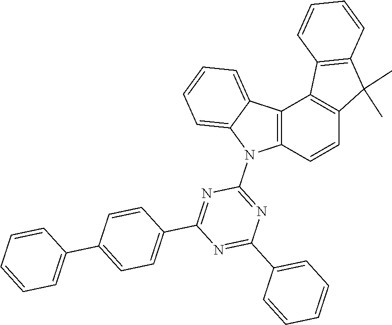 [SMILES: CC1(C)c2ccccc2-c2c1ccc1c2c2ccccc2n1-c1nc(-c2ccccc2)nc(-c2ccc(-c3ccccc3)cc2)n1] | 795 |
| 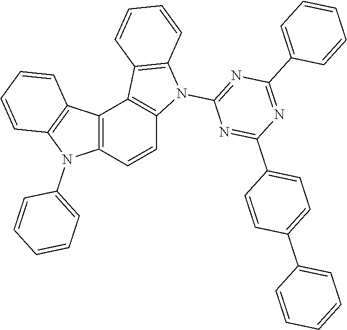 [SMILES: c1ccc(-c2ccc(-c3nc(-c4ccccc4)nc(-n4c5ccccc5c5c6c7ccccc7n(-c7ccccc7)c6ccc54)n3)cc2)cc1] | 796 |
| 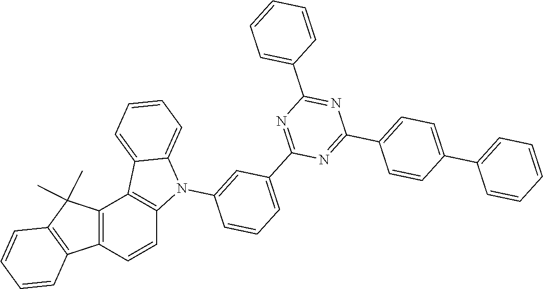 [SMILES: CC1(C)c2ccccc2-c2ccc3c(c21)c1ccccc1n3-c1cccc(-c2nc(-c3ccccc3)nc(-c3ccc(-c4ccccc4)cc3)n2)c1] | 797 |
| 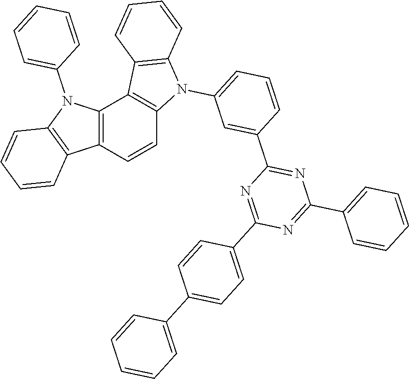 [SMILES: c1ccc(-c2ccc(-c3nc(-c4ccccc4)nc(-c4cccc(-n5c6ccccc6c6c5ccc5c7ccccc7n(-c7ccccc7)c56)c4)n3)cc2)cc1] | 798 |
| 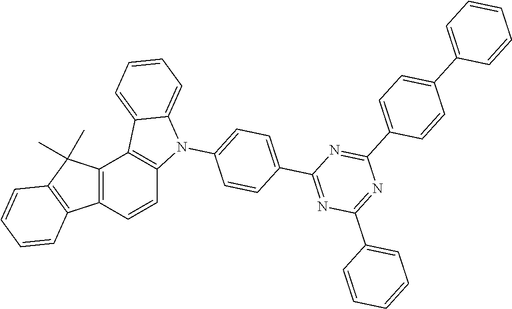 [SMILES: CC1(C)c2ccccc2-c2ccc3c(c21)c1ccccc1n3-c1ccc(-c2nc(-c3ccccc3)nc(-c3ccc(-c4ccccc4)cc3)n2)cc1] | 799 |
| 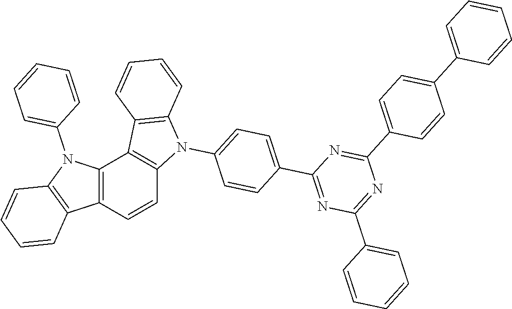 [SMILES: c1ccc(-c2ccc(-c3nc(-c4ccccc4)nc(-c4ccc(-n5c6ccccc6c6c5ccc5c7ccccc7n(-c7ccccc7)c56)cc4)n3)cc2)cc1] | 800 |
| 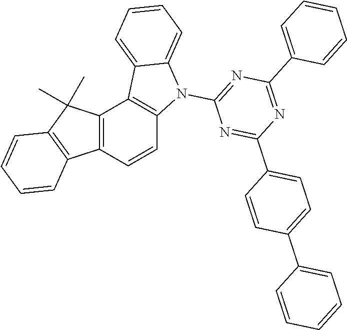 [SMILES: CC1(C)c2ccccc2-c2ccc3c(c21)c1ccccc1n3-c1nc(-c2ccccc2)nc(-c2ccc(-c3ccccc3)cc2)n1] | 801 |
| 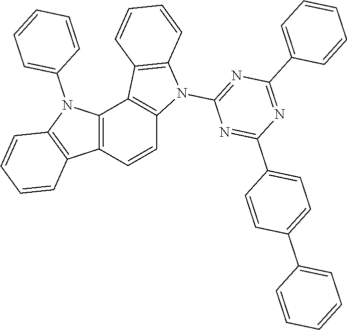 [SMILES: c1ccc(-c2ccc(-c3nc(-c4ccccc4)nc(-n4c5ccccc5c5c4ccc4c6ccccc6n(-c6ccccc6)c45)n3)cc2)cc1] | 802 |
| 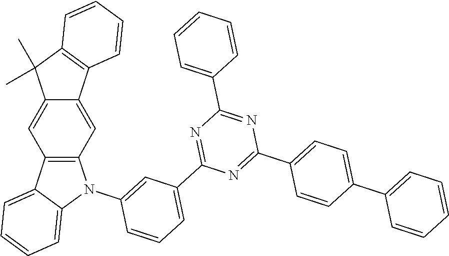 [SMILES: CC1(C)c2ccccc2-c2cc3c(cc21)c1ccccc1n3-c1cccc(-c2nc(-c3ccccc3)nc(-c3ccc(-c4ccccc4)cc3)n2)c1] | 803 |
| 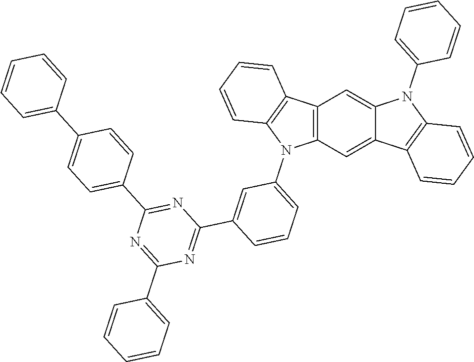 [SMILES: c1ccc(-c2ccc(-c3nc(-c4ccccc4)nc(-c4cccc(-n5c6ccccc6c6cc7c(cc65)c5ccccc5n7-c5ccccc5)c4)n3)cc2)cc1] | 804 |
| 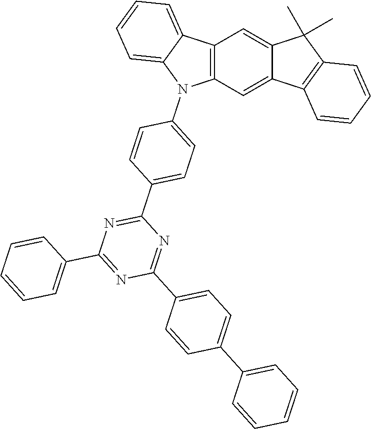 [SMILES: CC1(C)c2ccccc2-c2cc3c(cc21)c1ccccc1n3-c1ccc(-c2nc(-c3ccccc3)nc(-c3ccc(-c4ccccc4)cc3)n2)cc1] | 805 |
| 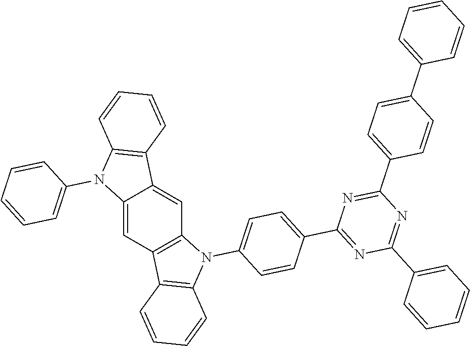 [SMILES: c1ccc(-c2ccc(-c3nc(-c4ccccc4)nc(-c4ccc(-n5c6ccccc6c6cc7c(cc65)c5ccccc5n7-c5ccccc5)cc4)n3)cc2)cc1] | 806 |
| 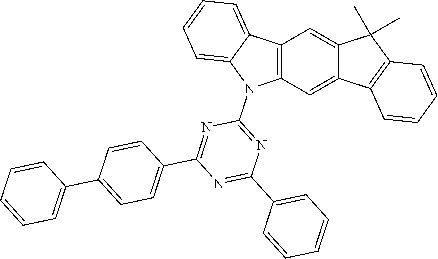 [SMILES: CC1(C)c2ccccc2-c2cc3c(cc21)c1ccccc1n3-c1nc(-c2ccccc2)nc(-c2ccc(-c3ccccc3)cc2)n1] | 807 |
| 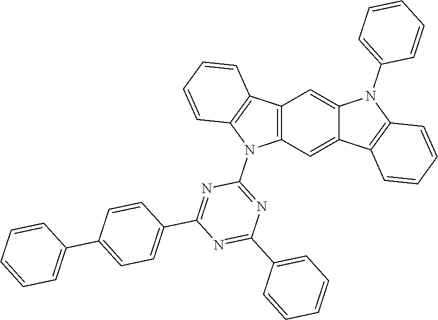 [SMILES: c1ccc(-c2ccc(-c3nc(-c4ccccc4)nc(-n4c5ccccc5c5cc6c(cc54)c4ccccc4n6-c4ccccc4)n3)cc2)cc1] | 808 |
| 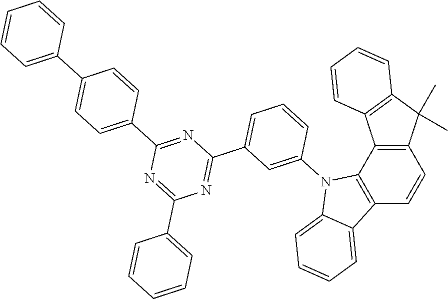 [SMILES: CC1(C)c2ccccc2-c2c1ccc1c3ccccc3n(-c3cccc(-c4nc(-c5ccccc5)nc(-c5ccc(-c6ccccc6)cc5)n4)c3)c21] | 809 |
| 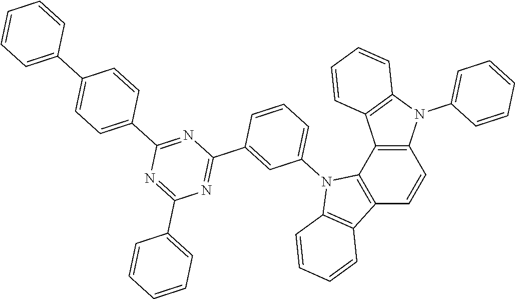 [SMILES: c1ccc(-c2ccc(-c3nc(-c4ccccc4)nc(-c4cccc(-n5c6ccccc6c6ccc7c(c8ccccc8n7-c7ccccc7)c65)c4)n3)cc2)cc1] | 810 |
| 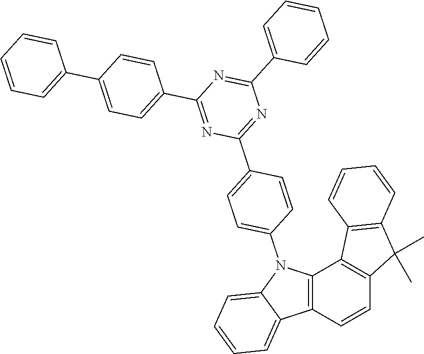 [SMILES: CC1(C)c2ccccc2-c2c1ccc1c3ccccc3n(-c3ccc(-c4nc(-c5ccccc5)nc(-c5ccc(-c6ccccc6)cc5)n4)cc3)c21] | 811 |
| 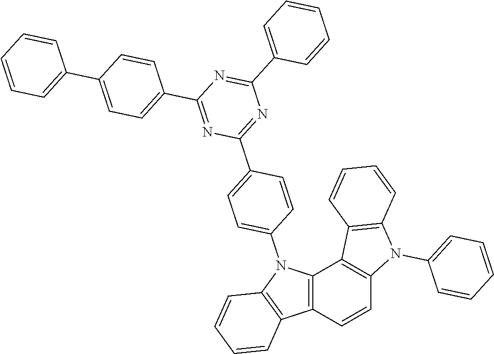 [SMILES: c1ccc(-c2ccc(-c3nc(-c4ccccc4)nc(-c4ccc(-n5c6ccccc6c6ccc7c(c8ccccc8n7-c7ccccc7)c65)cc4)n3)cc2)cc1] | 812 |
| 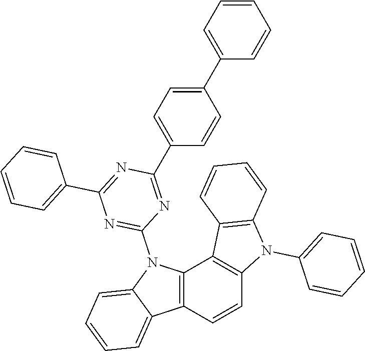 [SMILES: c1ccc(-c2ccc(-c3nc(-c4ccccc4)nc(-n4c5ccccc5c5ccc6c(c7ccccc7n6-c6ccccc6)c54)n3)cc2)cc1] | 813 |
| 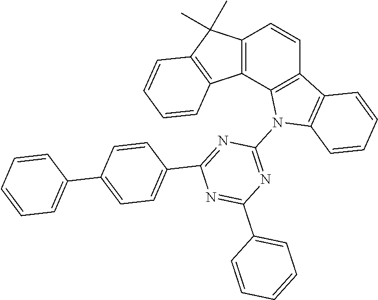 [SMILES: CC1(C)c2ccccc2-c2c1ccc1c3ccccc3n(-c3nc(-c4ccccc4)nc(-c4ccc(-c5ccccc5)cc4)n3)c21] | 814 |
| 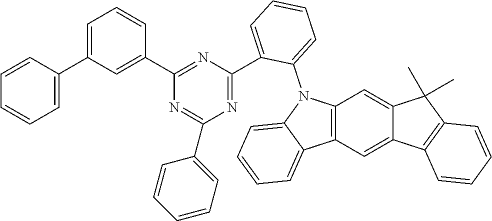 [SMILES: CC1(C)c2ccccc2-c2cc3c4ccccc4n(-c4ccccc4-c4nc(-c5ccccc5)nc(-c5cccc(-c6ccccc6)c5)n4)c3cc21] | 815 |
| 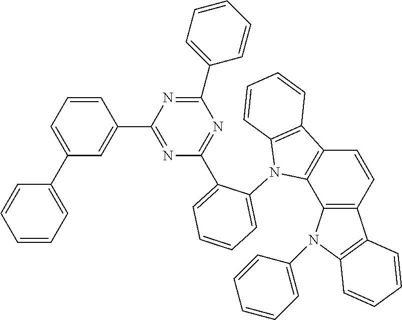 [SMILES: c1ccc(-c2cccc(-c3nc(-c4ccccc4)nc(-c4ccccc4-n4c5ccccc5c5ccc6c7ccccc7n(-c7ccccc7)c6c54)n3)c2)cc1] | 816 |
| 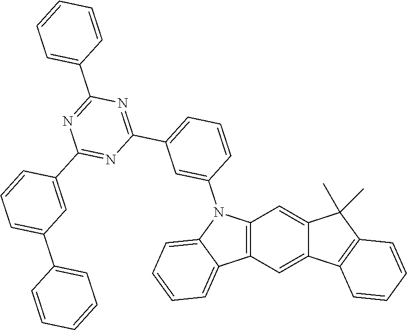 [SMILES: CC1(C)c2ccccc2-c2cc3c4ccccc4n(-c4cccc(-c5nc(-c6ccccc6)nc(-c6cccc(-c7ccccc7)c6)n5)c4)c3cc21] | 817 |
| 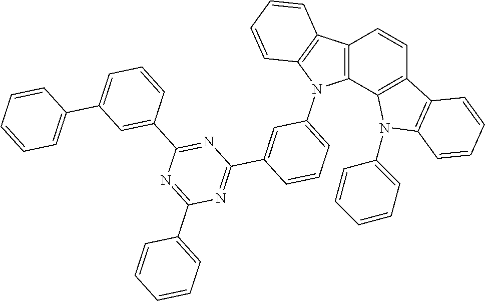 [SMILES: c1ccc(-c2cccc(-c3nc(-c4ccccc4)nc(-c4cccc(-n5c6ccccc6c6ccc7c8ccccc8n(-c8ccccc8)c7c65)c4)n3)c2)cc1] | 818 |
| 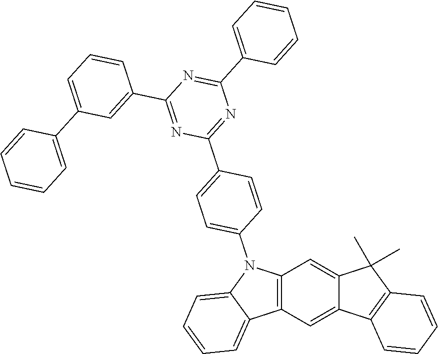 [SMILES: CC1(C)c2ccccc2-c2cc3c4ccccc4n(-c4ccc(-c5nc(-c6ccccc6)nc(-c6cccc(-c7ccccc7)c6)n5)cc4)c3cc21] | 819 |
| 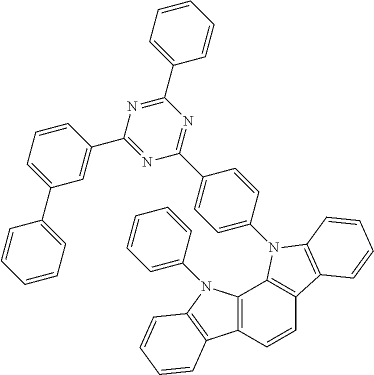 [SMILES: c1ccc(-c2cccc(-c3nc(-c4ccccc4)nc(-c4ccc(-n5c6ccccc6c6ccc7c8ccccc8n(-c8ccccc8)c7c65)cc4)n3)c2)cc1] | 820 |
| 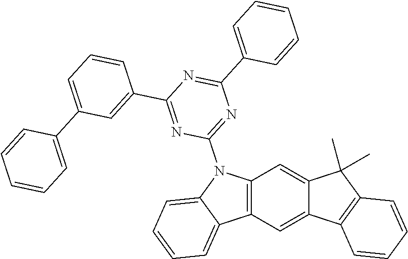 [SMILES: CC1(C)c2ccccc2-c2cc3c4ccccc4n(-c4nc(-c5ccccc5)nc(-c5cccc(-c6ccccc6)c5)n4)c3cc21] | 821 |
| 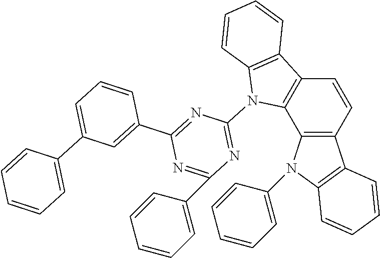 [SMILES: c1ccc(-c2cccc(-c3nc(-c4ccccc4)nc(-n4c5ccccc5c5ccc6c7ccccc7n(-c7ccccc7)c6c54)n3)c2)cc1] | 822 |
| 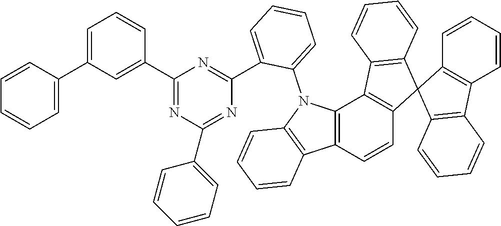 [SMILES: c1ccc(-c2cccc(-c3nc(-c4ccccc4)nc(-c4ccccc4-n4c5ccccc5c5ccc6c(c54)-c4ccccc4C64c5ccccc5-c5ccccc54)n3)c2)cc1] | 823 |
| 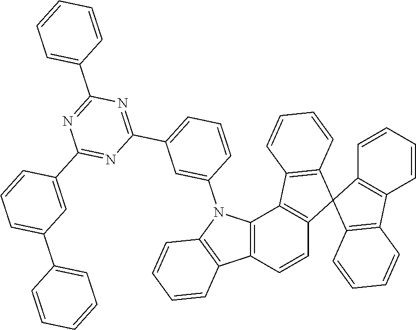 [SMILES: c1ccc(-c2cccc(-c3nc(-c4ccccc4)nc(-c4cccc(-n5c6ccccc6c6ccc7c(c65)-c5ccccc5C75c6ccccc6-c6ccccc65)c4)n3)c2)cc1] | 824 |
| 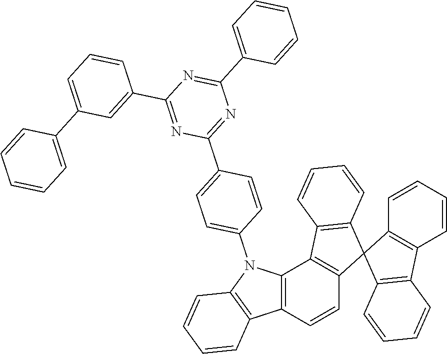 [SMILES: c1ccc(-c2cccc(-c3nc(-c4ccccc4)nc(-c4ccc(-n5c6ccccc6c6ccc7c(c65)-c5ccccc5C75c6ccccc6-c6ccccc65)cc4)n3)c2)cc1] | 825 |
| 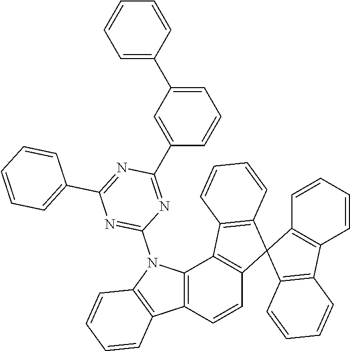 [SMILES: c1ccc(-c2cccc(-c3nc(-c4ccccc4)nc(-n4c5ccccc5c5ccc6c(c54)-c4ccccc4C64c5ccccc5-c5ccccc54)n3)c2)cc1] | 826 |
| 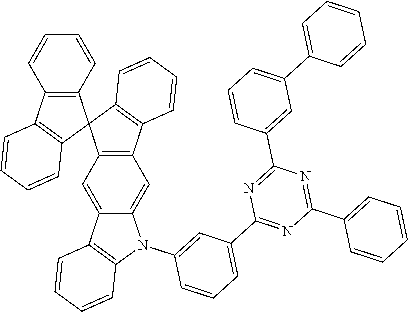 [SMILES: c1ccc(-c2cccc(-c3nc(-c4ccccc4)nc(-c4cccc(-n5c6ccccc6c6cc7c(cc65)-c5ccccc5C75c6ccccc6-c6ccccc65)c4)n3)c2)cc1] | 827 |
| 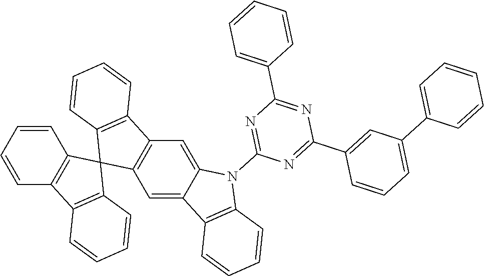 [SMILES: c1ccc(-c2cccc(-c3nc(-c4ccccc4)nc(-n4c5ccccc5c5cc6c(cc54)-c4ccccc4C64c5ccccc5-c5ccccc54)n3)c2)cc1] | 828 |
| 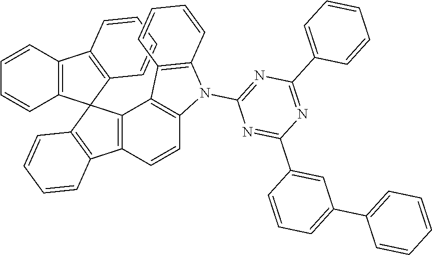 [SMILES: c1ccc(-c2cccc(-c3nc(-c4ccccc4)nc(-n4c5ccccc5c5c6c(ccc54)-c4ccccc4C64c5ccccc5-c5ccccc54)n3)c2)cc1] | 829 |
| 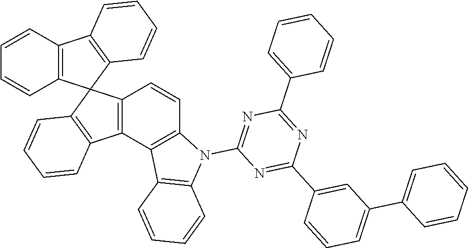 [SMILES: c1ccc(-c2cccc(-c3nc(-c4ccccc4)nc(-n4c5ccccc5c5c6c(ccc54)C4(c5ccccc5-c5ccccc54)c4ccccc4-6)n3)c2)cc1] | 830 |
| 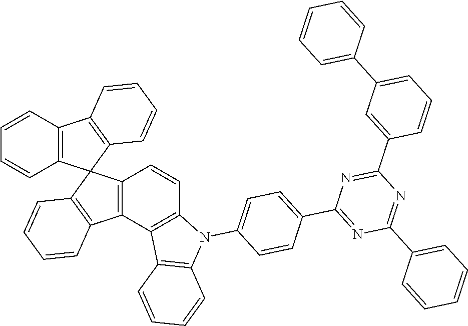 [SMILES: c1ccc(-c2cccc(-c3nc(-c4ccccc4)nc(-c4ccc(-n5c6ccccc6c6c7c(ccc65)C5(c6ccccc6-c6ccccc65)c5ccccc5-7)cc4)n3)c2)cc1] | 831 |
| 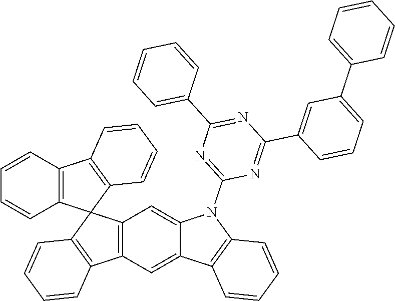 [SMILES: c1ccc(-c2cccc(-c3nc(-c4ccccc4)nc(-n4c5ccccc5c5cc6c(cc54)C4(c5ccccc5-c5ccccc54)c4ccccc4-6)n3)c2)cc1] | 832 |
| 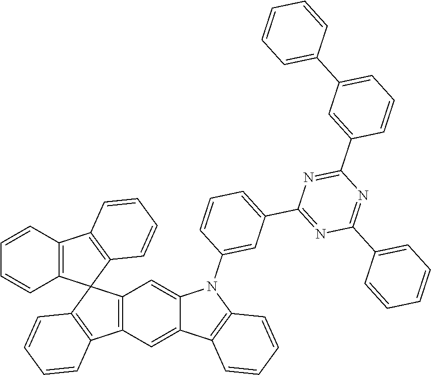 [SMILES: c1ccc(-c2cccc(-c3nc(-c4ccccc4)nc(-c4cccc(-n5c6ccccc6c6cc7c(cc65)C5(c6ccccc6-c6ccccc65)c5ccccc5-7)c4)n3)c2)cc1] | 833 |
| 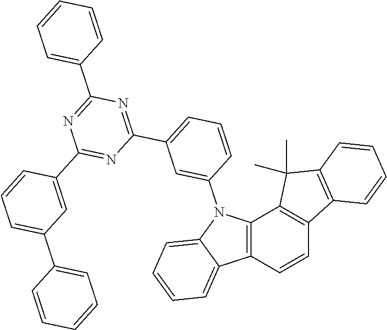 [SMILES: CC1(C)c2ccccc2-c2ccc3c4ccccc4n(-c4cccc(-c5nc(-c6ccccc6)nc(-c6cccc(-c7ccccc7)c6)n5)c4)c3c21] | 834 |
| 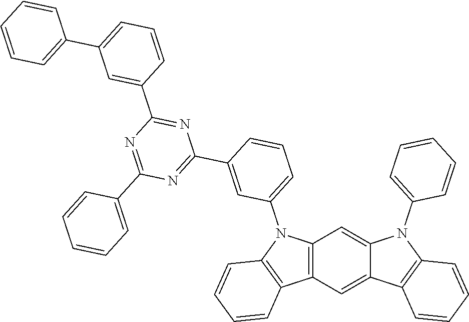 [SMILES: c1ccc(-c2cccc(-c3nc(-c4ccccc4)nc(-c4cccc(-n5c6ccccc6c6cc7c8ccccc8n(-c8ccccc8)c7cc65)c4)n3)c2)cc1] | 835 |
| 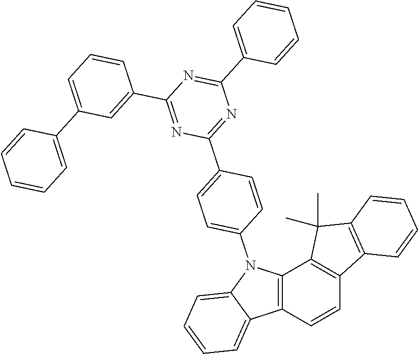 [SMILES: CC1(C)c2ccccc2-c2ccc3c4ccccc4n(-c4ccc(-c5nc(-c6ccccc6)nc(-c6cccc(-c7ccccc7)c6)n5)cc4)c3c21] | 836 |
| 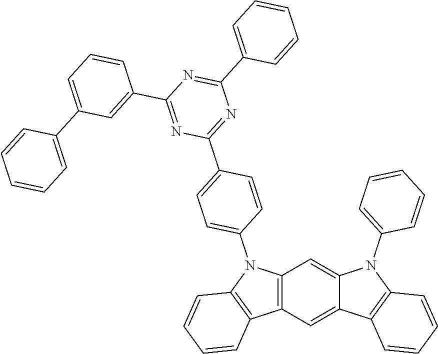 [SMILES: c1ccc(-c2cccc(-c3nc(-c4ccccc4)nc(-c4ccc(-n5c6ccccc6c6cc7c8ccccc8n(-c8ccccc8)c7cc65)cc4)n3)c2)cc1] | 837 |
| 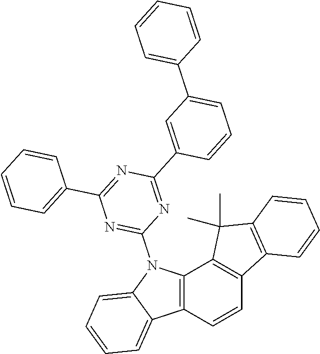 [SMILES: CC1(C)c2ccccc2-c2ccc3c4ccccc4n(-c4nc(-c5ccccc5)nc(-c5cccc(-c6ccccc6)c5)n4)c3c21] | 838 |
| 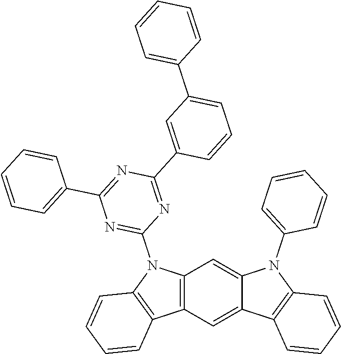 [SMILES: c1ccc(-c2cccc(-c3nc(-c4ccccc4)nc(-n4c5ccccc5c5cc6c7ccccc7n(-c7ccccc7)c6cc54)n3)c2)cc1] | 839 |
| 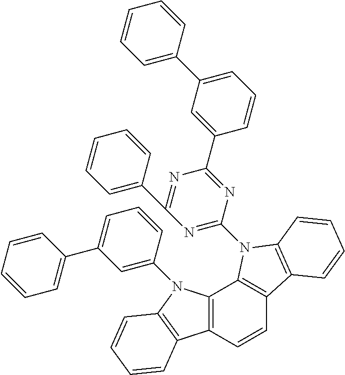 [SMILES: c1ccc(-c2cccc(-c3nc(-c4ccccc4)nc(-n4c5ccccc5c5ccc6c7ccccc7n(-c7cccc(-c8ccccc8)c7)c6c54)n3)c2)cc1] | 840 |
| 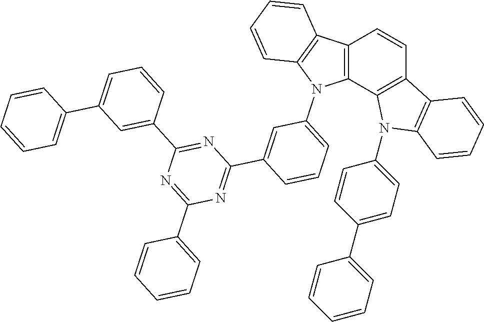 [SMILES: c1ccc(-c2ccc(-n3c4ccccc4c4ccc5c6ccccc6n(-c6cccc(-c7nc(-c8ccccc8)nc(-c8cccc(-c9ccccc9)c8)n7)c6)c5c43)cc2)cc1] | 841 |
| 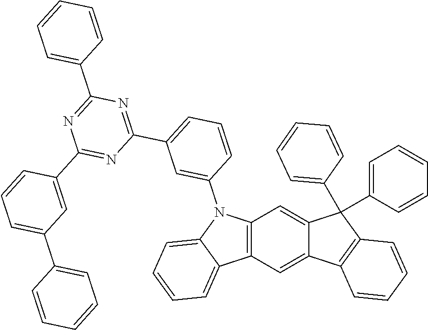 [SMILES: c1ccc(-c2cccc(-c3nc(-c4ccccc4)nc(-c4cccc(-n5c6ccccc6c6cc7c(cc65)C(c5ccccc5)(c5ccccc5)c5ccccc5-7)c4)n3)c2)cc1] | 842 |
| 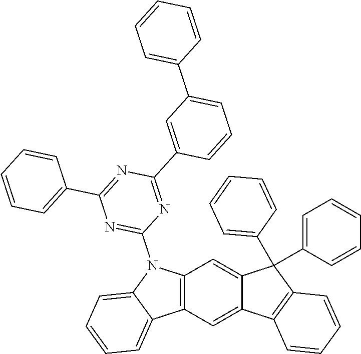 [SMILES: c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1] | 843 |
| 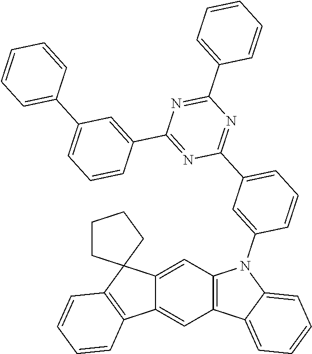 [SMILES: c1ccc(-c2cccc(-c3nc(-c4ccccc4)nc(-c4cccc(-n5c6ccccc6c6cc7c(cc65)C5(CCCC5)c5ccccc5-7)c4)n3)c2)cc1] | 844 |
| 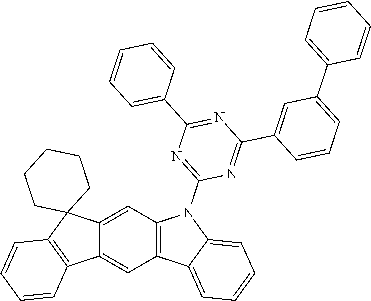 [SMILES: c1ccc(-c2cccc(-c3nc(-c4ccccc4)nc(-n4c5ccccc5c5cc6c(cc54)C4(CCCCC4)c4ccccc4-6)n3)c2)cc1] | 845 |
| 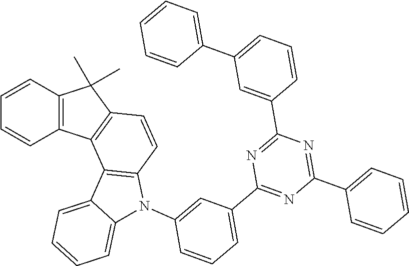 [SMILES: CC1(C)c2ccccc2-c2c1ccc1c2c2ccccc2n1-c1cccc(-c2nc(-c3ccccc3)nc(-c3cccc(-c4ccccc4)c3)n2)c1] | 846 |
| 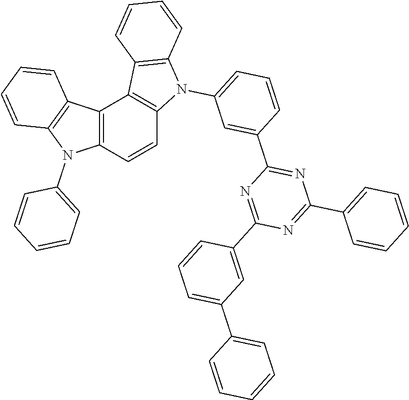 [SMILES: c1ccc(-c2cccc(-c3nc(-c4ccccc4)nc(-c4cccc(-n5c6ccccc6c6c7c8ccccc8n(-c8ccccc8)c7ccc65)c4)n3)c2)cc1] | 847 |
| 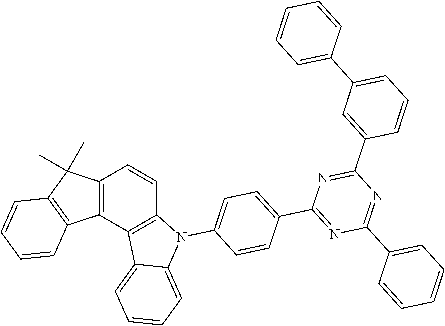 [SMILES: CC1(C)c2ccccc2-c2c1ccc1c2c2ccccc2n1-c1ccc(-c2nc(-c3ccccc3)nc(-c3cccc(-c4ccccc4)c3)n2)cc1] | 848 |
| 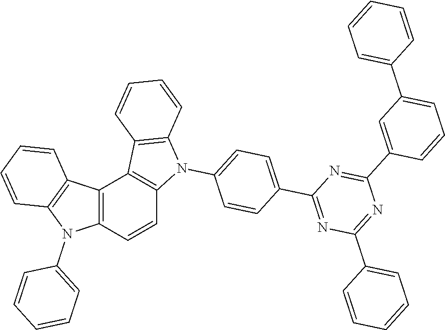 [SMILES: c1ccc(-c2cccc(-c3nc(-c4ccccc4)nc(-c4ccc(-n5c6ccccc6c6c7c8ccccc8n(-c8ccccc8)c7ccc65)cc4)n3)c2)cc1] | 849 |
| 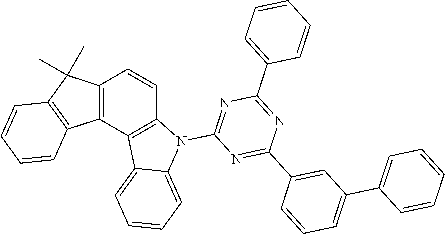 [SMILES: CC1(C)c2ccccc2-c2c1ccc1c2c2ccccc2n1-c1nc(-c2ccccc2)nc(-c2cccc(-c3ccccc3)c2)n1] | 850 |
| 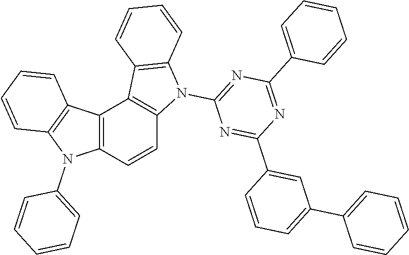 [SMILES: c1ccc(-c2cccc(-c3nc(-c4ccccc4)nc(-n4c5ccccc5c5c6c7ccccc7n(-c7ccccc7)c6ccc54)n3)c2)cc1] | 851 |
| 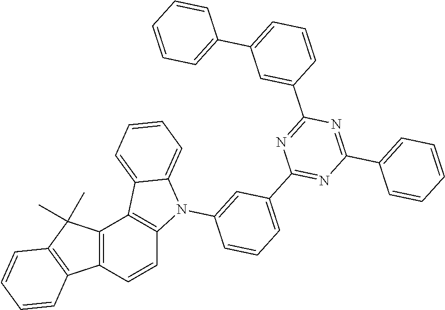 [SMILES: CC1(C)c2ccccc2-c2ccc3c(c21)c1ccccc1n3-c1cccc(-c2nc(-c3ccccc3)nc(-c3cccc(-c4ccccc4)c3)n2)c1] | 852 |
| 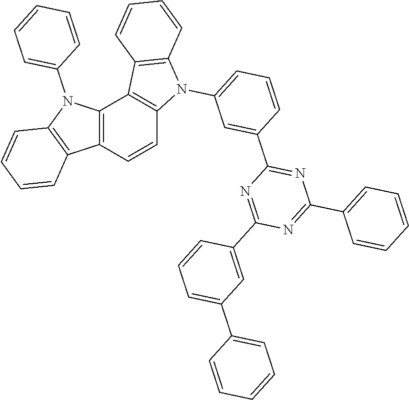 [SMILES: c1ccc(-c2cccc(-c3nc(-c4ccccc4)nc(-c4cccc(-n5c6ccccc6c6c5ccc5c7ccccc7n(-c7ccccc7)c56)c4)n3)c2)cc1] | 853 |
| 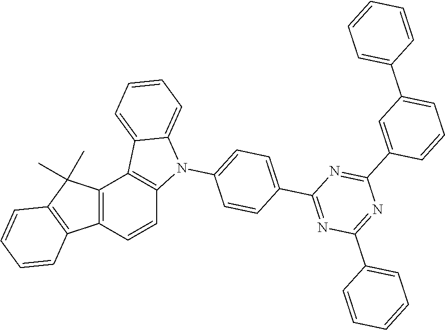 [SMILES: CC1(C)c2ccccc2-c2ccc3c(c21)c1ccccc1n3-c1ccc(-c2nc(-c3ccccc3)nc(-c3cccc(-c4ccccc4)c3)n2)cc1] | 854 |
| 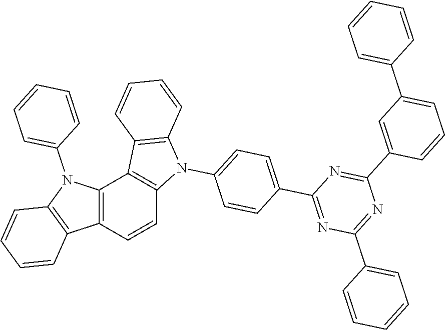 [SMILES: c1ccc(-c2cccc(-c3nc(-c4ccccc4)nc(-c4ccc(-n5c6ccccc6c6c5ccc5c7ccccc7n(-c7ccccc7)c56)cc4)n3)c2)cc1] | 855 |
| 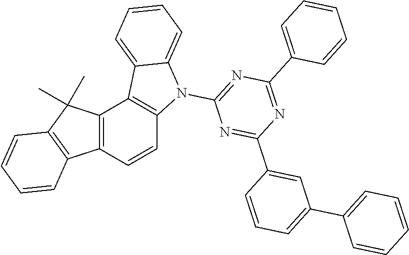 [SMILES: CC1(C)c2ccccc2-c2ccc3c(c21)c1ccccc1n3-c1nc(-c2ccccc2)nc(-c2cccc(-c3ccccc3)c2)n1] | 856 |
| 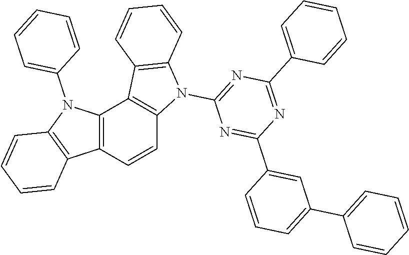 [SMILES: c1ccc(-c2cccc(-c3nc(-c4ccccc4)nc(-n4c5ccccc5c5c4ccc4c6ccccc6n(-c6ccccc6)c45)n3)c2)cc1] | 857 |
| 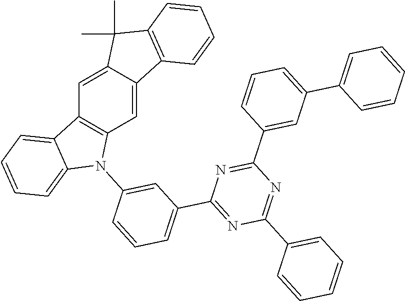 [SMILES: CC1(C)c2ccccc2-c2cc3c(cc21)c1ccccc1n3-c1cccc(-c2nc(-c3ccccc3)nc(-c3cccc(-c4ccccc4)c3)n2)c1] | 858 |
| 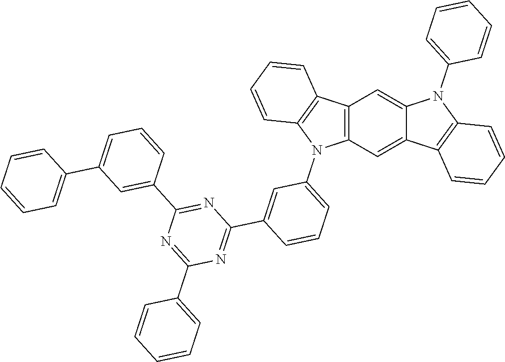 [SMILES: c1ccc(-c2cccc(-c3nc(-c4ccccc4)nc(-c4cccc(-n5c6ccccc6c6cc7c(cc65)c5ccccc5n7-c5ccccc5)c4)n3)c2)cc1] | 859 |
| 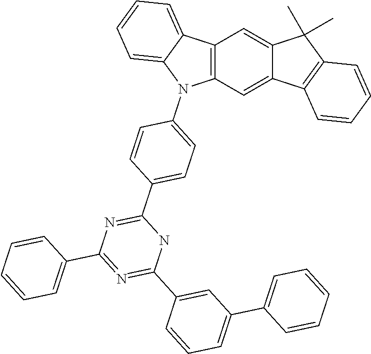 | 860 |
| 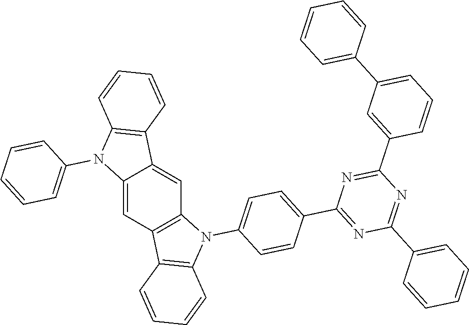 [SMILES: c1ccc(-c2cccc(-c3nc(-c4ccccc4)nc(-c4ccc(-n5c6ccccc6c6cc7c(cc65)c5ccccc5n7-c5ccccc5)cc4)n3)c2)cc1] | 861 |
| 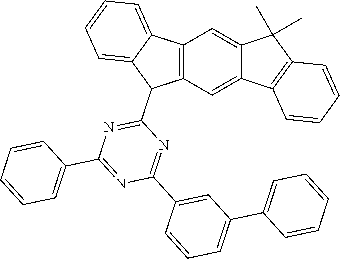 [SMILES: CC1(C)c2ccccc2-c2cc3c(cc21)-c1ccccc1C3c1nc(-c2ccccc2)nc(-c2cccc(-c3ccccc3)c2)n1] | 862 |
| 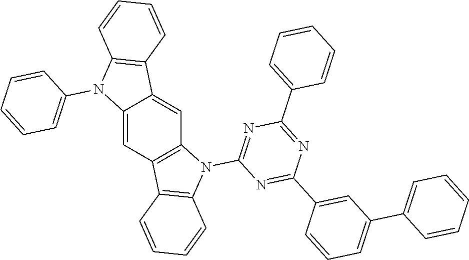 [SMILES: c1ccc(-c2cccc(-c3nc(-c4ccccc4)nc(-n4c5ccccc5c5cc6c(cc54)c4ccccc4n6-c4ccccc4)n3)c2)cc1] | 863 |
| 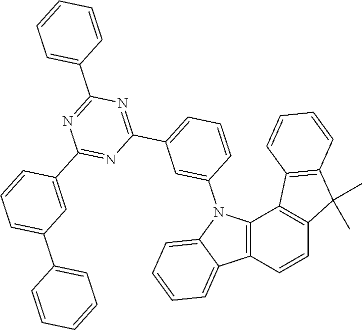 [SMILES: CC1(C)c2ccccc2-c2c1ccc1c3ccccc3n(-c3cccc(-c4nc(-c5ccccc5)nc(-c5cccc(-c6ccccc6)c5)n4)c3)c21] | 864 |
| 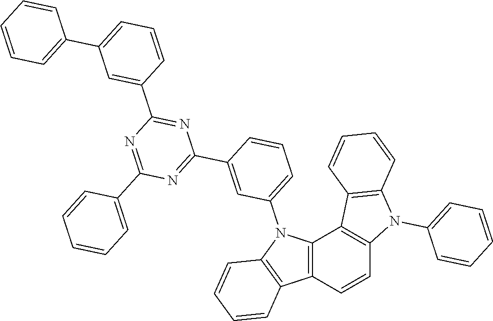 [SMILES: c1ccc(-c2cccc(-c3nc(-c4ccccc4)nc(-c4cccc(-n5c6ccccc6c6ccc7c(c8ccccc8n7-c7ccccc7)c65)c4)n3)c2)cc1] | 865 |
| 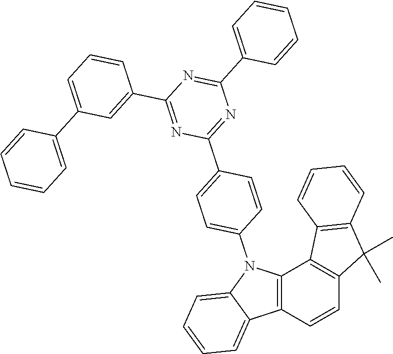 [SMILES: CC1(C)c2ccccc2-c2c1ccc1c3ccccc3n(-c3ccc(-c4nc(-c5ccccc5)nc(-c5cccc(-c6ccccc6)c5)n4)cc3)c21] | 866 |
| 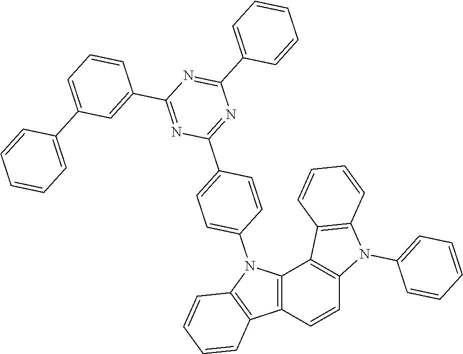 [SMILES: c1ccc(-c2cccc(-c3nc(-c4ccccc4)nc(-c4ccc(-n5c6ccccc6c6ccc7c(c8ccccc8n7-c7ccccc7)c65)cc4)n3)c2)cc1] | 867 |
| 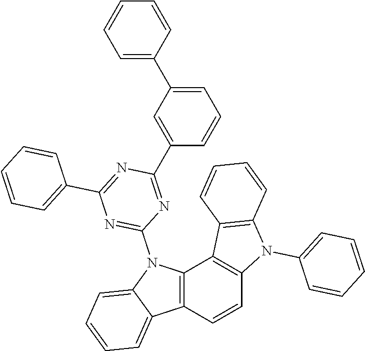 [SMILES: c1ccc(-c2cccc(-c3nc(-c4ccccc4)nc(-n4c5ccccc5c5ccc6c(c7ccccc7n6-c6ccccc6)c54)n3)c2)cc1] | 868 |
| 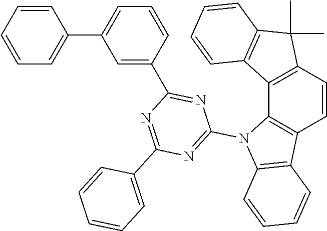 [SMILES: CC1(C)c2ccccc2-c2c1ccc1c3ccccc3n(-c3nc(-c4ccccc4)nc(-c4cccc(-c5ccccc5)c4)n3)c21] | 869 |
| 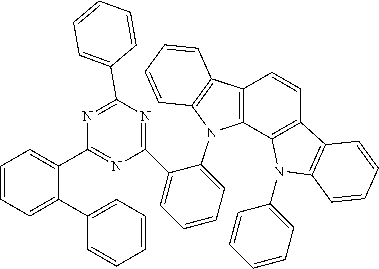 [SMILES: c1ccc(-c2nc(-c3ccccc3-c3ccccc3)nc(-c3ccccc3-n3c4ccccc4c4ccc5c6ccccc6n(-c6ccccc6)c5c43)n2)cc1] | 870 |
| 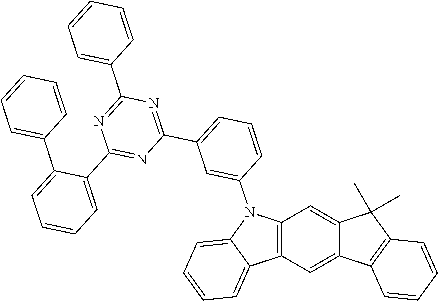 [SMILES: CC1(C)c2ccccc2-c2cc3c4ccccc4n(-c4cccc(-c5nc(-c6ccccc6)nc(-c6ccccc6-c6ccccc6)n5)c4)c3cc21] | 871 |
| 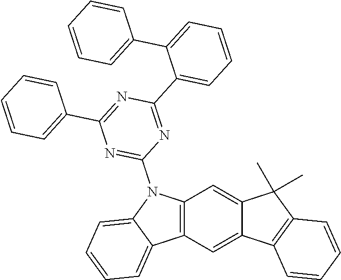 [SMILES: CC1(C)c2ccccc2-c2cc3c4ccccc4n(-c4nc(-c5ccccc5)nc(-c5ccccc5-c5ccccc5)n4)c3cc21] | 872 |
| 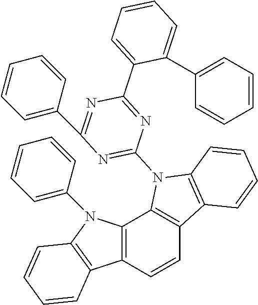 [SMILES: c1ccc(-c2nc(-c3ccccc3-c3ccccc3)nc(-n3c4ccccc4c4ccc5c6ccccc6n(-c6ccccc6)c5c43)n2)cc1] | 873 |
| 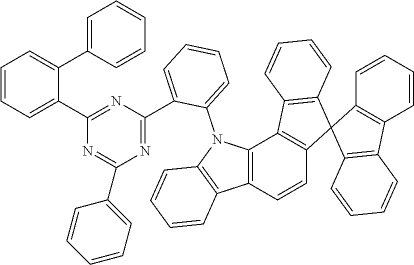 [SMILES: c1ccc(-c2nc(-c3ccccc3-c3ccccc3)nc(-c3ccccc3-n3c4ccccc4c4ccc5c(c43)-c3ccccc3C53c4ccccc4-c4ccccc43)n2)cc1] | 874 |
| 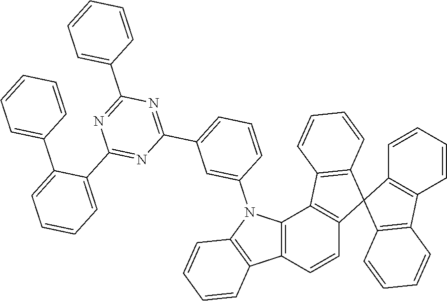 [SMILES: c1ccc(-c2nc(-c3cccc(-n4c5ccccc5c5ccc6c(c54)-c4ccccc4C64c5ccccc5-c5ccccc54)c3)nc(-c3ccccc3-c3ccccc3)n2)cc1] | 875 |
| 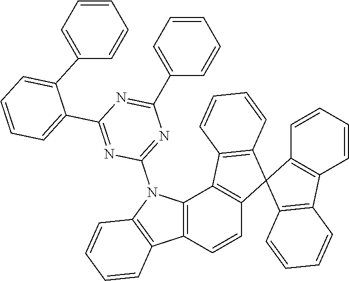 [SMILES: c1ccc(-c2nc(-c3ccccc3-c3ccccc3)nc(-n3c4ccccc4c4ccc5c(c43)-c3ccccc3C53c4ccccc4-c4ccccc43)n2)cc1] | 876 |
| 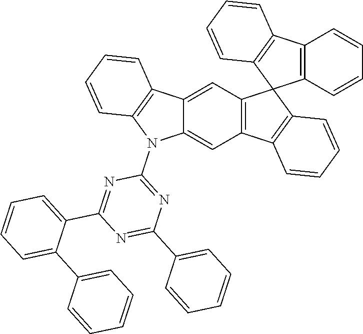 [SMILES: c1ccc(-c2nc(-c3ccccc3-c3ccccc3)nc(-n3c4ccccc4c4cc5c(cc43)-c3ccccc3C53c4ccccc4-c4ccccc43)n2)cc1] | 877 |
| 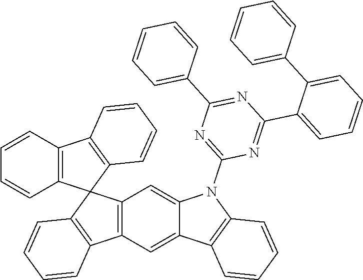 [SMILES: c1ccc(-c2nc(-c3ccccc3-c3ccccc3)nc(-n3c4ccccc4c4cc5c(cc43)C3(c4ccccc4-c4ccccc43)c3ccccc3-5)n2)cc1] | 878 |
| 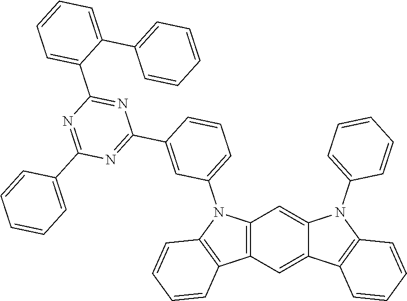 [SMILES: c1ccc(-c2nc(-c3cccc(-n4c5ccccc5c5cc6c7ccccc7n(-c7ccccc7)c6cc54)c3)nc(-c3ccccc3-c3ccccc3)n2)cc1] | 879 |
| 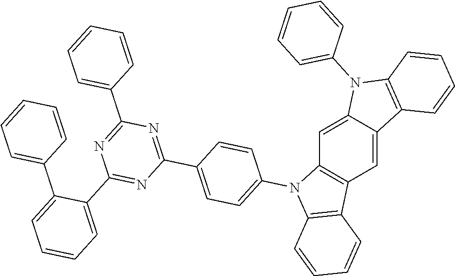 [SMILES: c1ccc(-c2nc(-c3ccc(-n4c5ccccc5c5cc6c7ccccc7n(-c7ccccc7)c6cc54)cc3)nc(-c3ccccc3-c3ccccc3)n2)cc1] | 880 |
| 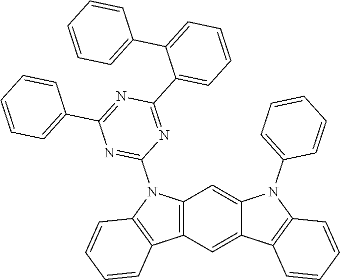 [SMILES: c1ccc(-c2nc(-c3ccccc3-c3ccccc3)nc(-n3c4ccccc4c4cc5c6ccccc6n(-c6ccccc6)c5cc43)n2)cc1] | 881 |
| 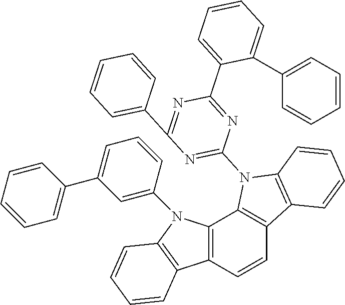 [SMILES: c1ccc(-c2cccc(-n3c4ccccc4c4ccc5c6ccccc6n(-c6nc(-c7ccccc7)nc(-c7ccccc7-c7ccccc7)n6)c5c43)c2)cc1] | 882 |
| 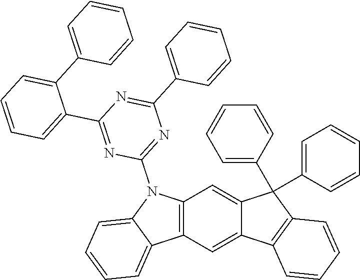 [SMILES: c1ccc(-c2nc(-c3ccccc3-c3ccccc3)nc(-n3c4ccccc4c4cc5c(cc43)C(c3ccccc3)(c3ccccc3)c3ccccc3-5)n2)cc1] | 883 |
| 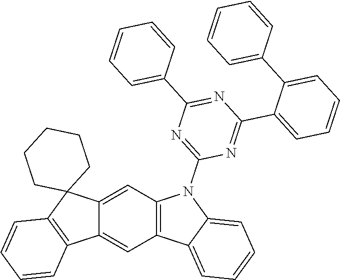 [SMILES: c1ccc(-c2nc(-c3ccccc3-c3ccccc3)nc(-n3c4ccccc4c4cc5c(cc43)C3(CCCCC3)c3ccccc3-5)n2)cc1] | 884 |
| 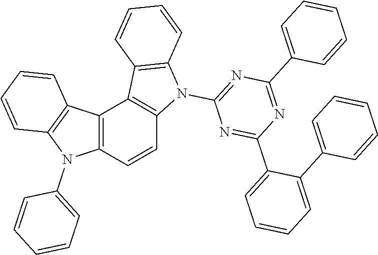 [SMILES: c1ccc(-c2nc(-c3ccccc3-c3ccccc3)nc(-n3c4ccccc4c4c5c6ccccc6n(-c6ccccc6)c5ccc43)n2)cc1] | 885 |
| 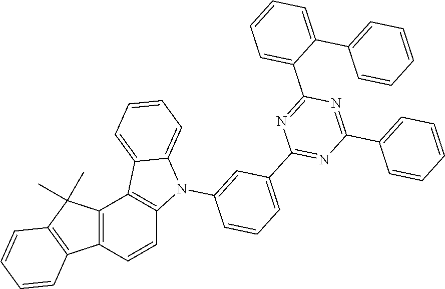 [SMILES: CC1(C)c2ccccc2-c2ccc3c(c21)c1ccccc1n3-c1cccc(-c2nc(-c3ccccc3)nc(-c3ccccc3-c3ccccc3)n2)c1] | 886 |
| 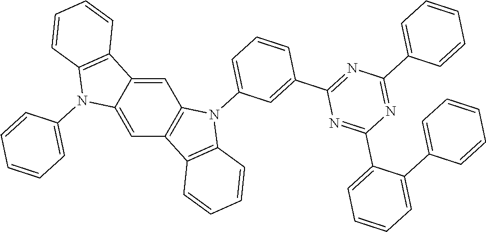 [SMILES: c1ccc(-c2nc(-c3cccc(-n4c5ccccc5c5cc6c(cc54)c4ccccc4n6-c4ccccc4)c3)nc(-c3ccccc3-c3ccccc3)n2)cc1] | 887 |
| 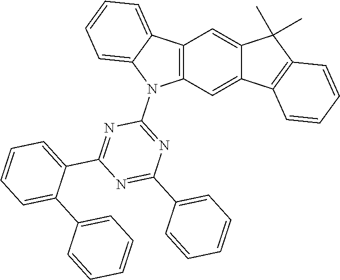 [SMILES: CC1(C)c2ccccc2-c2cc3c(cc21)c1ccccc1n3-c1nc(-c2ccccc2)nc(-c2ccccc2-c2ccccc2)n1] | 888 |
| 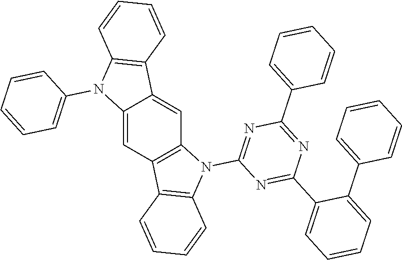 [SMILES: c1ccc(-c2nc(-c3ccccc3-c3ccccc3)nc(-n3c4ccccc4c4cc5c(cc43)c3ccccc3n5-c3ccccc3)n2)cc1] | 889 |
| 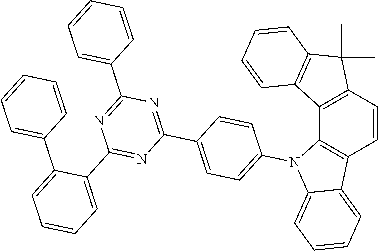 [SMILES: CC1(C)c2ccccc2-c2c1ccc1c3ccccc3n(-c3ccc(-c4nc(-c5ccccc5)nc(-c5ccccc5-c5ccccc5)n4)cc3)c21] | 890 |
| 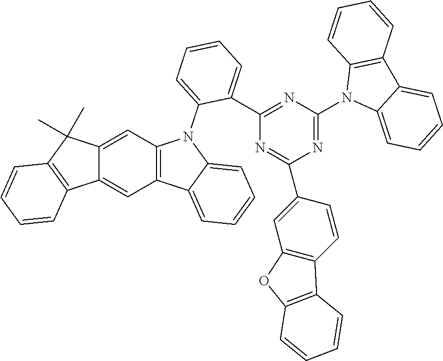 [SMILES: CC1(C)c2ccccc2-c2cc3c4ccccc4n(-c4ccccc4-c4nc(-c5ccc6c(c5)oc5ccccc56)nc(-n5c6ccccc6c6ccccc65)n4)c3cc21] | 891 |
| 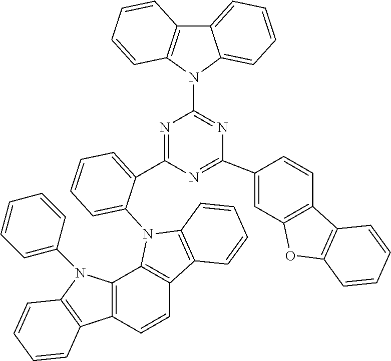 [SMILES: c1ccc(-n2c3ccccc3c3ccc4c5ccccc5n(-c5ccccc5-c5nc(-c6ccc7c(c6)oc6ccccc67)nc(-n6c7ccccc7c7ccccc76)n5)c4c32)cc1] | 892 |
| 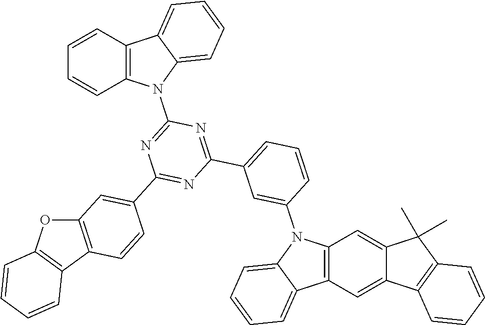 [SMILES: CC1(C)c2ccccc2-c2cc3c4ccccc4n(-c4cccc(-c5nc(-c6ccc7c(c6)oc6ccccc67)nc(-n6c7ccccc7c7ccccc76)n5)c4)c3cc21] | 893 |
| 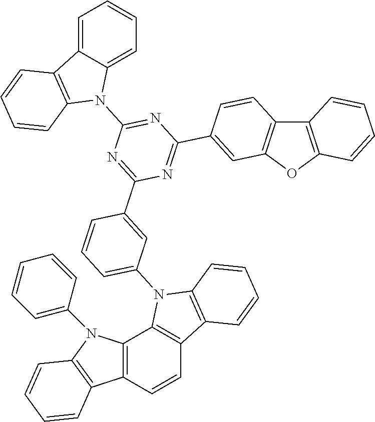 [SMILES: c1ccc(-n2c3ccccc3c3ccc4c5ccccc5n(-c5cccc(-c6nc(-c7ccc8c(c7)oc7ccccc78)nc(-n7c8ccccc8c8ccccc87)n6)c5)c4c32)cc1] | 894 |
| 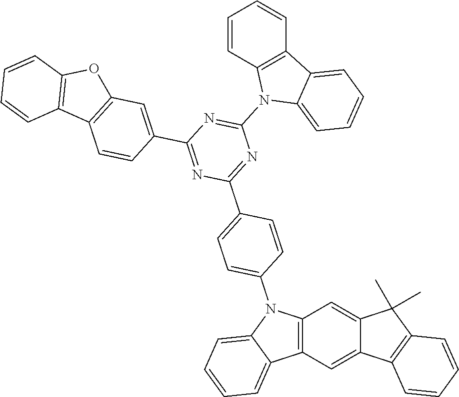 [SMILES: CC1(C)c2ccccc2-c2cc3c4ccccc4n(-c4ccc(-c5nc(-c6ccc7c(c6)oc6ccccc67)nc(-n6c7ccccc7c7ccccc76)n5)cc4)c3cc21] | 895 |
| 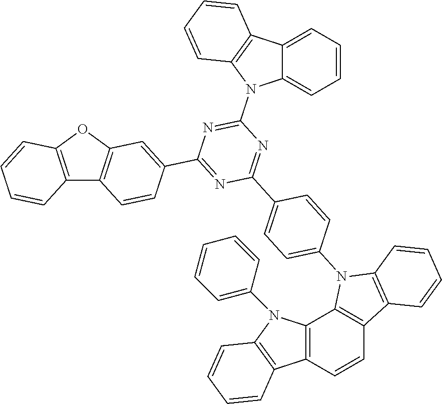 [SMILES: c1ccc(-n2c3ccccc3c3ccc4c5ccccc5n(-c5ccc(-c6nc(-c7ccc8c(c7)oc7ccccc78)nc(-n7c8ccccc8c8ccccc87)n6)cc5)c4c32)cc1] | 896 |
| 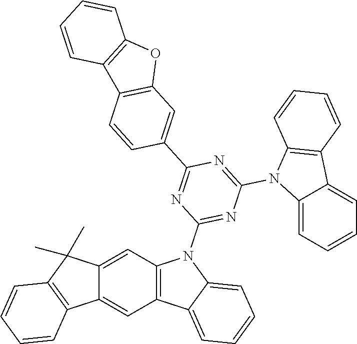 [SMILES: CC1(C)c2ccccc2-c2cc3c4ccccc4n(-c4nc(-c5ccc6c(c5)oc5ccccc56)nc(-n5c6ccccc6c6ccccc65)n4)c3cc21] | 897 |
| 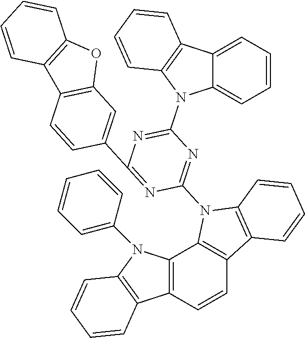 [SMILES: c1ccc(-n2c3ccccc3c3ccc4c5ccccc5n(-c5nc(-c6ccc7c(c6)oc6ccccc67)nc(-n6c7ccccc7c7ccccc76)n5)c4c32)cc1] | 898 |
| 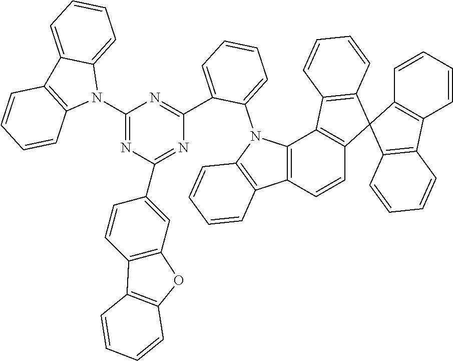 [SMILES: c1ccc(-n2c3ccccc3c3ccc4c(c32)-c2ccccc2C42c3ccccc3-c3ccccc32)c(-c2nc(-c3ccc4c(c3)oc3ccccc34)nc(-n3c4ccccc4c4ccccc43)n2)c1] | 899 |
| 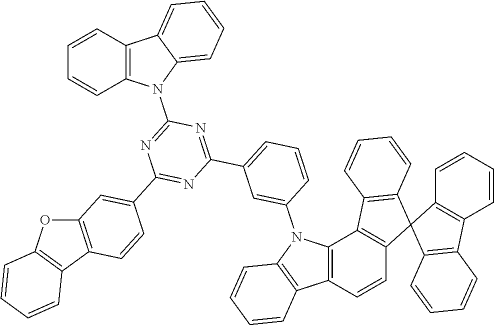 [SMILES: c1cc(-c2nc(-c3ccc4c(c3)oc3ccccc34)nc(-n3c4ccccc4c4ccccc43)n2)cc(-n2c3ccccc3c3ccc4c(c32)-c2ccccc2C42c3ccccc3-c3ccccc32)c1] | 900 |
| 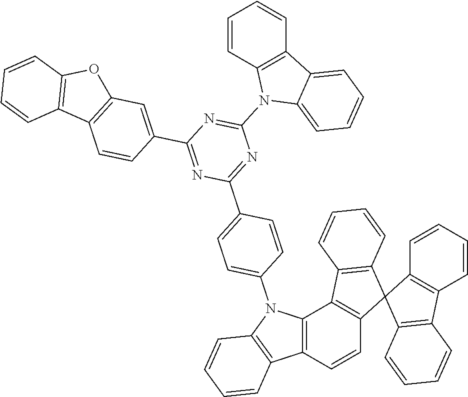 [SMILES: c1ccc2c(c1)-c1ccccc1C21c2ccccc2-c2c1ccc1c3ccccc3n(-c3ccc(-c4nc(-c5ccc6c(c5)oc5ccccc56)nc(-n5c6ccccc6c6ccccc65)n4)cc3)c21] | 901 |
| 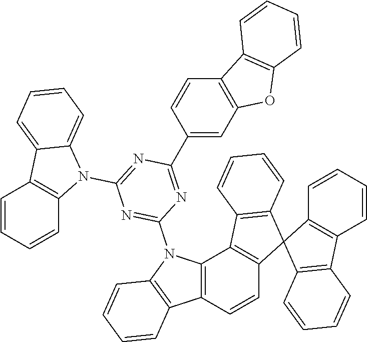 [SMILES: c1ccc2c(c1)-c1ccccc1C21c2ccccc2-c2c1ccc1c3ccccc3n(-c3nc(-c4ccc5c(c4)oc4ccccc45)nc(-n4c5ccccc5c5ccccc54)n3)c21] | 902 |
| 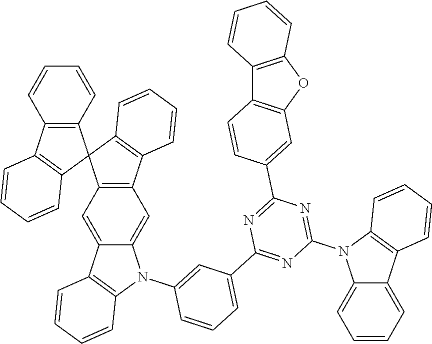 [SMILES: c1cc(-c2nc(-c3ccc4c(c3)oc3ccccc34)nc(-n3c4ccccc4c4ccccc43)n2)cc(-n2c3ccccc3c3cc4c(cc32)-c2ccccc2C42c3ccccc3-c3ccccc32)c1] | 903 |
| 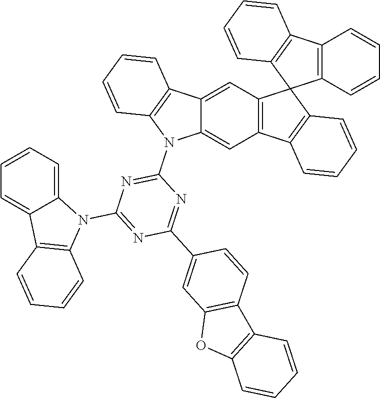 [SMILES: c1ccc2c(c1)-c1ccccc1C21c2ccccc2-c2cc3c(cc21)c1ccccc1n3-c1nc(-c2ccc3c(c2)oc2ccccc23)nc(-n2c3ccccc3c3ccccc32)n1] | 904 |
| 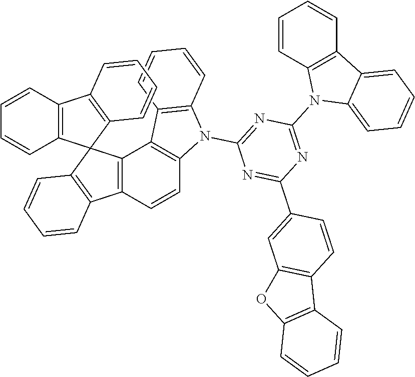 [SMILES: c1ccc2c(c1)-c1ccccc1C21c2ccccc2-c2ccc3c(c21)c1ccccc1n3-c1nc(-c2ccc3c(c2)oc2ccccc23)nc(-n2c3ccccc3c3ccccc32)n1] | 905 |
| 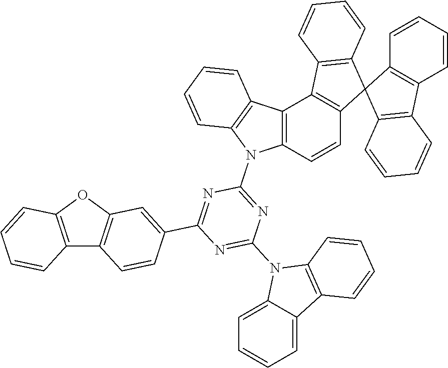 [SMILES: c1ccc2c(c1)-c1ccccc1C21c2ccccc2-c2c1ccc1c2c2ccccc2n1-c1nc(-c2ccc3c(c2)oc2ccccc23)nc(-n2c3ccccc3c3ccccc32)n1] | 906 |
| 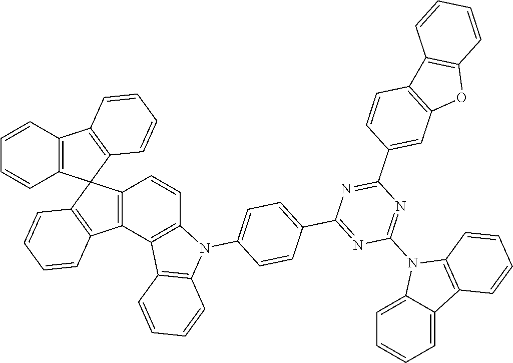 [SMILES: c1ccc2c(c1)-c1ccccc1C21c2ccccc2-c2c1ccc1c2c2ccccc2n1-c1ccc(-c2nc(-c3ccc4c(c3)oc3ccccc34)nc(-n3c4ccccc4c4ccccc43)n2)cc1] | 907 |
| 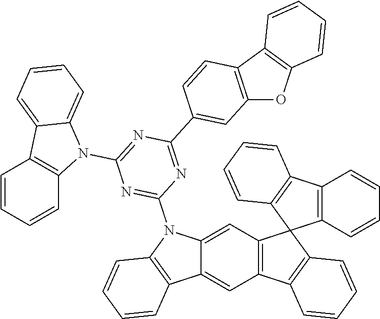 [SMILES: c1ccc2c(c1)-c1ccccc1C21c2ccccc2-c2cc3c4ccccc4n(-c4nc(-c5ccc6c(c5)oc5ccccc56)nc(-n5c6ccccc6c6ccccc65)n4)c3cc21] | 908 |
| 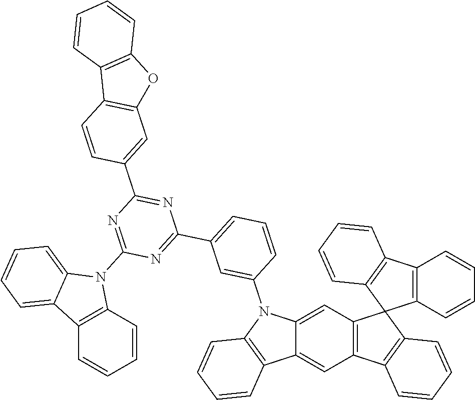 [SMILES: c1cc(-c2nc(-c3ccc4c(c3)oc3ccccc34)nc(-n3c4ccccc4c4ccccc43)n2)cc(-n2c3ccccc3c3cc4c(cc32)C2(c3ccccc3-c3ccccc32)c2ccccc2-4)c1] | 909 |
| 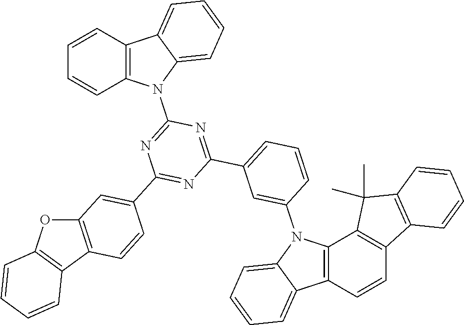 [SMILES: CC1(C)c2ccccc2-c2ccc3c4ccccc4n(-c4cccc(-c5nc(-c6ccc7c(c6)oc6ccccc67)nc(-n6c7ccccc7c7ccccc76)n5)c4)c3c21] | 910 |
| 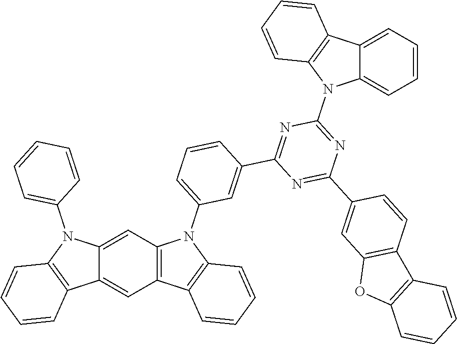 [SMILES: c1ccc(-n2c3ccccc3c3cc4c5ccccc5n(-c5cccc(-c6nc(-c7ccc8c(c7)oc7ccccc78)nc(-n7c8ccccc8c8ccccc87)n6)c5)c4cc32)cc1] | 911 |
| 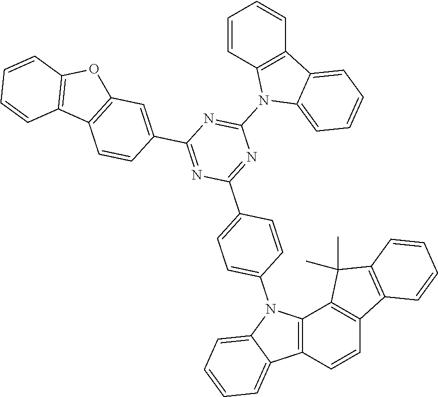 [SMILES: CC1(C)c2ccccc2-c2ccc3c4ccccc4n(-c4ccc(-c5nc(-c6ccc7c(c6)oc6ccccc67)nc(-n6c7ccccc7c7ccccc76)n5)cc4)c3c21] | 912 |
| 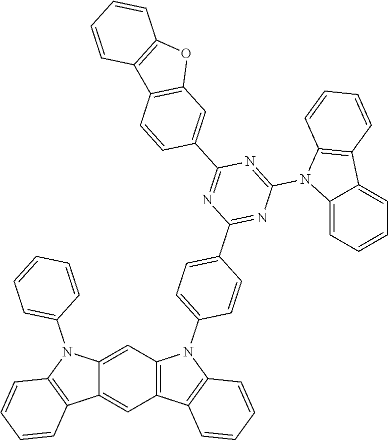 [SMILES: c1ccc(-n2c3ccccc3c3cc4c5ccccc5n(-c5ccc(-c6nc(-c7ccc8c(c7)oc7ccccc78)nc(-n7c8ccccc8c8ccccc87)n6)cc5)c4cc32)cc1] | 913 |
| 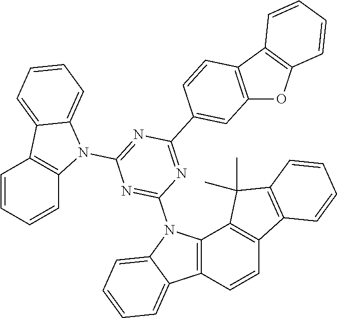 [SMILES: CC1(C)c2ccccc2-c2ccc3c4ccccc4n(-c4nc(-c5ccc6c(c5)oc5ccccc56)nc(-n5c6ccccc6c6ccccc65)n4)c3c21] | 914 |
| 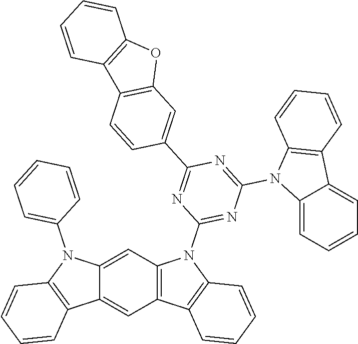 [SMILES: c1ccc(-n2c3ccccc3c3cc4c5ccccc5n(-c5nc(-c6ccc7c(c6)oc6ccccc67)nc(-n6c7ccccc7c7ccccc76)n5)c4cc32)cc1] | 915 |
| 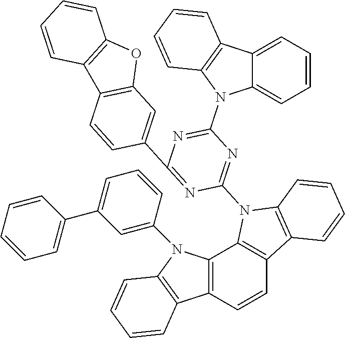 [SMILES: c1ccc(-c2cccc(-n3c4ccccc4c4ccc5c6ccccc6n(-c6nc(-c7ccc8c(c7)oc7ccccc78)nc(-n7c8ccccc8c8ccccc87)n6)c5c43)c2)cc1] | 916 |
| 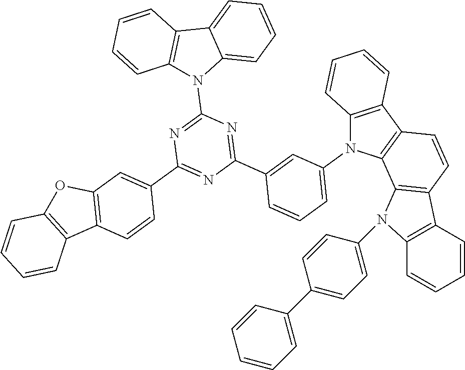 [SMILES: c1ccc(-c2ccc(-n3c4ccccc4c4ccc5c6ccccc6n(-c6cccc(-c7nc(-c8ccc9c(c8)oc8ccccc89)nc(-n8c9ccccc9c9ccccc98)n7)c6)c5c43)cc2)cc1] | 917 |
| 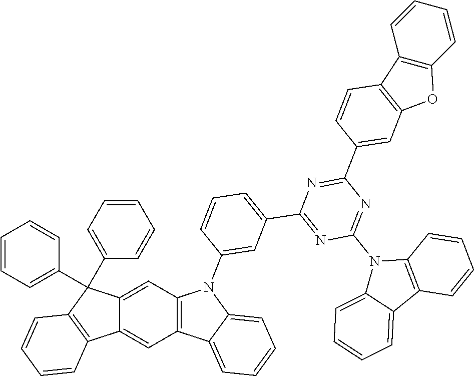 [SMILES: c1ccc(C2(c3ccccc3)c3ccccc3-c3cc4c5ccccc5n(-c5cccc(-c6nc(-c7ccc8c(c7)oc7ccccc78)nc(-n7c8ccccc8c8ccccc87)n6)c5)c4cc32)cc1] | 918 |
| 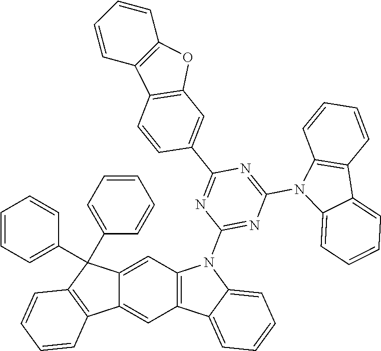 [SMILES: c1ccc(C2(c3ccccc3)c3ccccc3-c3cc4c5ccccc5n(-c5nc(-c6ccc7c(c6)oc6ccccc67)nc(-n6c7ccccc7c7ccccc76)n5)c4cc32)cc1] | 919 |
| 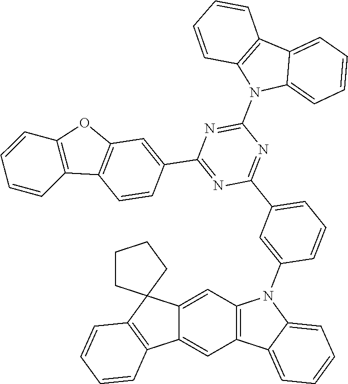 [SMILES: c1cc(-c2nc(-c3ccc4c(c3)oc3ccccc34)nc(-n3c4ccccc4c4ccccc43)n2)cc(-n2c3ccccc3c3cc4c(cc32)C2(CCCC2)c2ccccc2-4)c1] | 920 |
| 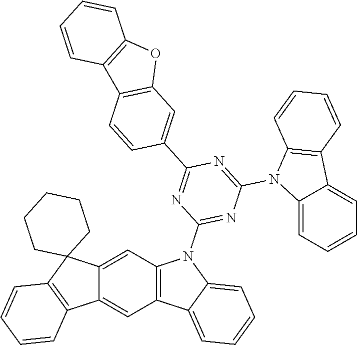 [SMILES: c1ccc2c(c1)-c1cc3c4ccccc4n(-c4nc(-c5ccc6c(c5)oc5ccccc56)nc(-n5c6ccccc6c6ccccc65)n4)c3cc1C21CCCCC1] | 921 |
| 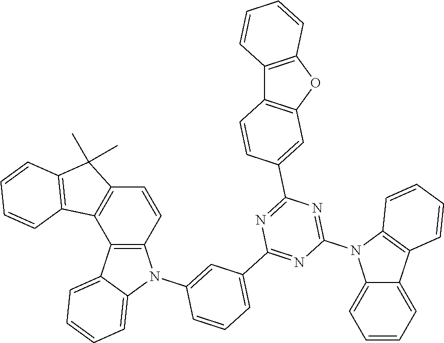 [SMILES: CC1(C)c2ccccc2-c2c1ccc1c2c2ccccc2n1-c1cccc(-c2nc(-c3ccc4c(c3)oc3ccccc34)nc(-n3c4ccccc4c4ccccc43)n2)c1] | 922 |
| 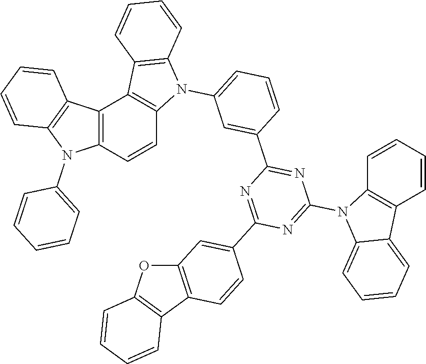 [SMILES: c1ccc(-n2c3ccccc3c3c4c5ccccc5n(-c5cccc(-c6nc(-c7ccc8c(c7)oc7ccccc78)nc(-n7c8ccccc8c8ccccc87)n6)c5)c4ccc32)cc1] | 923 |
| 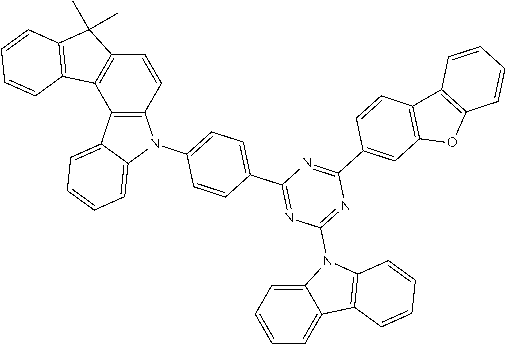 [SMILES: CC1(C)c2ccccc2-c2c1ccc1c2c2ccccc2n1-c1ccc(-c2nc(-c3ccc4c(c3)oc3ccccc34)nc(-n3c4ccccc4c4ccccc43)n2)cc1] | 924 |
| 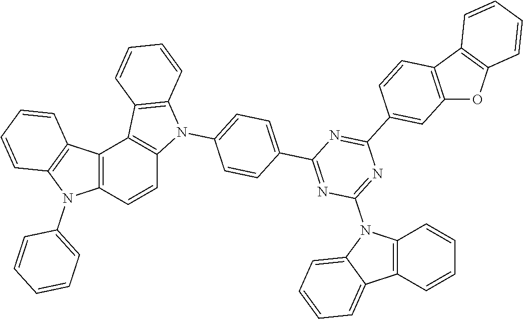 [SMILES: c1ccc(-n2c3ccccc3c3c4c5ccccc5n(-c5ccc(-c6nc(-c7ccc8c(c7)oc7ccccc78)nc(-n7c8ccccc8c8ccccc87)n6)cc5)c4ccc32)cc1] | 925 |
| 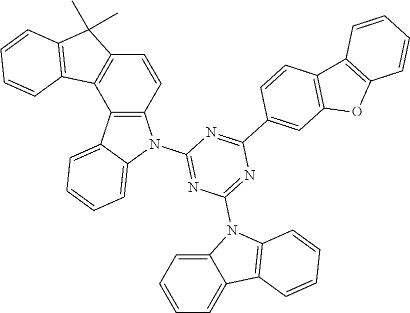 [SMILES: CC1(C)c2ccccc2-c2c1ccc1c2c2ccccc2n1-c1nc(-c2ccc3c(c2)oc2ccccc23)nc(-n2c3ccccc3c3ccccc32)n1] | 926 |
| 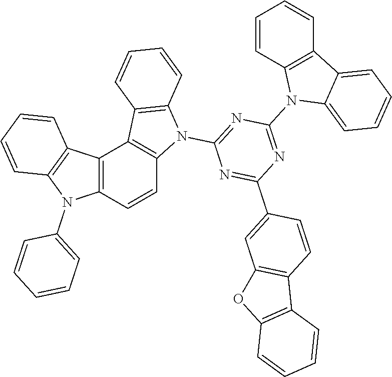 [SMILES: c1ccc(-n2c3ccccc3c3c4c5ccccc5n(-c5nc(-c6ccc7c(c6)oc6ccccc67)nc(-n6c7ccccc7c7ccccc76)n5)c4ccc32)cc1] | 927 |
| 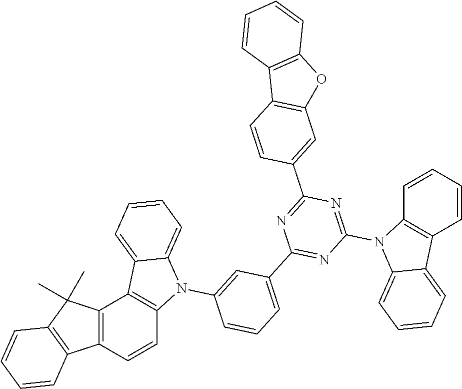 [SMILES: CC1(C)c2ccccc2-c2ccc3c(c21)c1ccccc1n3-c1cccc(-c2nc(-c3ccc4c(c3)oc3ccccc34)nc(-n3c4ccccc4c4ccccc43)n2)c1] | 928 |
| 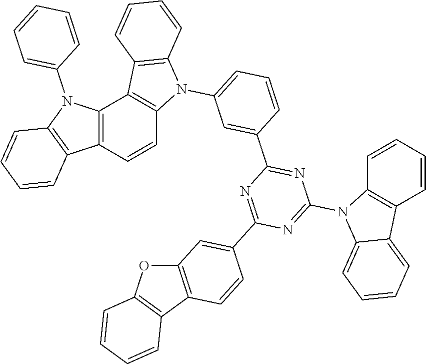 [SMILES: c1ccc(-n2c3ccccc3c3ccc4c(c5ccccc5n4-c4cccc(-c5nc(-c6ccc7c(c6)oc6ccccc67)nc(-n6c7ccccc7c7ccccc76)n5)c4)c32)cc1] | 929 |
| 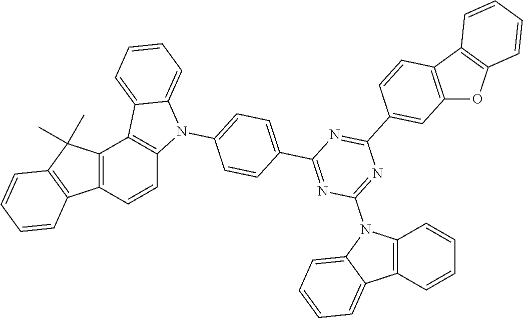 [SMILES: CC1(C)c2ccccc2-c2ccc3c(c21)c1ccccc1n3-c1ccc(-c2nc(-c3ccc4c(c3)oc3ccccc34)nc(-n3c4ccccc4c4ccccc43)n2)cc1] | 930 |
| 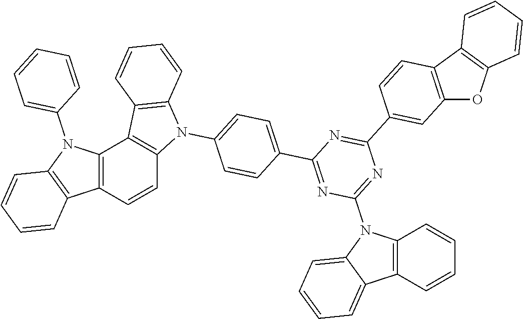 [SMILES: c1ccc(-n2c3ccccc3c3ccc4c(c5ccccc5n4-c4ccc(-c5nc(-c6ccc7c(c6)oc6ccccc67)nc(-n6c7ccccc7c7ccccc76)n5)cc4)c32)cc1] | 931 |
| 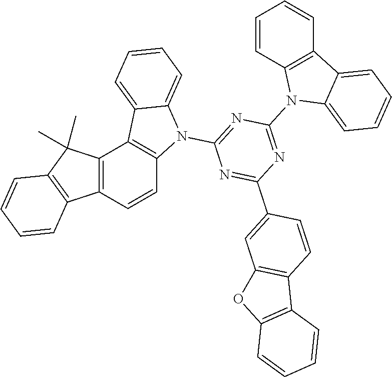 [SMILES: CC1(C)c2ccccc2-c2ccc3c(c21)c1ccccc1n3-c1nc(-c2ccc3c(c2)oc2ccccc23)nc(-n2c3ccccc3c3ccccc32)n1] | 932 |
| 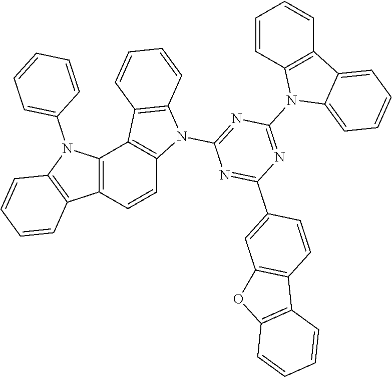 [SMILES: c1ccc(-n2c3ccccc3c3ccc4c(c5ccccc5n4-c4nc(-c5ccc6c(c5)oc5ccccc56)nc(-n5c6ccccc6c6ccccc65)n4)c32)cc1] | 933 |
| 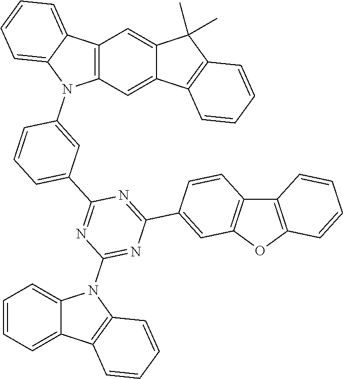 [SMILES: CC1(C)c2ccccc2-c2cc3c(cc21)c1ccccc1n3-c1cccc(-c2nc(-c3ccc4c(c3)oc3ccccc34)nc(-n3c4ccccc4c4ccccc43)n2)c1] | 934 |
| 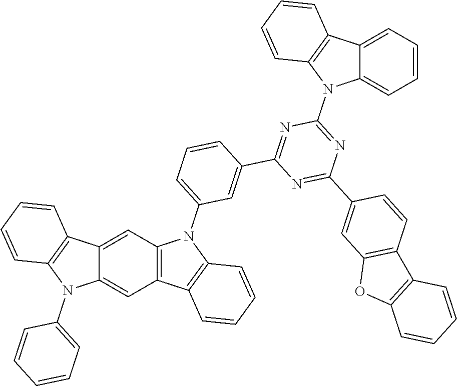 [SMILES: c1ccc(-n2c3ccccc3c3cc4c(cc32)c2ccccc2n4-c2cccc(-c3nc(-c4ccc5c(c4)oc4ccccc45)nc(-n4c5ccccc5c5ccccc54)n3)c2)cc1] | 935 |
| 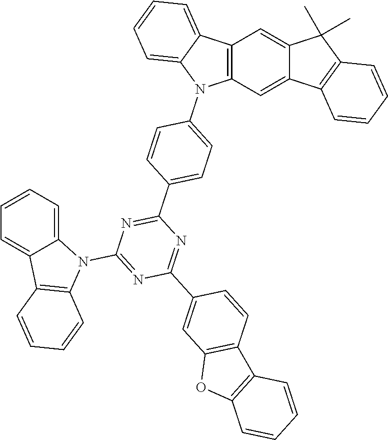 [SMILES: CC1(C)c2ccccc2-c2cc3c(cc21)c1ccccc1n3-c1ccc(-c2nc(-c3ccc4c(c3)oc3ccccc34)nc(-n3c4ccccc4c4ccccc43)n2)cc1] | 936 |
| 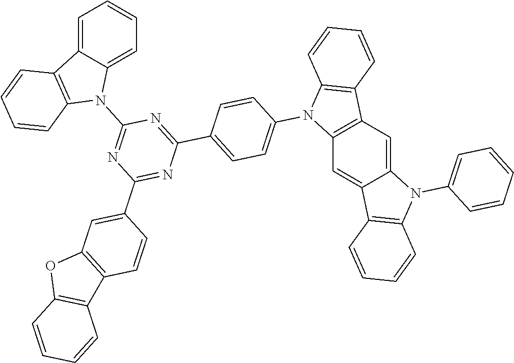 [SMILES: c1ccc(-n2c3ccccc3c3cc4c(cc32)c2ccccc2n4-c2ccc(-c3nc(-c4ccc5c(c4)oc4ccccc45)nc(-n4c5ccccc5c5ccccc54)n3)cc2)cc1] | 937 |
| 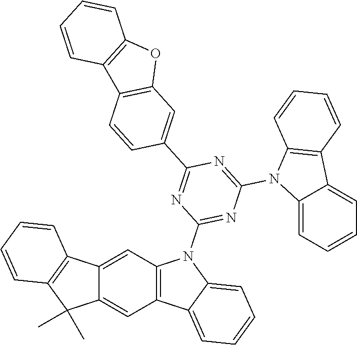 [SMILES: CC1(C)c2ccccc2-c2cc3c(cc21)c1ccccc1n3-c1nc(-c2ccc3c(c2)oc2ccccc23)nc(-n2c3ccccc3c3ccccc32)n1] | 938 |
| 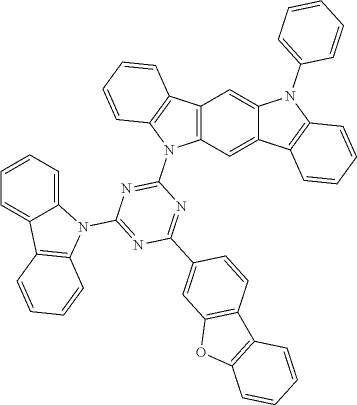 [SMILES: c1ccc(-n2c3ccccc3c3cc4c(cc32)c2ccccc2n4-c2nc(-c3ccc4c(c3)oc3ccccc34)nc(-n3c4ccccc4c4ccccc43)n2)cc1] | 939 |
| 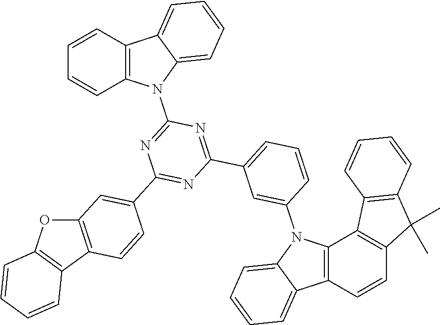 [SMILES: CC1(C)c2ccccc2-c2c1ccc1c3ccccc3n(-c3cccc(-c4nc(-c5ccc6c(c5)oc5ccccc56)nc(-n5c6ccccc6c6ccccc65)n4)c3)c21] | 940 |
| 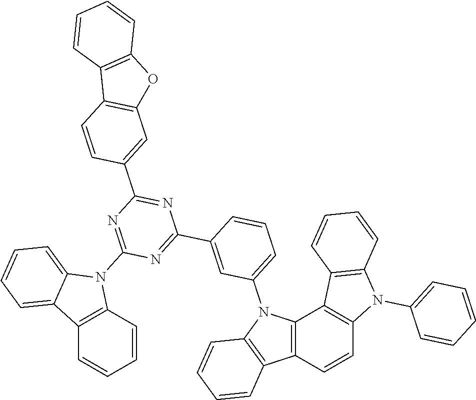 [SMILES: c1ccc(-n2c3ccccc3c3c2ccc2c4ccccc4n(-c4cccc(-c5nc(-c6ccc7c(c6)oc6ccccc67)nc(-n6c7ccccc7c7ccccc76)n5)c4)c23)cc1] | 941 |
| 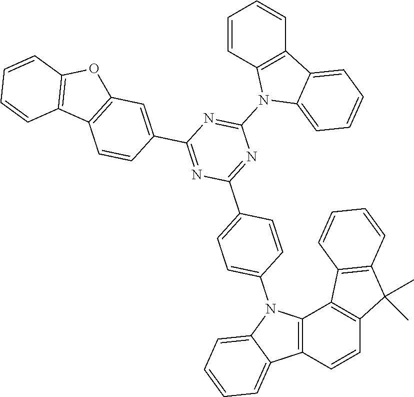 [SMILES: CC1(C)c2ccccc2-c2c1ccc1c3ccccc3n(-c3ccc(-c4nc(-c5ccc6c(c5)oc5ccccc56)nc(-n5c6ccccc6c6ccccc65)n4)cc3)c21] | 942 |
| 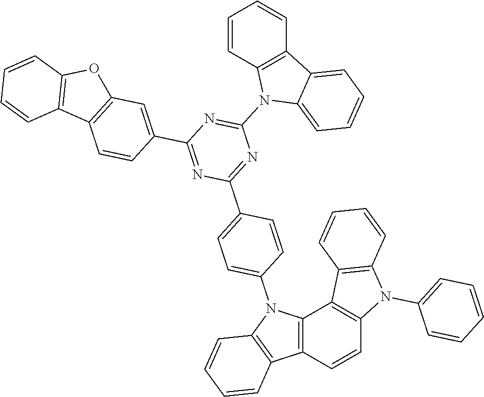 [SMILES: c1ccc(-n2c3ccccc3c3c2ccc2c4ccccc4n(-c4ccc(-c5nc(-c6ccc7c(c6)oc6ccccc67)nc(-n6c7ccccc7c7ccccc76)n5)cc4)c23)cc1] | 943 |
| 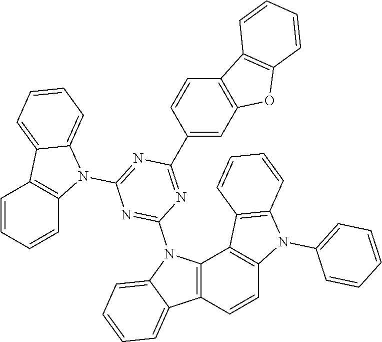 [SMILES: c1ccc(-n2c3ccccc3c3c2ccc2c4ccccc4n(-c4nc(-c5ccc6c(c5)oc5ccccc56)nc(-n5c6ccccc6c6ccccc65)n4)c23)cc1] | 944 |
| 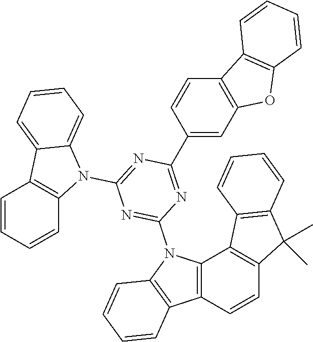 [SMILES: CC1(C)c2ccccc2-c2c1ccc1c3ccccc3n(-c3nc(-c4ccc5c(c4)oc4ccccc45)nc(-n4c5ccccc5c5ccccc54)n3)c21] | 945 |
| 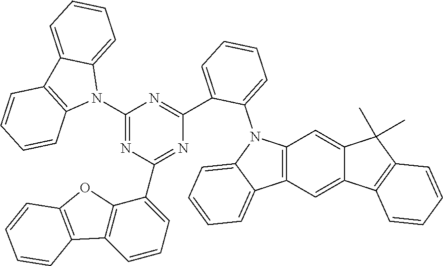 [SMILES: CC1(C)c2ccccc2-c2cc3c4ccccc4n(-c4ccccc4-c4nc(-c5cccc6c5oc5ccccc56)nc(-n5c6ccccc6c6ccccc65)n4)c3cc21] | 946 |
| 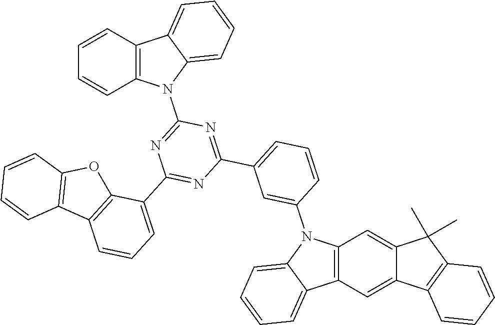 [SMILES: CC1(C)c2ccccc2-c2cc3c4ccccc4n(-c4cccc(-c5nc(-c6cccc7c6oc6ccccc67)nc(-n6c7ccccc7c7ccccc76)n5)c4)c3cc21] | 947 |
| 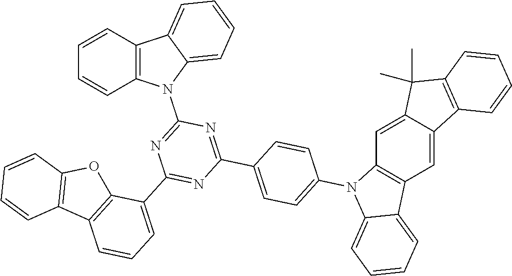 [SMILES: CC1(C)c2ccccc2-c2cc3c4ccccc4n(-c4ccc(-c5nc(-c6cccc7c6oc6ccccc67)nc(-n6c7ccccc7c7ccccc76)n5)cc4)c3cc21] | 948 |
| 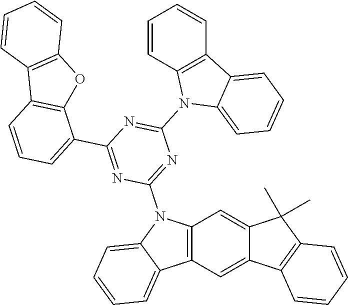 [SMILES: CC1(C)c2ccccc2-c2cc3c4ccccc4n(-c4nc(-c5cccc6c5oc5ccccc56)nc(-n5c6ccccc6c6ccccc65)n4)c3cc21] | 949 |
| 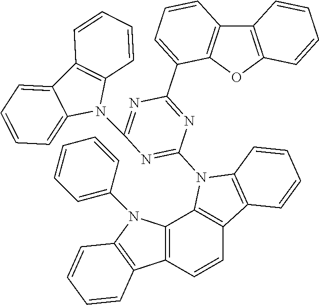 [SMILES: c1ccc(-n2c3ccccc3c3ccc4c5ccccc5n(-c5nc(-c6cccc7c6oc6ccccc67)nc(-n6c7ccccc7c7ccccc76)n5)c4c32)cc1] | 950 |
| 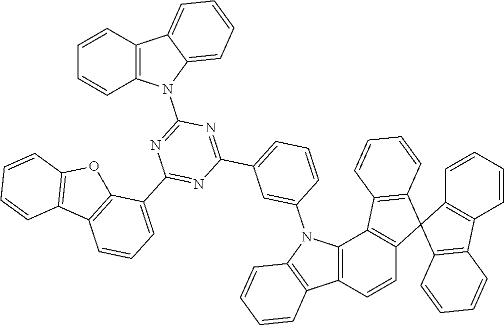 [SMILES: c1cc(-c2nc(-c3cccc4c3oc3ccccc34)nc(-n3c4ccccc4c4ccccc43)n2)cc(-n2c3ccccc3c3ccc4c(c32)-c2ccccc2C42c3ccccc3-c3ccccc32)c1] | 951 |
| 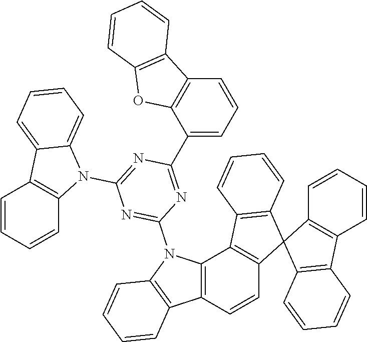 [SMILES: c1ccc2c(c1)-c1ccccc1C21c2ccccc2-c2c1ccc1c3ccccc3n(-c3nc(-c4cccc5c4oc4ccccc45)nc(-n4c5ccccc5c5ccccc54)n3)c21] | 952 |
| 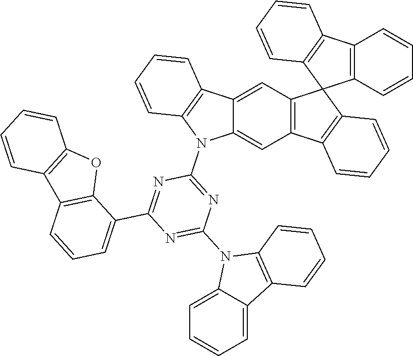 [SMILES: c1ccc2c(c1)-c1ccccc1C21c2ccccc2-c2cc3c(cc21)c1ccccc1n3-c1nc(-c2cccc3c2oc2ccccc23)nc(-n2c3ccccc3c3ccccc32)n1] | 953 |
| 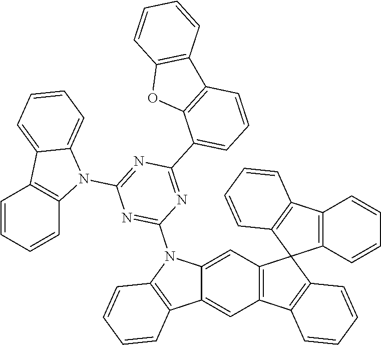 [SMILES: c1ccc2c(c1)-c1ccccc1C21c2ccccc2-c2cc3c4ccccc4n(-c4nc(-c5cccc6c5oc5ccccc56)nc(-n5c6ccccc6c6ccccc65)n4)c3cc21] | 954 |
| 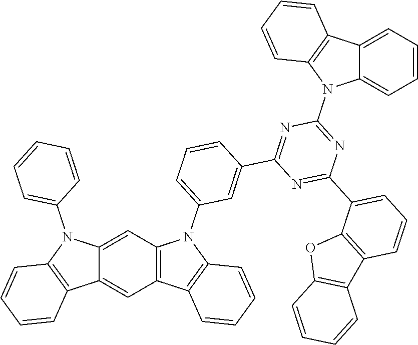 [SMILES: c1ccc(-n2c3ccccc3c3cc4c5ccccc5n(-c5cccc(-c6nc(-c7cccc8c7oc7ccccc78)nc(-n7c8ccccc8c8ccccc87)n6)c5)c4cc32)cc1] | 955 |
| 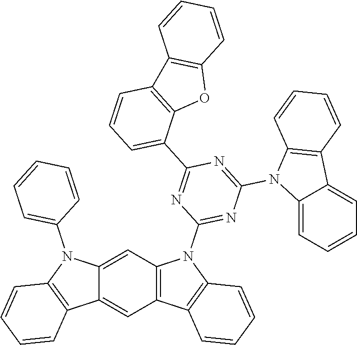 [SMILES: c1ccc(-n2c3ccccc3c3cc4c5ccccc5n(-c5nc(-c6cccc7c6oc6ccccc67)nc(-n6c7ccccc7c7ccccc76)n5)c4cc32)cc1] | 956 |
| 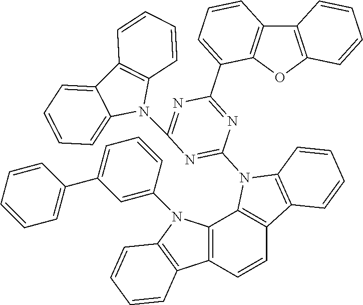 [SMILES: c1ccc(-c2cccc(-n3c4ccccc4c4ccc5c6ccccc6n(-c6nc(-c7cccc8c7oc7ccccc78)nc(-n7c8ccccc8c8ccccc87)n6)c5c43)c2)cc1] | 957 |
| 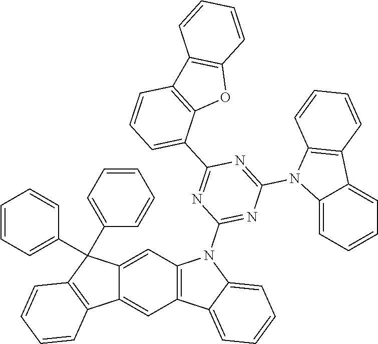 [SMILES: c1ccc(C2(c3ccccc3)c3ccccc3-c3cc4c5ccccc5n(-c5nc(-c6cccc7c6oc6ccccc67)nc(-n6c7ccccc7c7ccccc76)n5)c4cc32)cc1] | 958 |
| 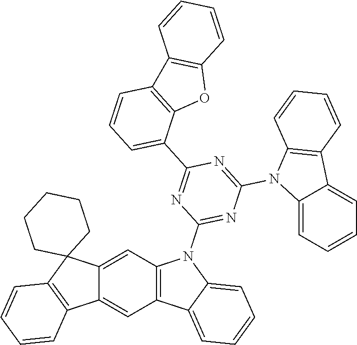 [SMILES: c1ccc2c(c1)-c1cc3c4ccccc4n(-c4nc(-c5cccc6c5oc5ccccc56)nc(-n5c6ccccc6c6ccccc65)n4)c3cc1C21CCCCC1] | 959 |
| 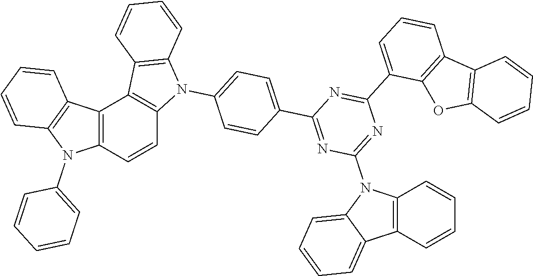 [SMILES: c1ccc(-n2c3ccccc3c3c4c5ccccc5n(-c5ccc(-c6nc(-c7cccc8c7oc7ccccc78)nc(-n7c8ccccc8c8ccccc87)n6)cc5)c4ccc32)cc1] | 960 |
| 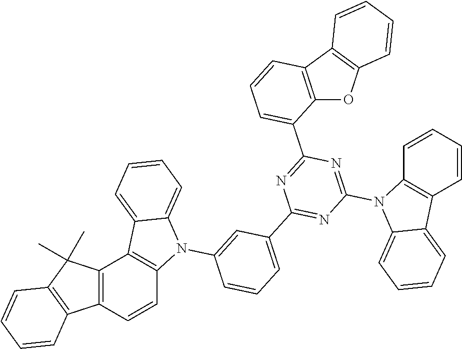 [SMILES: CC1(C)c2ccccc2-c2ccc3c(c21)c1ccccc1n3-c1cccc(-c2nc(-c3cccc4c3oc3ccccc34)nc(-n3c4ccccc4c4ccccc43)n2)c1] | 961 |
| 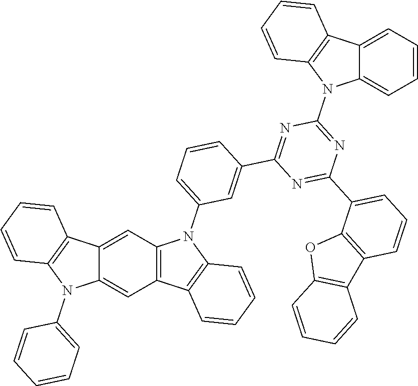 [SMILES: c1ccc(-n2c3ccccc3c3cc4c(cc32)c2ccccc2n4-c2cccc(-c3nc(-c4cccc5c4oc4ccccc45)nc(-n4c5ccccc5c5ccccc54)n3)c2)cc1] | 962 |
| 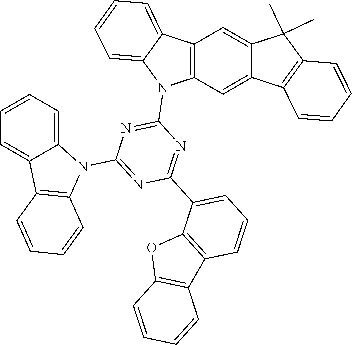 [SMILES: CC1(C)c2ccccc2-c2cc3c(cc21)c1ccccc1n3-c1nc(-c2cccc3c2oc2ccccc23)nc(-n2c3ccccc3c3ccccc32)n1] | 963 |
| 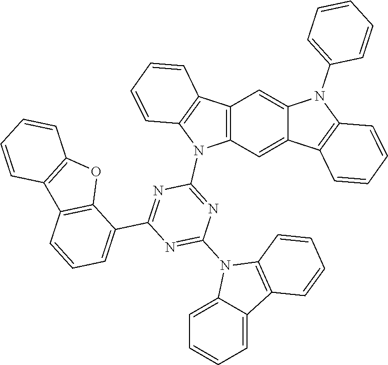 [SMILES: c1ccc(-n2c3ccccc3c3cc4c(cc32)c2ccccc2n4-c2nc(-c3cccc4c3oc3ccccc34)nc(-n3c4ccccc4c4ccccc43)n2)cc1] | 964 |
| 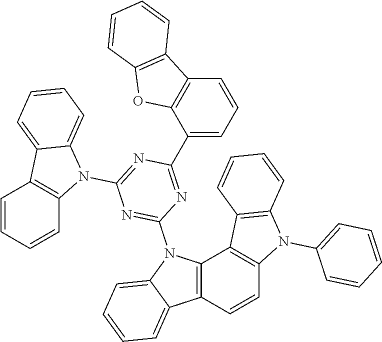 [SMILES: c1ccc(-n2c3ccccc3c3c2ccc2c4ccccc4n(-c4nc(-c5cccc6c5oc5ccccc56)nc(-n5c6ccccc6c6ccccc65)n4)c23)cc1] | 965 |
| 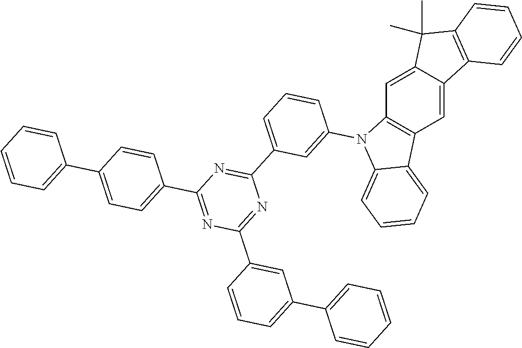 [SMILES: CC1(C)c2ccccc2-c2cc3c4ccccc4n(-c4cccc(-c5nc(-c6ccc(-c7ccccc7)cc6)nc(-c6cccc(-c7ccccc7)c6)n5)c4)c3cc21] | 966 |
| 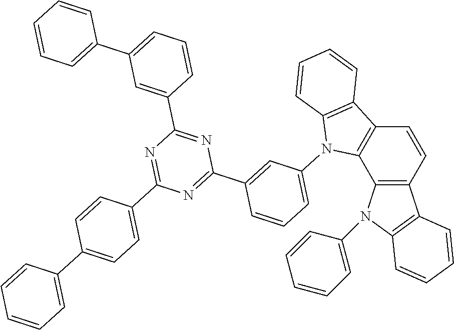 [SMILES: c1ccc(-c2ccc(-c3nc(-c4cccc(-c5ccccc5)c4)nc(-c4cccc(-n5c6ccccc6c6ccc7c8ccccc8n(-c8ccccc8)c7c65)c4)n3)cc2)cc1] | 967 |
| 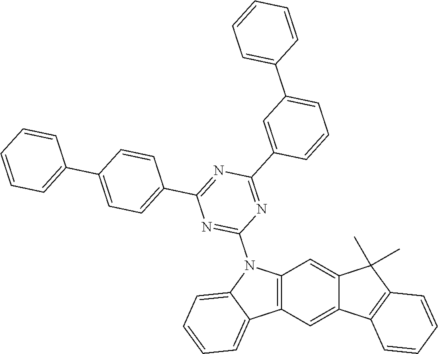 [SMILES: CC1(C)c2ccccc2-c2cc3c4ccccc4n(-c4nc(-c5ccc(-c6ccccc6)cc5)nc(-c5cccc(-c6ccccc6)c5)n4)c3cc21] | 968 |
| 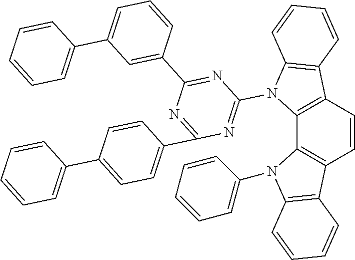 [SMILES: c1ccc(-c2ccc(-c3nc(-c4cccc(-c5ccccc5)c4)nc(-n4c5ccccc5c5ccc6c7ccccc7n(-c7ccccc7)c6c54)n3)cc2)cc1] | 969 |
| 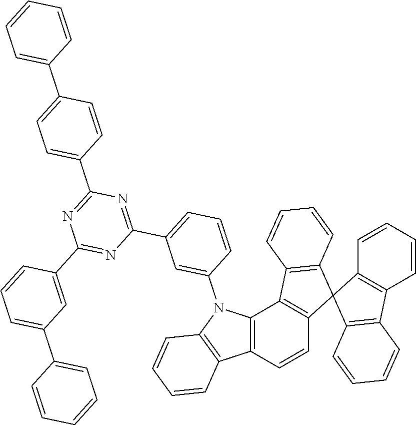 [SMILES: c1ccc(-c2ccc(-c3nc(-c4cccc(-c5ccccc5)c4)nc(-c4cccc(-n5c6ccccc6c6ccc7c(c65)-c5ccccc5C75c6ccccc6-c6ccccc65)c4)n3)cc2)cc1] | 970 |
| 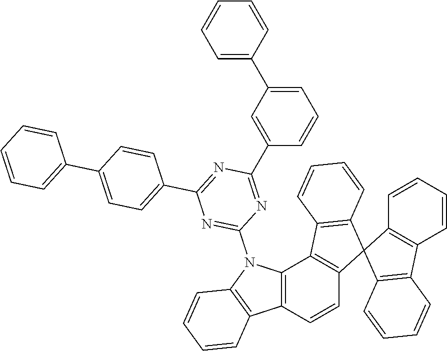 [SMILES: c1ccc(-c2ccc(-c3nc(-c4cccc(-c5ccccc5)c4)nc(-n4c5ccccc5c5ccc6c(c54)-c4ccccc4C64c5ccccc5-c5ccccc54)n3)cc2)cc1] | 971 |
| 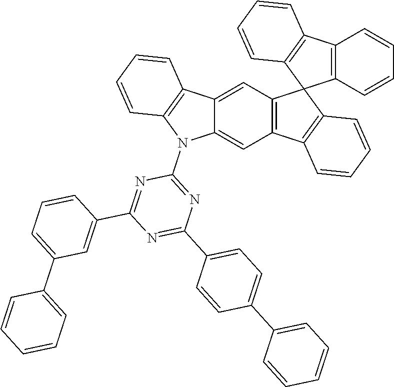 [SMILES: c1ccc(-c2ccc(-c3nc(-c4cccc(-c5ccccc5)c4)nc(-n4c5ccccc5c5cc6c(cc54)-c4ccccc4C64c5ccccc5-c5ccccc54)n3)cc2)cc1] | 972 |
| 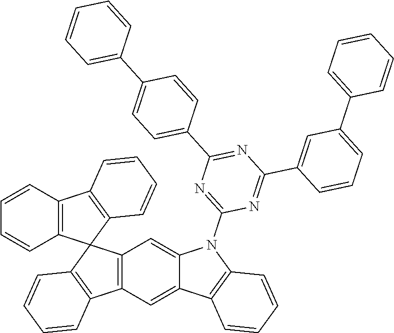 [SMILES: c1ccc(-c2ccc(-c3nc(-c4cccc(-c5ccccc5)c4)nc(-n4c5ccccc5c5cc6c(cc54)C4(c5ccccc5-c5ccccc54)c4ccccc4-6)n3)cc2)cc1] | 973 |
| 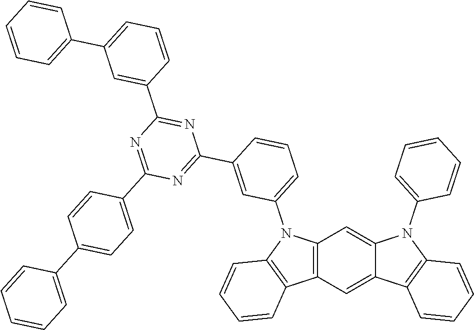 [SMILES: c1ccc(-c2ccc(-c3nc(-c4cccc(-c5ccccc5)c4)nc(-c4cccc(-n5c6ccccc6c6cc7c8ccccc8n(-c8ccccc8)c7cc65)c4)n3)cc2)cc1] | 974 |
| 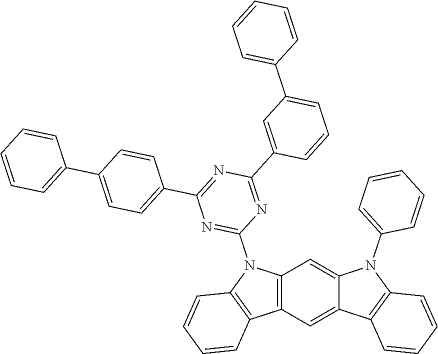 [SMILES: c1ccc(-c2ccc(-c3nc(-c4cccc(-c5ccccc5)c4)nc(-n4c5ccccc5c5cc6c7ccccc7n(-c7ccccc7)c6cc54)n3)cc2)cc1] | 975 |
| 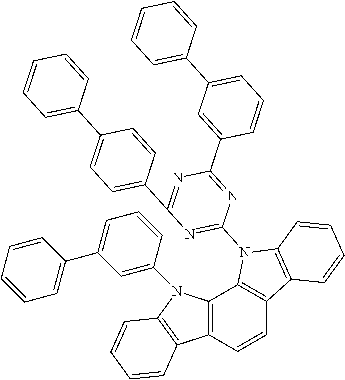 [SMILES: c1ccc(-c2ccc(-c3nc(-c4cccc(-c5ccccc5)c4)nc(-n4c5ccccc5c5ccc6c7ccccc7n(-c7cccc(-c8ccccc8)c7)c6c54)n3)cc2)cc1] | 976 |
| 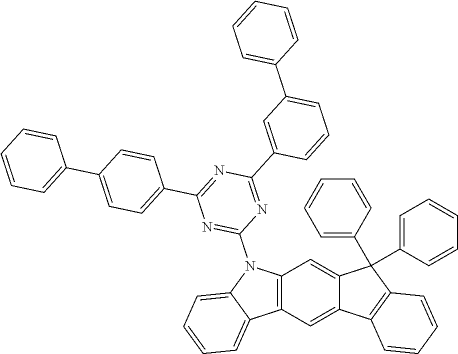 [SMILES: c1ccc(-c2ccc(-c3nc(-c4cccc(-c5ccccc5)c4)nc(-n4c5ccccc5c5cc6c(cc54)C(c4ccccc4)(c4ccccc4)c4ccccc4-6)n3)cc2)cc1] | 977 |
| 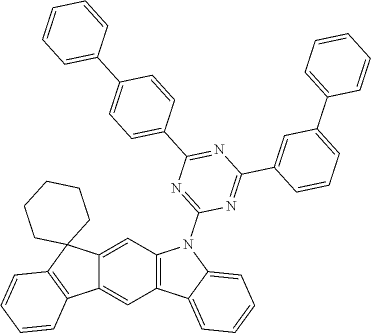 [SMILES: c1ccc(-c2ccc(-c3nc(-c4cccc(-c5ccccc5)c4)nc(-n4c5ccccc5c5cc6c(cc54)C4(CCCCC4)c4ccccc4-6)n3)cc2)cc1] | 978 |
| 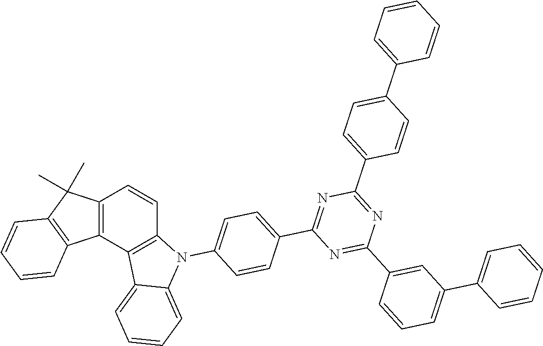 [SMILES: CC1(C)c2ccccc2-c2c1ccc1c2c2ccccc2n1-c1ccc(-c2nc(-c3ccc(-c4ccccc4)cc3)nc(-c3cccc(-c4ccccc4)c3)n2)cc1] | 979 |
| 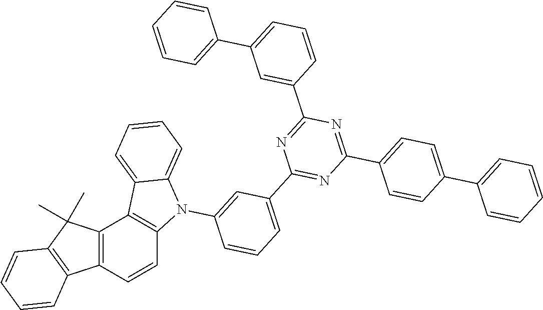 [SMILES: CC1(C)c2ccccc2-c2ccc3c(c21)c1ccccc1n3-c1cccc(-c2nc(-c3ccc(-c4ccccc4)cc3)nc(-c3cccc(-c4ccccc4)c3)n2)c1] | 980 |
| 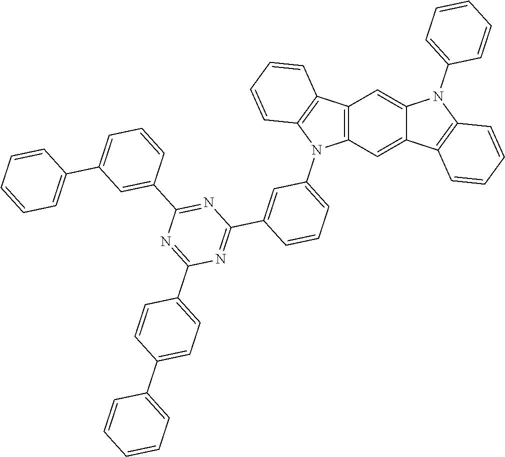 [SMILES: c1ccc(-c2ccc(-c3nc(-c4cccc(-c5ccccc5)c4)nc(-c4cccc(-n5c6ccccc6c6cc7c(cc65)c5ccccc5n7-c5ccccc5)c4)n3)cc2)cc1] | 981 |
| 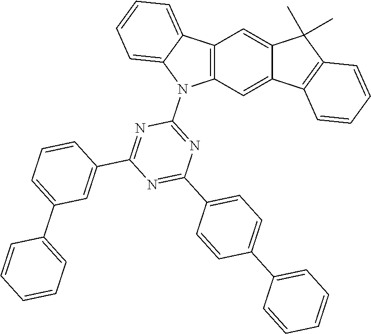 [SMILES: CC1(C)c2ccccc2-c2cc3c(cc21)c1ccccc1n3-c1nc(-c2ccc(-c3ccccc3)cc2)nc(-c2cccc(-c3ccccc3)c2)n1] | 982 |
| 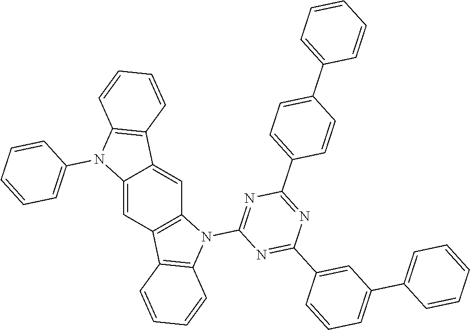 [SMILES: c1ccc(-c2ccc(-c3nc(-c4cccc(-c5ccccc5)c4)nc(-n4c5ccccc5c5cc6c(cc54)c4ccccc4n6-c4ccccc4)n3)cc2)cc1] | 983 |
| 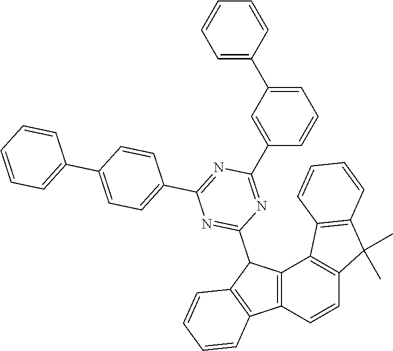 [SMILES: CC1(C)c2ccccc2-c2c1ccc1c2C(c2nc(-c3ccc(-c4ccccc4)cc3)nc(-c3cccc(-c4ccccc4)c3)n2)c2ccccc2-1] | 984 |
| 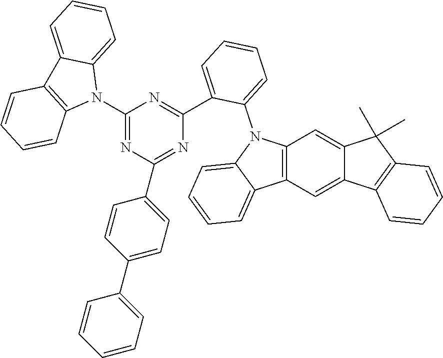 [SMILES: CC1(C)c2ccccc2-c2cc3c4ccccc4n(-c4ccccc4-c4nc(-c5ccc(-c6ccccc6)cc5)nc(-n5c6ccccc6c6ccccc65)n4)c3cc21] | 985 |
| 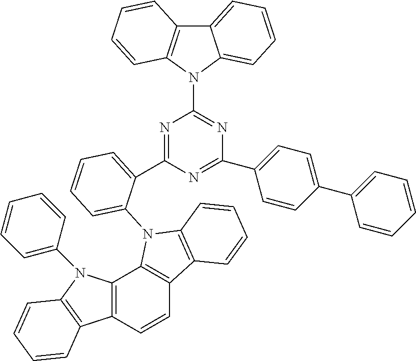 [SMILES: c1ccc(-c2ccc(-c3nc(-c4ccccc4-n4c5ccccc5c5ccc6c7ccccc7n(-c7ccccc7)c6c54)nc(-n4c5ccccc5c5ccccc54)n3)cc2)cc1] | 986 |
| 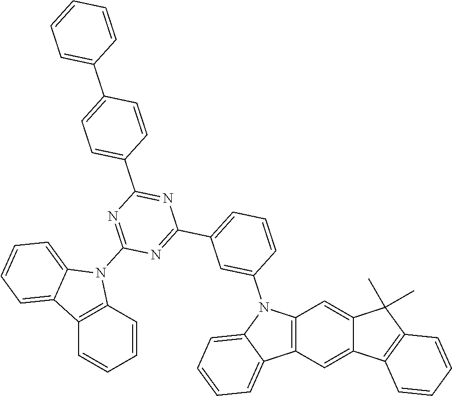 [SMILES: CC1(C)c2ccccc2-c2cc3c4ccccc4n(-c4cccc(-c5nc(-c6ccc(-c7ccccc7)cc6)nc(-n6c7ccccc7c7ccccc76)n5)c4)c3cc21] | 987 |
| 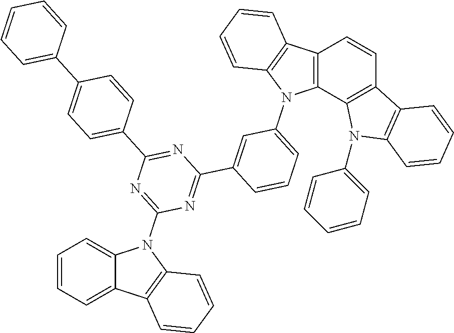 [SMILES: c1ccc(-c2ccc(-c3nc(-c4cccc(-n5c6ccccc6c6ccc7c8ccccc8n(-c8ccccc8)c7c65)c4)nc(-n4c5ccccc5c5ccccc54)n3)cc2)cc1] | 988 |
| 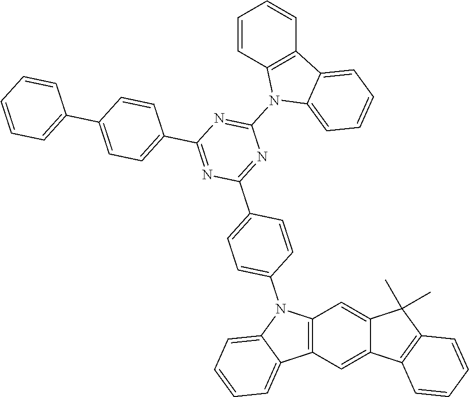 [SMILES: CC1(C)c2ccccc2-c2cc3c4ccccc4n(-c4ccc(-c5nc(-c6ccc(-c7ccccc7)cc6)nc(-n6c7ccccc7c7ccccc76)n5)cc4)c3cc21] | 989 |
| 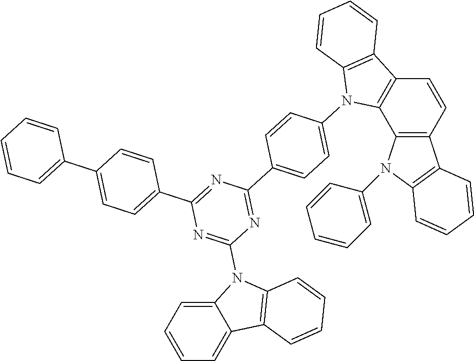 [SMILES: c1ccc(-c2ccc(-c3nc(-c4ccc(-n5c6ccccc6c6ccc7c8ccccc8n(-c8ccccc8)c7c65)cc4)nc(-n4c5ccccc5c5ccccc54)n3)cc2)cc1] | 990 |
| 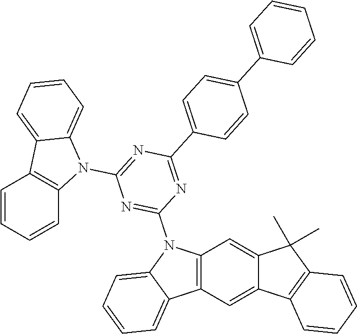 [SMILES: CC1(C)c2ccccc2-c2cc3c4ccccc4n(-c4nc(-c5ccc(-c6ccccc6)cc5)nc(-n5c6ccccc6c6ccccc65)n4)c3cc21] | 991 |
| 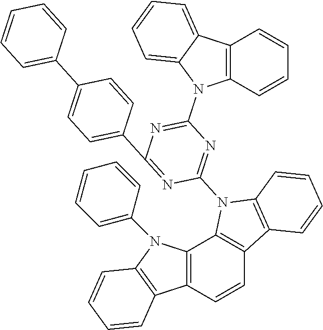 [SMILES: c1ccc(-c2ccc(-c3nc(-n4c5ccccc5c5ccccc54)nc(-n4c5ccccc5c5ccc6c7ccccc7n(-c7ccccc7)c6c54)n3)cc2)cc1] | 992 |
| 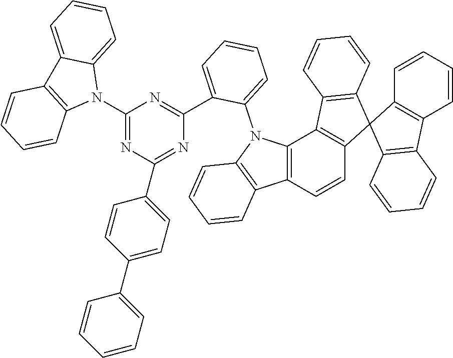 [SMILES: c1ccc(-c2ccc(-c3nc(-c4ccccc4-n4c5ccccc5c5ccc6c(c54)-c4ccccc4C64c5ccccc5-c5ccccc54)nc(-n4c5ccccc5c5ccccc54)n3)cc2)cc1] | 993 |
| 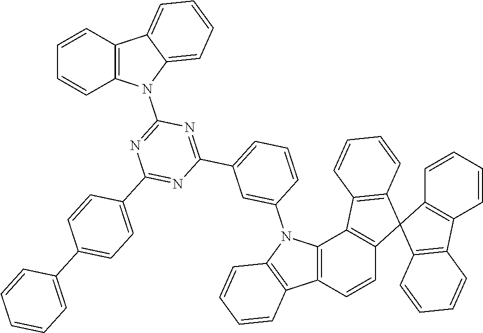 [SMILES: c1ccc(-c2ccc(-c3nc(-c4cccc(-n5c6ccccc6c6ccc7c(c65)-c5ccccc5C75c6ccccc6-c6ccccc65)c4)nc(-n4c5ccccc5c5ccccc54)n3)cc2)cc1] | 994 |
| 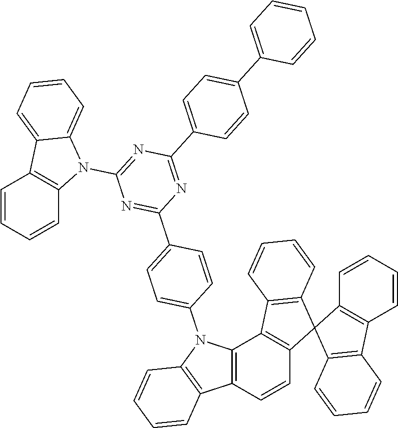 [SMILES: c1ccc(-c2ccc(-c3nc(-c4ccc(-n5c6ccccc6c6ccc7c(c65)-c5ccccc5C75c6ccccc6-c6ccccc65)cc4)nc(-n4c5ccccc5c5ccccc54)n3)cc2)cc1] | 995 |
| 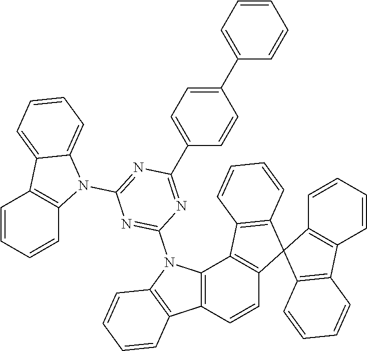 [SMILES: c1ccc(-c2ccc(-c3nc(-n4c5ccccc5c5ccccc54)nc(-n4c5ccccc5c5ccc6c(c54)-c4ccccc4C64c5ccccc5-c5ccccc54)n3)cc2)cc1] | 996 |
| 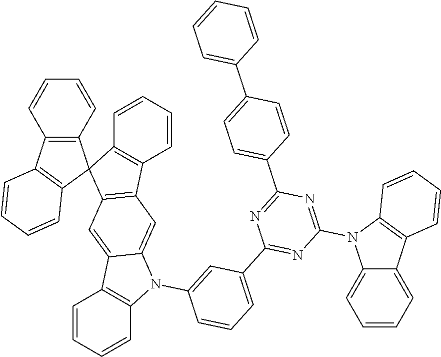 [SMILES: c1ccc(-c2ccc(-c3nc(-c4cccc(-n5c6ccccc6c6cc7c(cc65)-c5ccccc5C75c6ccccc6-c6ccccc65)c4)nc(-n4c5ccccc5c5ccccc54)n3)cc2)cc1] | 997 |
| 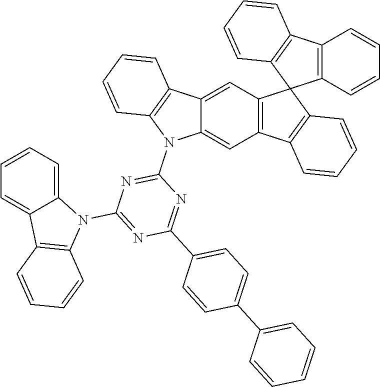 [SMILES: c1ccc(-c2ccc(-c3nc(-n4c5ccccc5c5ccccc54)nc(-n4c5ccccc5c5cc6c(cc54)-c4ccccc4C64c5ccccc5-c5ccccc54)n3)cc2)cc1] | 998 |
| 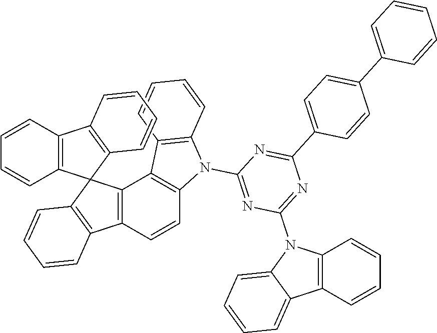 [SMILES: c1ccc(-c2ccc(-c3nc(-n4c5ccccc5c5ccccc54)nc(-n4c5ccccc5c5c6c(ccc54)-c4ccccc4C64c5ccccc5-c5ccccc54)n3)cc2)cc1] | 999 |
| 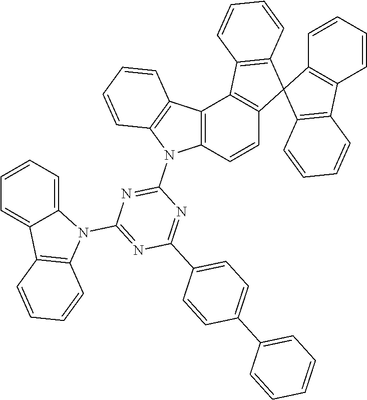 [SMILES: c1ccc(-c2ccc(-c3nc(-n4c5ccccc5c5ccccc54)nc(-n4c5ccccc5c5c6c(ccc54)C4(c5ccccc5-c5ccccc54)c4ccccc4-6)n3)cc2)cc1] | 1000 |
| 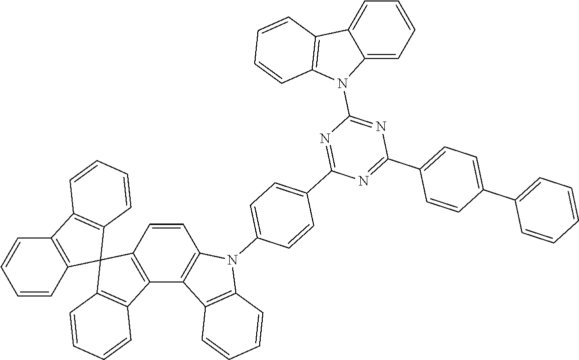 [SMILES: c1ccc(-c2ccc(-c3nc(-c4ccc(-n5c6ccccc6c6c7c(ccc65)C5(c6ccccc6-c6ccccc65)c5ccccc5-7)cc4)nc(-n4c5ccccc5c5ccccc54)n3)cc2)cc1] | 1001 |
| 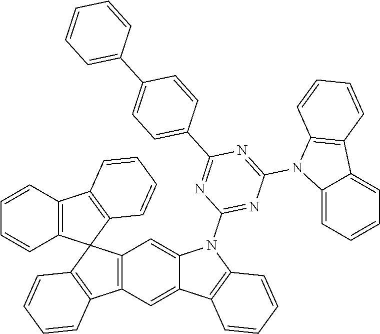 [SMILES: c1ccc(-c2ccc(-c3nc(-n4c5ccccc5c5ccccc54)nc(-n4c5ccccc5c5cc6c(cc54)C4(c5ccccc5-c5ccccc54)c4ccccc4-6)n3)cc2)cc1] | 1002 |
| 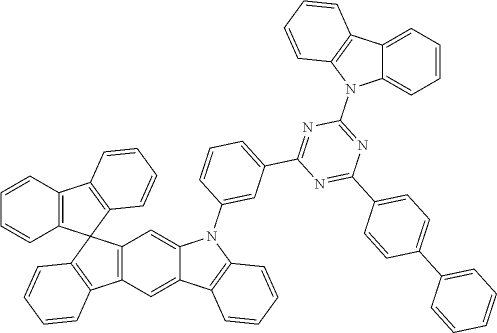 [SMILES: c1ccc(-c2ccc(-c3nc(-c4cccc(-n5c6ccccc6c6cc7c(cc65)C5(c6ccccc6-c6ccccc65)c5ccccc5-7)c4)nc(-n4c5ccccc5c5ccccc54)n3)cc2)cc1] | 1003 |
| 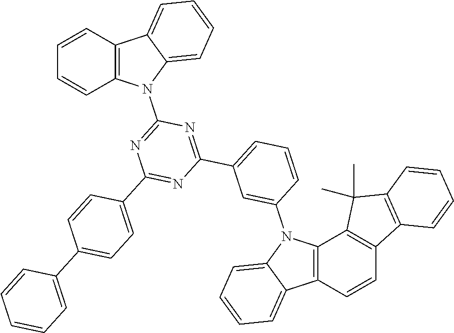 [SMILES: CC1(C)c2ccccc2-c2ccc3c4ccccc4n(-c4cccc(-c5nc(-c6ccc(-c7ccccc7)cc6)nc(-n6c7ccccc7c7ccccc76)n5)c4)c3c21] | 1004 |
| 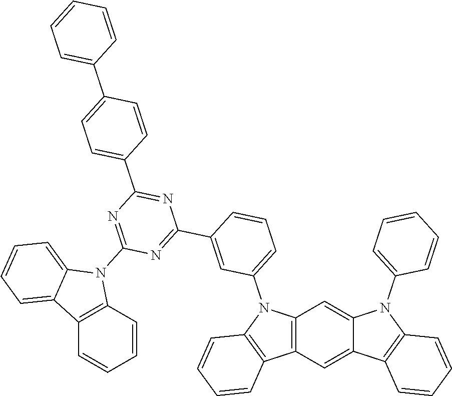 [SMILES: c1ccc(-c2ccc(-c3nc(-c4cccc(-n5c6ccccc6c6cc7c8ccccc8n(-c8ccccc8)c7cc65)c4)nc(-n4c5ccccc5c5ccccc54)n3)cc2)cc1] | 1005 |
| 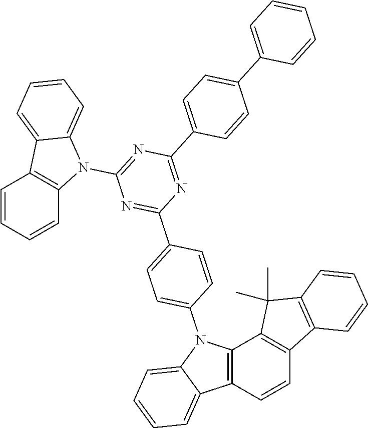 [SMILES: CC1(C)c2ccccc2-c2ccc3c4ccccc4n(-c4ccc(-c5nc(-c6ccc(-c7ccccc7)cc6)nc(-n6c7ccccc7c7ccccc76)n5)cc4)c3c21] | 1006 |
| 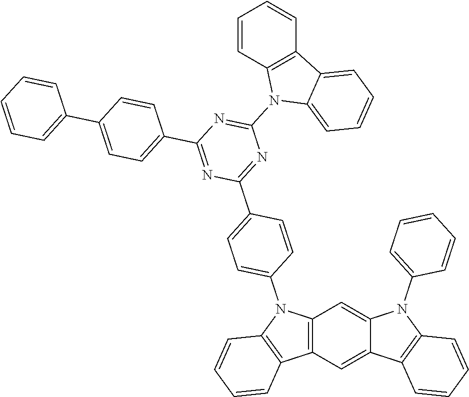 [SMILES: c1ccc(-c2ccc(-c3nc(-c4ccc(-n5c6ccccc6c6cc7c8ccccc8n(-c8ccccc8)c7cc65)cc4)nc(-n4c5ccccc5c5ccccc54)n3)cc2)cc1] | 1007 |
| 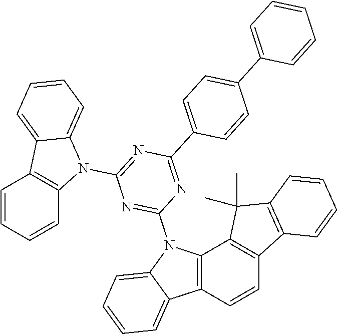 [SMILES: CC1(C)c2ccccc2-c2ccc3c4ccccc4n(-c4nc(-c5ccc(-c6ccccc6)cc5)nc(-n5c6ccccc6c6ccccc65)n4)c3c21] | 1008 |
| 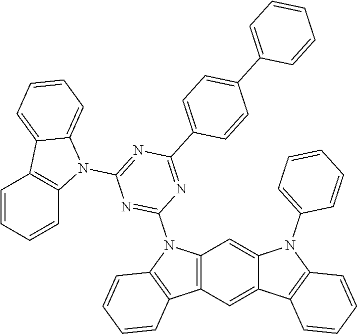 [SMILES: c1ccc(-c2ccc(-c3nc(-n4c5ccccc5c5ccccc54)nc(-n4c5ccccc5c5cc6c7ccccc7n(-c7ccccc7)c6cc54)n3)cc2)cc1] | 1009 |
| 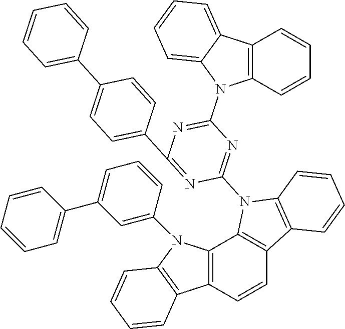 [SMILES: c1ccc(-c2ccc(-c3nc(-n4c5ccccc5c5ccccc54)nc(-n4c5ccccc5c5ccc6c7ccccc7n(-c7cccc(-c8ccccc8)c7)c6c54)n3)cc2)cc1] | 1010 |
| 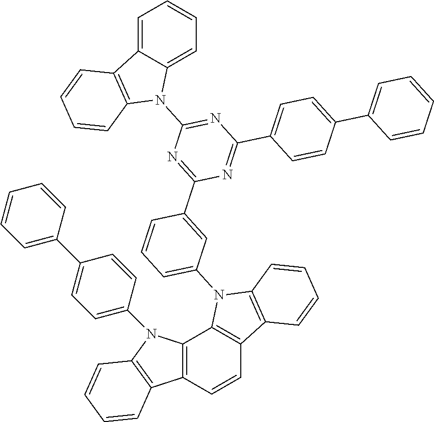 [SMILES: c1ccc(-c2ccc(-c3nc(-c4cccc(-n5c6ccccc6c6ccc7c8ccccc8n(-c8ccc(-c9ccccc9)cc8)c7c65)c4)nc(-n4c5ccccc5c5ccccc54)n3)cc2)cc1] | 1011 |
| 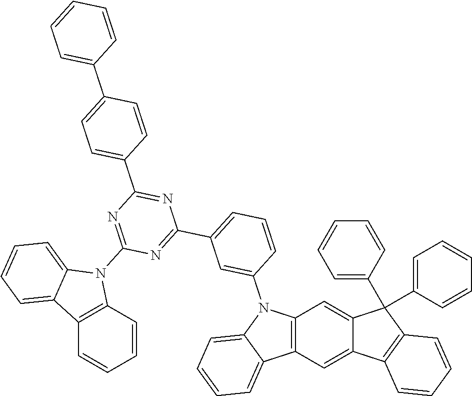 [SMILES: c1ccc(-c2ccc(-c3nc(-c4cccc(-n5c6ccccc6c6cc7c(cc65)C(c5ccccc5)(c5ccccc5)c5ccccc5-7)c4)nc(-n4c5ccccc5c5ccccc54)n3)cc2)cc1] | 1012 |
| 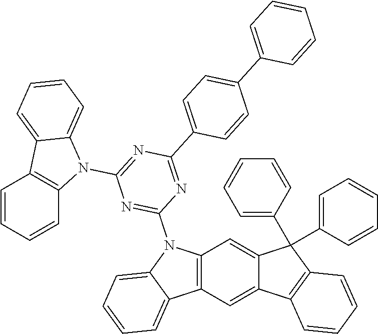 [SMILES: c1ccc(-c2ccc(-c3nc(-n4c5ccccc5c5ccccc54)nc(-n4c5ccccc5c5cc6c(cc54)C(c4ccccc4)(c4ccccc4)c4ccccc4-6)n3)cc2)cc1] | 1013 |
| 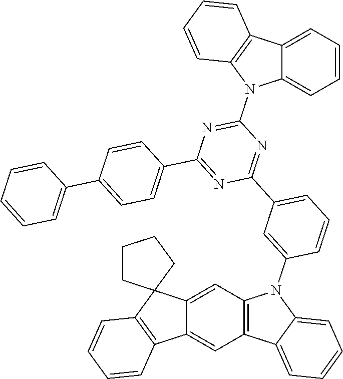 [SMILES: c1ccc(-c2ccc(-c3nc(-c4cccc(-n5c6ccccc6c6cc7c(cc65)C5(CCCC5)c5ccccc5-7)c4)nc(-n4c5ccccc5c5ccccc54)n3)cc2)cc1] | 1014 |
| 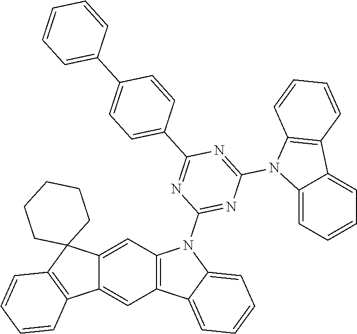 [SMILES: c1ccc(-c2ccc(-c3nc(-n4c5ccccc5c5ccccc54)nc(-n4c5ccccc5c5cc6c(cc54)C4(CCCCC4)c4ccccc4-6)n3)cc2)cc1] | 1015 |
| 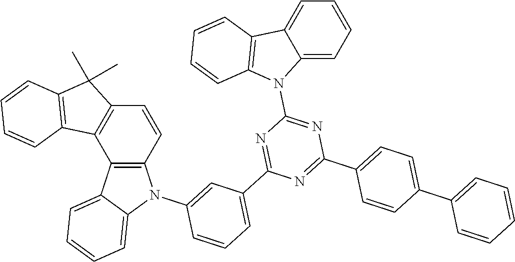 [SMILES: CC1(C)c2ccccc2-c2c1ccc1c2c2ccccc2n1-c1cccc(-c2nc(-c3ccc(-c4ccccc4)cc3)nc(-n3c4ccccc4c4ccccc43)n2)c1] | 1016 |
| 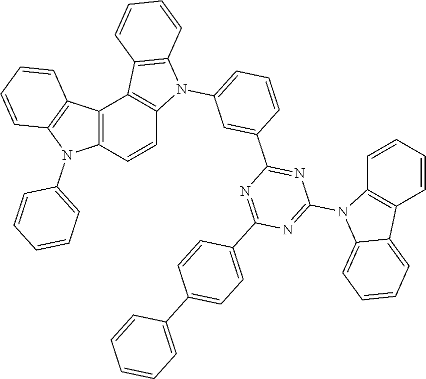 [SMILES: c1ccc(-c2ccc(-c3nc(-c4cccc(-n5c6ccccc6c6c7c8ccccc8n(-c8ccccc8)c7ccc65)c4)nc(-n4c5ccccc5c5ccccc54)n3)cc2)cc1] | 1017 |
| 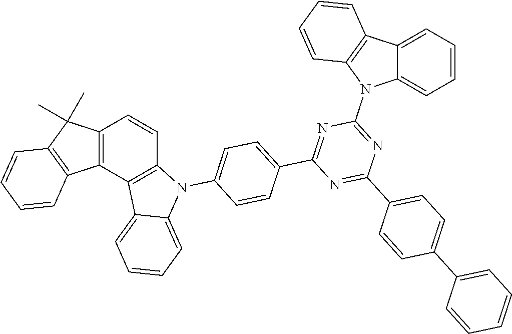 [SMILES: CC1(C)c2ccccc2-c2c1ccc1c2c2ccccc2n1-c1ccc(-c2nc(-c3ccc(-c4ccccc4)cc3)nc(-n3c4ccccc4c4ccccc43)n2)cc1] | 1018 |
| 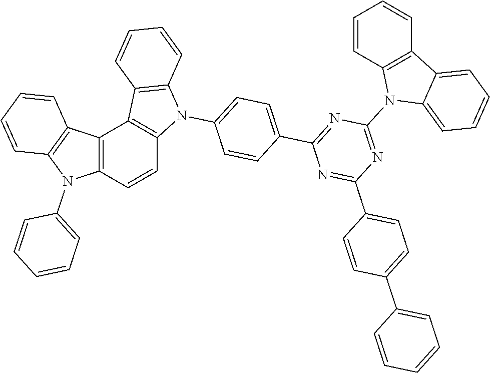 [SMILES: c1ccc(-c2ccc(-c3nc(-c4ccc(-n5c6ccccc6c6c7c8ccccc8n(-c8ccccc8)c7ccc65)cc4)nc(-n4c5ccccc5c5ccccc54)n3)cc2)cc1] | 1019 |
| 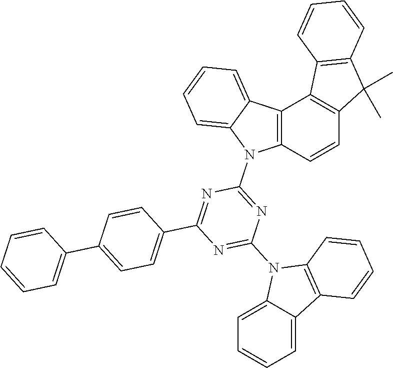 [SMILES: CC1(C)c2ccccc2-c2c1ccc1c2c2ccccc2n1-c1nc(-c2ccc(-c3ccccc3)cc2)nc(-n2c3ccccc3c3ccccc32)n1] | 1020 |
| 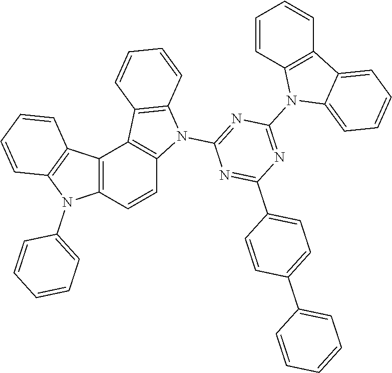 [SMILES: c1ccc(-c2ccc(-c3nc(-n4c5ccccc5c5ccccc54)nc(-n4c5ccccc5c5c6c7ccccc7n(-c7ccccc7)c6ccc54)n3)cc2)cc1] | 1021 |
| 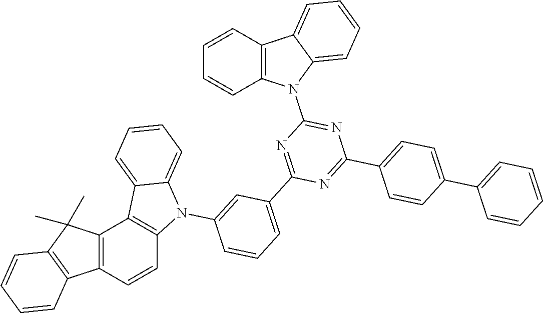 [SMILES: CC1(C)c2ccccc2-c2ccc3c(c21)c1ccccc1n3-c1cccc(-c2nc(-c3ccc(-c4ccccc4)cc3)nc(-n3c4ccccc4c4ccccc43)n2)c1] | 1022 |
| 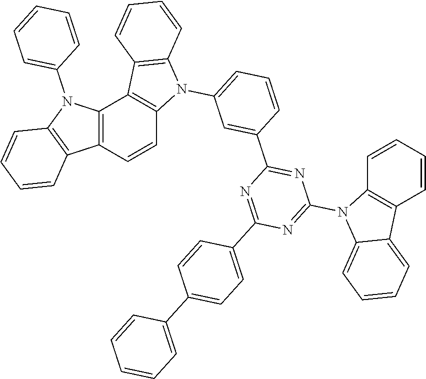 [SMILES: c1ccc(-c2ccc(-c3nc(-c4cccc(-n5c6ccccc6c6c5ccc5c7ccccc7n(-c7ccccc7)c56)c4)nc(-n4c5ccccc5c5ccccc54)n3)cc2)cc1] | 1023 |
| 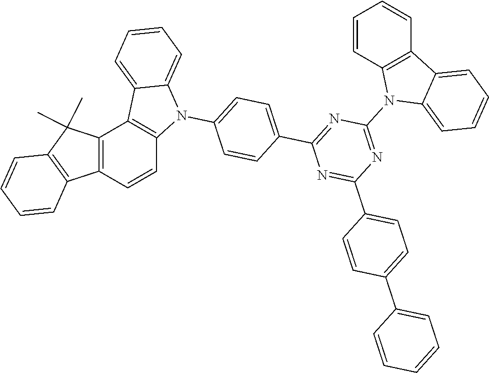 [SMILES: CC1(C)c2ccccc2-c2ccc3c(c21)c1ccccc1n3-c1ccc(-c2nc(-c3ccc(-c4ccccc4)cc3)nc(-n3c4ccccc4c4ccccc43)n2)cc1] | 1024 |
| 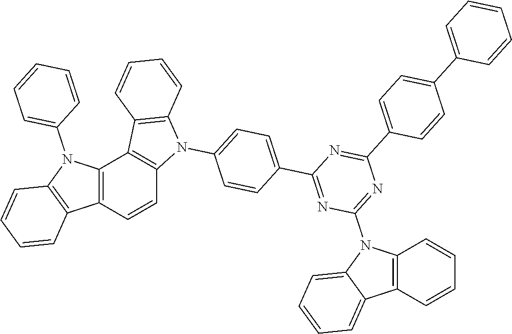 [SMILES: c1ccc(-c2ccc(-c3nc(-c4ccc(-n5c6ccccc6c6c5ccc5c7ccccc7n(-c7ccccc7)c56)cc4)nc(-n4c5ccccc5c5ccccc54)n3)cc2)cc1] | 1025 |
| 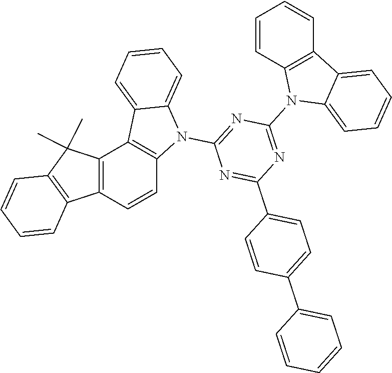 [SMILES: CC1(C)c2ccccc2-c2ccc3c(c21)c1ccccc1n3-c1nc(-c2ccc(-c3ccccc3)cc2)nc(-n2c3ccccc3c3ccccc32)n1] | 1026 |
| 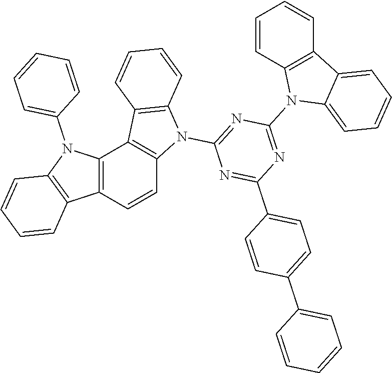 [SMILES: c1ccc(-c2ccc(-c3nc(-n4c5ccccc5c5ccccc54)nc(-n4c5ccccc5c5c4ccc4c6ccccc6n(-c6ccccc6)c45)n3)cc2)cc1] | 1027 |
| 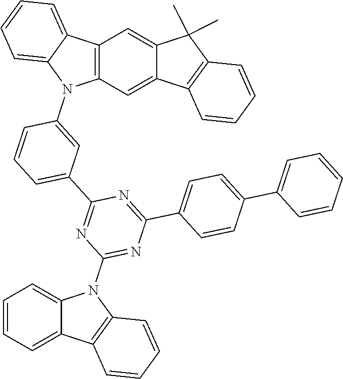 [SMILES: CC1(C)c2ccccc2-c2cc3c(cc21)c1ccccc1n3-c1cccc(-c2nc(-c3ccc(-c4ccccc4)cc3)nc(-n3c4ccccc4c4ccccc43)n2)c1] | 1028 |
| 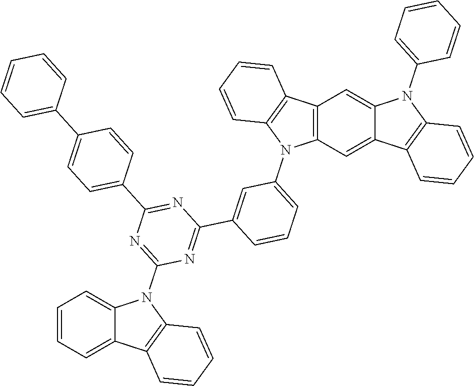 [SMILES: c1ccc(-c2ccc(-c3nc(-c4cccc(-n5c6ccccc6c6cc7c(cc65)c5ccccc5n7-c5ccccc5)c4)nc(-n4c5ccccc5c5ccccc54)n3)cc2)cc1] | 1029 |
| 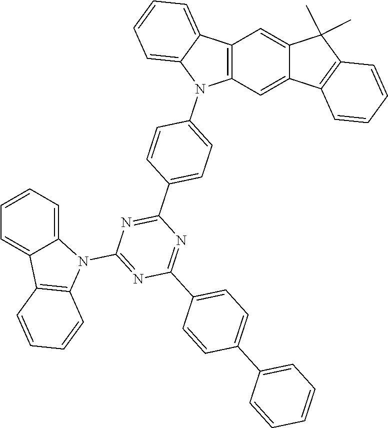 [SMILES: CC1(C)c2ccccc2-c2cc3c(cc21)c1ccccc1n3-c1ccc(-c2nc(-c3ccc(-c4ccccc4)cc3)nc(-n3c4ccccc4c4ccccc43)n2)cc1] | 1030 |
| 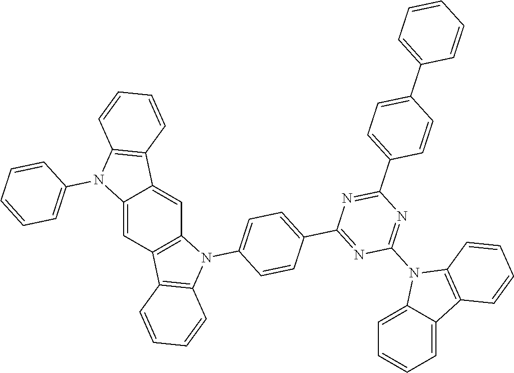 [SMILES: c1ccc(-c2ccc(-c3nc(-c4ccc(-n5c6ccccc6c6cc7c(cc65)c5ccccc5n7-c5ccccc5)cc4)nc(-n4c5ccccc5c5ccccc54)n3)cc2)cc1] | 1031 |
| 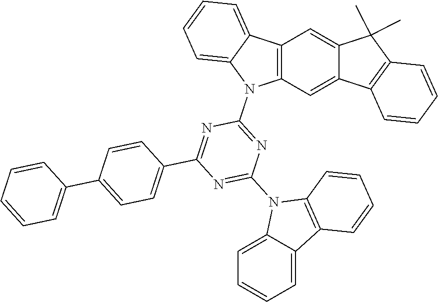 [SMILES: CC1(C)c2ccccc2-c2cc3c(cc21)c1ccccc1n3-c1nc(-c2ccc(-c3ccccc3)cc2)nc(-n2c3ccccc3c3ccccc32)n1] | 1032 |
| 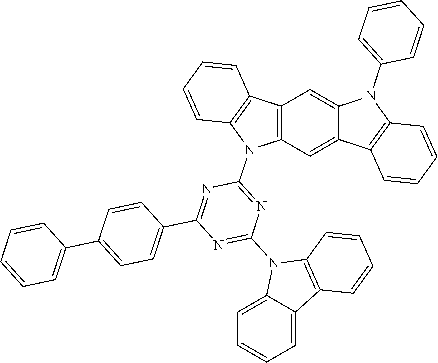 [SMILES: c1ccc(-c2ccc(-c3nc(-n4c5ccccc5c5ccccc54)nc(-n4c5ccccc5c5cc6c(cc54)c4ccccc4n6-c4ccccc4)n3)cc2)cc1] | 1033 |
| 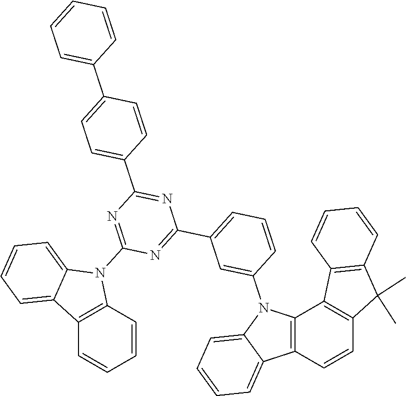 [SMILES: CC1(C)c2ccccc2-c2c1ccc1c3ccccc3n(-c3cccc(-c4nc(-c5ccc(-c6ccccc6)cc5)nc(-n5c6ccccc6c6ccccc65)n4)c3)c21] | 1034 |
| 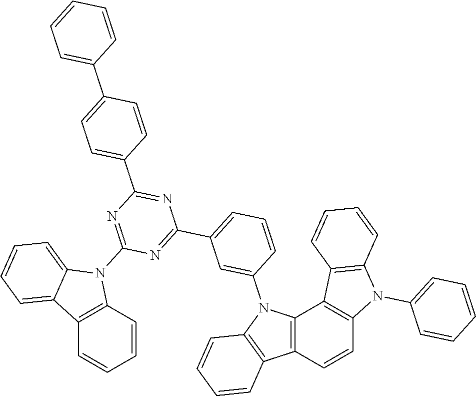 [SMILES: c1ccc(-c2ccc(-c3nc(-c4cccc(-n5c6ccccc6c6ccc7c(c8ccccc8n7-c7ccccc7)c65)c4)nc(-n4c5ccccc5c5ccccc54)n3)cc2)cc1] | 1035 |
| 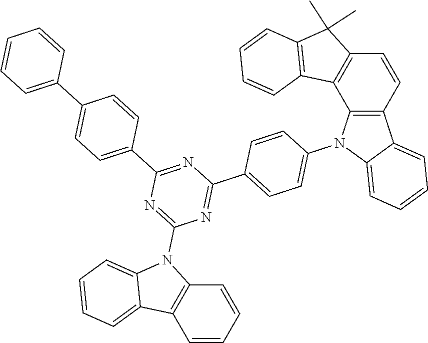 [SMILES: CC1(C)c2ccccc2-c2c1ccc1c3ccccc3n(-c3ccc(-c4nc(-c5ccc(-c6ccccc6)cc5)nc(-n5c6ccccc6c6ccccc65)n4)cc3)c21] | 1036 |
| 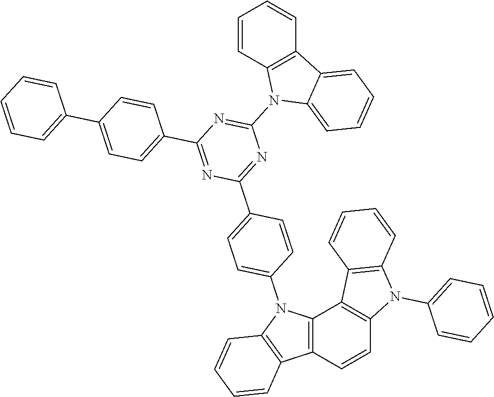 [SMILES: c1ccc(-c2ccc(-c3nc(-c4ccc(-n5c6ccccc6c6ccc7c(c8ccccc8n7-c7ccccc7)c65)cc4)nc(-n4c5ccccc5c5ccccc54)n3)cc2)cc1] | 1037 |
| 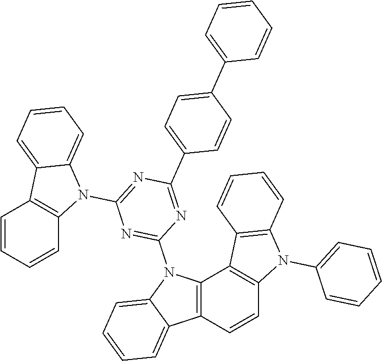 [SMILES: c1ccc(-c2ccc(-c3nc(-n4c5ccccc5c5ccccc54)nc(-n4c5ccccc5c5ccc6c(c7ccccc7n6-c6ccccc6)c54)n3)cc2)cc1] | 1038 |
| 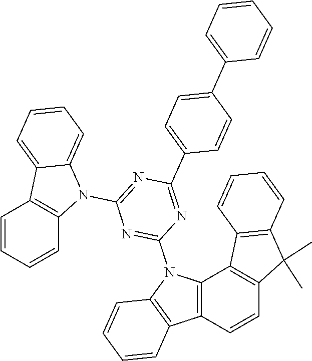 [SMILES: CC1(C)c2ccccc2-c2c1ccc1c3ccccc3n(-c3nc(-c4ccc(-c5ccccc5)cc4)nc(-n4c5ccccc5c5ccccc54)n3)c21] | 1039 |
| 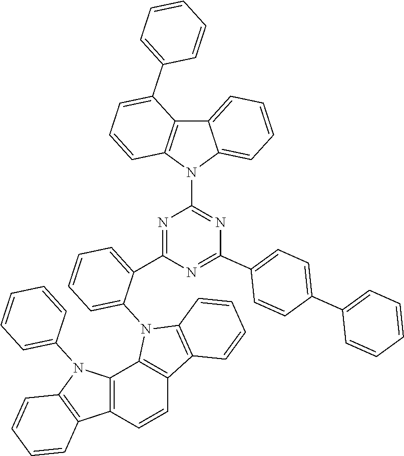 [SMILES: c1ccc(-c2ccc(-c3nc(-c4ccccc4-n4c5ccccc5c5ccc6c7ccccc7n(-c7ccccc7)c6c54)nc(-n4c5ccccc5c5c(-c6ccccc6)cccc54)n3)cc2)cc1] | 1040 |
| 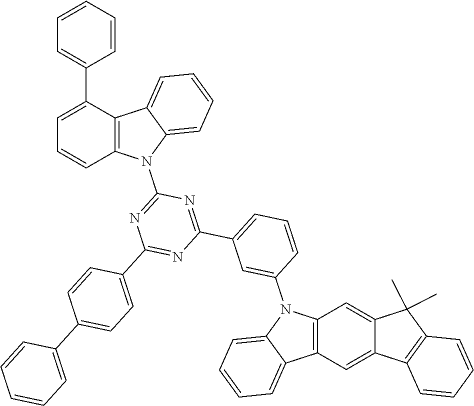 [SMILES: CC1(C)c2ccccc2-c2cc3c4ccccc4n(-c4cccc(-c5nc(-c6ccc(-c7ccccc7)cc6)nc(-n6c7ccccc7c7c(-c8ccccc8)cccc76)n5)c4)c3cc21] | 1041 |
| 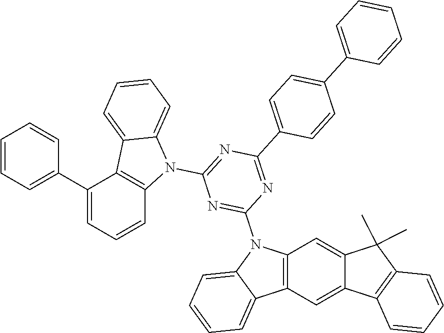 [SMILES: CC1(C)c2ccccc2-c2cc3c4ccccc4n(-c4nc(-c5ccc(-c6ccccc6)cc5)nc(-n5c6ccccc6c6c(-c7ccccc7)cccc65)n4)c3cc21] | 1042 |
| 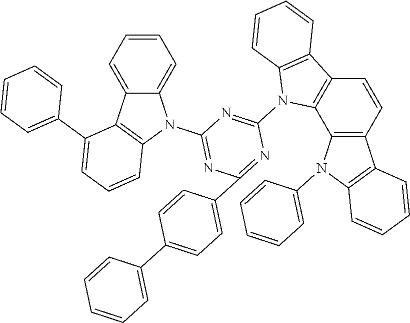 [SMILES: c1ccc(-c2ccc(-c3nc(-n4c5ccccc5c5c(-c6ccccc6)cccc54)nc(-n4c5ccccc5c5ccc6c7ccccc7n(-c7ccccc7)c6c54)n3)cc2)cc1] | 1043 |
| 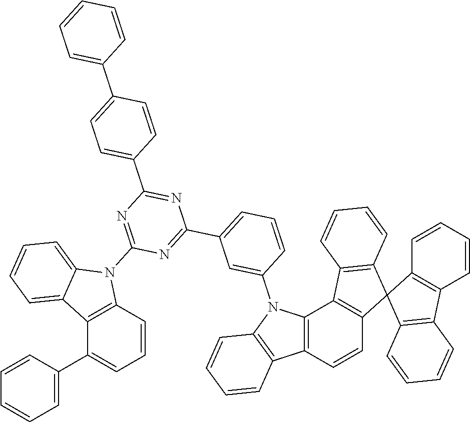 [SMILES: c1ccc(-c2ccc(-c3nc(-c4cccc(-n5c6ccccc6c6ccc7c(c65)-c5ccccc5C75c6ccccc6-c6ccccc65)c4)nc(-n4c5ccccc5c5c(-c6ccccc6)cccc54)n3)cc2)cc1] | 1044 |
| 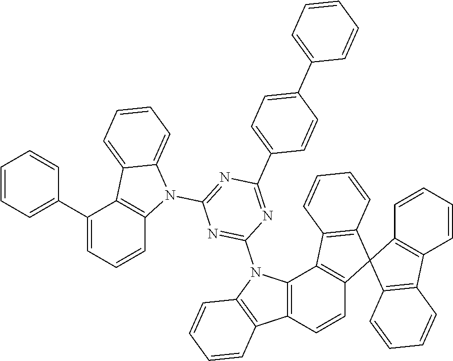 [SMILES: c1ccc(-c2ccc(-c3nc(-n4c5ccccc5c5c(-c6ccccc6)cccc54)nc(-n4c5ccccc5c5ccc6c(c54)-c4ccccc4C64c5ccccc5-c5ccccc54)n3)cc2)cc1] | 1045 |
| 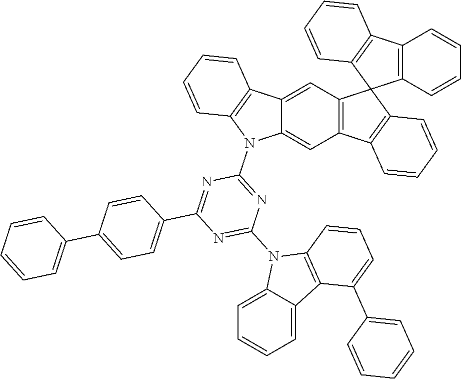 [SMILES: c1ccc(-c2ccc(-c3nc(-n4c5ccccc5c5cc6c(cc54)-c4ccccc4C64c5ccccc5-c5ccccc54)nc(-n4c5ccccc5c5c(-c6ccccc6)cccc54)n3)cc2)cc1] | 1046 |
| 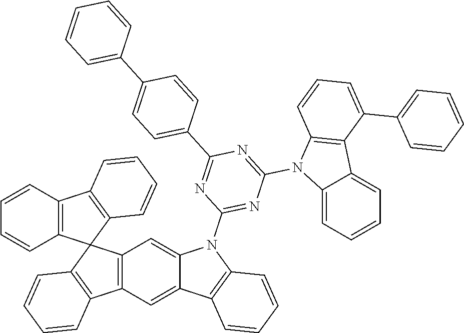 [SMILES: c1ccc(-c2ccc(-c3nc(-n4c5ccccc5c5cc6c(cc54)C4(c5ccccc5-c5ccccc54)c4ccccc4-6)nc(-n4c5ccccc5c5c(-c6ccccc6)cccc54)n3)cc2)cc1] | 1047 |
| 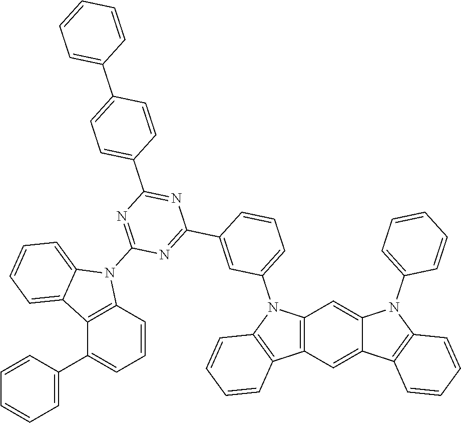 [SMILES: c1ccc(-c2ccc(-c3nc(-c4cccc(-n5c6ccccc6c6cc7c8ccccc8n(-c8ccccc8)c7cc65)c4)nc(-n4c5ccccc5c5c(-c6ccccc6)cccc54)n3)cc2)cc1] | 1048 |
| 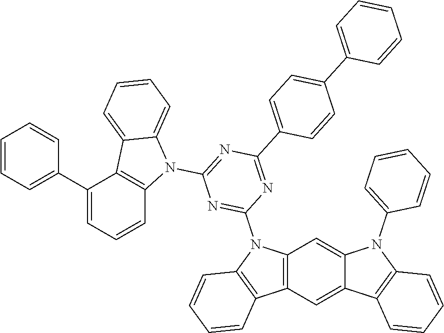 | 1049 |
| 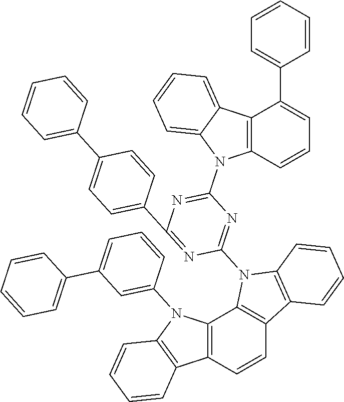 [SMILES: c1ccc(-c2ccc(-c3nc(-n4c5ccccc5c5c(-c6ccccc6)cccc54)nc(-n4c5ccccc5c5ccc6c7ccccc7n(-c7cccc(-c8ccccc8)c7)c6c54)n3)cc2)cc1] | 1050 |
| 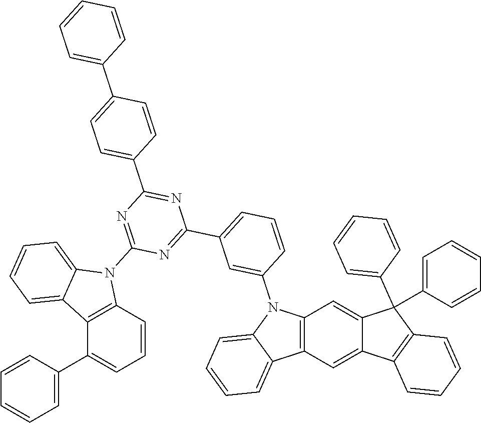 [SMILES: c1ccc(-c2ccc(-c3nc(-c4cccc(-n5c6ccccc6c6cc7c(cc65)C(c5ccccc5)(c5ccccc5)c5ccccc5-7)c4)nc(-n4c5ccccc5c5c(-c6ccccc6)cccc54)n3)cc2)cc1] | 1051 |
| 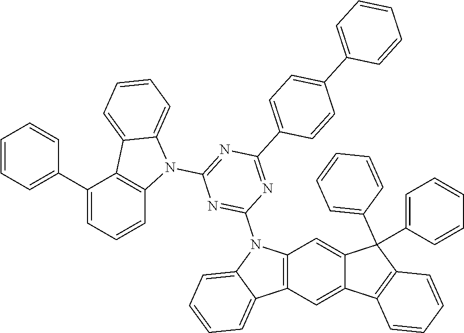 | 1052 |
| 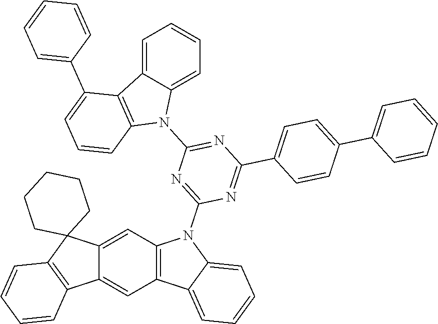 [SMILES: c1ccc(-c2ccc(-c3nc(-n4c5ccccc5c5cc6c(cc54)C4(CCCCC4)c4ccccc4-6)nc(-n4c5ccccc5c5c(-c6ccccc6)cccc54)n3)cc2)cc1] | 1053 |
| 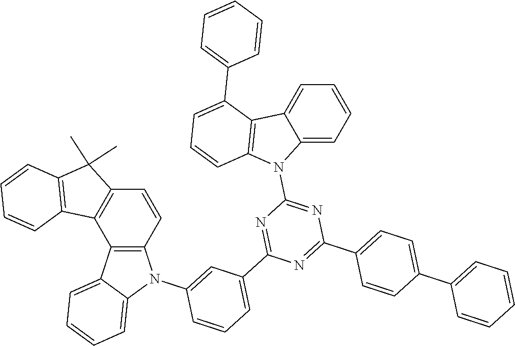 [SMILES: CC1(C)c2ccccc2-c2c1ccc1c2c2ccccc2n1-c1cccc(-c2nc(-c3ccc(-c4ccccc4)cc3)nc(-n3c4ccccc4c4c(-c5ccccc5)cccc43)n2)c1] | 1054 |
| 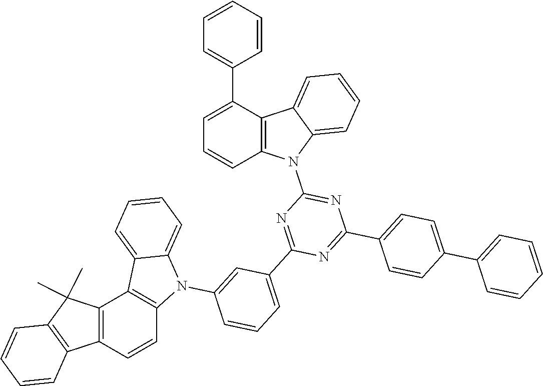 [SMILES: CC1(C)c2ccccc2-c2ccc3c(c21)c1ccccc1n3-c1cccc(-c2nc(-c3ccc(-c4ccccc4)cc3)nc(-n3c4ccccc4c4c(-c5ccccc5)cccc43)n2)c1] | 1055 |
| 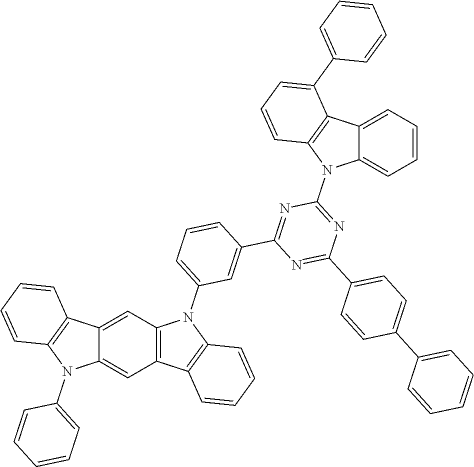 [SMILES: c1ccc(-c2ccc(-c3nc(-c4cccc(-n5c6ccccc6c6cc7c(cc65)c5ccccc5n7-c5ccccc5)c4)nc(-n4c5ccccc5c5c(-c6ccccc6)cccc54)n3)cc2)cc1] | 1056 |
| 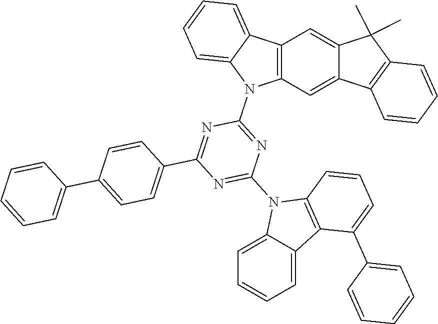 [SMILES: CC1(C)c2ccccc2-c2cc3c(cc21)c1ccccc1n3-c1nc(-c2ccc(-c3ccccc3)cc2)nc(-n2c3ccccc3c3c(-c4ccccc4)cccc32)n1] | 1057 |
| 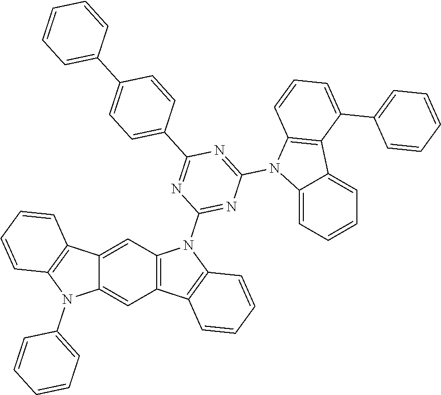 [SMILES: c1ccc(-c2ccc(-c3nc(-n4c5ccccc5c5cc6c(cc54)c4ccccc4n6-c4ccccc4)nc(-n4c5ccccc5c5c(-c6ccccc6)cccc54)n3)cc2)cc1] | 1058 |
| 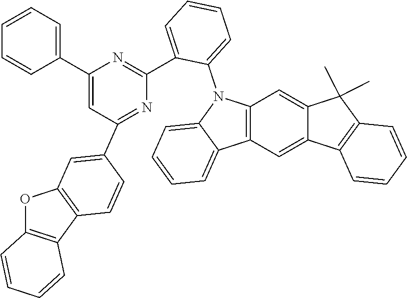 [SMILES: CC1(C)c2ccccc2-c2cc3c4ccccc4n(-c4ccccc4-c4nc(-c5ccccc5)cc(-c5ccc6c(c5)oc5ccccc56)n4)c3cc21] | 1059 |
| 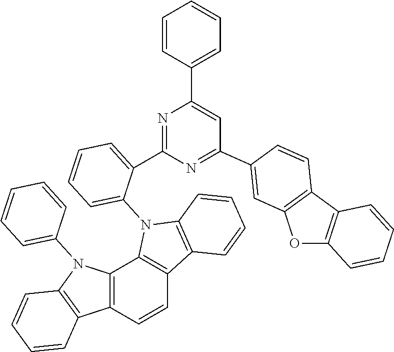 [SMILES: c1ccc(-c2cc(-c3ccc4c(c3)oc3ccccc34)nc(-c3ccccc3-n3c4ccccc4c4ccc5c6ccccc6n(-c6ccccc6)c5c43)n2)cc1] | 1060 |
| 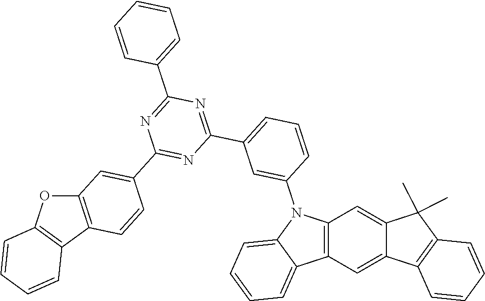 [SMILES: CC1(C)c2ccccc2-c2cc3c4ccccc4n(-c4cccc(-c5nc(-c6ccccc6)nc(-c6ccc7c(c6)oc6ccccc67)n5)c4)c3cc21] | 1061 |
| 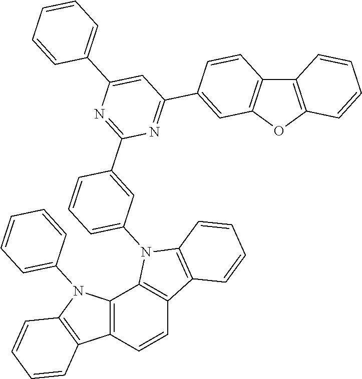 [SMILES: c1ccc(-c2cc(-c3ccc4c(c3)oc3ccccc34)nc(-c3cccc(-n4c5ccccc5c5ccc6c7ccccc7n(-c7ccccc7)c6c54)c3)n2)cc1] | 1062 |
| 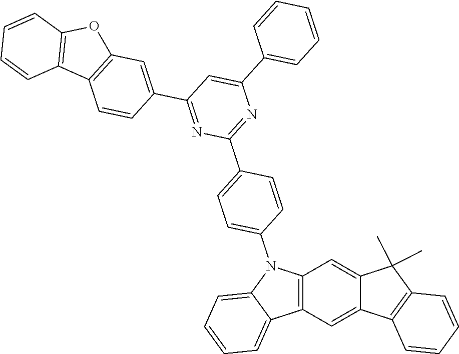 [SMILES: CC1(C)c2ccccc2-c2cc3c4ccccc4n(-c4ccc(-c5nc(-c6ccccc6)cc(-c6ccc7c(c6)oc6ccccc67)n5)cc4)c3cc21] | 1063 |
| 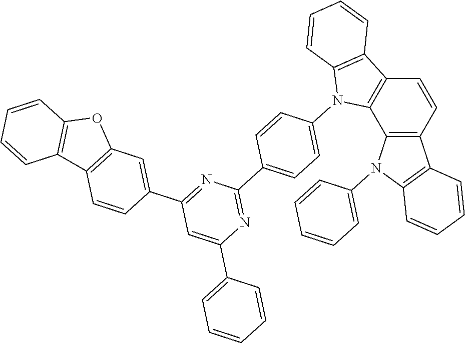 [SMILES: c1ccc(-c2cc(-c3ccc4c(c3)oc3ccccc34)nc(-c3ccc(-n4c5ccccc5c5ccc6c7ccccc7n(-c7ccccc7)c6c54)cc3)n2)cc1] | 1064 |
| 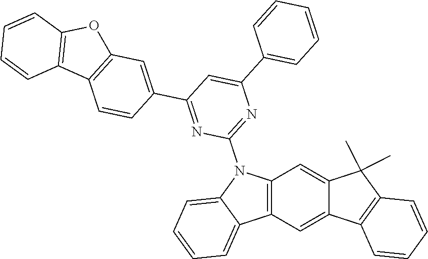 [SMILES: CC1(C)c2ccccc2-c2cc3c4ccccc4n(-c4nc(-c5ccccc5)cc(-c5ccc6c(c5)oc5ccccc56)n4)c3cc21] | 1065 |
| 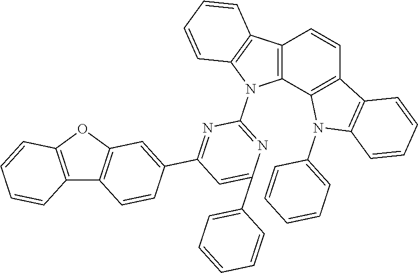 [SMILES: c1ccc(-c2cc(-c3ccc4c(c3)oc3ccccc34)nc(-n3c4ccccc4c4ccc5c6ccccc6n(-c6ccccc6)c5c43)n2)cc1] | 1066 |
| 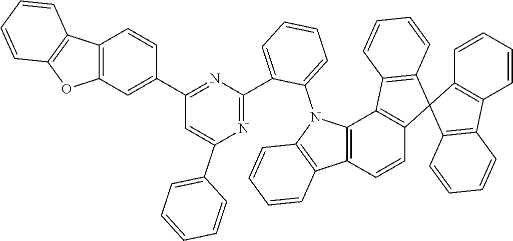 [SMILES: c1ccc(-c2cc(-c3ccc4c(c3)oc3ccccc34)nc(-c3ccccc3-n3c4ccccc4c4ccc5c(c43)-c3ccccc3C53c4ccccc4-c4ccccc43)n2)cc1] | 1067 |
| 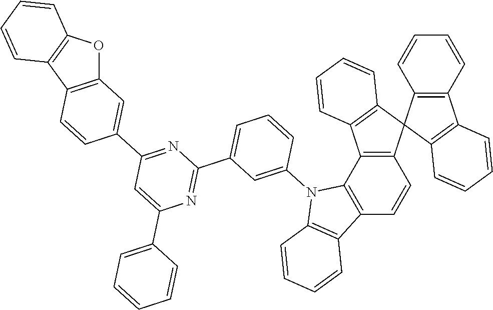 [SMILES: c1ccc(-c2cc(-c3ccc4c(c3)oc3ccccc34)nc(-c3cccc(-n4c5ccccc5c5ccc6c(c54)-c4ccccc4C64c5ccccc5-c5ccccc54)c3)n2)cc1] | 1068 |
| 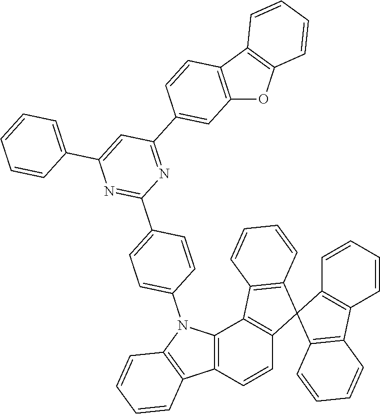 [SMILES: c1ccc(-c2cc(-c3ccc4c(c3)oc3ccccc34)nc(-c3ccc(-n4c5ccccc5c5ccc6c(c54)-c4ccccc4C64c5ccccc5-c5ccccc54)cc3)n2)cc1] | 1069 |
| 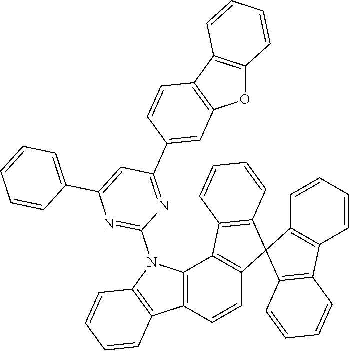 [SMILES: c1ccc(-c2cc(-c3ccc4c(c3)oc3ccccc34)nc(-n3c4ccccc4c4ccc5c(c43)-c3ccccc3C53c4ccccc4-c4ccccc43)n2)cc1] | 1070 |
| 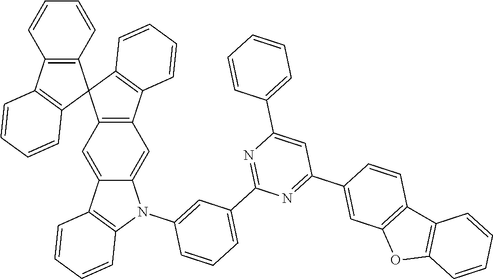 [SMILES: c1ccc(-c2cc(-c3ccc4c(c3)oc3ccccc34)nc(-c3cccc(-n4c5ccccc5c5cc6c(cc54)-c4ccccc4C64c5ccccc5-c5ccccc54)c3)n2)cc1] | 1071 |
| 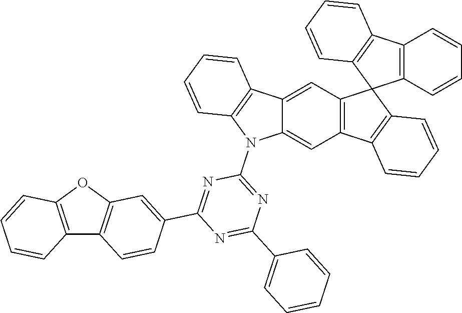 [SMILES: c1ccc(-c2nc(-c3ccc4c(c3)oc3ccccc34)nc(-n3c4ccccc4c4cc5c(cc43)-c3ccccc3C53c4ccccc4-c4ccccc43)n2)cc1] | 1072 |
| 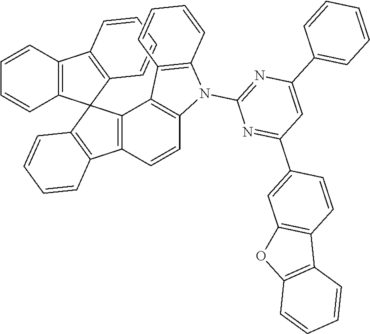 [SMILES: c1ccc(-c2cc(-c3ccc4c(c3)oc3ccccc34)nc(-n3c4ccccc4c4c5c(ccc43)-c3ccccc3C53c4ccccc4-c4ccccc43)n2)cc1] | 1073 |
| 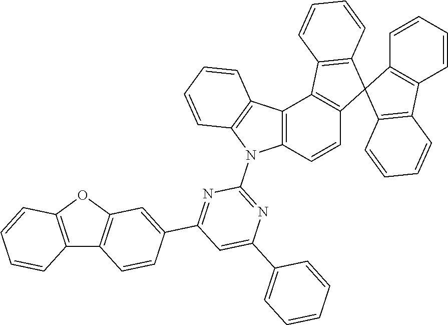 [SMILES: c1ccc(-c2cc(-c3ccc4c(c3)oc3ccccc34)nc(-n3c4ccccc4c4c5c(ccc43)C3(c4ccccc4-c4ccccc43)c3ccccc3-5)n2)cc1] | 1074 |
| 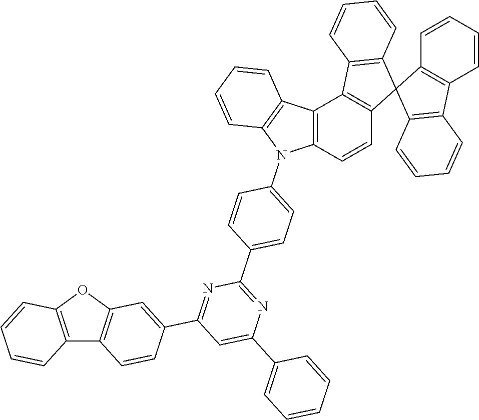 [SMILES: c1ccc(-c2cc(-c3ccc4c(c3)oc3ccccc34)nc(-c3ccc(-n4c5ccccc5c5c6c(ccc54)C4(c5ccccc5-c5ccccc54)c4ccccc4-6)cc3)n2)cc1] | 1075 |
| 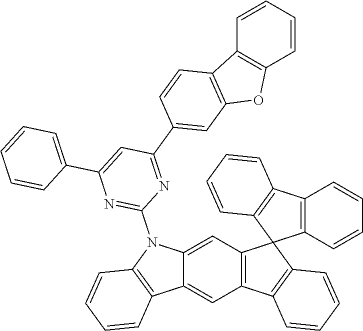 [SMILES: c1ccc(-c2cc(-c3ccc4c(c3)oc3ccccc34)nc(-n3c4ccccc4c4cc5c(cc43)C3(c4ccccc4-c4ccccc43)c3ccccc3-5)n2)cc1] | 1076 |
| 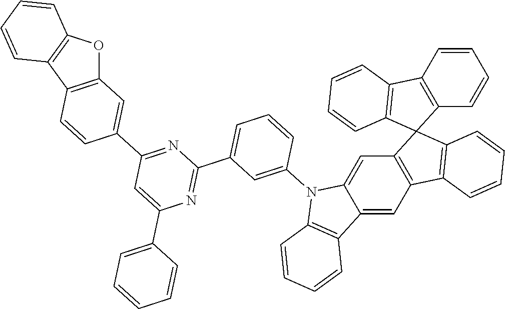 [SMILES: c1ccc(-c2cc(-c3ccc4c(c3)oc3ccccc34)nc(-c3cccc(-n4c5ccccc5c5cc6c(cc54)C4(c5ccccc5-c5ccccc54)c4ccccc4-6)c3)n2)cc1] | 1077 |
| 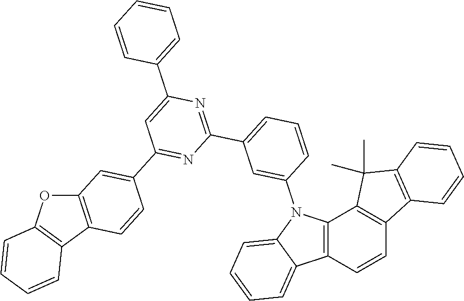 [SMILES: CC1(C)c2ccccc2-c2ccc3c4ccccc4n(-c4cccc(-c5nc(-c6ccccc6)cc(-c6ccc7c(c6)oc6ccccc67)n5)c4)c3c21] | 1078 |
| 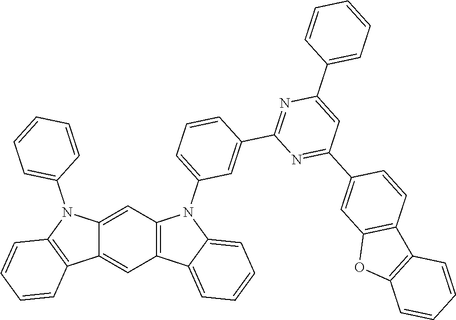 [SMILES: c1ccc(-c2cc(-c3ccc4c(c3)oc3ccccc34)nc(-c3cccc(-n4c5ccccc5c5cc6c7ccccc7n(-c7ccccc7)c6cc54)c3)n2)cc1] | 1079 |
| 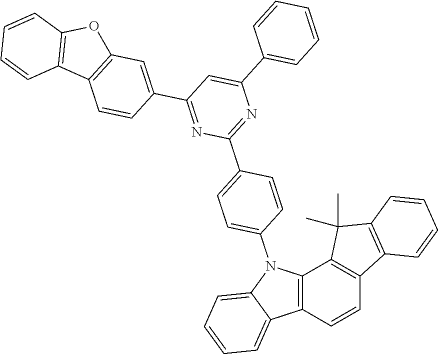 [SMILES: CC1(C)c2ccccc2-c2ccc3c4ccccc4n(-c4ccc(-c5nc(-c6ccccc6)cc(-c6ccc7c(c6)oc6ccccc67)n5)cc4)c3c21] | 1080 |
| 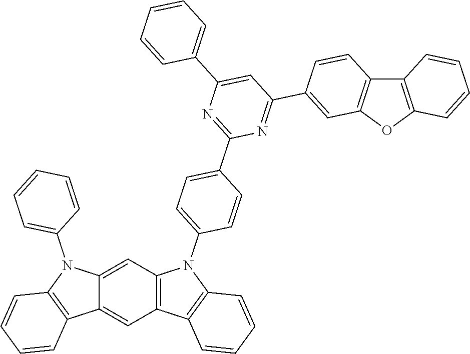 [SMILES: c1ccc(-c2cc(-c3ccc4c(c3)oc3ccccc34)nc(-c3ccc(-n4c5ccccc5c5cc6c7ccccc7n(-c7ccccc7)c6cc54)cc3)n2)cc1] | 1081 |
| 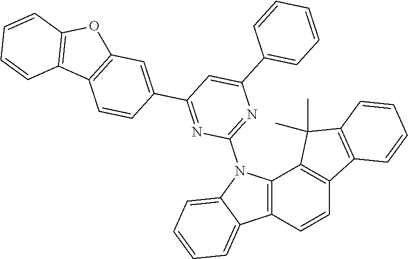 [SMILES: CC1(C)c2ccccc2-c2ccc3c4ccccc4n(-c4nc(-c5ccccc5)cc(-c5ccc6c(c5)oc5ccccc56)n4)c3c21] | 1082 |
| 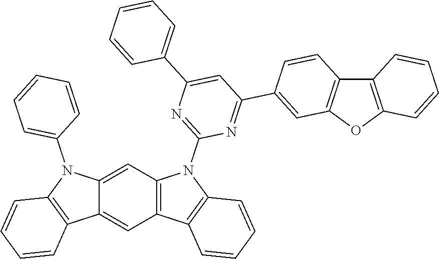 [SMILES: c1ccc(-c2cc(-c3ccc4c(c3)oc3ccccc34)nc(-n3c4ccccc4c4cc5c6ccccc6n(-c6ccccc6)c5cc43)n2)cc1] | 1083 |
| 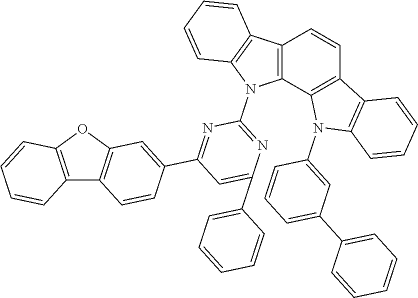 [SMILES: c1ccc(-c2cccc(-n3c4ccccc4c4ccc5c6ccccc6n(-c6nc(-c7ccccc7)cc(-c7ccc8c(c7)oc7ccccc78)n6)c5c43)c2)cc1] | 1084 |
| 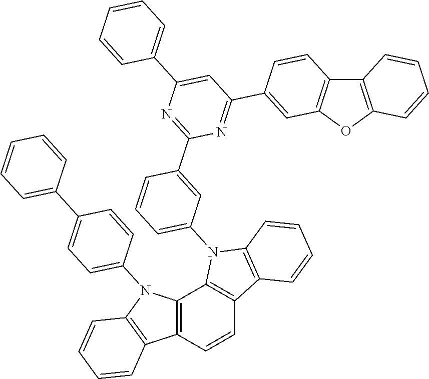 [SMILES: c1ccc(-c2ccc(-n3c4ccccc4c4ccc5c6ccccc6n(-c6cccc(-c7nc(-c8ccccc8)cc(-c8ccc9c(c8)oc8ccccc89)n7)c6)c5c43)cc2)cc1] | 1085 |
| 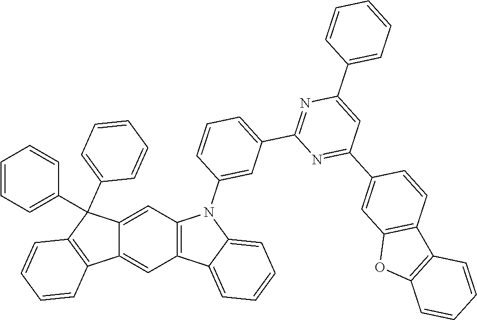 [SMILES: c1ccc(-c2cc(-c3ccc4c(c3)oc3ccccc34)nc(-c3cccc(-n4c5ccccc5c5cc6c(cc54)C(c4ccccc4)(c4ccccc4)c4ccccc4-6)c3)n2)cc1] | 1086 |
| 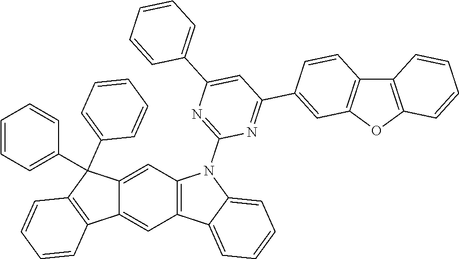 [SMILES: c1ccc(-c2cc(-c3ccc4c(c3)oc3ccccc34)nc(-n3c4ccccc4c4cc5c(cc43)C(c3ccccc3)(c3ccccc3)c3ccccc3-5)n2)cc1] | 1087 |
| 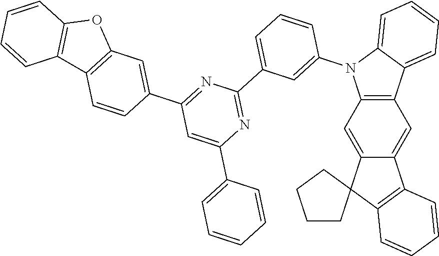 [SMILES: c1ccc(-c2cc(-c3ccc4c(c3)oc3ccccc34)nc(-c3cccc(-n4c5ccccc5c5cc6c(cc54)C4(CCCC4)c4ccccc4-6)c3)n2)cc1] | 1088 |
| 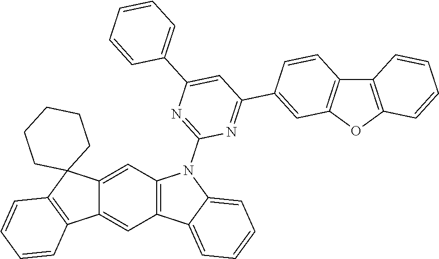 [SMILES: c1ccc(-c2cc(-c3ccc4c(c3)oc3ccccc34)nc(-n3c4ccccc4c4cc5c(cc43)C3(CCCCC3)c3ccccc3-5)n2)cc1] | 1089 |
| 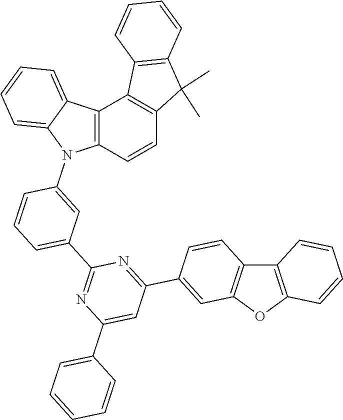 [SMILES: CC1(C)c2ccccc2-c2c1ccc1c2c2ccccc2n1-c1cccc(-c2nc(-c3ccccc3)cc(-c3ccc4c(c3)oc3ccccc34)n2)c1] | 1090 |
| 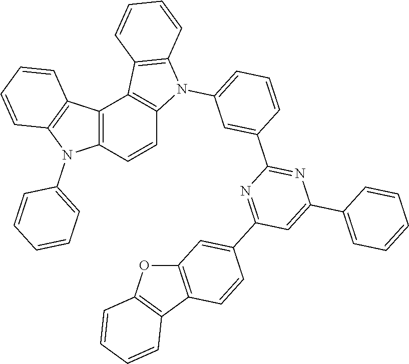 [SMILES: c1ccc(-c2cc(-c3ccc4c(c3)oc3ccccc34)nc(-c3cccc(-n4c5ccccc5c5c6c7ccccc7n(-c7ccccc7)c6ccc54)c3)n2)cc1] | 1091 |
| 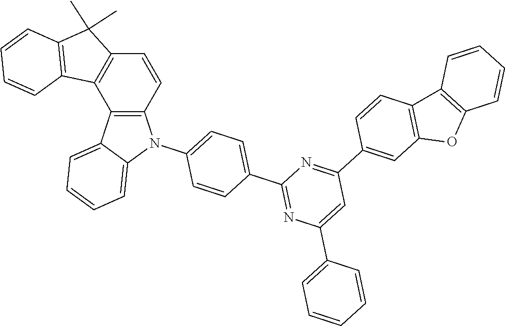 [SMILES: CC1(C)c2ccccc2-c2c1ccc1c2c2ccccc2n1-c1ccc(-c2nc(-c3ccccc3)cc(-c3ccc4c(c3)oc3ccccc34)n2)cc1] | 1092 |
| 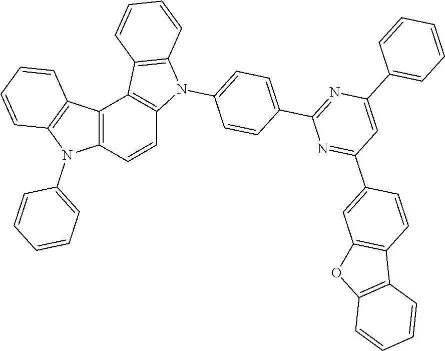 [SMILES: c1ccc(-c2cc(-c3ccc4c(c3)oc3ccccc34)nc(-c3ccc(-n4c5ccccc5c5c6c7ccccc7n(-c7ccccc7)c6ccc54)cc3)n2)cc1] | 1093 |
| 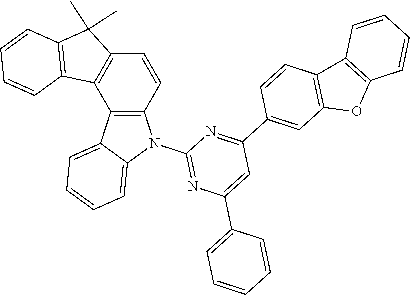 [SMILES: CC1(C)c2ccccc2-c2c1ccc1c2c2ccccc2n1-c1nc(-c2ccccc2)cc(-c2ccc3c(c2)oc2ccccc23)n1] | 1094 |
| 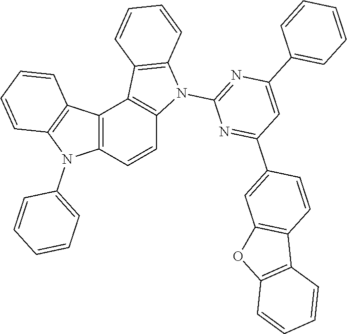 [SMILES: c1ccc(-c2cc(-c3ccc4c(c3)oc3ccccc34)nc(-n3c4ccccc4c4c5c6ccccc6n(-c6ccccc6)c5ccc43)n2)cc1] | 1095 |
| 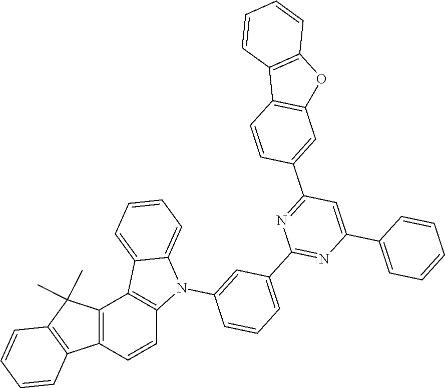 [SMILES: CC1(C)c2ccccc2-c2ccc3c(c21)c1ccccc1n3-c1cccc(-c2nc(-c3ccccc3)cc(-c3ccc4c(c3)oc3ccccc34)n2)c1] | 1096 |
| 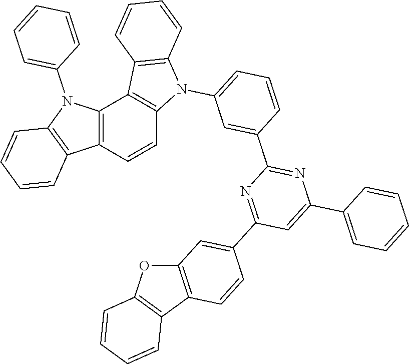 [SMILES: c1ccc(-c2cc(-c3ccc4c(c3)oc3ccccc34)nc(-c3cccc(-n4c5ccccc5c5c4ccc4c6ccccc6n(-c6ccccc6)c45)c3)n2)cc1] | 1097 |
| 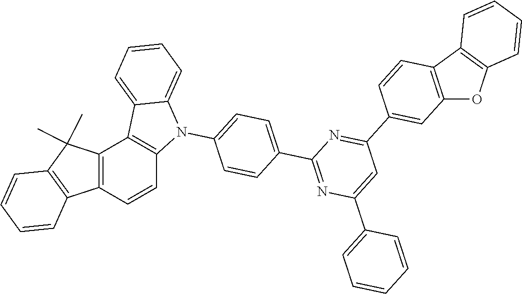 [SMILES: CC1(C)c2ccccc2-c2ccc3c(c21)c1ccccc1n3-c1ccc(-c2nc(-c3ccccc3)cc(-c3ccc4c(c3)oc3ccccc34)n2)cc1] | 1098 |
| 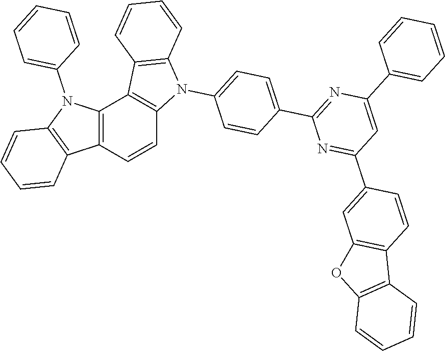 [SMILES: c1ccc(-c2cc(-c3ccc4c(c3)oc3ccccc34)nc(-c3ccc(-n4c5ccccc5c5c4ccc4c6ccccc6n(-c6ccccc6)c45)cc3)n2)cc1] | 1099 |
| 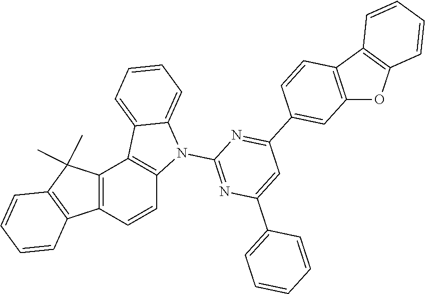 [SMILES: CC1(C)c2ccccc2-c2ccc3c(c21)c1ccccc1n3-c1nc(-c2ccccc2)cc(-c2ccc3c(c2)oc2ccccc23)n1] | 1100 |
| 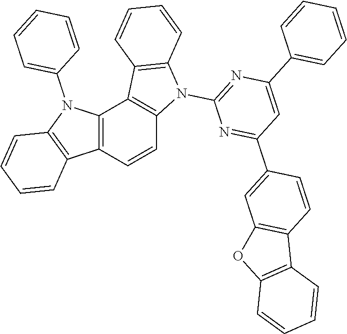 [SMILES: c1ccc(-c2cc(-c3ccc4c(c3)oc3ccccc34)nc(-n3c4ccccc4c4c3ccc3c5ccccc5n(-c5ccccc5)c34)n2)cc1] | 1101 |
| 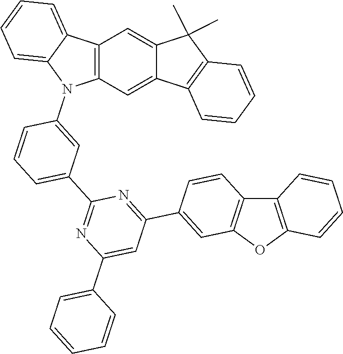 [SMILES: CC1(C)c2ccccc2-c2cc3c(cc21)c1ccccc1n3-c1cccc(-c2nc(-c3ccccc3)cc(-c3ccc4c(c3)oc3ccccc34)n2)c1] | 1102 |
| 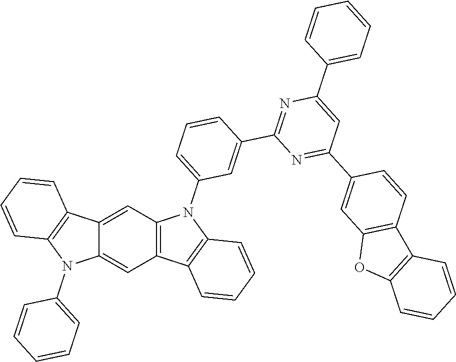 [SMILES: c1ccc(-c2cc(-c3ccc4c(c3)oc3ccccc34)nc(-c3cccc(-n4c5ccccc5c5cc6c(cc54)c4ccccc4n6-c4ccccc4)c3)n2)cc1] | 1103 |
| 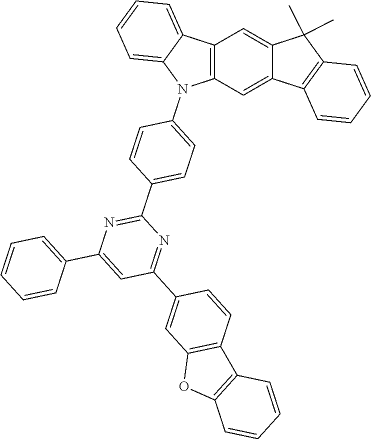 [SMILES: CC1(C)c2ccccc2-c2cc3c(cc21)c1ccccc1n3-c1ccc(-c2nc(-c3ccccc3)cc(-c3ccc4c(c3)oc3ccccc34)n2)cc1] | 1104 |
| 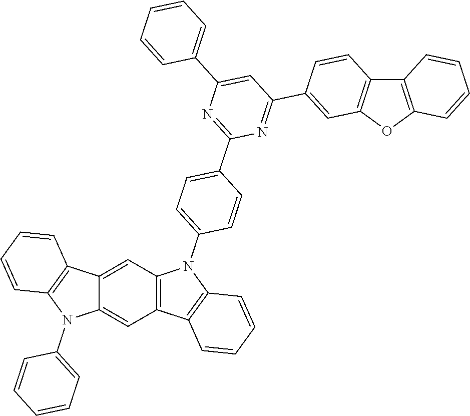 [SMILES: c1ccc(-c2cc(-c3ccc4c(c3)oc3ccccc34)nc(-c3ccc(-n4c5ccccc5c5cc6c(cc54)c4ccccc4n6-c4ccccc4)cc3)n2)cc1] | 1105 |
| 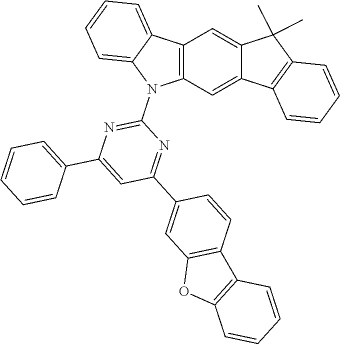 [SMILES: CC1(C)c2ccccc2-c2cc3c(cc21)c1ccccc1n3-c1nc(-c2ccccc2)cc(-c2ccc3c(c2)oc2ccccc23)n1] | 1106 |
| 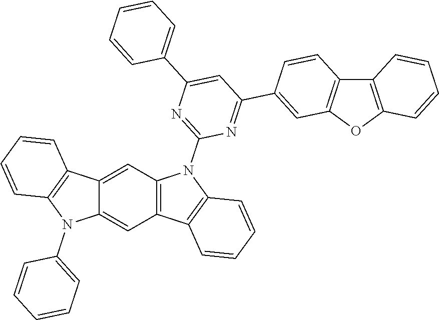 [SMILES: c1ccc(-c2cc(-c3ccc4c(c3)oc3ccccc34)nc(-n3c4ccccc4c4cc5c(cc43)c3ccccc3n5-c3ccccc3)n2)cc1] | 1107 |
| 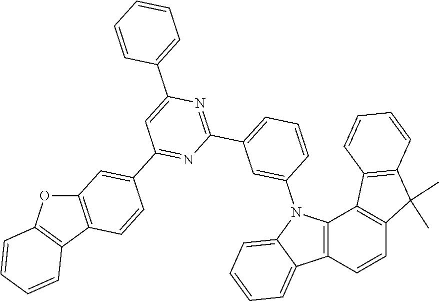 [SMILES: CC1(C)c2ccccc2-c2c1ccc1c3ccccc3n(-c3cccc(-c4nc(-c5ccccc5)cc(-c5ccc6c(c5)oc5ccccc56)n4)c3)c21] | 1108 |
| 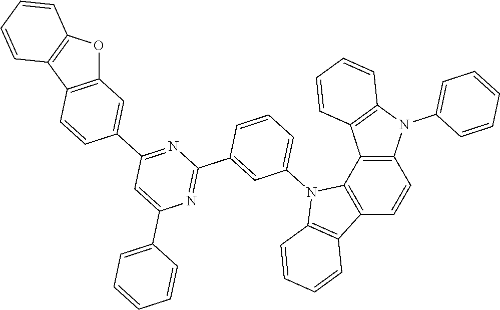 [SMILES: c1ccc(-c2cc(-c3ccc4c(c3)oc3ccccc34)nc(-c3cccc(-n4c5ccccc5c5ccc6c(c7ccccc7n6-c6ccccc6)c54)c3)n2)cc1] | 1109 |
| 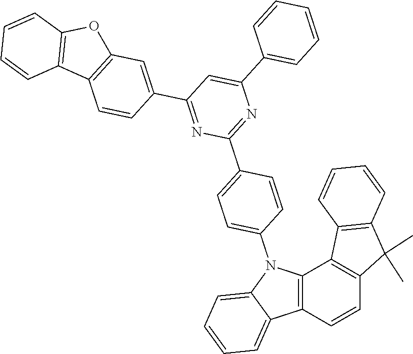 [SMILES: CC1(C)c2ccccc2-c2c1ccc1c3ccccc3n(-c3ccc(-c4nc(-c5ccccc5)cc(-c5ccc6c(c5)oc5ccccc56)n4)cc3)c21] | 1110 |
| 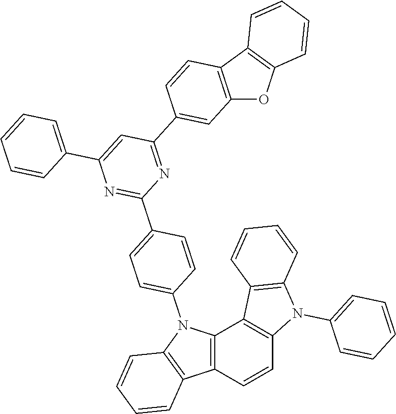 [SMILES: c1ccc(-c2cc(-c3ccc4c(c3)oc3ccccc34)nc(-c3ccc(-n4c5ccccc5c5ccc6c(c7ccccc7n6-c6ccccc6)c54)cc3)n2)cc1] | 1111 |
| 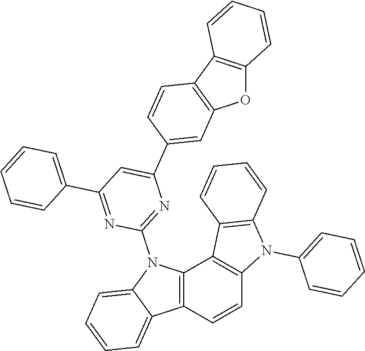 [SMILES: c1ccc(-c2cc(-c3ccc4c(c3)oc3ccccc34)nc(-n3c4ccccc4c4ccc5c(c6ccccc6n5-c5ccccc5)c43)n2)cc1] | 1112 |
| 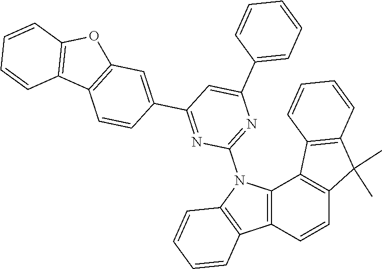 [SMILES: CC1(C)c2ccccc2-c2c1ccc1c3ccccc3n(-c3nc(-c4ccccc4)cc(-c4ccc5c(c4)oc4ccccc45)n3)c21] | 1113 |
| 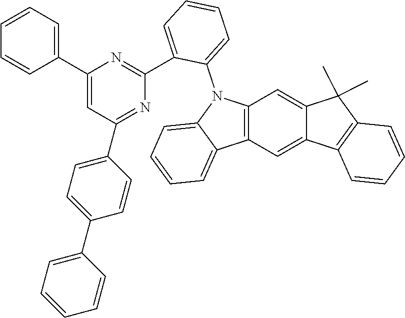 [SMILES: CC1(C)c2ccccc2-c2cc3c4ccccc4n(-c4ccccc4-c4nc(-c5ccccc5)cc(-c5ccc(-c6ccccc6)cc5)n4)c3cc21] | 1114 |
| 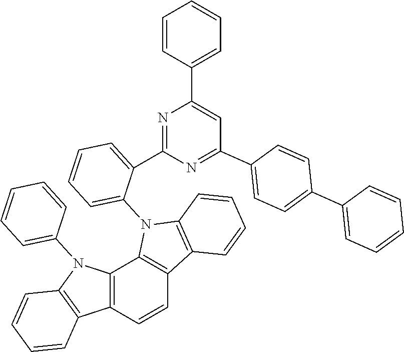 [SMILES: c1ccc(-c2ccc(-c3cc(-c4ccccc4)nc(-c4ccccc4-n4c5ccccc5c5ccc6c7ccccc7n(-c7ccccc7)c6c54)n3)cc2)cc1] | 1115 |
| 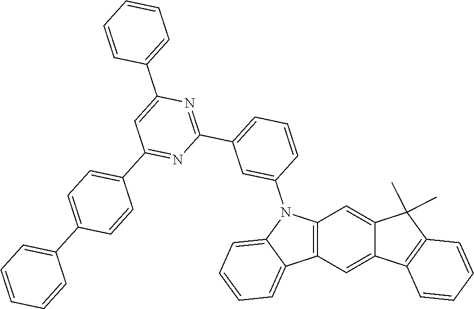 [SMILES: CC1(C)c2ccccc2-c2cc3c4ccccc4n(-c4cccc(-c5nc(-c6ccccc6)cc(-c6ccc(-c7ccccc7)cc6)n5)c4)c3cc21] | 1116 |
| 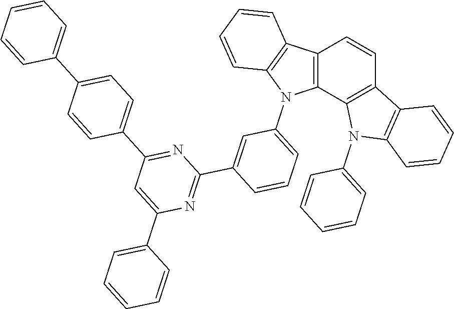 [SMILES: c1ccc(-c2ccc(-c3cc(-c4ccccc4)nc(-c4cccc(-n5c6ccccc6c6ccc7c8ccccc8n(-c8ccccc8)c7c65)c4)n3)cc2)cc1] | 1117 |
| 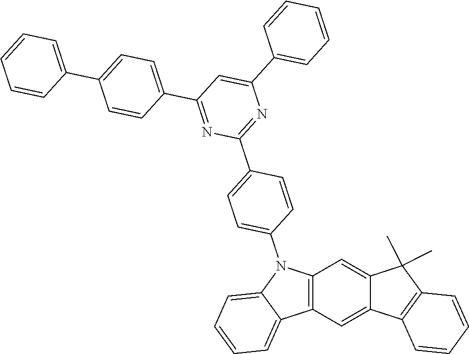 [SMILES: CC1(C)c2ccccc2-c2cc3c4ccccc4n(-c4ccc(-c5nc(-c6ccccc6)cc(-c6ccc(-c7ccccc7)cc6)n5)cc4)c3cc21] | 1118 |
| 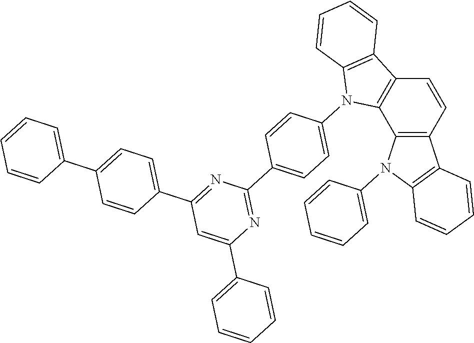 [SMILES: c1ccc(-c2ccc(-c3cc(-c4ccccc4)nc(-c4ccc(-n5c6ccccc6c6ccc7c8ccccc8n(-c8ccccc8)c7c65)cc4)n3)cc2)cc1] | 1119 |
| 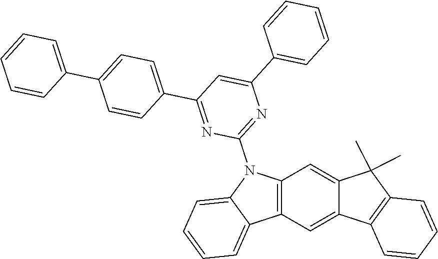 [SMILES: CC1(C)c2ccccc2-c2cc3c4ccccc4n(-c4nc(-c5ccccc5)cc(-c5ccc(-c6ccccc6)cc5)n4)c3cc21] | 1120 |
| 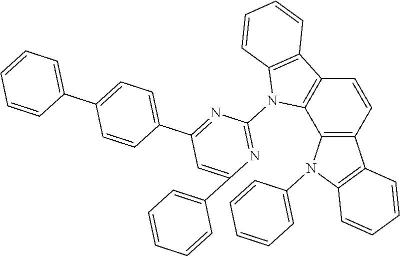 [SMILES: c1ccc(-c2ccc(-c3cc(-c4ccccc4)nc(-n4c5ccccc5c5ccc6c7ccccc7n(-c7ccccc7)c6c54)n3)cc2)cc1] | 1121 |
| 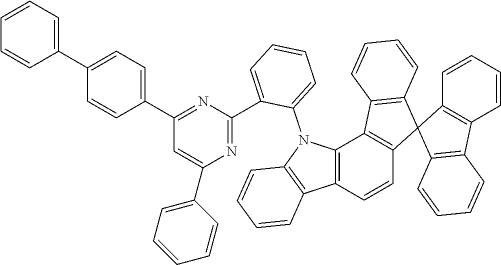 [SMILES: c1ccc(-c2ccc(-c3cc(-c4ccccc4)nc(-c4ccccc4-n4c5ccccc5c5ccc6c(c54)-c4ccccc4C64c5ccccc5-c5ccccc54)n3)cc2)cc1] | 1122 |
| 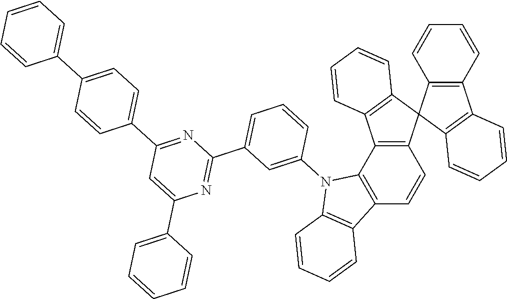 [SMILES: c1ccc(-c2ccc(-c3cc(-c4ccccc4)nc(-c4cccc(-n5c6ccccc6c6ccc7c(c65)-c5ccccc5C75c6ccccc6-c6ccccc65)c4)n3)cc2)cc1] | 1123 |
| 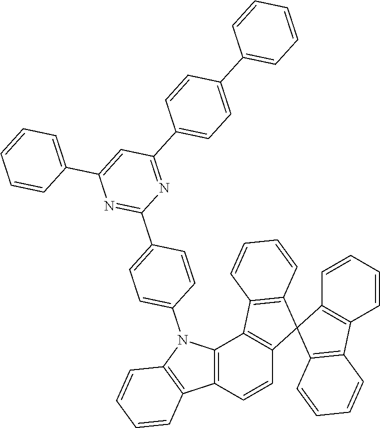 [SMILES: c1ccc(-c2ccc(-c3cc(-c4ccccc4)nc(-c4ccc(-n5c6ccccc6c6ccc7c(c65)-c5ccccc5C75c6ccccc6-c6ccccc65)cc4)n3)cc2)cc1] | 1124 |
| 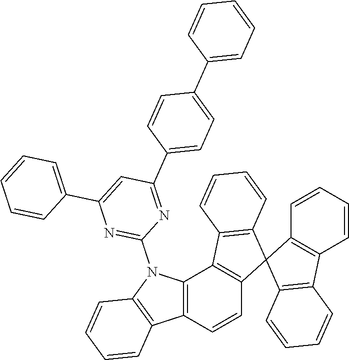 [SMILES: c1ccc(-c2ccc(-c3cc(-c4ccccc4)nc(-n4c5ccccc5c5ccc6c(c54)-c4ccccc4C64c5ccccc5-c5ccccc54)n3)cc2)cc1] | 1125 |
| 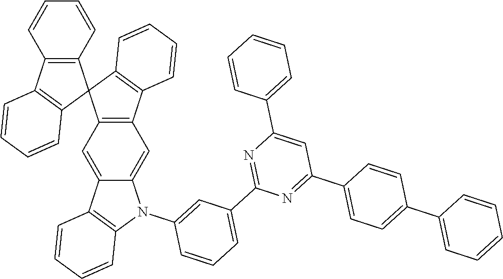 [SMILES: c1ccc(-c2ccc(-c3cc(-c4ccccc4)nc(-c4cccc(-n5c6ccccc6c6cc7c(cc65)-c5ccccc5C75c6ccccc6-c6ccccc65)c4)n3)cc2)cc1] | 1126 |
| 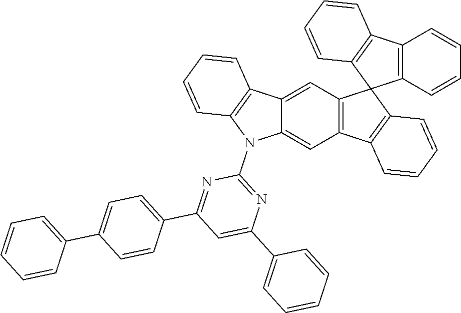 [SMILES: c1ccc(-c2ccc(-c3cc(-c4ccccc4)nc(-n4c5ccccc5c5cc6c(cc54)-c4ccccc4C64c5ccccc5-c5ccccc54)n3)cc2)cc1] | 1127 |
| 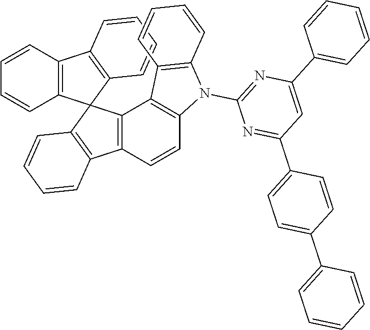 [SMILES: c1ccc(-c2ccc(-c3cc(-c4ccccc4)nc(-n4c5ccccc5c5c6c(ccc54)-c4ccccc4C64c5ccccc5-c5ccccc54)n3)cc2)cc1] | 1128 |
| 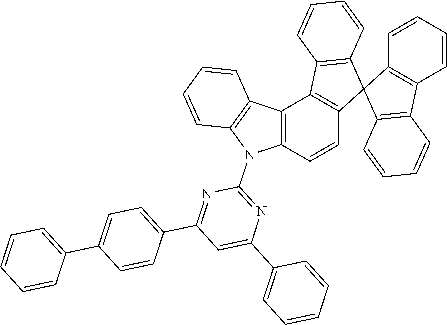 [SMILES: c1ccc(-c2ccc(-c3cc(-c4ccccc4)nc(-n4c5ccccc5c5c6c(ccc54)C4(c5ccccc5-c5ccccc54)c4ccccc4-6)n3)cc2)cc1] | 1129 |
| 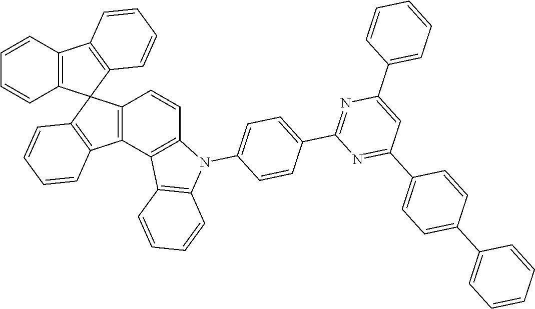 [SMILES: c1ccc(-c2ccc(-c3cc(-c4ccccc4)nc(-c4ccc(-n5c6ccccc6c6c7c(ccc65)C5(c6ccccc6-c6ccccc65)c5ccccc5-7)cc4)n3)cc2)cc1] | 1130 |
| 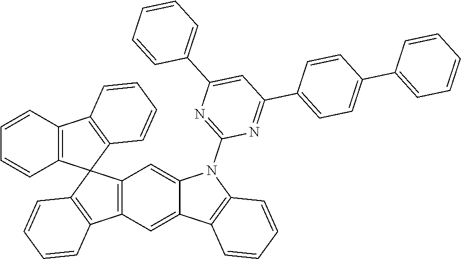 [SMILES: c1ccc(-c2ccc(-c3cc(-c4ccccc4)nc(-n4c5ccccc5c5cc6c(cc54)C4(c5ccccc5-c5ccccc54)c4ccccc4-6)n3)cc2)cc1] | 1131 |
| 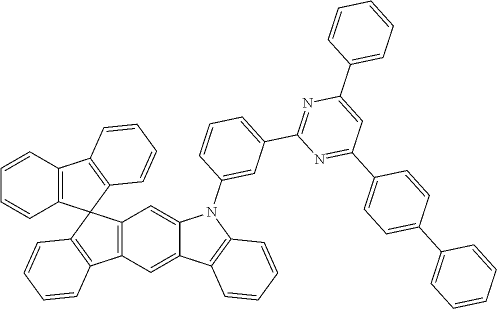 [SMILES: c1ccc(-c2ccc(-c3cc(-c4ccccc4)nc(-c4cccc(-n5c6ccccc6c6cc7c(cc65)C5(c6ccccc6-c6ccccc65)c5ccccc5-7)c4)n3)cc2)cc1] | 1132 |
| 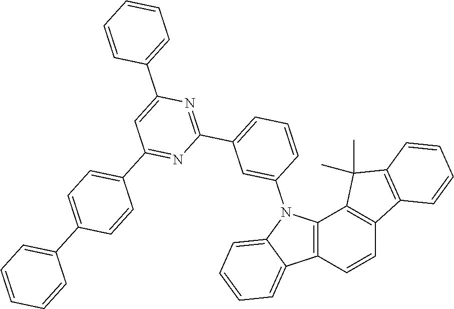 [SMILES: CC1(C)c2ccccc2-c2ccc3c4ccccc4n(-c4cccc(-c5nc(-c6ccccc6)cc(-c6ccc(-c7ccccc7)cc6)n5)c4)c3c21] | 1133 |
| 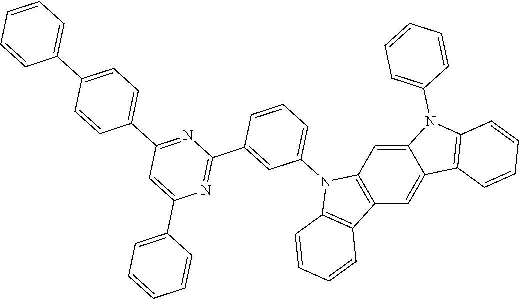 [SMILES: c1ccc(-c2ccc(-c3cc(-c4ccccc4)nc(-c4cccc(-n5c6ccccc6c6cc7c8ccccc8n(-c8ccccc8)c7cc65)c4)n3)cc2)cc1] | 1134 |
| 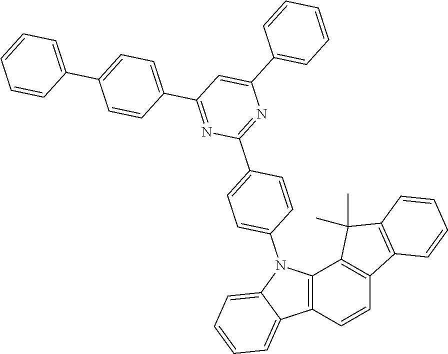 [SMILES: CC1(C)c2ccccc2-c2ccc3c4ccccc4n(-c4ccc(-c5nc(-c6ccccc6)cc(-c6ccc(-c7ccccc7)cc6)n5)cc4)c3c21] | 1135 |
| 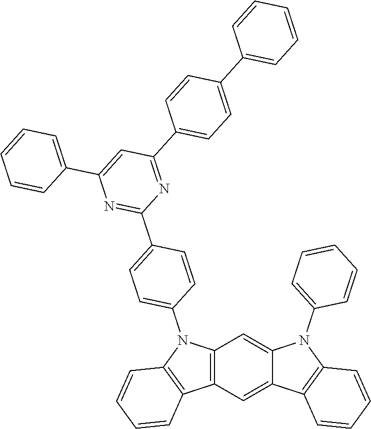 [SMILES: c1ccc(-c2ccc(-c3cc(-c4ccccc4)nc(-c4ccc(-n5c6ccccc6c6cc7c8ccccc8n(-c8ccccc8)c7cc65)cc4)n3)cc2)cc1] | 1136 |
| 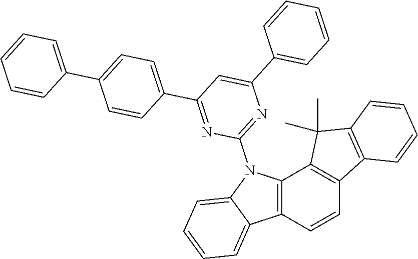 [SMILES: CC1(C)c2ccccc2-c2ccc3c4ccccc4n(-c4nc(-c5ccccc5)cc(-c5ccc(-c6ccccc6)cc5)n4)c3c21] | 1137 |
| 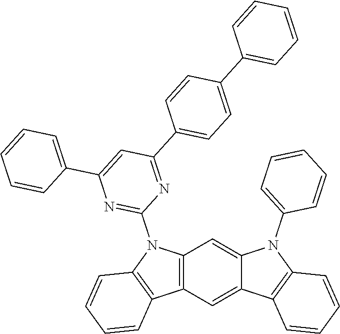 [SMILES: c1ccc(-c2ccc(-c3cc(-c4ccccc4)nc(-n4c5ccccc5c5cc6c7ccccc7n(-c7ccccc7)c6cc54)n3)cc2)cc1] | 1138 |
| 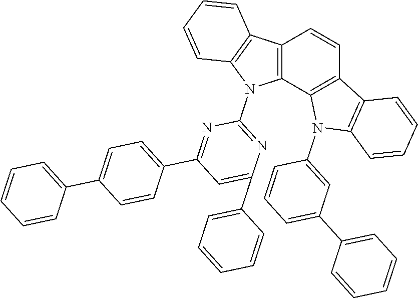 [SMILES: c1ccc(-c2ccc(-c3cc(-c4ccccc4)nc(-n4c5ccccc5c5ccc6c7ccccc7n(-c7cccc(-c8ccccc8)c7)c6c54)n3)cc2)cc1] | 1139 |
| 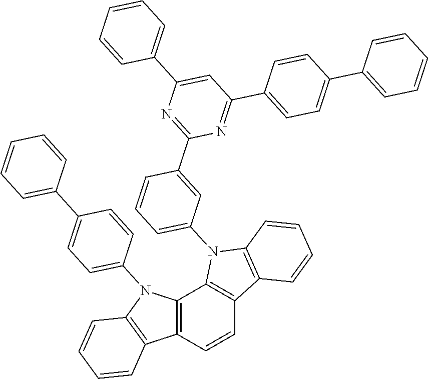 [SMILES: c1ccc(-c2ccc(-c3cc(-c4ccccc4)nc(-c4cccc(-n5c6ccccc6c6ccc7c8ccccc8n(-c8ccc(-c9ccccc9)cc8)c7c65)c4)n3)cc2)cc1] | 1140 |
| 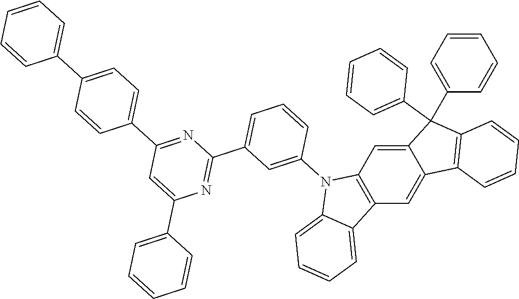 [SMILES: c1ccc(-c2ccc(-c3cc(-c4ccccc4)nc(-c4cccc(-n5c6ccccc6c6cc7c(cc65)C(c5ccccc5)(c5ccccc5)c5ccccc5-7)c4)n3)cc2)cc1] | 1141 |
| 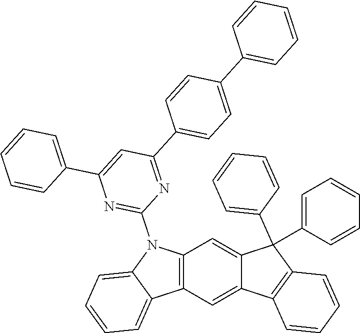 [SMILES: c1ccc(-c2ccc(-c3cc(-c4ccccc4)nc(-n4c5ccccc5c5cc6c(cc54)C(c4ccccc4)(c4ccccc4)c4ccccc4-6)n3)cc2)cc1] | 1142 |
| 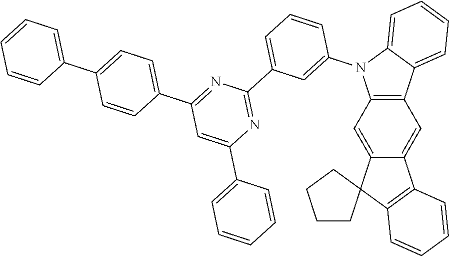 [SMILES: c1ccc(-c2ccc(-c3cc(-c4ccccc4)nc(-c4cccc(-n5c6ccccc6c6cc7c(cc65)C5(CCCC5)c5ccccc5-7)c4)n3)cc2)cc1] | 1143 |
| 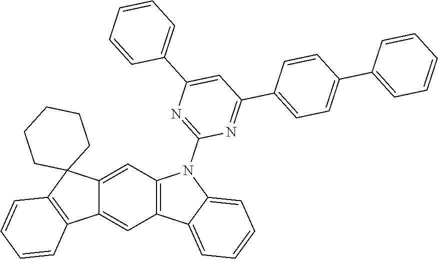 [SMILES: c1ccc(-c2ccc(-c3cc(-c4ccccc4)nc(-n4c5ccccc5c5cc6c(cc54)C4(CCCCC4)c4ccccc4-6)n3)cc2)cc1] | 1144 |
| 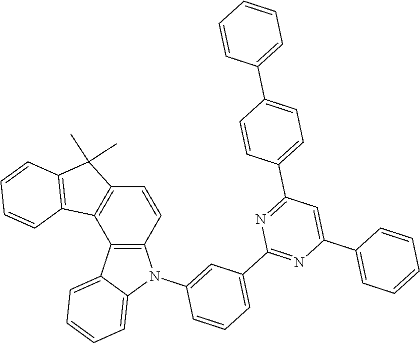 [SMILES: CC1(C)c2ccccc2-c2c1ccc1c2c2ccccc2n1-c1cccc(-c2nc(-c3ccccc3)cc(-c3ccc(-c4ccccc4)cc3)n2)c1] | 1145 |
| 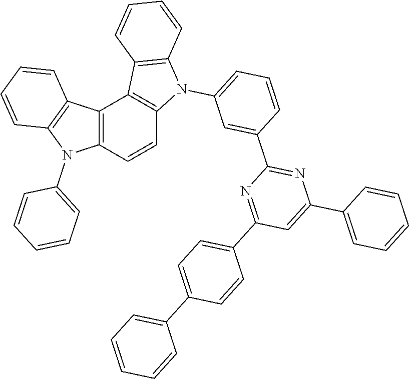 [SMILES: c1ccc(-c2ccc(-c3cc(-c4ccccc4)nc(-c4cccc(-n5c6ccccc6c6c7c8ccccc8n(-c8ccccc8)c7ccc65)c4)n3)cc2)cc1] | 1146 |
| 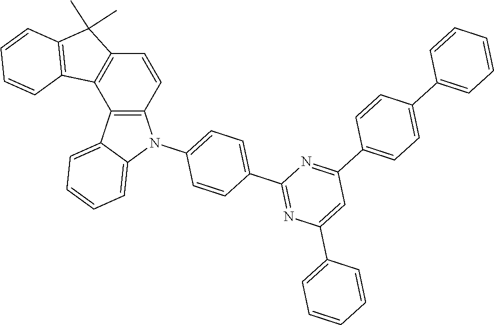 [SMILES: CC1(C)c2ccccc2-c2c1ccc1c2c2ccccc2n1-c1ccc(-c2nc(-c3ccccc3)cc(-c3ccc(-c4ccccc4)cc3)n2)cc1] | 1147 |
| 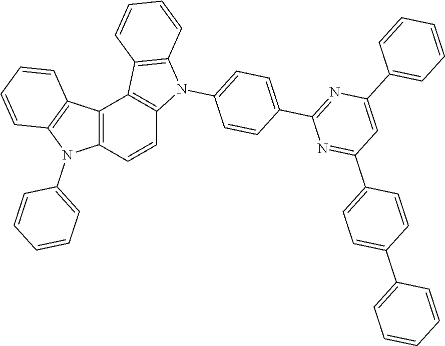 [SMILES: c1ccc(-c2ccc(-c3cc(-c4ccccc4)nc(-c4ccc(-n5c6ccccc6c6c7c8ccccc8n(-c8ccccc8)c7ccc65)cc4)n3)cc2)cc1] | 1148 |
| 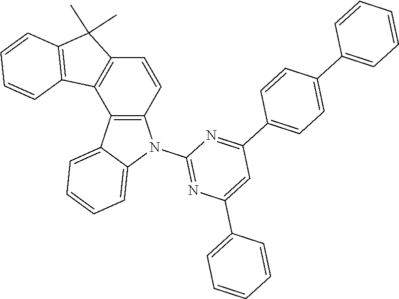 [SMILES: CC1(C)c2ccccc2-c2c1ccc1c2c2ccccc2n1-c1nc(-c2ccccc2)cc(-c2ccc(-c3ccccc3)cc2)n1] | 1149 |
| 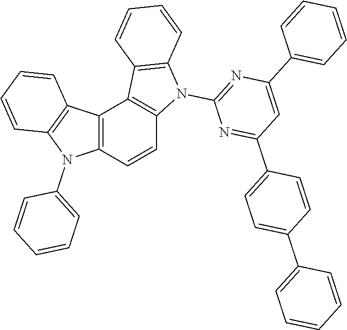 [SMILES: c1ccc(-c2ccc(-c3cc(-c4ccccc4)nc(-n4c5ccccc5c5c6c7ccccc7n(-c7ccccc7)c6ccc54)n3)cc2)cc1] | 1150 |
| 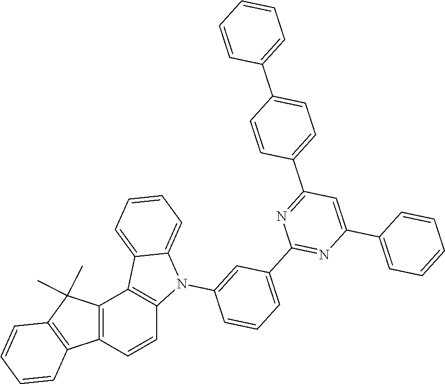 [SMILES: CC1(C)c2ccccc2-c2ccc3c(c21)c1ccccc1n3-c1cccc(-c2nc(-c3ccccc3)cc(-c3ccc(-c4ccccc4)cc3)n2)c1] | 1151 |
| 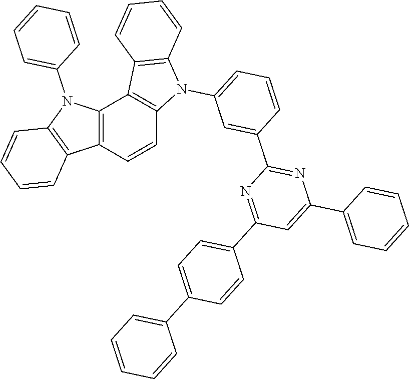 [SMILES: c1ccc(-c2ccc(-c3cc(-c4ccccc4)nc(-c4cccc(-n5c6ccccc6c6c5ccc5c7ccccc7n(-c7ccccc7)c56)c4)n3)cc2)cc1] | 1152 |
| 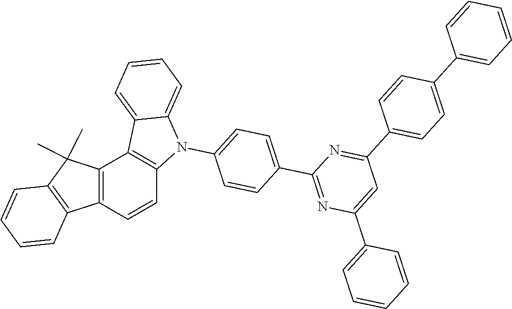 [SMILES: CC1(C)c2ccccc2-c2ccc3c(c21)c1ccccc1n3-c1ccc(-c2nc(-c3ccccc3)cc(-c3ccc(-c4ccccc4)cc3)n2)cc1] | 1153 |
| 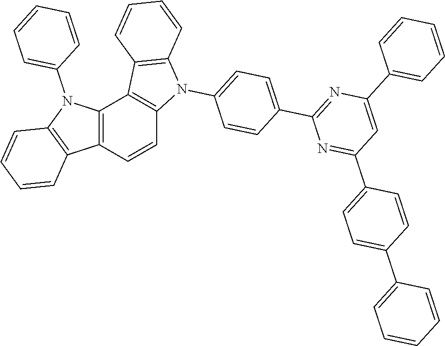 [SMILES: c1ccc(-c2ccc(-c3cc(-c4ccccc4)nc(-c4ccc(-n5c6ccccc6c6c5ccc5c7ccccc7n(-c7ccccc7)c56)cc4)n3)cc2)cc1] | 1154 |
| 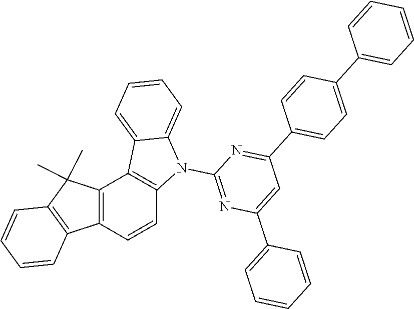 [SMILES: CC1(C)c2ccccc2-c2ccc3c(c21)c1ccccc1n3-c1nc(-c2ccccc2)cc(-c2ccc(-c3ccccc3)cc2)n1] | 1155 |
| 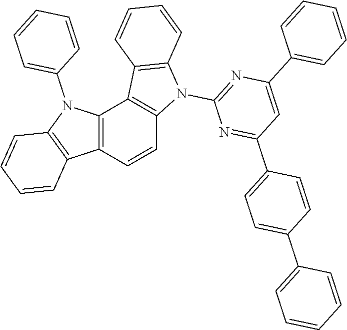 [SMILES: c1ccc(-c2ccc(-c3cc(-c4ccccc4)nc(-n4c5ccccc5c5c4ccc4c6ccccc6n(-c6ccccc6)c45)n3)cc2)cc1] | 1156 |
| 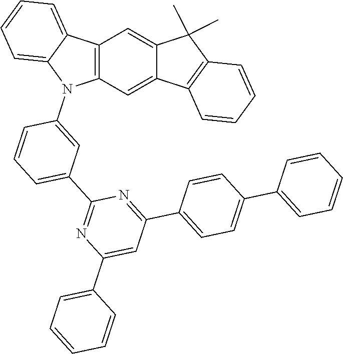 [SMILES: CC1(C)c2ccccc2-c2cc3c(cc21)c1ccccc1n3-c1cccc(-c2nc(-c3ccccc3)cc(-c3ccc(-c4ccccc4)cc3)n2)c1] | 1157 |
| 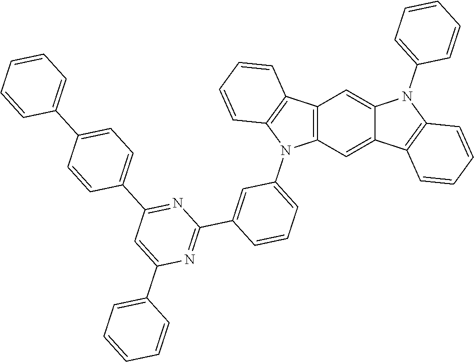 [SMILES: c1ccc(-c2ccc(-c3cc(-c4ccccc4)nc(-c4cccc(-n5c6ccccc6c6cc7c(cc65)c5ccccc5n7-c5ccccc5)c4)n3)cc2)cc1] | 1158 |
| 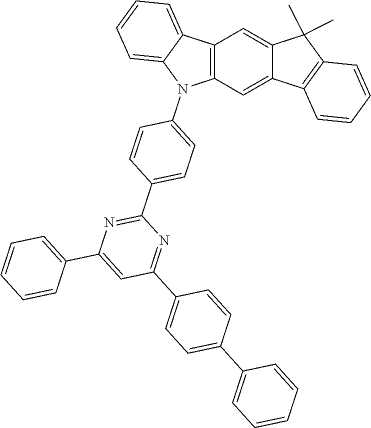 [SMILES: CC1(C)c2ccccc2-c2cc3c(cc21)c1ccccc1n3-c1ccc(-c2nc(-c3ccccc3)cc(-c3ccc(-c4ccccc4)cc3)n2)cc1] | 1159 |
| 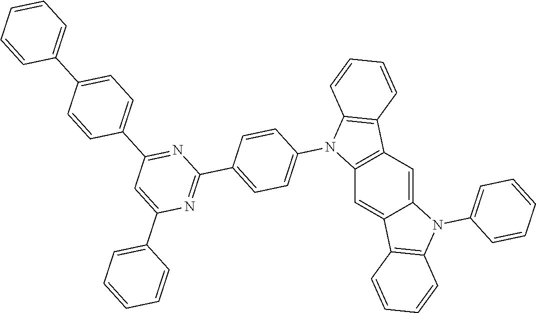 [SMILES: c1ccc(-c2ccc(-c3cc(-c4ccccc4)nc(-c4ccc(-n5c6ccccc6c6cc7c(cc65)c5ccccc5n7-c5ccccc5)cc4)n3)cc2)cc1] | 1160 |
| 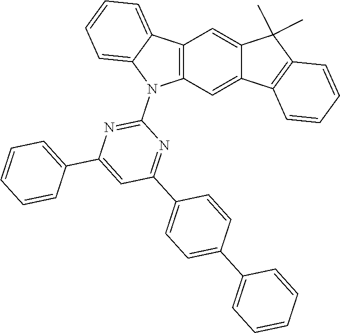 [SMILES: CC1(C)c2ccccc2-c2cc3c(cc21)c1ccccc1n3-c1nc(-c2ccccc2)cc(-c2ccc(-c3ccccc3)cc2)n1] | 1161 |
| 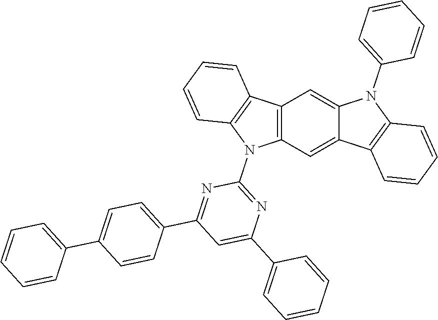 [SMILES: c1ccc(-c2ccc(-c3cc(-c4ccccc4)nc(-n4c5ccccc5c5cc6c(cc54)c4ccccc4n6-c4ccccc4)n3)cc2)cc1] | 1162 |
| 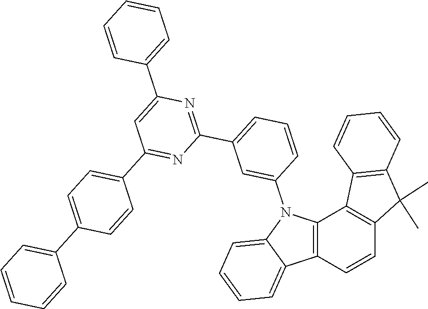 [SMILES: CC1(C)c2ccccc2-c2c1ccc1c3ccccc3n(-c3cccc(-c4nc(-c5ccccc5)cc(-c5ccc(-c6ccccc6)cc5)n4)c3)c21] | 1163 |
| 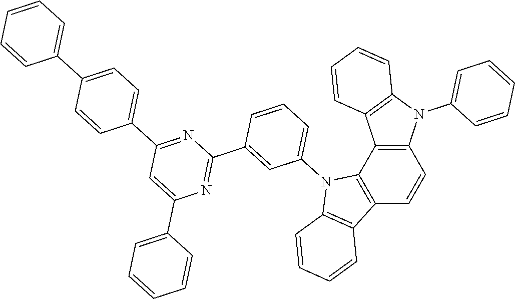 [SMILES: c1ccc(-c2ccc(-c3cc(-c4ccccc4)nc(-c4cccc(-n5c6ccccc6c6ccc7c(c8ccccc8n7-c7ccccc7)c65)c4)n3)cc2)cc1] | 1164 |
| 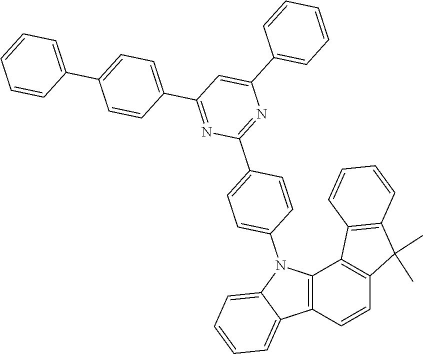 [SMILES: CC1(C)c2ccccc2-c2c1ccc1c3ccccc3n(-c3ccc(-c4nc(-c5ccccc5)cc(-c5ccc(-c6ccccc6)cc5)n4)cc3)c21] | 1165 |
| 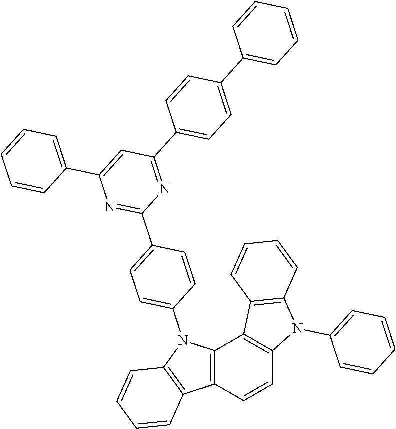 [SMILES: c1ccc(-c2ccc(-c3cc(-c4ccccc4)nc(-c4ccc(-n5c6ccccc6c6ccc7c(c8ccccc8n7-c7ccccc7)c65)cc4)n3)cc2)cc1] | 1166 |
| 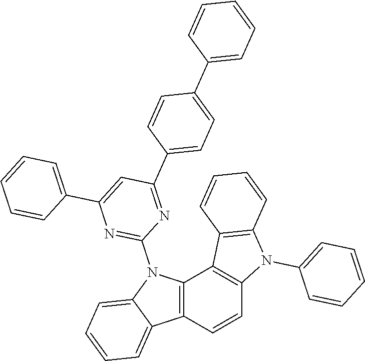 [SMILES: c1ccc(-c2ccc(-c3cc(-c4ccccc4)nc(-n4c5ccccc5c5ccc6c(c7ccccc7n6-c6ccccc6)c54)n3)cc2)cc1] | 1167 |
| 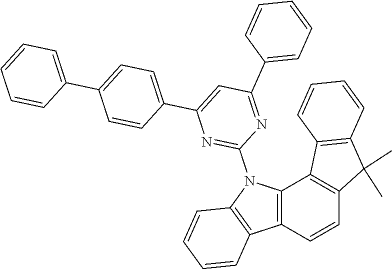 [SMILES: CC1(C)c2ccccc2-c2c1ccc1c3ccccc3n(-c3nc(-c4ccccc4)cc(-c4ccc(-c5ccccc5)cc4)n3)c21] | 1168 |
| 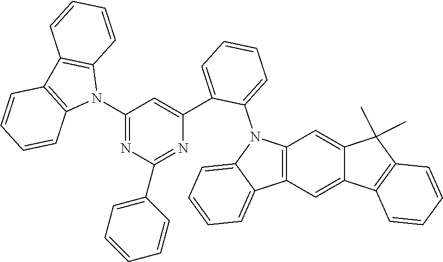 [SMILES: CC1(C)c2ccccc2-c2cc3c4ccccc4n(-c4ccccc4-c4cc(-n5c6ccccc6c6ccccc65)nc(-c5ccccc5)n4)c3cc21] | 1169 |
| 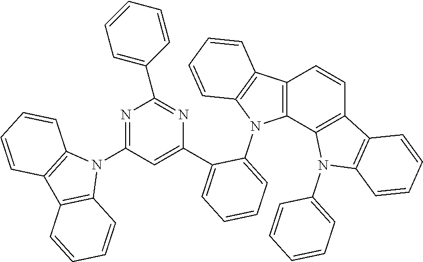 [SMILES: c1ccc(-c2nc(-c3ccccc3-n3c4ccccc4c4ccc5c6ccccc6n(-c6ccccc6)c5c43)cc(-n3c4ccccc4c4ccccc43)n2)cc1] | 1170 |
| 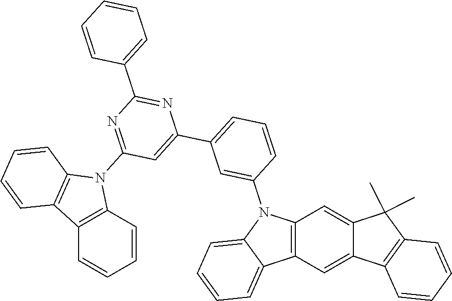 [SMILES: CC1(C)c2ccccc2-c2cc3c4ccccc4n(-c4cccc(-c5cc(-n6c7ccccc7c7ccccc76)nc(-c6ccccc6)n5)c4)c3cc21] | 1171 |
| 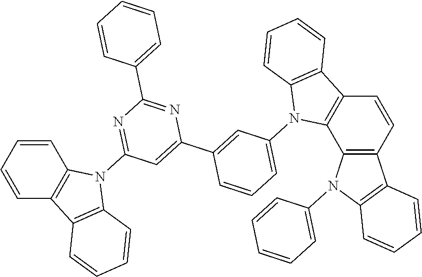 [SMILES: c1ccc(-c2nc(-c3cccc(-n4c5ccccc5c5ccc6c7ccccc7n(-c7ccccc7)c6c54)c3)cc(-n3c4ccccc4c4ccccc43)n2)cc1] | 1172 |
| 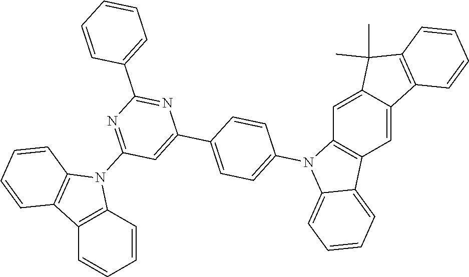 [SMILES: CC1(C)c2ccccc2-c2cc3c4ccccc4n(-c4ccc(-c5cc(-n6c7ccccc7c7ccccc76)nc(-c6ccccc6)n5)cc4)c3cc21] | 1173 |
| 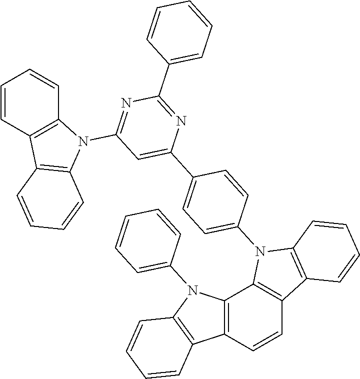 [SMILES: c1ccc(-c2nc(-c3ccc(-n4c5ccccc5c5ccc6c7ccccc7n(-c7ccccc7)c6c54)cc3)cc(-n3c4ccccc4c4ccccc43)n2)cc1] | 1174 |
| 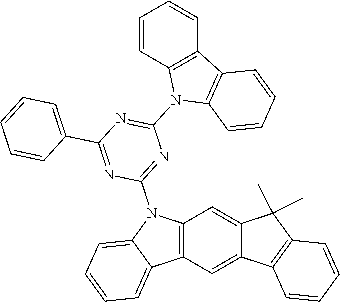 [SMILES: CC1(C)c2ccccc2-c2cc3c4ccccc4n(-c4nc(-c5ccccc5)nc(-n5c6ccccc6c6ccccc65)n4)c3cc21] | 1175 |
| 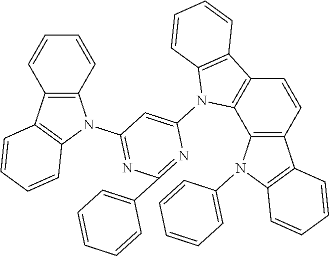 [SMILES: c1ccc(-c2nc(-n3c4ccccc4c4ccccc43)cc(-n3c4ccccc4c4ccc5c6ccccc6n(-c6ccccc6)c5c43)n2)cc1] | 1176 |
| 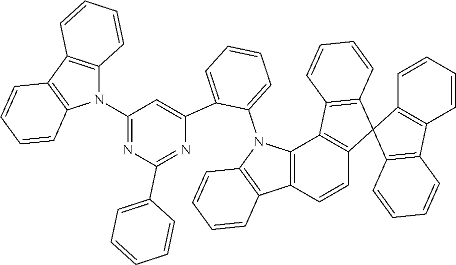 [SMILES: c1ccc(-c2nc(-c3ccccc3-n3c4ccccc4c4ccc5c(c43)-c3ccccc3C53c4ccccc4-c4ccccc43)cc(-n3c4ccccc4c4ccccc43)n2)cc1] | 1177 |
| 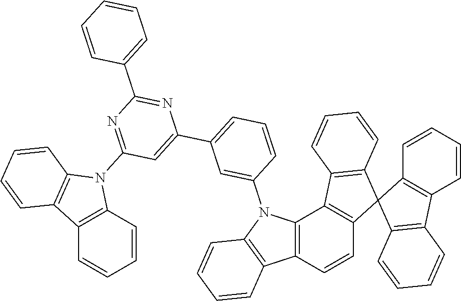 [SMILES: c1ccc(-c2nc(-c3cccc(-n4c5ccccc5c5ccc6c(c54)-c4ccccc4C64c5ccccc5-c5ccccc54)c3)cc(-n3c4ccccc4c4ccccc43)n2)cc1] | 1178 |
| 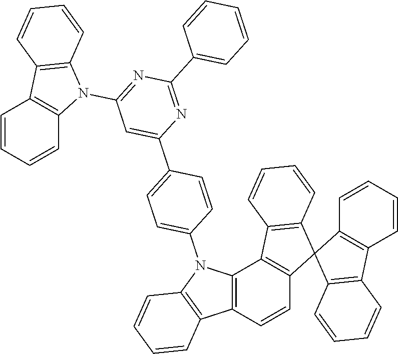 [SMILES: c1ccc(-c2nc(-c3ccc(-n4c5ccccc5c5ccc6c(c54)-c4ccccc4C64c5ccccc5-c5ccccc54)cc3)cc(-n3c4ccccc4c4ccccc43)n2)cc1] | 1179 |
| 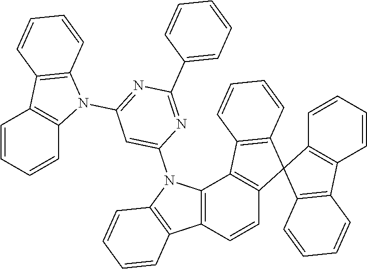 [SMILES: c1ccc(-c2nc(-n3c4ccccc4c4ccccc43)cc(-n3c4ccccc4c4ccc5c(c43)-c3ccccc3C53c4ccccc4-c4ccccc43)n2)cc1] | 1180 |
| 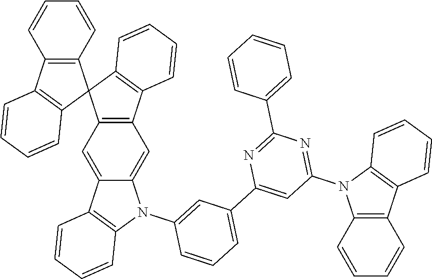 [SMILES: c1ccc(-c2nc(-c3cccc(-n4c5ccccc5c5cc6c(cc54)-c4ccccc4C64c5ccccc5-c5ccccc54)c3)cc(-n3c4ccccc4c4ccccc43)n2)cc1] | 1181 |
| 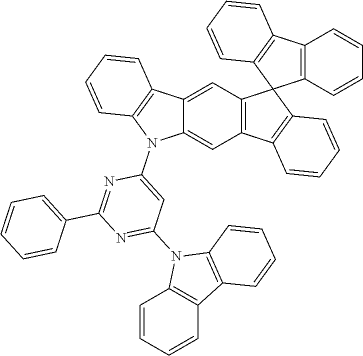 [SMILES: c1ccc(-c2nc(-n3c4ccccc4c4ccccc43)cc(-n3c4ccccc4c4cc5c(cc43)-c3ccccc3C53c4ccccc4-c4ccccc43)n2)cc1] | 1182 |
| 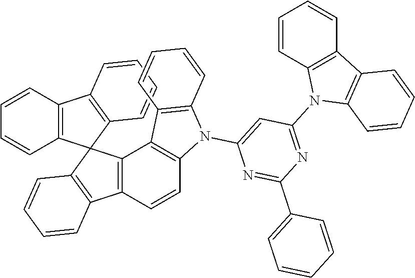 [SMILES: c1ccc(-c2nc(-n3c4ccccc4c4ccccc43)cc(-n3c4ccccc4c4c5c(ccc43)-c3ccccc3C53c4ccccc4-c4ccccc43)n2)cc1] | 1183 |
| 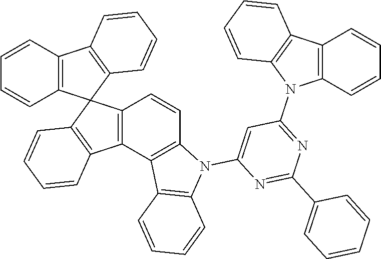 [SMILES: c1ccc(-c2nc(-n3c4ccccc4c4ccccc43)cc(-n3c4ccccc4c4c5c(ccc43)C3(c4ccccc4-c4ccccc43)c3ccccc3-5)n2)cc1] | 1184 |
| 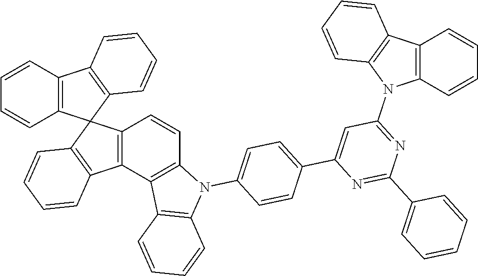 [SMILES: c1ccc(-c2nc(-c3ccc(-n4c5ccccc5c5c6c(ccc54)C4(c5ccccc5-c5ccccc54)c4ccccc4-6)cc3)cc(-n3c4ccccc4c4ccccc43)n2)cc1] | 1185 |
| 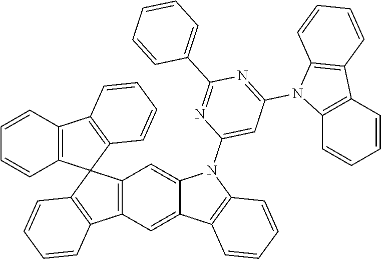 [SMILES: c1ccc(-c2nc(-n3c4ccccc4c4ccccc43)cc(-n3c4ccccc4c4cc5c(cc43)C3(c4ccccc4-c4ccccc43)c3ccccc3-5)n2)cc1] | 1186 |
| 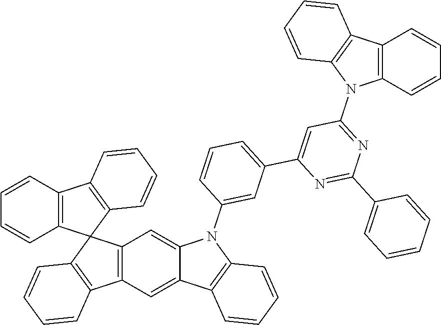 [SMILES: c1ccc(-c2nc(-c3cccc(-n4c5ccccc5c5cc6c(cc54)C4(c5ccccc5-c5ccccc54)c4ccccc4-6)c3)cc(-n3c4ccccc4c4ccccc43)n2)cc1] | 1187 |
| 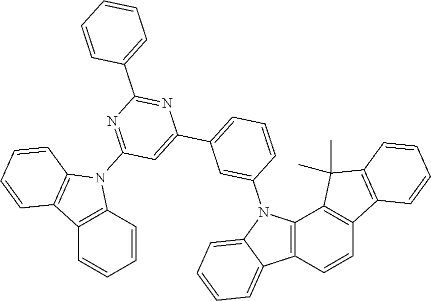 [SMILES: CC1(C)c2ccccc2-c2ccc3c4ccccc4n(-c4cccc(-c5cc(-n6c7ccccc7c7ccccc76)nc(-c6ccccc6)n5)c4)c3c21] | 1188 |
| 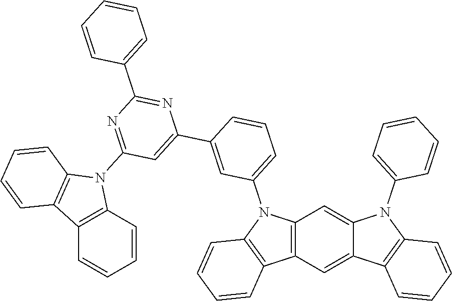 [SMILES: c1ccc(-c2nc(-c3cccc(-n4c5ccccc5c5cc6c7ccccc7n(-c7ccccc7)c6cc54)c3)cc(-n3c4ccccc4c4ccccc43)n2)cc1] | 1189 |
| 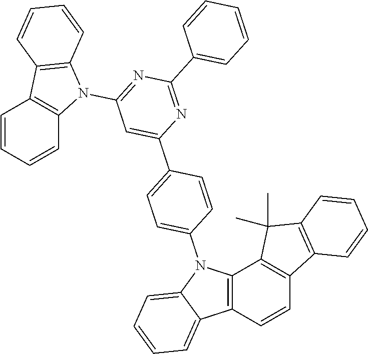 [SMILES: CC1(C)c2ccccc2-c2ccc3c4ccccc4n(-c4ccc(-c5cc(-n6c7ccccc7c7ccccc76)nc(-c6ccccc6)n5)cc4)c3c21] | 1190 |
| 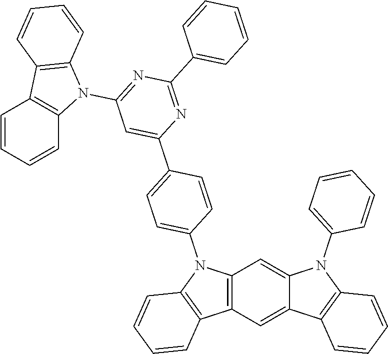 [SMILES: c1ccc(-c2nc(-c3ccc(-n4c5ccccc5c5cc6c7ccccc7n(-c7ccccc7)c6cc54)cc3)cc(-n3c4ccccc4c4ccccc43)n2)cc1] | 1191 |
| 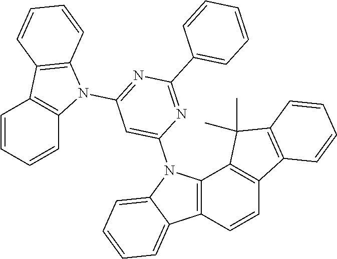 [SMILES: CC1(C)c2ccccc2-c2ccc3c4ccccc4n(-c4cc(-n5c6ccccc6c6ccccc65)nc(-c5ccccc5)n4)c3c21] | 1192 |
| 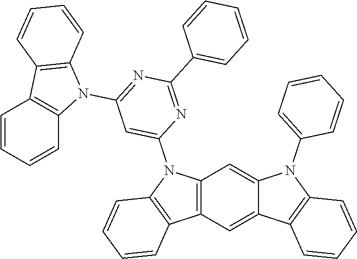 [SMILES: c1ccc(-c2nc(-n3c4ccccc4c4ccccc43)cc(-n3c4ccccc4c4cc5c6ccccc6n(-c6ccccc6)c5cc43)n2)cc1] | 1193 |
| 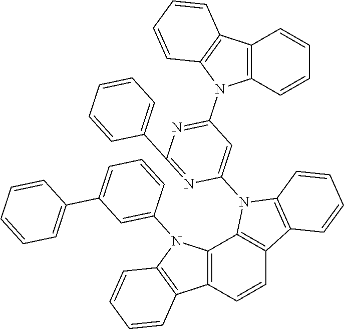 [SMILES: c1ccc(-c2cccc(-n3c4ccccc4c4ccc5c6ccccc6n(-c6cc(-n7c8ccccc8c8ccccc87)nc(-c7ccccc7)n6)c5c43)c2)cc1] | 1194 |
| 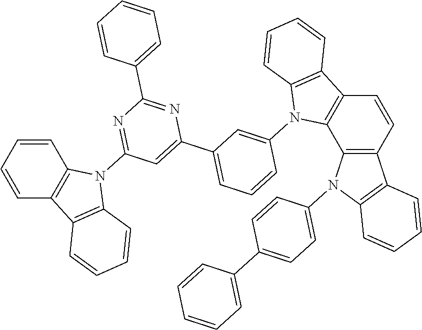 [SMILES: c1ccc(-c2ccc(-n3c4ccccc4c4ccc5c6ccccc6n(-c6cccc(-c7cc(-n8c9ccccc9c9ccccc98)nc(-c8ccccc8)n7)c6)c5c43)cc2)cc1] | 1195 |
| 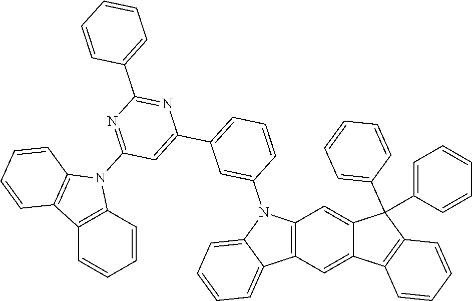 [SMILES: c1ccc(-c2nc(-c3cccc(-n4c5ccccc5c5cc6c(cc54)C(c4ccccc4)(c4ccccc4)c4ccccc4-6)c3)cc(-n3c4ccccc4c4ccccc43)n2)cc1] | 1196 |
| 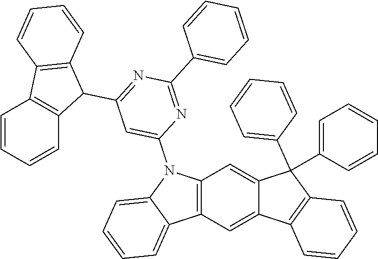 [SMILES: c1ccc(-c2nc(C3c4ccccc4-c4ccccc43)cc(-n3c4ccccc4c4cc5c(cc43)C(c3ccccc3)(c3ccccc3)c3ccccc3-5)n2)cc1] | 1197 |
| 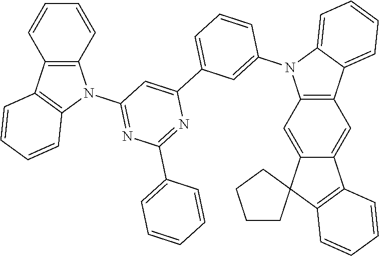 [SMILES: c1ccc(-c2nc(-c3cccc(-n4c5ccccc5c5cc6c(cc54)C4(CCCC4)c4ccccc4-6)c3)cc(-n3c4ccccc4c4ccccc43)n2)cc1] | 1198 |
| 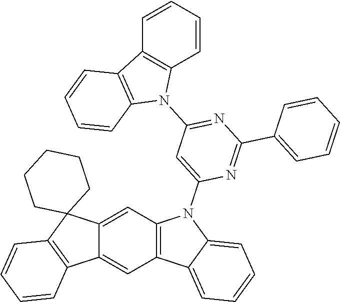 [SMILES: c1ccc(-c2nc(-n3c4ccccc4c4ccccc43)cc(-n3c4ccccc4c4cc5c(cc43)C3(CCCCC3)c3ccccc3-5)n2)cc1] | 1199 |
| 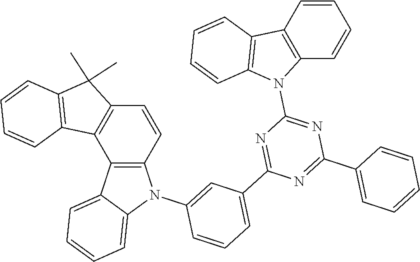 [SMILES: CC1(C)c2ccccc2-c2c1ccc1c2c2ccccc2n1-c1cccc(-c2nc(-c3ccccc3)nc(-n3c4ccccc4c4ccccc43)n2)c1] | 1200 |
| 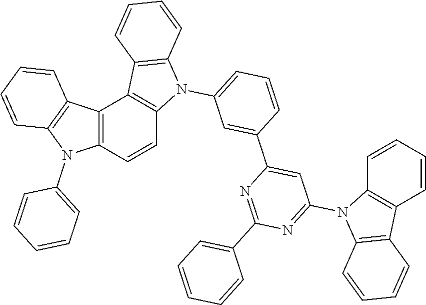 [SMILES: c1ccc(-c2nc(-c3cccc(-n4c5ccccc5c5c6c7ccccc7n(-c7ccccc7)c6ccc54)c3)cc(-n3c4ccccc4c4ccccc43)n2)cc1] | 1201 |
| 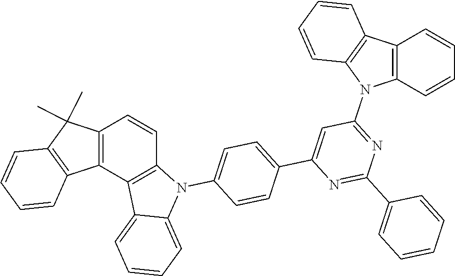 [SMILES: CC1(C)c2ccccc2-c2c1ccc1c2c2ccccc2n1-c1ccc(-c2cc(-n3c4ccccc4c4ccccc43)nc(-c3ccccc3)n2)cc1] | 1202 |
| 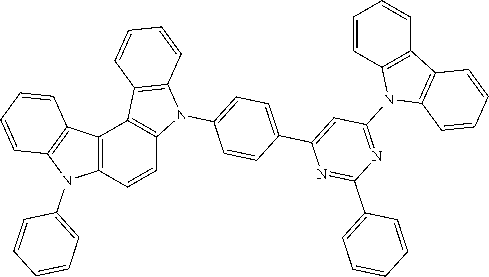 [SMILES: c1ccc(-c2nc(-c3ccc(-n4c5ccccc5c5c6c7ccccc7n(-c7ccccc7)c6ccc54)cc3)cc(-n3c4ccccc4c4ccccc43)n2)cc1] | 1203 |
| 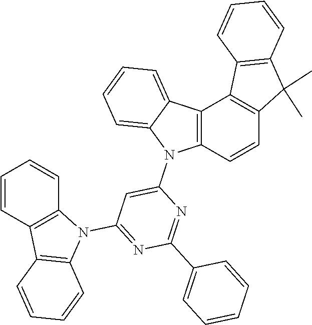 [SMILES: CC1(C)c2ccccc2-c2c1ccc1c2c2ccccc2n1-c1cc(-n2c3ccccc3c3ccccc32)nc(-c2ccccc2)n1] | 1204 |
| 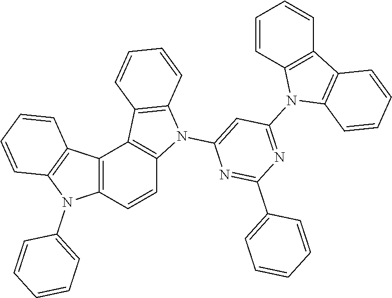 [SMILES: c1ccc(-c2nc(-n3c4ccccc4c4ccccc43)cc(-n3c4ccccc4c4c5c6ccccc6n(-c6ccccc6)c5ccc43)n2)cc1] | 1205 |
| 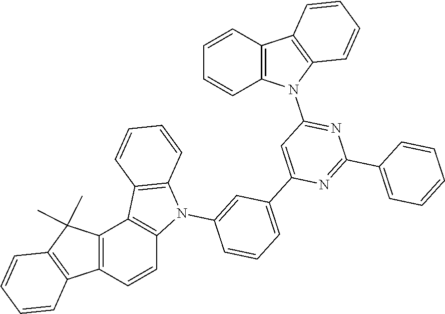 [SMILES: CC1(C)c2ccccc2-c2ccc3c(c21)c1ccccc1n3-c1cccc(-c2cc(-n3c4ccccc4c4ccccc43)nc(-c3ccccc3)n2)c1] | 1206 |
| 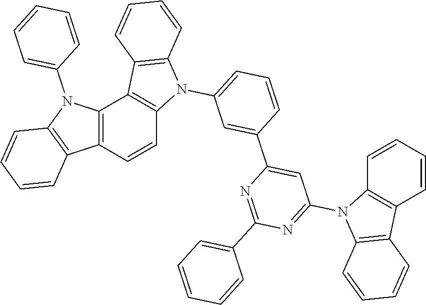 [SMILES: c1ccc(-c2nc(-c3cccc(-n4c5ccccc5c5c4ccc4c6ccccc6n(-c6ccccc6)c45)c3)cc(-n3c4ccccc4c4ccccc43)n2)cc1] | 1207 |
| 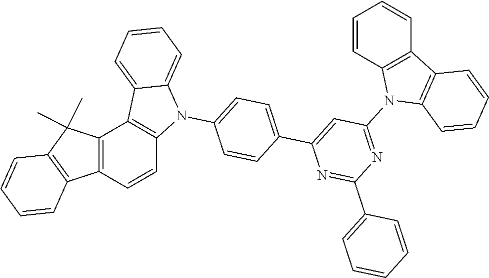 [SMILES: CC1(C)c2ccccc2-c2ccc3c(c21)c1ccccc1n3-c1ccc(-c2cc(-n3c4ccccc4c4ccccc43)nc(-c3ccccc3)n2)cc1] | 1208 |
| 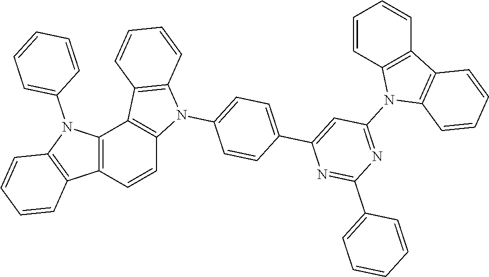 [SMILES: c1ccc(-c2nc(-c3ccc(-n4c5ccccc5c5c4ccc4c6ccccc6n(-c6ccccc6)c45)cc3)cc(-n3c4ccccc4c4ccccc43)n2)cc1] | 1209 |
| 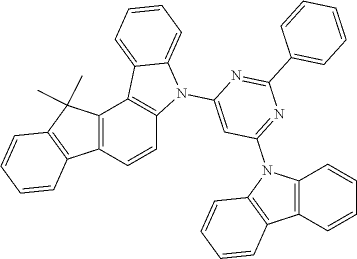 [SMILES: CC1(C)c2ccccc2-c2ccc3c(c21)c1ccccc1n3-c1cc(-n2c3ccccc3c3ccccc32)nc(-c2ccccc2)n1] | 1210 |
| 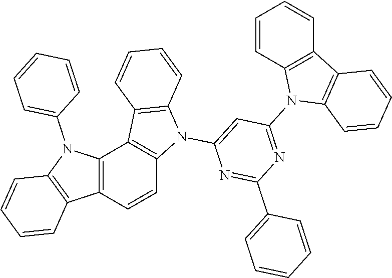 [SMILES: c1ccc(-c2nc(-n3c4ccccc4c4ccccc43)cc(-n3c4ccccc4c4c3ccc3c5ccccc5n(-c5ccccc5)c34)n2)cc1] | 1211 |
| 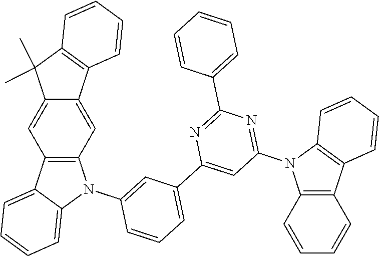 [SMILES: CC1(C)c2ccccc2-c2cc3c(cc21)c1ccccc1n3-c1cccc(-c2cc(-n3c4ccccc4c4ccccc43)nc(-c3ccccc3)n2)c1] | 1212 |
| 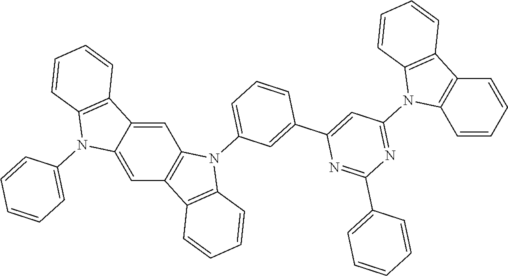 [SMILES: c1ccc(-c2nc(-c3cccc(-n4c5ccccc5c5cc6c(cc54)c4ccccc4n6-c4ccccc4)c3)cc(-n3c4ccccc4c4ccccc43)n2)cc1] | 1213 |
| 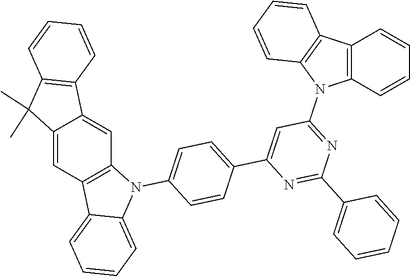 [SMILES: CC1(C)c2ccccc2-c2cc3c(cc21)c1ccccc1n3-c1ccc(-c2cc(-n3c4ccccc4c4ccccc43)nc(-c3ccccc3)n2)cc1] | 1214 |
| 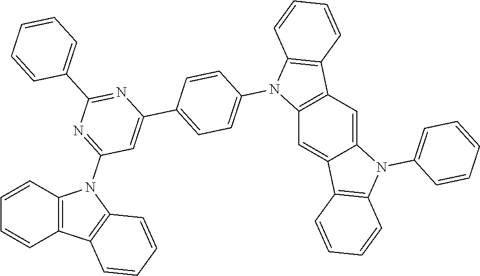 [SMILES: c1ccc(-c2nc(-c3ccc(-n4c5ccccc5c5cc6c(cc54)c4ccccc4n6-c4ccccc4)cc3)cc(-n3c4ccccc4c4ccccc43)n2)cc1] | 1215 |
| 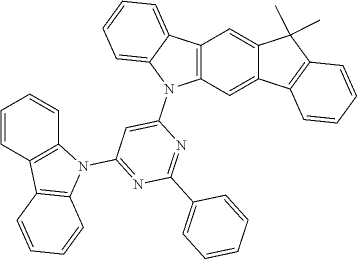 [SMILES: CC1(C)c2ccccc2-c2cc3c(cc21)c1ccccc1n3-c1cc(-n2c3ccccc3c3ccccc32)nc(-c2ccccc2)n1] | 1216 |
| 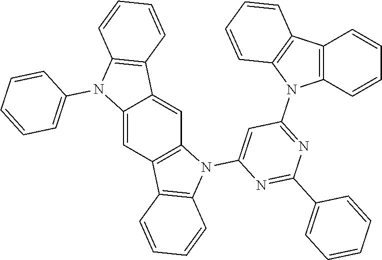 [SMILES: c1ccc(-c2nc(-n3c4ccccc4c4ccccc43)cc(-n3c4ccccc4c4cc5c(cc43)c3ccccc3n5-c3ccccc3)n2)cc1] | 1217 |
| 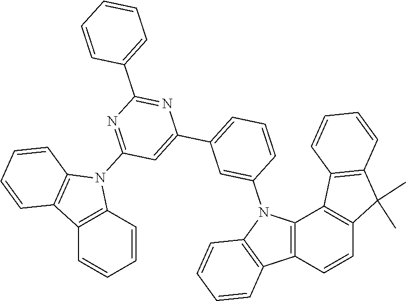 [SMILES: CC1(C)c2ccccc2-c2c1ccc1c3ccccc3n(-c3cccc(-c4cc(-n5c6ccccc6c6ccccc65)nc(-c5ccccc5)n4)c3)c21] | 1218 |
| 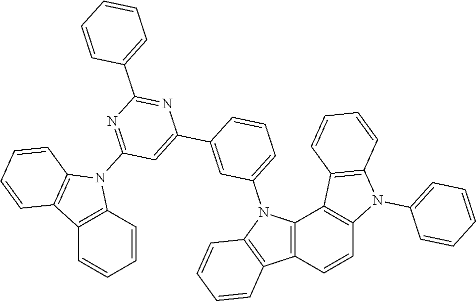 [SMILES: c1ccc(-c2nc(-c3cccc(-n4c5ccccc5c5ccc6c(c7ccccc7n6-c6ccccc6)c54)c3)cc(-n3c4ccccc4c4ccccc43)n2)cc1] | 1219 |
| 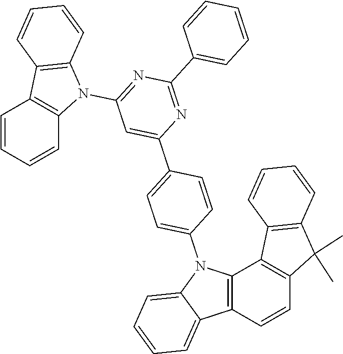 [SMILES: CC1(C)c2ccccc2-c2c1ccc1c3ccccc3n(-c3ccc(-c4cc(-n5c6ccccc6c6ccccc65)nc(-c5ccccc5)n4)cc3)c21] | 1220 |
| 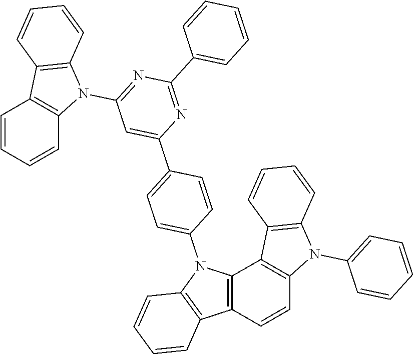 [SMILES: c1ccc(-c2nc(-c3ccc(-n4c5ccccc5c5ccc6c(c7ccccc7n6-c6ccccc6)c54)cc3)cc(-n3c4ccccc4c4ccccc43)n2)cc1] | 1221 |
| 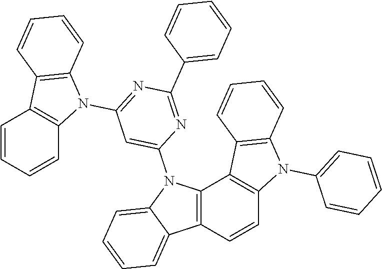 [SMILES: c1ccc(-c2nc(-n3c4ccccc4c4ccccc43)cc(-n3c4ccccc4c4ccc5c(c6ccccc6n5-c5ccccc5)c43)n2)cc1] | 1222 |
| 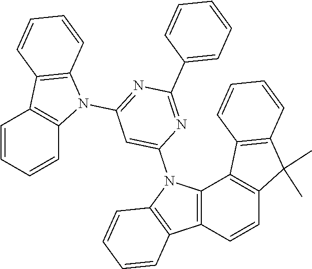 [SMILES: CC1(C)c2ccccc2-c2c1ccc1c3ccccc3n(-c3cc(-n4c5ccccc5c5ccccc54)nc(-c4ccccc4)n3)c21] | 1223 |
| 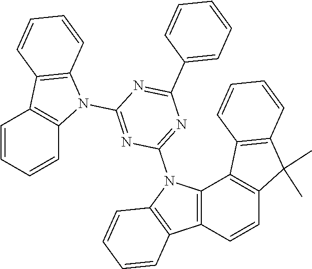 [SMILES: CC1(C)c2ccccc2-c2c1ccc1c3ccccc3n(-c3nc(-c4ccccc4)nc(-n4c5ccccc5c5ccccc54)n3)c21] | 1224 |
| 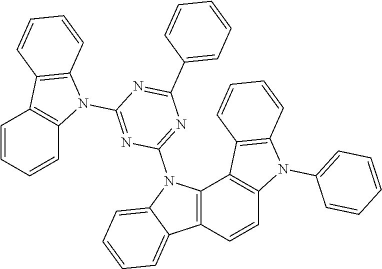 [SMILES: c1ccc(-c2nc(-n3c4ccccc4c4ccccc43)nc(-n3c4ccccc4c4ccc5c(c6ccccc6n5-c5ccccc5)c43)n2)cc1] | 1225 |
| 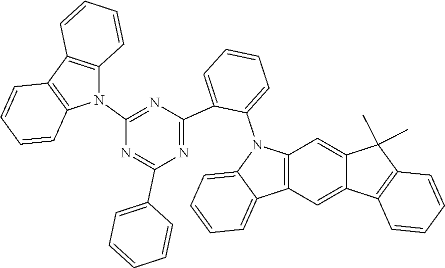 [SMILES: CC1(C)c2ccccc2-c2cc3c4ccccc4n(-c4ccccc4-c4nc(-c5ccccc5)nc(-n5c6ccccc6c6ccccc65)n4)c3cc21] | 1226 |
| 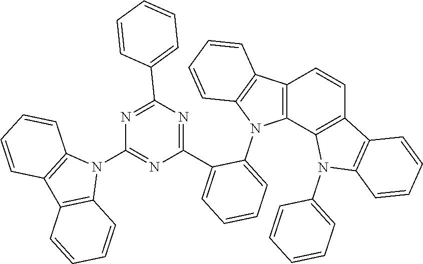 [SMILES: c1ccc(-c2nc(-c3ccccc3-n3c4ccccc4c4ccc5c6ccccc6n(-c6ccccc6)c5c43)nc(-n3c4ccccc4c4ccccc43)n2)cc1] | 1227 |
| 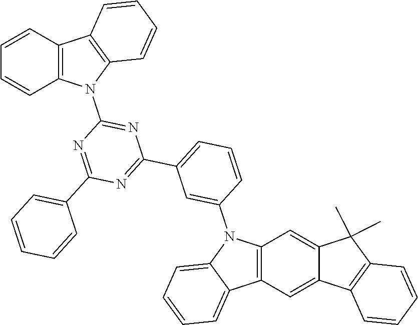 [SMILES: CC1(C)c2ccccc2-c2cc3c4ccccc4n(-c4cccc(-c5nc(-c6ccccc6)nc(-n6c7ccccc7c7ccccc76)n5)c4)c3cc21] | 1228 |
| 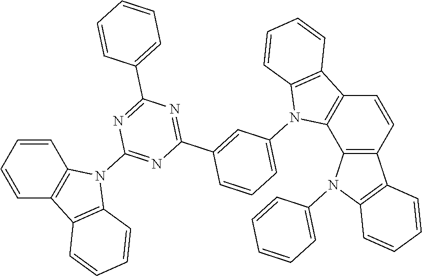 [SMILES: c1ccc(-c2nc(-c3cccc(-n4c5ccccc5c5ccc6c7ccccc7n(-c7ccccc7)c6c54)c3)nc(-n3c4ccccc4c4ccccc43)n2)cc1] | 1229 |
| 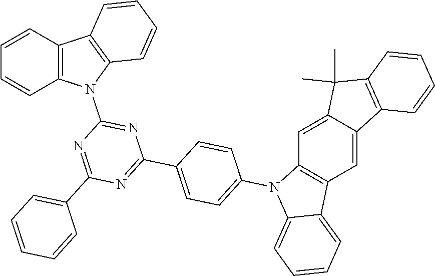 [SMILES: CC1(C)c2ccccc2-c2cc3c4ccccc4n(-c4ccc(-c5nc(-c6ccccc6)nc(-n6c7ccccc7c7ccccc76)n5)cc4)c3cc21] | 1230 |
| 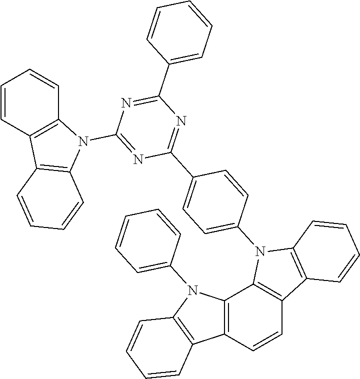 [SMILES: c1ccc(-c2nc(-c3ccc(-n4c5ccccc5c5ccc6c7ccccc7n(-c7ccccc7)c6c54)cc3)nc(-n3c4ccccc4c4ccccc43)n2)cc1] | 1231 |
| 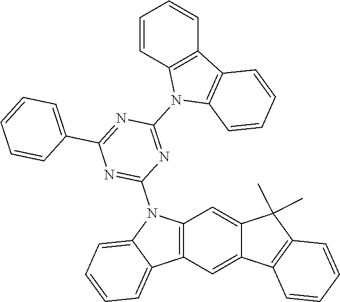 [SMILES: CC1(C)c2ccccc2-c2cc3c4ccccc4n(-c4nc(-c5ccccc5)nc(-n5c6ccccc6c6ccccc65)n4)c3cc21] | 1232 |
| 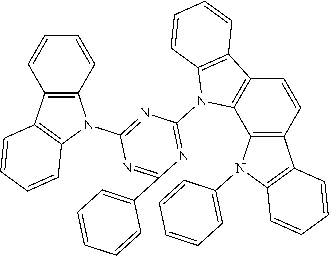 [SMILES: c1ccc(-c2nc(-n3c4ccccc4c4ccccc43)nc(-n3c4ccccc4c4ccc5c6ccccc6n(-c6ccccc6)c5c43)n2)cc1] | 1233 |
| 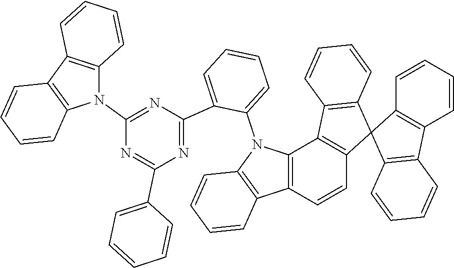 [SMILES: c1ccc(-c2nc(-c3ccccc3-n3c4ccccc4c4ccc5c(c43)-c3ccccc3C53c4ccccc4-c4ccccc43)nc(-n3c4ccccc4c4ccccc43)n2)cc1] | 1234 |
| 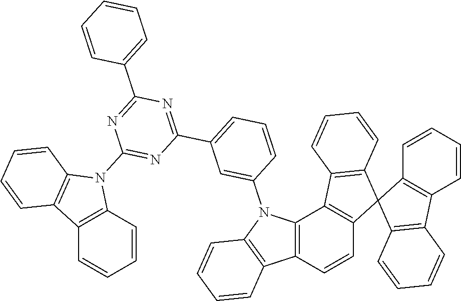 [SMILES: c1ccc(-c2nc(-c3cccc(-n4c5ccccc5c5ccc6c(c54)-c4ccccc4C64c5ccccc5-c5ccccc54)c3)nc(-n3c4ccccc4c4ccccc43)n2)cc1] | 1235 |
| 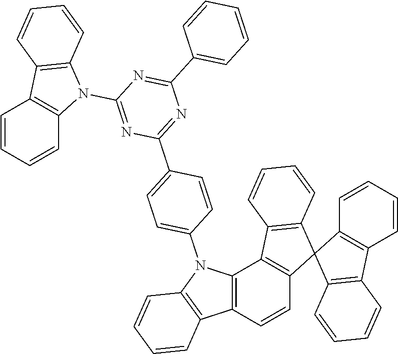 [SMILES: c1ccc(-c2nc(-c3ccc(-n4c5ccccc5c5ccc6c(c54)-c4ccccc4C64c5ccccc5-c5ccccc54)cc3)nc(-n3c4ccccc4c4ccccc43)n2)cc1] | 1236 |
| 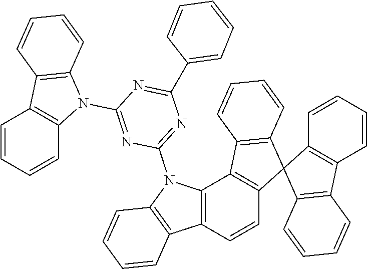 [SMILES: c1ccc(-c2nc(-n3c4ccccc4c4ccccc43)nc(-n3c4ccccc4c4ccc5c(c43)-c3ccccc3C53c4ccccc4-c4ccccc43)n2)cc1] | 1237 |
| 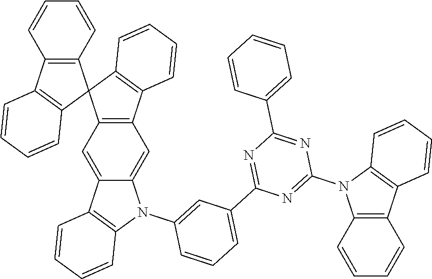 [SMILES: c1ccc(-c2nc(-c3cccc(-n4c5ccccc5c5cc6c(cc54)-c4ccccc4C64c5ccccc5-c5ccccc54)c3)nc(-n3c4ccccc4c4ccccc43)n2)cc1] | 1238 |
| 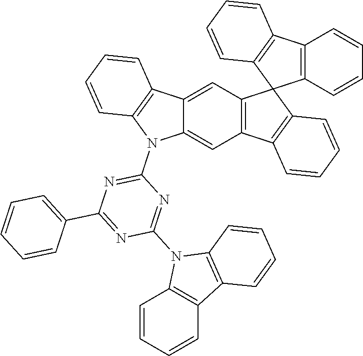 [SMILES: c1ccc(-c2nc(-n3c4ccccc4c4ccccc43)nc(-n3c4ccccc4c4cc5c(cc43)-c3ccccc3C53c4ccccc4-c4ccccc43)n2)cc1] | 1239 |
| 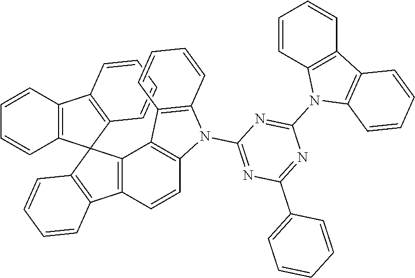 [SMILES: c1ccc(-c2nc(-n3c4ccccc4c4ccccc43)nc(-n3c4ccccc4c4c5c(ccc43)-c3ccccc3C53c4ccccc4-c4ccccc43)n2)cc1] | 1240 |
| 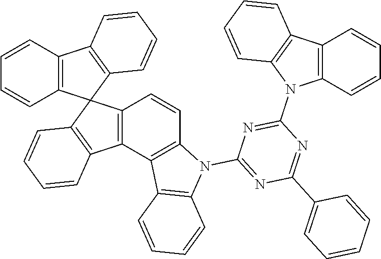 [SMILES: c1ccc(-c2nc(-n3c4ccccc4c4ccccc43)nc(-n3c4ccccc4c4c5c(ccc43)C3(c4ccccc4-c4ccccc43)c3ccccc3-5)n2)cc1] | 1241 |
| 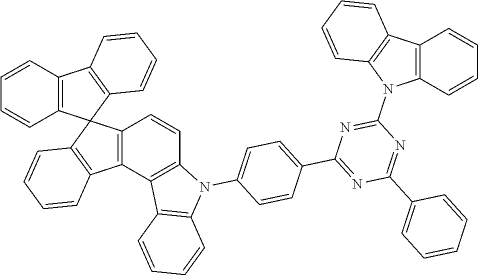 [SMILES: c1ccc(-c2nc(-c3ccc(-n4c5ccccc5c5c6c(ccc54)C4(c5ccccc5-c5ccccc54)c4ccccc4-6)cc3)nc(-n3c4ccccc4c4ccccc43)n2)cc1] | 1242 |
| 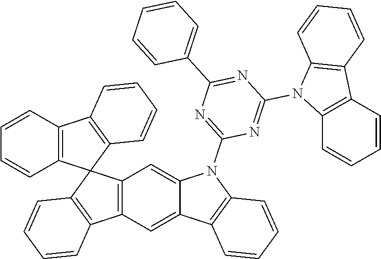 [SMILES: c1ccc(-c2nc(-n3c4ccccc4c4ccccc43)nc(-n3c4ccccc4c4cc5c(cc43)C3(c4ccccc4-c4ccccc43)c3ccccc3-5)n2)cc1] | 1243 |
| 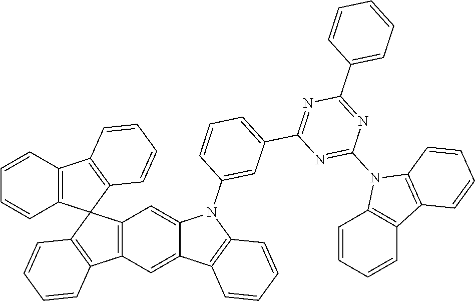 [SMILES: c1ccc(-c2nc(-c3cccc(-n4c5ccccc5c5cc6c(cc54)C4(c5ccccc5-c5ccccc54)c4ccccc4-6)c3)nc(-n3c4ccccc4c4ccccc43)n2)cc1] | 1244 |
| 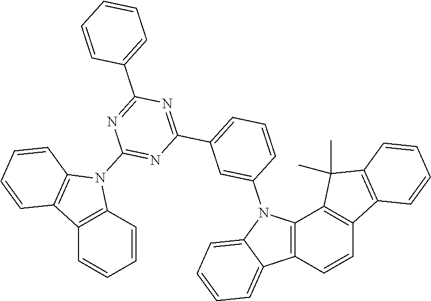 [SMILES: CC1(C)c2ccccc2-c2ccc3c4ccccc4n(-c4cccc(-c5nc(-c6ccccc6)nc(-n6c7ccccc7c7ccccc76)n5)c4)c3c21] | 1245 |
| 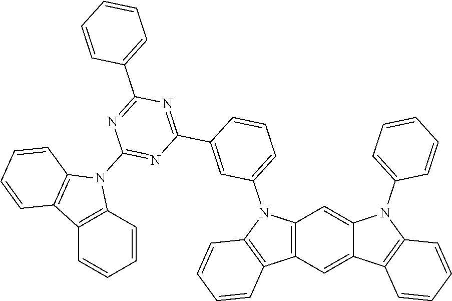 [SMILES: c1ccc(-c2nc(-c3cccc(-n4c5ccccc5c5cc6c7ccccc7n(-c7ccccc7)c6cc54)c3)nc(-n3c4ccccc4c4ccccc43)n2)cc1] | 1246 |
| 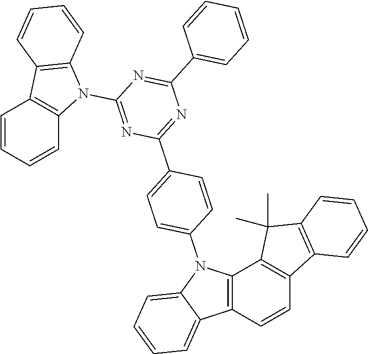 [SMILES: CC1(C)c2ccccc2-c2ccc3c4ccccc4n(-c4ccc(-c5nc(-c6ccccc6)nc(-n6c7ccccc7c7ccccc76)n5)cc4)c3c21] | 1247 |
| 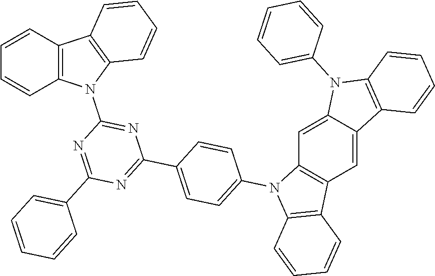 [SMILES: c1ccc(-c2nc(-c3ccc(-n4c5ccccc5c5cc6c7ccccc7n(-c7ccccc7)c6cc54)cc3)nc(-n3c4ccccc4c4ccccc43)n2)cc1] | 1248 |
| 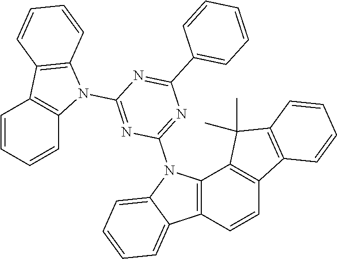 [SMILES: CC1(C)c2ccccc2-c2ccc3c4ccccc4n(-c4nc(-c5ccccc5)nc(-n5c6ccccc6c6ccccc65)n4)c3c21] | 1249 |
| 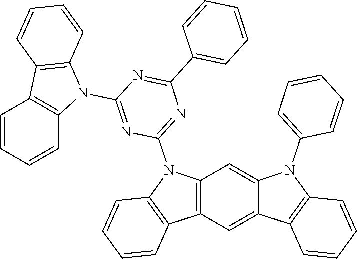 [SMILES: c1ccc(-c2nc(-n3c4ccccc4c4ccccc43)nc(-n3c4ccccc4c4cc5c6ccccc6n(-c6ccccc6)c5cc43)n2)cc1] | 1250 |
| 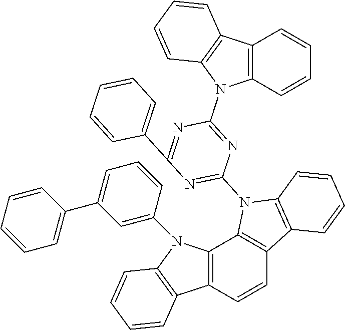 [SMILES: c1ccc(-c2cccc(-n3c4ccccc4c4ccc5c6ccccc6n(-c6nc(-c7ccccc7)nc(-n7c8ccccc8c8ccccc87)n6)c5c43)c2)cc1] | 1251 |
| 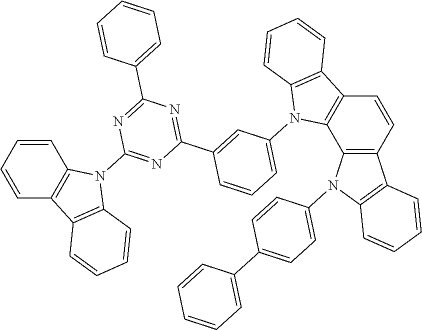 [SMILES: c1ccc(-c2ccc(-n3c4ccccc4c4ccc5c6ccccc6n(-c6cccc(-c7nc(-c8ccccc8)nc(-n8c9ccccc9c9ccccc98)n7)c6)c5c43)cc2)cc1] | 1252 |
| 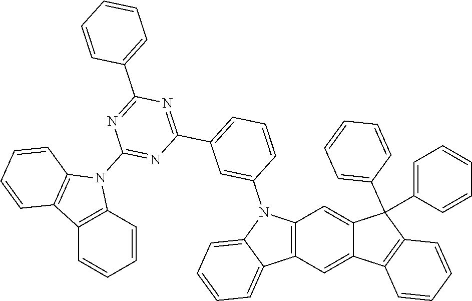 [SMILES: c1ccc(-c2nc(-c3cccc(-n4c5ccccc5c5cc6c(cc54)C(c4ccccc4)(c4ccccc4)c4ccccc4-6)c3)nc(-n3c4ccccc4c4ccccc43)n2)cc1] | 1253 |
| 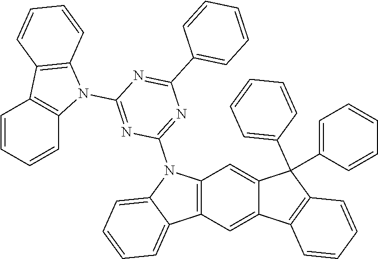 [SMILES: c1ccc(-c2nc(-n3c4ccccc4c4ccccc43)nc(-n3c4ccccc4c4cc5c(cc43)C(c3ccccc3)(c3ccccc3)c3ccccc3-5)n2)cc1] | 1254 |
| 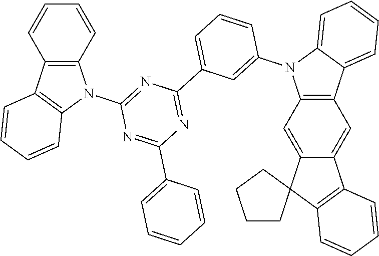 [SMILES: c1ccc(-c2nc(-c3cccc(-n4c5ccccc5c5cc6c(cc54)C4(CCCC4)c4ccccc4-6)c3)nc(-n3c4ccccc4c4ccccc43)n2)cc1] | 1255 |
| 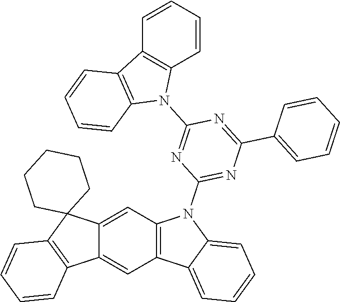 [SMILES: c1ccc(-c2nc(-n3c4ccccc4c4ccccc43)nc(-n3c4ccccc4c4cc5c(cc43)C3(CCCCC3)c3ccccc3-5)n2)cc1] | 1256 |
| 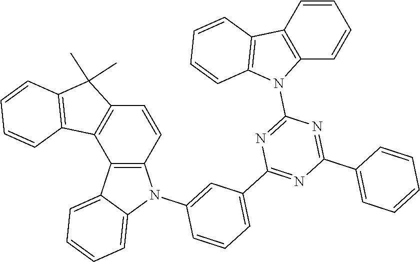 [SMILES: CC1(C)c2ccccc2-c2c1ccc1c2c2ccccc2n1-c1cccc(-c2nc(-c3ccccc3)nc(-n3c4ccccc4c4ccccc43)n2)c1] | 1257 |
| 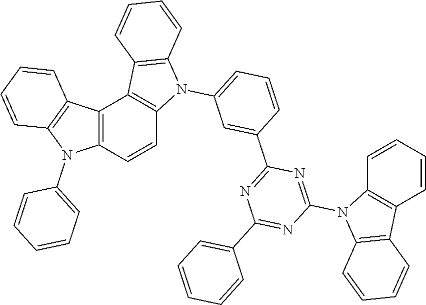 [SMILES: c1ccc(-c2nc(-c3cccc(-n4c5ccccc5c5c6c7ccccc7n(-c7ccccc7)c6ccc54)c3)nc(-n3c4ccccc4c4ccccc43)n2)cc1] | 1258 |
| 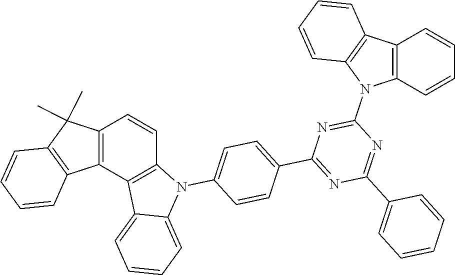 [SMILES: CC1(C)c2ccccc2-c2c1ccc1c2c2ccccc2n1-c1ccc(-c2nc(-c3ccccc3)nc(-n3c4ccccc4c4ccccc43)n2)cc1] | 1259 |
| 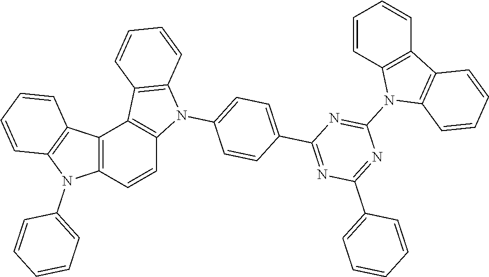 [SMILES: c1ccc(-c2nc(-c3ccc(-n4c5ccccc5c5c6c7ccccc7n(-c7ccccc7)c6ccc54)cc3)nc(-n3c4ccccc4c4ccccc43)n2)cc1] | 1260 |
| 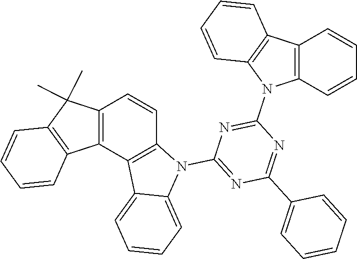 [SMILES: CC1(C)c2ccccc2-c2c1ccc1c2c2ccccc2n1-c1nc(-c2ccccc2)nc(-n2c3ccccc3c3ccccc32)n1] | 1261 |
| 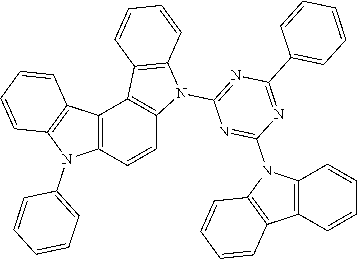 [SMILES: c1ccc(-c2nc(-n3c4ccccc4c4ccccc43)nc(-n3c4ccccc4c4c5c6ccccc6n(-c6ccccc6)c5ccc43)n2)cc1] | 1262 |
| 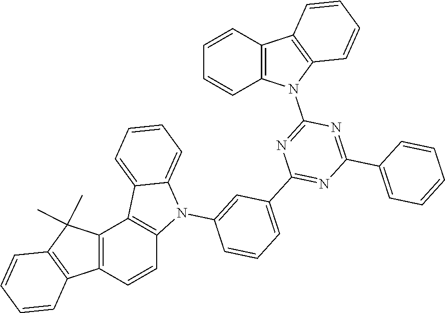 [SMILES: CC1(C)c2ccccc2-c2ccc3c(c21)c1ccccc1n3-c1cccc(-c2nc(-c3ccccc3)nc(-n3c4ccccc4c4ccccc43)n2)c1] | 1263 |
| 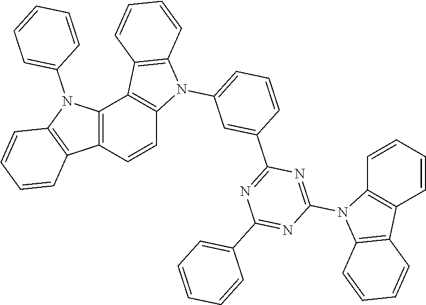 [SMILES: c1ccc(-c2nc(-c3cccc(-n4c5ccccc5c5c4ccc4c6ccccc6n(-c6ccccc6)c45)c3)nc(-n3c4ccccc4c4ccccc43)n2)cc1] | 1264 |
| 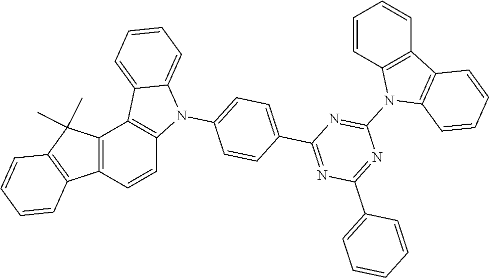 [SMILES: CC1(C)c2ccccc2-c2ccc3c(c21)c1ccccc1n3-c1ccc(-c2nc(-c3ccccc3)nc(-n3c4ccccc4c4ccccc43)n2)cc1] | 1265 |
| 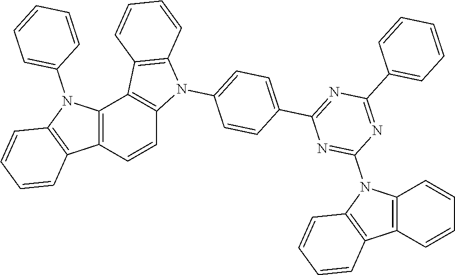 [SMILES: c1ccc(-c2nc(-c3ccc(-n4c5ccccc5c5c4ccc4c6ccccc6n(-c6ccccc6)c45)cc3)nc(-n3c4ccccc4c4ccccc43)n2)cc1] | 1266 |
| 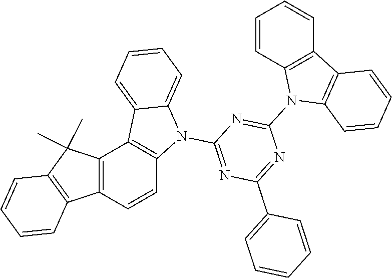 [SMILES: CC1(C)c2ccccc2-c2ccc3c(c21)c1ccccc1n3-c1nc(-c2ccccc2)nc(-n2c3ccccc3c3ccccc32)n1] | 1267 |
| 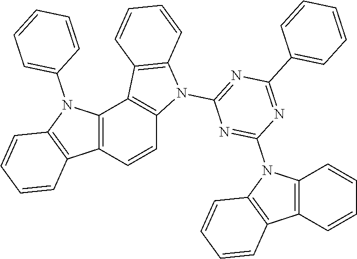 [SMILES: c1ccc(-c2nc(-n3c4ccccc4c4ccccc43)nc(-n3c4ccccc4c4c3ccc3c5ccccc5n(-c5ccccc5)c34)n2)cc1] | 1268 |
| 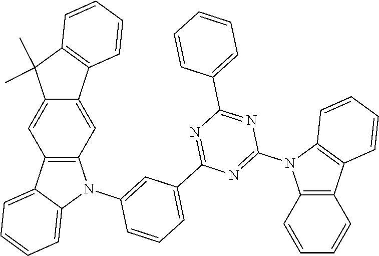 [SMILES: CC1(C)c2ccccc2-c2cc3c(cc21)c1ccccc1n3-c1cccc(-c2nc(-c3ccccc3)nc(-n3c4ccccc4c4ccccc43)n2)c1] | 1269 |
| 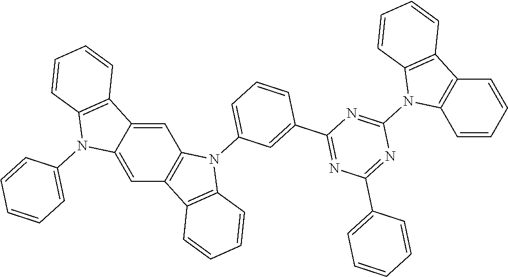 [SMILES: c1ccc(-c2nc(-c3cccc(-n4c5ccccc5c5cc6c(cc54)c4ccccc4n6-c4ccccc4)c3)nc(-n3c4ccccc4c4ccccc43)n2)cc1] | 1270 |
| 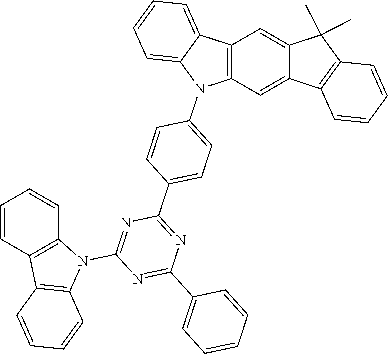 [SMILES: CC1(C)c2ccccc2-c2cc3c(cc21)c1ccccc1n3-c1ccc(-c2nc(-c3ccccc3)nc(-n3c4ccccc4c4ccccc43)n2)cc1] | 1271 |
| 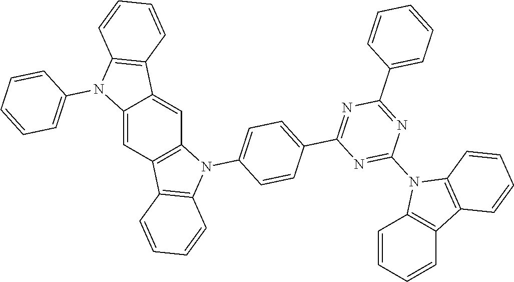 [SMILES: c1ccc(-c2nc(-c3ccc(-n4c5ccccc5c5cc6c(cc54)c4ccccc4n6-c4ccccc4)cc3)nc(-n3c4ccccc4c4ccccc43)n2)cc1] | 1272 |
| 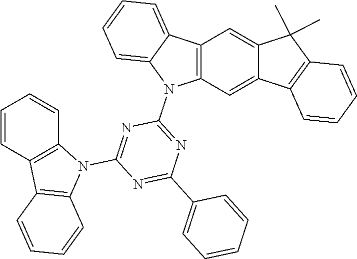 [SMILES: CC1(C)c2ccccc2-c2cc3c(cc21)c1ccccc1n3-c1nc(-c2ccccc2)nc(-n2c3ccccc3c3ccccc32)n1] | 1272 |
| 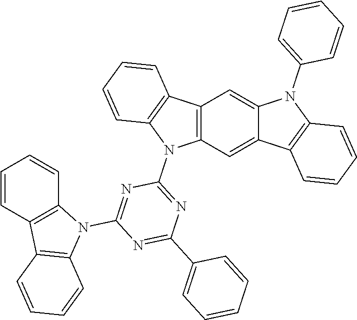 [SMILES: c1ccc(-c2nc(-n3c4ccccc4c4ccccc43)nc(-n3c4ccccc4c4cc5c(cc43)c3ccccc3n5-c3ccccc3)n2)cc1] | 1273 |
| 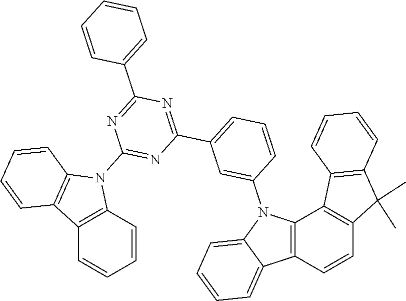 [SMILES: CC1(C)c2ccccc2-c2c1ccc1c3ccccc3n(-c3cccc(-c4nc(-c5ccccc5)nc(-n5c6ccccc6c6ccccc65)n4)c3)c21] | 1273 |
| 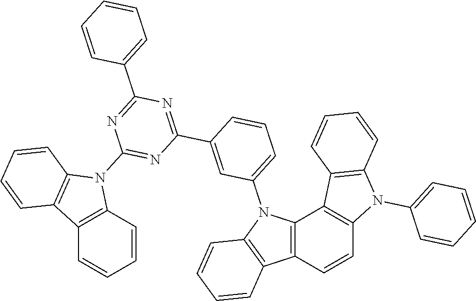 [SMILES: c1ccc(-c2nc(-c3cccc(-n4c5ccccc5c5ccc6c(c7ccccc7n6-c6ccccc6)c54)c3)nc(-n3c4ccccc4c4ccccc43)n2)cc1] | 1274 |
| 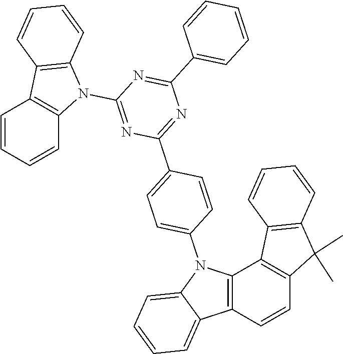 [SMILES: CC1(C)c2ccccc2-c2c1ccc1c3ccccc3n(-c3ccc(-c4nc(-c5ccccc5)nc(-n5c6ccccc6c6ccccc65)n4)cc3)c21] | 1275 |
| 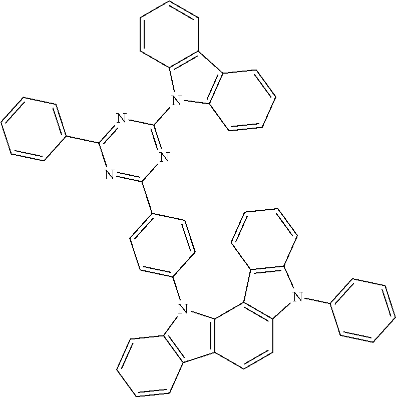 [SMILES: c1ccc(-c2nc(-c3ccc(-n4c5ccccc5c5ccc6c(c7ccccc7n6-c6ccccc6)c54)cc3)nc(-n3c4ccccc4c4ccccc43)n2)cc1] | 1276 |
| 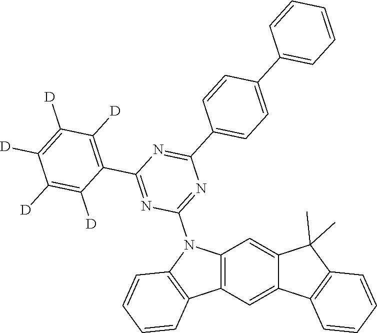 [SMILES: [2H]c1c([2H])c([2H])c(-c2nc(-c3ccc(-c4ccccc4)cc3)nc(-n3c4ccccc4c4cc5c(cc43)C(C)(C)c3ccccc3-5)n2)c([2H])c1[2H]] | 1277 |
| 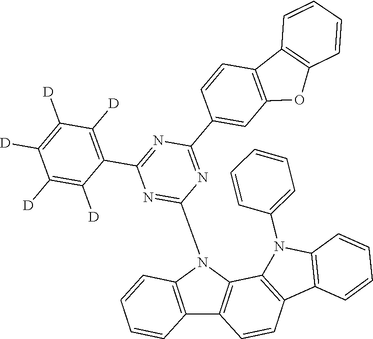 [SMILES: [2H]c1c([2H])c([2H])c(-c2nc(-c3ccc4c(c3)oc3ccccc34)nc(-n3c4ccccc4c4ccc5c6ccccc6n(-c6ccccc6)c5c43)n2)c([2H])c1[2H]] | 1278 |
| 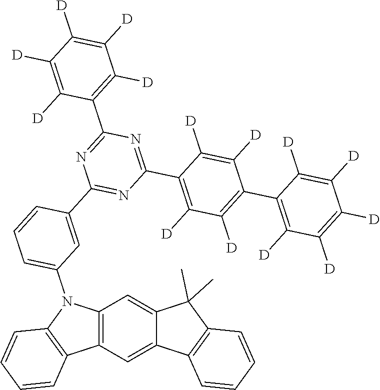 [SMILES: [2H]c1c([2H])c([2H])c(-c2nc(-c3cccc(-n4c5ccccc5c5cc6c(cc54)C(C)(C)c4ccccc4-6)c3)nc(-c3c([2H])c([2H])c(-c4c([2H])c([2H])c([2H])c([2H])c4[2H])c([2H])c3[2H])n2)c([2H])c1[2H]] | 1279 |
| 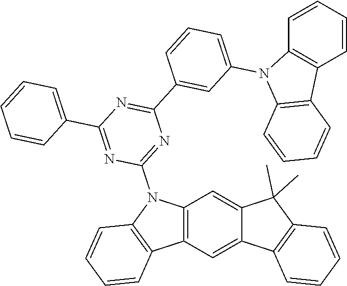 [SMILES: CC1(C)c2ccccc2-c2cc3c4ccccc4n(-c4nc(-c5ccccc5)nc(-c5cccc(-n6c7ccccc7c7ccccc76)c5)n4)c3cc21] | 1280 |
| 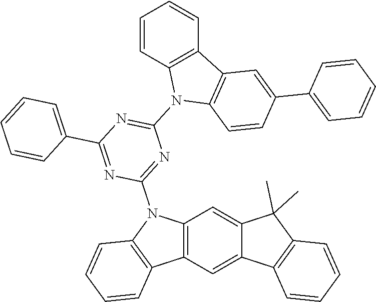 [SMILES: CC1(C)c2ccccc2-c2cc3c4ccccc4n(-c4nc(-c5ccccc5)nc(-n5c6ccccc6c6cc(-c7ccccc7)ccc65)n4)c3cc21] | 1281 |
| 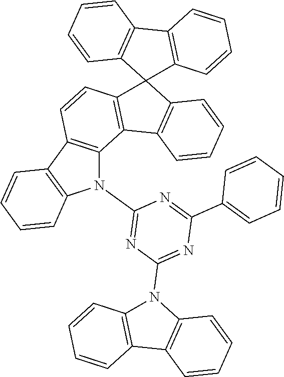 [SMILES: c1ccc(-c2nc(-n3c4ccccc4c4ccccc43)nc(-n3c4ccccc4c4ccc5c(c43)-c3ccccc3C53c4ccccc4-c4ccccc43)n2)cc1] | 1282 |
| 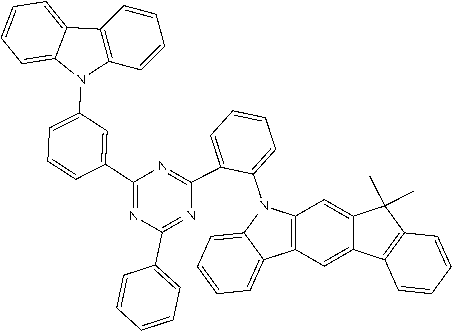 [SMILES: CC1(C)c2ccccc2-c2cc3c4ccccc4n(-c4ccccc4-c4nc(-c5ccccc5)nc(-c5cccc(-n6c7ccccc7c7ccccc76)c5)n4)c3cc21] | 1283 |
| 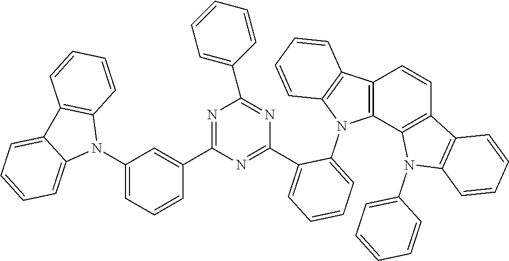 [SMILES: c1ccc(-c2nc(-c3cccc(-n4c5ccccc5c5ccccc54)c3)nc(-c3ccccc3-n3c4ccccc4c4ccc5c6ccccc6n(-c6ccccc6)c5c43)n2)cc1] | 1284 |
| 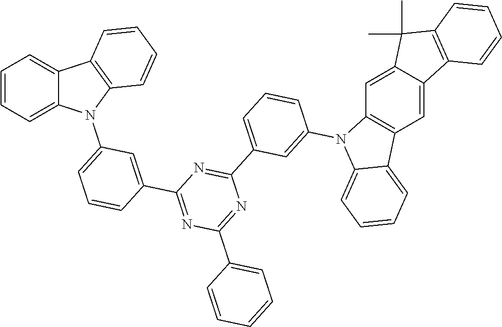 [SMILES: CC1(C)c2ccccc2-c2cc3c4ccccc4n(-c4cccc(-c5nc(-c6ccccc6)nc(-c6cccc(-n7c8ccccc8c8ccccc87)c6)n5)c4)c3cc21] | 1285 |
| 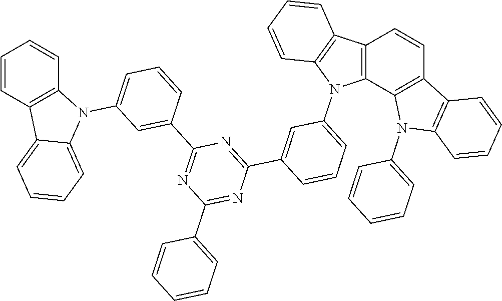 [SMILES: c1ccc(-c2nc(-c3cccc(-n4c5ccccc5c5ccccc54)c3)nc(-c3cccc(-n4c5ccccc5c5ccc6c7ccccc7n(-c7ccccc7)c6c54)c3)n2)cc1] | 1286 |
| 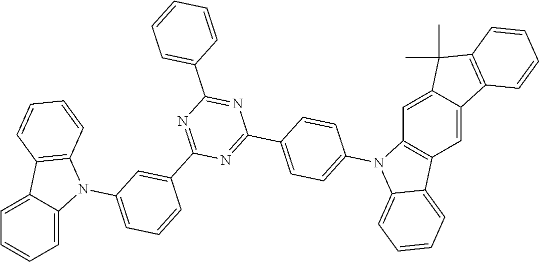 [SMILES: CC1(C)c2ccccc2-c2cc3c4ccccc4n(-c4ccc(-c5nc(-c6ccccc6)nc(-c6cccc(-n7c8ccccc8c8ccccc87)c6)n5)cc4)c3cc21] | 1287 |
| 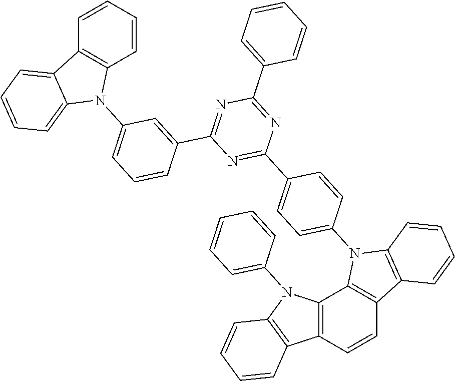 [SMILES: c1ccc(-c2nc(-c3ccc(-n4c5ccccc5c5ccc6c7ccccc7n(-c7ccccc7)c6c54)cc3)nc(-c3cccc(-n4c5ccccc5c5ccccc54)c3)n2)cc1] | 1288 |
| 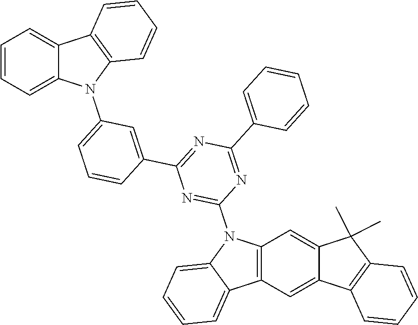 [SMILES: CC1(C)c2ccccc2-c2cc3c4ccccc4n(-c4nc(-c5ccccc5)nc(-c5cccc(-n6c7ccccc7c7ccccc76)c5)n4)c3cc21] | 1289 |
| 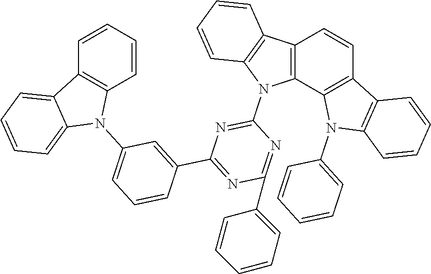 [SMILES: c1ccc(-c2nc(-c3cccc(-n4c5ccccc5c5ccccc54)c3)nc(-n3c4ccccc4c4ccc5c6ccccc6n(-c6ccccc6)c5c43)n2)cc1] | 1290 |
| 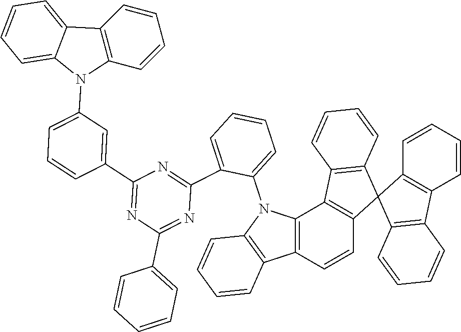 [SMILES: c1ccc(-c2nc(-c3cccc(-n4c5ccccc5c5ccccc54)c3)nc(-c3ccccc3-n3c4ccccc4c4ccc5c(c43)-c3ccccc3C53c4ccccc4-c4ccccc43)n2)cc1] | 1291 |
| 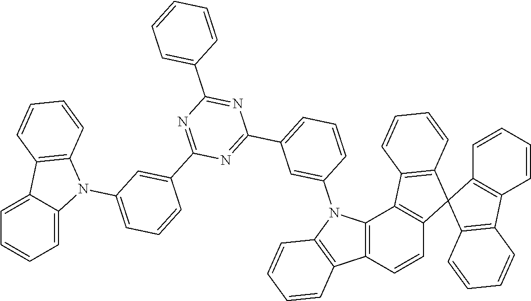 [SMILES: c1ccc(-c2nc(-c3cccc(-n4c5ccccc5c5ccccc54)c3)nc(-c3cccc(-n4c5ccccc5c5ccc6c(c54)-c4ccccc4C64c5ccccc5-c5ccccc54)c3)n2)cc1] | 1292 |
| 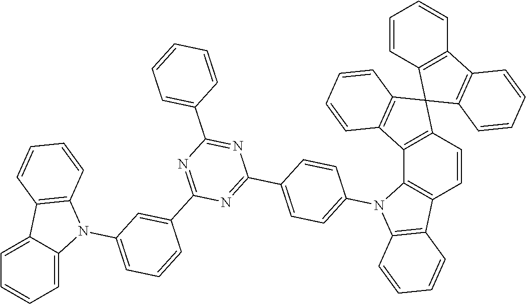 [SMILES: c1ccc(-c2nc(-c3ccc(-n4c5ccccc5c5ccc6c(c54)-c4ccccc4C64c5ccccc5-c5ccccc54)cc3)nc(-c3cccc(-n4c5ccccc5c5ccccc54)c3)n2)cc1] | 1293 |
| 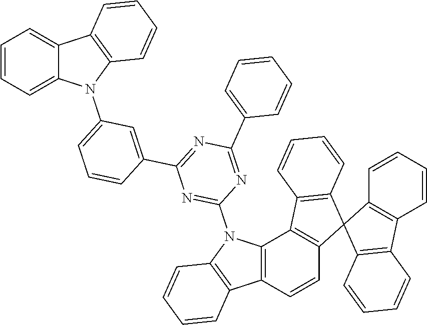 [SMILES: c1ccc(-c2nc(-c3cccc(-n4c5ccccc5c5ccccc54)c3)nc(-n3c4ccccc4c4ccc5c(c43)-c3ccccc3C53c4ccccc4-c4ccccc43)n2)cc1] | 1294 |
| 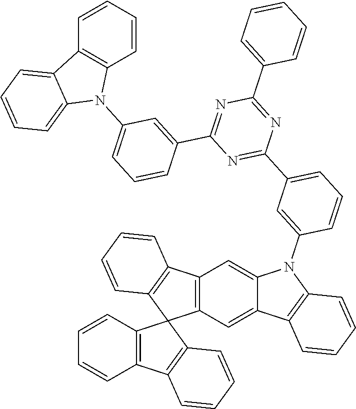 [SMILES: c1ccc(-c2nc(-c3cccc(-n4c5ccccc5c5ccccc54)c3)nc(-c3cccc(-n4c5ccccc5c5cc6c(cc54)-c4ccccc4C64c5ccccc5-c5ccccc54)c3)n2)cc1] | 1295 |
| 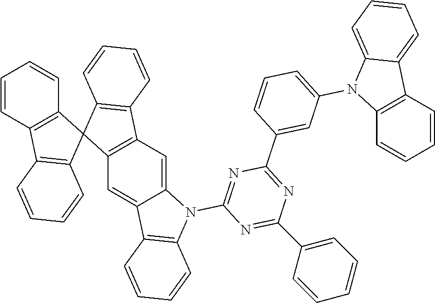 [SMILES: c1ccc(-c2nc(-c3cccc(-n4c5ccccc5c5ccccc54)c3)nc(-n3c4ccccc4c4cc5c(cc43)-c3ccccc3C53c4ccccc4-c4ccccc43)n2)cc1] | 1296 |
| 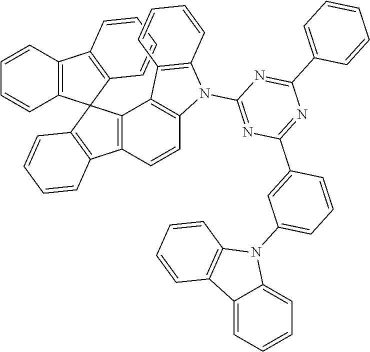 [SMILES: c1ccc(-c2nc(-c3cccc(-n4c5ccccc5c5ccccc54)c3)nc(-n3c4ccccc4c4c5c(ccc43)-c3ccccc3C53c4ccccc4-c4ccccc43)n2)cc1] | 1297 |
| 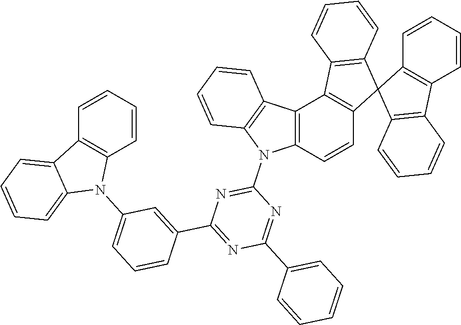 [SMILES: c1ccc(-c2nc(-c3cccc(-n4c5ccccc5c5ccccc54)c3)nc(-n3c4ccccc4c4c5c(ccc43)C3(c4ccccc4-c4ccccc43)c3ccccc3-5)n2)cc1] | 1298 |
| 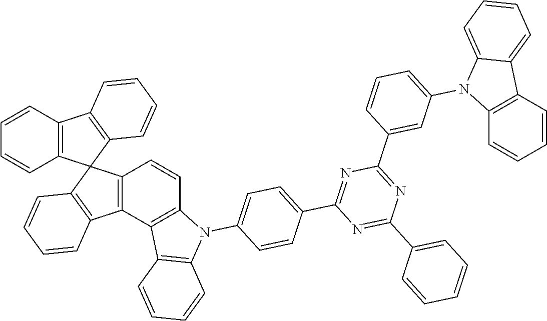 [SMILES: c1ccc(-c2nc(-c3ccc(-n4c5ccccc5c5c6c(ccc54)C4(c5ccccc5-c5ccccc54)c4ccccc4-6)cc3)nc(-c3cccc(-n4c5ccccc5c5ccccc54)c3)n2)cc1] | 1299 |
| 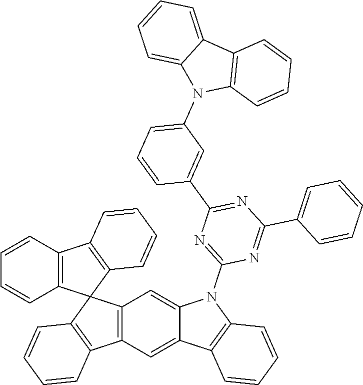 [SMILES: c1ccc(-c2nc(-c3cccc(-n4c5ccccc5c5ccccc54)c3)nc(-n3c4ccccc4c4cc5c(cc43)C3(c4ccccc4-c4ccccc43)c3ccccc3-5)n2)cc1] | 1300 |
| 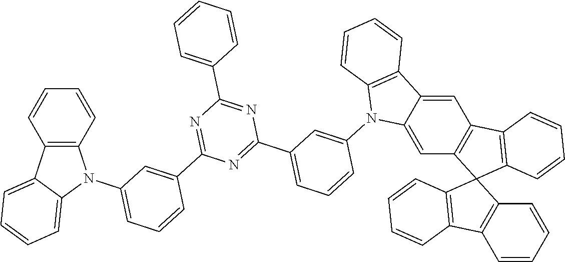 [SMILES: c1ccc(-c2nc(-c3cccc(-n4c5ccccc5c5ccccc54)c3)nc(-c3cccc(-n4c5ccccc5c5cc6c(cc54)C4(c5ccccc5-c5ccccc54)c4ccccc4-6)c3)n2)cc1] | 1301 |
| 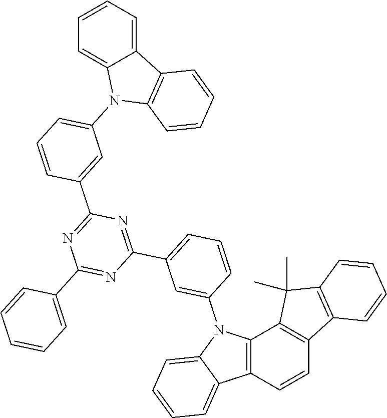 [SMILES: CC1(C)c2ccccc2-c2ccc3c4ccccc4n(-c4cccc(-c5nc(-c6ccccc6)nc(-c6cccc(-n7c8ccccc8c8ccccc87)c6)n5)c4)c3c21] | 1302 |
| 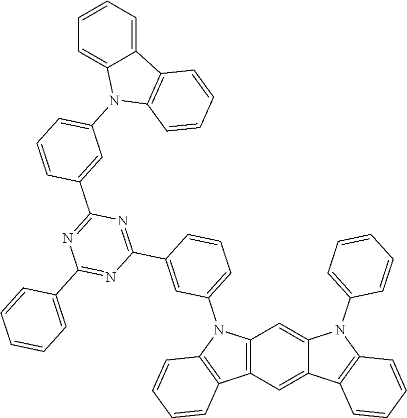 [SMILES: c1ccc(-c2nc(-c3cccc(-n4c5ccccc5c5ccccc54)c3)nc(-c3cccc(-n4c5ccccc5c5cc6c7ccccc7n(-c7ccccc7)c6cc54)c3)n2)cc1] | 1303 |
| 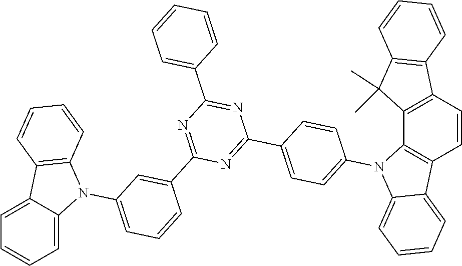 [SMILES: CC1(C)c2ccccc2-c2ccc3c4ccccc4n(-c4ccc(-c5nc(-c6ccccc6)nc(-c6cccc(-n7c8ccccc8c8ccccc87)c6)n5)cc4)c3c21] | 1304 |
| 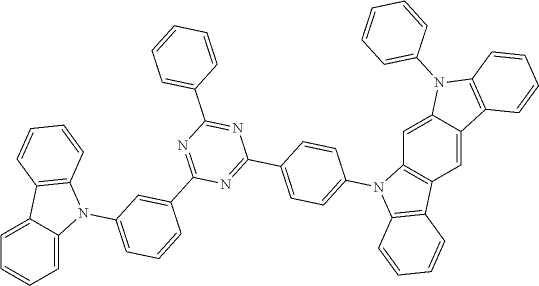 [SMILES: c1ccc(-c2nc(-c3ccc(-n4c5ccccc5c5cc6c7ccccc7n(-c7ccccc7)c6cc54)cc3)nc(-c3cccc(-n4c5ccccc5c5ccccc54)c3)n2)cc1] | 1305 |
| 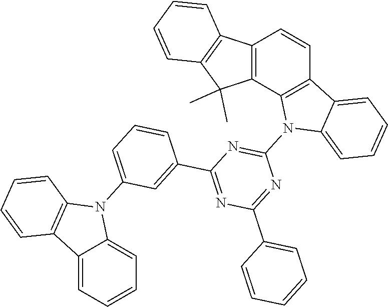 [SMILES: CC1(C)c2ccccc2-c2ccc3c4ccccc4n(-c4nc(-c5ccccc5)nc(-c5cccc(-n6c7ccccc7c7ccccc76)c5)n4)c3c21] | 1306 |
| 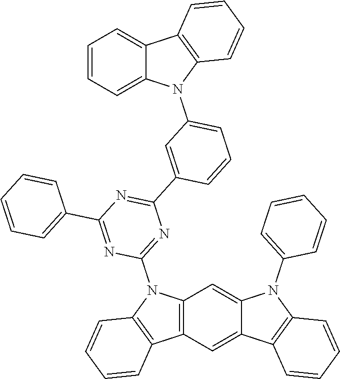 [SMILES: c1ccc(-c2nc(-c3cccc(-n4c5ccccc5c5ccccc54)c3)nc(-n3c4ccccc4c4cc5c6ccccc6n(-c6ccccc6)c5cc43)n2)cc1] | 1307 |
| 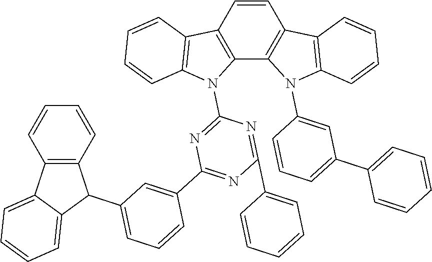 [SMILES: c1ccc(-c2cccc(-n3c4ccccc4c4ccc5c6ccccc6n(-c6nc(-c7ccccc7)nc(-c7cccc(C8c9ccccc9-c9ccccc98)c7)n6)c5c43)c2)cc1] | 1308 |
| 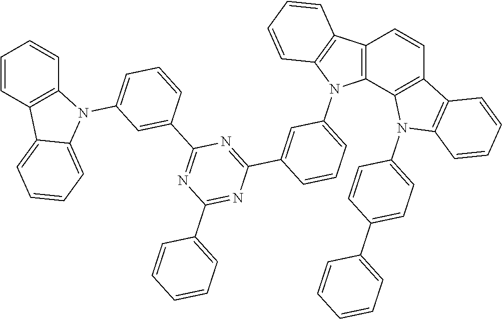 [SMILES: c1ccc(-c2ccc(-n3c4ccccc4c4ccc5c6ccccc6n(-c6cccc(-c7nc(-c8ccccc8)nc(-c8cccc(-n9c%10ccccc%10c%10ccccc%109)c8)n7)c6)c5c43)cc2)cc1] | 1309 |
| 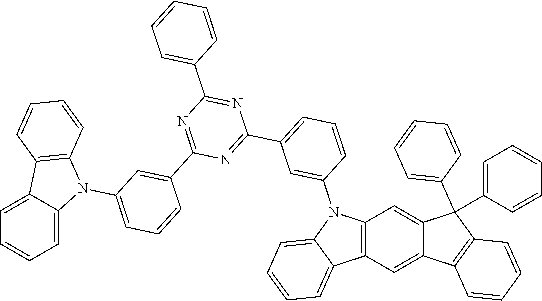 [SMILES: c1ccc(-c2nc(-c3cccc(-n4c5ccccc5c5ccccc54)c3)nc(-c3cccc(-n4c5ccccc5c5cc6c(cc54)C(c4ccccc4)(c4ccccc4)c4ccccc4-6)c3)n2)cc1] | 1310 |
| 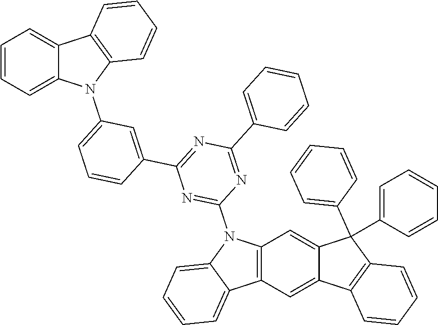 [SMILES: c1ccc(-c2nc(-c3cccc(-n4c5ccccc5c5ccccc54)c3)nc(-n3c4ccccc4c4cc5c(cc43)C(c3ccccc3)(c3ccccc3)c3ccccc3-5)n2)cc1] | 1311 |
| 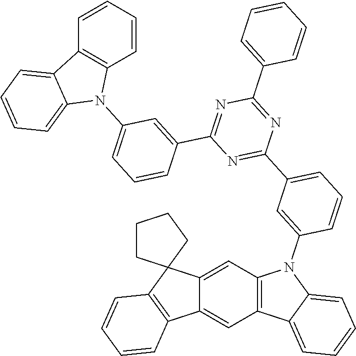 [SMILES: c1ccc(-c2nc(-c3cccc(-n4c5ccccc5c5ccccc54)c3)nc(-c3cccc(-n4c5ccccc5c5cc6c(cc54)C4(CCCC4)c4ccccc4-6)c3)n2)cc1] | 1312 |
| 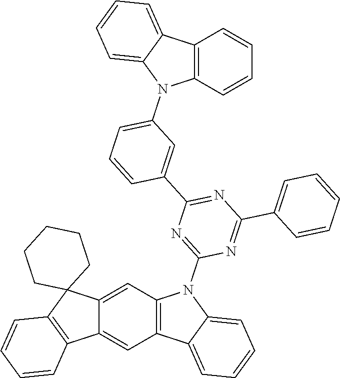 [SMILES: c1ccc(-c2nc(-c3cccc(-n4c5ccccc5c5ccccc54)c3)nc(-n3c4ccccc4c4cc5c(cc43)C3(CCCCC3)c3ccccc3-5)n2)cc1] | 1313 |
| 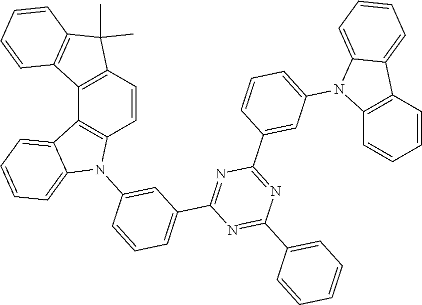 [SMILES: CC1(C)c2ccccc2-c2c1ccc1c2c2ccccc2n1-c1cccc(-c2nc(-c3ccccc3)nc(-c3cccc(-n4c5ccccc5c5ccccc54)c3)n2)c1] | 1314 |
| 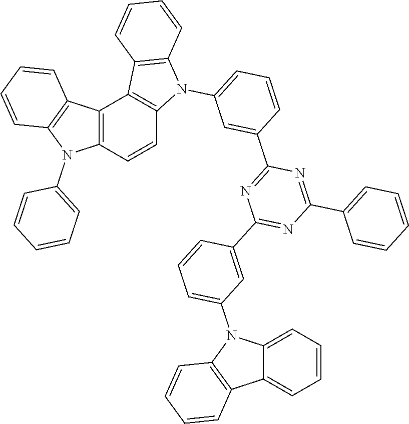 [SMILES: c1ccc(-c2nc(-c3cccc(-n4c5ccccc5c5ccccc54)c3)nc(-c3cccc(-n4c5ccccc5c5c6c7ccccc7n(-c7ccccc7)c6ccc54)c3)n2)cc1] | 1315 |
| 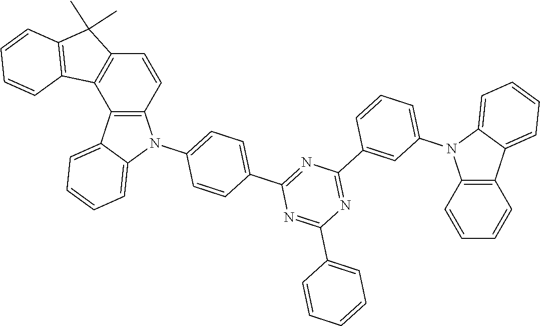 [SMILES: CC1(C)c2ccccc2-c2c1ccc1c2c2ccccc2n1-c1ccc(-c2nc(-c3ccccc3)nc(-c3cccc(-n4c5ccccc5c5ccccc54)c3)n2)cc1] | 1316 |
| 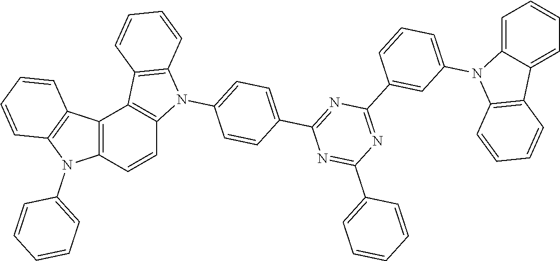 [SMILES: c1ccc(-c2nc(-c3ccc(-n4c5ccccc5c5c6c7ccccc7n(-c7ccccc7)c6ccc54)cc3)nc(-c3cccc(-n4c5ccccc5c5ccccc54)c3)n2)cc1] | 1317 |
| 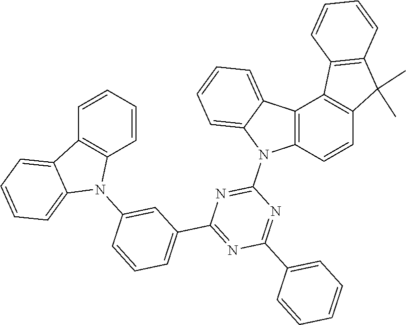 [SMILES: CC1(C)c2ccccc2-c2c1ccc1c2c2ccccc2n1-c1nc(-c2ccccc2)nc(-c2cccc(-n3c4ccccc4c4ccccc43)c2)n1] | 1318 |
| 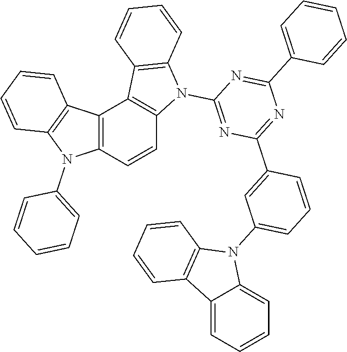 [SMILES: c1ccc(-c2nc(-c3cccc(-n4c5ccccc5c5ccccc54)c3)nc(-n3c4ccccc4c4c5c6ccccc6n(-c6ccccc6)c5ccc43)n2)cc1] | 1319 |
| 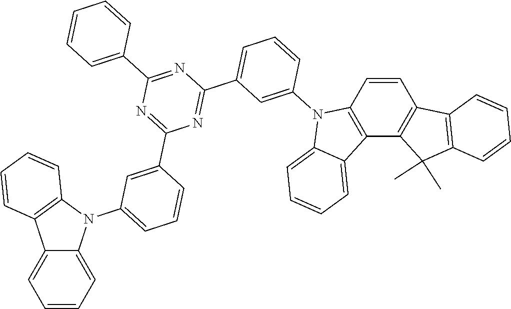 [SMILES: CC1(C)c2ccccc2-c2ccc3c(c21)c1ccccc1n3-c1cccc(-c2nc(-c3ccccc3)nc(-c3cccc(-n4c5ccccc5c5ccccc54)c3)n2)c1] | 1320 |
| 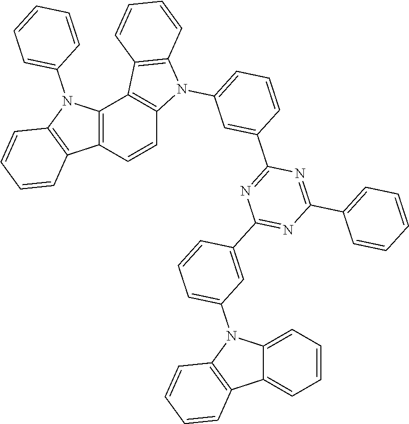 [SMILES: c1ccc(-c2nc(-c3cccc(-n4c5ccccc5c5ccccc54)c3)nc(-c3cccc(-n4c5ccccc5c5c4ccc4c6ccccc6n(-c6ccccc6)c45)c3)n2)cc1] | 1321 |
| 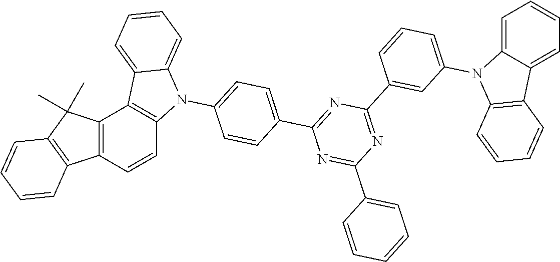 [SMILES: CC1(C)c2ccccc2-c2ccc3c(c21)c1ccccc1n3-c1ccc(-c2nc(-c3ccccc3)nc(-c3cccc(-n4c5ccccc5c5ccccc54)c3)n2)cc1] | 1322 |
| 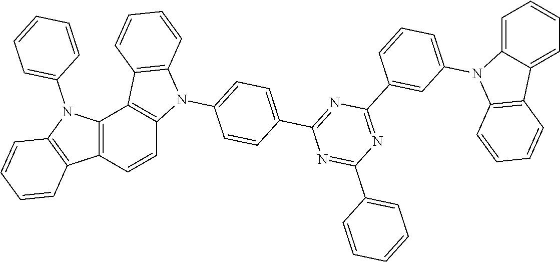 [SMILES: c1ccc(-c2nc(-c3ccc(-n4c5ccccc5c5c4ccc4c6ccccc6n(-c6ccccc6)c45)cc3)nc(-c3cccc(-n4c5ccccc5c5ccccc54)c3)n2)cc1] | 1323 |
| 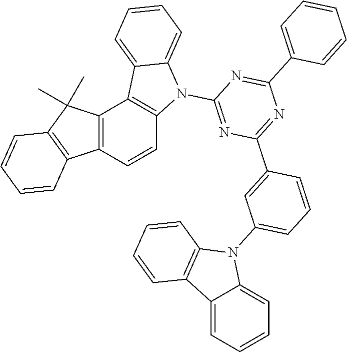 [SMILES: CC1(C)c2ccccc2-c2ccc3c(c21)c1ccccc1n3-c1nc(-c2ccccc2)nc(-c2cccc(-n3c4ccccc4c4ccccc43)c2)n1] | 1324 |
| 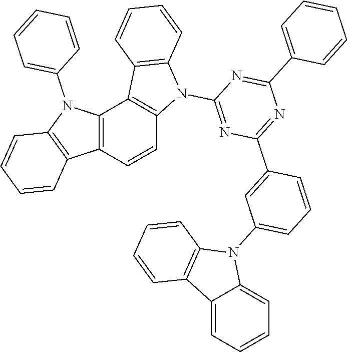 [SMILES: c1ccc(-c2nc(-c3cccc(-n4c5ccccc5c5ccccc54)c3)nc(-n3c4ccccc4c4c3ccc3c5ccccc5n(-c5ccccc5)c34)n2)cc1] | 1325 |
| 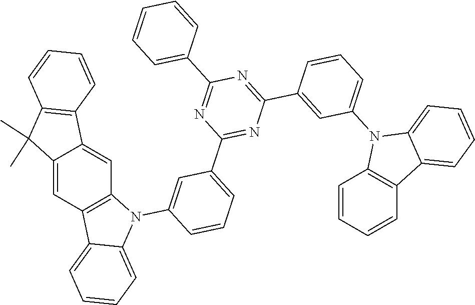 [SMILES: CC1(C)c2ccccc2-c2cc3c(cc21)c1ccccc1n3-c1cccc(-c2nc(-c3ccccc3)nc(-c3cccc(-n4c5ccccc5c5ccccc54)c3)n2)c1] | 1326 |
| 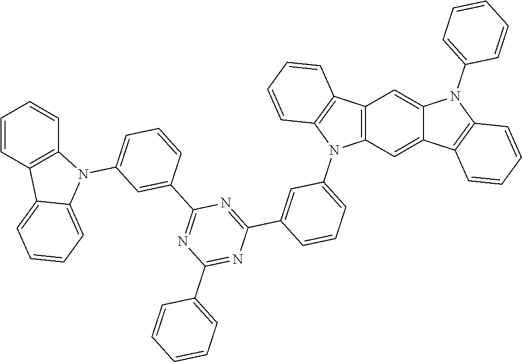 [SMILES: c1ccc(-c2nc(-c3cccc(-n4c5ccccc5c5ccccc54)c3)nc(-c3cccc(-n4c5ccccc5c5cc6c(cc54)c4ccccc4n6-c4ccccc4)c3)n2)cc1] | 1327 |
| 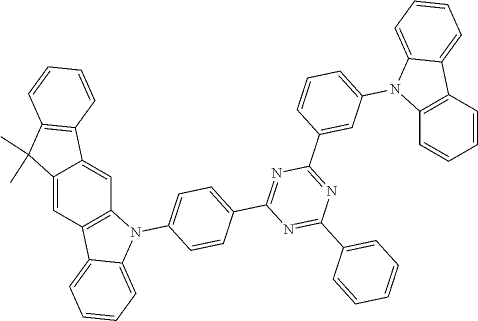 [SMILES: CC1(C)c2ccccc2-c2cc3c(cc21)c1ccccc1n3-c1ccc(-c2nc(-c3ccccc3)nc(-c3cccc(-n4c5ccccc5c5ccccc54)c3)n2)cc1] | 1328 |
| 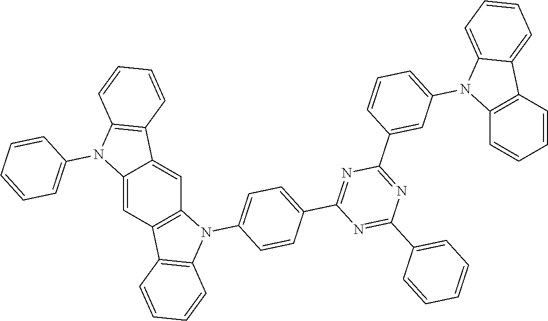 [SMILES: c1ccc(-c2nc(-c3ccc(-n4c5ccccc5c5cc6c(cc54)c4ccccc4n6-c4ccccc4)cc3)nc(-c3cccc(-n4c5ccccc5c5ccccc54)c3)n2)cc1] | 1329 |
| 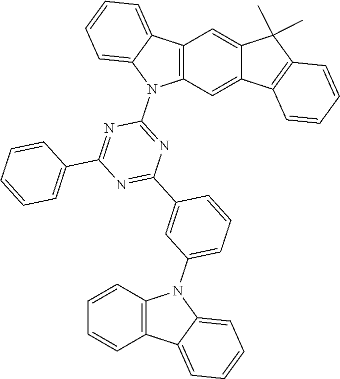 [SMILES: CC1(C)c2ccccc2-c2cc3c(cc21)c1ccccc1n3-c1nc(-c2ccccc2)nc(-c2cccc(-n3c4ccccc4c4ccccc43)c2)n1] | 1330 |
| 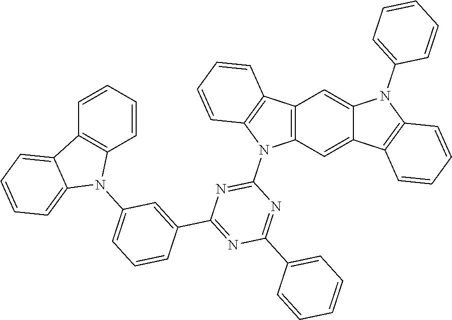 [SMILES: c1ccc(-c2nc(-c3cccc(-n4c5ccccc5c5ccccc54)c3)nc(-n3c4ccccc4c4cc5c(cc43)c3ccccc3n5-c3ccccc3)n2)cc1] | 1331 |
| 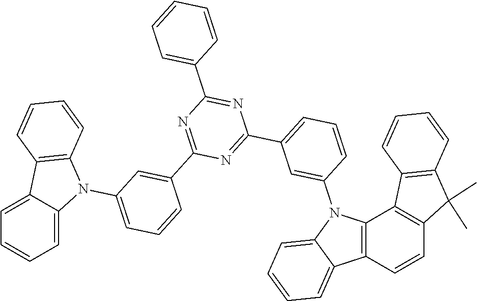 [SMILES: CC1(C)c2ccccc2-c2c1ccc1c3ccccc3n(-c3cccc(-c4nc(-c5ccccc5)nc(-c5cccc(-n6c7ccccc7c7ccccc76)c5)n4)c3)c21] | 1332 |
| 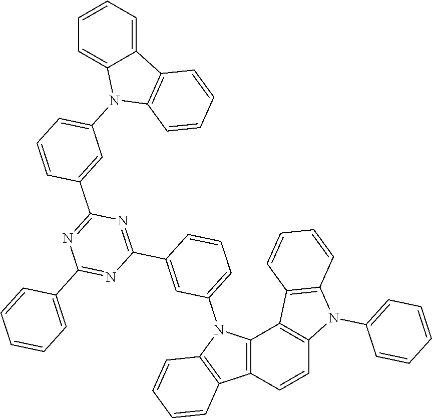 [SMILES: c1ccc(-c2nc(-c3cccc(-n4c5ccccc5c5ccccc54)c3)nc(-c3cccc(-n4c5ccccc5c5ccc6c(c7ccccc7n6-c6ccccc6)c54)c3)n2)cc1] | 1333 |
| 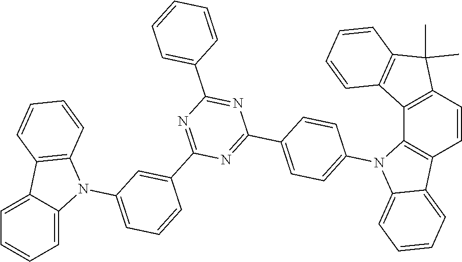 [SMILES: CC1(C)c2ccccc2-c2c1ccc1c3ccccc3n(-c3ccc(-c4nc(-c5ccccc5)nc(-c5cccc(-n6c7ccccc7c7ccccc76)c5)n4)cc3)c21] | 1334 |
| 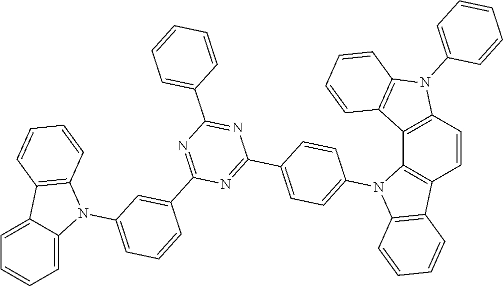 [SMILES: c1ccc(-c2nc(-c3ccc(-n4c5ccccc5c5ccc6c(c7ccccc7n6-c6ccccc6)c54)cc3)nc(-c3cccc(-n4c5ccccc5c5ccccc54)c3)n2)cc1] | 1335 |
| 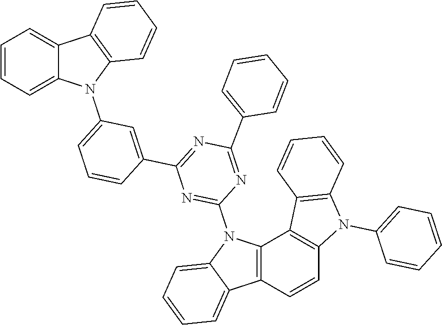 [SMILES: c1ccc(-c2nc(-c3cccc(-n4c5ccccc5c5ccccc54)c3)nc(-n3c4ccccc4c4ccc5c(c6ccccc6n5-c5ccccc5)c43)n2)cc1] | 1336 |
| 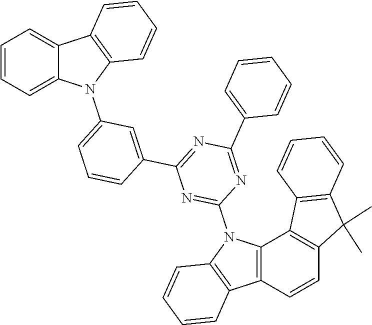 [SMILES: CC1(C)c2ccccc2-c2c1ccc1c3ccccc3n(-c3nc(-c4ccccc4)nc(-c4cccc(-n5c6ccccc6c6ccccc65)c4)n3)c21] | 1337 |
| 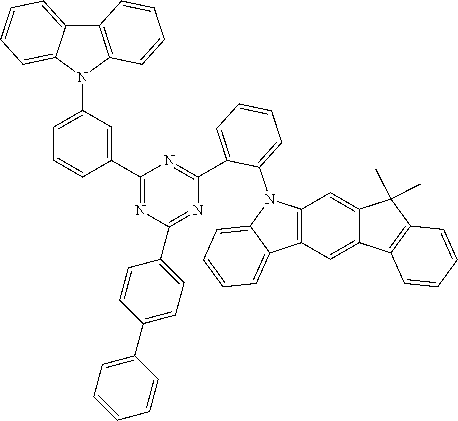 [SMILES: CC1(C)c2ccccc2-c2cc3c4ccccc4n(-c4ccccc4-c4nc(-c5ccc(-c6ccccc6)cc5)nc(-c5cccc(-n6c7ccccc7c7ccccc76)c5)n4)c3cc21] | 1338 |
| 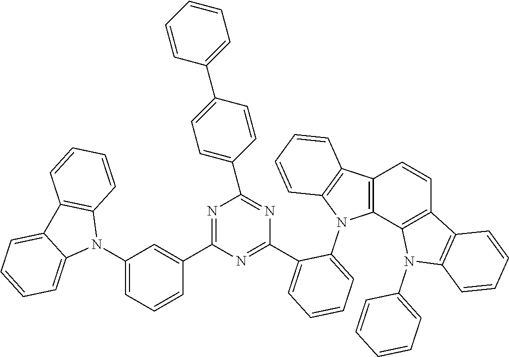 [SMILES: c1ccc(-c2ccc(-c3nc(-c4cccc(-n5c6ccccc6c6ccccc65)c4)nc(-c4ccccc4-n4c5ccccc5c5ccc6c7ccccc7n(-c7ccccc7)c6c54)n3)cc2)cc1] | 1339 |
| 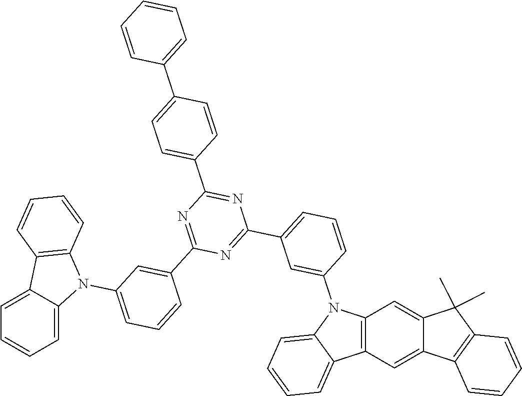 [SMILES: CC1(C)c2ccccc2-c2cc3c4ccccc4n(-c4cccc(-c5nc(-c6ccc(-c7ccccc7)cc6)nc(-c6cccc(-n7c8ccccc8c8ccccc87)c6)n5)c4)c3cc21] | 1340 |
| 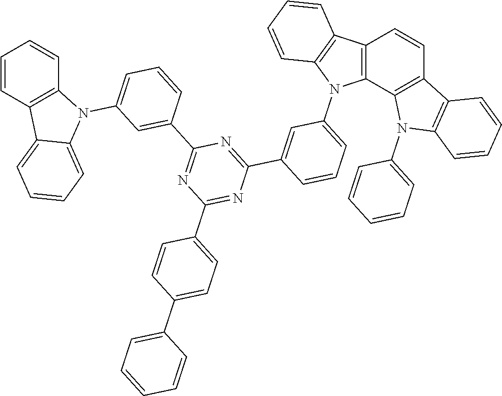 [SMILES: c1ccc(-c2ccc(-c3nc(-c4cccc(-n5c6ccccc6c6ccccc65)c4)nc(-c4cccc(-n5c6ccccc6c6ccc7c8ccccc8n(-c8ccccc8)c7c65)c4)n3)cc2)cc1] | 1341 |
| 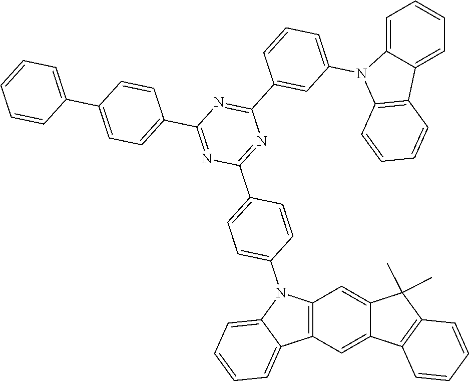 [SMILES: CC1(C)c2ccccc2-c2cc3c4ccccc4n(-c4ccc(-c5nc(-c6ccc(-c7ccccc7)cc6)nc(-c6cccc(-n7c8ccccc8c8ccccc87)c6)n5)cc4)c3cc21] | 1342 |
| 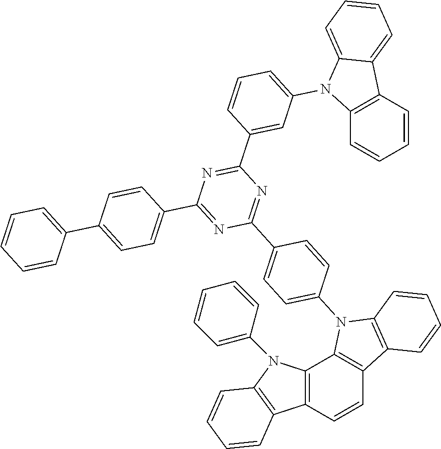 [SMILES: c1ccc(-c2ccc(-c3nc(-c4ccc(-n5c6ccccc6c6ccc7c8ccccc8n(-c8ccccc8)c7c65)cc4)nc(-c4cccc(-n5c6ccccc6c6ccccc65)c4)n3)cc2)cc1] | 1343 |
| 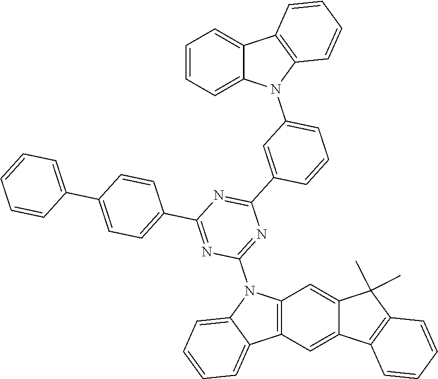 [SMILES: CC1(C)c2ccccc2-c2cc3c4ccccc4n(-c4nc(-c5ccc(-c6ccccc6)cc5)nc(-c5cccc(-n6c7ccccc7c7ccccc76)c5)n4)c3cc21] | 1344 |
| 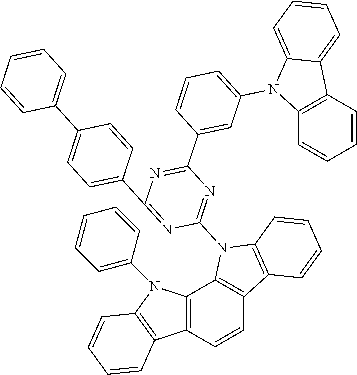 [SMILES: c1ccc(-c2ccc(-c3nc(-c4cccc(-n5c6ccccc6c6ccccc65)c4)nc(-n4c5ccccc5c5ccc6c7ccccc7n(-c7ccccc7)c6c54)n3)cc2)cc1] | 1345 |
| 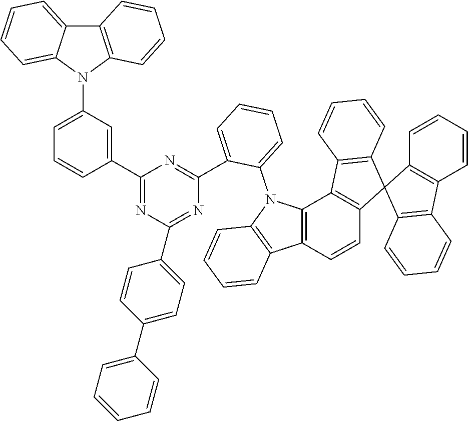 [SMILES: c1ccc(-c2ccc(-c3nc(-c4cccc(-n5c6ccccc6c6ccccc65)c4)nc(-c4ccccc4-n4c5ccccc5c5ccc6c(c54)-c4ccccc4C64c5ccccc5-c5ccccc54)n3)cc2)cc1] | 1346 |
| 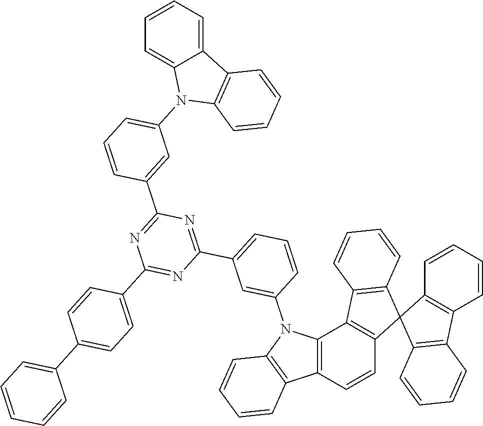 [SMILES: c1ccc(-c2ccc(-c3nc(-c4cccc(-n5c6ccccc6c6ccccc65)c4)nc(-c4cccc(-n5c6ccccc6c6ccc7c(c65)-c5ccccc5C75c6ccccc6-c6ccccc65)c4)n3)cc2)cc1] | 1346 |
| 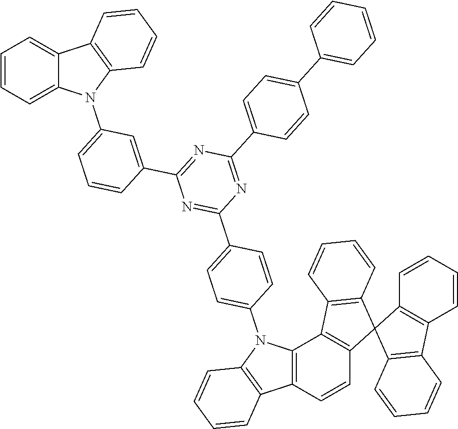 [SMILES: c1ccc(-c2ccc(-c3nc(-c4ccc(-n5c6ccccc6c6ccc7c(c65)-c5ccccc5C75c6ccccc6-c6ccccc65)cc4)nc(-c4cccc(-n5c6ccccc6c6ccccc65)c4)n3)cc2)cc1] | 1347 |
| 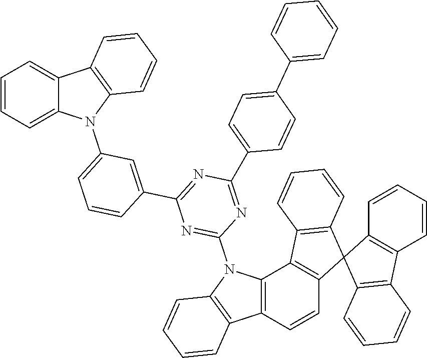 [SMILES: c1ccc(-c2ccc(-c3nc(-c4cccc(-n5c6ccccc6c6ccccc65)c4)nc(-n4c5ccccc5c5ccc6c(c54)-c4ccccc4C64c5ccccc5-c5ccccc54)n3)cc2)cc1] | 1348 |
| 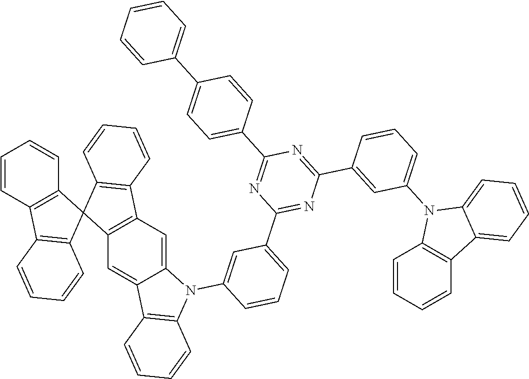 [SMILES: c1ccc(-c2ccc(-c3nc(-c4cccc(-n5c6ccccc6c6ccccc65)c4)nc(-c4cccc(-n5c6ccccc6c6cc7c(cc65)-c5ccccc5C75c6ccccc6-c6ccccc65)c4)n3)cc2)cc1] | 1349 |
| 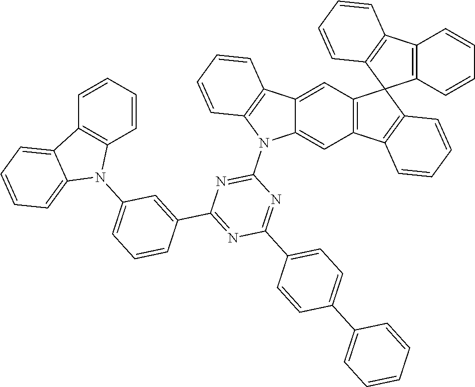 [SMILES: c1ccc(-c2ccc(-c3nc(-c4cccc(-n5c6ccccc6c6ccccc65)c4)nc(-n4c5ccccc5c5cc6c(cc54)-c4ccccc4C64c5ccccc5-c5ccccc54)n3)cc2)cc1] | 1350 |
| 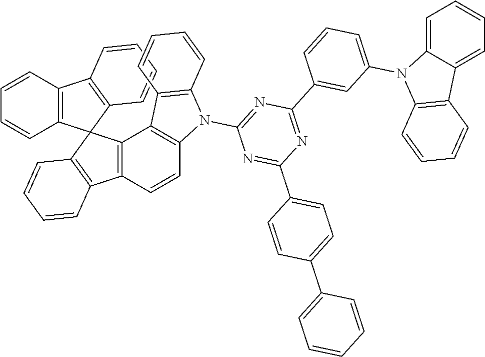 [SMILES: c1ccc(-c2ccc(-c3nc(-c4cccc(-n5c6ccccc6c6ccccc65)c4)nc(-n4c5ccccc5c5c6c(ccc54)-c4ccccc4C64c5ccccc5-c5ccccc54)n3)cc2)cc1] | 1351 |
| 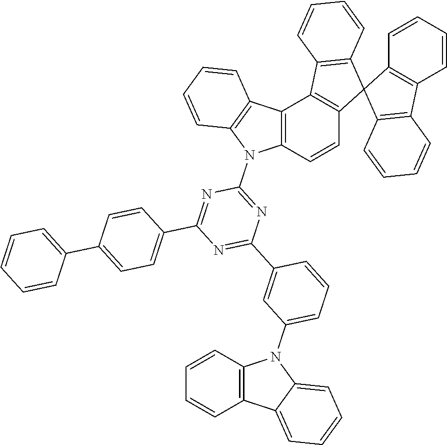 [SMILES: c1ccc(-c2ccc(-c3nc(-c4cccc(-n5c6ccccc6c6ccccc65)c4)nc(-n4c5ccccc5c5c6c(ccc54)C4(c5ccccc5-c5ccccc54)c4ccccc4-6)n3)cc2)cc1] | 1352 |
| 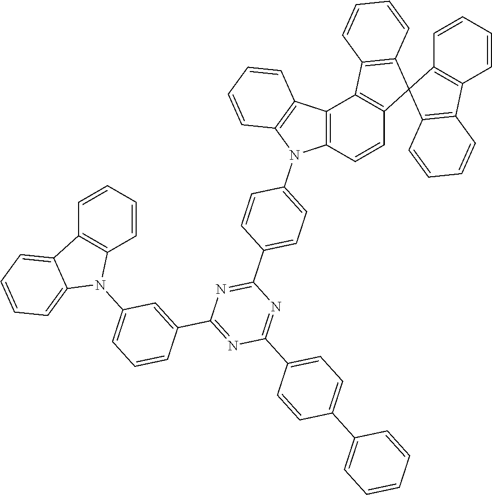 [SMILES: c1ccc(-c2ccc(-c3nc(-c4ccc(-n5c6ccccc6c6c7c(ccc65)C5(c6ccccc6-c6ccccc65)c5ccccc5-7)cc4)nc(-c4cccc(-n5c6ccccc6c6ccccc65)c4)n3)cc2)cc1] | 1353 |
| 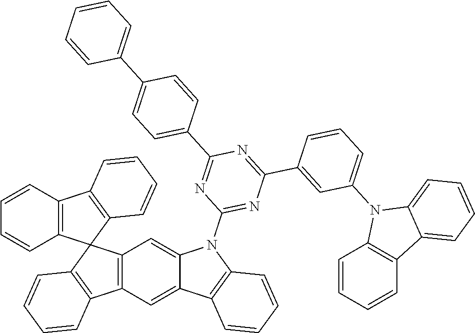 [SMILES: c1ccc(-c2ccc(-c3nc(-c4cccc(-n5c6ccccc6c6ccccc65)c4)nc(-n4c5ccccc5c5cc6c(cc54)C4(c5ccccc5-c5ccccc54)c4ccccc4-6)n3)cc2)cc1] | 1354 |
| 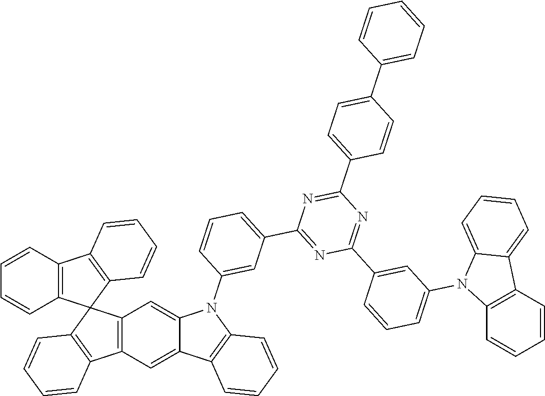 [SMILES: c1ccc(-c2ccc(-c3nc(-c4cccc(-n5c6ccccc6c6ccccc65)c4)nc(-c4cccc(-n5c6ccccc6c6cc7c(cc65)C5(c6ccccc6-c6ccccc65)c5ccccc5-7)c4)n3)cc2)cc1] | 1355 |
| 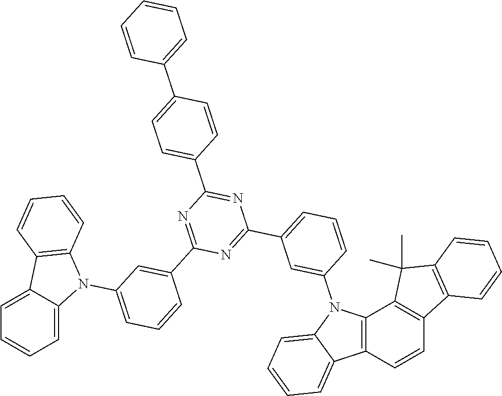 [SMILES: CC1(C)c2ccccc2-c2ccc3c4ccccc4n(-c4cccc(-c5nc(-c6ccc(-c7ccccc7)cc6)nc(-c6cccc(-n7c8ccccc8c8ccccc87)c6)n5)c4)c3c21] | 1356 |
| 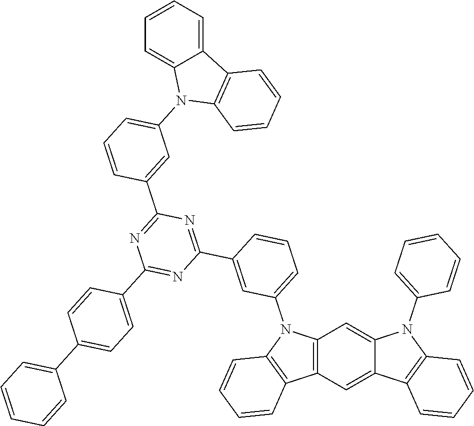 [SMILES: c1ccc(-c2ccc(-c3nc(-c4cccc(-n5c6ccccc6c6ccccc65)c4)nc(-c4cccc(-n5c6ccccc6c6cc7c8ccccc8n(-c8ccccc8)c7cc65)c4)n3)cc2)cc1] | 1357 |
| 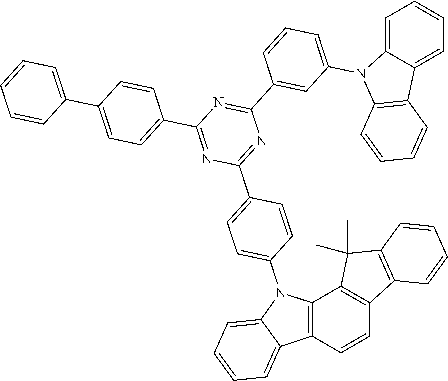 [SMILES: CC1(C)c2ccccc2-c2ccc3c4ccccc4n(-c4ccc(-c5nc(-c6ccc(-c7ccccc7)cc6)nc(-c6cccc(-n7c8ccccc8c8ccccc87)c6)n5)cc4)c3c21] | 1358 |
| 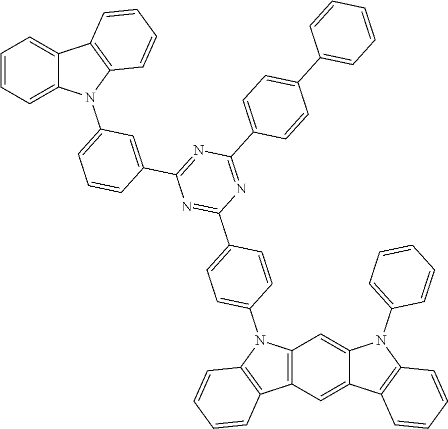 [SMILES: c1ccc(-c2ccc(-c3nc(-c4ccc(-n5c6ccccc6c6cc7c8ccccc8n(-c8ccccc8)c7cc65)cc4)nc(-c4cccc(-n5c6ccccc6c6ccccc65)c4)n3)cc2)cc1] | 1359 |
| 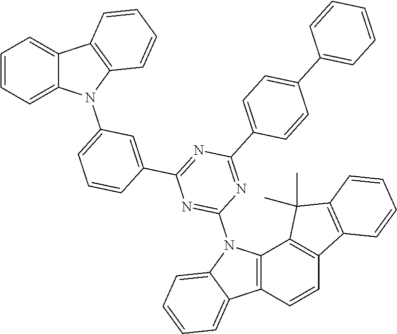 [SMILES: CC1(C)c2ccccc2-c2ccc3c4ccccc4n(-c4nc(-c5ccc(-c6ccccc6)cc5)nc(-c5cccc(-n6c7ccccc7c7ccccc76)c5)n4)c3c21] | 1360 |
| 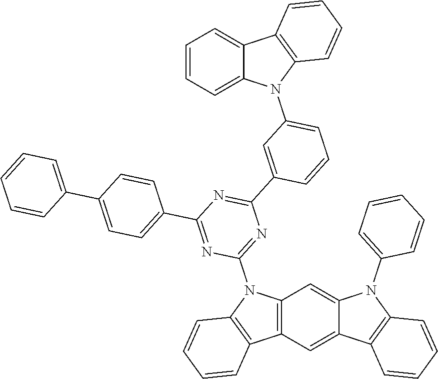 [SMILES: c1ccc(-c2ccc(-c3nc(-c4cccc(-n5c6ccccc6c6ccccc65)c4)nc(-n4c5ccccc5c5cc6c7ccccc7n(-c7ccccc7)c6cc54)n3)cc2)cc1] | 1361 |
| 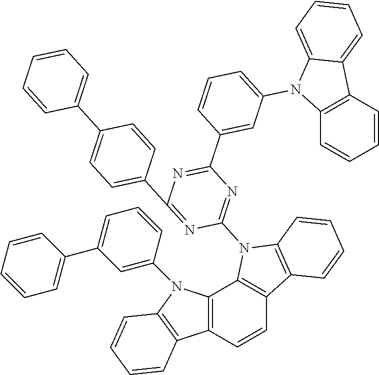 [SMILES: c1ccc(-c2ccc(-c3nc(-c4cccc(-n5c6ccccc6c6ccccc65)c4)nc(-n4c5ccccc5c5ccc6c7ccccc7n(-c7cccc(-c8ccccc8)c7)c6c54)n3)cc2)cc1] | 1362 |
| 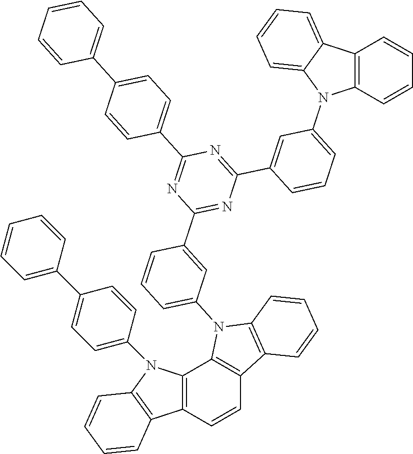 [SMILES: c1ccc(-c2ccc(-c3nc(-c4cccc(-n5c6ccccc6c6ccccc65)c4)nc(-c4cccc(-n5c6ccccc6c6ccc7c8ccccc8n(-c8ccc(-c9ccccc9)cc8)c7c65)c4)n3)cc2)cc1] | 1363 |
| 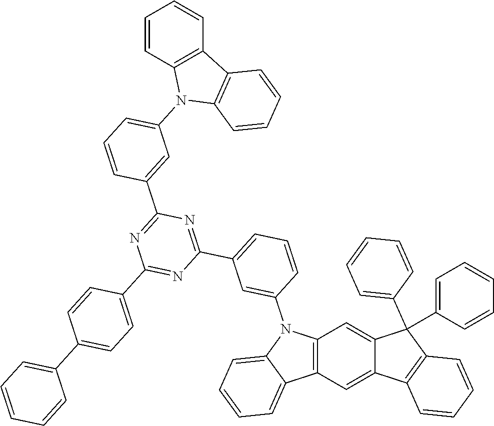 [SMILES: c1ccc(-c2ccc(-c3nc(-c4cccc(-n5c6ccccc6c6ccccc65)c4)nc(-c4cccc(-n5c6ccccc6c6cc7c(cc65)C(c5ccccc5)(c5ccccc5)c5ccccc5-7)c4)n3)cc2)cc1] | 1364 |
| 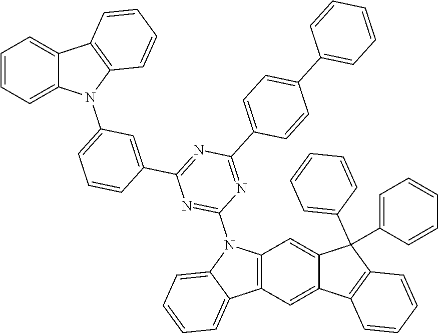 [SMILES: c1ccc(-c2ccc(-c3nc(-c4cccc(-n5c6ccccc6c6ccccc65)c4)nc(-n4c5ccccc5c5cc6c(cc54)C(c4ccccc4)(c4ccccc4)c4ccccc4-6)n3)cc2)cc1] | 1365 |
| 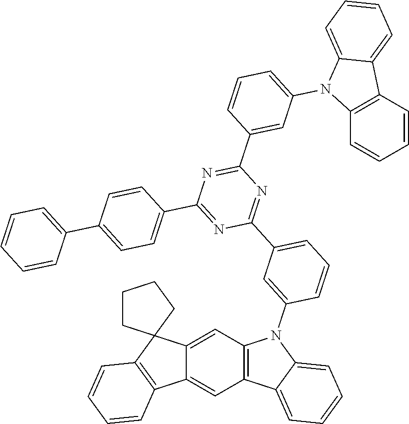 [SMILES: c1ccc(-c2ccc(-c3nc(-c4cccc(-n5c6ccccc6c6ccccc65)c4)nc(-c4cccc(-n5c6ccccc6c6cc7c(cc65)C5(CCCC5)c5ccccc5-7)c4)n3)cc2)cc1] | 1366 |
| 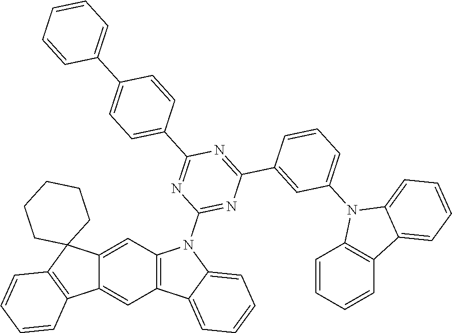 [SMILES: c1ccc(-c2ccc(-c3nc(-c4cccc(-n5c6ccccc6c6ccccc65)c4)nc(-n4c5ccccc5c5cc6c(cc54)C4(CCCCC4)c4ccccc4-6)n3)cc2)cc1] | 1367 |
| 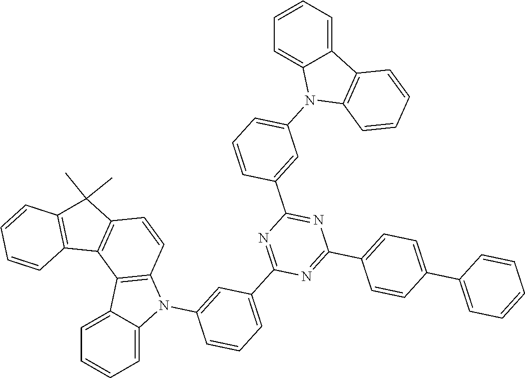 [SMILES: CC1(C)c2ccccc2-c2c1ccc1c2c2ccccc2n1-c1cccc(-c2nc(-c3ccc(-c4ccccc4)cc3)nc(-c3cccc(-n4c5ccccc5c5ccccc54)c3)n2)c1] | 1367 |
| 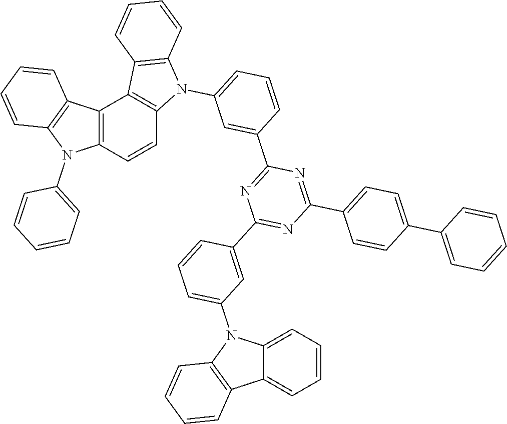 [SMILES: c1ccc(-c2ccc(-c3nc(-c4cccc(-n5c6ccccc6c6ccccc65)c4)nc(-c4cccc(-n5c6ccccc6c6c7c8ccccc8n(-c8ccccc8)c7ccc65)c4)n3)cc2)cc1] | 1368 |
| 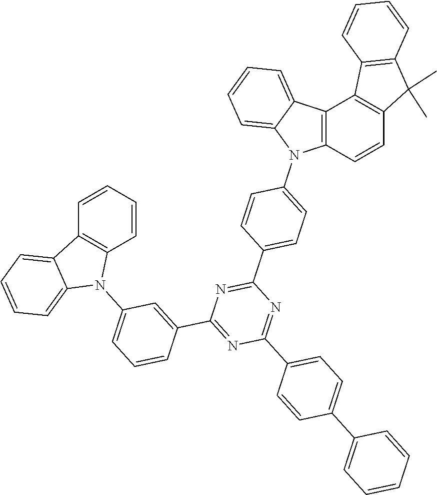 [SMILES: CC1(C)c2ccccc2-c2c1ccc1c2c2ccccc2n1-c1ccc(-c2nc(-c3ccc(-c4ccccc4)cc3)nc(-c3cccc(-n4c5ccccc5c5ccccc54)c3)n2)cc1] | 1369 |
| 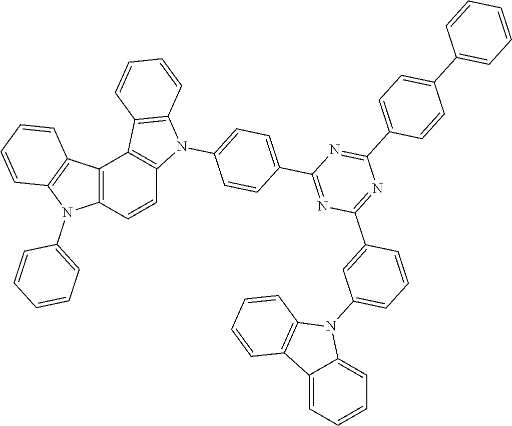 [SMILES: c1ccc(-c2ccc(-c3nc(-c4ccc(-n5c6ccccc6c6c7c8ccccc8n(-c8ccccc8)c7ccc65)cc4)nc(-c4cccc(-n5c6ccccc6c6ccccc65)c4)n3)cc2)cc1] | 1370 |
| 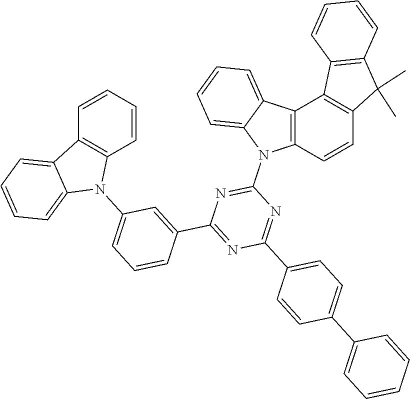 [SMILES: CC1(C)c2ccccc2-c2c1ccc1c2c2ccccc2n1-c1nc(-c2ccc(-c3ccccc3)cc2)nc(-c2cccc(-n3c4ccccc4c4ccccc43)c2)n1] | 1371 |
| 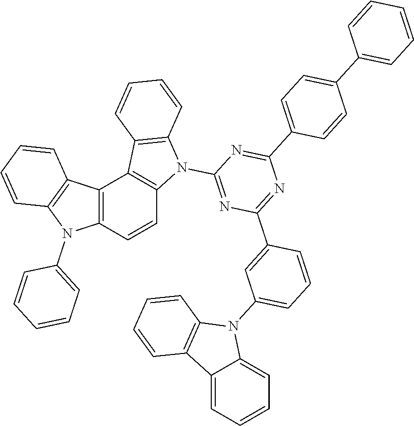 [SMILES: c1ccc(-c2ccc(-c3nc(-c4cccc(-n5c6ccccc6c6ccccc65)c4)nc(-n4c5ccccc5c5c6c7ccccc7n(-c7ccccc7)c6ccc54)n3)cc2)cc1] | 1372 |
| 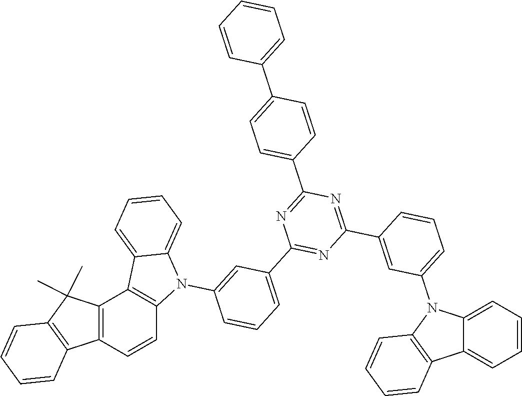 [SMILES: CC1(C)c2ccccc2-c2ccc3c(c21)c1ccccc1n3-c1cccc(-c2nc(-c3ccc(-c4ccccc4)cc3)nc(-c3cccc(-n4c5ccccc5c5ccccc54)c3)n2)c1] | 1373 |
| 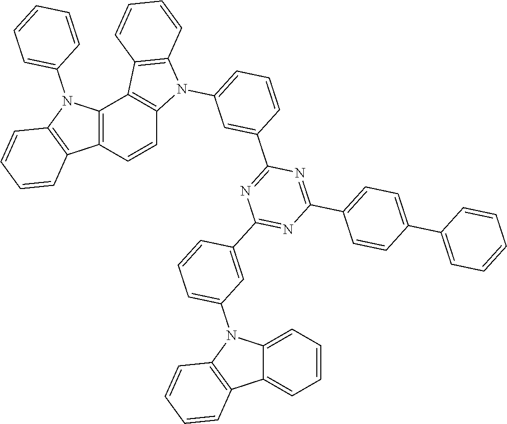 [SMILES: c1ccc(-c2ccc(-c3nc(-c4cccc(-n5c6ccccc6c6ccccc65)c4)nc(-c4cccc(-n5c6ccccc6c6c5ccc5c7ccccc7n(-c7ccccc7)c56)c4)n3)cc2)cc1] | 1374 |
| 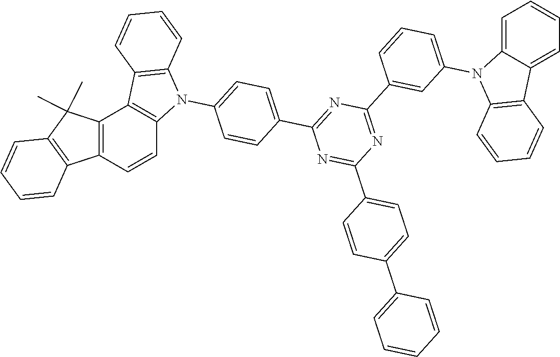 [SMILES: CC1(C)c2ccccc2-c2ccc3c(c21)c1ccccc1n3-c1ccc(-c2nc(-c3ccc(-c4ccccc4)cc3)nc(-c3cccc(-n4c5ccccc5c5ccccc54)c3)n2)cc1] | 1375 |
| 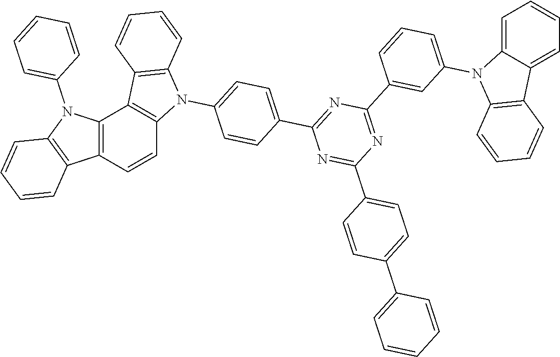 [SMILES: c1ccc(-c2ccc(-c3nc(-c4ccc(-n5c6ccccc6c6c5ccc5c7ccccc7n(-c7ccccc7)c56)cc4)nc(-c4cccc(-n5c6ccccc6c6ccccc65)c4)n3)cc2)cc1] | 1376 |
| 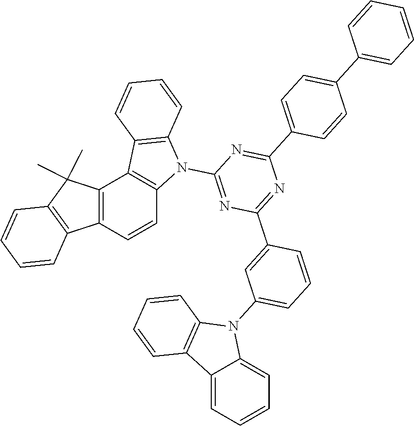 [SMILES: CC1(C)c2ccccc2-c2ccc3c(c21)c1ccccc1n3-c1nc(-c2ccc(-c3ccccc3)cc2)nc(-c2cccc(-n3c4ccccc4c4ccccc43)c2)n1] | 1377 |
| 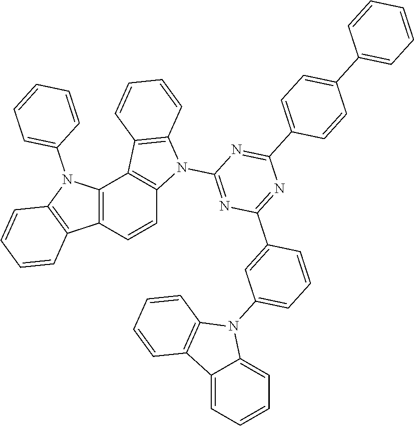 [SMILES: c1ccc(-c2ccc(-c3nc(-c4cccc(-n5c6ccccc6c6ccccc65)c4)nc(-n4c5ccccc5c5c4ccc4c6ccccc6n(-c6ccccc6)c45)n3)cc2)cc1] | 1378 |
| 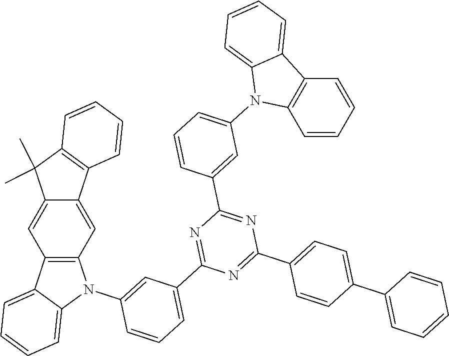 [SMILES: CC1(C)c2ccccc2-c2cc3c(cc21)c1ccccc1n3-c1cccc(-c2nc(-c3ccc(-c4ccccc4)cc3)nc(-c3cccc(-n4c5ccccc5c5ccccc54)c3)n2)c1] | 1379 |
| 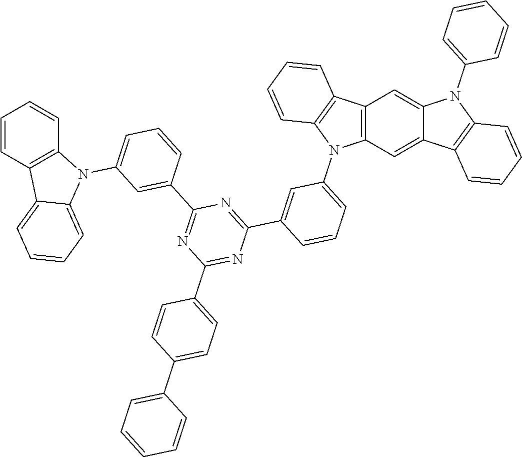 [SMILES: c1ccc(-c2ccc(-c3nc(-c4cccc(-n5c6ccccc6c6ccccc65)c4)nc(-c4cccc(-n5c6ccccc6c6cc7c(cc65)c5ccccc5n7-c5ccccc5)c4)n3)cc2)cc1] | 1380 |
| 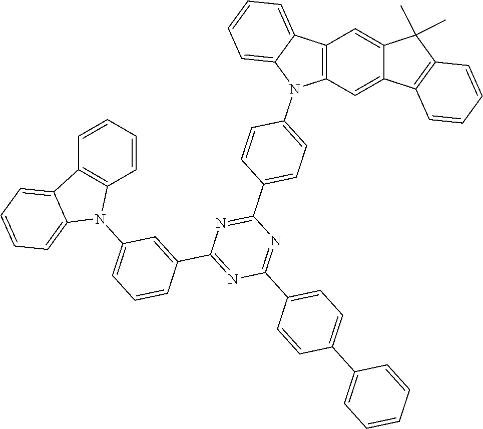 [SMILES: CC1(C)c2ccccc2-c2cc3c(cc21)c1ccccc1n3-c1ccc(-c2nc(-c3ccc(-c4ccccc4)cc3)nc(-c3cccc(-n4c5ccccc5c5ccccc54)c3)n2)cc1] | 1381 |
| 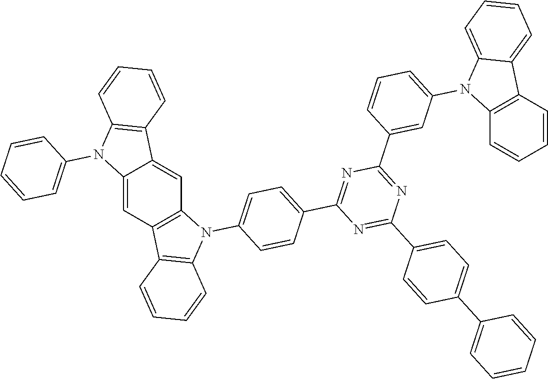 [SMILES: c1ccc(-c2ccc(-c3nc(-c4ccc(-n5c6ccccc6c6cc7c(cc65)c5ccccc5n7-c5ccccc5)cc4)nc(-c4cccc(-n5c6ccccc6c6ccccc65)c4)n3)cc2)cc1] | 1382 |
| 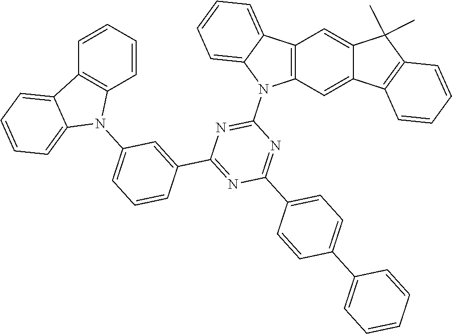 [SMILES: CC1(C)c2ccccc2-c2cc3c(cc21)c1ccccc1n3-c1nc(-c2ccc(-c3ccccc3)cc2)nc(-c2cccc(-n3c4ccccc4c4ccccc43)c2)n1] | 1383 |
| 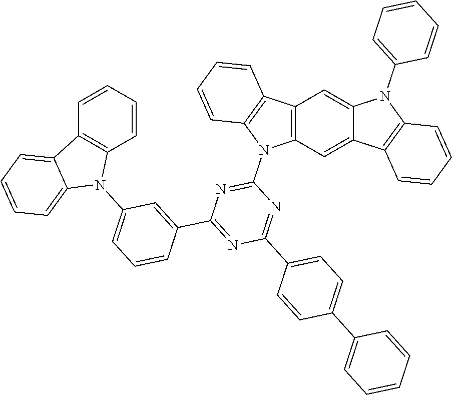 [SMILES: c1ccc(-c2ccc(-c3nc(-c4cccc(-n5c6ccccc6c6ccccc65)c4)nc(-n4c5ccccc5c5cc6c(cc54)c4ccccc4n6-c4ccccc4)n3)cc2)cc1] | 1384 |
| 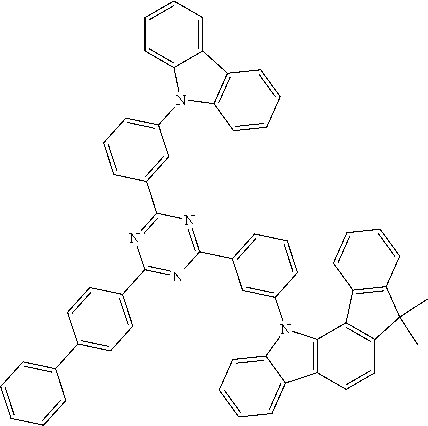 [SMILES: CC1(C)c2ccccc2-c2c1ccc1c3ccccc3n(-c3cccc(-c4nc(-c5ccc(-c6ccccc6)cc5)nc(-c5cccc(-n6c7ccccc7c7ccccc76)c5)n4)c3)c21] | 1385 |
| 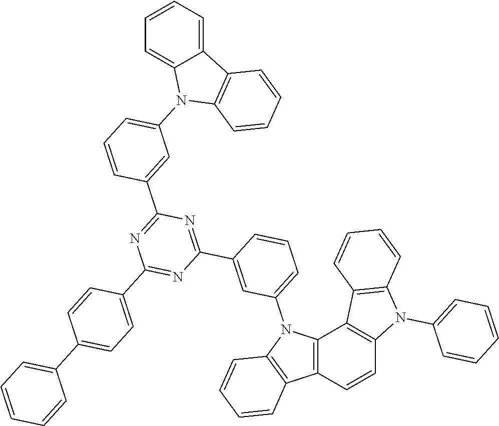 [SMILES: c1ccc(-c2ccc(-c3nc(-c4cccc(-n5c6ccccc6c6ccccc65)c4)nc(-c4cccc(-n5c6ccccc6c6ccc7c(c8ccccc8n7-c7ccccc7)c65)c4)n3)cc2)cc1] | 1386 |
| 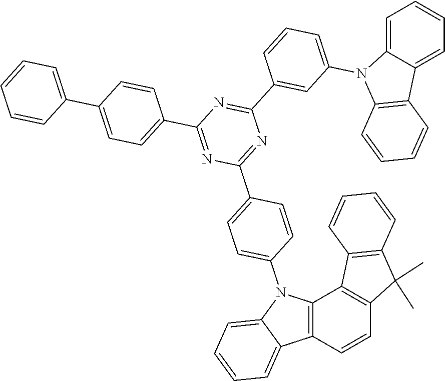 [SMILES: CC1(C)c2ccccc2-c2c1ccc1c3ccccc3n(-c3ccc(-c4nc(-c5ccc(-c6ccccc6)cc5)nc(-c5cccc(-n6c7ccccc7c7ccccc76)c5)n4)cc3)c21] | 1387 |
| 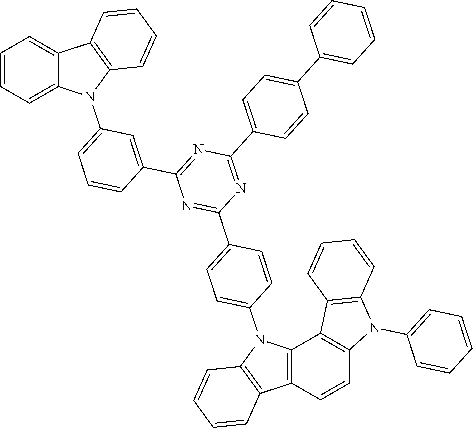 [SMILES: c1ccc(-c2ccc(-c3nc(-c4ccc(-n5c6ccccc6c6ccc7c(c8ccccc8n7-c7ccccc7)c65)cc4)nc(-c4cccc(-n5c6ccccc6c6ccccc65)c4)n3)cc2)cc1] | 1388 |
| 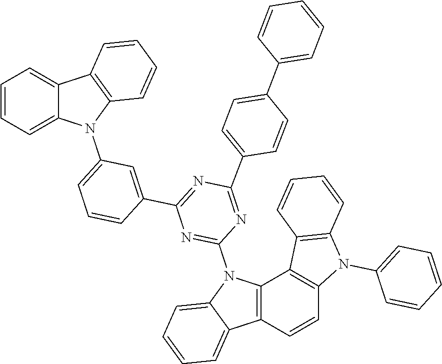 [SMILES: c1ccc(-c2ccc(-c3nc(-c4cccc(-n5c6ccccc6c6ccccc65)c4)nc(-n4c5ccccc5c5ccc6c(c7ccccc7n6-c6ccccc6)c54)n3)cc2)cc1] | 1389 |
| 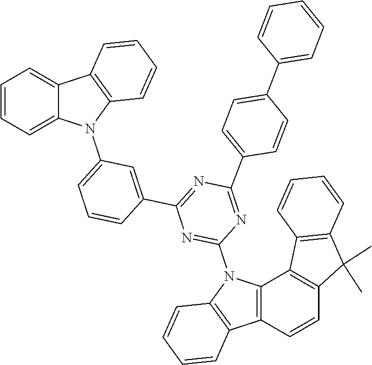 [SMILES: CC1(C)c2ccccc2-c2c1ccc1c3ccccc3n(-c3nc(-c4ccc(-c5ccccc5)cc4)nc(-c4cccc(-n5c6ccccc6c6ccccc65)c4)n3)c21] | 1390 |
| 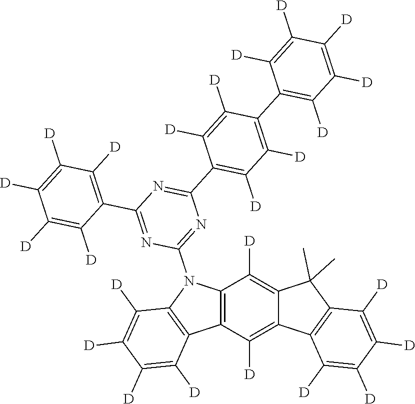 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c5c(c([2H])c43)C(C)(C)c3c([2H])c([2H])c([2H])c([2H])c3-5)n2)c([2H])c1[2H]] | 1391 |
| 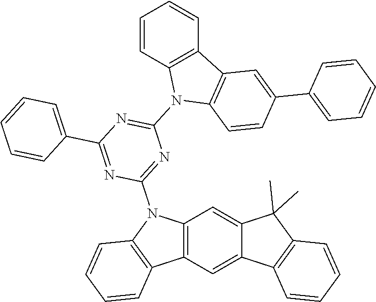 [SMILES: CC1(C)c2ccccc2-c2cc3c4ccccc4n(-c4nc(-c5ccccc5)nc(-n5c6ccccc6c6cc(-c7ccccc7)ccc65)n4)c3cc21] | 1392 |
| 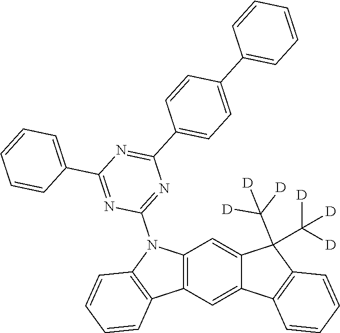 [SMILES: [2H]C([2H])([2H])C1(C([2H])([2H])[2H])c2ccccc2-c2cc3c4ccccc4n(-c4nc(-c5ccccc5)nc(-c5ccc(-c6ccccc6)cc5)n4)c3cc21] | 1393 |
| 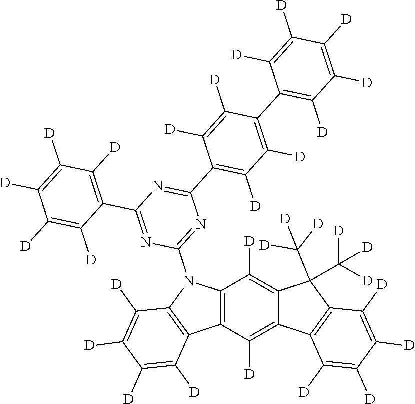 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c5c(c([2H])c43)C(C([2H])([2H])[2H])(C([2H])([2H])[2H])c3c([2H])c([2H])c([2H])c([2H])c3-5)n2)c([2H])c1[2H]] | 1394 |

Particularly suitable compounds of the formulae (1), (1a), (1b), (1c), (1d), (1e), (1f), (1g) and (1h) that are used with preference in combination with at least one compound of the formula (2) in the electroluminescent device of the invention are the compounds E1 to E54 and E60 to E69.

TABLE 2
Compound 3
E1
Compound 63
E2
Compound 111
E3
Compound 113
E4
Compound 117
E5
Compound 118
E6
Compound 121
E7
Compound 122
E8
Compound 149
E9
Compound 158
E10
Compound 159
E11
Compound 179
E12
Compound 187
E13
Compound 194
E14
Compound 223
E15
Compound 243
E16
Compound 262
E17
Compound 278
E18
Compound 286
E19
Compound 287
E20
Compound 289
E21
Compound 295
E22
Compound 316
E23
Compound 342
E24
Compound 369
E25
Compound 405
E26
Compound 439
E27
Compound 449
E28
Compound 493
E29
Compound 541
E30
Compound 563
E31
Compound 567
E32
Compound 586
E33
Compound 619
E34
Compound 1277
E35
Compound 703
E36
Compound 705
E37
Compound 762
E38
Compound 766
E39
Compound 767
E40
Compound 771
E41
Compound 785
E42
Compound 837
E43
Compound 897
E44
Compound 976
E45
Compound 991
E46
Compound 1061
E47
Compound 1070
E48
Compound 1117
E49
Compound 1175
E50
Compound 1228
E51
Compound 1232
E52
Compound 1233
E53
Compound 1237
E54
Compound 1289
E61
Compound 1308
E62
Compound 1294
E63

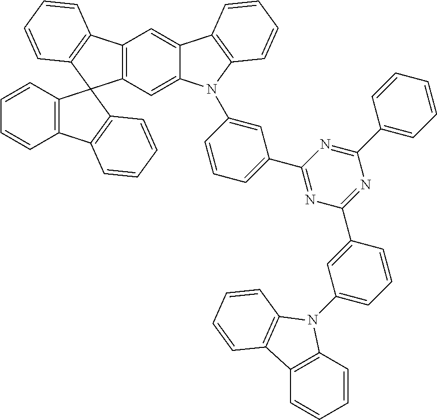 [SMILES: c1ccc(-c2nc(-c3cccc(-n4c5ccccc5c5ccccc54)c3)nc(-c3cccc(-n4c5ccccc5c5cc6c(cc54)C4(c5ccccc5-c5ccccc54)c4ccccc4-6)c3)n2)cc1]

E64 (1301)

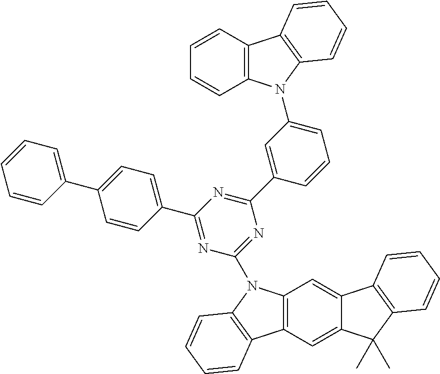 [SMILES: CC1(C)c2ccccc2-c2cc3c(cc21)c1ccccc1n3-c1nc(-c2ccc(-c3ccccc3)cc2)nc(-c2cccc(-n3c4ccccc4c4ccccc43)c2)n1]

E65 (1379)

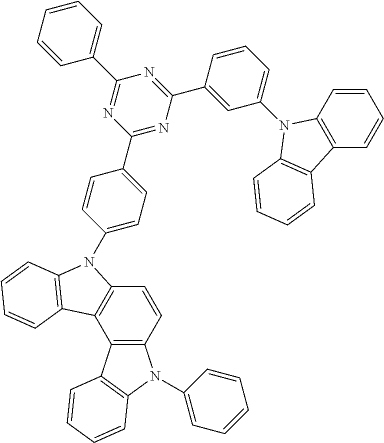 [SMILES: c1ccc(-c2nc(-c3ccc(-n4c5ccccc5c5c6c7ccccc7n(-c7ccccc7)c6ccc54)cc3)nc(-c3cccc(-n4c5ccccc5c5ccccc54)c3)n2)cc1]

E66 (1317)

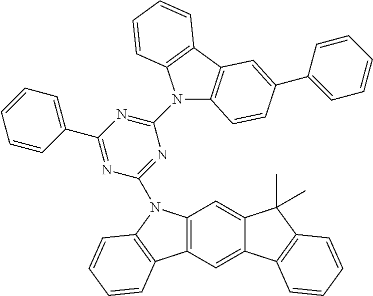 [SMILES: CC1(C)c2ccccc2-c2cc3c4ccccc4n(-c4nc(-c5ccccc5)nc(-n5c6ccccc6c6cc(-c7ccccc7)ccc65)n4)c3cc21]

E67 (1392)

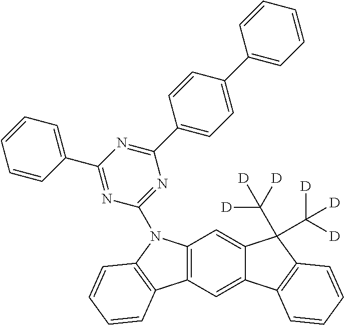 [SMILES: [2H]C([2H])([2H])C1(C([2H])([2H])[2H])c2ccccc2-c2cc3c4ccccc4n(-c4nc(-c5ccccc5)nc(-c5ccc(-c6ccccc6)cc5)n4)c3cc21]

E68 (1393)

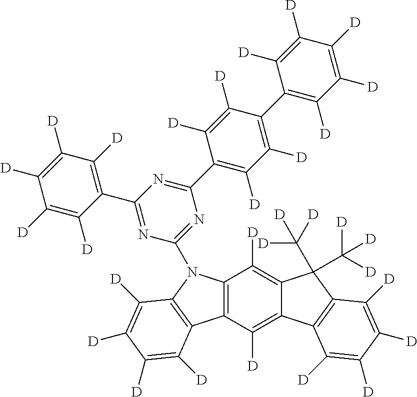 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c5c(c([2H])c43)C(C([2H])([2H])[2H])(C([2H])([2H])[2H])c3c([2H])c([2H])c([2H])c([2H])c3-5)n2)c([2H])c1[2H]]

E69 (1394).

The preparation of the compounds of the formula (1) or of the preferred compounds from table 1 and of the compounds E1 to E54 and E60 to E69 is known to those skilled in the art. The compounds can be prepared by synthesis steps known to those skilled in the art, for example bromination, Suzuki coupling, Ullmann coupling, Hartwig-Buchwald coupling, etc. A suitable synthesis method is shown in general terms in scheme 1 below, where the symbols and indices used have the definitions given above and L is phenylene.

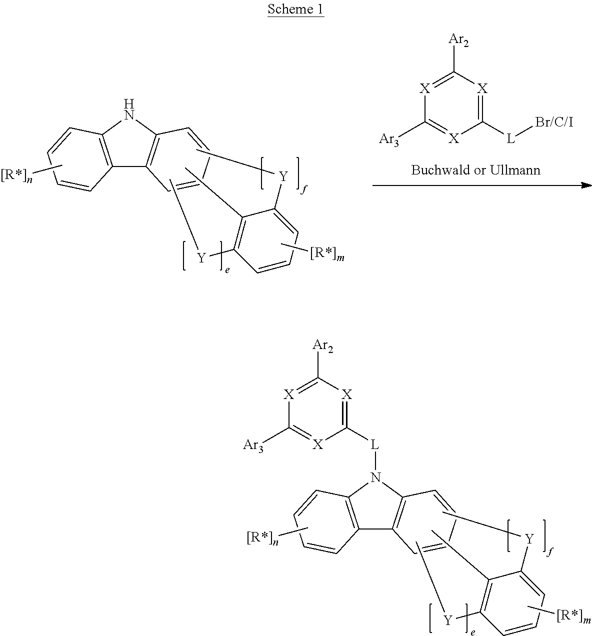

A suitable synthesis method is shown in general terms in scheme 2 below, where the symbols and indices used have the definitions given above and L is a single bond.

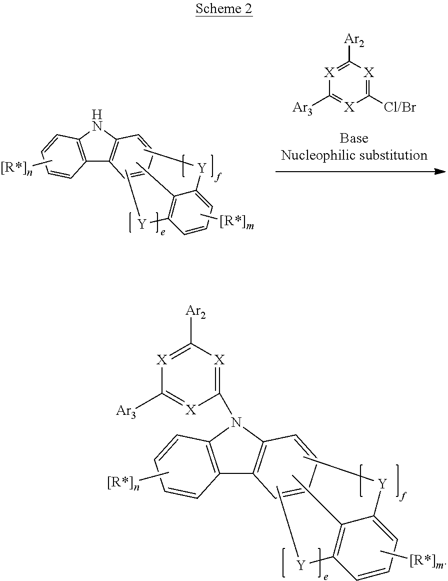

There follows a description of the host material 2 and its preferred embodiments that is/are present in the device of the invention. The preferred embodiments of the host material 2 of the formula (2) are also applicable to the mixture and/or formulation of the invention.

Host material 2 is at least one compound of the formula (2)

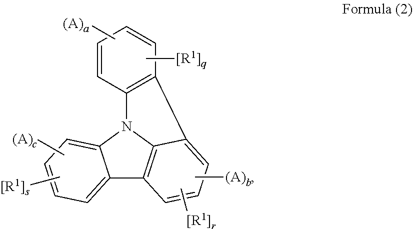

where the symbols and indices used are as follows:
A at each instance is independently a group of the formula (3) or (4),

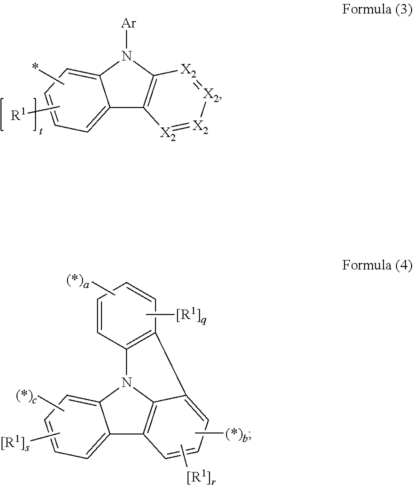

X₂ is the same or different at each instance and is CH, CR¹ or N, where not more than 2 symbols X₂ can be N;
* indicates the binding site to the formula (2);
R¹ is the same or different at each instance and is selected from the group consisting of CN, a straight-chain alkyl, alkoxy or thioalkyl group having 1 to 20 carbon atoms or a branched or cyclic alkyl, alkoxy or thioalkyl group having 3 to 20 carbon atoms, an aromatic or heteroaromatic ring system having 5 to 40 ring atoms, an aryloxy or heteroaryloxy group having 5 to 40 ring atoms, or an aralkyl or heteroaralkyl group having 5 to 40 ring atoms; at the same time, it is possible for two substituents R¹ bonded to the same carbon atom or to adjacent carbon atoms to form a monocyclic or polycyclic, aliphatic, aromatic or heteroaromatic ring system that may be substituted by one or more R² radicals;
Ar at each instance is in each case independently an aryl group which has 6 to 40 ring atoms and may be substituted by one or more R # radicals, or a heteroaryl group which has 5 to 40 ring atoms and may be substituted by one or more R # radicals;
R # is the same or different at each instance and is selected from the group consisting of D, F, Cl, Br, I, CN, NO₂, C(═O)R², P(═O)(Ar₁)₂, P(Ar₁)₂, B(Ar₁)₂, Si(Ar₁)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR² and where one or more hydrogen atoms may be replaced by D, F, Cl, Br, I, CN or NO₂, an aromatic or heteroaromatic ring system which has 5 to 40 ring atoms and may be substituted in each case by one or more R² radicals, an aryloxy or heteroaryloxy group which has 5 to 40 ring atoms and may be substituted by one or more R² radicals, or an aralkyl or heteroaralkyl group which has 5 to 40 ring atoms and may be substituted by one or more R² radicals;
R² is the same or different at each instance and is selected from the group consisting of H, D, F, Cl, Br, I, CN, NO₂, N(Ar₁)₂, NH₂, N(R³)₂, C(═O)Ar₁, C(═O)H, C(═O)R³, P(═O)(Ar₁)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R³ radicals, where one or more nonadjacent CH₂ groups may be replaced by HC═CH, R³C═CR³, C≡C, Si(R³)₂, Ge(R³)₂, Sn(R³)₂, C═O, C═S, C═Se, C═NR³, P(═O)(R³), SO, SO₂, NH, NR³, O, S, CONH or CONR³ and where one or more hydrogen atoms may be replaced by D, F, Cl, Br, I, CN or NO₂, an aromatic or heteroaromatic ring system that has 5 to 60 ring atoms and may be substituted in each case by one or more R³ radicals, an aryloxy or heteroaryloxy group that has 5 to 60 ring atoms and may be substituted by one or more R³ radicals, or a combination of these systems, where optionally two or more adjacent substituents R² may form a monocyclic or polycyclic, aliphatic, aromatic or heteroaromatic ring system that may be substituted by one or more R³ radicals;
R³ is the same or different at each instance and is selected from the group consisting of H, D, F, CN, an aliphatic hydrocarbyl radical having 1 to 20 carbon atoms, or an aromatic or heteroaromatic ring system having 5 to 30 ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents together to form a mono- or polycyclic, aliphatic ring system;
Ar₁ is the same or different at each instance and is an aromatic or heteroaromatic ring system which has 5 to 30 ring atoms and may be substituted by one or more nonaromatic R³ radicals; at the same time, two Ar₁ radicals bonded to the same nitrogen atom, phosphorus atom or boron atom may also be bridged to one another by a single bond or a bridge selected from N(R³), C(R³)₂, O or S;
a, b, c at each instance are each independently 0 or 1, where the sum total of the indices at each instance a+b+c is 1; and
q, r, s, t at each instance are each independently 0 or 1.

In one embodiment of the invention, for the device of the invention, compounds of the formula (2) as described above are selected, which are used in the light-emitting layer with compounds of the formula (1) as described above or described as preferred, or with the compounds from table 1 or the compounds E1 to E54 and E60 to E69.

In compounds of the formula (2), a, b, c at each instance are each independently 0 or 1, where the sum total of the indices at each instance a+b+c is 1. c is preferably defined as 1.

Compounds of the formula (2) may be represented by the following formulae (2a), (2b) and (2c):

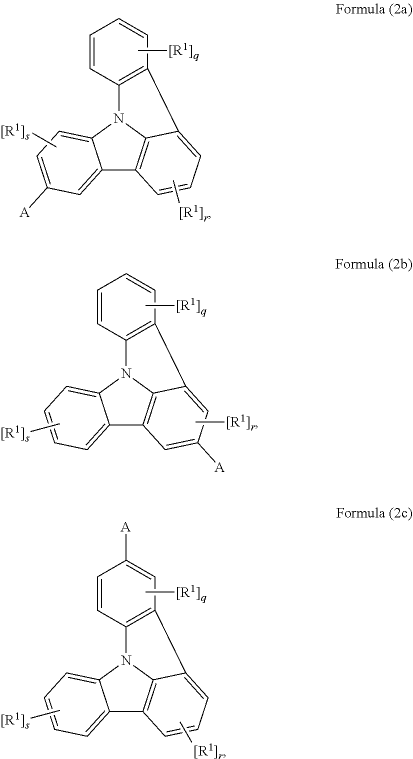

where A, R¹, q, r, s and t have a definition given above or given hereinafter. Preference is given here to compounds of the formula (2a).

The invention accordingly further provides an organic electroluminescent device as described above or described as preferred, wherein the host material 2 corresponds to a compound of the formula (2a), (2b) or (2c).

R¹ in compounds of the formula (2) and of the formulae (2a) to (2c) or preferred compounds of the formulae (2) and (2a) to (2c), as described above, is the same or different at each instance and is selected from the group consisting of CN, a straight-chain alkyl, alkoxy or thioalkyl group having 1 to 20 carbon atoms or a branched or cyclic alkyl, alkoxy or thioalkyl group having 3 to 20 carbon atoms, an aromatic or heteroaromatic ring system having 5 to 40 ring atoms, an aryloxy or heteroaryloxy group having 5 to 40 ring atoms, or an aralkyl or heteroaralkyl group having 5 to 40 ring atoms, at the same time, it is possible for two substituents R¹ bonded to the same carbon atom or to adjacent carbon atoms to form a monocyclic or polycyclic, aliphatic, aromatic or heteroaromatic ring system that may be substituted by one or more R² radicals.

If two or more R¹ radicals are bonded to adjacent carbon atoms, the monocyclic or polycyclic, aliphatic, aromatic or heteroaromatic ring system is preferably selected from the group of (S-1) to (S-4)

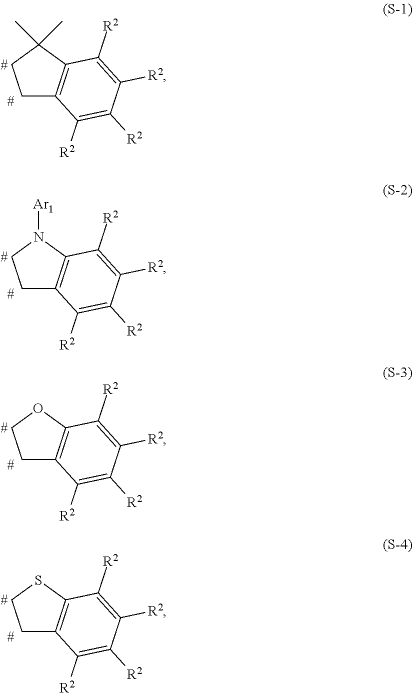

where Ar₁ and R² have a definition given above or definition given as preferred and # indicates the bonding sites to the rest of the respective structure, for example to adjacent positions identified by X₂ in compounds of the formulae (2), (2a), (2b) and (2c). Particular preference is given here to selecting (S-1) or (S-2).

R¹ in compounds of the formula (2) and of the formulae (2a) to (2c) or preferred compounds of the formulae (2) and (2a) to (2c), as described above, is the same or different at each instance and is preferably selected from the group consisting of CN, a straight-chain alkyl, alkoxy or thioalkyl group having 1 to 20 carbon atoms or a branched or cyclic alkyl, alkoxy or thioalkyl group having 3 to 20 carbon atoms, an aromatic or heteroaromatic ring system having 5 to 40 ring atoms, an aryloxy or heteroaryloxy group having 5 to 40 ring atoms, or an aralkyl or heteroaralkyl group having 5 to 40 ring atoms. The substituent R¹ at each instance is more preferably independently CN or an aryl group having 6 to 40 carbon atoms, as described above. R¹ at each instance is more preferably independently phenyl.

In compounds of the formulae (2), (2a), (2b) and (2c), the sum total of the indices q+r+s is preferably 0, 1 or 2, where R¹ has a definition given above. In compounds of the formulae (2), (2a), (2b) and (2c), the sum total of the indices q+r+s is preferably 0 or 1, where R¹ has a definition given above.

In compounds of the formulae (2), (2a), (2b) and (2c), q, r and s are preferably 0 or 1. Preferably, q is 1 if the sum total of the indices q+r+s is 1. Preferably, q, r and s are 0.

In formula (4)

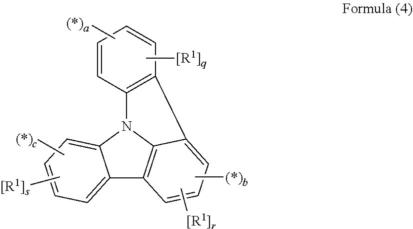

q, r and s are 0 or 1, where R¹ has a definition given above. Preferably, the sum total of the indices q+r+s in formula (4) is 0 or 1. In formula (4), q, r and s are more preferably 0.

In formula (3)

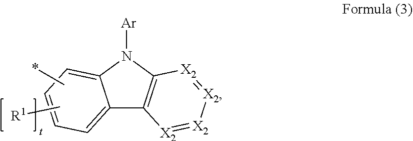

t is in each case independently preferably 0 or 1. In formula (3), t is preferably the same and is 0.

In compounds of the formulae (2), (2a), (2b) and (2c) or preferred compounds of the formulae (2), (2a), (2b) and (2c), X₂ is the same or different at each instance and is CH, CR¹ or N, where not more than 2 symbols X₂ can be N.

In compounds of the formulae (2), (2a), (2b) and (2c) or preferred compounds of the formulae (2), (2a), (2b) and (2c), X₂ is preferably the same or different at each instance and is CH, CR¹ or N, where not more than 1 symbol X₂ is N.

In compounds of the formulae (2), (2a), (2b) and (2c) or preferred compounds of the formulae (2), (2a), (2b) and (2c), X₂ is more preferably the same or different at each instance and is CH at two instances and CR¹ at two instances, or CH at three instances and CR¹ at one instance, where the substituents R¹ at each instance independently have a definition given above.

Ar at each instance is in each case independently an aryl group which has 6 to 40 ring atoms and may be substituted by one or more R # radicals, or a heteroaryl group which has 5 to 40 ring atoms and may be substituted by one or more R # radicals, where the R # radical has a definition given above or given with preference hereinafter.

Ar at each instance is preferably in each case independently an aryl group which has 6 to 40 ring atoms and may be substituted by one or more R # radicals, or a heteroaryl group having 5 to 40 ring atoms and containing O or S as heteroatom, which may be substituted by one or more R # radicals, where the R # radical has a definition given above or given with preference.

Ar at each instance is preferably an aryl group which has 6 to 18 carbon atoms and may be substituted by one or more R # radicals, or dibenzofuranyl or dibenzothiophenyl which may be substituted by one or more R # radicals, where the R # radical has a definition given above or given with preference hereinafter.

Ar is more preferably phenyl, dibenzofuran-substituted phenyl, dibenzothiophene-substituted phenyl, 1,3-biphenyl, 1,4-biphenyl, terphenyl, quaterphenyl, naphthyl, 9,9-dimethylfluorenyl, 9,9-diphenylfluorenyl, bispirofluorenyl, triphenylenyl, dibenzofuranyl, phenyl-substituted dibenzofuranyl, dibenzothiophenyl or phenyl-substituted dibenzothiophenyl.

Ar is most preferably phenyl, 1,3-biphenyl, 1,4-biphenyl, naphth-2-yl or triphenyl-2-yl.

In compounds of the formulae (2), (2a), (2b) and (2c) or preferred compounds of the formulae (2), (2a), (2b) and (2c), R # is the same or different at each instance and is preferably selected from the group consisting of D, CN and an aromatic or heteroaromatic ring system which has 5 to 40 ring atoms and may be substituted in each case by one or more R² radicals.

In compounds of the formulae (2), (2a), (2b) and (2c) or preferred compounds of the formulae (2), (2a), (2b) and (2c), R # is the same or different at each instance and is more preferably an unsubstituted aromatic ring system having 5 to 20 ring atoms, preferably phenyl.

In a preferred embodiment of the invention, A conforms to the formula (4) as described above or with substituents as described as preferred.

In a preferred embodiment of the invention, A conforms to the formula (3) as described above or with substituents as described as preferred.

Compounds of the formulae (2), (2a), (2b) and (2c) where A conforms to the formula (3) and q, r, s and t are 0 may be represented by the formulae (2d) and (2e)

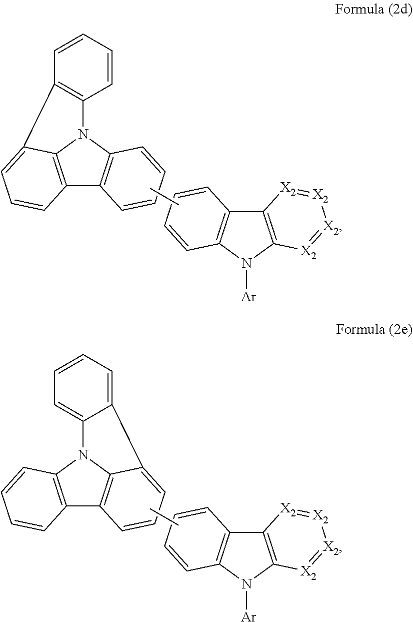

where X₂ and Ar have a definition given above or given as preferred.

The invention accordingly further provides an organic electroluminescent device as described above or described as preferred, wherein the at least one compound of the formula (2) corresponds to a compound of the formula (2d) or of the formula (2e).

In a preferred embodiment of the compounds of the formulae (2), (2a), (2b), (2c), (2d) or (2e), the substituents of the formulae (3) and (4) are each joined to one another in the 2 position or 5 position of the indolo[3,2,1-jk]carbazole, as shown in schematic form below, where the dotted line indicates the linkage to the substituents of the formulae (3) and (4):

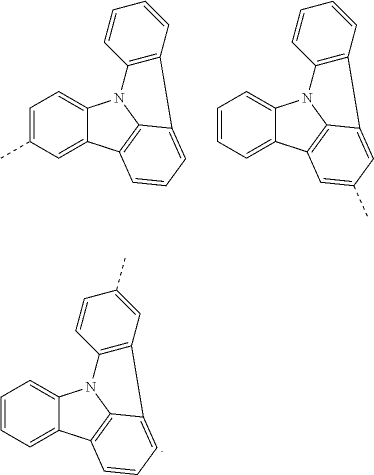

Examples of suitable host materials of the formulae (2), (2a), (2b), (2c), (2d) and (2e) that are selected in accordance with the invention and are preferably used in combination with at least one compound of the formula (1) in the electroluminescent device of the invention are the structures given below in table 3.

TABLE 3

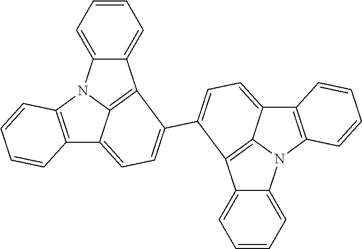 [SMILES: c1ccc2c(c1)c1ccc(-c3ccc4c5ccccc5n5c6ccccc6c3c45)c3c4ccccc4n2c13]

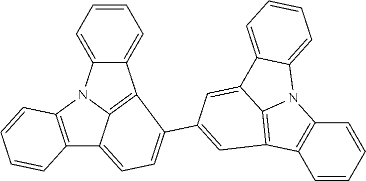 [SMILES: c1ccc2c(c1)c1cc(-c3ccc4c5ccccc5n5c6ccccc6c3c45)cc3c4ccccc4n2c13]

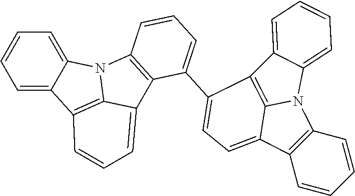 [SMILES: c1ccc2c(c1)c1cccc3c4c(-c5ccc6c7ccccc7n7c8ccccc8c5c67)cccc4n2c13]

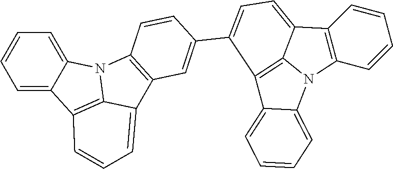 [SMILES: c1ccc2c(c1)c1cccc3c4cc(-c5ccc6c7ccccc7n7c8ccccc8c5c67)ccc4n2c13]

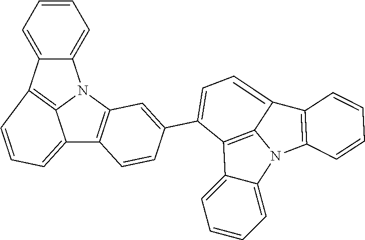 [SMILES: c1ccc2c(c1)c1cccc3c4ccc(-c5ccc6c7ccccc7n7c8ccccc8c5c67)cc4n2c13]

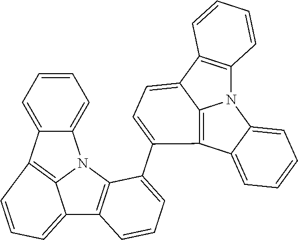 [SMILES: c1ccc2c(c1)c1ccc(-c3cccc4c5cccc6c7ccccc7n(c34)c65)c3c4ccccc4n2c13]

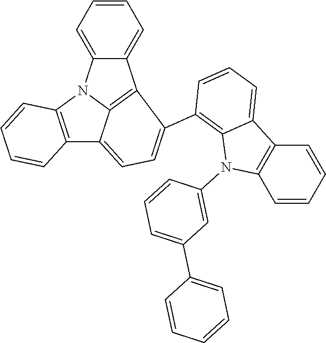 [SMILES: c1ccc(-c2cccc(-n3c4ccccc4c4cccc(-c5ccc6c7ccccc7n7c8ccccc8c5c67)c43)c2)cc1]

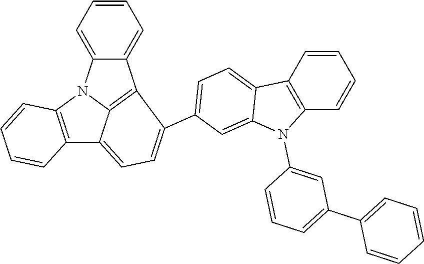 [SMILES: c1ccc(-c2cccc(-n3c4ccccc4c4ccc(-c5ccc6c7ccccc7n7c8ccccc8c5c67)cc43)c2)cc1]

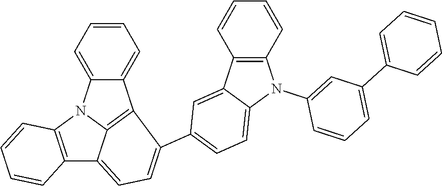 [SMILES: c1ccc(-c2cccc(-n3c4ccccc4c4cc(-c5ccc6c7ccccc7n7c8ccccc8c5c67)ccc43)c2)cc1]

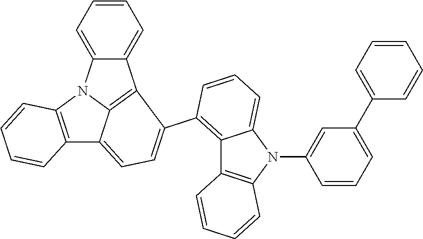 [SMILES: c1ccc(-c2cccc(-n3c4ccccc4c4c(-c5ccc6c7ccccc7n7c8ccccc8c5c67)cccc43)c2)cc1]

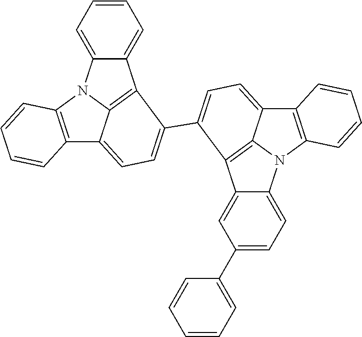 [SMILES: c1ccc(-c2ccc3c(c2)c2c(-c4ccc5c6ccccc6n6c7ccccc7c4c56)ccc4c5ccccc5n3c42)cc1]

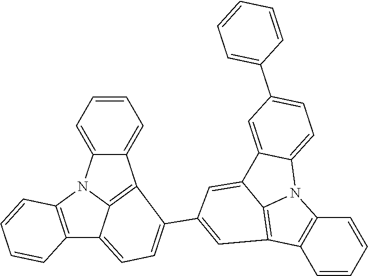 [SMILES: c1ccc(-c2ccc3c(c2)c2cc(-c4ccc5c6ccccc6n6c7ccccc7c4c56)cc4c5ccccc5n3c42)cc1]

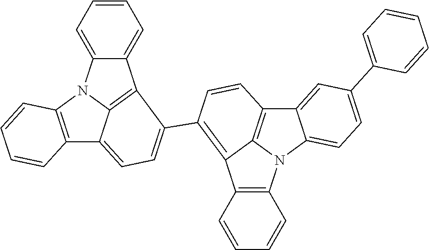 [SMILES: c1ccc(-c2ccc3c(c2)c2ccc(-c4ccc5c6ccccc6n6c7ccccc7c4c56)c4c5ccccc5n3c24)cc1]

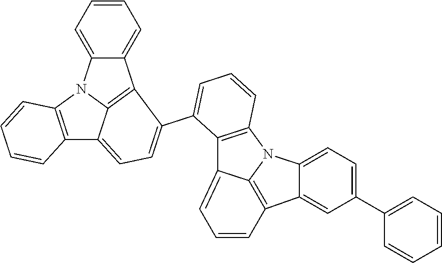 [SMILES: c1ccc(-c2ccc3c(c2)c2cccc4c5c(-c6ccc7c8ccccc8n8c9ccccc9c6c78)cccc5n3c24)cc1]

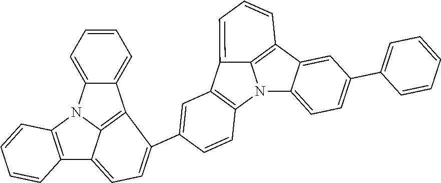 [SMILES: c1ccc(-c2ccc3c(c2)c2cccc4c5cc(-c6ccc7c8ccccc8n8c9ccccc9c6c78)ccc5n3c24)cc1]

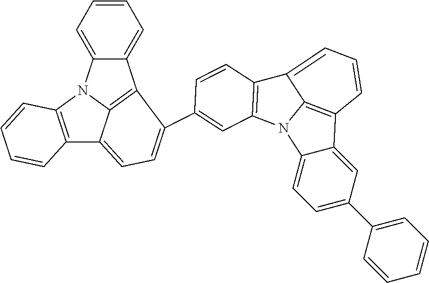 [SMILES: c1ccc(-c2ccc3c(c2)c2cccc4c5ccc(-c6ccc7c8ccccc8n8c9ccccc9c6c78)cc5n3c24)cc1]

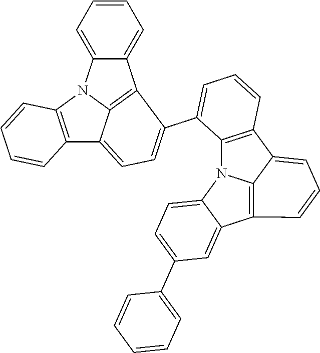 [SMILES: c1ccc(-c2ccc3c(c2)c2cccc4c5cccc(-c6ccc7c8ccccc8n8c9ccccc9c6c78)c5n3c24)cc1]

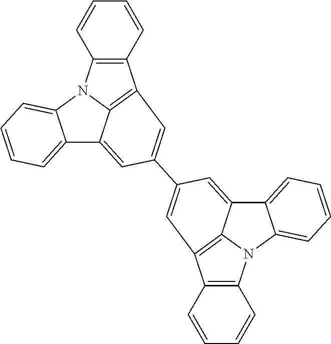 [SMILES: c1ccc2c(c1)c1cc(-c3cc4c5ccccc5n5c6ccccc6c(c3)c45)cc3c4ccccc4n2c13]

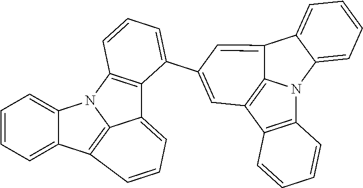 [SMILES: c1ccc2c(c1)c1cc(-c3cccc4c3c3cccc5c6ccccc6n4c53)cc3c4ccccc4n2c13]

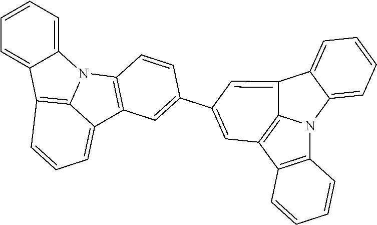 [SMILES: c1ccc2c(c1)c1cccc3c4cc(-c5cc6c7ccccc7n7c8ccccc8c(c5)c67)ccc4n2c13]

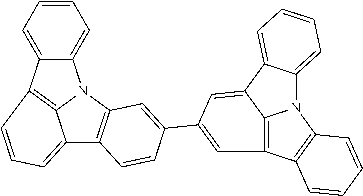 [SMILES: c1ccc2c(c1)c1cc(-c3ccc4c5cccc6c7ccccc7n(c4c3)c65)cc3c4ccccc4n2c13]

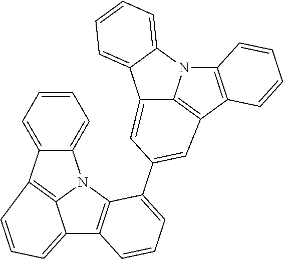 [SMILES: c1ccc2c(c1)c1cc(-c3cccc4c5cccc6c7ccccc7n(c34)c65)cc3c4ccccc4n2c13]

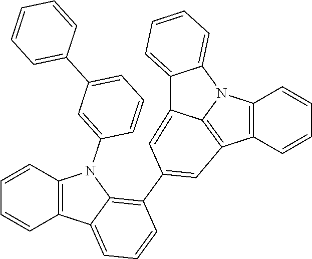 [SMILES: c1ccc(-c2cccc(-n3c4ccccc4c4cccc(-c5cc6c7ccccc7n7c8ccccc8c(c5)c67)c43)c2)cc1]

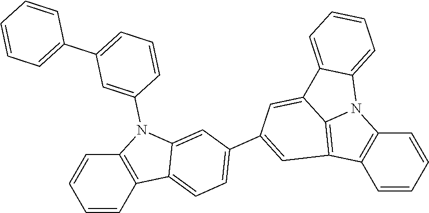 [SMILES: c1ccc(-c2cccc(-n3c4ccccc4c4ccc(-c5cc6c7ccccc7n7c8ccccc8c(c5)c67)cc43)c2)cc1]

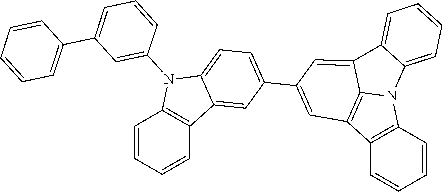 [SMILES: c1ccc(-c2cccc(-n3c4ccccc4c4cc(-c5cc6c7ccccc7n7c8ccccc8c(c5)c67)ccc43)c2)cc1]

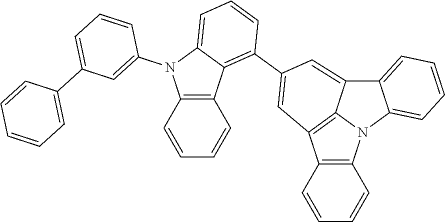 [SMILES: c1ccc(-c2cccc(-n3c4ccccc4c4c(-c5cc6c7ccccc7n7c8ccccc8c(c5)c67)cccc43)c2)cc1]

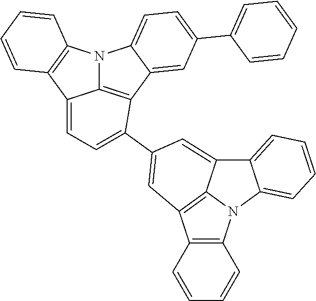 [SMILES: c1ccc(-c2ccc3c(c2)c2c(-c4cc5c6ccccc6n6c7ccccc7c(c4)c56)ccc4c5ccccc5n3c42)cc1]

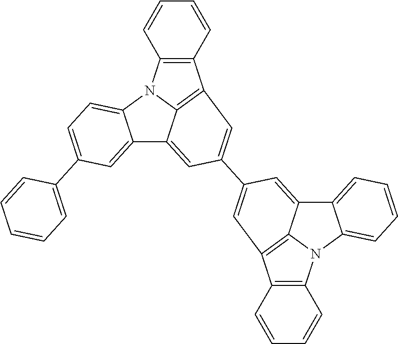 [SMILES: c1ccc(-c2ccc3c(c2)c2cc(-c4cc5c6ccccc6n6c7ccccc7c(c4)c56)cc4c5ccccc5n3c42)cc1]

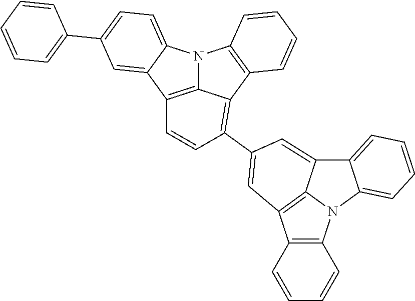 [SMILES: c1ccc(-c2ccc3c(c2)c2ccc(-c4cc5c6ccccc6n6c7ccccc7c(c4)c56)c4c5ccccc5n3c24)cc1]

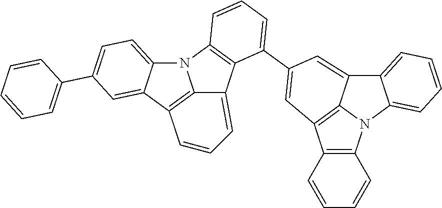 [SMILES: c1ccc(-c2ccc3c(c2)c2cccc4c5c(-c6cc7c8ccccc8n8c9ccccc9c(c6)c78)cccc5n3c24)cc1]

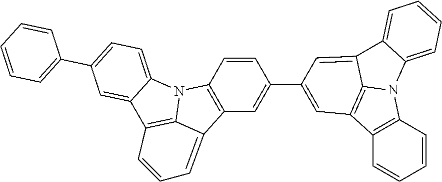 [SMILES: c1ccc(-c2ccc3c(c2)c2cccc4c5cc(-c6cc7c8ccccc8n8c9ccccc9c(c6)c78)ccc5n3c24)cc1]

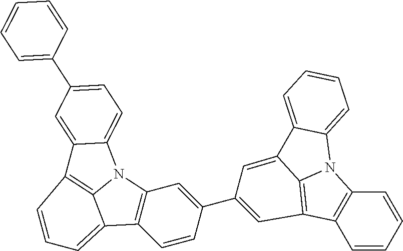 [SMILES: c1ccc(-c2ccc3c(c2)c2cccc4c5ccc(-c6cc7c8ccccc8n8c9ccccc9c(c6)c78)cc5n3c24)cc1]

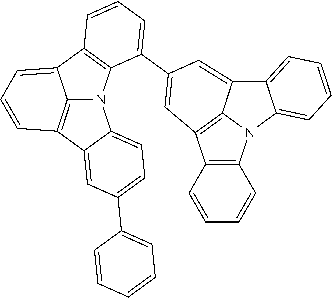 [SMILES: c1ccc(-c2ccc3c(c2)c2cccc4c5cccc(-c6cc7c8ccccc8n8c9ccccc9c(c6)c78)c5n3c24)cc1]

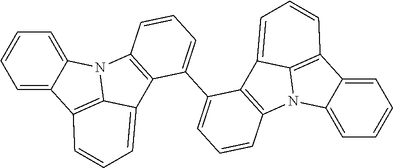 [SMILES: c1ccc2c(c1)c1cccc3c4c(-c5cccc6c5c5cccc7c8ccccc8n6c75)cccc4n2c13]

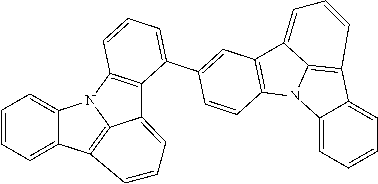 [SMILES: c1ccc2c(c1)c1cccc3c4cc(-c5cccc6c5c5cccc7c8ccccc8n6c75)ccc4n2c13]

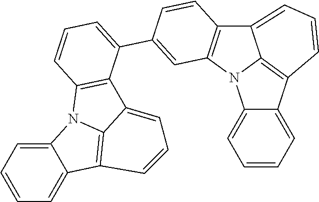 [SMILES: c1ccc2c(c1)c1cccc3c4ccc(-c5cccc6c5c5cccc7c8ccccc8n6c75)cc4n2c13]

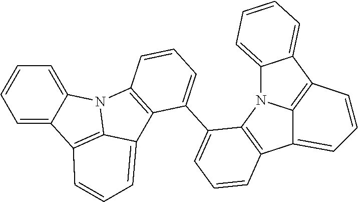 [SMILES: c1ccc2c(c1)c1cccc3c4c(-c5cccc6c7cccc8c9ccccc9n(c56)c87)cccc4n2c13]

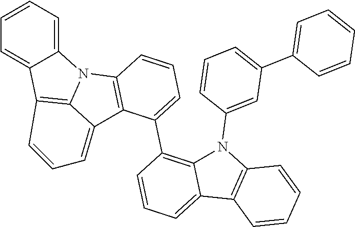 [SMILES: c1ccc(-c2cccc(-n3c4ccccc4c4cccc(-c5cccc6c5c5cccc7c8ccccc8n6c75)c43)c2)cc1]

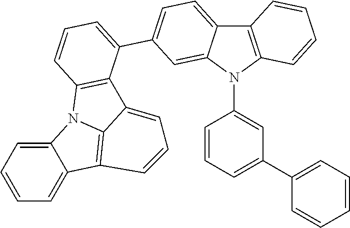 [SMILES: c1ccc(-c2cccc(-n3c4ccccc4c4ccc(-c5cccc6c5c5cccc7c8ccccc8n6c75)cc43)c2)cc1]

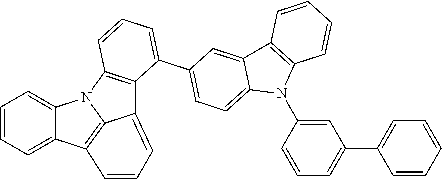 [SMILES: c1ccc(-c2cccc(-n3c4ccccc4c4cc(-c5cccc6c5c5cccc7c8ccccc8n6c75)ccc43)c2)cc1]

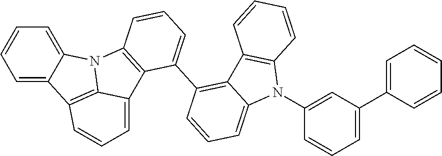 [SMILES: c1ccc(-c2cccc(-n3c4ccccc4c4c(-c5cccc6c5c5cccc7c8ccccc8n6c75)cccc43)c2)cc1]

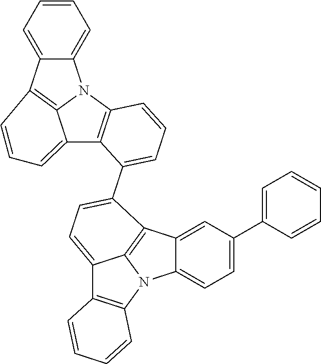 [SMILES: c1ccc(-c2ccc3c(c2)c2c(-c4cccc5c4c4cccc6c7ccccc7n5c64)ccc4c5ccccc5n3c42)cc1]

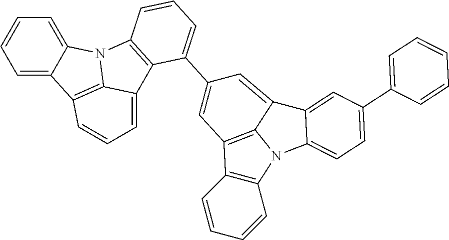 [SMILES: c1ccc(-c2ccc3c(c2)c2cc(-c4cccc5c4c4cccc6c7ccccc7n5c64)cc4c5ccccc5n3c42)cc1]

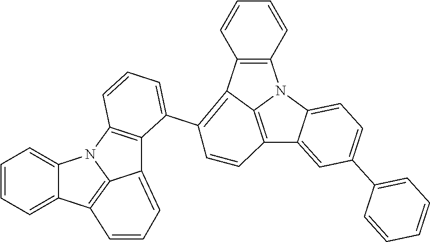 [SMILES: c1ccc(-c2ccc3c(c2)c2ccc(-c4cccc5c4c4cccc6c7ccccc7n5c64)c4c5ccccc5n3c24)cc1]

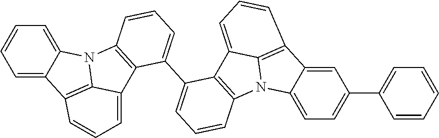 [SMILES: c1ccc(-c2ccc3c(c2)c2cccc4c5c(-c6cccc7c6c6cccc8c9ccccc9n7c86)cccc5n3c24)cc1]

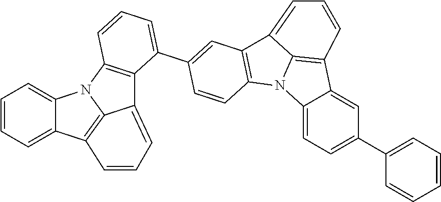 [SMILES: c1ccc(-c2ccc3c(c2)c2cccc4c5cc(-c6cccc7c6c6cccc8c9ccccc9n7c86)ccc5n3c24)cc1]

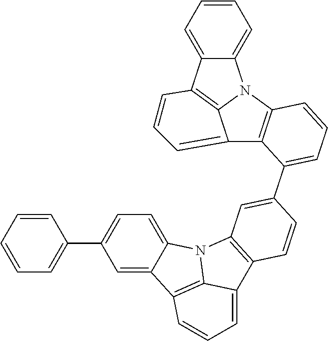 [SMILES: c1ccc(-c2ccc3c(c2)c2cccc4c5ccc(-c6cccc7c6c6cccc8c9ccccc9n7c86)cc5n3c24)cc1]

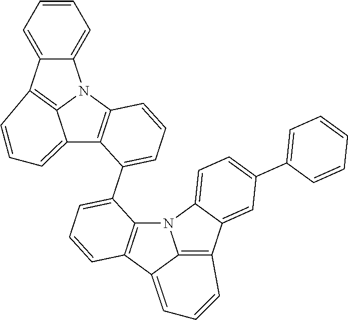 [SMILES: c1ccc(-c2ccc3c(c2)c2cccc4c5cccc(-c6cccc7c6c6cccc8c9ccccc9n7c86)c5n3c24)cc1]

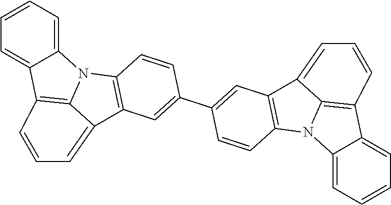 [SMILES: c1ccc2c(c1)c1cccc3c4cc(-c5ccc6c(c5)c5cccc7c8ccccc8n6c75)ccc4n2c13]

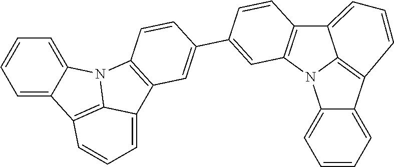 [SMILES: c1ccc2c(c1)c1cccc3c4cc(-c5ccc6c7cccc8c9ccccc9n(c6c5)c87)ccc4n2c13]

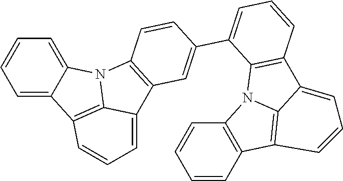 [SMILES: c1ccc2c(c1)c1cccc3c4cc(-c5cccc6c7cccc8c9ccccc9n(c56)c87)ccc4n2c13]

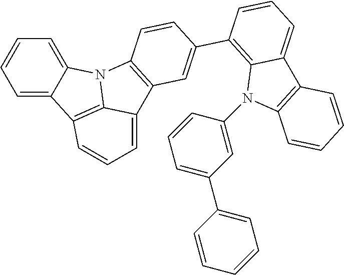 [SMILES: c1ccc(-c2cccc(-n3c4ccccc4c4cccc(-c5ccc6c(c5)c5cccc7c8ccccc8n6c75)c43)c2)cc1]

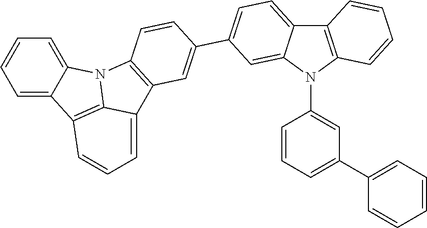 [SMILES: c1ccc(-c2cccc(-n3c4ccccc4c4ccc(-c5ccc6c(c5)c5cccc7c8ccccc8n6c75)cc43)c2)cc1]

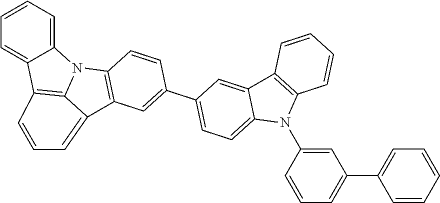 [SMILES: c1ccc(-c2cccc(-n3c4ccccc4c4cc(-c5ccc6c(c5)c5cccc7c8ccccc8n6c75)ccc43)c2)cc1]

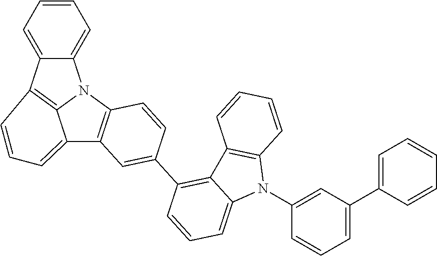 [SMILES: c1ccc(-c2cccc(-n3c4ccccc4c4c(-c5ccc6c(c5)c5cccc7c8ccccc8n6c75)cccc43)c2)cc1]

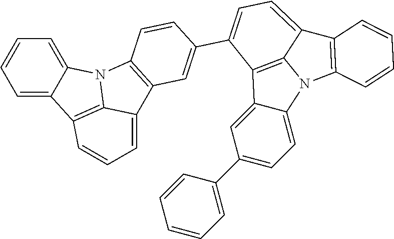 [SMILES: c1ccc(-c2ccc3c(c2)c2c(-c4ccc5c(c4)c4cccc6c7ccccc7n5c64)ccc4c5ccccc5n3c42)cc1]

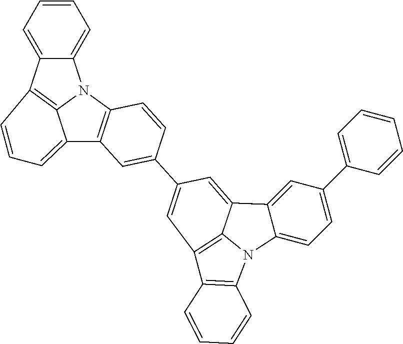 [SMILES: c1ccc(-c2ccc3c(c2)c2cc(-c4ccc5c(c4)c4cccc6c7ccccc7n5c64)cc4c5ccccc5n3c42)cc1]

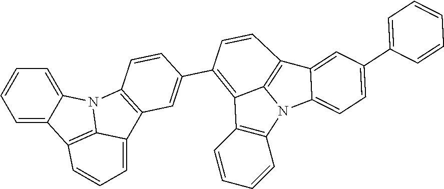 [SMILES: c1ccc(-c2ccc3c(c2)c2ccc(-c4ccc5c(c4)c4cccc6c7ccccc7n5c64)c4c5ccccc5n3c24)cc1]

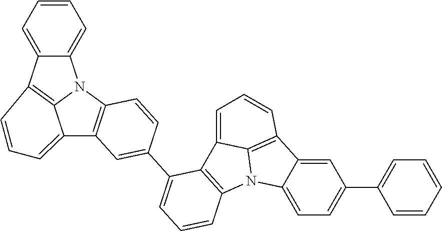 [SMILES: c1ccc(-c2ccc3c(c2)c2cccc4c5c(-c6ccc7c(c6)c6cccc8c9ccccc9n7c86)cccc5n3c24)cc1]

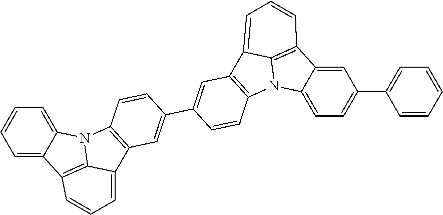 [SMILES: c1ccc(-c2ccc3c(c2)c2cccc4c5cc(-c6ccc7c(c6)c6cccc8c9ccccc9n7c86)ccc5n3c24)cc1]

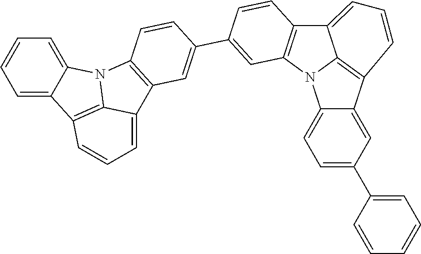 [SMILES: c1ccc(-c2ccc3c(c2)c2cccc4c5ccc(-c6ccc7c(c6)c6cccc8c9ccccc9n7c86)cc5n3c24)cc1]

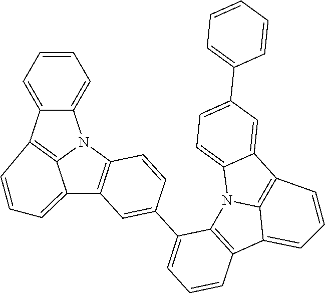 [SMILES: c1ccc(-c2ccc3c(c2)c2cccc4c5cccc(-c6ccc7c(c6)c6cccc8c9ccccc9n7c86)c5n3c24)cc1]

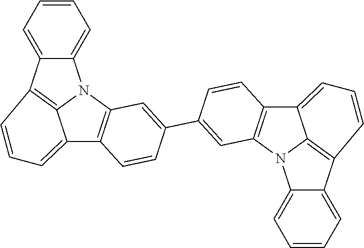 [SMILES: c1ccc2c(c1)c1cccc3c4ccc(-c5ccc6c7cccc8c9ccccc9n(c6c5)c87)cc4n2c13]

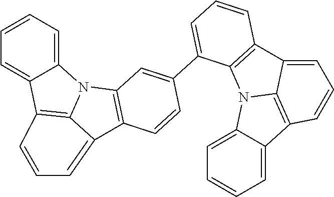 [SMILES: c1ccc2c(c1)c1cccc3c4ccc(-c5cccc6c7cccc8c9ccccc9n(c56)c87)cc4n2c13]

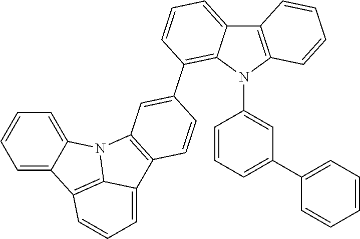 [SMILES: c1ccc(-c2cccc(-n3c4ccccc4c4cccc(-c5ccc6c7cccc8c9ccccc9n(c6c5)c87)c43)c2)cc1]

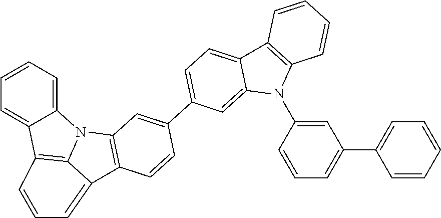 [SMILES: c1ccc(-c2cccc(-n3c4ccccc4c4ccc(-c5ccc6c7cccc8c9ccccc9n(c6c5)c87)cc43)c2)cc1]

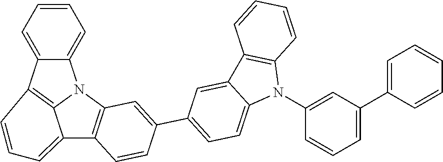 [SMILES: c1ccc(-c2cccc(-n3c4ccccc4c4cc(-c5ccc6c7cccc8c9ccccc9n(c6c5)c87)ccc43)c2)cc1]

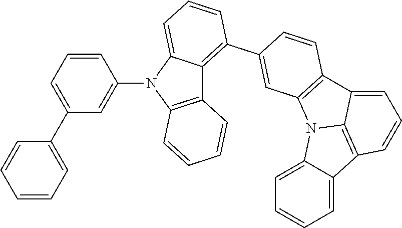 [SMILES: c1ccc(-c2cccc(-n3c4ccccc4c4c(-c5ccc6c7cccc8c9ccccc9n(c6c5)c87)cccc43)c2)cc1]

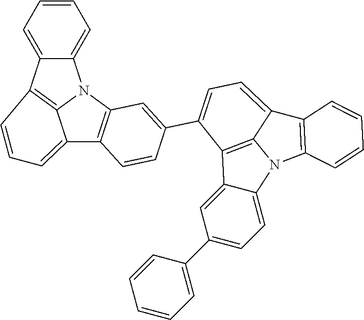 [SMILES: c1ccc(-c2ccc3c(c2)c2c(-c4ccc5c6cccc7c8ccccc8n(c5c4)c76)ccc4c5ccccc5n3c42)cc1]

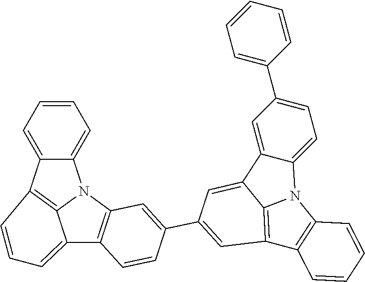 [SMILES: c1ccc(-c2ccc3c(c2)c2cc(-c4ccc5c6cccc7c8ccccc8n(c5c4)c76)cc4c5ccccc5n3c42)cc1]

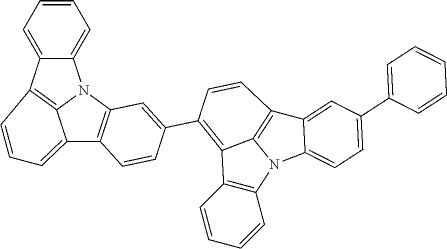 [SMILES: c1ccc(-c2ccc3c(c2)c2ccc(-c4ccc5c6cccc7c8ccccc8n(c5c4)c76)c4c5ccccc5n3c24)cc1]

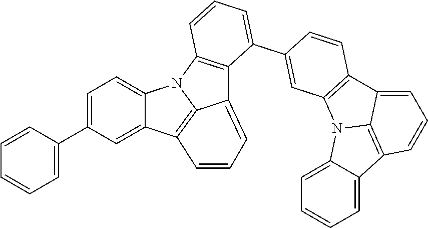 [SMILES: c1ccc(-c2ccc3c(c2)c2cccc4c5c(-c6ccc7c8cccc9c%10ccccc%10n(c7c6)c98)cccc5n3c24)cc1]

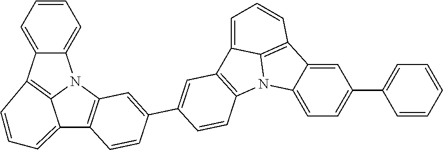 [SMILES: c1ccc(-c2ccc3c(c2)c2cccc4c5cc(-c6ccc7c8cccc9c%10ccccc%10n(c7c6)c98)ccc5n3c24)cc1]

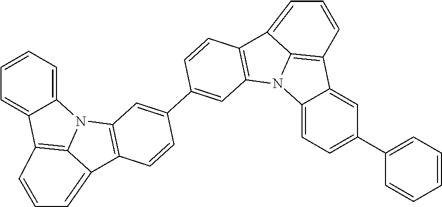 [SMILES: c1ccc(-c2ccc3c(c2)c2cccc4c5ccc(-c6ccc7c8cccc9c%10ccccc%10n(c7c6)c98)cc5n3c24)cc1]

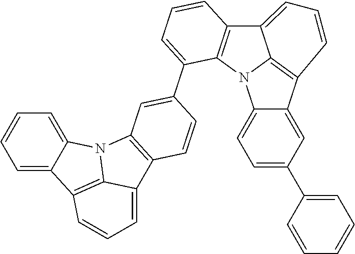 [SMILES: c1ccc(-c2ccc3c(c2)c2cccc4c5cccc(-c6ccc7c8cccc9c%10ccccc%10n(c7c6)c98)c5n3c24)cc1]

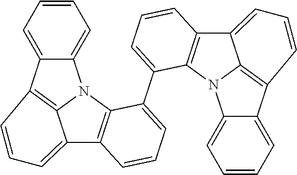 [SMILES: c1ccc2c(c1)c1cccc3c4cccc(-c5cccc6c7cccc8c9ccccc9n(c56)c87)c4n2c13]

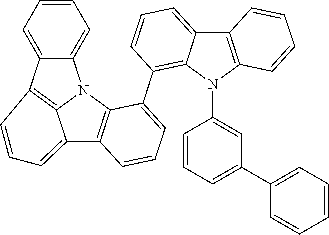 [SMILES: c1ccc(-c2cccc(-n3c4ccccc4c4cccc(-c5cccc6c7cccc8c9ccccc9n(c56)c87)c43)c2)cc1]

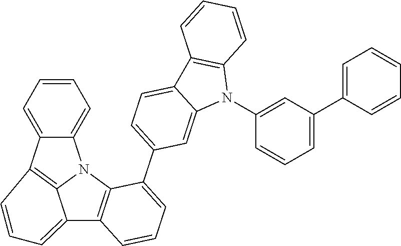 [SMILES: c1ccc(-c2cccc(-n3c4ccccc4c4ccc(-c5cccc6c7cccc8c9ccccc9n(c56)c87)cc43)c2)cc1]

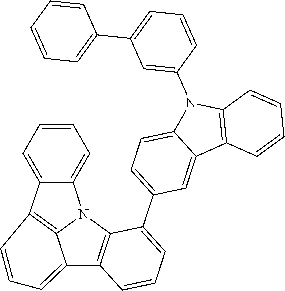 [SMILES: c1ccc(-c2cccc(-n3c4ccccc4c4cc(-c5cccc6c7cccc8c9ccccc9n(c56)c87)ccc43)c2)cc1]

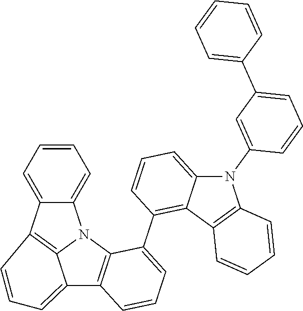 [SMILES: c1ccc(-c2cccc(-n3c4ccccc4c4c(-c5cccc6c7cccc8c9ccccc9n(c56)c87)cccc43)c2)cc1]

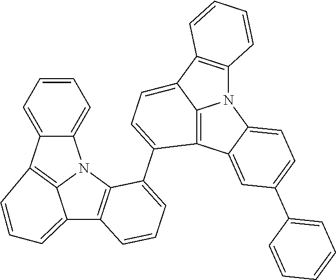 [SMILES: c1ccc(-c2ccc3c(c2)c2c(-c4cccc5c6cccc7c8ccccc8n(c45)c76)ccc4c5ccccc5n3c42)cc1]

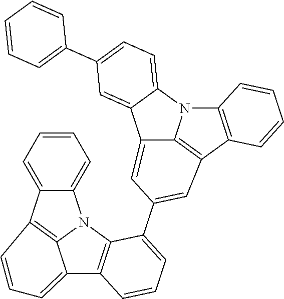 [SMILES: c1ccc(-c2ccc3c(c2)c2cc(-c4cccc5c6cccc7c8ccccc8n(c45)c76)cc4c5ccccc5n3c42)cc1]

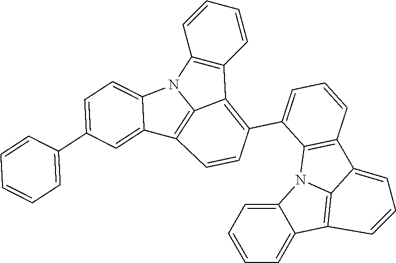 [SMILES: c1ccc(-c2ccc3c(c2)c2ccc(-c4cccc5c6cccc7c8ccccc8n(c45)c76)c4c5ccccc5n3c24)cc1]

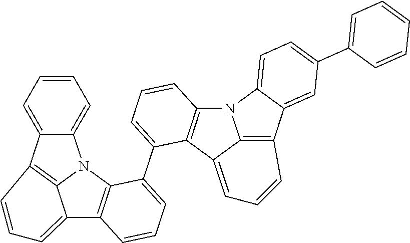 [SMILES: c1ccc(-c2ccc3c(c2)c2cccc4c5c(-c6cccc7c8cccc9c%10ccccc%10n(c67)c98)cccc5n3c24)cc1]

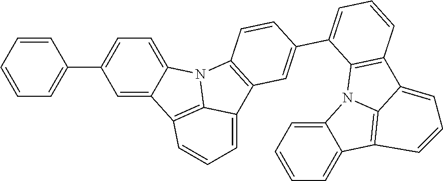 [SMILES: c1ccc(-c2ccc3c(c2)c2cccc4c5cc(-c6cccc7c8cccc9c%10ccccc%10n(c67)c98)ccc5n3c24)cc1]

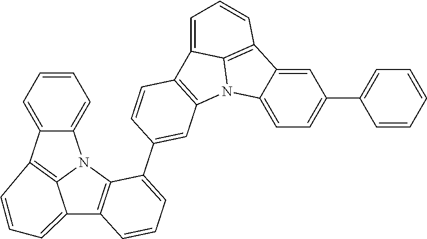 [SMILES: c1ccc(-c2ccc3c(c2)c2cccc4c5ccc(-c6cccc7c8cccc9c%10ccccc%10n(c67)c98)cc5n3c24)cc1]

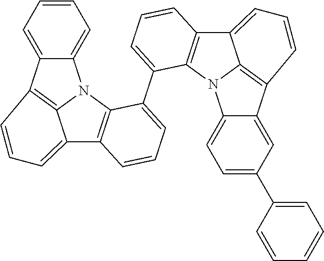 [SMILES: c1ccc(-c2ccc3c(c2)c2cccc4c5cccc(-c6cccc7c8cccc9c%10ccccc%10n(c67)c98)c5n3c24)cc1]

Particularly suitable compounds of the formula (2) that are preferably used in combination with at least one compound of the formula (1) in the electroluminescent device of the invention are the compounds H1 to H21 of table 4.

TABLE 4

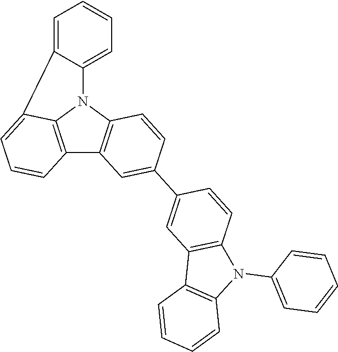 [SMILES: c1ccc(-n2c3ccccc3c3cc(-c4ccc5c(c4)c4cccc6c7ccccc7n5c64)ccc32)cc1]

H1

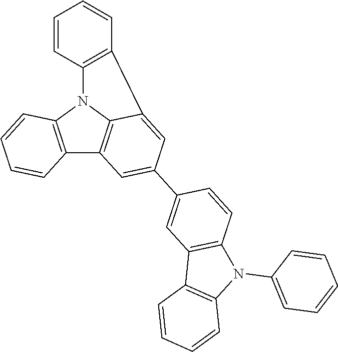 [SMILES: c1ccc(-n2c3ccccc3c3cc(-c4cc5c6ccccc6n6c7ccccc7c(c4)c56)ccc32)cc1]

H2

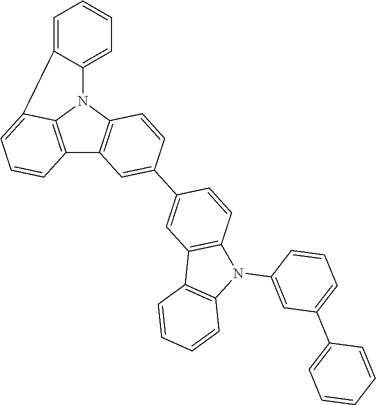 [SMILES: c1ccc(-c2cccc(-n3c4ccccc4c4cc(-c5ccc6c(c5)c5cccc7c8ccccc8n6c75)ccc43)c2)cc1]

H3

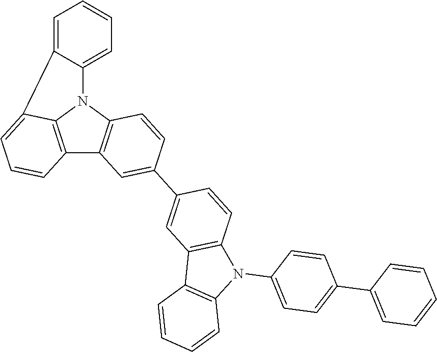 [SMILES: c1ccc(-c2ccc(-n3c4ccccc4c4cc(-c5ccc6c(c5)c5cccc7c8ccccc8n6c75)ccc43)cc2)cc1]

H4

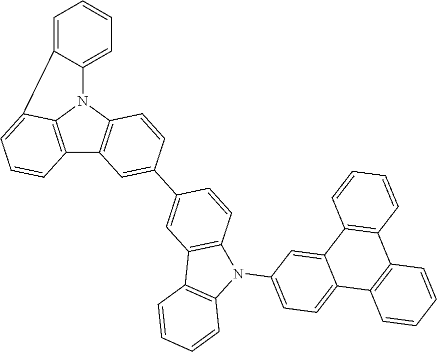 [SMILES: c1ccc2c(c1)c1ccccc1c1cc(-n3c4ccccc4c4cc(-c5ccc6c(c5)c5cccc7c8ccccc8n6c75)ccc43)ccc21]

H5

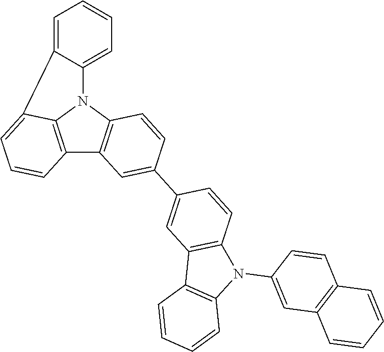 [SMILES: c1ccc2cc(-n3c4ccccc4c4cc(-c5ccc6c(c5)c5cccc7c8ccccc8n6c75)ccc43)ccc2c1]

H6

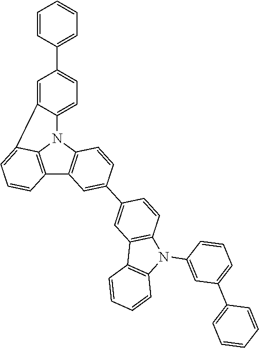 [SMILES: c1ccc(-c2cccc(-n3c4ccccc4c4cc(-c5ccc6c(c5)c5cccc7c8cc(-c9ccccc9)ccc8n6c75)ccc43)c2)cc1]

H7

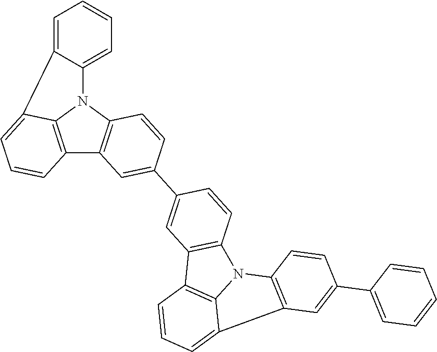 [SMILES: c1ccc(-c2ccc3c(c2)c2cccc4c5cc(-c6ccc7c(c6)c6cccc8c9ccccc9n7c86)ccc5n3c24)cc1]

H8

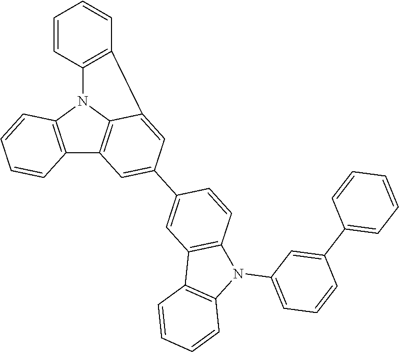 [SMILES: c1ccc(-c2cccc(-n3c4ccccc4c4cc(-c5cc6c7ccccc7n7c8ccccc8c(c5)c67)ccc43)c2)cc1]

H9

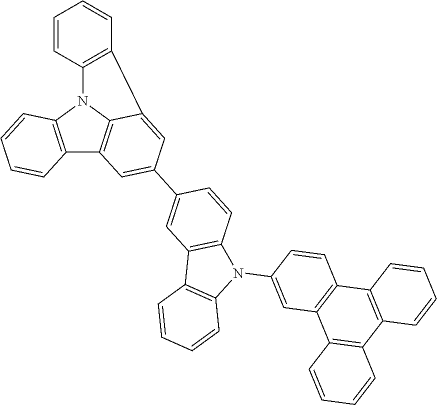 [SMILES: c1ccc2c(c1)c1ccccc1c1cc(-n3c4ccccc4c4cc(-c5cc6c7ccccc7n7c8ccccc8c(c5)c67)ccc43)ccc21]

H10

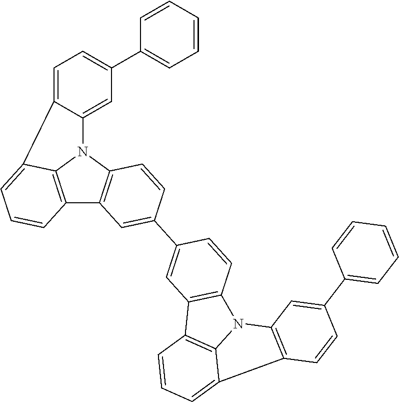 [SMILES: c1ccc(-c2ccc3c4cccc5c6cc(-c7ccc8c(c7)c7cccc9c%10ccc(-c%11ccccc%11)cc%10n8c79)ccc6n(c3c2)c54)cc1]

H11

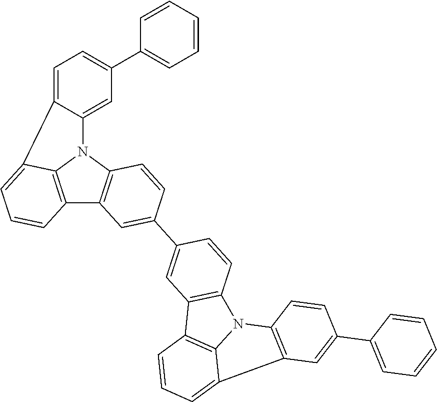 [SMILES: c1ccc(-c2ccc3c(c2)c2cccc4c5cc(-c6ccc7c(c6)c6cccc8c9ccc(-c%10ccccc%10)cc9n7c68)ccc5n3c24)cc1]

H12

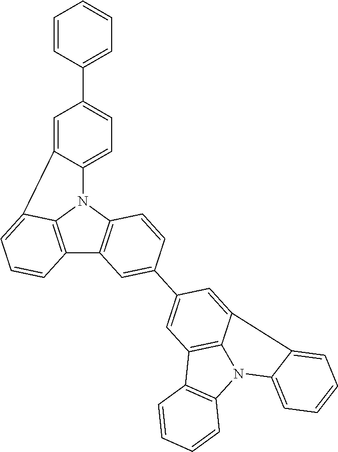 [SMILES: c1ccc(-c2ccc3c(c2)c2cccc4c5cc(-c6cc7c8ccccc8n8c9ccccc9c(c6)c78)ccc5n3c24)cc1]

H13

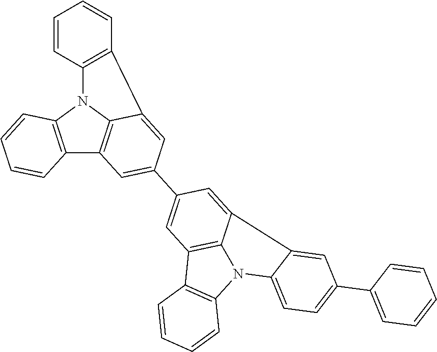 [SMILES: c1ccc(-c2ccc3c(c2)c2cc(-c4cc5c6ccccc6n6c7ccccc7c(c4)c56)cc4c5ccccc5n3c42)cc1]

H14

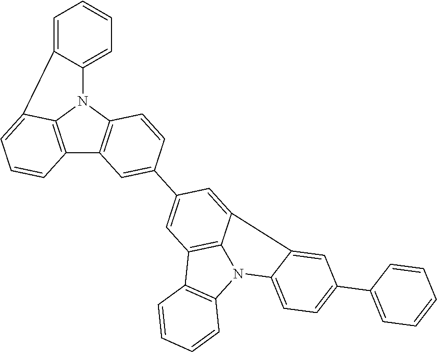 [SMILES: c1ccc(-c2ccc3c(c2)c2cc(-c4ccc5c(c4)c4cccc6c7ccccc7n5c64)cc4c5ccccc5n3c42)cc1]

H15

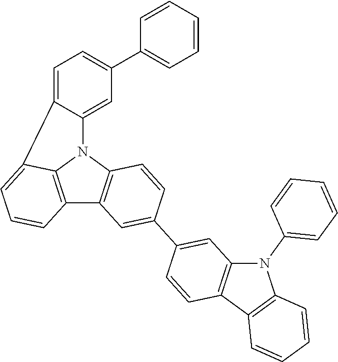 [SMILES: c1ccc(-c2ccc3c4cccc5c6cc(-c7ccc8c9ccccc9n(-c9ccccc9)c8c7)ccc6n(c3c2)c54)cc1]

H16

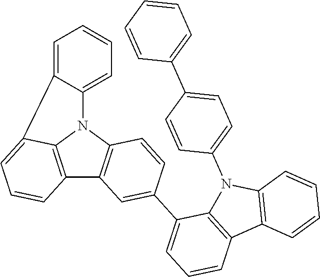 [SMILES: c1ccc(-c2ccc(-n3c4ccccc4c4cccc(-c5ccc6c(c5)c5cccc7c8ccccc8n6c75)c43)cc2)cc1]

H17

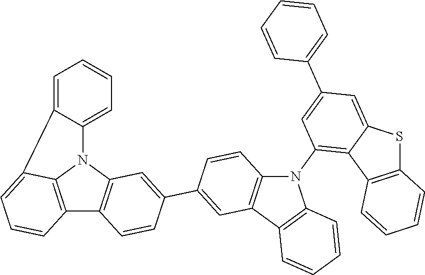 [SMILES: c1ccc(-c2cc(-n3c4ccccc4c4cc(-c5ccc6c7cccc8c9ccccc9n(c6c5)c87)ccc43)c3c(c2)sc2ccccc23)cc1]

H18

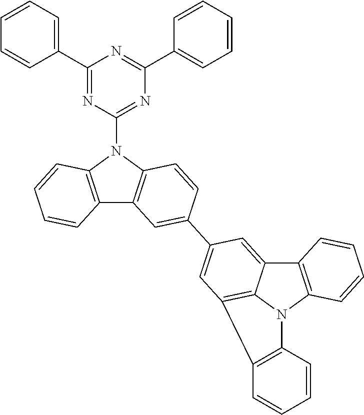 [SMILES: c1ccc(-c2nc(-c3ccccc3)nc(-n3c4ccccc4c4cc(-c5cc6c7ccccc7n7c8ccccc8c(c5)c67)ccc43)n2)cc1]

H19

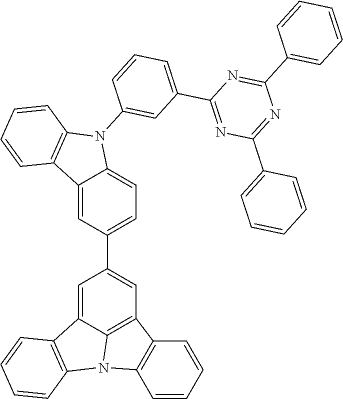 [SMILES: c1ccc(-c2nc(-c3ccccc3)nc(-c3cccc(-n4c5ccccc5c5cc(-c6cc7c8ccccc8n8c9ccccc9c(c6)c78)ccc54)c3)n2)cc1]

H20

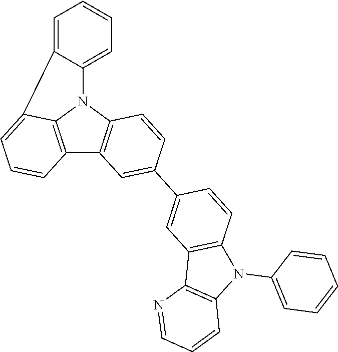 [SMILES: c1ccc(-n2c3ccc(-c4ccc5c(c4)c4cccc6c7ccccc7n5c64)cc3c3ncccc32)cc1]

H21.

Very particularly suitable compounds of the formula (2) that are used in the electroluminescent device of the invention preferably in combination with at least one compound of the formula (1) are the compounds H1, H3, H4, H5, H6, H7, H8, H11 and H12.

The preparation of the compounds of the formula (2) or of the preferred compounds of the formulae (2), (2a), (2b), (2c), (2d) and (2e) and of the compounds from table 3 and compounds H1 to H21 is known to the person skilled in the art. The compounds can be prepared by synthesis steps known to those skilled in the art, for example bromination, Suzuki coupling, Ullmann coupling, Hartwig-Buchwald coupling, etc. A suitable synthesis method is shown in general terms in scheme 2 below, where the symbols and indices used have the definitions given above.

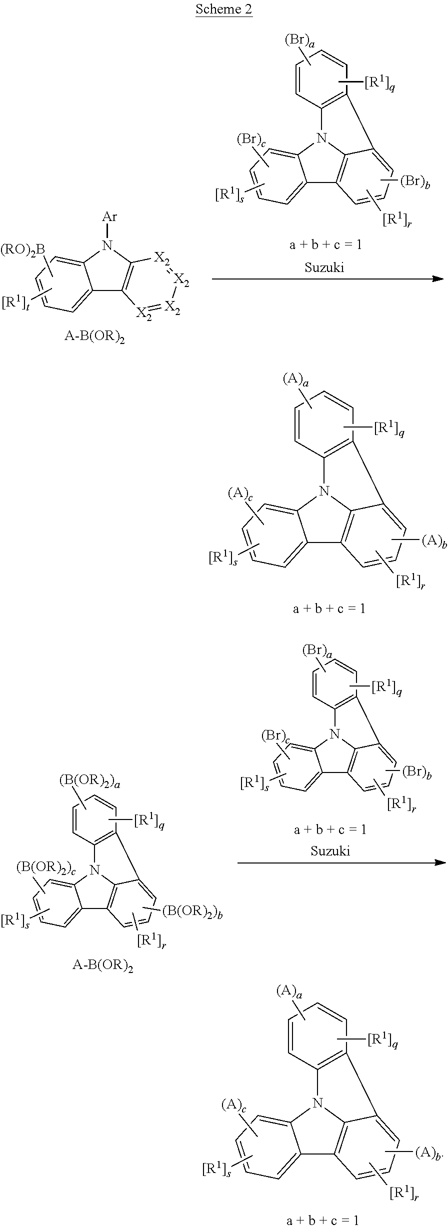

The aforementioned host materials of the formula (1) and the embodiments thereof that are described as preferred or the compounds from table 1 and the compounds E1 to E54 and E60 to E69 can be combined as desired in the device of the invention with the host materials of the formulae (2), (2a), (2b), (2c), (2d) and (2e) mentioned and the embodiments thereof that are described as preferred or the compounds from table 3 or the compounds H1 to H21.

The invention likewise further provides mixtures comprising at least one compound of the formula (1) as host material 1 and at least one compound of the formula (2) as host material 2

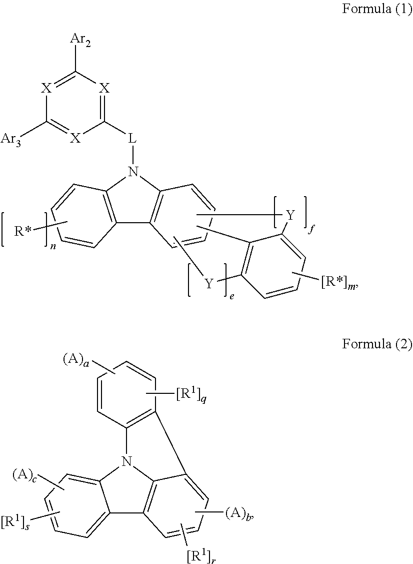

where the symbols and indices used are as follows:
X is the same or different at each instance and is CR⁰ or N, where at least two symbols X are N;
X₂ is the same or different at each instance and is CH, CR¹ or N, where not more than 2 symbols X₂ can be N;
Y is the same or different at each instance and is selected from C(R)₂ and NR;
L is the same or different at each instance and is a single bond or phenylene;
R* at each instance is independently D or an aromatic or heteroaromatic ring system that has 6 to 18 ring atoms and may be partly or fully deuterated:
R # is the same or different at each instance and is selected from the group consisting of D, F, Cl, Br, I, CN, NO₂, C(═O)R², P(═O)(Ar₁)₂, P(Ar₁)₂, B(Ar₁)₂, Si(Ar₁)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR² and where one or more hydrogen atoms may be replaced by D, F, Cl, Br, I, CN or NO₂, an aromatic or heteroaromatic ring system that has 5 to 40 ring atoms and may be substituted in each case by one or more R² radicals, an aryloxy or heteroaryloxy group that has 5 to 40 ring atoms and may be substituted by one or more R² radicals, or an aralkyl or heteroaralkyl group that has 5 to 40 ring atoms and may be substituted by one or more R² radicals;
R is the same or different at each instance and is selected from a straight-chain alkyl group having 1 to 20 carbon atoms or a branched or cyclic alkyl group having 3 to 20 carbon atoms, an aromatic or heteroaromatic ring system having 5 to 40 ring atoms, or an aralkyl or heteroaralkyl group having 5 to 40 ring atoms; at the same time, two substituents R bonded to the same carbon atom or to adjacent carbon atoms may form a monocyclic or polycyclic, aliphatic, aromatic or heteroaromatic ring system that may be substituted by one or more R² radicals;
R¹ is the same or different at each instance and is selected from the group consisting of CN, a straight-chain alkyl, alkoxy or thioalkyl group having 1 to 20 carbon atoms or a branched or cyclic alkyl, alkoxy or thioalkyl group having 3 to 20 carbon atoms, an aromatic or heteroaromatic ring system having 5 to 40 ring atoms, an aryloxy or heteroaryloxy group having 5 to 40 ring atoms, or an aralkyl or heteroaralkyl group having 5 to 40 ring atoms; at the same time, it is possible for two substituents R¹ bonded to the same carbon atom or to adjacent carbon atoms to form a monocyclic or polycyclic, aliphatic, aromatic or heteroaromatic ring system that may be substituted by one or more R² radicals;
R⁰ and R² are the same or different at each instance and are selected from the group consisting of H, D, F, Cl, Br, I, CN, NO₂, N(Ar₁)₂, NH₂, N(R³)₂, C(═O)Ar₁, C(═O)H, C(═O)R³, P(═O)(Ar₁)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R³ radicals, where one or more nonadjacent CH₂ groups may be replaced by HC═CH, R³C═CR³, C≡C, Si(R³)₂, Ge(R³)₂, Sn(R³)₂, C═O, C═S, C═Se, C═NR³, P(═O)(R³), SO, SO₂, NH, NR³, O, S, CONH or CONR³ and where one or more hydrogen atoms may be replaced by D, F, Cl, Br, I, CN or NO₂, an aromatic or heteroaromatic ring system that has 5 to 60 ring atoms and may be substituted in each case by one or more R³ radicals, an aryloxy or heteroaryloxy group that has 5 to 60 ring atoms and may be substituted by one or more R³ radicals, or a combination of these systems, where optionally two or more adjacent substituents R² may form a monocyclic or polycyclic, aliphatic, aromatic or heteroaromatic ring system that may be substituted by one or more R³ radicals;
R³ is the same or different at each instance and is selected from the group consisting of H, D, F, CN, an aliphatic hydrocarbyl radical having 1 to 20 carbon atoms, or an aromatic or heteroaromatic ring system having 5 to 30 ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents together to form a mono- or polycyclic, aliphatic ring system;
Ar₁ is the same or different at each instance and is an aromatic or heteroaromatic ring system which has 5 to 30 ring atoms and may be substituted by one or more nonaromatic R³ radicals; at the same time, two Ar₁ radicals bonded to the same nitrogen atom, phosphorus atom or boron atom may also be bridged to one another by a single bond or a bridge selected from N(R³), C(R³)₂, O or S;
Ar₂ and Ar₃ are different at each instance;
Ar₂ at each instance is a biphenyl, a dibenzofuranyl, a dibenzothiophenyl, a carbazol-N-yl or a carbazol-N-yl-phenyl group that may be substituted by one or more R* radicals;
Ar₃ at each instance is an aryl or heteroaryl group that has 5 to 40 ring atoms and may be substituted by one or more R² radicals;
A at each instance is independently a group of the formula (3) or (4),

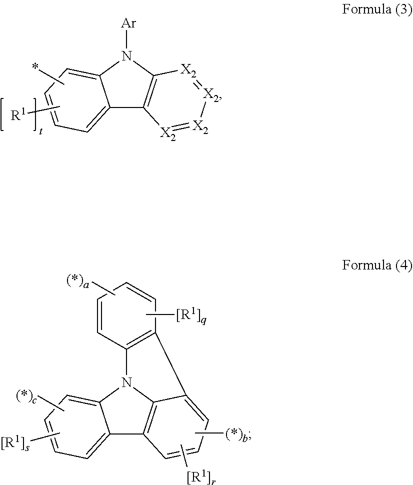

Ar at each instance is in each case independently an aryl group which has 6 to 40 ring atoms and may be substituted by one or more R # radicals, or a heteroaryl group which has 5 to 40 ring atoms and may be substituted by one or more R # radicals;
* indicates the binding site to the formula (2);
a, b, c at each instance are each independently 0 or 1, where the sum total of the indices a+b+c at each instance is 1:
e, f at each instance are each independently 0 or 1, where the sum total of the indices e+f at each instance is 1;
   n and m at each instance are independently 0, 1, 2, 3 or 4; and
q, r, s, t at each instance are each independently 0 or 1.

The details with regard to the host materials of the formulae (1) and (2) and the preferred embodiments thereof are correspondingly also applicable to the mixture of the invention.

Particularly preferred mixtures of the host materials of the formula (1) with the host materials of the formula (2) for the device of the invention are obtained by combination of the compounds E1 to E54 and E60 to E69 with the compounds from table 3.

Very particularly preferred mixtures of the host materials of the formula (1) with the host materials of the formula (2) for the device of the invention are obtained by combination of the compounds E1 to E54 and E60 to E69 with the compounds H1 to H21, as shown in table 5 below.

TABLE 5

| | | | | | | | | |
|---|---|---|---|---|---|---|---|---|
| M1 | E1 | H1 | M2 | E2 | H1 | M3 | E3 | H1 |
| M4 | E4 | H1 | M5 | E5 | H1 | M6 | E6 | H1 |
| M7 | E7 | H1 | M8 | E8 | H1 | M9 | E9 | H1 |
| M10 | E10 | H1 | M11 | E11 | H1 | M12 | E12 | H1 |
| M13 | E13 | H1 | M14 | E14 | H1 | M15 | E15 | H1 |
| M16 | E16 | H1 | M17 | E17 | H1 | M18 | E18 | H1 |
| M19 | E19 | H1 | M20 | E20 | H1 | M21 | E21 | H1 |
| M22 | E22 | H1 | M23 | E23 | H1 | M24 | E24 | H1 |
| M25 | E25 | H1 | M26 | E26 | H1 | M27 | E27 | H1 |
| M28 | E28 | H1 | M29 | E29 | H1 | M30 | E30 | H1 |
| M31 | E31 | H1 | M32 | E32 | H1 | M33 | E33 | H1 |
| M34 | E34 | H1 | M35 | E35 | H1 | M36 | E36 | H1 |
| M37 | E37 | H1 | M38 | E38 | H1 | M39 | E39 | H1 |
| M40 | E40 | H1 | M41 | E41 | H1 | M42 | E42 | H1 |
| M43 | E43 | H1 | M44 | E44 | H1 | M45 | E45 | H1 |
| M46 | E46 | H1 | M47 | E47 | H1 | M48 | E48 | H1 |
| M49 | E49 | H1 | M50 | E50 | H1 | M51 | E51 | H1 |
| M52 | E52 | H1 | M53 | E53 | H1 | M54 | E54 | H1 |
| M55 | E1 | H2 | M56 | E2 | H2 | M57 | E3 | H2 |
| M58 | E4 | H2 | M59 | E5 | H2 | M60 | E6 | H2 |
| M61 | E7 | H2 | M62 | E8 | H2 | M63 | E9 | H2 |
| M64 | E10 | H2 | M65 | E11 | H2 | M66 | E12 | H2 |
| M67 | E13 | H2 | M68 | E14 | H2 | M69 | E15 | H2 |
| M70 | E16 | H2 | M71 | E17 | H2 | M72 | E18 | H2 |
| M73 | E19 | H2 | M74 | E20 | H2 | M75 | E21 | H2 |
| M76 | E22 | H2 | M77 | E23 | H2 | M78 | E24 | H2 |
| M79 | E25 | H2 | M80 | E26 | H2 | M81 | E27 | H2 |
| M82 | E28 | H2 | M83 | E29 | H2 | M84 | E30 | H2 |
| M85 | E31 | H2 | M86 | E32 | H2 | M87 | E33 | H2 |
| M88 | E34 | H2 | M89 | E35 | H2 | M90 | E36 | H2 |
| M91 | E37 | H2 | M92 | E38 | H2 | M93 | E39 | H2 |
| M94 | E40 | H2 | M95 | E41 | H2 | M96 | E42 | H2 |
| M97 | E43 | H2 | M98 | E44 | H2 | M99 | E45 | H2 |
| M100 | E46 | H2 | M101 | E47 | H2 | M102 | E48 | H2 |
| M103 | E49 | H2 | M104 | E50 | H2 | M105 | E51 | H2 |
| M106 | E52 | H2 | M107 | E53 | H2 | M108 | E54 | H2 |
| M109 | E1 | H3 | M110 | E2 | H3 | M111 | E3 | H3 |
| M112 | E4 | H3 | M113 | E5 | H3 | M114 | E6 | H3 |
| M115 | E7 | H3 | M116 | E8 | H3 | M117 | E9 | H3 |
| M118 | E10 | H3 | M119 | E11 | H3 | M120 | E12 | H3 |
| M121 | E13 | H3 | M122 | E14 | H3 | M123 | E15 | H3 |
| M124 | E16 | H3 | M125 | E17 | H3 | M126 | E18 | H3 |
| M127 | E19 | H3 | M128 | E20 | H3 | M129 | E21 | H3 |
| M130 | E22 | H3 | M131 | E23 | H3 | M132 | E24 | H3 |
| M133 | E25 | H3 | M134 | E26 | H3 | M135 | E27 | H3 |
| M136 | E28 | H3 | M137 | E29 | H3 | M138 | E30 | H3 |
| M139 | E31 | H3 | M140 | E32 | H3 | M141 | E33 | H3 |
| M142 | E34 | H3 | M143 | E35 | H3 | M144 | E36 | H3 |
| M145 | E37 | H3 | M146 | E38 | H3 | M147 | E39 | H3 |
| M148 | E40 | H3 | M149 | E41 | H3 | M150 | E42 | H3 |
| M151 | E43 | H3 | M152 | E44 | H3 | M153 | E45 | H3 |
| M154 | E46 | H3 | M155 | E47 | H3 | M156 | E48 | H3 |
| M157 | E49 | H3 | M158 | E50 | H3 | M159 | E51 | H3 |
| M160 | E52 | H3 | M161 | E53 | H3 | M162 | E54 | H3 |
| M163 | E1 | H4 | M164 | E2 | H4 | M165 | E3 | H4 |
| M166 | E4 | H4 | M167 | E5 | H4 | M168 | E6 | H4 |
| M169 | E7 | H4 | M170 | E8 | H4 | M171 | E9 | H4 |
| M172 | E10 | H4 | M173 | E11 | H4 | M174 | E12 | H4 |
| M175 | E13 | H4 | M176 | E14 | H4 | M177 | E15 | H4 |
| M178 | E16 | H4 | M179 | E17 | H4 | M180 | E18 | H4 |
| M181 | E19 | H4 | M182 | E20 | H4 | M183 | E21 | H4 |
| M184 | E22 | H4 | M185 | E23 | H4 | M186 | E24 | H4 |
| M187 | E25 | H4 | M188 | E26 | H4 | M189 | E27 | H4 |
| M190 | E28 | H4 | M191 | E29 | H4 | M192 | E30 | H4 |
| M193 | E31 | H4 | M194 | E32 | H4 | M195 | E33 | H4 |
| M196 | E34 | H4 | M197 | E35 | H4 | M198 | E36 | H4 |
| M199 | E37 | H4 | M200 | E38 | H4 | M201 | E39 | H4 |
| M202 | E40 | H4 | M203 | E41 | H4 | M204 | E42 | H4 |
| M205 | E43 | H4 | M206 | E44 | H4 | M207 | E45 | H4 |
| M208 | E46 | H4 | M209 | E47 | H4 | M210 | E48 | H4 |
| M211 | E49 | H4 | M212 | E50 | H4 | M213 | E51 | H4 |
| M214 | E52 | H4 | M215 | E53 | H4 | M216 | E54 | H4 |
| M217 | E1 | H5 | M218 | E2 | H5 | M219 | E3 | H5 |
| M220 | E4 | H5 | M221 | E5 | H5 | M222 | E6 | H5 |
| M223 | E7 | H5 | M224 | E8 | H5 | M225 | E9 | H5 |
| M226 | E10 | H5 | M227 | E11 | H5 | M228 | E12 | H5 |
| M229 | E13 | H5 | M230 | E14 | H5 | M231 | E15 | H5 |
| M232 | E16 | H5 | M233 | E17 | H5 | M234 | E18 | H5 |
| M235 | E19 | H5 | M236 | E20 | H5 | M237 | E21 | H5 |
| M238 | E22 | H5 | M239 | E23 | H5 | M240 | E24 | H5 |
| M241 | E25 | H5 | M242 | E26 | H5 | M243 | E27 | H5 |
| M244 | E28 | H5 | M245 | E29 | H5 | M246 | E30 | H5 |
| M247 | E31 | H5 | M248 | E32 | H5 | M249 | E33 | H5 |
| M250 | E34 | H5 | M251 | E35 | H5 | M252 | E36 | H5 |
| M253 | E37 | H5 | M254 | E38 | H5 | M255 | E39 | H5 |
| M256 | E40 | H5 | M257 | E41 | H5 | M258 | E42 | H5 |
| M259 | E43 | H5 | M260 | E44 | H5 | M261 | E45 | H5 |
| M262 | E46 | H5 | M263 | E47 | H5 | M264 | E48 | H5 |
| M265 | E49 | H5 | M266 | E50 | H5 | M267 | E51 | H5 |
| M268 | E52 | H5 | M269 | E53 | H5 | M270 | E54 | H5 |
| M271 | E1 | H6 | M272 | E2 | H6 | M273 | E3 | H6 |
| M274 | E4 | H6 | M275 | E5 | H6 | M276 | E6 | H6 |
| M277 | E7 | H6 | M278 | E8 | H6 | M279 | E9 | H6 |
| M280 | E10 | H6 | M281 | E11 | H6 | M282 | E12 | H6 |
| M283 | E13 | H6 | M284 | E14 | H6 | M285 | E15 | H6 |
| M286 | E16 | H6 | M287 | E17 | H6 | M288 | E18 | H6 |
| M289 | E19 | H6 | M290 | E20 | H6 | M291 | E21 | H6 |
| M292 | E22 | H6 | M293 | E23 | H6 | M294 | E24 | H6 |
| M295 | E25 | H6 | M296 | E26 | H6 | M297 | E27 | H6 |
| M298 | E28 | H6 | M299 | E29 | H6 | M300 | E30 | H6 |
| M301 | E31 | H6 | M302 | E32 | H6 | M303 | E33 | H6 |
| M304 | E34 | H6 | M305 | E35 | H6 | M306 | E36 | H6 |
| M307 | E37 | H6 | M308 | E38 | H6 | M309 | E39 | H6 |
| M310 | E40 | H6 | M311 | E41 | H6 | M312 | E42 | H6 |
| M313 | E43 | H6 | M314 | E44 | H6 | M315 | E45 | H6 |
| M316 | E46 | H6 | M317 | E47 | H6 | M318 | E48 | H6 |
| M319 | E49 | H6 | M320 | E50 | H6 | M321 | E51 | H6 |
| M322 | E52 | H6 | M323 | E53 | H6 | M324 | E54 | H6 |
| M325 | E1 | H7 | M326 | E2 | H7 | M327 | E3 | H7 |
| M328 | E4 | H7 | M329 | E5 | H7 | M330 | E6 | H7 |
| M331 | E7 | H7 | M332 | E8 | H7 | M333 | E9 | H7 |
| M334 | E10 | H7 | M335 | E11 | H7 | M336 | E12 | H7 |
| M337 | E13 | H7 | M338 | E14 | H7 | M339 | E15 | H7 |
| M340 | E16 | H7 | M341 | E17 | H7 | M342 | E18 | H7 |
| M343 | E19 | H7 | M344 | E20 | H7 | M345 | E21 | H7 |
| M346 | E22 | H7 | M347 | E23 | H7 | M348 | E24 | H7 |
| M349 | E25 | H7 | M350 | E26 | H7 | M351 | E27 | H7 |
| M352 | E28 | H7 | M353 | E29 | H7 | M354 | E30 | H7 |
| M355 | E31 | H7 | M356 | E32 | H7 | M357 | E33 | H7 |
| M358 | E34 | H7 | M359 | E35 | H7 | M360 | E36 | H7 |
| M361 | E37 | H7 | M362 | E38 | H7 | M363 | E39 | H7 |
| M364 | E40 | H7 | M365 | E41 | H7 | M366 | E42 | H7 |
| M367 | E43 | H7 | M368 | E44 | H7 | M369 | E45 | H7 |
| M370 | E46 | H7 | M371 | E47 | H7 | M372 | E48 | H7 |
| M373 | E49 | H7 | M374 | E50 | H7 | M375 | E51 | H7 |
| M376 | E52 | H7 | M377 | E53 | H7 | M378 | E54 | H7 |
| M379 | E1 | H8 | M380 | E2 | H8 | M381 | E3 | H8 |
| M382 | E4 | H8 | M383 | E5 | H8 | M384 | E6 | H8 |
| M385 | E7 | H8 | M386 | E8 | H8 | M387 | E9 | H8 |
| M388 | E10 | H8 | M389 | E11 | H8 | M390 | E12 | H8 |
| M391 | E13 | H8 | M392 | E14 | H8 | M393 | E15 | H8 |
| M394 | E16 | H8 | M395 | E17 | H8 | M396 | E18 | H8 |
| M397 | E19 | H8 | M398 | E20 | H8 | M399 | E21 | H8 |
| M400 | E22 | H8 | M401 | E23 | H8 | M402 | E24 | H8 |
| M403 | E25 | H8 | M404 | E26 | H8 | M405 | E27 | H8 |
| M406 | E28 | H8 | M407 | E29 | H8 | M408 | E30 | H8 |
| M409 | E31 | H8 | M410 | E32 | H8 | M411 | E33 | H8 |
| M412 | E34 | H8 | M413 | E35 | H8 | M414 | E36 | H8 |
| M415 | E37 | H8 | M416 | E38 | H8 | M417 | E39 | H8 |
| M418 | E40 | H8 | M419 | E41 | H8 | M420 | E42 | H8 |
| M421 | E43 | H8 | M422 | E44 | H8 | M423 | E45 | H8 |
| M424 | E46 | H8 | M425 | E47 | H8 | M426 | E48 | H8 |
| M427 | E49 | H8 | M428 | E50 | H8 | M429 | E51 | H8 |
| M430 | E52 | H8 | M431 | E53 | H8 | M432 | E54 | H8 |
| M433 | E1 | H9 | M434 | E2 | H9 | M435 | E3 | H9 |
| M436 | E4 | H9 | M437 | E5 | H9 | M438 | E6 | H9 |
| M439 | E7 | H9 | M440 | E8 | H9 | M441 | E9 | H9 |
| M442 | E10 | H9 | M443 | E11 | H9 | M444 | E12 | H9 |
| M445 | E13 | H9 | M446 | E14 | H9 | M447 | E15 | H9 |
| M448 | E16 | H9 | M449 | E17 | H9 | M450 | E18 | H9 |
| M451 | E19 | H9 | M452 | E20 | H9 | M453 | E21 | H9 |
| M454 | E22 | H9 | M455 | E23 | H9 | M456 | E24 | H9 |
| M457 | E25 | H9 | M458 | E26 | H9 | M459 | E27 | H9 |
| M460 | E28 | H9 | M461 | E29 | H9 | M462 | E30 | H9 |
| M463 | E31 | H9 | M464 | E32 | H9 | M465 | E33 | H9 |
| M466 | E34 | H9 | M467 | E35 | H9 | M468 | E36 | H9 |
| M469 | E37 | H9 | M470 | E38 | H9 | M471 | E39 | H9 |
| M472 | E40 | H9 | M473 | E41 | H9 | M474 | E42 | H9 |
| M475 | E43 | H9 | M476 | E44 | H9 | M477 | E45 | H9 |
| M478 | E46 | H9 | M479 | E47 | H9 | M480 | E48 | H9 |
| M481 | E49 | H9 | M482 | E50 | H9 | M483 | E51 | H9 |
| M484 | E52 | H9 | M485 | E53 | H9 | M486 | E54 | H9 |
| M487 | E1 | H10 | M488 | E2 | H10 | M489 | E3 | H10 |
| M490 | E4 | H10 | M491 | E5 | H10 | M492 | E6 | H10 |
| M493 | E7 | H10 | M494 | E8 | H10 | M495 | E9 | H10 |
| M496 | E10 | H10 | M497 | E11 | H10 | M498 | E12 | H10 |
| M499 | E13 | H10 | M500 | E14 | H10 | M501 | E15 | H10 |
| M502 | E16 | H10 | M503 | E17 | H10 | M504 | E18 | H10 |
| M505 | E19 | H10 | M506 | E20 | H10 | M507 | E21 | H10 |
| M508 | E22 | H10 | M509 | E23 | H10 | M510 | E24 | H10 |
| M511 | E25 | H10 | M512 | E26 | H10 | M513 | E27 | H10 |
| M514 | E28 | H10 | M515 | E29 | H10 | M516 | E30 | H10 |
| M517 | E31 | H10 | M518 | E32 | H10 | M519 | E33 | H10 |
| M520 | E34 | H10 | M521 | E35 | H10 | M522 | E36 | H10 |
| M523 | E37 | H10 | M524 | E38 | H10 | M525 | E39 | H10 |
| M526 | E40 | H10 | M527 | E41 | H10 | M528 | E42 | H10 |
| M529 | E43 | H10 | M530 | E44 | H10 | M531 | E45 | H10 |
| M532 | E46 | H10 | M533 | E47 | H10 | M534 | E48 | H10 |
| M535 | E49 | H10 | M536 | E50 | H10 | M537 | E51 | H10 |
| M538 | E52 | H10 | M539 | E53 | H10 | M540 | E54 | H10 |
| M541 | E1 | H11 | M542 | E2 | H11 | M543 | E3 | H11 |
| M544 | E4 | H11 | M545 | E5 | H11 | M546 | E6 | H11 |
| M547 | E7 | H11 | M548 | E8 | H11 | M549 | E9 | H11 |
| M550 | E10 | H11 | M551 | E11 | H11 | M552 | E12 | H11 |
| M553 | E13 | H11 | M554 | E14 | H11 | M555 | E15 | H11 |
| M556 | E16 | H11 | M557 | E17 | H11 | M558 | E18 | H11 |
| M559 | E19 | H11 | M560 | E20 | H11 | M561 | E21 | H11 |
| M562 | E22 | H11 | M563 | E23 | H11 | M564 | E24 | H11 |
| M565 | E25 | H11 | M566 | E26 | H11 | M567 | E27 | H11 |
| M568 | E28 | H11 | M569 | E29 | H11 | M570 | E30 | H11 |
| M571 | E31 | H11 | M572 | E32 | H11 | M573 | E33 | H11 |
| M574 | E34 | H11 | M575 | E35 | H11 | M576 | E36 | H11 |
| M577 | E37 | H11 | M578 | E38 | H11 | M579 | E39 | H11 |
| M580 | E40 | H11 | M581 | E41 | H11 | M582 | E42 | H11 |
| M583 | E43 | H11 | M584 | E44 | H11 | M585 | E45 | H11 |
| M586 | E46 | H11 | M587 | E47 | H11 | M588 | E48 | H11 |
| M589 | E49 | H11 | M590 | E50 | H11 | M591 | E51 | H11 |
| M592 | E52 | H11 | M593 | E53 | H11 | M594 | E54 | H11 |
| M595 | E1 | H12 | M596 | E2 | H12 | M597 | E3 | H12 |
| M598 | E4 | H12 | M599 | E5 | H12 | M600 | E6 | H12 |
| M601 | E7 | H12 | M602 | E8 | H12 | M603 | E9 | H12 |
| M604 | E10 | H12 | M605 | E11 | H12 | M606 | E12 | H12 |
| M607 | E13 | H12 | M608 | E14 | H12 | M609 | E15 | H12 |
| M610 | E16 | H12 | M611 | E17 | H12 | M612 | E18 | H12 |
| M613 | E19 | H12 | M614 | E20 | H12 | M615 | E21 | H12 |
| M616 | E22 | H12 | M617 | E23 | H12 | M618 | E24 | H12 |
| M619 | E25 | H12 | M620 | E26 | H12 | M621 | E27 | H12 |
| M622 | E28 | H12 | M623 | E29 | H12 | M624 | E30 | H12 |
| M625 | E31 | H12 | M626 | E32 | H12 | M627 | E33 | H12 |
| M628 | E34 | H12 | M629 | E35 | H12 | M630 | E36 | H12 |
| M631 | E37 | H12 | M632 | E38 | H12 | M633 | E39 | H12 |
| M634 | E40 | H12 | M635 | E41 | H12 | M636 | E42 | H12 |
| M637 | E43 | H12 | M638 | E44 | H12 | M639 | E45 | H12 |
| M640 | E46 | H12 | M641 | E47 | H12 | M642 | E48 | H12 |
| M643 | E49 | H12 | M644 | E50 | H12 | M645 | E51 | H12 |
| M646 | E52 | H12 | M647 | E53 | H12 | M648 | E54 | H12 |
| M649 | E1 | H13 | M650 | E2 | H13 | M651 | E3 | H13 |
| M652 | E4 | H13 | M653 | E5 | H13 | M654 | E6 | H13 |
| M655 | E7 | H13 | M656 | E8 | H13 | M657 | E9 | H13 |
| M658 | E10 | H13 | M659 | E11 | H13 | M660 | E12 | H13 |
| M661 | E13 | H13 | M662 | E14 | H13 | M663 | E15 | H13 |
| M664 | E16 | H13 | M665 | E17 | H13 | M666 | E18 | H13 |
| M667 | E19 | H13 | M668 | E20 | H13 | M669 | E21 | H13 |
| M670 | E22 | H13 | M671 | E23 | H13 | M672 | E24 | H13 |
| M673 | E25 | H13 | M674 | E26 | H13 | M675 | E27 | H13 |
| M676 | E28 | H13 | M677 | E29 | H13 | M678 | E30 | H13 |
| M679 | E31 | H13 | M680 | E32 | H13 | M681 | E33 | H13 |
| M682 | E34 | H13 | M683 | E35 | H13 | M684 | E36 | H13 |
| M685 | E37 | H13 | M686 | E38 | H13 | M687 | E39 | H13 |
| M688 | E40 | H13 | M689 | E41 | H13 | M690 | E42 | H13 |
| M691 | E43 | H13 | M692 | E44 | H13 | M693 | E45 | H13 |
| M694 | E46 | H13 | M695 | E47 | H13 | M696 | E48 | H13 |
| M697 | E49 | H13 | M698 | E50 | H13 | M699 | E51 | H13 |
| M700 | E52 | H13 | M701 | E53 | H13 | M702 | E54 | H13 |
| M703 | E1 | H14 | M704 | E2 | H14 | M705 | E3 | H14 |
| M706 | E4 | H14 | M707 | E5 | H14 | M708 | E6 | H14 |
| M709 | E7 | H14 | M710 | E8 | H14 | M711 | E9 | H14 |
| M712 | E10 | H14 | M713 | E11 | H14 | M714 | E12 | H14 |
| M715 | E13 | H14 | M716 | E14 | H14 | M717 | E15 | H14 |
| M718 | E16 | H14 | M719 | E17 | H14 | M720 | E18 | H14 |
| M721 | E19 | H14 | M722 | E20 | H14 | M723 | E21 | H14 |
| M724 | E22 | H14 | M725 | E23 | H14 | M726 | E24 | H14 |
| M727 | E25 | H14 | M728 | E26 | H14 | M729 | E27 | H14 |
| M730 | E28 | H14 | M731 | E29 | H14 | M732 | E30 | H14 |
| M733 | E31 | H14 | M734 | E32 | H14 | M735 | E33 | H14 |
| M736 | E34 | H14 | M737 | E35 | H14 | M738 | E36 | H14 |
| M739 | E37 | H14 | M740 | E38 | H14 | M741 | E39 | H14 |
| M742 | E40 | H14 | M743 | E41 | H14 | M744 | E42 | H14 |
| M745 | E43 | H14 | M746 | E44 | H14 | M747 | E45 | H14 |
| M748 | E46 | H14 | M749 | E47 | H14 | M750 | E48 | H14 |
| M751 | E49 | H14 | M752 | E50 | H14 | M753 | E51 | H14 |
| M754 | E52 | H14 | M755 | E53 | H14 | M756 | E54 | H14 |
| M757 | E1 | H15 | M758 | E2 | H15 | M759 | E3 | H15 |
| M760 | E4 | H15 | M761 | E5 | H15 | M762 | E6 | H15 |
| M763 | E7 | H15 | M764 | E8 | H15 | M765 | E9 | H15 |
| M766 | E10 | H15 | M767 | E11 | H15 | M768 | E12 | H15 |
| M769 | E13 | H15 | M770 | E14 | H15 | M771 | E15 | H15 |
| M772 | E16 | H15 | M773 | E17 | H15 | M774 | E18 | H15 |
| M775 | E19 | H15 | M776 | E20 | H15 | M777 | E21 | H15 |
| M778 | E22 | H15 | M779 | E23 | H15 | M780 | E24 | H15 |
| M781 | E25 | H15 | M782 | E26 | H15 | M783 | E27 | H15 |
| M784 | E28 | H15 | M785 | E29 | H15 | M786 | E30 | H15 |
| M787 | E31 | H15 | M788 | E32 | H15 | M789 | E33 | H15 |
| M790 | E34 | H15 | M791 | E35 | H15 | M792 | E36 | H15 |
| M793 | E37 | H15 | M794 | E38 | H15 | M795 | E39 | H15 |
| M796 | E40 | H15 | M797 | E41 | H15 | M798 | E42 | H15 |
| M799 | E43 | H15 | M800 | E44 | H15 | M801 | E45 | H15 |
| M802 | E46 | H15 | M803 | E47 | H15 | M804 | E48 | H15 |
| M805 | E49 | H15 | M806 | E50 | H15 | M807 | E51 | H15 |
| M808 | E52 | H15 | M809 | E53 | H15 | M810 | E54 | H15 |
| M811 | E1 | H16 | M812 | E2 | H16 | M813 | E3 | H16 |
| M814 | E4 | H16 | M815 | E5 | H16 | M816 | E6 | H16 |
| M817 | E7 | H16 | M818 | E8 | H16 | M819 | E9 | H16 |
| M820 | E10 | H16 | M821 | E11 | H16 | M822 | E12 | H16 |
| M823 | E13 | H16 | M824 | E14 | H16 | M825 | E15 | H16 |
| M826 | E16 | H16 | M827 | E17 | H16 | M828 | E18 | H16 |
| M829 | E19 | H16 | M830 | E20 | H16 | M831 | E21 | H16 |
| M832 | E22 | H16 | M833 | E23 | H16 | M834 | E24 | H16 |
| M835 | E25 | H16 | M836 | E26 | H16 | M837 | E27 | H16 |
| M838 | E28 | H16 | M839 | E29 | H16 | M840 | E30 | H16 |
| M841 | E31 | H16 | M842 | E32 | H16 | M843 | E33 | H16 |
| M844 | E34 | H16 | M845 | E35 | H16 | M846 | E36 | H16 |
| M847 | E37 | H16 | M848 | E38 | H16 | M849 | E39 | H16 |
| M850 | E40 | H16 | M851 | E41 | H16 | M852 | E42 | H16 |
| M853 | E43 | H16 | M854 | E44 | H16 | M855 | E45 | H16 |
| M856 | E46 | H16 | M857 | E47 | H16 | M858 | E48 | H16 |
| M859 | E49 | H16 | M860 | E50 | H16 | M861 | E51 | H16 |
| M862 | E52 | H16 | M863 | E53 | H16 | M864 | E54 | H16 |
| M865 | E1 | H17 | M866 | E2 | H17 | M867 | E3 | H17 |
| M868 | E4 | H17 | M869 | E5 | H17 | M870 | E6 | H17 |
| M871 | E7 | H17 | M872 | E8 | H17 | M873 | E9 | H17 |
| M874 | E10 | H17 | M875 | E11 | H17 | M876 | E12 | H17 |
| M877 | E13 | H17 | M878 | E14 | H17 | M879 | E15 | H17 |
| M880 | E16 | H17 | M881 | E17 | H17 | M882 | E18 | H17 |
| M883 | E19 | H17 | M884 | E20 | H17 | M885 | E21 | H17 |
| M886 | E22 | H17 | M887 | E23 | H17 | M888 | E24 | H17 |
| M889 | E25 | H17 | M890 | E26 | H17 | M891 | E27 | H17 |
| M892 | E28 | H17 | M893 | E29 | H17 | M894 | E30 | H17 |
| M895 | E31 | H17 | M896 | E32 | H17 | M897 | E33 | H17 |
| M898 | E34 | H17 | M899 | E35 | H17 | M900 | E36 | H17 |
| M901 | E37 | H17 | M902 | E38 | H17 | M903 | E39 | H17 |
| M904 | E40 | H17 | M905 | E41 | H17 | M906 | E42 | H17 |
| M907 | E43 | H17 | M908 | E44 | H17 | M909 | E45 | H17 |
| M910 | E46 | H17 | M911 | E47 | H17 | M912 | E48 | H17 |
| M913 | E49 | H17 | M914 | E50 | H17 | M915 | E51 | H17 |
| M916 | E52 | H17 | M917 | E53 | H17 | M918 | E54 | H17 |
| M919 | E1 | H18 | M920 | E2 | H18 | M921 | E3 | H18 |
| M922 | E4 | H18 | M923 | E5 | H18 | M924 | E6 | H18 |
| M925 | E7 | H18 | M926 | E8 | H18 | M927 | E9 | H18 |
| M928 | E10 | H18 | M929 | E11 | H18 | M930 | E12 | H18 |
| M931 | E13 | H18 | M932 | E14 | H18 | M933 | E15 | H18 |
| M934 | E16 | H18 | M935 | E17 | H18 | M936 | E18 | H18 |
| M937 | E19 | H18 | M938 | E20 | H18 | M939 | E21 | H18 |
| M940 | E22 | H18 | M941 | E23 | H18 | M942 | E24 | H18 |
| M943 | E25 | H18 | M944 | E26 | H18 | M945 | E27 | H18 |
| M946 | E28 | H18 | M947 | E29 | H18 | M948 | E30 | H18 |
| M949 | E31 | H18 | M950 | E32 | H18 | M951 | E33 | H18 |
| M952 | E34 | H18 | M953 | E35 | H18 | M954 | E36 | H18 |
| M955 | E37 | H18 | M956 | E38 | H18 | M957 | E39 | H18 |
| M958 | E40 | H18 | M959 | E41 | H18 | M960 | E42 | H18 |
| M961 | E43 | H18 | M962 | E44 | H18 | M963 | E45 | H18 |
| M964 | E46 | H18 | M965 | E47 | H18 | M966 | E48 | H18 |
| M967 | E49 | H18 | M968 | E50 | H18 | M969 | E51 | H18 |
| M970 | E52 | H18 | M971 | E53 | H18 | M972 | E54 | H18 |
| M973 | E1 | H19 | M974 | E2 | H19 | M975 | E3 | H19 |
| M976 | E4 | H19 | M977 | E5 | H19 | M978 | E6 | H19 |
| M979 | E7 | H19 | M980 | E8 | H19 | M981 | E9 | H19 |
| M982 | E10 | H19 | M983 | E11 | H19 | M984 | E12 | H19 |
| M985 | E13 | H19 | M986 | E14 | H19 | M987 | E15 | H19 |
| M988 | E16 | H19 | M989 | E17 | H19 | M990 | E18 | H19 |
| M991 | E19 | H19 | M992 | E20 | H19 | M993 | E21 | H19 |
| M994 | E22 | H19 | M995 | E23 | H19 | M996 | E24 | H19 |
| M997 | E25 | H19 | M998 | E26 | H19 | M999 | E27 | H19 |
| M1000 | E28 | H19 | M1001 | E29 | H19 | M1002 | E30 | H19 |
| M1003 | E31 | H19 | M1004 | E32 | H19 | M1005 | E33 | H19 |
| M1006 | E34 | H19 | M1007 | E35 | H19 | M1008 | E36 | H19 |
| M1009 | E37 | H19 | M1010 | E38 | H19 | M1011 | E39 | H19 |
| M1012 | E40 | H19 | M1013 | E41 | H19 | M1014 | E42 | H19 |
| M1015 | E43 | H19 | M1016 | E44 | H19 | M1017 | E45 | H19 |
| M1018 | E46 | H19 | M1019 | E47 | H19 | M1020 | E48 | H19 |
| M1021 | E49 | H19 | M1022 | E50 | H19 | M1023 | E51 | H19 |
| M1024 | E52 | H19 | M1025 | E53 | H19 | M1026 | E54 | H19 |
| M1027 | E1 | H20 | M1028 | E2 | H20 | M1029 | E3 | H20 |
| M1030 | E4 | H20 | M1031 | E5 | H20 | M1032 | E6 | H20 |
| M1033 | E7 | H20 | M1034 | E8 | H20 | M1035 | E9 | H20 |
| M1036 | E10 | H20 | M1037 | E11 | H20 | M1038 | E12 | H20 |
| M1039 | E13 | H20 | M1040 | E14 | H20 | M1041 | E15 | H20 |
| M1042 | E16 | H20 | M1043 | E17 | H20 | M1044 | E18 | H20 |
| M1045 | E19 | H20 | M1046 | E20 | H20 | M1047 | E21 | H20 |
| M1048 | E22 | H20 | M1049 | E23 | H20 | M1050 | E24 | H20 |
| M1051 | E25 | H20 | M1052 | E26 | H20 | M1053 | E27 | H20 |
| M1054 | E28 | H20 | M1055 | E29 | H20 | M1056 | E30 | H20 |
| M1057 | E31 | H20 | M1058 | E32 | H20 | M1059 | E33 | H20 |
| M1060 | E34 | H20 | M1061 | E35 | H20 | M1062 | E36 | H20 |
| M1063 | E37 | H20 | M1064 | E38 | H20 | M1065 | E39 | H20 |
| M1066 | E40 | H20 | M1067 | E41 | H20 | M1068 | E42 | H20 |
| M1069 | E43 | H20 | M1070 | E44 | H20 | M1071 | E45 | H20 |
| M1072 | E46 | H20 | M1073 | E47 | H20 | M1074 | E48 | H20 |
| M1075 | E49 | H20 | M1076 | E50 | H20 | M1077 | E51 | H20 |
| M1078 | E52 | H20 | M1079 | E53 | H20 | M1080 | E54 | H20 |
| M1081 | E1 | H21 | M1082 | E2 | H21 | M1083 | E3 | H21 |
| M1084 | E4 | H21 | M1085 | E5 | H21 | M1086 | E6 | H21 |
| M1087 | E7 | H21 | M1088 | E8 | H21 | M1089 | E9 | H21 |
| M1090 | E10 | H21 | M1091 | E11 | H21 | M1092 | E12 | H21 |
| M1093 | E13 | H21 | M1094 | E14 | H21 | M1095 | E15 | H21 |
| M1096 | E16 | H21 | M1097 | E17 | H21 | M1098 | E18 | H21 |
| M1099 | E19 | H21 | M1100 | E20 | H21 | M1101 | E21 | H21 |
| M1102 | E22 | H21 | M1103 | E23 | H21 | M1104 | E24 | H21 |
| M1105 | E25 | H21 | M1106 | E26 | H21 | M1107 | E27 | H21 |
| M1108 | E28 | H21 | M1109 | E29 | H21 | M1110 | E30 | H21 |
| M1111 | E31 | H21 | M1112 | E32 | H21 | M1113 | E33 | H21 |
| M1114 | E34 | H21 | M1115 | E35 | H21 | M1116 | E36 | H21 |
| M1117 | E37 | H21 | M1118 | E38 | H21 | M1119 | E39 | H21 |
| M1120 | E40 | H21 | M1121 | E41 | H21 | M1122 | E42 | H21 |
| M1123 | E43 | H21 | M1124 | E44 | H21 | M1125 | E45 | H21 |
| M1126 | E46 | H21 | M1127 | E47 | H21 | M1128 | E48 | H21 |
| M1129 | E49 | H21 | M1130 | E50 | H21 | M1131 | E51 | H21 |
| M1132 | E52 | H21 | M1133 | E53 | H21 | M1134 | E54 | H21 |
| M1135 | E61 | H1 | M1136 | E61 | H2 | M1137 | E61 | H3 |
| M1138 | E61 | H4 | M1139 | E61 | H5 | M1140 | E61 | H6 |
| M1141 | E61 | H7 | M1142 | E61 | H8 | M1143 | E61 | H9 |
| M1144 | E61 | H10 | M1145 | E61 | H11 | M1146 | E61 | H12 |
| M1147 | E61 | H13 | M1148 | E61 | H14 | M1149 | E61 | H15 |
| M1150 | E61 | H16 | M1151 | E61 | H17 | M1152 | E61 | H18 |
| M1153 | E61 | H19 | M1154 | E61 | H20 | M1155 | E61 | H21 |
| M1156 | E62 | H1 | M1157 | E62 | H2 | M1158 | E62 | H3 |
| M1159 | E62 | H4 | M1160 | E62 | H5 | M1161 | E62 | H6 |
| M1162 | E62 | H7 | M1163 | E62 | H8 | M1164 | E62 | H9 |
| M1165 | E62 | H10 | M1166 | E62 | H11 | M1167 | E62 | H12 |
| M1168 | E62 | H13 | M1169 | E62 | H14 | M1170 | E62 | H15 |
| M1171 | E62 | H16 | M1172 | E62 | H17 | M1173 | E62 | H18 |
| M1174 | E62 | H19 | M1175 | E62 | H20 | M1176 | E62 | H21 |
| M1177 | E63 | H1 | M1178 | E63 | H2 | M1179 | E63 | H3 |
| M1180 | E63 | H4 | M1181 | E63 | H5 | M1182 | E63 | H6 |
| M1183 | E63 | H7 | M1184 | E63 | H8 | M1185 | E63 | H9 |
| M1186 | E63 | H10 | M1187 | E63 | H11 | M1188 | E63 | H12 |
| M1189 | E63 | H13 | M1190 | E63 | H14 | M1191 | E63 | H15 |
| M1192 | E63 | H16 | M1193 | E63 | H17 | M1194 | E63 | H18 |
| M1195 | E63 | H19 | M1196 | E63 | H20 | M1197 | E63 | H21 |
| M1198 | E64 | H1 | M1199 | E64 | H2 | M1200 | E64 | H3 |
| M1201 | E64 | H4 | M1202 | E64 | H5 | M1203 | E64 | H6 |
| M1204 | E64 | H7 | M1205 | E64 | H8 | M1206 | E64 | H9 |
| M1207 | E64 | H10 | M1208 | E64 | H11 | M1209 | E64 | H12 |
| M1210 | E64 | H13 | M1211 | E64 | H14 | M1212 | E64 | H15 |
| M1213 | E64 | H16 | M1214 | E64 | H17 | M1215 | E64 | H18 |
| M1216 | E64 | H19 | M1217 | E64 | H20 | M1218 | E64 | H21 |
| M1219 | E65 | H1 | M1220 | E65 | H2 | M1221 | E65 | H3 |
| M1222 | E65 | H4 | M1223 | E65 | H5 | M1224 | E65 | H6 |
| M1225 | E65 | H7 | M1226 | E65 | H8 | M1227 | E65 | H9 |
| M1228 | E65 | H10 | M1229 | E65 | H11 | M1230 | E65 | H12 |
| M1231 | E65 | H13 | M1232 | E65 | H14 | M1233 | E65 | H15 |
| M1234 | E65 | H16 | M1235 | E65 | H17 | M1236 | E65 | H18 |
| M1237 | E65 | H19 | M1238 | E65 | H20 | M1239 | E65 | H21 |
| M1240 | E66 | H1 | M1241 | E66 | H2 | M1242 | E66 | H3 |
| M1243 | E66 | H4 | M1244 | E66 | H5 | M1245 | E66 | H6 |
| M1246 | E66 | H7 | M1247 | E66 | H8 | M1248 | E66 | H9 |
| M1249 | E66 | H10 | M1250 | E66 | H11 | M1251 | E66 | H12 |
| M1252 | E66 | H13 | M1253 | E66 | H14 | M1254 | E66 | H15 |
| M1255 | E66 | H16 | M1256 | E66 | H17 | M1257 | E66 | H18 |
| M1258 | E66 | H19 | M1259 | E66 | H20 | M1260 | E66 | H21 |
| M1261 | E67 | H1 | M1262 | E67 | H2 | M1263 | E67 | H3 |
| M1264 | E67 | H4 | M1265 | E67 | H5 | M1266 | E67 | H6 |
| M1267 | E67 | H7 | M1268 | E67 | H8 | M1269 | E67 | H9 |
| M1270 | E67 | H10 | M1271 | E67 | H11 | M1272 | E67 | H12 |
| M1273 | E67 | H13 | M1274 | E67 | H14 | M1275 | E67 | H15 |
| M1276 | E67 | H16 | M1277 | E67 | H17 | M1278 | E67 | H18 |
| M1279 | E67 | H19 | M1280 | E67 | H20 | M1281 | E67 | H21 |
| M1282 | E68 | H1 | M1283 | E68 | H2 | M1284 | E68 | H3 |
| M1285 | E68 | H4 | M1286 | E68 | H5 | M1287 | E68 | H6 |
| M1288 | E68 | H7 | M1289 | E68 | H8 | M1290 | E68 | H9 |
| M1291 | E68 | H10 | M1292 | E68 | H11 | M1293 | E68 | H12 |
| M1294 | E68 | H13 | M1295 | E68 | H14 | M1296 | E68 | H15 |
| M1297 | E68 | H16 | M1298 | E68 | H17 | M1299 | E68 | H18 |
| M1300 | E68 | H19 | M1301 | E68 | H20 | M1302 | E68 | H21 |
| M1303 | E69 | H1 | M1304 | E69 | H2 | M1305 | E69 | H3 |
| M1306 | E69 | H4 | M1307 | E69 | H5 | M1308 | E69 | H6 |
| M1309 | E69 | H7 | M1310 | E69 | H8 | M1311 | E69 | H9 |
| M1312 | E69 | H10 | M1313 | E69 | H11 | M1314 | E69 | H12 |
| M1315 | E69 | H13 | M1316 | E69 | H14 | M1317 | E69 | H15 |
| M1318 | E69 | H16 | M1319 | E69 | H17 | M1320 | E69 | H18 |
| M1321 | E69 | H19 | M1322 | E69 | H20 | M1323 | E69 | H21 |
| M1324 | E60 | H1 | M1325 | E60 | H2 | M1326 | E60 | H3 |
| M1327 | E60 | H4 | M1328 | E60 | H5 | M1329 | E60 | H6 |
| M1330 | E60 | H7 | M1331 | E60 | H8 | M1332 | E60 | H9 |
| M1333 | E60 | H10 | M1334 | E60 | H11 | M1335 | E60 | H12 |
| M1336 | E60 | H13 | M1337 | E60 | H14 | M1338 | E60 | H15 |
| M1339 | E60 | H16 | M1340 | E60 | H17 | M1341 | E60 | H18 |
| M1342 | E60 | H19 | M1343 | E60 | H20 | M1344 | E60 | H21. |

The concentration of the electron-transporting host material of the formula (1) as described above or described as preferred in the mixture of the invention or in the light-emitting layer of the device of the invention is in the range from 5% by weight to 90% by weight, preferably in the range from 10% by weight to 85% by weight, more preferably in the range from 20% by weight to 85% by weight, even more preferably in the range from 30% by weight to 80% by weight, very especially preferably in the range from 20% by weight to 60% by weight and most preferably in the range from 30% by weight to 50% by weight, based on the overall mixture or based on the overall composition of the light-emitting layer.

The concentration of the hole-transporting host material of the formula (2) as described above or described as preferred in the mixture of the invention or in the light-emitting layer of the device of the invention is in the range from 10% by weight to 95% by weight, preferably in the range from 15% by weight to 90% by weight, more preferably in the range from 15% by weight to 80% by weight, even more preferably in the range from 20% by weight to 70% by weight, very especially preferably in the range from 40% by weight to 80% by weight and most preferably in the range from 50% by weight to 70% by weight, based on the overall mixture or based on the overall composition of the light-emitting layer.

The present invention also relates to a mixture which, as well as the aforementioned host materials 1 and 2, as described above or described with preference, especially mixtures M1 to M1344, also contains at least one phosphorescent emitter.

The present invention also relates to an organic electroluminescent device as described above or described with preference, wherein the light-emitting layer, as well as the aforementioned host materials 1 and 2, as described above or described with preference, especially material combinations M1 to M1344, also comprises at least one phosphorescent emitter.

The concentration of the phosphorescent emitter as described hereinafter or described as preferred in the mixture of the invention or in the light-emitting layer of the device of the invention is in the range from 1% by weight to 30% by weight, preferably in the range from 2% by weight to 20% by weight, more preferably in the range from 4% by weight to 15% by weight, even more preferably in the range from 8% by weight to 12% by weight, based on the overall mixture or based on the overall composition of the light-emitting layer.

The term “phosphorescent emitters” typically encompasses compounds where the light is emitted through a spin-forbidden transition from an excited state having higher spin multiplicity, i.e. a spin state>1, for example through a transition from a triplet state or a state having an even higher spin quantum number, for example a quintet state. This is preferably understood to mean a transition from a triplet state.

Suitable phosphorescent emitters (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent emitters.

In general, all phosphorescent complexes as used for phosphorescent OLEDs according to the prior art and as known to those skilled in the art in the field of organic electroluminescent devices are suitable.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186, WO 2018/001990, WO 2018/019687, WO 2018/019688, WO 2018/041769, WO 2018/054798, WO 2018/069196, WO 2018/069197, WO 2018/069273, WO 2018/178001, WO 2018/177981, WO 2019/020538, WO 2019/115423, WO 2019/158453 and WO 2019/179909.

Preferred phosphorescent emitters according to the present invention conform to the formula (IIIa)

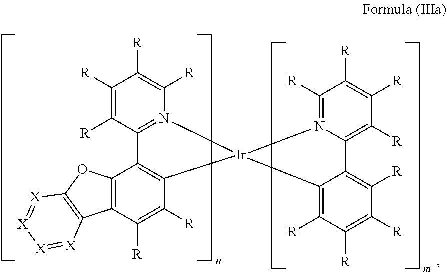

where the symbols and indices for this formula (IIIa) are defined as follows:

n+m is 3, n is 1 or 2, m is 2 or 1,

X is N or CR,

R is H, D, or a branched or linear alkyl group having 1 to 10 carbon atoms or a partly or fully deuterated branched or linear alkyl group having 1 to 10 carbon atoms or a cycloalkyl group which has 4 to 7 carbon atoms and may be partly or fully substituted by deuterium.

The invention accordingly further provides an organic electroluminescent device as described above or described as preferred, characterized in that the light-emitting layer, as well as the host materials 1 and 2, comprises at least one phosphorescent emitter conforming to the formula (IIIa) as described above.

In emitters of the formula (IIIa), n is preferably 1 and m is preferably 2.

In emitters of the formula (IIIa), preferably one X is selected from N and the other X are CR.

In emitters of the formula (IIIa), at least one R is preferably different from H.

In emitters of the formula (IIIa), preferably two R are different from H and have one of the other definitions given above for the emitters of the formula (IIIa).

Preferred phosphorescent emitters according to the present invention conform to the formulae (I), (II) and (III)

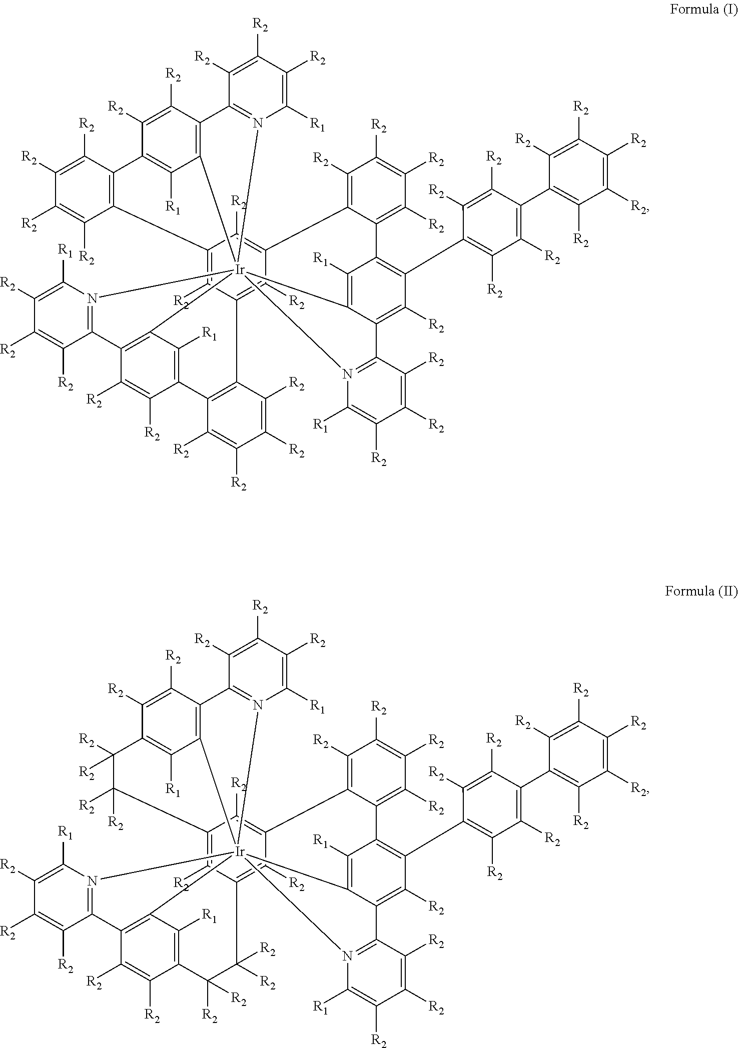

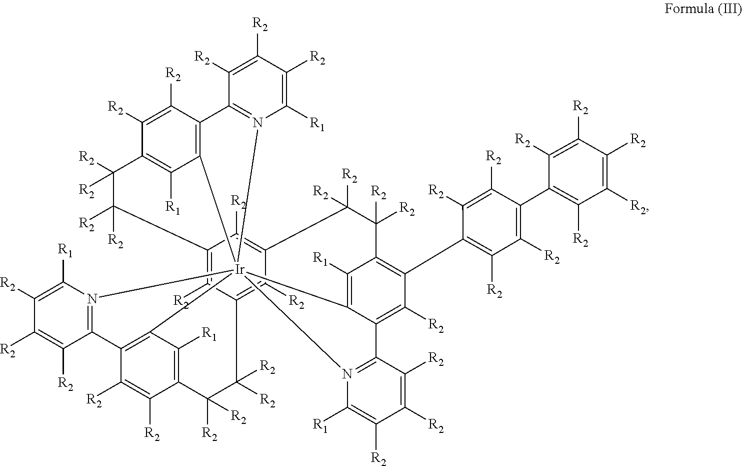

where the symbols and indices for these formulae (1), (II) and (III) are defined as follows:

R¹ is H or D, R² is H, D, or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred phosphorescent emitters according to the present invention conform to the formulae (IV), (V) and (VI)

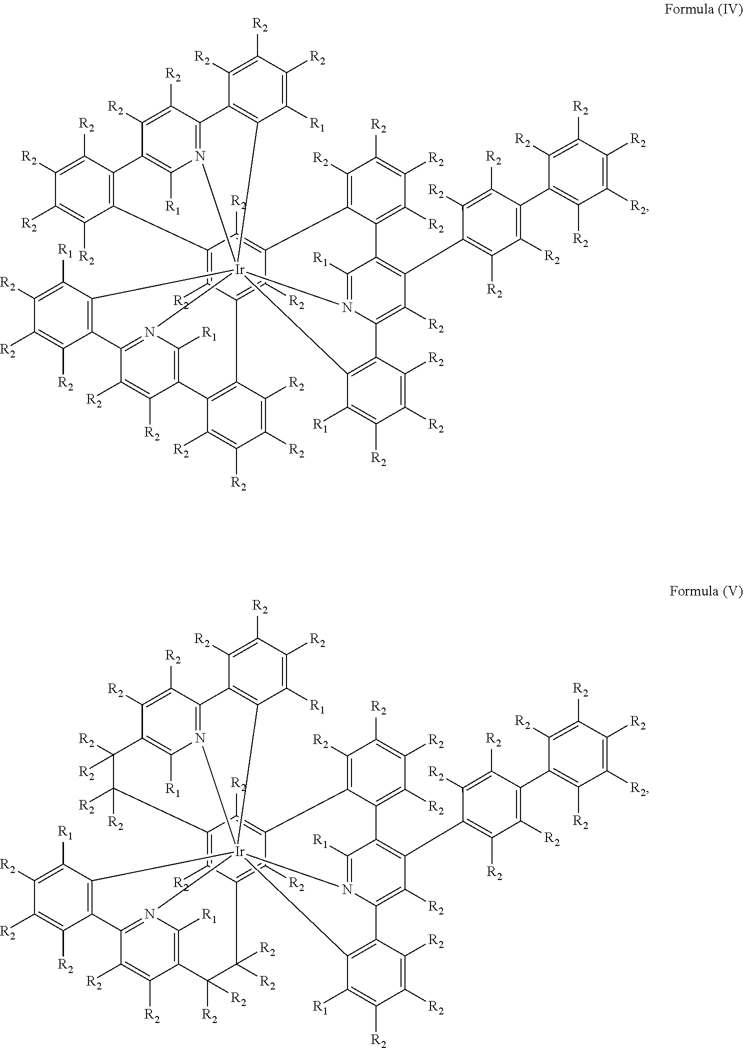

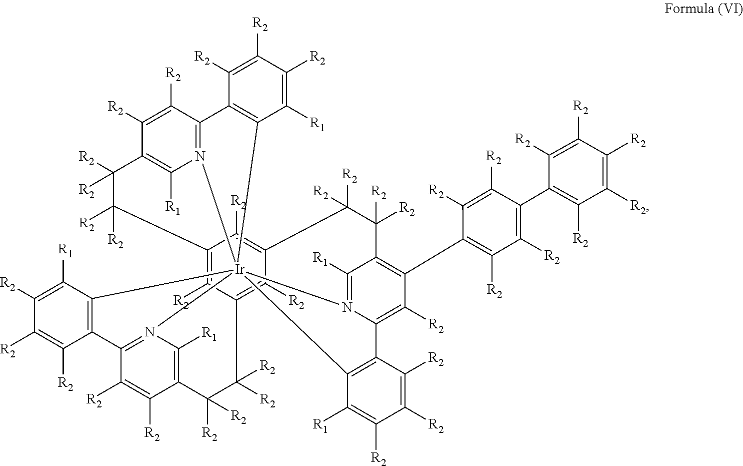

where the symbols and indices for these formulae (IV), (V) and (VI) are defined as follows:

R₁ is H or D, R₂ is H, D, F or a branched or linear alkyl group having 1 to 10 carbon atoms or a partly or fully deuterated branched or linear alkyl group having 1 to 10 carbon atoms or a cycloalkyl group which has 4 to 10 carbon atoms and may be partly or fully substituted by deuterium.

Preferred examples of phosphorescent emitters are listed in table 6 below.

TABLE 6

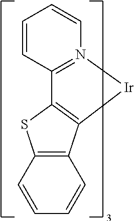

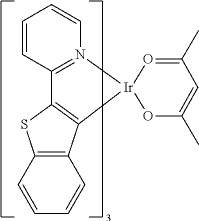

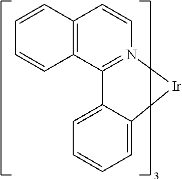

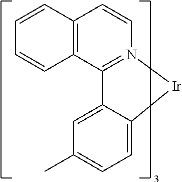

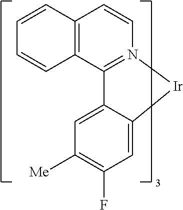

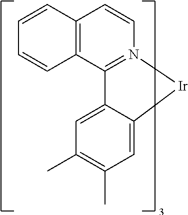

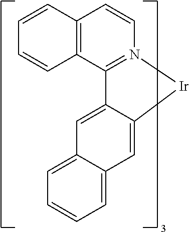

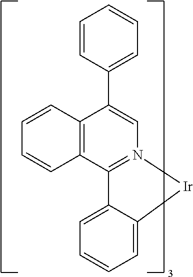

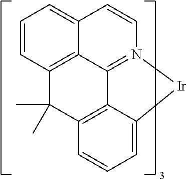

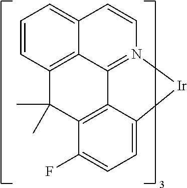

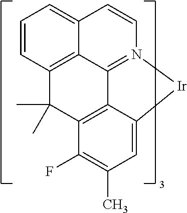

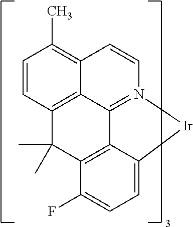

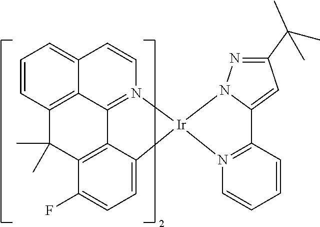

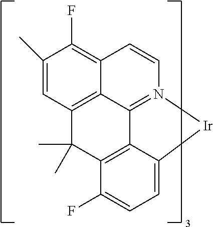

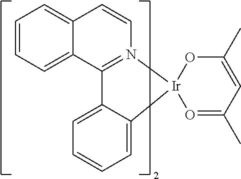

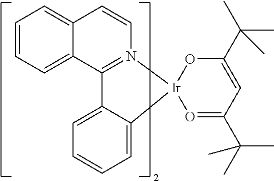

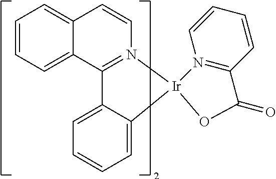

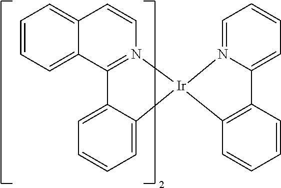

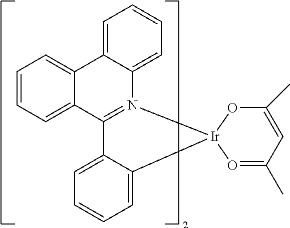

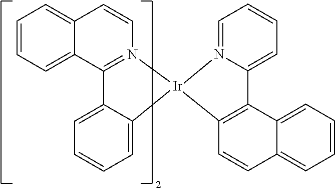

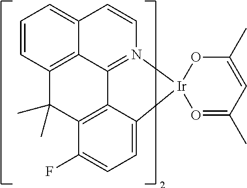

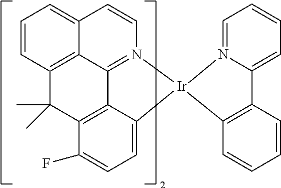

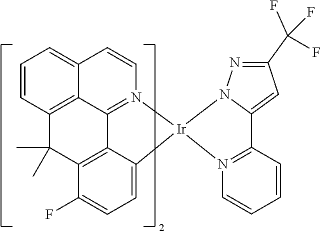

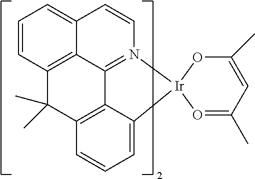

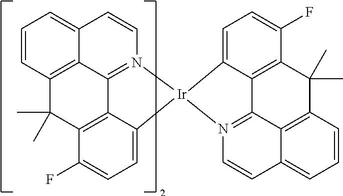

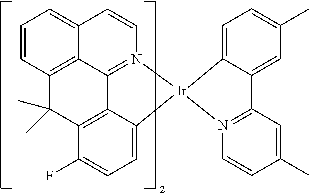

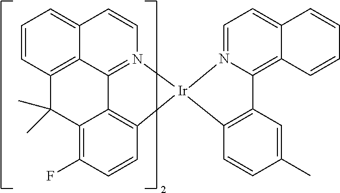

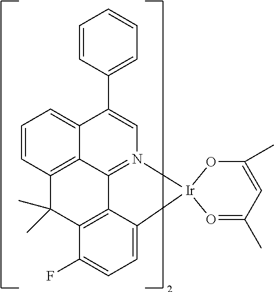

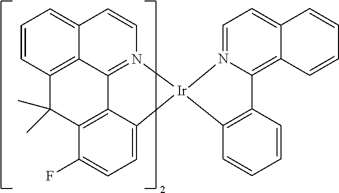

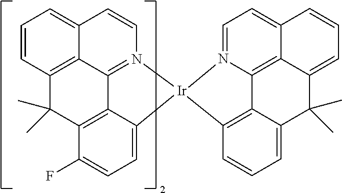

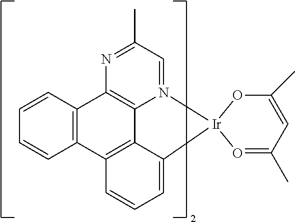

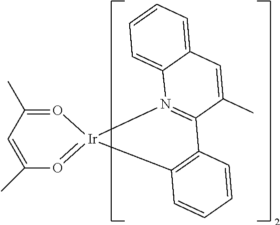

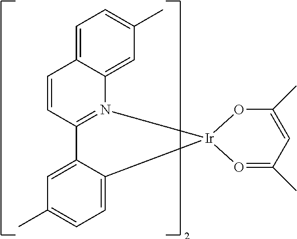

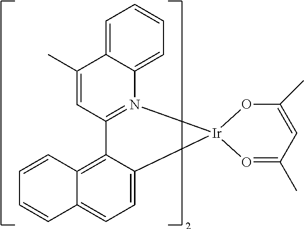

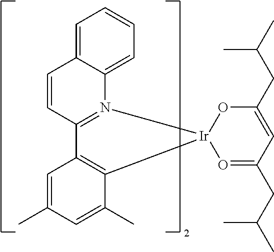

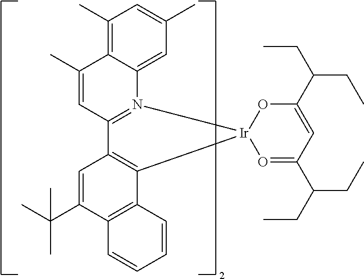

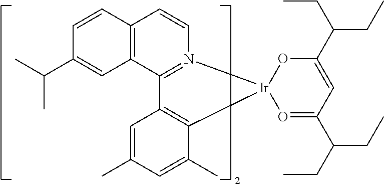

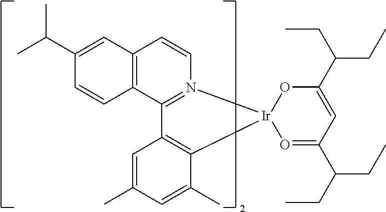

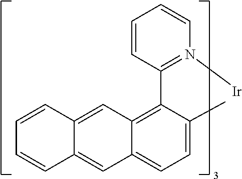

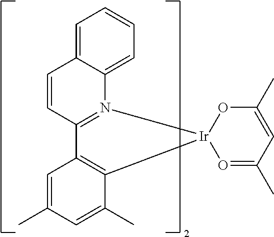

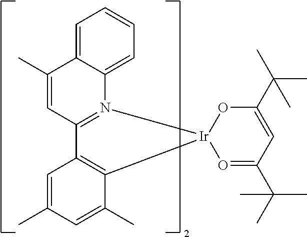

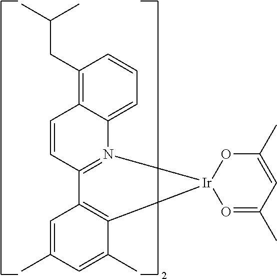

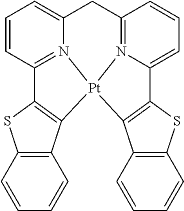

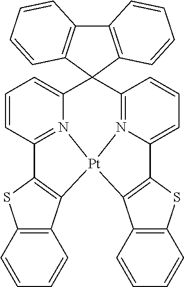

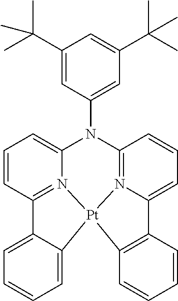

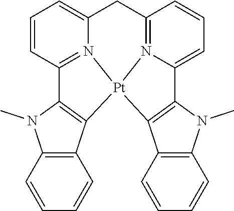

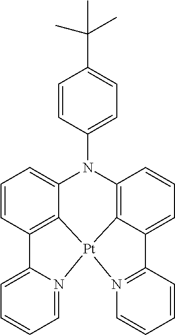

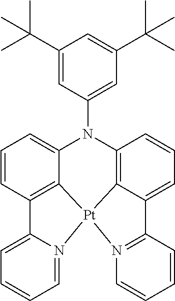

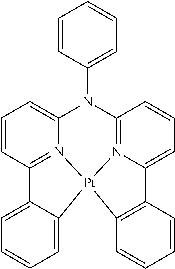

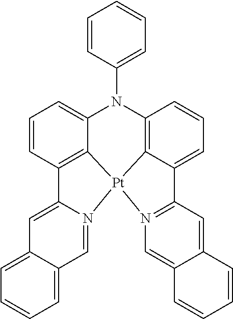

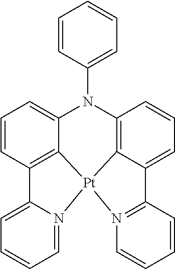

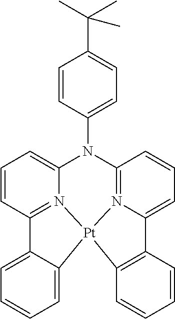

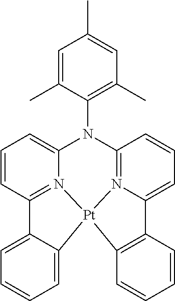

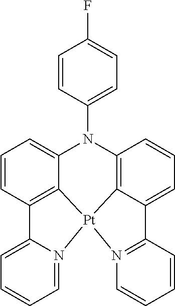

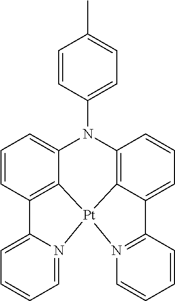

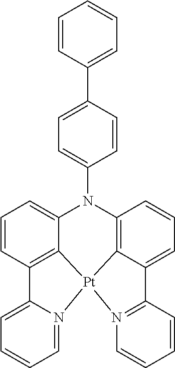

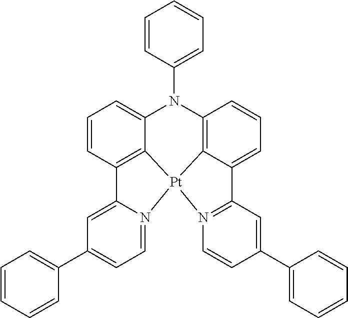

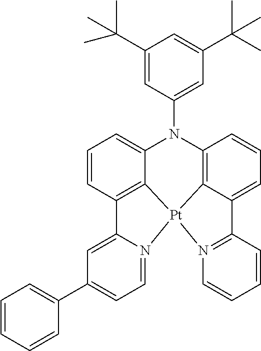

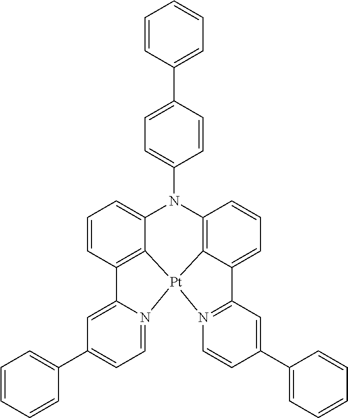

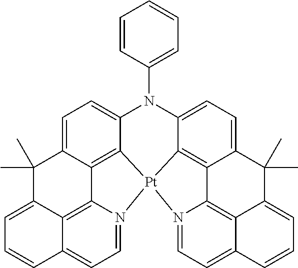

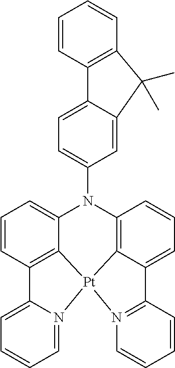

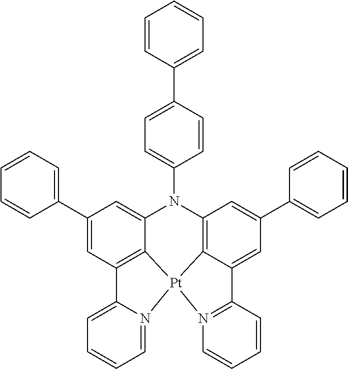

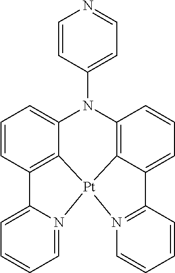

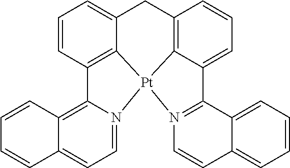

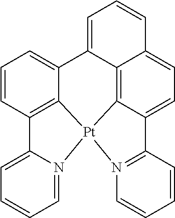

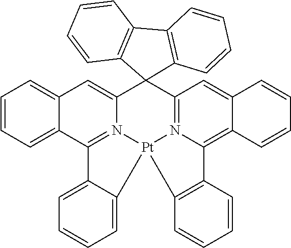

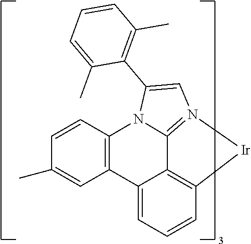

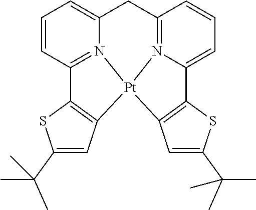

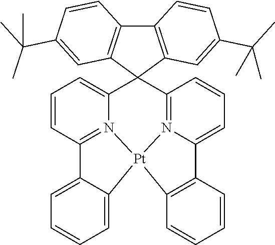

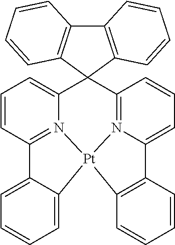

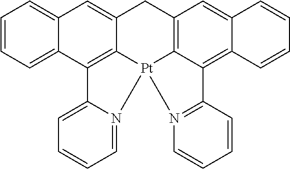

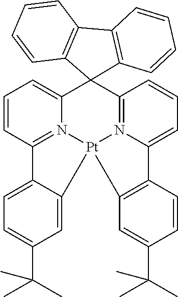

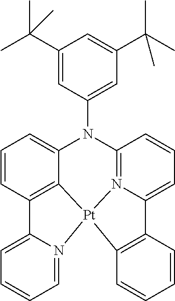

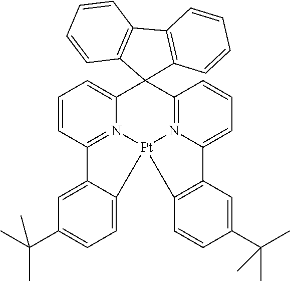

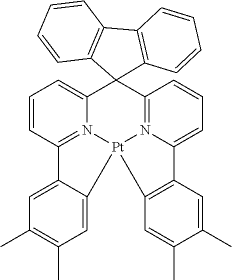

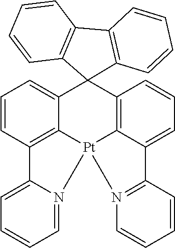

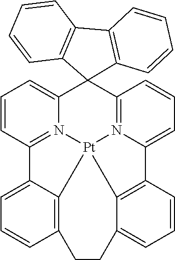

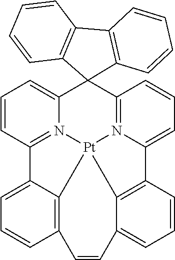

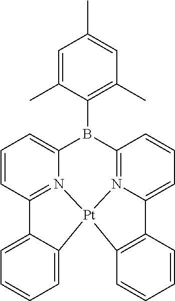

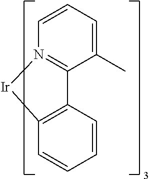

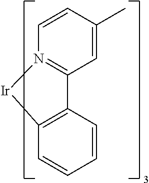

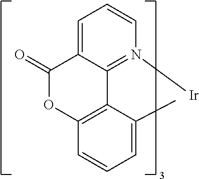

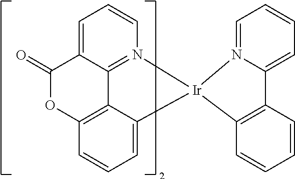

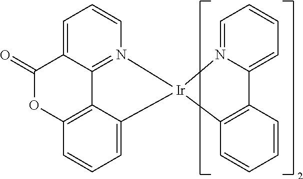

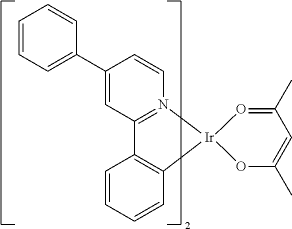

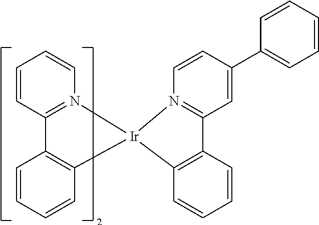

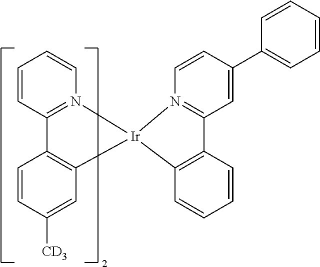

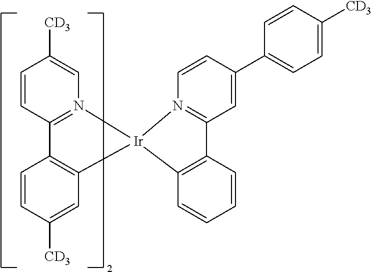

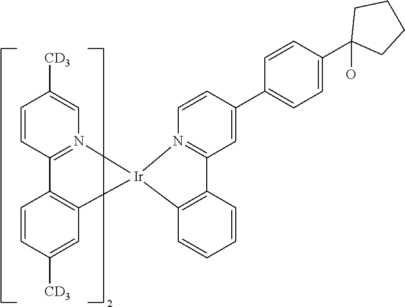

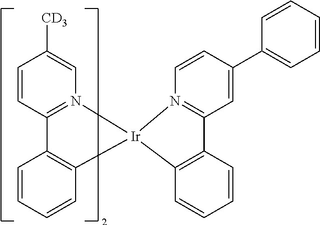

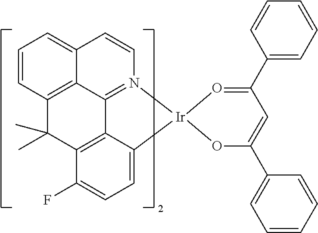

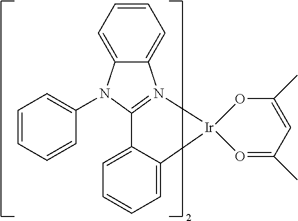

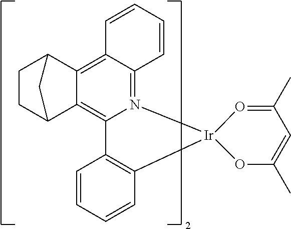

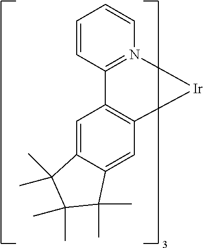

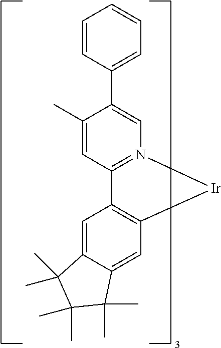

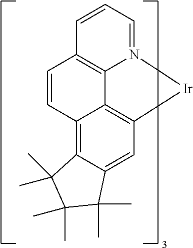

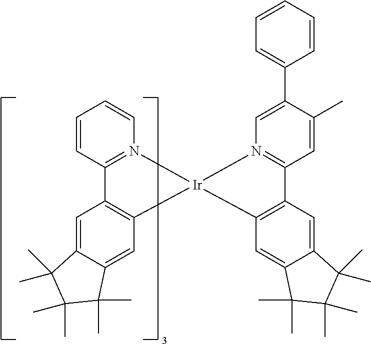

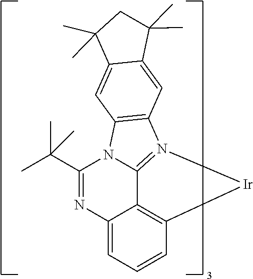

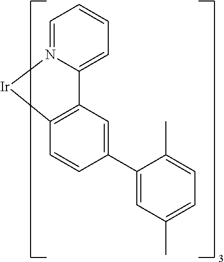

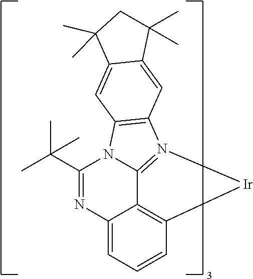

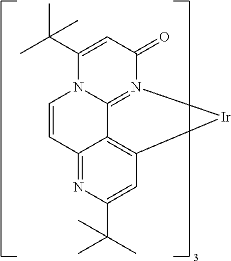

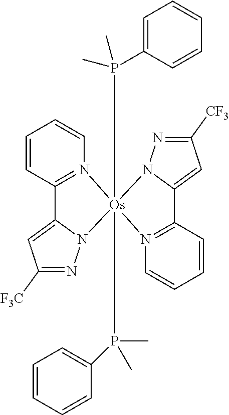

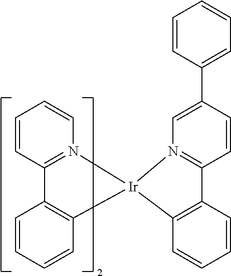

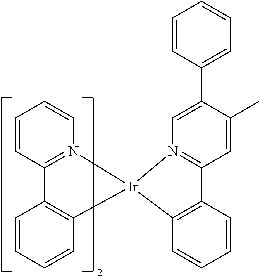

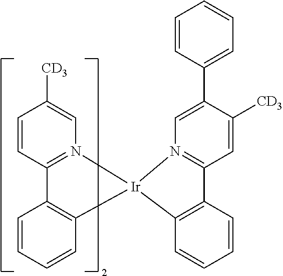

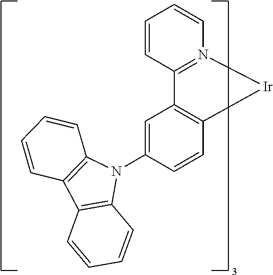

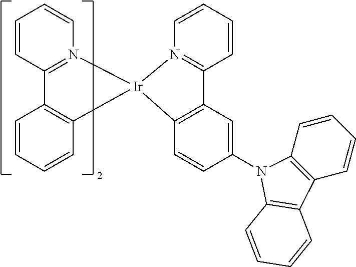

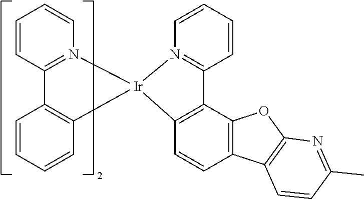

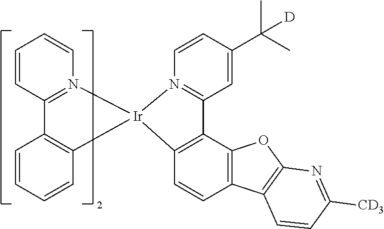

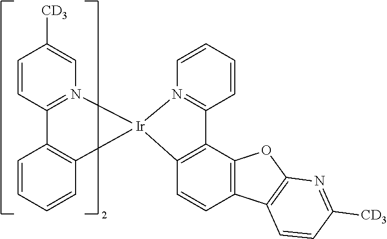

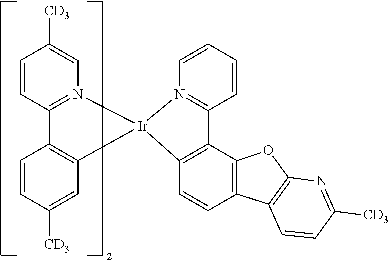

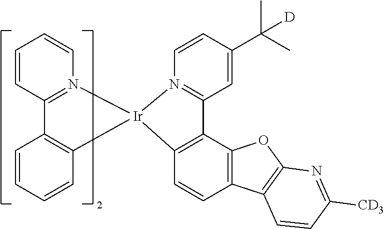

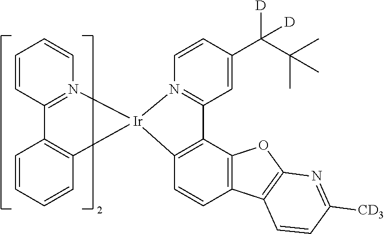

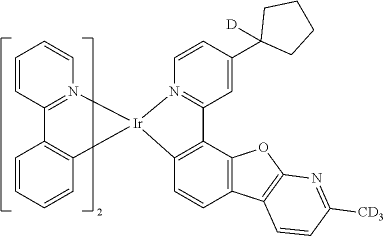

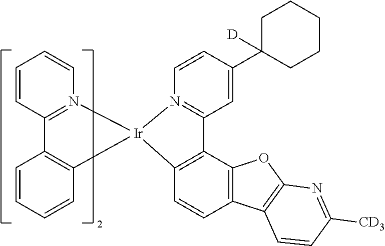

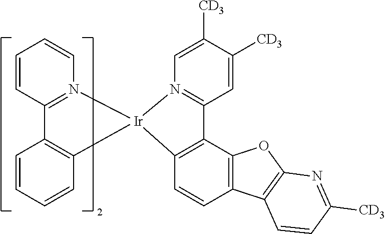

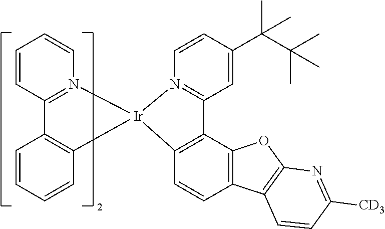

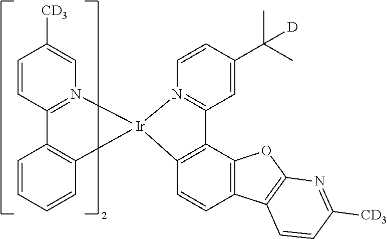

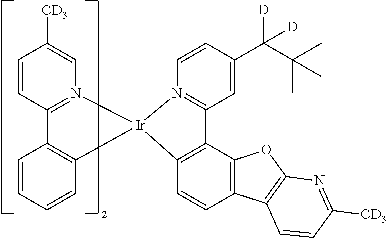

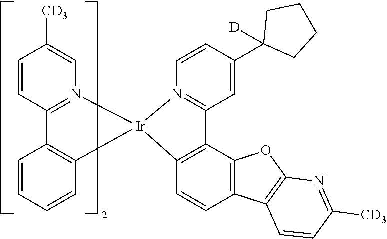

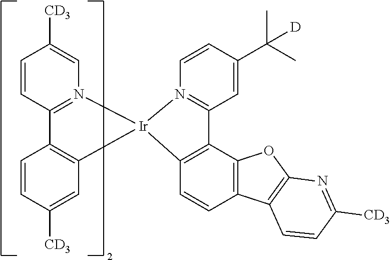

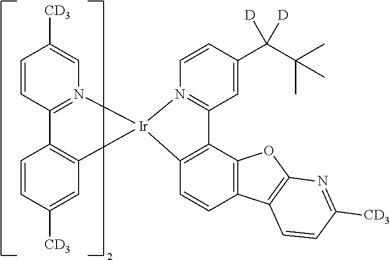

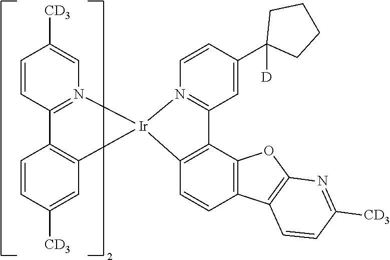

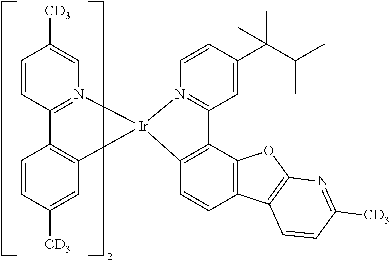

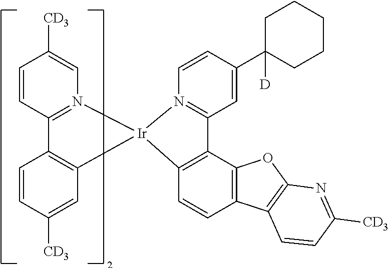

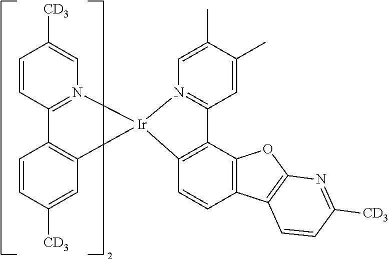

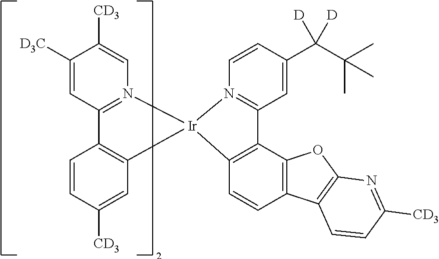

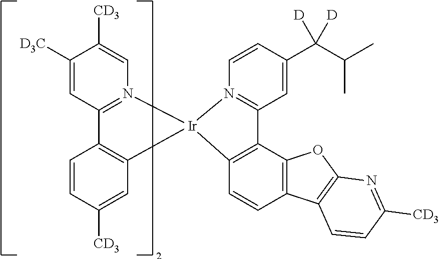

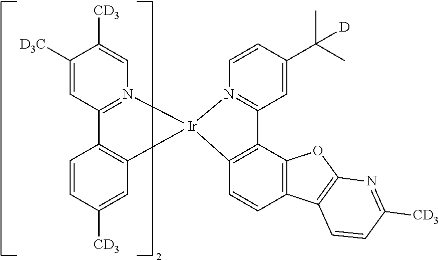

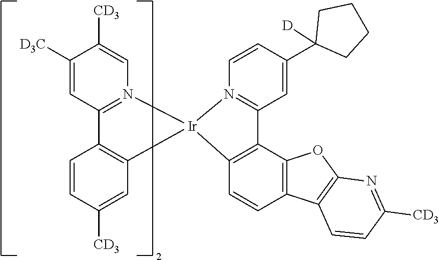

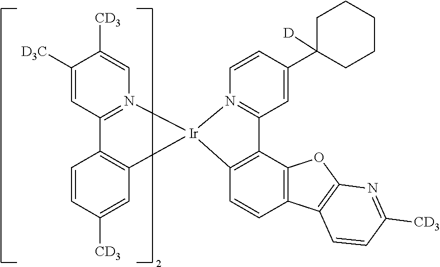

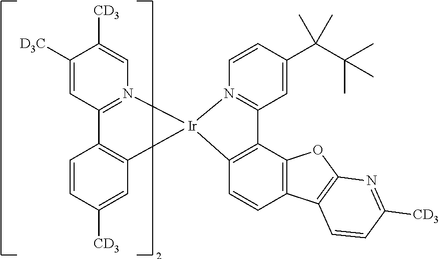

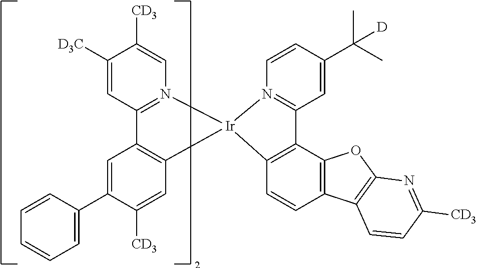

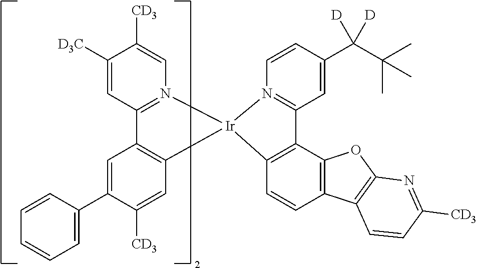

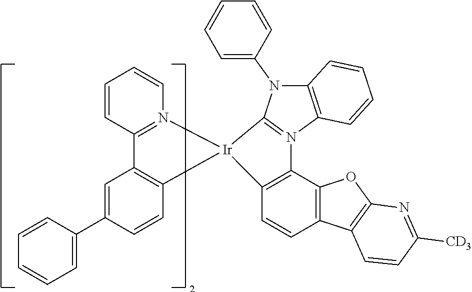

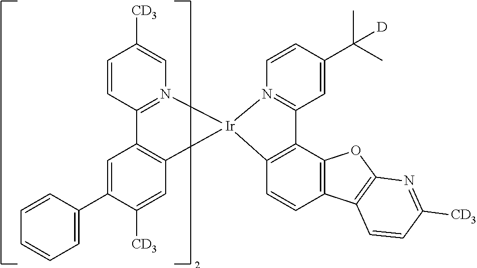

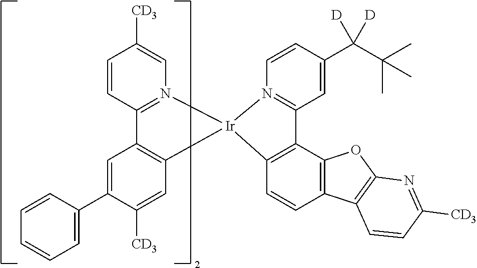

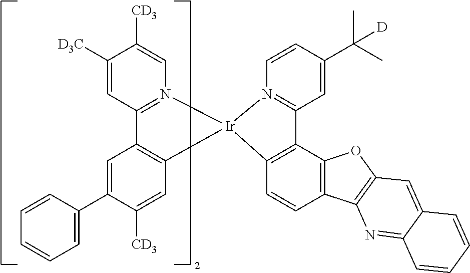

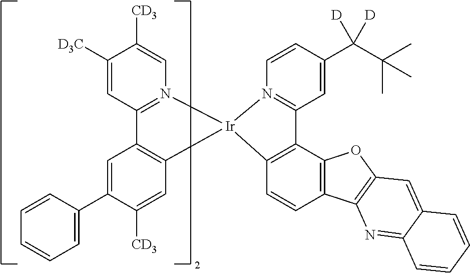

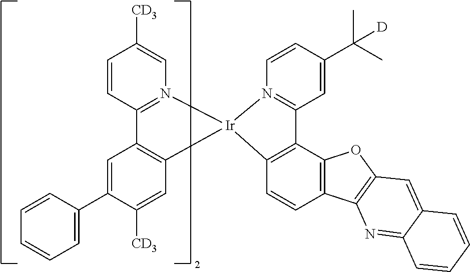

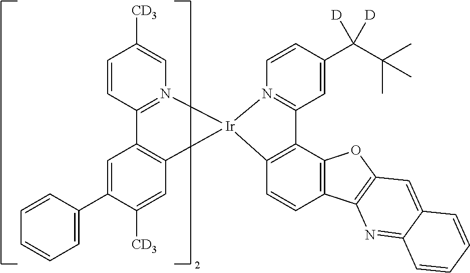

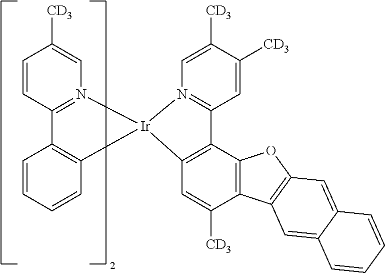

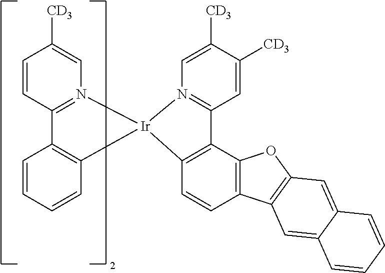

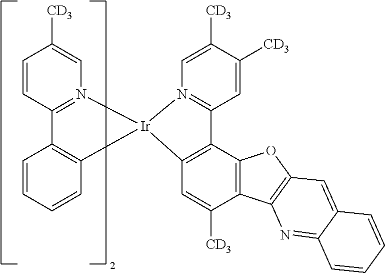

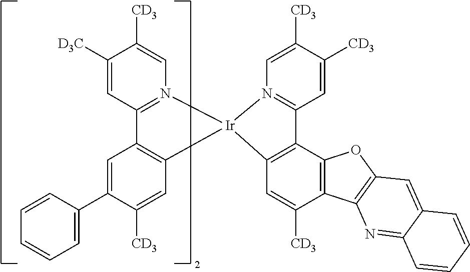

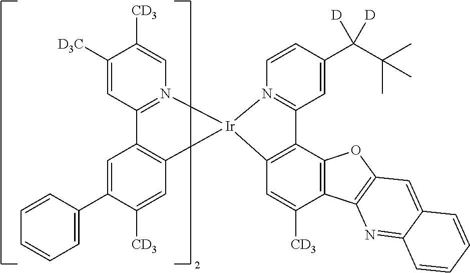

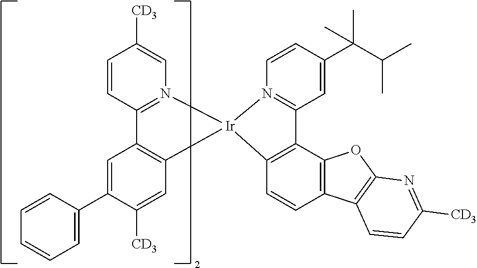

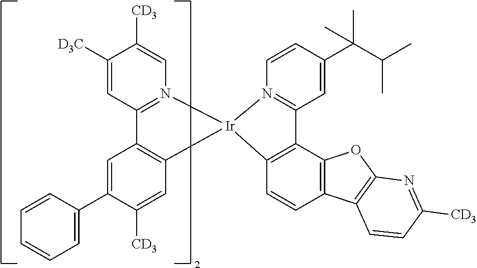

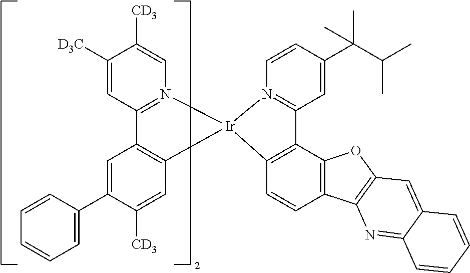

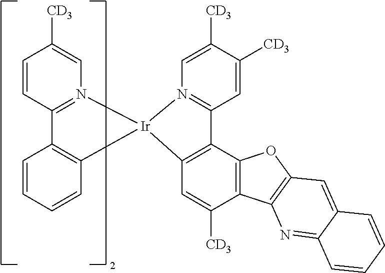

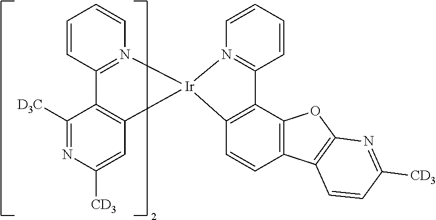

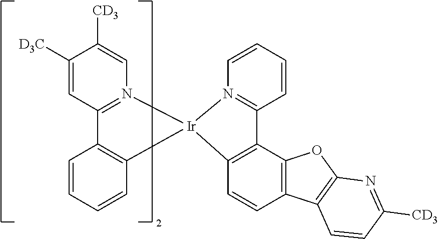

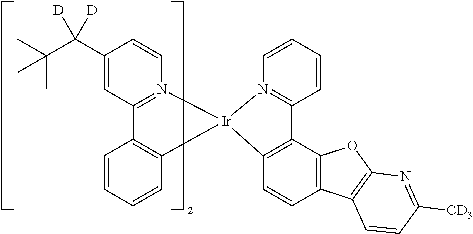

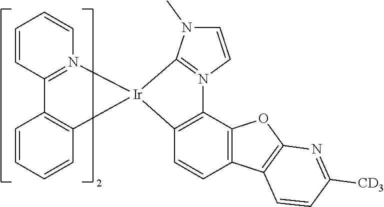

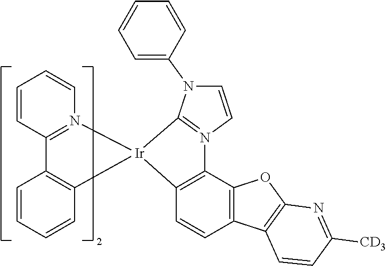

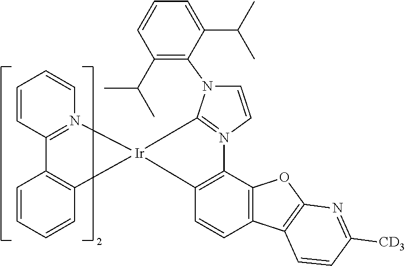

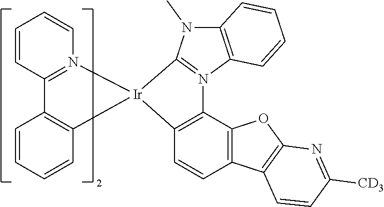

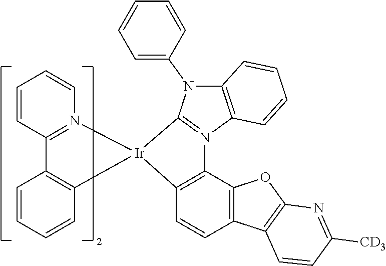

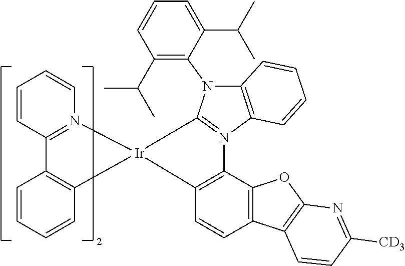

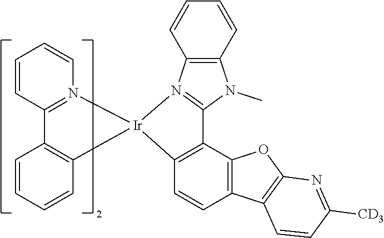

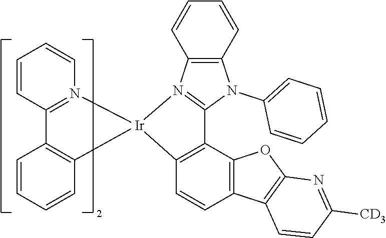

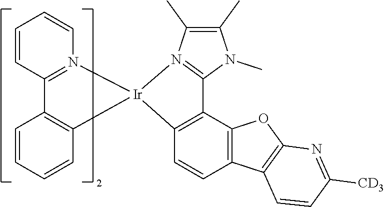

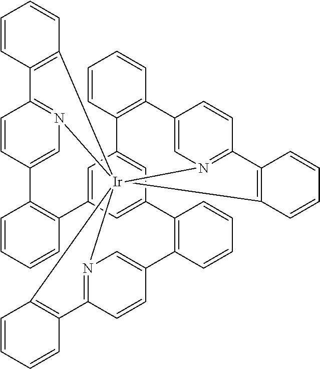

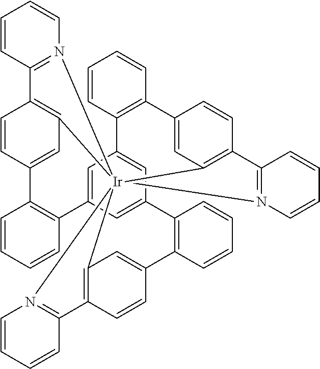

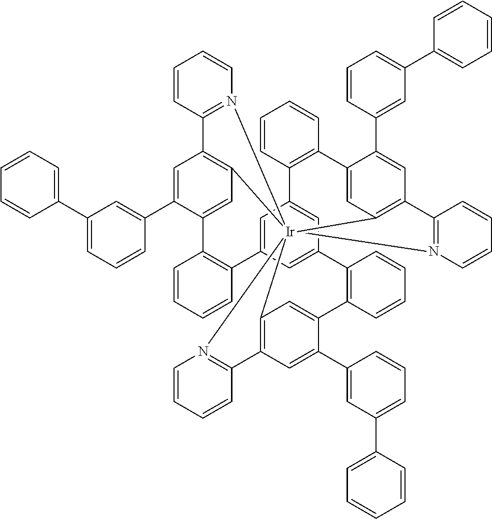

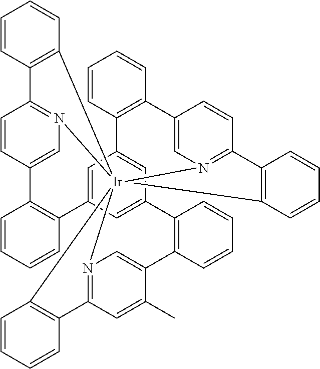

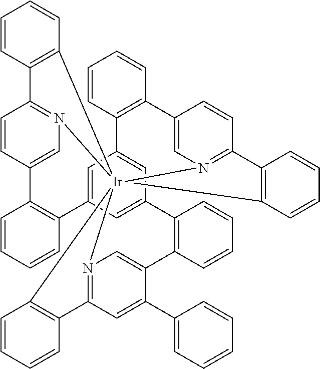

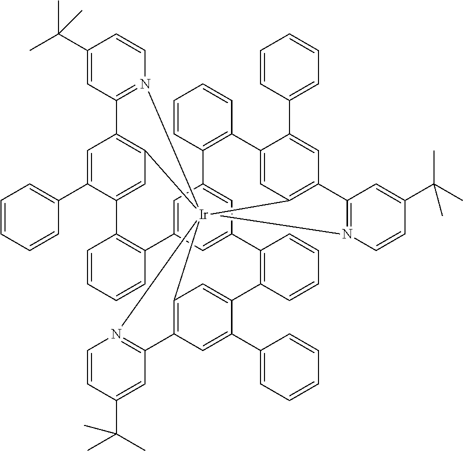

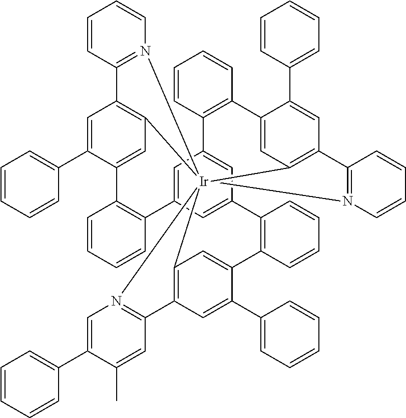

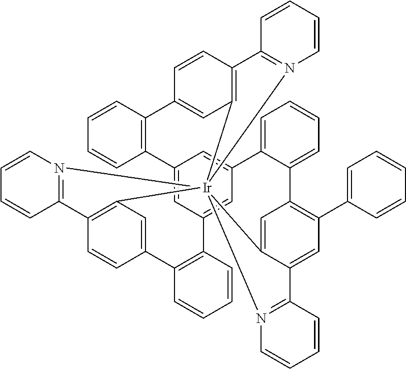

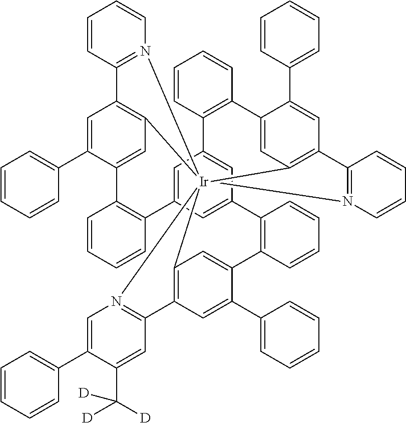

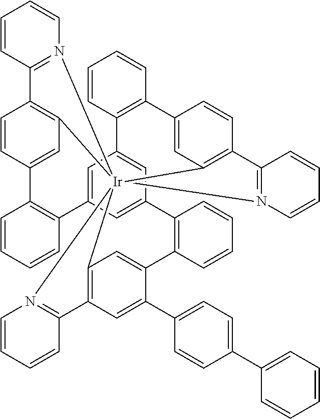

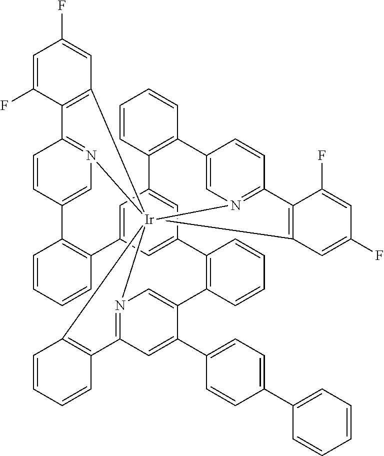

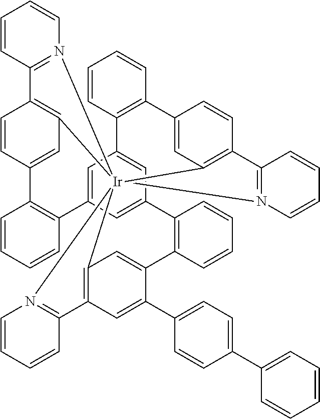

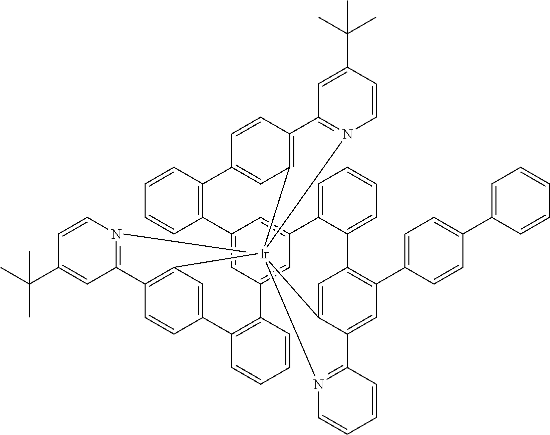

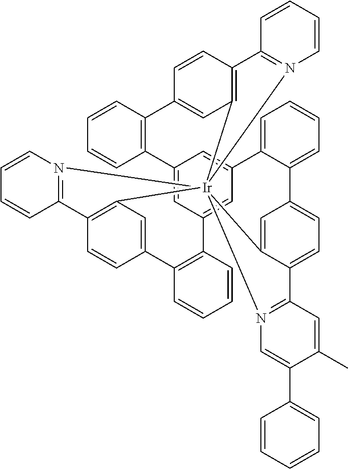

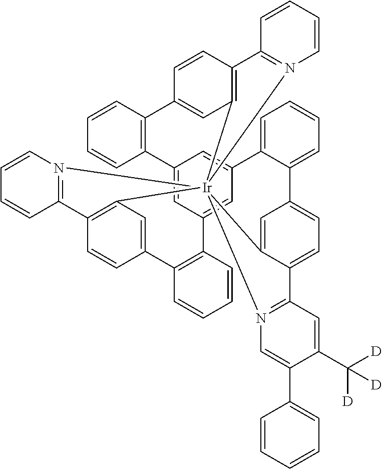

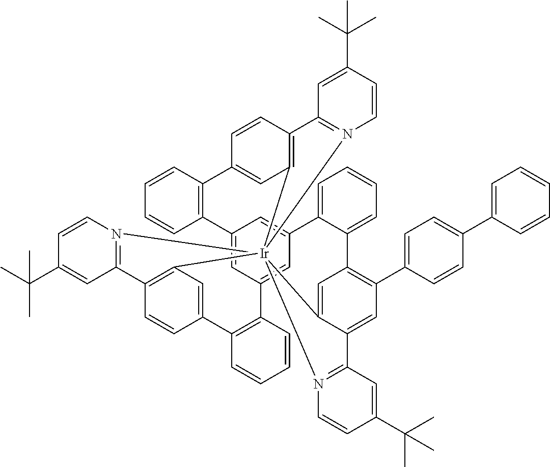

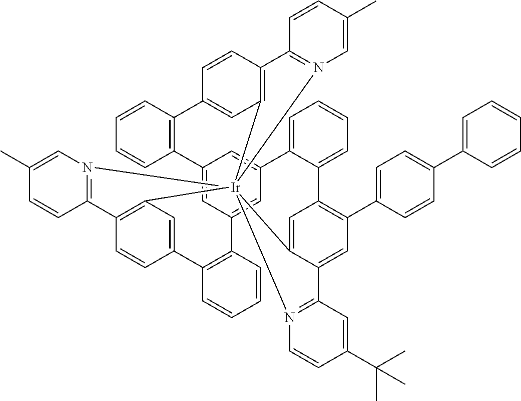

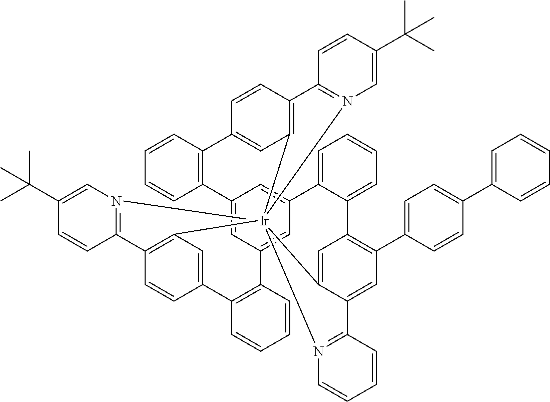

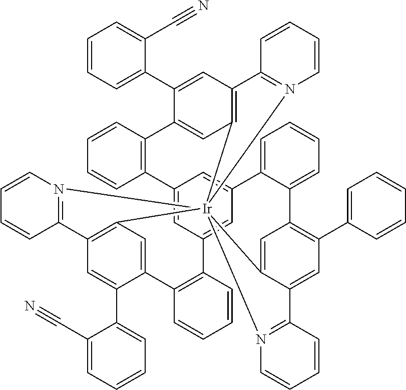

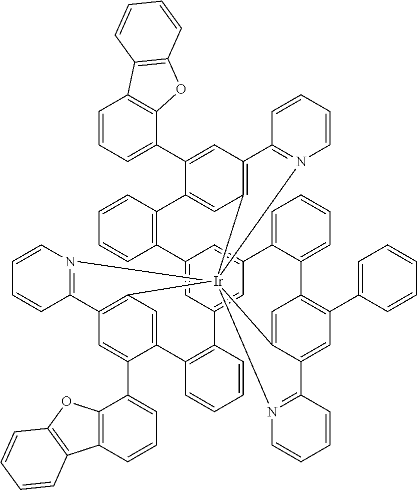

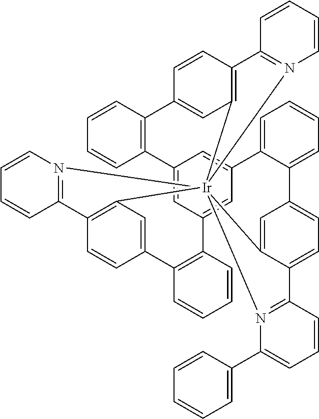

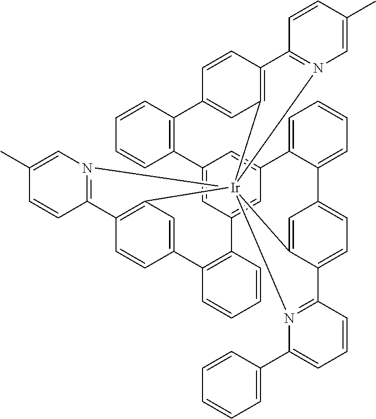

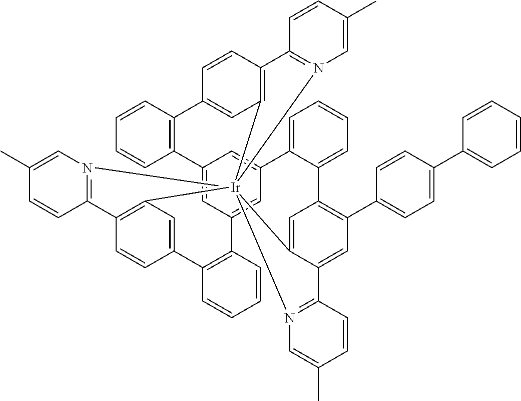

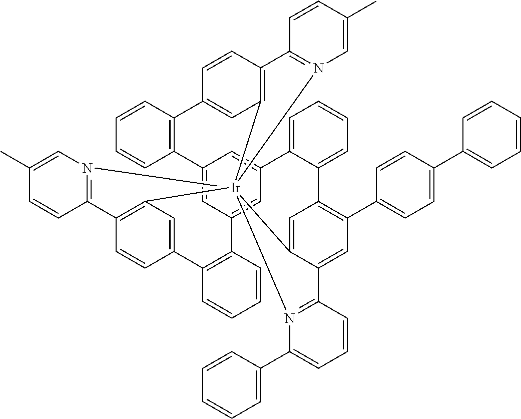

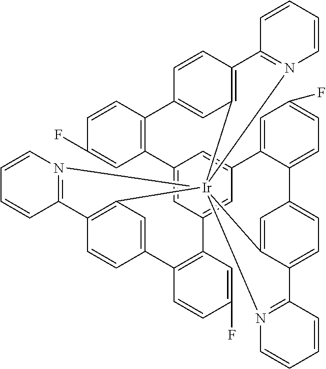

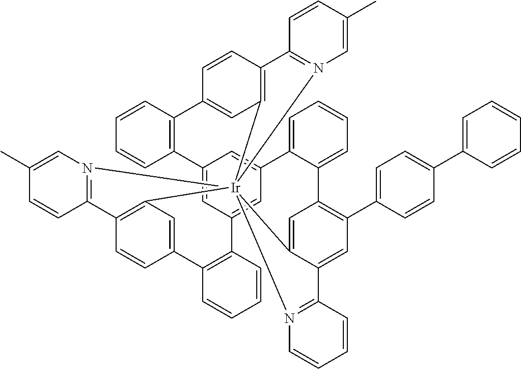

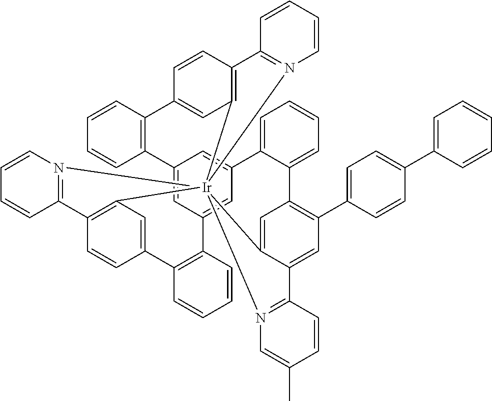

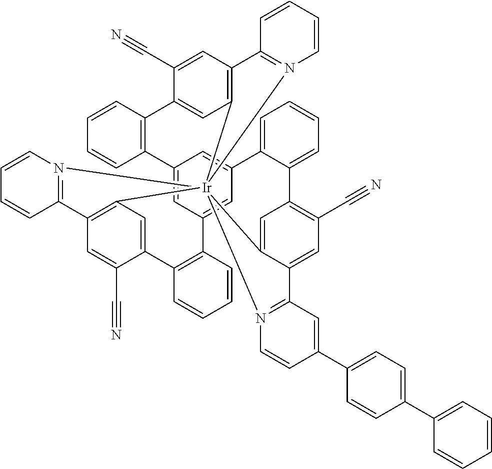

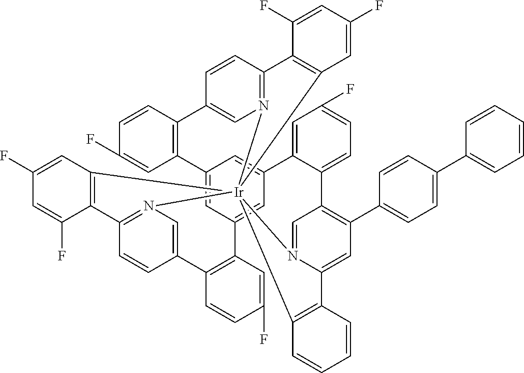

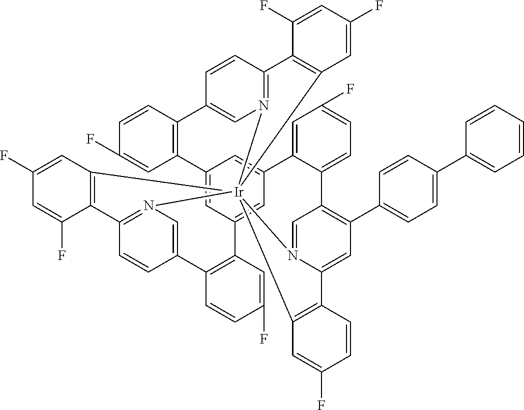

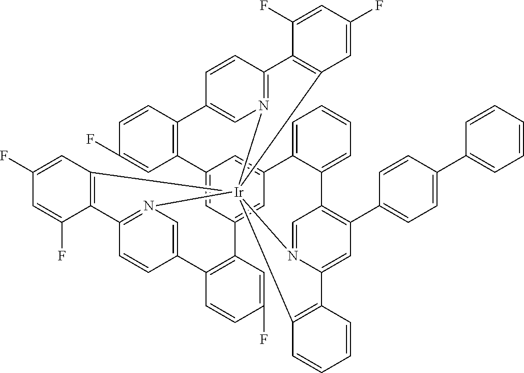

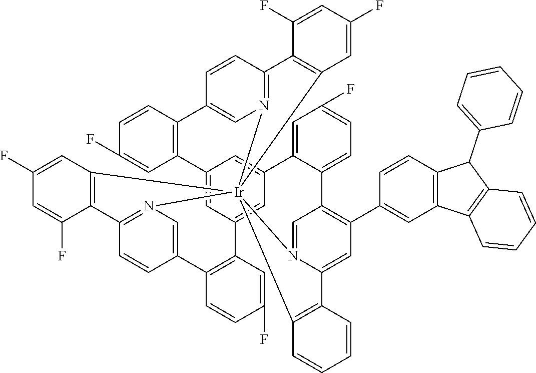

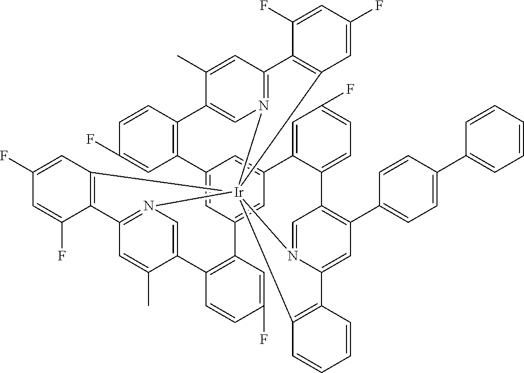

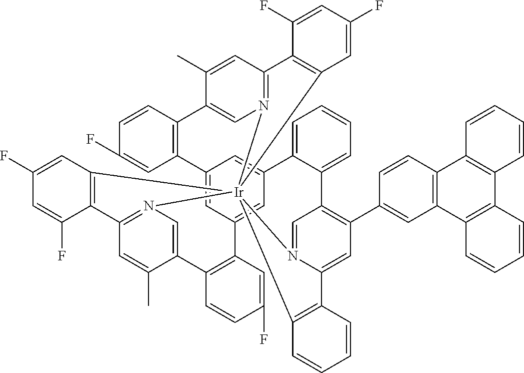

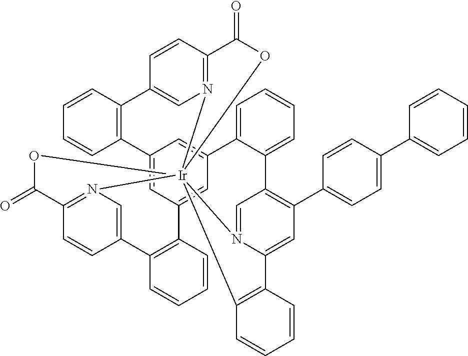

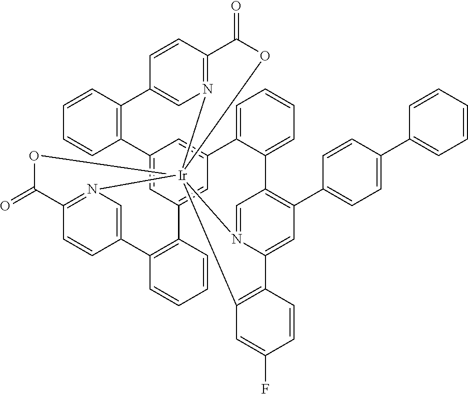

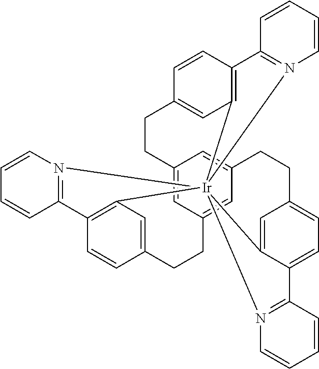

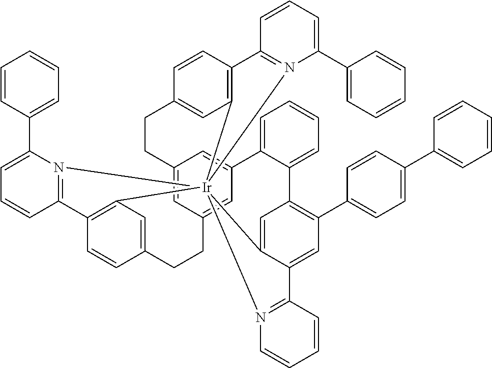

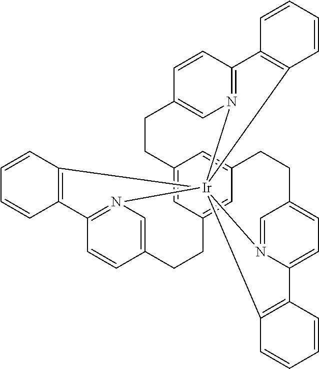

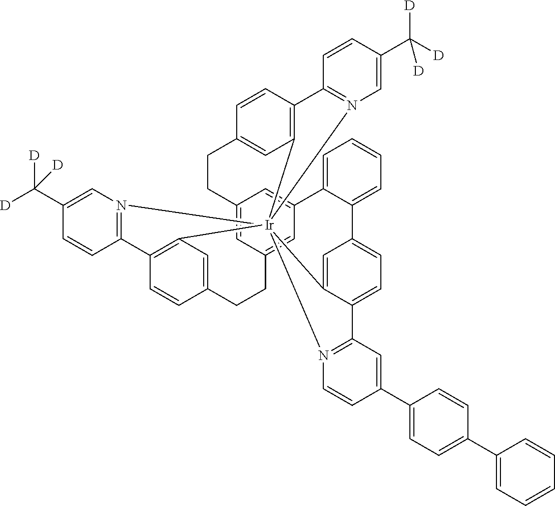

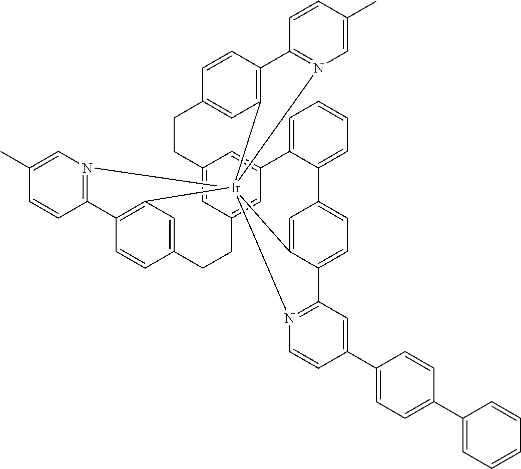

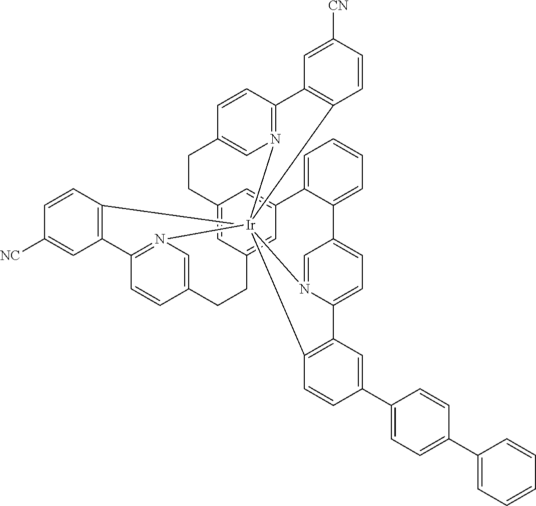

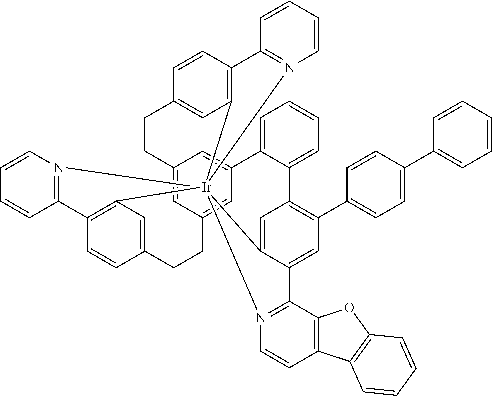

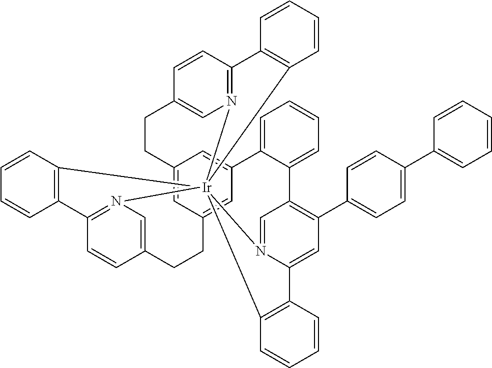

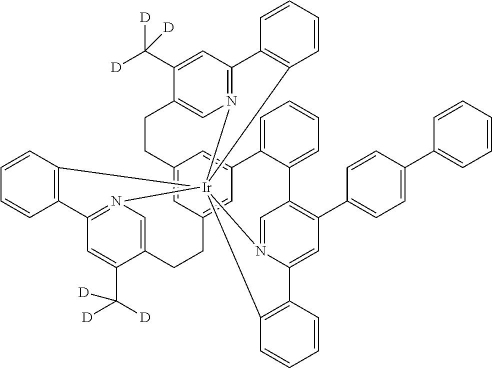

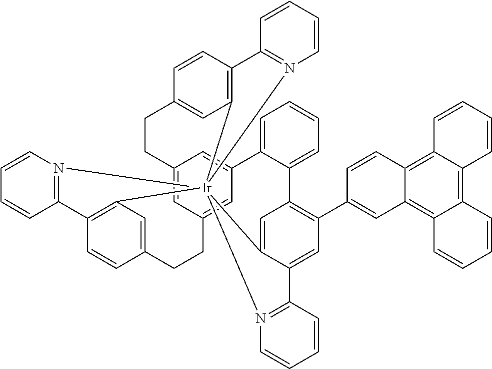

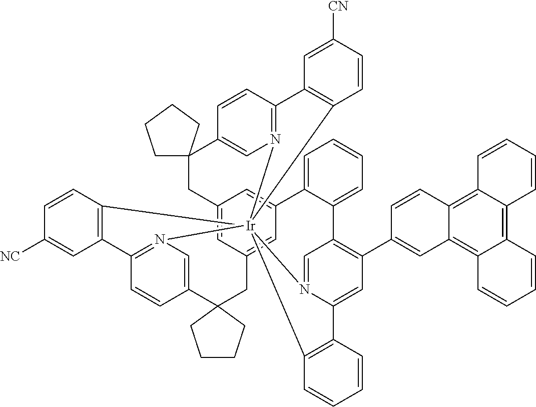

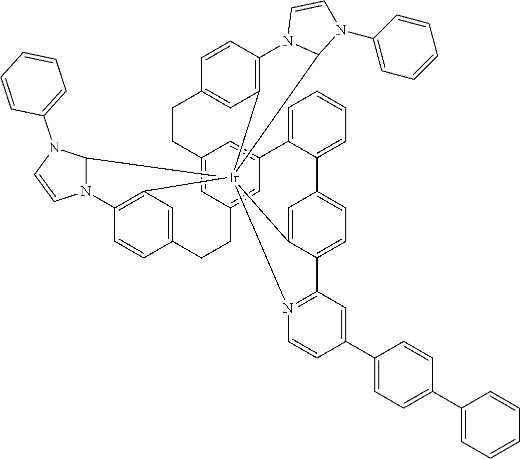

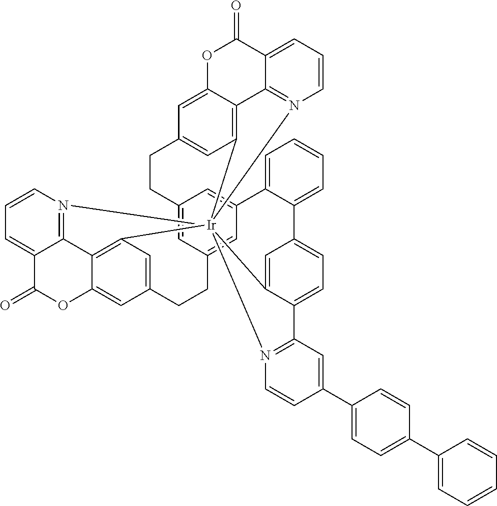

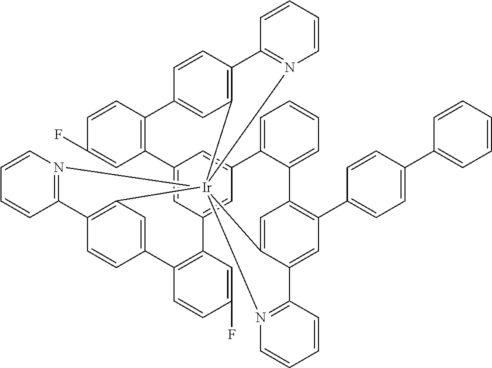

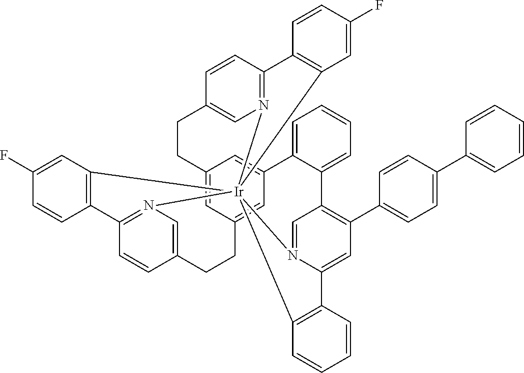

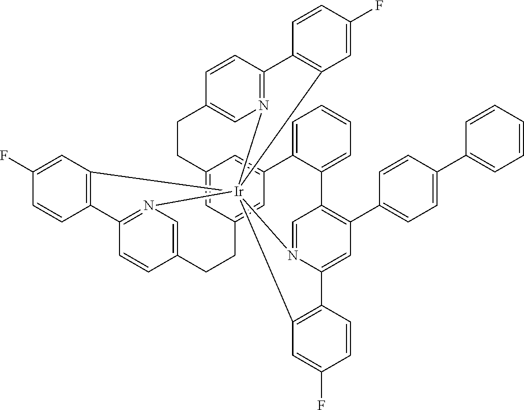

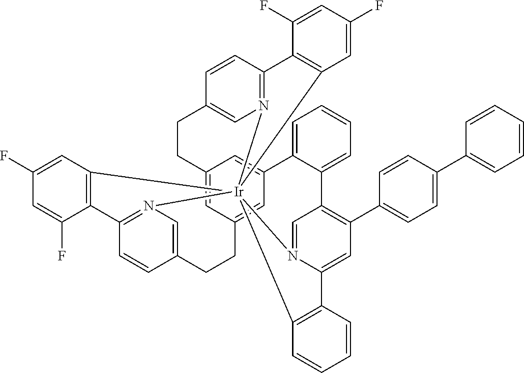

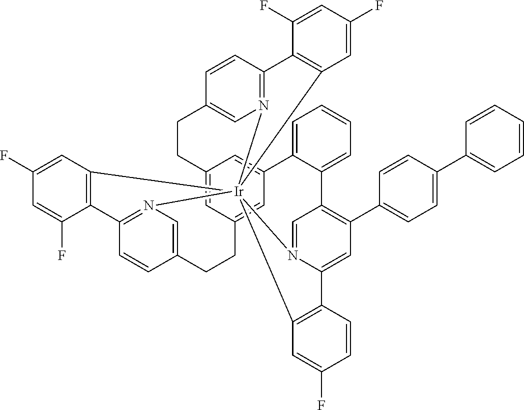

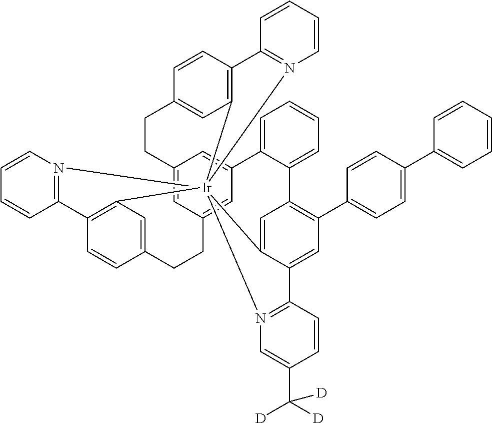

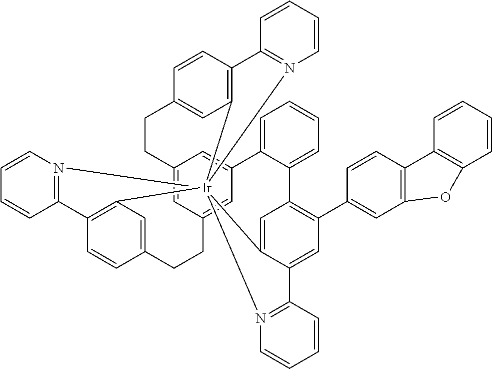

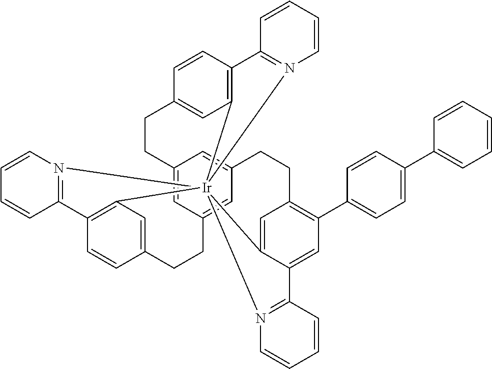

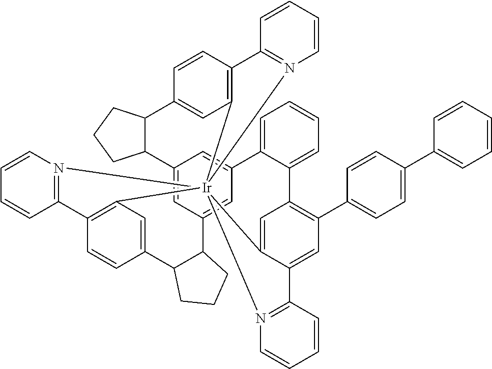

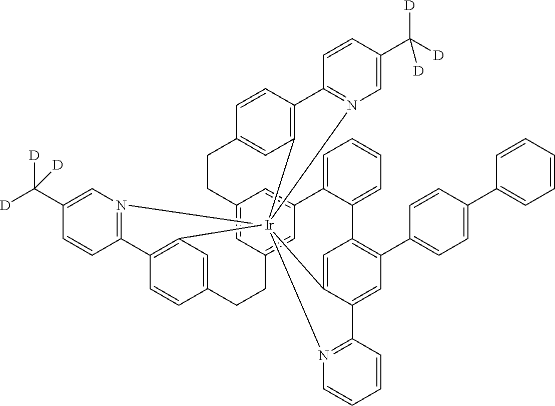

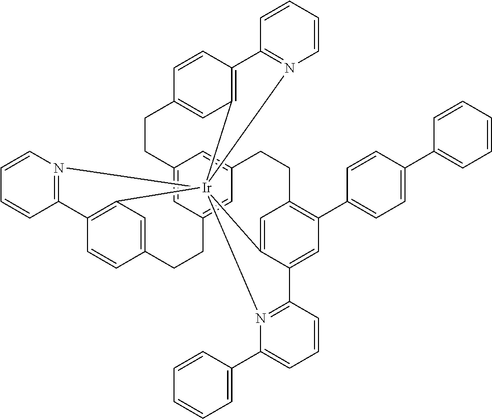

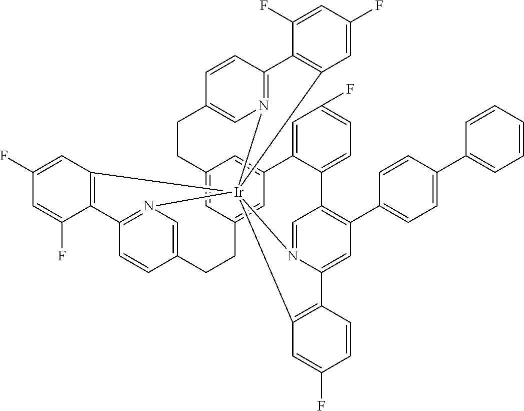

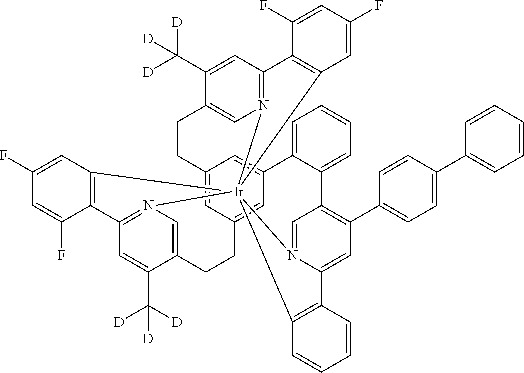

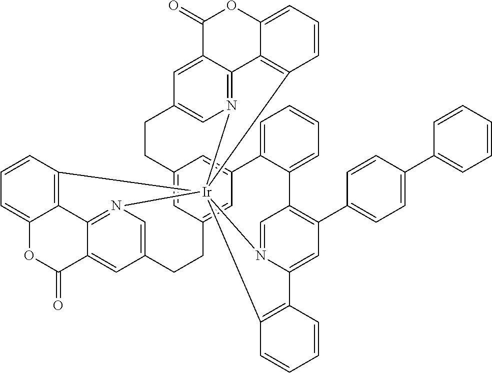

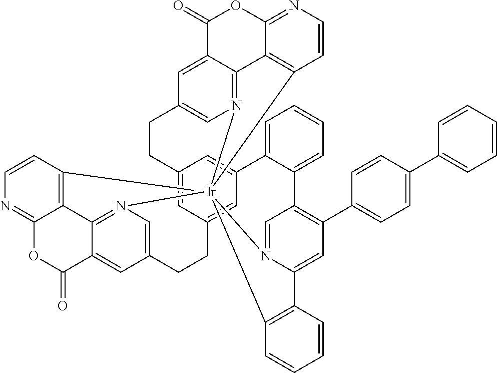

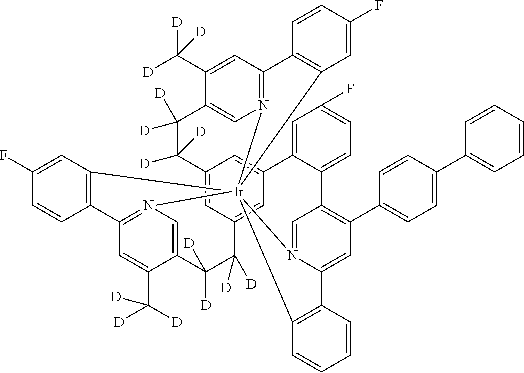

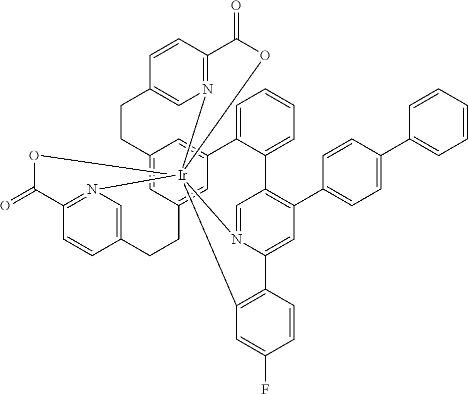

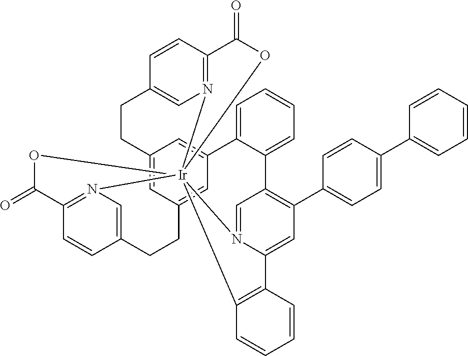

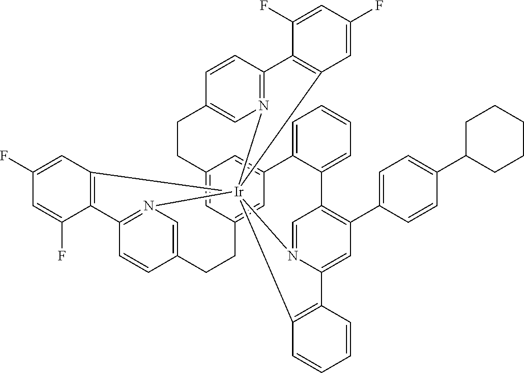

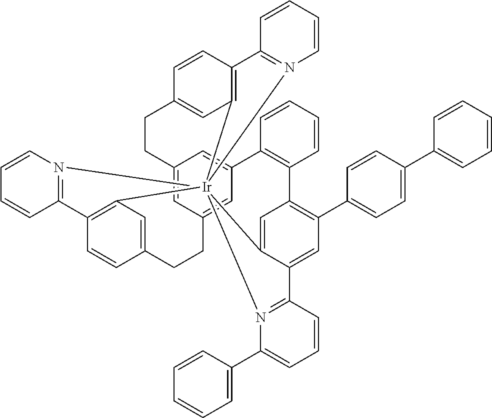

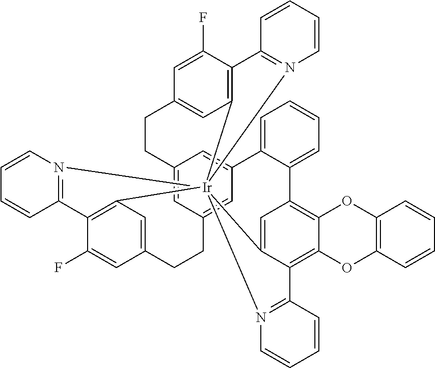

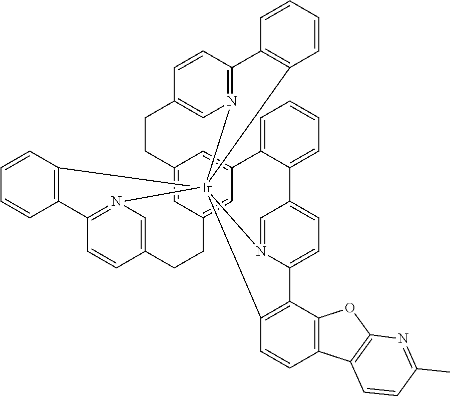

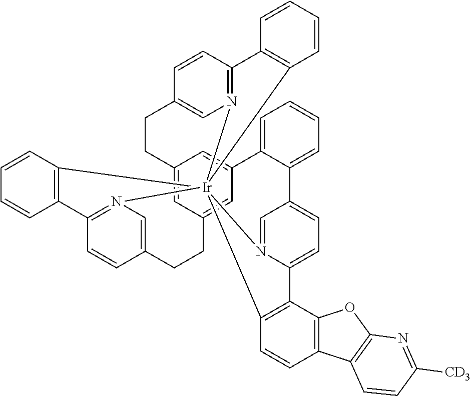

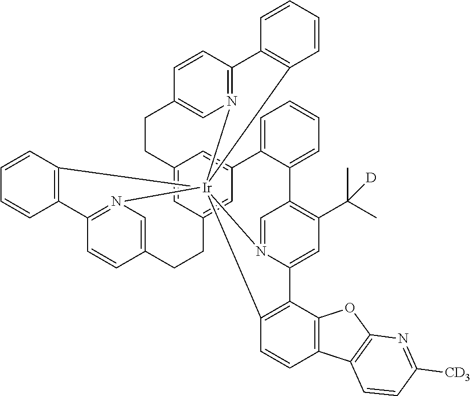

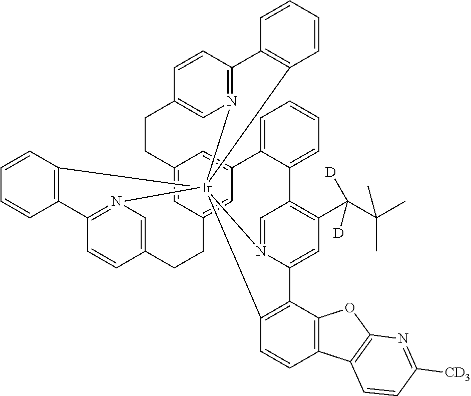

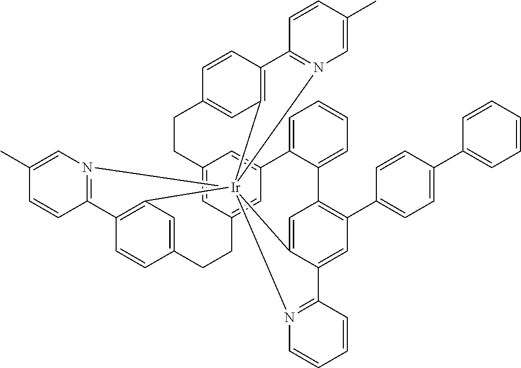

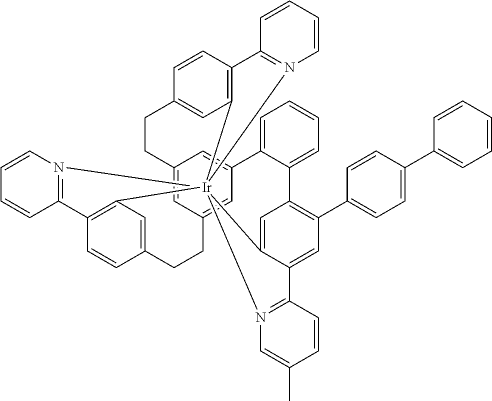

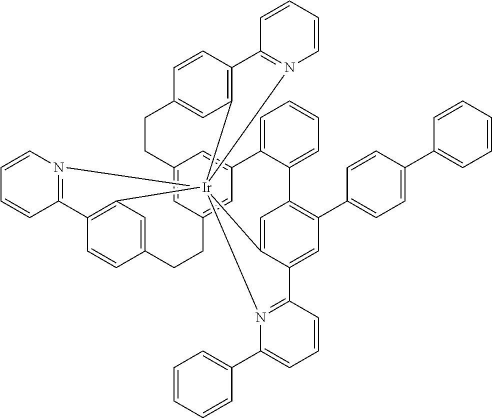

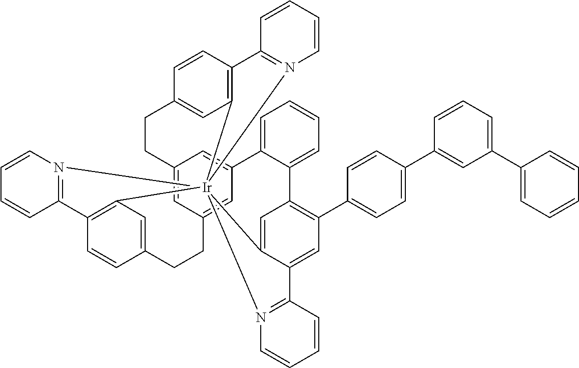

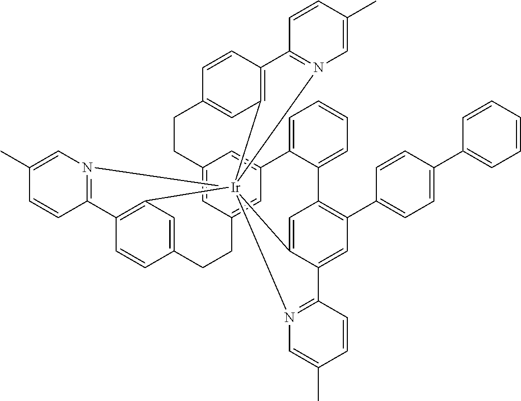

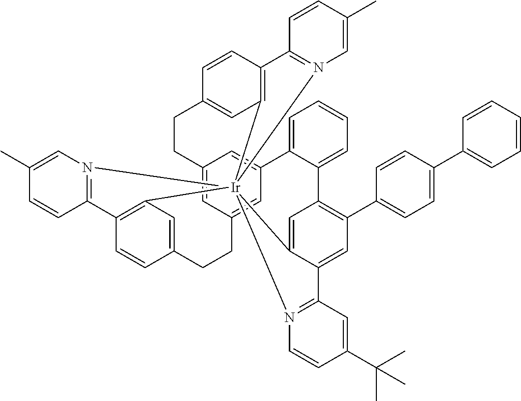

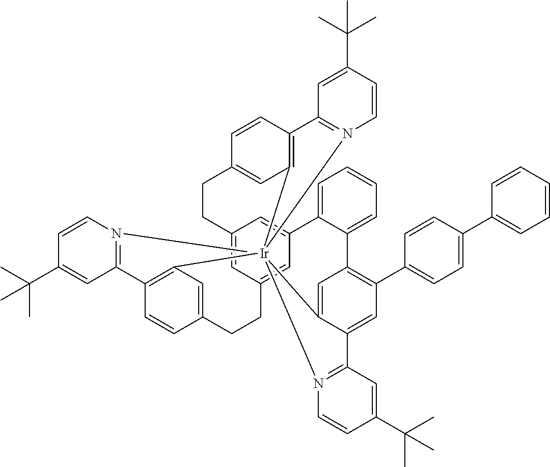

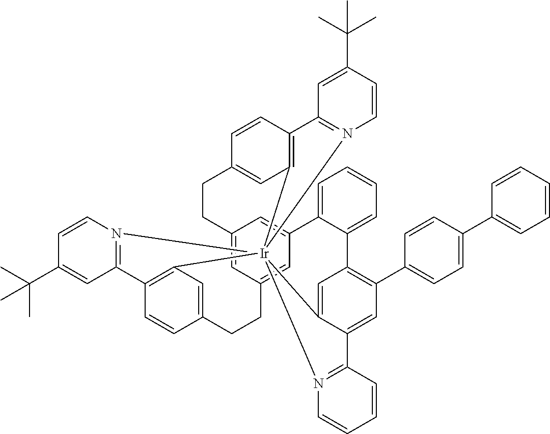

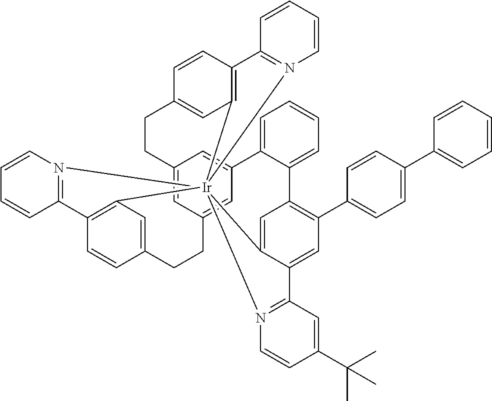

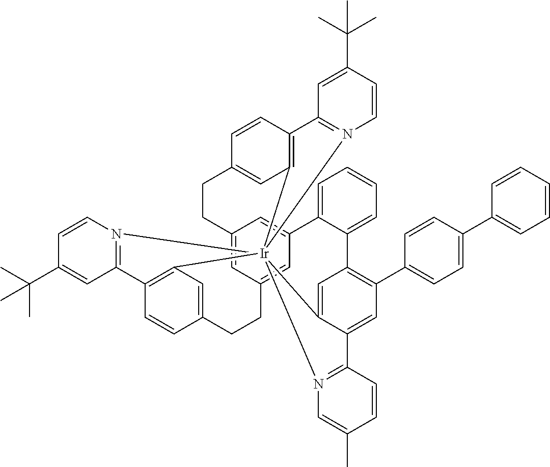

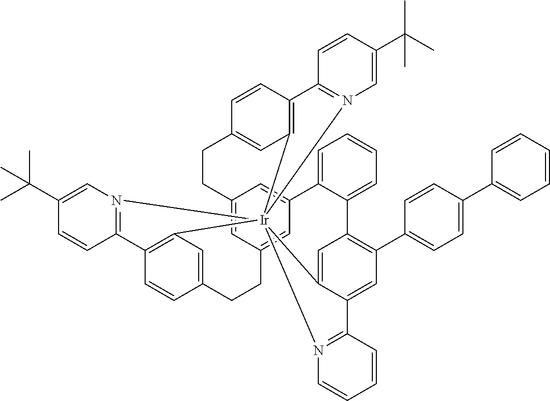

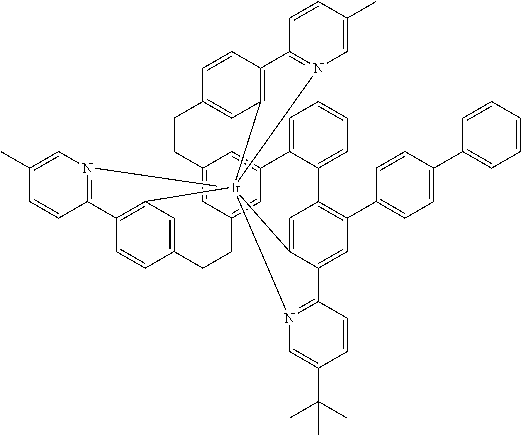

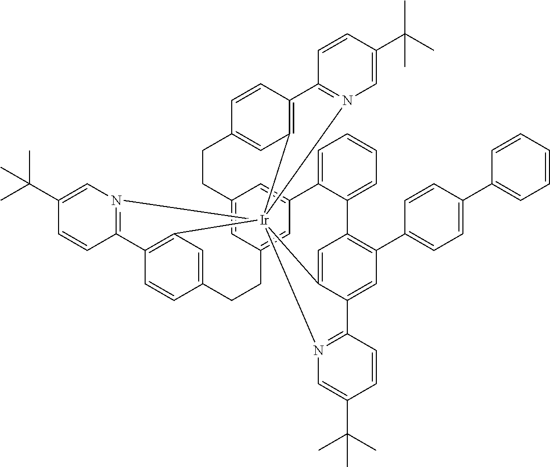

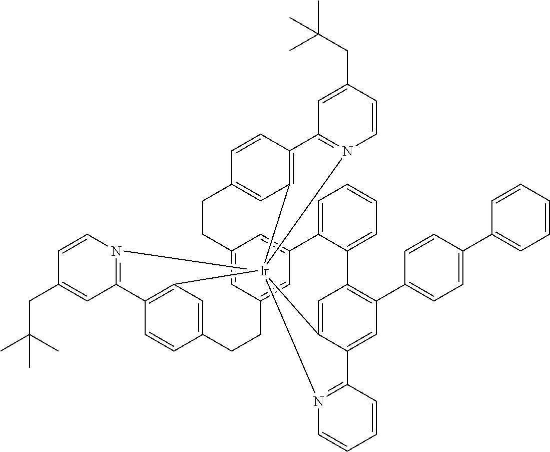

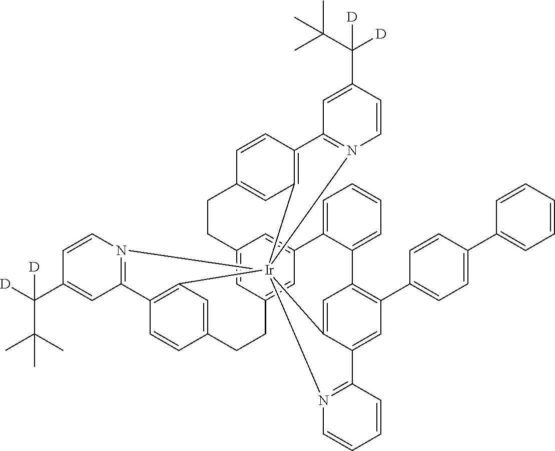

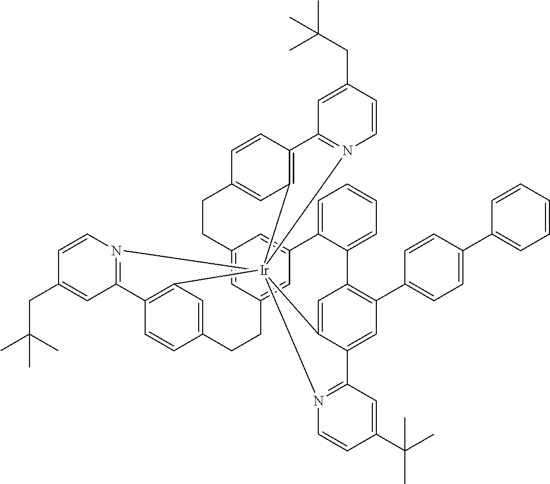

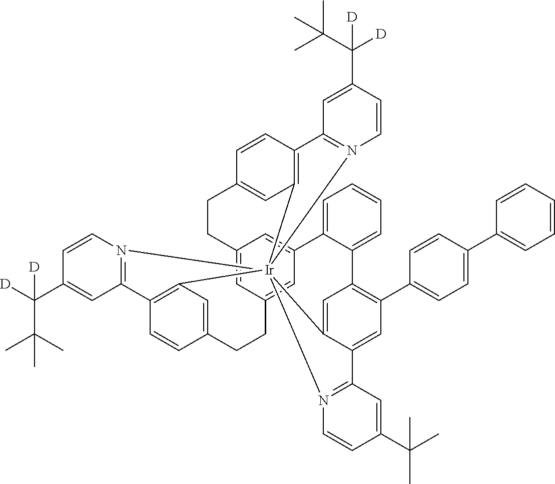

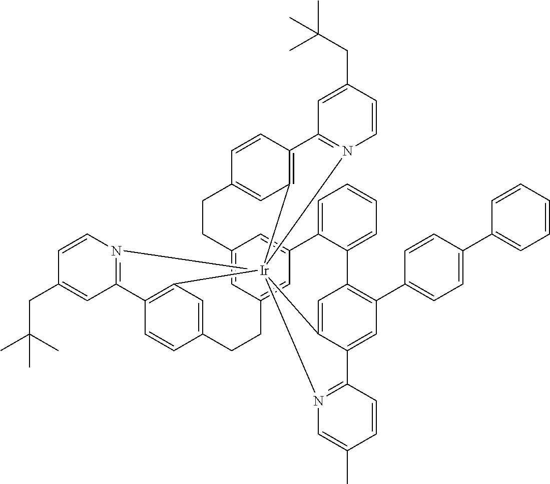

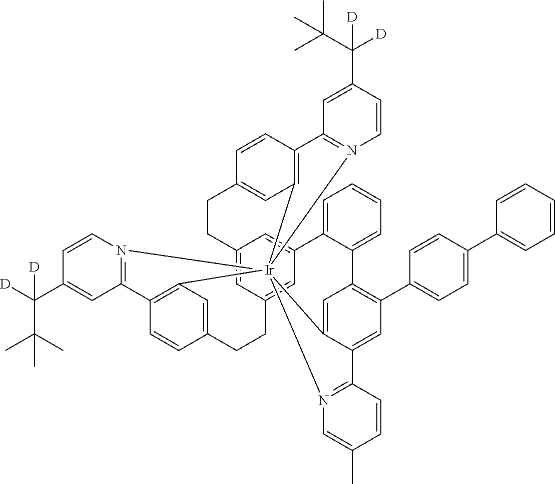

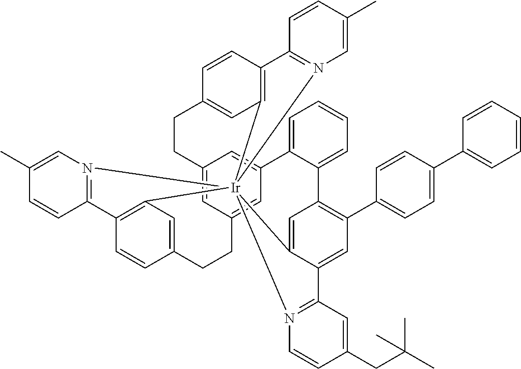

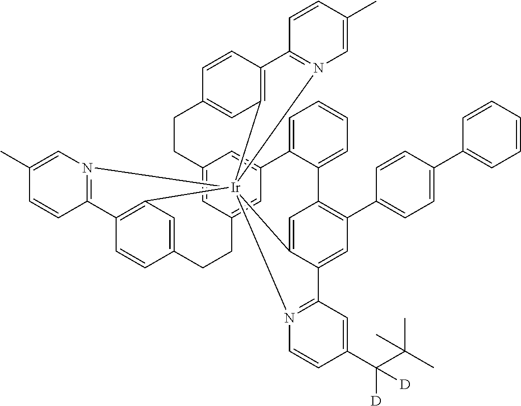

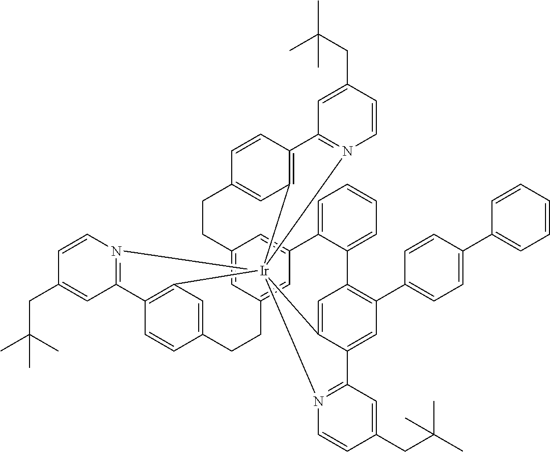

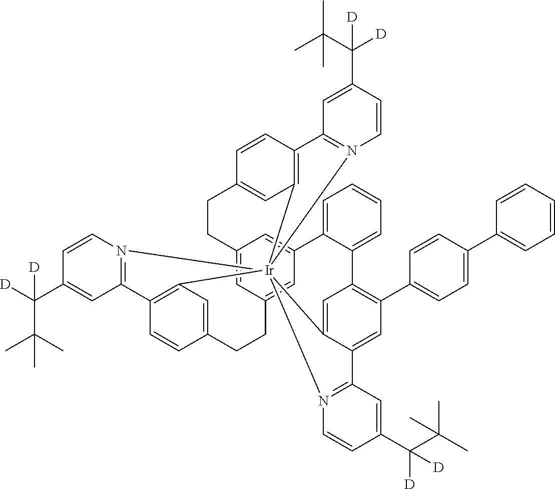

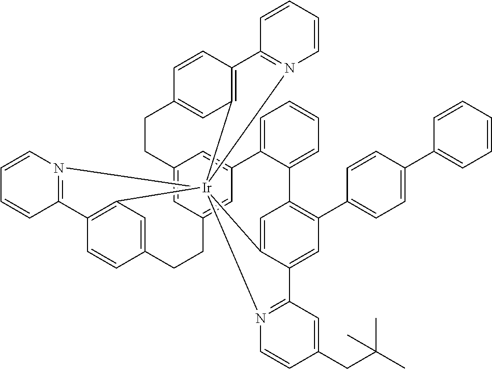

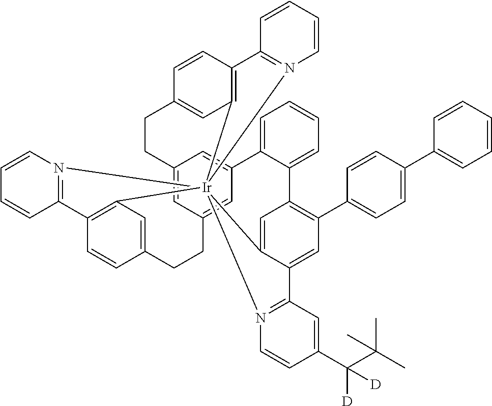

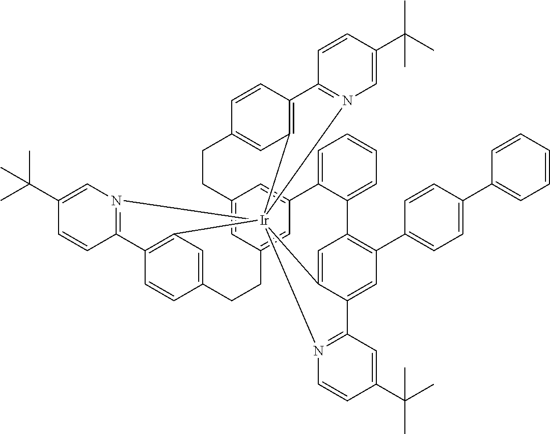

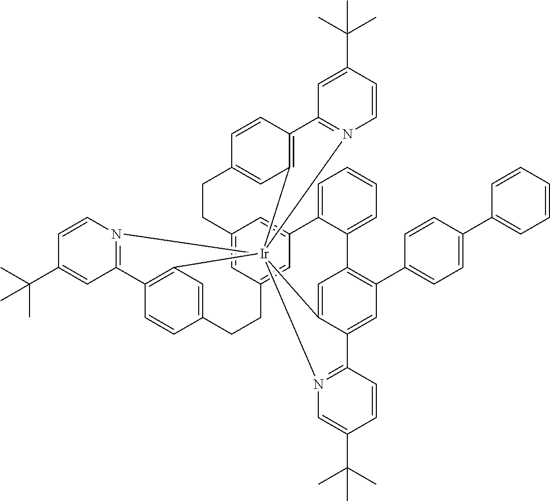

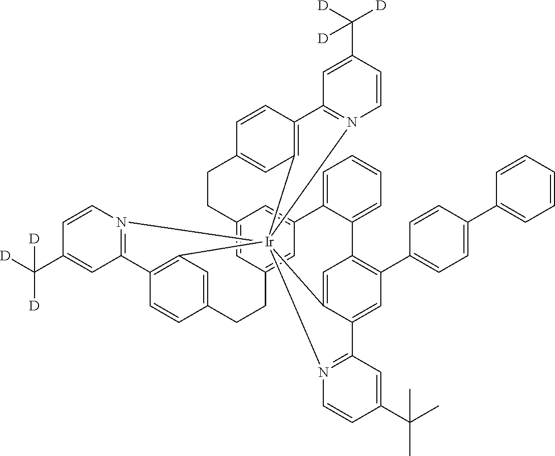

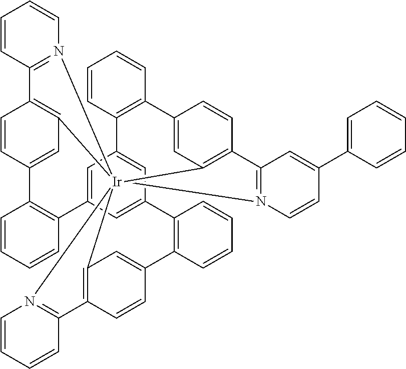

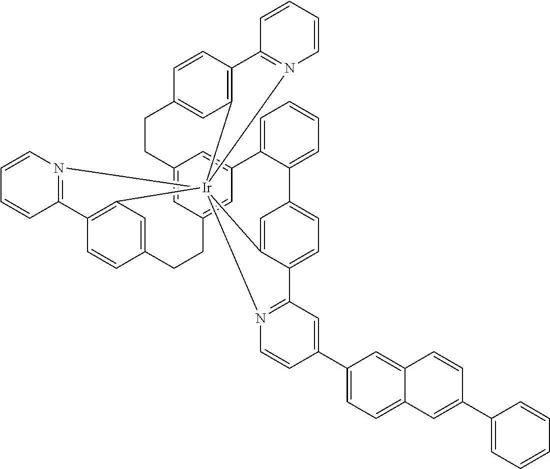

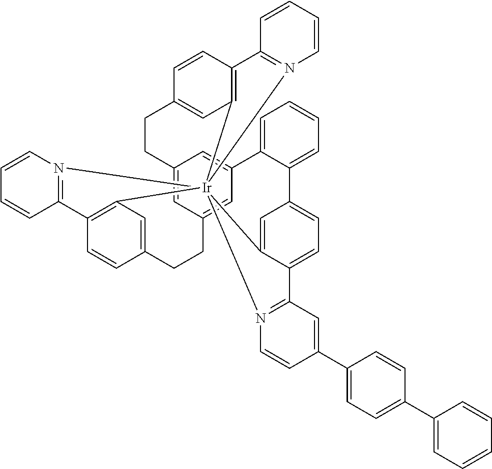

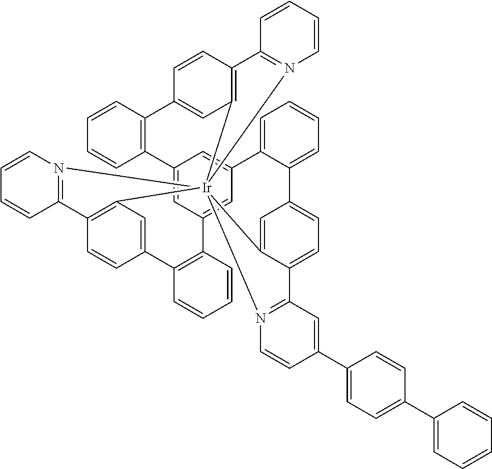

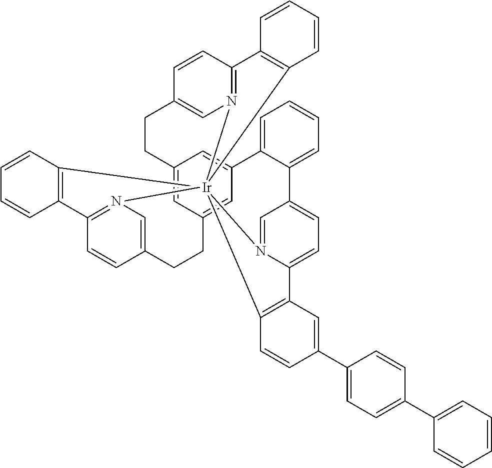

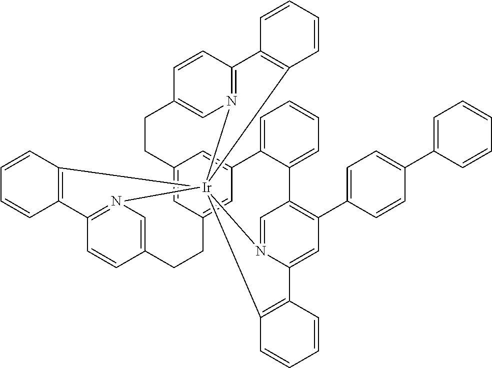

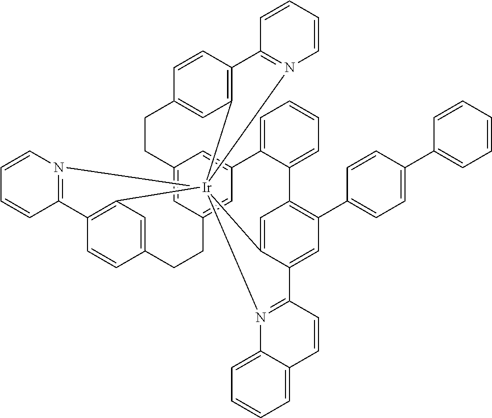

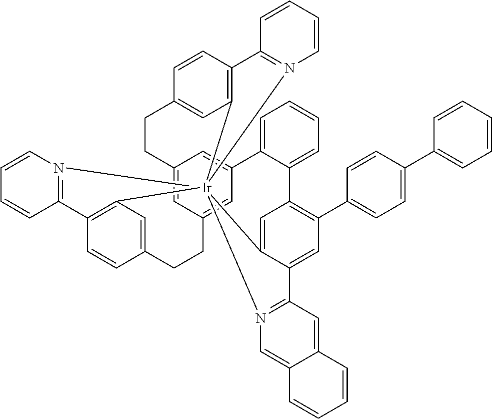

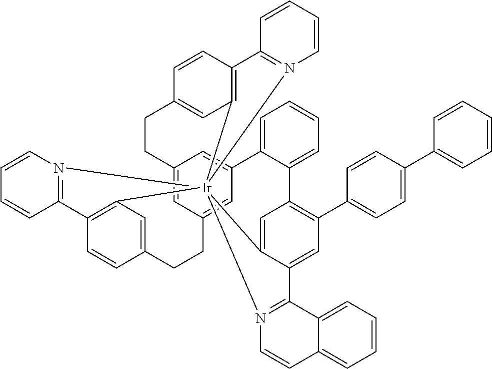

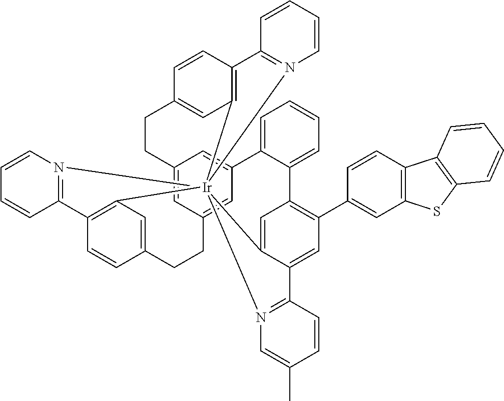

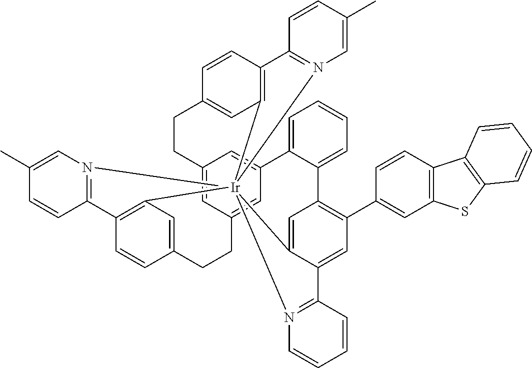

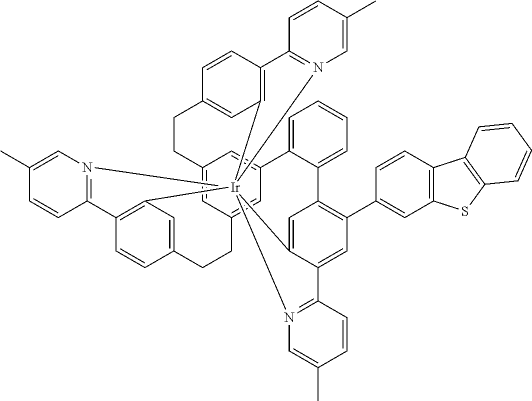

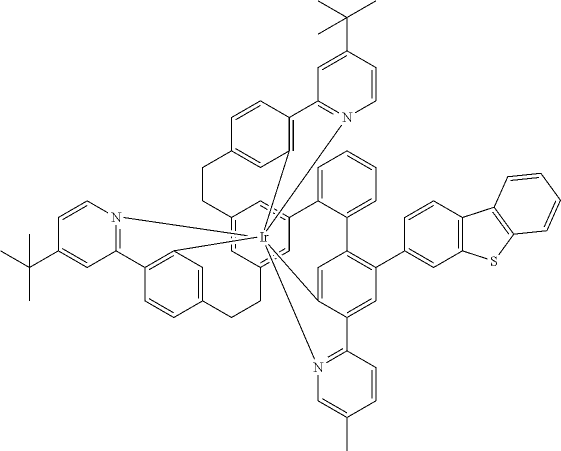

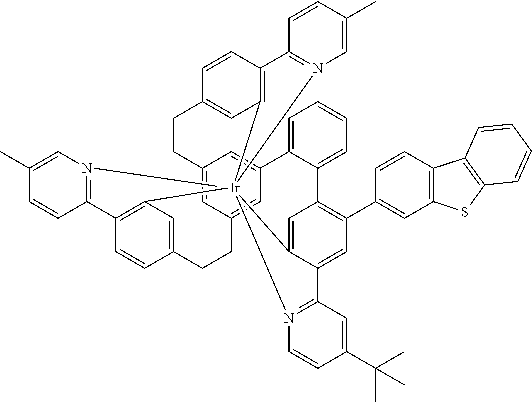

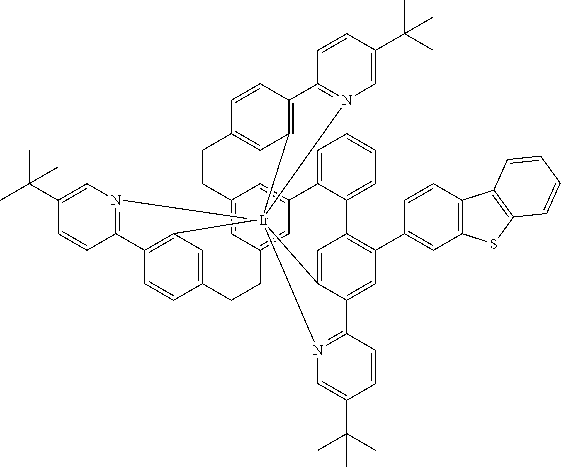

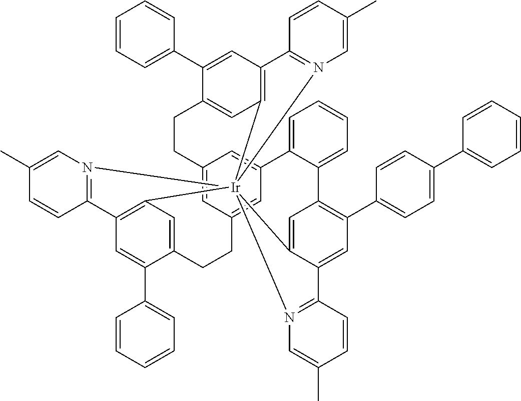

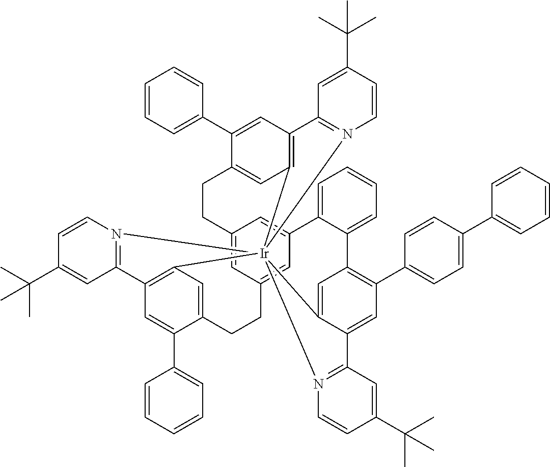

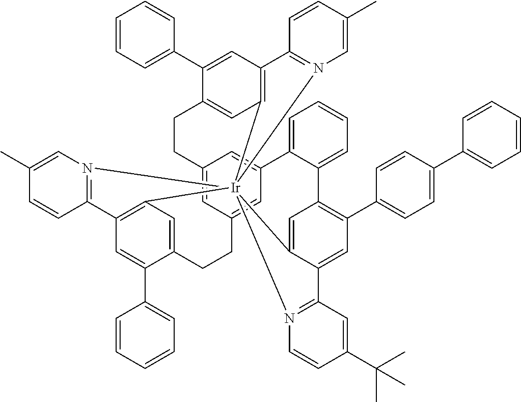

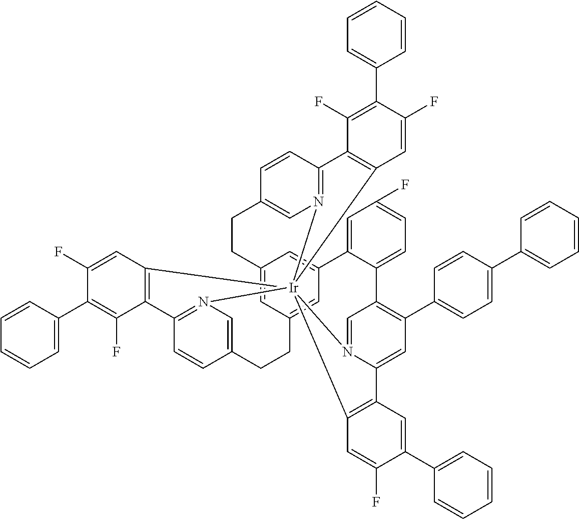

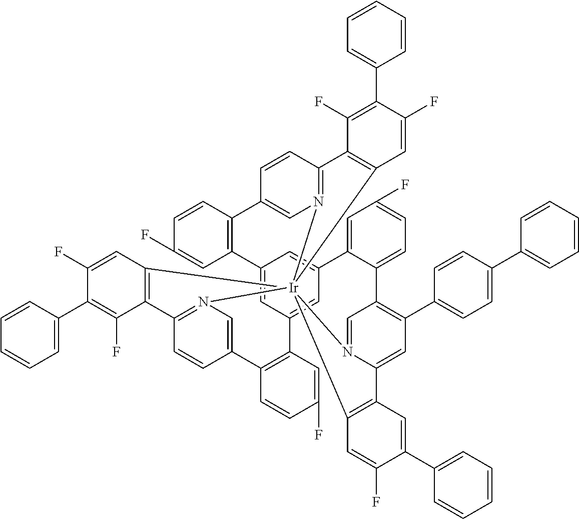

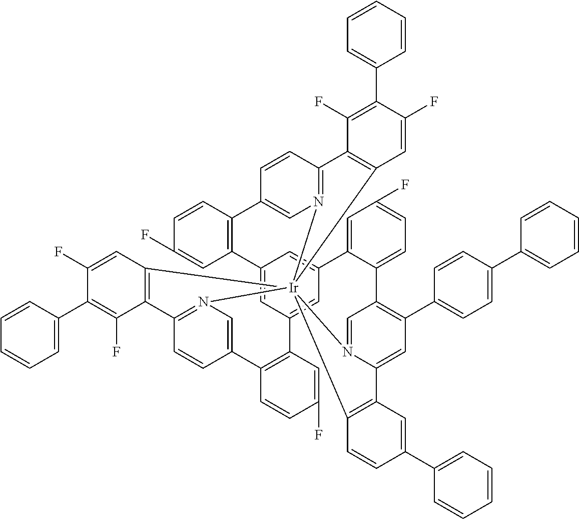

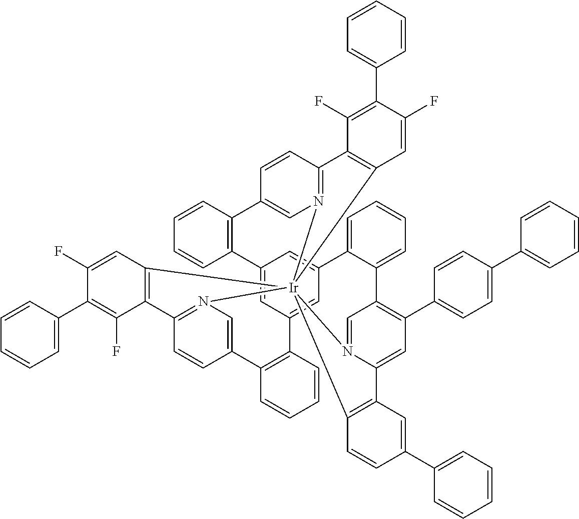

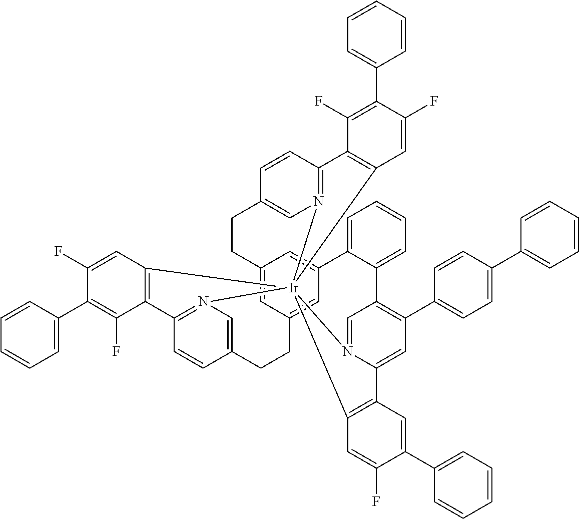

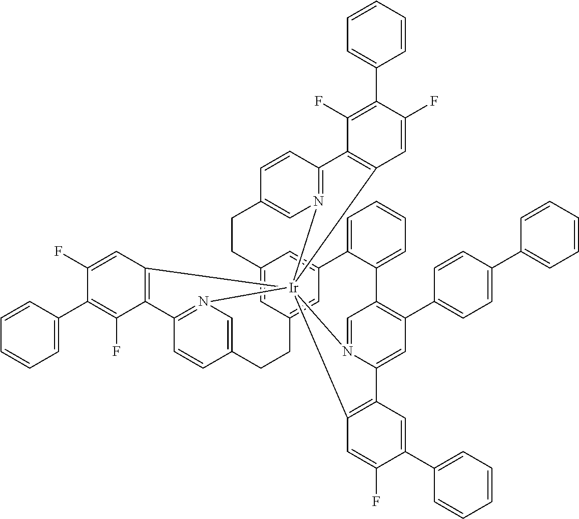

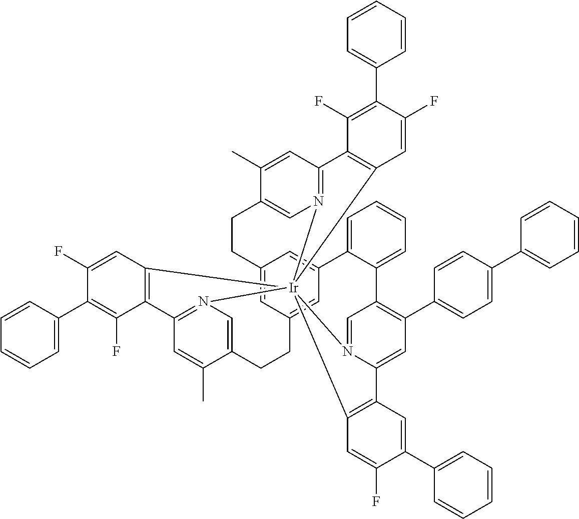

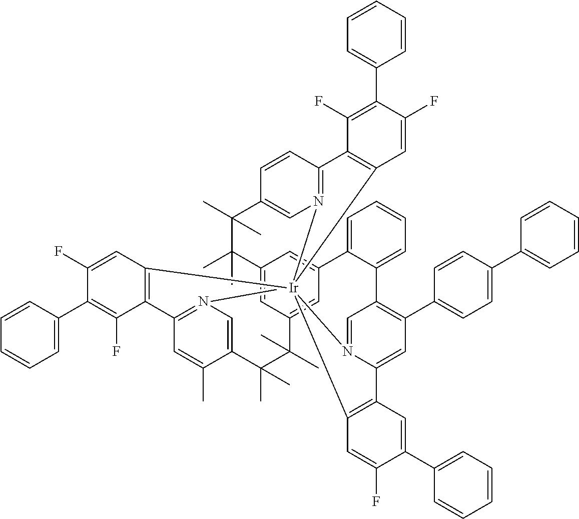

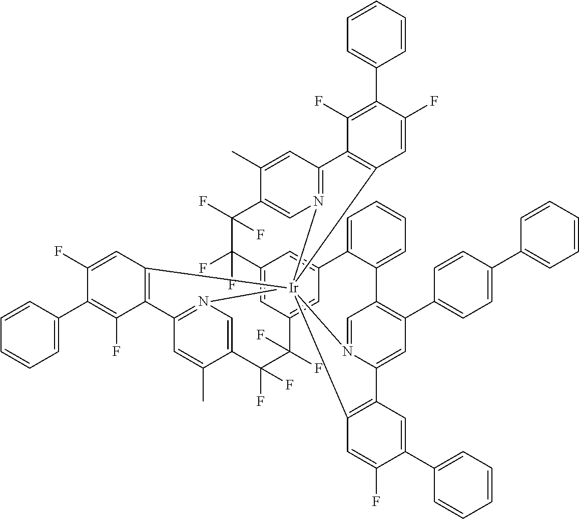

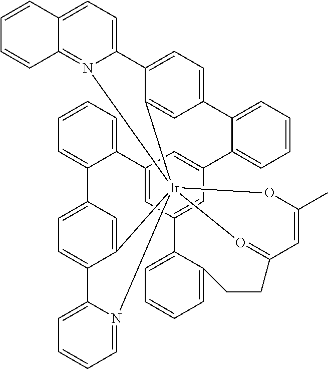

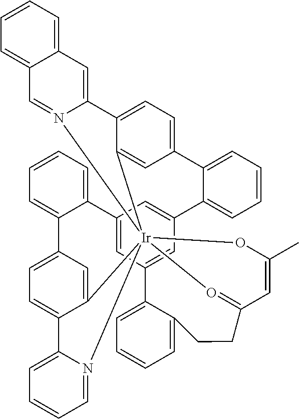

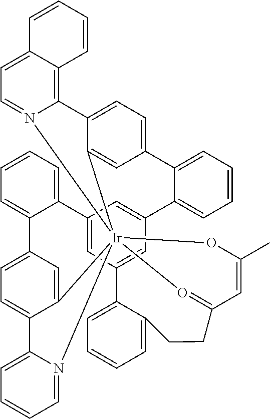

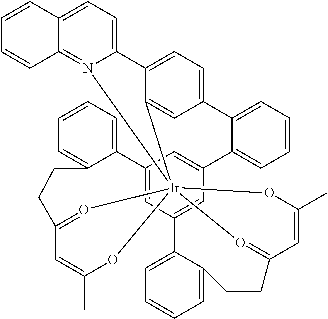

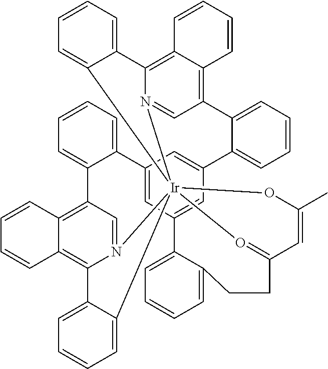

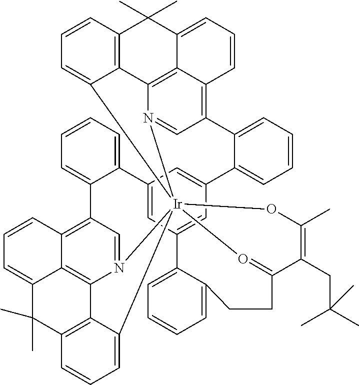

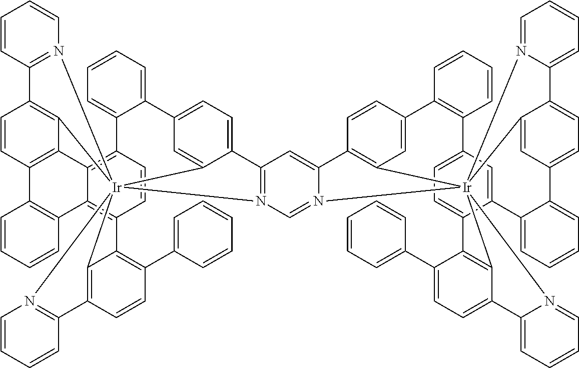

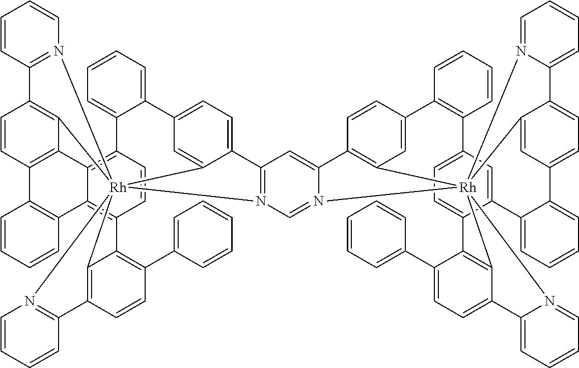

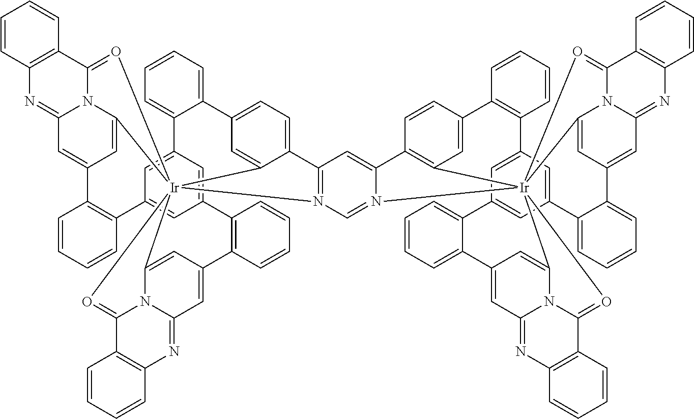

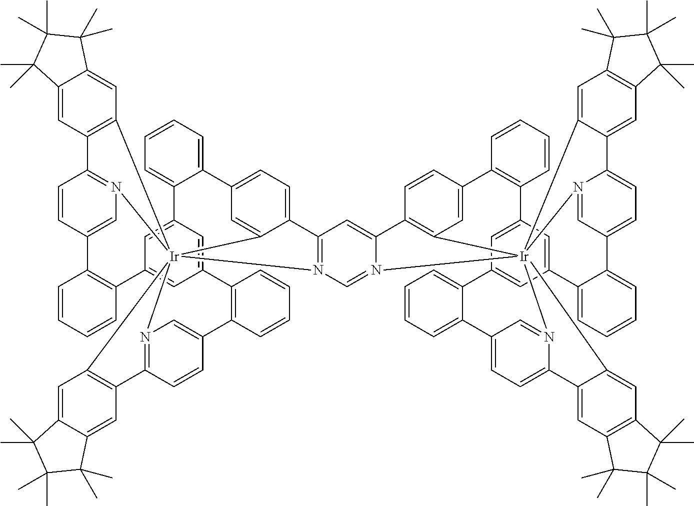

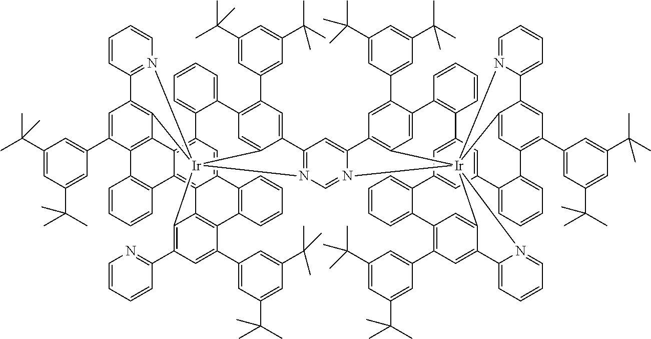

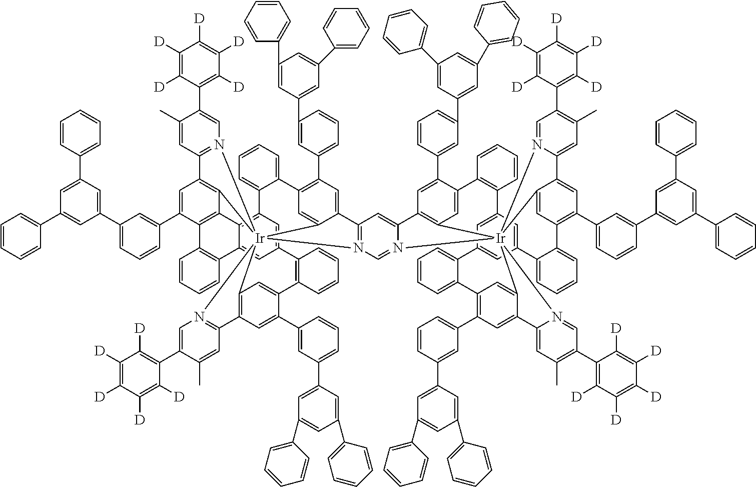

In the mixtures of the invention or in the light-emitting layer of the device of the invention, any mixture M1, M2, M3, M4, M5, M6, M7, M8, M9, M10, M11, M12, M13, M14, M15, M16, M17, M18, M19, M20, M21, M22, M23, M24, M25, M26, M27, M28, M29, M30, M31, M32, M33, M34, M35, M36, M37, M38, M39, M40, M41, M42, M43, M44, M45, M46, M47, M48, M49, M50, M51, M52, M53, M54, M55, M56, M57, M58, M59, M60, M61, M62, M63, M64, M65, M66, M67, M68, M69, M70, M71, M72, M73, M74, M75, M76, M77, M78, M79, M80, M81, M82, M83, M84, M85, M86, M87, M88, M89, M90, M91, M92, M93, M94, M95, M96, M97, M98, M99, M100, M101, M102, M103, M104, M105, M106, M107, M108, M109, M110, M111, M112, M113, M114, M115, M116, M117, M118, M119, M120, M121, M122, M123, M124, M125, M126, M127, M128, M129, M130, M131, M132, M133, M134, M135, M136, M137, M138, M139, M140, M141, M142, M143, M144, M145, M146, M147, M148, M149, M150, M151, M152, M153, M154, M155, M156, M157, M158, M159, M160, M161, M162, M163, M164, M165, M166, M167, M168, M169, M170, M171, M172, M173, M174, M175, M176, M177, M178, M179, M180, M181, M182, M183, M184, M185, M186, M187, M188, M189, M190, M191, M192, M193, M194, M195, M196, M197, M198, M199, M200, M201, M202, M203, M204, M205, M206, M207, M208, M209, M210, M211, M212, M213, M214, M215, M216, M217, M218, M219, M220, M221, M222, M223, M224, M225, M226, M227, M228, M229, M230, M231, M232, M233, M234, M235, M236, M237, M238, M239, M240, M241, M242, M243, M244, M245, M246, M247, M248, M249, M250, M251, M252, M253, M254, M255, M256, M257, M258, M259, M260, M261, M262, M263, M264, M265, M266, M267, M268, M269, M270, M271, M272, M273, M274, M275, M276, M277, M278, M279, M280, M281, M282, M283, M284, M285, M286, M287, M288, M289, M290, M291, M292, M293, M294, M295, M296, M297, M298, M299, M300, M301, M302, M303, M304, M305, M306, M307, M308, M309, M310, M311, M312, M313, M314, M315, M316, M317, M318, M319, M320, M321, M322, M323, M324, M325, M326, M327, M328, M329, M330, M331, M332, M333, M334, M335, M336, M337, M338, M339, M340, M341, M342, M343, M344, M345, M346, M347, M348, M349, M350, M351, M352, M353, M354, M355, M356, M357, M358, M359, M360, M361, M362, M363, M364, M365, M366, M367, M368, M369, M370, M371, M372, M373, M374, M375, M376, M377, M378, M379, M380, M381, M382, M383, M384, M385, M386, M387, M388, M389, M390, M391, M392, M393, M394, M395, M396, M397, M398, M399, M400, M401, M402, M403, M404, M405, M406, M407, M408, M409, M410, M411, M412, M413, M414, M415, M416, M417, M418, M419, M420, M421, M422, M423, M424, M425, M426, M427, M428, M429, M430, M431, M432, M433, M434, M435, M436, M437, M438, M439, M440, M441, M442, M443, M444, M445, M446, M447, M448, M449, M450, M451, M452, M453, M454, M455, M456, M457, M458, M459, M460, M461, M462, M463, M464, M465, M466, M467, M468, M469, M470, M471, M472, M473, M474, M475, M476, M477, M478, M479, M480, M481, M482, M483, M484, M485, M486, M487, M488, M489, M490, M491, M492, M493, M494, M495, M496, M497, M498, M499, M500, M501, M502, M503, M504, M505, M506, M507, M508, M509, M510, M511, M512, M513, M514, M515, M516, M517, M518, M519, M520, M521, M522, M523, M524, M525, M526, M527, M528, M529, M530, M531, M532, M533, M534, M535, M536, M537, M538, M539, M540, M541, M542, M543, M544, M545, M546, M547, M548, M549, M550, M551, M552, M553, M554, M555, M556, M557, M558, M559, M560, M561, M562, M563, M564, M565, M566, M567, M568, M569, M570, M571, M572, M573, M574, M575, M576, M577, M578, M579, M580, M581, M582, M583, M584, M585, M586, M587, M588, M589, M590, M591, M592, M593, M594, M595, M596, M597, M598, M599, M600, M601, M602, M603, M604, M605, M606, M607, M608, M609, M610, M611, M612, M613, M614, M615, M616, M617, M618, M619, M620, M621, M622, M623, M624, M625, M626, M627, M628, M629, M630, M631, M632, M633, M634, M635, M636, M637, M638, M639, M640, M641, M642, M643, M644, M645, M646, M647, M648, M649, M650, M651, M652, M653, M654, M655, M656, M657, M658, M659, M660, M661, M662, M663, M664, M665, M666, M667, M668, M669, M670, M671, M672, M673, M674, M675, M676, M677, M678, M679, M680, M681, M682, M683, M684, M685, M686, M687, M688, M689, M690, M691, M692, M693, M694, M695, M696, M697, M698, M699, M700, M701, M702, M703, M704, M705, M706, M707, M708, M709, M710, M711, M712, M713, M714, M715, M716, M717, M718, M719, M720, M721, M722, M723, M724, M725, M726, M727, M728, M729, M730, M731, M732, M733, M734, M735, M736, M737, M738, M739, M740, M741, M742, M743, M744, M745, M746, M747, M748, M749, M750, M751, M752, M753, M754, M755, M756, M757, M758, M759, M760, M761, M762, M763, M764, M765, M766, M767, M768, M769, M770, M771, M772, M773, M774, M775, M776, M777, M778, M779, M780, M781, M782, M783, M784, M785, M786, M787, M788, M789, M790, M791, M792, M793, M794, M795, M796, M797, M798, M799, M800, M801, M802, M803, M804, M805, M806, M807, M808, M809, M810, M811, M812, M813, M814, M815, M816, M817, M818, M819, M820, M821, M822, M823, M824, M825, M826, M827, M828, M829, M830, M831, M832, M833, M834, M835, M836, M837, M838, M839, M840, M841, M842, M843, M844, M845, M846, M847, M848, M849, M850, M851, M852, M853, M854, M855, M856, M857, M858, M859, M860, M861, M862, M863, M864, M865, M866, M867, M868, M869, M870, M871, M872, M873, M874, M875, M876, M877, M878, M879, M880, M881, M882, M883, M884, M885, M886, M887, M888, M889, M890, M891, M892, M893, M894, M895, M896, M897, M898, M899, M900, M901, M902, M903, M904, M905, M906, M907, M908, M909, M910, M911, M912, M913, M914, M915, M916, M917, M918, M919, M920, M921, M922, M923, M924, M925, M926, M927, M928, M929, M930, M931, M932, M933, M934, M935, M936, M937, M938, M939, M940, M941, M942, M943, M944, M945, M946, M947, M948, M949, M950, M951, M952, M953, M954, M955, M956, M957, M958, M959, M960, M961, M962, M963, M964, M965, M966, M967, M968, M969, M970, M971, M972, M973, M974, M975, M976, M977, M978, M979, M980, M981, M982, M983, M984, M985, M986, M987, M988, M989, M990, M991, M992, M993, M994, M995, M996, M997, M998, M999, M1000, M1001, M1002, M1003, M1004, M1005, M1006, M1007, M1008, M1009, M1010, M1011, M1012, M1013, M1014, M1015, M1016, M1017, M1018, M1019, M1020, M1021, M1022, M1023, M1024, M1025, M1026, M1027, M1028, M1029, M1030, M1031, M1032, M1033, M1034, M1035, M1036, M1037, M1038, M1039, M1040, M1041, M1042, M1043, M1044, M1045, M1046, M1047, M1048, M1049, M1050, M1051, M1052, M1053, M1054, M1055, M1056, M1057, M1058, M1059, M1060, M1061, M1062, M1063, M1064, M1065, M1066, M1067, M1068, M1069, M1070, M1071, M1072, M1073, M1074, M1075, M1076, M1077, M1078, M1079, M1080, M1081, M1082, M1083, M1084, M1085, M1086, M1087, M1088, M1089, M1090, M1091, M1092, M1093, M1094, M1095, M1096, M1097, M1098, M1099, M1100, M1101, M1102, M1103, M1104, M1105, M1106, M1107, M1108, M1109, M1110, M1111, M1112, M1113, M1114, M1115, M1116, M1117, M1118, M1119, M1120, M1121, M1122, M1123, M1124, M1125, M1126, M1127, M1128, M1129, M1130, M1131, M1132, M1133 or M1134, 1135, M1136, M1137, M1138, M1139, M1140, M1141, M1142, M1143, M1144, M1145, M1146, M1147, M1148, M1149, M1150, M1151, M1152, M1153, M1154, M1155, M1156, M1157, M1158, M1159, M1160, M1161, M1162, M1163, M1164, M1165, M1166, M1167, M1168, M1169, M1170, M1171, M1172, M1173, M1174, M1175, M1176, M1177, M1178, M1179, M1180, M1181, M1182, M1183, M1184, M1185, M1186, M1187, M1188, M1189, M1190, M1191, M1192, M1193, M1194, M1195, M1196, M1197, M1198, M1199, M1200, M1201, M1202, M1203, M1204, M1205, M1206, M1207, M1208, M1209, M1210, M1211, M1212, M1213, M1214, M1215, M1216, M1217, M1218, M1219, M1220, M1221, M1222, M1223, M1224, M1225, M1226, M1227, M1228, M1229, M1230, M1231, M1232, M1233, M1234, M1235, M1236, M1237, M1238, M1239, M1240, M1241, M1242, M1243, M1244, M1245, M1246, M1247, M1248, M1249, M1250, M1251, M1252, M1253, M1254, M1255, M1256, M1257, M1258, M1259, M1260, M1261, M1262, M1263, M1264, M1265, M1266, M1267, M1268, M1269, M1270, M1271, M1272, M1273, M1274, M1275, M1276, M1277, M1278, M1279, M1280, M1281, M1282, M1283, M1284, M1285, M1286, M1287, M1288, M1289, M1290, M1291, M1292, M1293, M1294, M1295, M1296, M1297, M1298, M1299, M1300, M1301, M1302, M1303, M1304, M1305, M1306, M1307, M1308, M1309, M1310, M1311, M1312, M1313, M1314, M1315, M1316, M1317, M1318, M1319, M1320, M1321, M1322, M1323, M1324, M1325, M1326, M1327, M1328, M1329, M1330, M1331, M1332, M1333, M1334, M1335, M1336, M1337, M1338, M1339, M1340, M1341, M1342, M1343, M1344 is preferably combined with a compound of the formula (IIIa) or a compound of the formulae (1) to (VI) or a compound from table 6.

The light-emitting layer in the organic electroluminescent device of the invention, comprising at least one phosphorescent emitter, is preferably an infrared-emitting or yellow-, orange-, red-, green-, blue- or ultraviolet-emitting layer, more preferably a yellow- or green-emitting layer and most preferably a green-emitting layer.

A yellow-emitting layer is understood here to mean a layer having a photoluminescence maximum within the range from 540 to 570 nm. An orange-emitting layer is understood to mean a layer having a photoluminescence maximum within the range from 570 to 600 nm. A red-emitting layer is understood to mean a layer having a photoluminescence maximum within the range from 600 to 750 nm. A green-emitting layer is understood to mean a layer having a photoluminescence maximum within the range from 490 to 540 nm. A blue-emitting layer is understood to mean a layer having a photoluminescence maximum within the range from 440 to 490 nm. The photoluminescence maximum of the layer is determined here by measuring the photoluminescence spectrum of the layer having a layer thickness of 50 nm at room temperature, said layer having the inventive combination of the host materials of the formulae (1) and (2) and the appropriate emitter.

The photoluminescence spectrum of the layer is recorded, for example, with a commercial photoluminescence spectrometer.

The photoluminescence spectrum of the emitter chosen is generally measured in oxygen-free solution, 10⁻⁵ molar, at room temperature, a suitable solvent being any in which the chosen emitter dissolves in the concentration mentioned. Particularly suitable solvents are typically toluene or 2-methyl-THF, but also dichloromethane. Measurement is effected with a commercial photoluminescence spectrometer. The triplet energy T1 in eV is determined from the photoluminescence spectra of the emitters. Firstly, the peak maximum Plmax. (in nm) of the photoluminescence spectrum is determined. The peak maximum Plmax. (in nm) is then converted to eV by: E(T1 in eV)=1240/E(T1 in nm)=1240/PLmax. (in nm).

Preferred phosphorescent emitters are accordingly infrared emitters, preferably of the formula (IIIa), of the formulae (1) to (VI) or from table 6, the triplet energy T₁ of which is preferably ˜1.9 eV to ˜1.0 eV.

Preferred phosphorescent emitters are accordingly red emitters, preferably of the formula (IIIa), of the formulae (1) to (VI) or from table 6, the triplet energy T₁ of which is preferably ˜2.1 eV to ˜1.9 eV.

Preferred phosphorescent emitters are accordingly yellow emitters, preferably of the formula (IIIa), of the formulae (1) to (VI) or from table 6, the triplet energy T₁ of which is preferably ˜2.3 eV to ˜2.1 eV.

Preferred phosphorescent emitters are accordingly green emitters, preferably of the formula (IIIa), of the formulae (1) to (VI) or from table 6, the triplet energy T₁ of which is preferably ˜2.5 eV to ˜2.3 eV.

Preferred phosphorescent emitters are accordingly blue emitters, preferably of the formula (IIIa), of the formulae (1) to (VI) or from table 6, the triplet energy T₁ of which is preferably ˜3.1 eV to ˜2.5 eV.

Preferred phosphorescent emitters are accordingly ultraviolet emitters of the formula (IIIa), of the formulae (1) to (VI) or from table 6, the triplet energy T₁ of which is preferably ˜4.0 eV to ˜3.1 eV.

Particularly preferred phosphorescent emitters are accordingly green or yellow emitters, preferably of the formula (IIIa), of the formulae (1) to (VI) or from table 6, as described above.

Very particularly preferred phosphorescent emitters are accordingly green emitters, preferably of the formula (IIIa), of the formulae (1) to (VI) or from table 6, the triplet energy T₁ of which is preferably ˜2.5 eV to ˜2.3 eV.

Most preferably, green emitters, preferably of the formula (IIIa), of the formulae (1) to (VI) or from table 6, as described above, are selected for the composition of the invention or emitting layer of the invention.

It is also possible for fluorescent emitters to be present in the light-emitting layer of the device of the invention.

Preferred fluorescent emitters are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9, 10 position. Aromatic pyreneamines, pyrenediamines, chryseneamines and chrysenediamines are defined analogously, where the diarylamino groups are bonded to the pyrene preferably in the 1 position or 1, 6 position. Further preferred fluorescent emitters are indenofluoreneamines or -diamines, for example according to WO 2006/108497 or WO 2006/122630, benzoindenofluoreneamines or -diamines, for example according to WO 2008/006449, and dibenzoindenofluoreneamines or -diamines, for example according to WO 2007/140847, and the indenofluorene derivatives having fused aryl groups disclosed in WO 2010/012328.

In a further preferred embodiment of the invention, the at least one light-emitting layer of the organic electroluminescent device, as well as the host materials 1 and 2, as described above or described as preferred, may comprise further host materials or matrix materials, called mixed matrix systems. The mixed matrix systems preferably comprise three or four different matrix materials, more preferably three different matrix materials (in other words, one further matrix component in addition to the host materials 1 and 2, as described above). Particularly suitable matrix materials which can be used in combination as matrix component in a mixed matrix system are selected from wide-band gap materials, bipolar host materials, electron transport materials (ETM) and hole transport materials (HTM).

A wide-band gap material is understood herein to mean a material within the scope of the disclosure of U.S. Pat. No. 7,294,849 which is characterized by a band gap of at least 3.5 eV, the band gap being understood to mean the gap between the HOMO and LUMO energy of a material.

In one embodiment of the present invention, the mixture does not comprise any further constituents, i.e. functional materials, aside from the constituents of electron-transporting host material of the formula (1) and hole-transporting host material of the formula (2). These are material mixtures that are used as such for production of the light-emitting layer. These mixtures are also referred to as premix systems that are used as the sole material source in the vapour deposition of the host materials for the light-emitting layer and have a constant mixing ratio in the vapour deposition. In this way, it is possible in a simple and rapid manner to achieve the vapour deposition of a layer with homogeneous distribution of the components without the need for precise actuation of a multitude of material sources.

In an alternative embodiment of the present invention, the mixture also comprises the phosphorescent emitter, as described above, in addition to the constituents of electron-transporting host material of the formula (1) and hole-transporting host material of the formula (2). In the case of a suitable mixing ratio in the vapour deposition, this mixture may also be used as the sole material source, as described above.

The components or constituents of the light-emitting layer of the device of the invention may thus be processed by vapour deposition or from solution. The material combination of host materials 1 and 2, as described above or described as preferred, optionally with the phosphorescent emitter, as described above or described as preferred, is provided for the purpose in a formulation containing at least one solvent. These formulations may, for example, be solutions, dispersions or emulsions. For this purpose, it may be preferable to use mixtures of two or more solvents.

The present invention therefore further provides a formulation comprising an inventive mixture of host materials 1 and 2, as described above, optionally in combination with a phosphorescent emitter, as described above or described as preferred, and at least one solvent.

Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, hexamethylindane or mixtures of these solvents.

The formulation here may also comprise at least one further organic or inorganic compound which is likewise used in the light-emitting layer of the device of the invention, especially a further emitting compound and/or a further matrix material.

The light-emitting layer in the device of the invention, according to the preferred embodiments and the emitting compound, contains preferably between 99.9% and 1% by volume, further preferably between 99% and 10% by volume, especially preferably between 98% and 60% by volume, very especially preferably between 97% and 80% by volume, of matrix material composed of at least one compound of the formula (1) and at least one compound of the formula (2) according to the preferred embodiments, based on the overall composition of emitter and matrix material. Correspondingly, the light-emitting layer in the device of the invention preferably contains between 0.1% and 99% by volume, further preferably between 1% and 90% by volume, more preferably between 2% and 40% by volume, most preferably between 3% and 20% by volume, of the emitter based on the overall composition of the light-emitting layer composed of emitter and matrix material. If the compounds are processed from solution, preference is given to using the corresponding amounts in % by weight rather than the above-specified amounts in % by volume.

The light-emitting layer in the device of the invention, according to the preferred embodiments and the emitting compound, preferably contains the matrix material of the formula (1) and the matrix material of the formula (2) in a percentage by volume ratio between 3:1 and 1:3, preferably between 1:2.5 and 1:1, more preferably between 1:2 and 1:1. If the compounds are processed from solution, preference is given to using the corresponding ratio in % by weight rather than the above-specified ratio in % by volume.

The present invention also relates to an organic electroluminescent device as described above or described as preferred, wherein the organic layer comprises a hole injection layer (HIL) and/or a hole transport layer (HTL), the hole-injecting material and hole-transporting material of which is a monoamine that does not contain a carbazole unit. The hole-injecting material and hole-transporting material preferably comprises a monoamine containing a fluorenyl or bispirofluorenyl group, but no carbazole unit.

Preferred monoamines which are used in accordance with the invention in the organic layer of the device of the invention may be described by the formula (IVa)

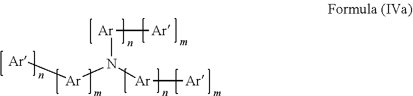

where the symbols and indices for this formula (IVa) are defined as follows:

Ar and Ar′ at each instance are independently an aromatic ring system having 6 to 40 ring atoms or a heteroaromatic ring system having 7 to 40 ring atoms, with exclusion of carbazole units in the heteroaromatic ring system;

n at each instance is independently 0 or 1;

m at each instance is independently 0 or 1.

Preferably at least one Ar′ in formula (IVa) is a group of the following formulae (Va) or (Vb):

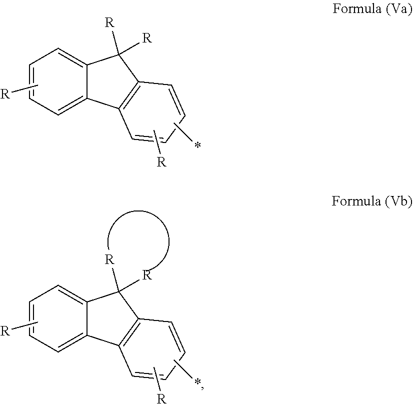

where R in formulae (Va) and (Vb) is the same or different at each instance and is selected from H, D, F, CN, a straight-chain alkyl group having 1 to 20 carbon atoms or a branched or cyclic alkyl group having 3 to 20 carbon atoms, where one or more nonadjacent CH₂ groups may be replaced by R²C═CR², O or S and where one or more hydrogen atoms may be replaced by D, F, or CN and where two R may form a cyclic or polycyclic ring and * denotes the attachment to the remainder of the formula (IVa).

Preferred monoamines which are used in accordance with the invention in the organic layer of the device of the invention are described in table 7.

TABLE 7

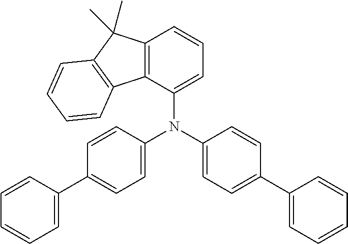 [SMILES: CC1(C)c2ccccc2-c2c(N(c3ccc(-c4ccccc4)cc3)c3ccc(-c4ccccc4)cc3)cccc21]

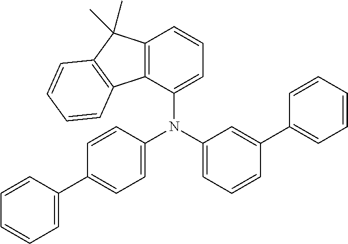 [SMILES: CC1(C)c2ccccc2-c2c(N(c3ccc(-c4ccccc4)cc3)c3cccc(-c4ccccc4)c3)cccc21]

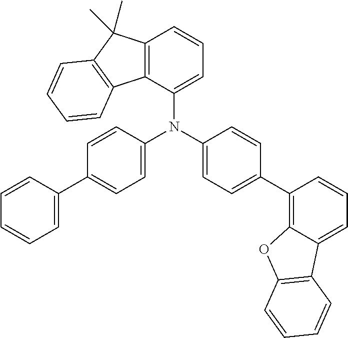 [SMILES: CC1(C)c2ccccc2-c2c(N(c3ccc(-c4ccccc4)cc3)c3ccc(-c4cccc5c4oc4ccccc45)cc3)cccc21]

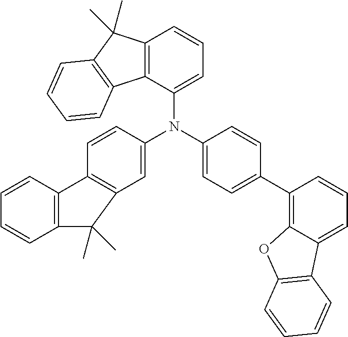 [SMILES: CC1(C)c2ccccc2-c2ccc(N(c3ccc(-c4cccc5c4oc4ccccc45)cc3)c3cccc4c3-c3ccccc3C4(C)C)cc21]

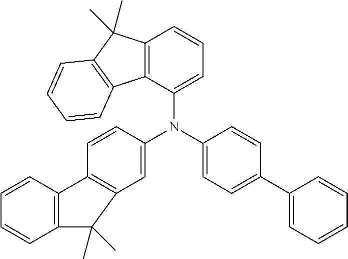 [SMILES: CC1(C)c2ccccc2-c2ccc(N(c3ccc(-c4ccccc4)cc3)c3cccc4c3-c3ccccc3C4(C)C)cc21]

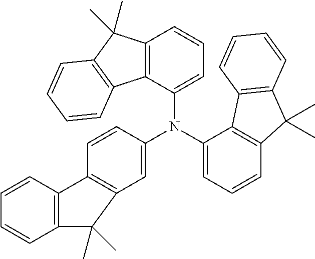 [SMILES: CC1(C)c2ccccc2-c2ccc(N(c3cccc4c3-c3ccccc3C4(C)C)c3cccc4c3-c3ccccc3C4(C)C)cc21]

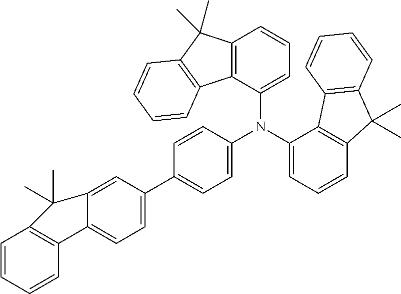 [SMILES: CC1(C)c2ccccc2-c2ccc(-c3ccc(N(c4cccc5c4-c4ccccc4C5(C)C)c4cccc5c4-c4ccccc4C5(C)C)cc3)cc21]

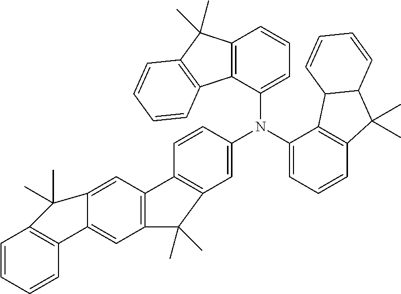 [SMILES: CC1(C)c2ccccc2-c2cc3c(cc21)-c1ccc(N(c2cccc4c2-c2ccccc2C4(C)C)c2cccc4c2C2C=CC=CC2C4(C)C)cc1C3(C)C]

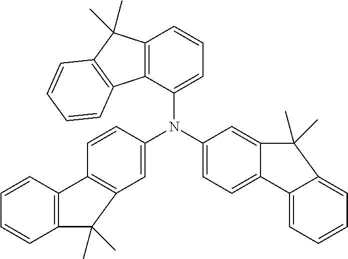 [SMILES: CC1(C)c2ccccc2-c2ccc(N(c3ccc4c(c3)C(C)(C)c3ccccc3-4)c3cccc4c3-c3ccccc3C4(C)C)cc21]

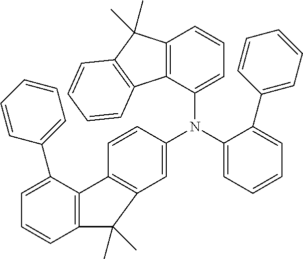 [SMILES: CC1(C)c2cc(N(c3ccccc3-c3ccccc3)c3cccc4c3-c3ccccc3C4(C)C)ccc2-c2c(-c3ccccc3)cccc21]

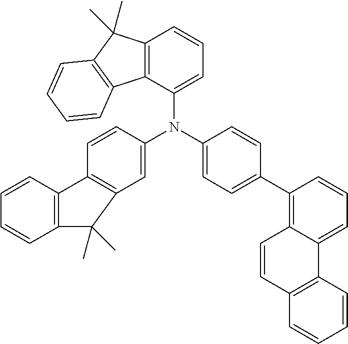 [SMILES: CC1(C)c2ccccc2-c2ccc(N(c3ccc(-c4cccc5c4ccc4ccccc45)cc3)c3cccc4c3-c3ccccc3C4(C)C)cc21]

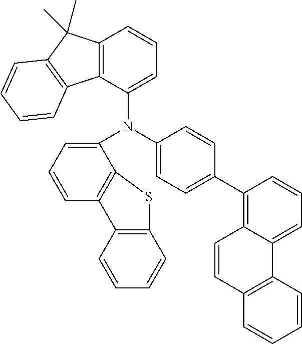 [SMILES: CC1(C)c2ccccc2-c2c(N(c3ccc(-c4cccc5c4ccc4ccccc45)cc3)c3cccc4c3sc3ccccc34)cccc21]

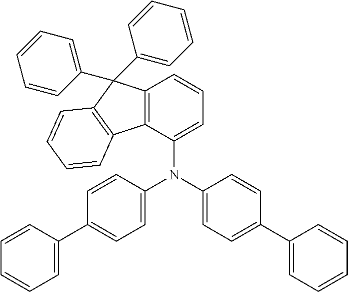 [SMILES: c1ccc(-c2ccc(N(c3ccc(-c4ccccc4)cc3)c3cccc4c3-c3ccccc3C4(c3ccccc3)c3ccccc3)cc2)cc1]

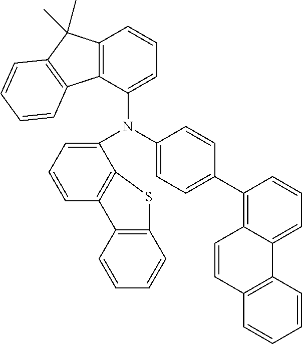 [SMILES: CC1(C)c2ccccc2-c2c(N(c3ccc(-c4cccc5c4ccc4ccccc45)cc3)c3cccc4c3sc3ccccc34)cccc21]

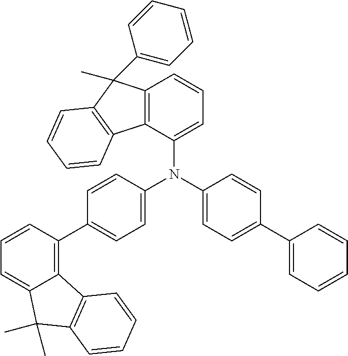 [SMILES: CC1(C)c2ccccc2-c2c(-c3ccc(N(c4ccc(-c5ccccc5)cc4)c4cccc5c4-c4ccccc4C5(C)c4ccccc4)cc3)cccc21]

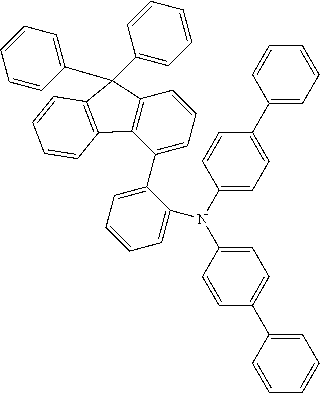 [SMILES: c1ccc(-c2ccc(N(c3ccc(-c4ccccc4)cc3)c3ccccc3-c3cccc4c3-c3ccccc3C4(c3ccccc3)c3ccccc3)cc2)cc1]

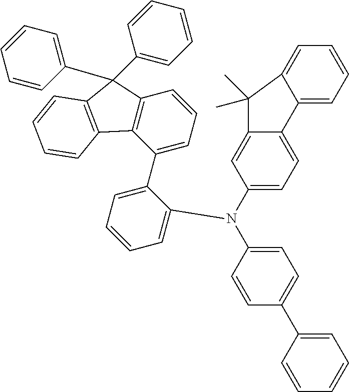 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21]

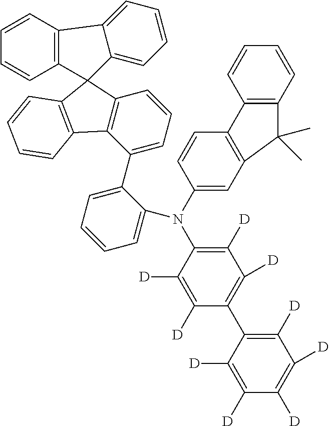 [SMILES: [2H]c1c([2H])c([2H])c(-c2c([2H])c([2H])c(N(c3ccc4c(c3)C(C)(C)c3ccccc3-4)c3ccccc3-c3cccc4c3-c3ccccc3C43c4ccccc4-c4ccccc43)c([2H])c2[2H])c([2H])c1[2H]]

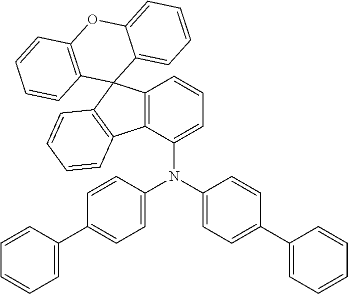 [SMILES: c1ccc(-c2ccc(N(c3ccc(-c4ccccc4)cc3)c3cccc4c3-c3ccccc3C43c4ccccc4Oc4ccccc43)cc2)cc1]

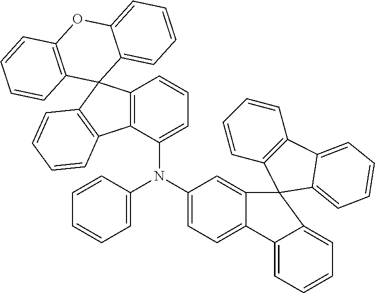 [SMILES: c1ccc(N(c2ccc3c(c2)C2(c4ccccc4-c4ccccc42)c2ccccc2-3)c2cccc3c2-c2ccccc2C32c3ccccc3Oc3ccccc32)cc1]

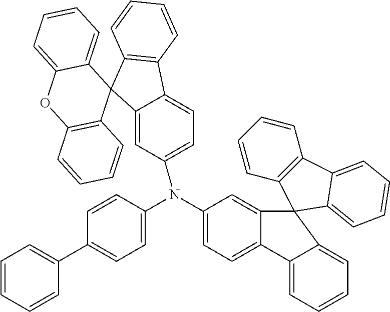 [SMILES: c1ccc(-c2ccc(N(c3ccc4c(c3)C3(c5ccccc5Oc5ccccc53)c3ccccc3-4)c3ccc4c(c3)C3(c5ccccc5-c5ccccc53)c3ccccc3-4)cc2)cc1]

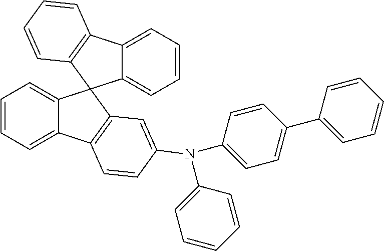 [SMILES: c1ccc(-c2ccc(N(c3ccccc3)c3ccc4c(c3)C3(c5ccccc5-c5ccccc53)c3ccccc3-4)cc2)cc1]

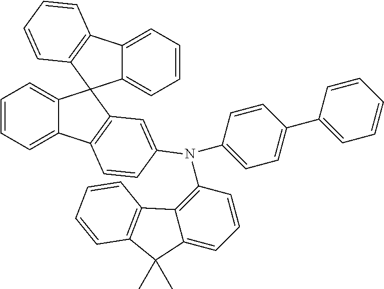 [SMILES: CC1(C)c2ccccc2-c2c(N(c3ccc(-c4ccccc4)cc3)c3ccc4c(c3)C3(c5ccccc5-c5ccccc53)c3ccccc3-4)cccc21]

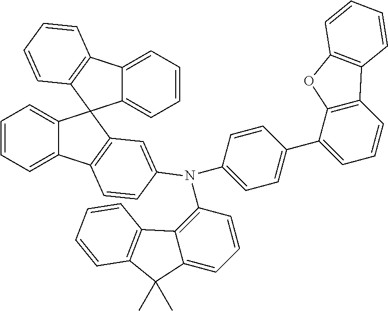 [SMILES: CC1(C)c2ccccc2-c2c(N(c3ccc(-c4cccc5c4oc4ccccc45)cc3)c3ccc4c(c3)C3(c5ccccc5-c5ccccc53)c3ccccc3-4)cccc21]

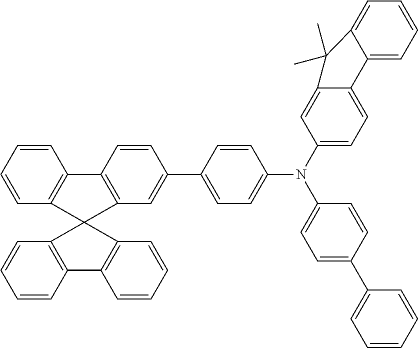 [SMILES: CC1(C)c2ccccc2-c2ccc(N(c3ccc(-c4ccccc4)cc3)c3ccc(-c4ccc5c(c4)C4(c6ccccc6-c6ccccc64)c4ccccc4-5)cc3)cc21]

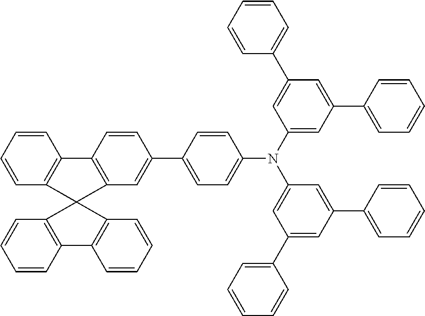 [SMILES: c1ccc(-c2cc(-c3ccccc3)cc(N(c3ccc(-c4ccc5c(c4)C4(c6ccccc6-c6ccccc64)c4ccccc4-5)cc3)c3cc(-c4ccccc4)cc(-c4ccccc4)c3)c2)cc1]

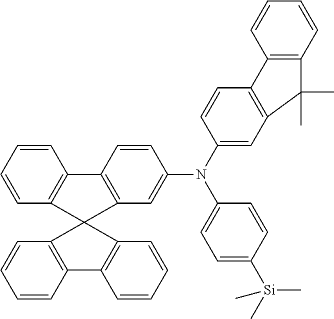 [SMILES: CC1(C)c2ccccc2-c2ccc(N(c3ccc([Si](C)(C)C)cc3)c3ccc4c(c3)C3(c5ccccc5-c5ccccc53)c3ccccc3-4)cc21]

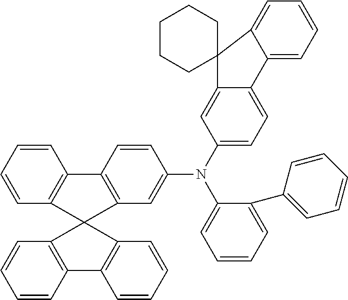 [SMILES: c1ccc(-c2ccccc2N(c2ccc3c(c2)C2(CCCCC2)c2ccccc2-3)c2ccc3c(c2)C2(c4ccccc4-c4ccccc42)c2ccccc2-3)cc1]

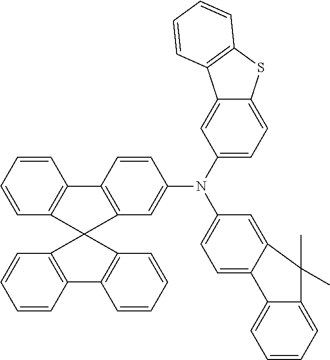 [SMILES: CC1(C)c2ccccc2-c2ccc(N(c3ccc4c(c3)C3(c5ccccc5-c5ccccc53)c3ccccc3-4)c3ccc4sc5ccccc5c4c3)cc21]

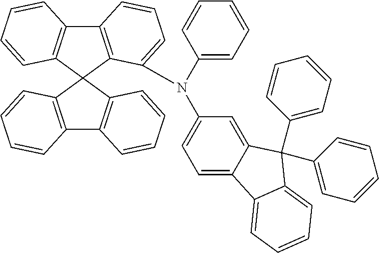 [SMILES: c1ccc(N(c2ccc3c(c2)C(c2ccccc2)(c2ccccc2)c2ccccc2-3)c2cccc3c2C2(c4ccccc4-c4ccccc42)c2ccccc2-3)cc1]

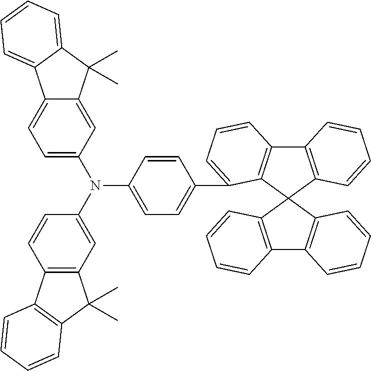 [SMILES: CC1(C)c2ccccc2-c2ccc(N(c3ccc(-c4cccc5c4C4(c6ccccc6-c6ccccc64)c4ccccc4-5)cc3)c3ccc4c(c3)C(C)(C)c3ccccc3-4)cc21]

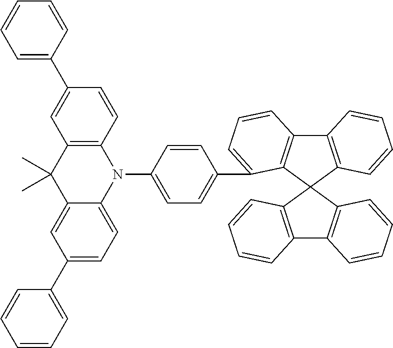 [SMILES: CC1(C)c2cc(-c3ccccc3)ccc2N(c2ccc(-c3cccc4c3C3(c5ccccc5-c5ccccc53)c3ccccc3-4)cc2)c2ccc(-c3ccccc3)cc21]

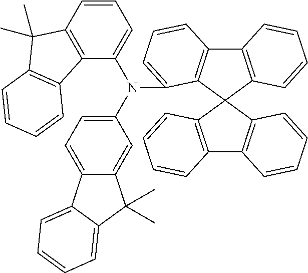 [SMILES: CC1(C)c2ccccc2-c2ccc(N(c3cccc4c3-c3ccccc3C4(C)C)c3cccc4c3C3(c5ccccc5-c5ccccc53)c3ccccc3-4)cc21]

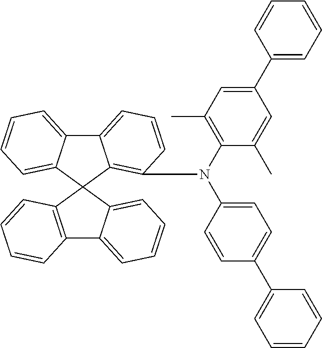 [SMILES: Cc1cc(-c2ccccc2)cc(C)c1N(c1ccc(-c2ccccc2)cc1)c1cccc2c1C1(c3ccccc3-c3ccccc31)c1ccccc1-2]

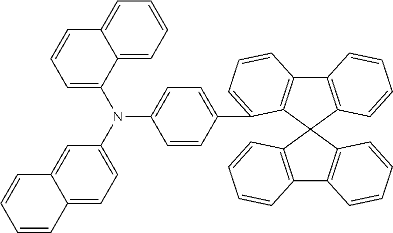 [SMILES: c1ccc2c(c1)-c1ccccc1C21c2ccccc2-c2cccc(-c3ccc(N(c4ccc5ccccc5c4)c4cccc5ccccc45)cc3)c21]

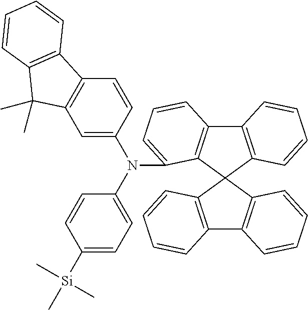 [SMILES: CC1(C)c2ccccc2-c2ccc(N(c3ccc([Si](C)(C)C)cc3)c3cccc4c3C3(c5ccccc5-c5ccccc53)c3ccccc3-4)cc21]

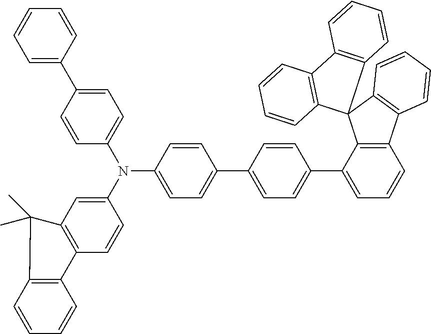 [SMILES: CC1(C)c2ccccc2-c2ccc(N(c3ccc(-c4ccccc4)cc3)c3ccc(-c4ccc(-c5cccc6c5C5(c7ccccc7-c7ccccc75)c5ccccc5-6)cc4)cc3)cc21]

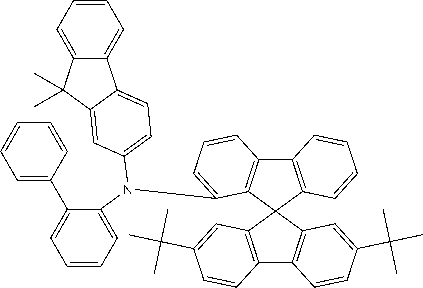 [SMILES: CC(C)(C)c1ccc2c(c1)C1(c3cc(C(C)(C)C)ccc3-2)c2ccccc2-c2cccc(N(c3ccc4c(c3)C(C)(C)c3ccccc3-4)c3ccccc3-c3ccccc3)c21]

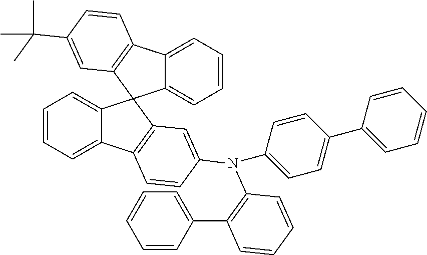 [SMILES: CC(C)(C)c1ccc2c(c1)C1(c3ccccc3-c3ccc(N(c4ccc(-c5ccccc5)cc4)c4ccccc4-c4ccccc4)cc31)c1ccccc1-2]

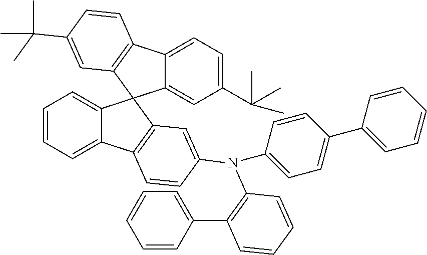 [SMILES: CC(C)(C)c1ccc2c(c1)C1(c3ccccc3-c3ccc(N(c4ccc(-c5ccccc5)cc4)c4ccccc4-c4ccccc4)cc31)c1cc(C(C)(C)C)ccc1-2]

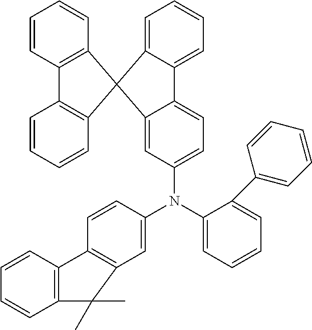 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

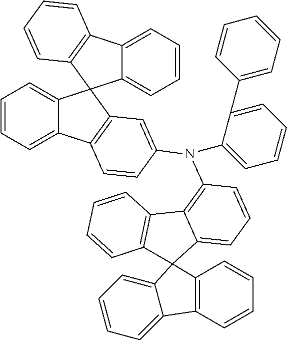 [SMILES: c1ccc(-c2ccccc2N(c2ccc3c(c2)C2(c4ccccc4-c4ccccc42)c2ccccc2-3)c2cccc3c2-c2ccccc2C32c3ccccc3-c3ccccc32)cc1]

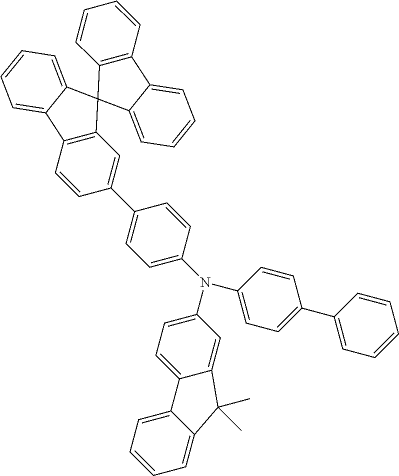 [SMILES: CC1(C)c2ccccc2-c2ccc(N(c3ccc(-c4ccccc4)cc3)c3ccc(-c4ccc5c(c4)C4(c6ccccc6-c6ccccc64)c4ccccc4-5)cc3)cc21]

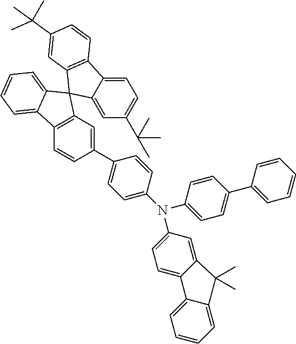 [SMILES: CC(C)(C)c1ccc2c(c1)C1(c3ccccc3-c3ccc(-c4ccc(N(c5ccc(-c6ccccc6)cc5)c5ccc6c(c5)C(C)(C)c5ccccc5-6)cc4)cc31)c1cc(C(C)(C)C)ccc1-2]

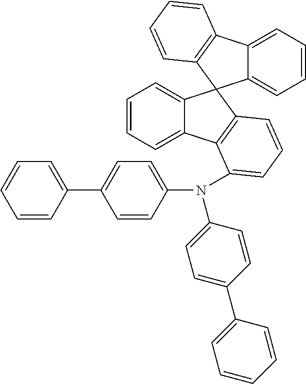 [SMILES: c1ccc(-c2ccc(N(c3ccc(-c4ccccc4)cc3)c3cccc4c3-c3ccccc3C43c4ccccc4-c4ccccc43)cc2)cc1]

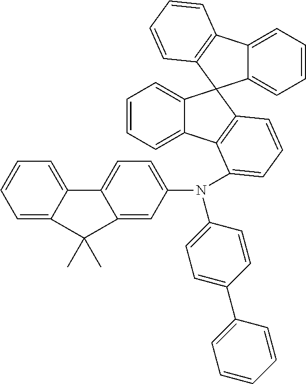 [SMILES: CC1(C)c2ccccc2-c2ccc(N(c3ccc(-c4ccccc4)cc3)c3cccc4c3-c3ccccc3C43c4ccccc4-c4ccccc43)cc21]

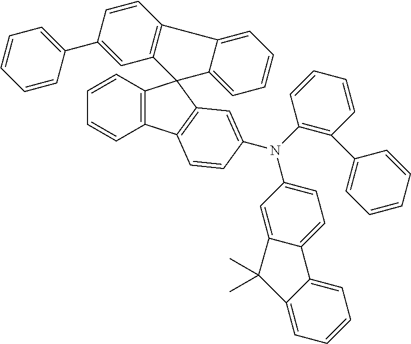 [SMILES: CC1(C)c2ccccc2-c2ccc(N(c3ccc4c(c3)C3(c5ccccc5-c5ccc(-c6ccccc6)cc53)c3ccccc3-4)c3ccccc3-c3ccccc3)cc21]

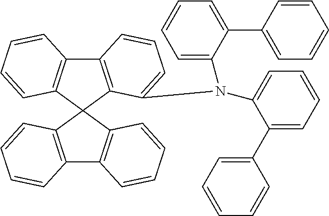 [SMILES: c1ccc(-c2ccccc2N(c2ccccc2-c2ccccc2)c2cccc3c2C2(c4ccccc4-c4ccccc42)c2ccccc2-3)cc1]

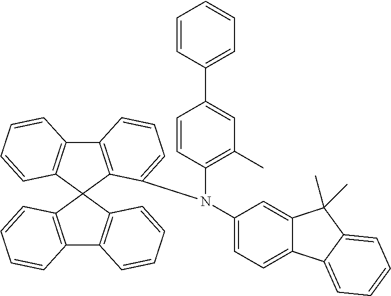 [SMILES: Cc1cc(-c2ccccc2)ccc1N(c1ccc2c(c1)C(C)(C)c1ccccc1-2)c1cccc2c1C1(c3ccccc3-c3ccccc31)c1ccccc1-2]

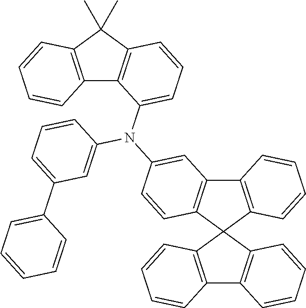 [SMILES: CC1(C)c2ccccc2-c2c(N(c3cccc(-c4ccccc4)c3)c3ccc4c(c3)-c3ccccc3C43c4ccccc4-c4ccccc43)cccc21]

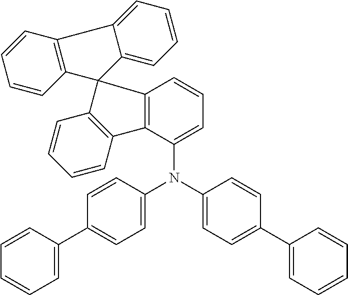 [SMILES: c1ccc(-c2ccc(N(c3ccc(-c4ccccc4)cc3)c3cccc4c3-c3ccccc3C43c4ccccc4-c4ccccc43)cc2)cc1]

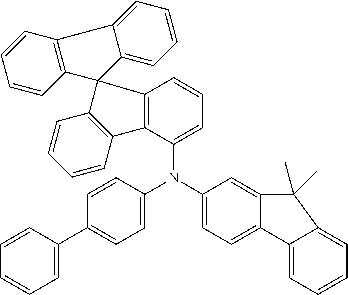 [SMILES: CC1(C)c2ccccc2-c2ccc(N(c3ccc(-c4ccccc4)cc3)c3cccc4c3-c3ccccc3C43c4ccccc4-c4ccccc43)cc21]

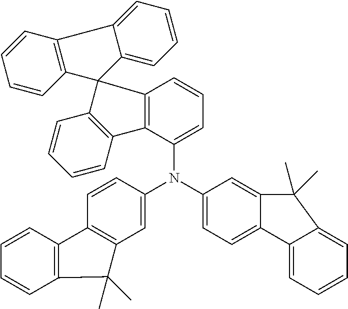 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21]

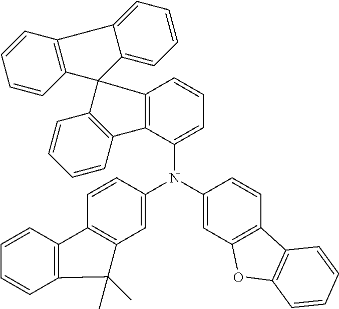 [SMILES: CC1(C)c2ccccc2-c2ccc(N(c3ccc4c(c3)oc3ccccc34)c3cccc4c3-c3ccccc3C43c4ccccc4-c4ccccc43)cc21]

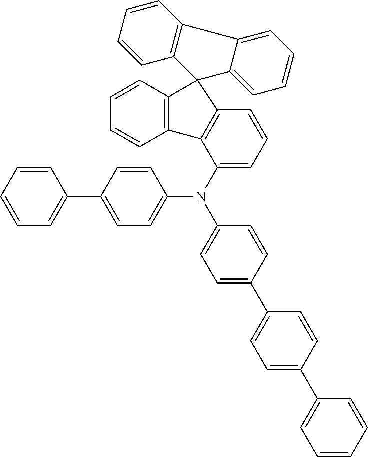 [SMILES: c1ccc(-c2ccc(-c3ccc(N(c4ccc(-c5ccccc5)cc4)c4cccc5c4-c4ccccc4C54c5ccccc5-c5ccccc54)cc3)cc2)cc1]

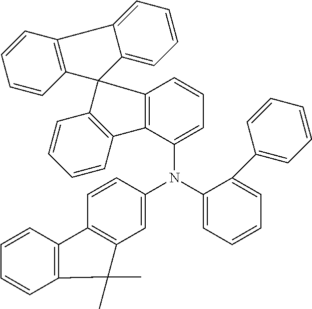 [SMILES: CC1(C)c2ccccc2-c2ccc(N(c3ccccc3-c3ccccc3)c3cccc4c3-c3ccccc3C43c4ccccc4-c4ccccc43)cc21]

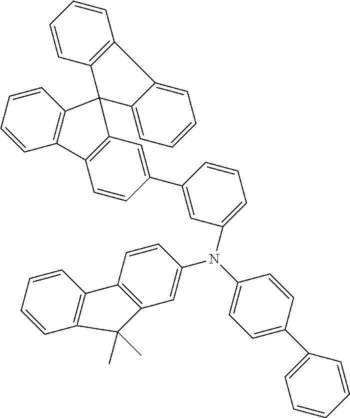 [SMILES: CC1(C)c2ccccc2-c2ccc(N(c3ccc(-c4ccccc4)cc3)c3cccc(-c4ccc5c(c4)C4(c6ccccc6-c6ccccc64)c4ccccc4-5)c3)cc21]

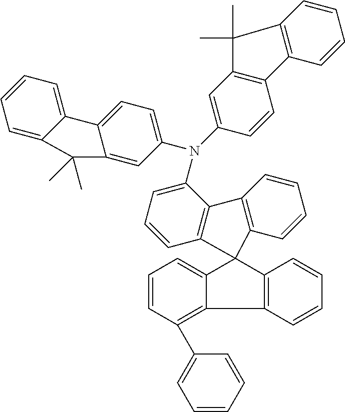 [SMILES: CC1(C)c2ccccc2-c2ccc(N(c3ccc4c(c3)C(C)(C)c3ccccc3-4)c3cccc4c3-c3ccccc3C43c4ccccc4-c4c(-c5ccccc5)cccc43)cc21]

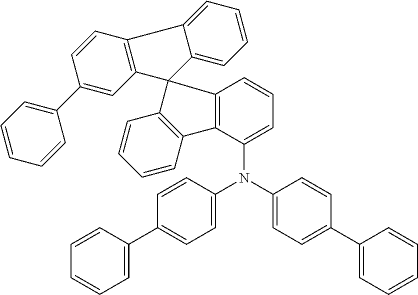 [SMILES: c1ccc(-c2ccc(N(c3ccc(-c4ccccc4)cc3)c3cccc4c3-c3ccccc3C43c4ccccc4-c4ccc(-c5ccccc5)cc43)cc2)cc1]

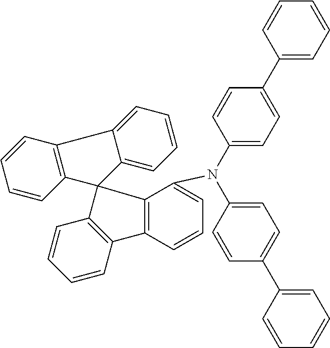 [SMILES: c1ccc(-c2ccc(N(c3ccc(-c4ccccc4)cc3)c3cccc4c3C3(c5ccccc5-c5ccccc53)c3ccccc3-4)cc2)cc1]

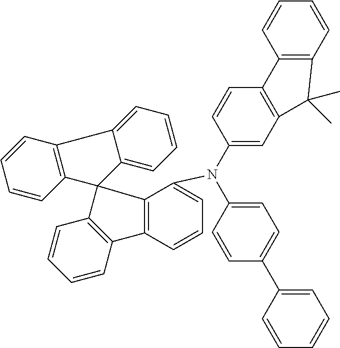 [SMILES: CC1(C)c2ccccc2-c2ccc(N(c3ccc(-c4ccccc4)cc3)c3cccc4c3C3(c5ccccc5-c5ccccc53)c3ccccc3-4)cc21]

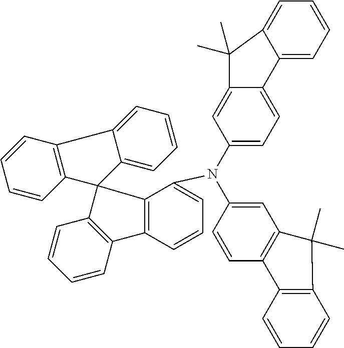 [SMILES: CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

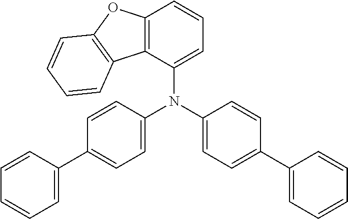 [SMILES: c1ccc(-c2ccc(N(c3ccc(-c4ccccc4)cc3)c3cccc4oc5ccccc5c34)cc2)cc1]

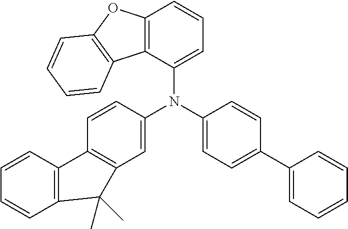 [SMILES: CC1(C)c2ccccc2-c2ccc(N(c3ccc(-c4ccccc4)cc3)c3cccc4oc5ccccc5c34)cc21]

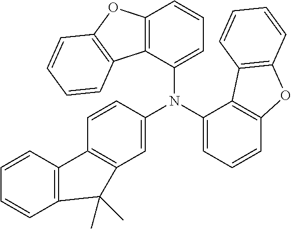 [SMILES: CC1(C)c2ccccc2-c2ccc(N(c3cccc4oc5ccccc5c34)c3cccc4oc5ccccc5c34)cc21]

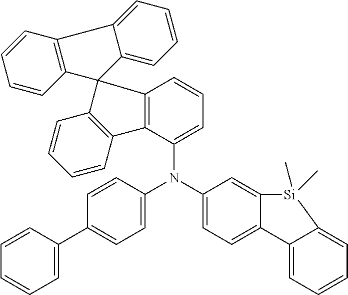 [SMILES: C[Si]1(C)c2ccccc2-c2ccc(N(c3ccc(-c4ccccc4)cc3)c3cccc4c3-c3ccccc3C43c4ccccc4-c4ccccc43)cc21]

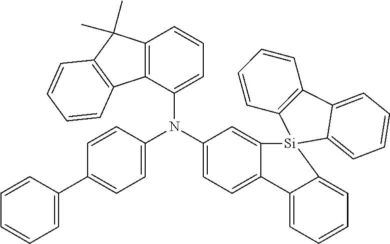 [SMILES: CC1(C)c2ccccc2-c2c(N(c3ccc(-c4ccccc4)cc3)c3ccc4c(c3)[Si]3(c5ccccc5-c5ccccc53)c3ccccc3-4)cccc21]

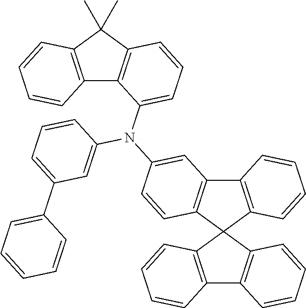 [SMILES: CC1(C)c2ccccc2-c2c(N(c3cccc(-c4ccccc4)c3)c3ccc4c(c3)-c3ccccc3C43c4ccccc4-c4ccccc43)cccc21]

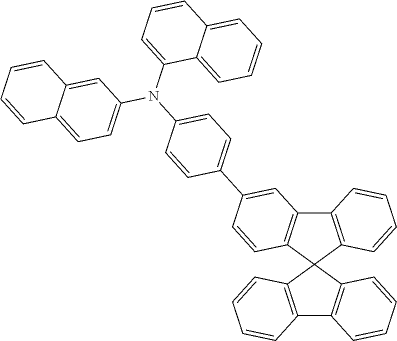 [SMILES: c1ccc2c(c1)-c1ccccc1C21c2ccccc2-c2cc(-c3ccc(N(c4ccc5ccccc5c4)c4cccc5ccccc45)cc3)ccc21]

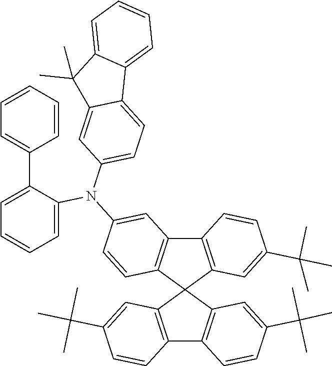 [SMILES: CC(C)(C)c1ccc2c(c1)C1(c3ccc(N(c4ccc5c(c4)C(C)(C)c4ccccc4-5)c4ccccc4-c4ccccc4)cc3-2)c2cc(C(C)(C)C)ccc2-c2ccc(C(C)(C)C)cc21]

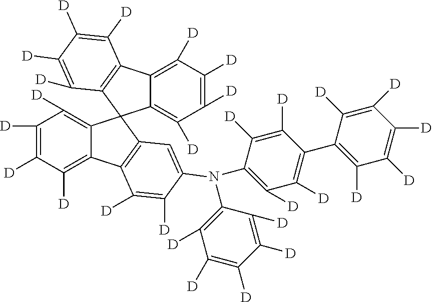 [SMILES: [2H]c1c(N(c2c([2H])c([2H])c([2H])c([2H])c2[2H])c2c([2H])c([2H])c(-c3c([2H])c([2H])c([2H])c([2H])c3[2H])c([2H])c2[2H])cc2c(c1[2H])-c1c([2H])c([2H])c([2H])c([2H])c1C21c2c([2H])c([2H])c([2H])c([2H])c2-c2c([2H])c([2H])c([2H])c([2H])c21]

Preferred hole transport materials are also, in combination with the compounds of the formula (IVa) or from table 7 or as alternatives to compounds of the formula (IVa) or from table 7, materials that can be used in a hole transport, hole injection or electron blocker layer, such as indenofluoreneamine derivatives (for example according to WO 06/122630 or WO 06/100896), the amine derivatives disclosed in EP 1661888, hexaazatriphenylene derivatives (for example according to WO 01/049806), amine derivatives with fused aromatic systems (for example according to U.S. Pat. No. 5,061,569), the amine derivatives disclosed in WO 95/09147, monobenzoindenofluoreneamines (for example according to WO 08/006449), dibenzoindenofluoreneamines (for example according to WO 07/140847), dihydroacridine derivatives (e.g. WO 2012/150001).

The sequence of layers in the organic electroluminescent device of the invention is preferably as follows: anode/hole injection layer/hole transport layer/emitting layer/electron transport layer/electron injection layer/cathode.

This sequence of the layers is a preferred sequence.

At the same time, it should be pointed out again that not all the layers mentioned need be present and/or that further layers may additionally be present.

The organic electroluminescent device of the invention may contain two or more emitting layers. At least one of the emitting layers is the light-emitting layer of the invention containing at least one compound of the formula (1) as host material 1 and at least one compound of the formula (2) as host material 2 as described above. More preferably, these emission layers in this case have several emission maxima between 380 nm and 750 nm overall, such that the overall result is white emission; in other words, various emitting compounds which may fluoresce or phosphoresce and which emit blue or yellow or orange or red light are used in the emitting layers. Especially preferred are three-layer systems, i.e. systems having three emitting layers, where the three layers show blue, green and orange or red emission (for the basic construction see, for example, WO 2005/011013). It should be noted that, for the production of white light, rather than a plurality of colour-emitting emitter compounds, an emitter compound used individually which emits over a broad wavelength range may also be suitable.

Suitable charge transport materials as usable in the hole injection or hole transport layer or electron blocker layer or in the electron transport layer of the organic electroluminescent device of the invention are, for example, the compounds disclosed in Y. Shirota et al., Chem. Rev. 2007, 107(4), 953-1010, or other materials as used in these layers according to the prior art.

Materials used for the electron transport layer may be any materials as used according to the prior art as electron transport materials in the electron transport layer. Especially suitable are aluminium complexes, for example Alq3, zirconium complexes, for example Zrq4, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Further suitable materials are derivatives of the abovementioned compounds as disclosed in JP 2000/053957, WO 2003/060956, WO 2004/028217, WO 2004/080975 and WO 2010/072300.

Suitable cathodes of the device of the invention are metals having a low work function, metal alloys or multilayer structures composed of various metals, for example alkaline earth metals, alkali metals, main group metals or lanthanoids (e.g. Ca, Ba, Mg, Al, In, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOx, Al/PtOx) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The organic electroluminescent device of the invention, in the course of production, is appropriately (according to the application) structured, contact-connected and finally sealed, since the lifetime of the devices of the invention is shortened in the presence of water and/or air.

The production of the device of the invention is not restricted here. It is possible that one or more organic layers, including the light-emitting layer, are coated by a sublimation method. In this case, the materials are applied by vapour deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

The organic electroluminescent device of the invention is preferably characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured (for example, M. S. Arnold et al., Appl. Phys. Lett. 2008, 92, 053301).

The organic electroluminescent device of the invention is further preferably characterized in that one or more organic layers comprising the composition of the invention are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble host materials 1 and 2 and phosphorescent emitters are needed. Processing from solution has the advantage that, for example, the light-emitting layer can be applied in a very simple and inexpensive manner. This technique is especially suitable for the mass production of organic electroluminescent devices.

In addition, hybrid methods are possible, in which, for example, one or more layers are applied from solution and one or more further layers are applied by vapour deposition.

These methods are known in general terms to those skilled in the art and can be applied to organic electroluminescent devices.

The invention therefore further provides a process for producing the organic electroluminescent device of the invention as described above or described as preferred, characterized in that the light-emitting layer is applied by gas phase deposition, especially by a sublimation method and/or by an OVPD (organic vapour phase deposition) method and/or with the aid of a carrier gas sublimation, or from solution, especially by spin-coating or by a printing method.

In the case of production by means of gas phase deposition, there are in principle two ways in which the light-emitting layer of the invention can be applied or vapour-deposited onto any substrate or the prior layer. Firstly, the materials used can each be initially charged in a material source and ultimately evaporated from the different material sources (“co-evaporation”). Secondly, the various materials can be premixed (premix systems) and the mixture can be initially charged in a single material source from which it is ultimately evaporated (“premix evaporation”). In this way, it is possible in a simple and rapid manner to achieve the vapour deposition of the light-emitting layer with homogeneous distribution of the components without the need for precise actuation of a multitude of material sources.

The invention accordingly further provides a process for producing the device of the invention, characterized in that the at least one compound of the formula (1) as described above or described as preferred and the at least one compound of the formula (2) as described above or described as preferred are deposited from the gas phase successively or simultaneously from at least two material sources, optionally with the at least one phosphorescent emitter as described above or described as preferred, and form the light-emitting layer.

In a preferred embodiment of the present invention, the light-emitting layer is applied by means of gas phase deposition, wherein the constituents of the composition are premixed and evaporated from a single material source.

The invention accordingly further provides a process for producing the device of the invention, characterized in that the at least one compound of the formula (1) and the at least one compound of the formula (2) are deposited from the gas phase as a mixture, successively or simultaneously with the at least one phosphorescent emitter, and form the light-emitting layer.

The invention further provides a process for producing the device of the invention, as described above or described as preferred, characterized in that the at least one compound of the formula (1) and the at least one compound of the formula (2), as described above or described as preferred, are applied from solution together with the at least one phosphorescent emitter in order to form the light-emitting layer.

The devices of the invention feature the following surprising advantages over the prior art:

The use of the described material combination of host materials 1 and 2, as described above, especially leads to an increase in the lifetime of the devices, with otherwise comparable performance data of the devices.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Any feature disclosed in the present invention, unless stated otherwise, should therefore be considered as an example from a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

The technical teaching disclosed with the present invention may be abstracted and combined with other examples.

The invention is illustrated in more detail by the examples which follow, without any intention of restricting it thereby.

General Methods:

In all quantum-chemical calculations, the Gaussian16 (Rev. B. 01) software package is used. The neutral singlet ground state is optimized at the B3LYP/6-31G(d) level. HOMO and LUMO values are determined at the B3LYP/6-31G(d) level for the B3LYP/6-31G(d)-optimized ground state energy. Then TD-DFT singlet and triplet excitations (vertical excitations) are calculated by the same method (B3LYP/6-31G(d)) and with the optimized ground state geometry. The standard settings for SCF and gradient convergence are used.

From the energy calculation, the HOMO is obtained as the last orbital occupied by two electrons (alpha occ. eigenvalues) and LUMO as the first unoccupied orbital (alpha virt. eigenvalues) in Hartree units, where HEh and LEh represent the HOMO energy in Hartree units and the LUMO energy in Hartree units respectively. This is used to determine the HOMO and LUMO value in electron volts, calibrated by cyclic voltammetry measurements, as follows:

HOMOcorr=0.90603*HOMO−0.84836

LUMOcorr=0.99687*LUMO−0.72445

The triplet level T1 of a material is defined as the relative excitation energy (in eV) of the triplet state having the lowest energy which is found by the quantum-chemical energy calculation.

The singlet level S1 of a material is defined as the relative excitation energy (in eV) of the singlet state having the second-lowest energy which is found by the quantum-chemical energy calculation.

The energetically lowest singlet state is referred to as S0.

The method described herein is independent of the software package used and always gives the same results. Examples of frequently utilized programs for this purpose are “Gaussian09” (Gaussian Inc.) and Q-Chem 4.1 (Q-Chem, Inc.). In the present case, the energies are calculated using the software package “Gaussian16 (Rev. B. 01)”.

### EXAMPLE 1: PRODUCTION OF THE OLEDS

The examples which follow (see tables 8 to 10) present the use of the material combinations of the invention in OLEDs by comparison with material combinations from the prior art.

Pretreatment for Examples V1 to V15 and E1a to E5i and E6a-E15a:

Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm. The exact structure of the OLEDs can be found in table 8. The materials required for production of the OLEDs, if they have not already been described before, are shown in table 10. The device data of the OLEDs are listed in table 9.

Examples V1 to V15 are comparative examples. Examples E1a to E5i and E6a-E15a show data for OLEDs of the invention.

All materials are applied by thermal vapour deposition in a vacuum chamber. In this case, the emission layer always consists of at least two matrix materials and an emitting dopant (emitter) which is added to the matrix material(s) in a particular proportion by volume by co-evaporation. Details given in such a form as E3:H3:TE2 (32%:60%:8%) mean here that the material E3 is present in the layer in a proportion by volume of 32%, H3 in a proportion of 60% and TE2 in a proportion of 8%. Analogously, the electron transport layer may also consist of a mixture of two materials.

The electroluminescence spectra are determined at a luminance of 1000 cd/m², and the CIE 1931 x and y colour coordinates are calculated therefrom. The parameter U10 in table 9 refers to the voltage which is required for a current density of 10 mA/cm². EQE10 denotes the external quantum efficiency which is attained at 10 mA/cm².

The lifetime LT is defined as the time after which luminance, measured in cd/m² in forward direction, drops from the starting luminance to a certain proportion L1 in the course of operation with constant current density jo. A figure of L1=80% in table 9 means that the lifetime reported in the LT column corresponds to the time after which luminance in cd/m² falls to 80% of its starting value.

Use of Mixtures of the Invention in OLEDs

The material combinations of the invention are used in examples E1a-k, E2a-k, E3a-k, E4a-k, E5a-i, E6a-E15a as matrix materials in the emission layer of green-phosphorescing OLEDs. As a comparison with the prior art, materials E55, E56, E57, E58, E59 and BCbz1 to BCbz6 are used in comparative examples V1 to V15. The combination of E58 with H9 in a light-emitting layer is disclosed, for example, in KR20180012499.

On comparison of the inventive examples with the corresponding comparative examples, it is clearly apparent that the inventive examples each show a distinct advantage in device lifetime, with otherwise comparable performance data of the OLEDs.

TABLE 8
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| V1 | SpMA1:PD1 | SpMA1 | SpMA2 | E55:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1a | SpMA1:PD1 | SpMA1 | SpMA2 | E3:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1b | SpMA1:PD1 | SpMA1 | SpMA2 | E5:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1c | SpMA1:PD1 | SpMA1 | SpMA2 | E18:H7:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1d | SpMA1:PD1 | SpMA1 | SpMA2 | E42:H5:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1e | SpMA1:PD1 | SpMA1 | SpMA2 | E48:H13:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1f | SpMA1:PD1 | SpMA1 | SpMA2 | E54:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1g | SpMA1:PD1 | SpMA1 | SpMA2 | E40:H5:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1h | SpMA1:PD1 | SpMA1 | SpMA2 | E34:H18:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1i | SpMA1:PD1 | SpMA1 | SpMA2 | E32:H18:TE2 | ST2 | ST2:LiQ | LiQ |
| | (95%:5%) | 200 nm | 20 nm | (22%:70%:8%) | 5 nm | (50%:50%) | 1 nm |
| | 20 nm | | | 40 nm | | 30 nm | |
| E1j | SpMA1:PD1 | SpMA1 | SpMA2 | E38:H4:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E1k | SpMA1:PD1 | SpMA1 | SpMA2 | E35:H6:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V2 | SpMA1:PD1 | SpMA1 | SpMA2 | E56:H3:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2a | SpMA1:PD1 | SpMA1 | SpMA2 | E29:H3:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2b | SpMA1:PD1 | SpMA1 | SpMA2 | E26:H15:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2c | SpMA1:PD1 | SpMA1 | SpMA2 | E25:H4:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2d | SpMA1:PD1 | SpMA1 | SpMA2 | E23:H20:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2e | SpMA1:PD1 | SpMA1 | SpMA2 | E11:H5:TE1 | ST2 | ST2:LiQ | LiQ |
| | (95%:5%) | 200 nm | 20 nm | (38%:50%:8%) | 5 nm | (50%:50%) | 1 nm |
| | 20 nm | | | 40 nm | | 30 nm | |
| E2f | SpMA1:PD1 | SpMA1 | SpMA2 | E4O:H3:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2g | SpMA1:PD1 | SpMA1 | SpMA2 | E19:H11:TE1 | ST2 | ST2:LiQ | LiQ |
| | (95%:5%) | 200 nm | 20 nm | (22%:70%:8%) | 5 nm | (50%:50%) | 1 nm |
| | 20 nm | | | 40 nm | | 30 nm | |
| E2h | SpMA1:PD1 | SpMA1 | SpMA2 | E44:H8:TE1 | ST2 | ST2:LiQ | LiQ |
| | (95%:5%) | 200 nm | 20 nm | (22%:70%:8%) | 5 nm | (50%:50%) | 1 nm |
| | 20 nm | | | 40 nm | | 30 nm | |
| E2i | SpMA1:PD1 | SpMA1 | SpMA2 | E4:H5:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2j | SpMA1:PD1 | SpMA1 | SpMA2 | E62:H5:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E2k | SpMA1:PD1 | SpMA1 | SpMA2 | E66:H20:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V3 | SpMA1:PD1 | SpMA1 | SpMA2 | E57:H5:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3a | SpMA1:PD1 | SpMA1 | SpMA2 | E46:H5:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3b | SpMA1:PD1 | SpMA1 | SpMA2 | E2:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3c | SpMA1:PD1 | SpMA1 | SpMA2 | E43:H1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3d | SpMA1:PD1 | SpMA1 | SpMA2 | E36:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3e | SpMA1:PD1 | SpMA1 | SpMA2 | E41:H12:TE2 | ST2 | ST2:LiQ | LiQ |
| | (95%:5%) | 200 nm | 20 nm | (22%:70%:8%) | 5 nm | (50%:50%) | 1 nm |
| | 20 nm | | | 40 nm | | 30 nm | |
| E3f | SpMA1:PD1 | SpMA1 | SpMA2 | E33:H21:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3g | SpMA1:PD1 | SpMA1 | SpMA2 | E8:H11:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3h | SpMA1:PD1 | SpMA1 | SpMA2 | E9:H10:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3i | SpMA1:PD1 | SpMA1 | SpMA2 | E13:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3j | SpMA1:PD1 | SpMA1 | SpMA2 | E16:H5:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E3k | SpMA1:PD1 | SpMA1 | SpMA2 | E63:H12:TE2 | ST2 | ST2:LiQ | LiQ |
| | (95%:5%) | 200 nm | 20 nm | (22%:70%:8%) | 5 nm | (50%:50%) | 1 nm |
| | 20 nm | | | 40 nm | | 30 nm | |
| V4 | SpMA1:PD1 | SpMA1 | SpMA2 | E58:H9:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4a | SpMA1:PD1 | SpMA1 | SpMA2 | E51:H9:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4b | SpMA1:PD1 | SpMA1 | SpMA2 | E40:H9:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4c | SpMA1:PD1 | SpMA1 | SpMA2 | E15:H10:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4d | SpMA1:PD1 | SpMA1 | SpMA2 | E31:H16:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4e | SpMA1:PD1 | SpMA1 | SpMA2 | E50:H3:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4f | SpMA1:PD1 | SpMA1 | SpMA2 | E24:H3:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4g | SpMA1:PD1 | SpMA1 | SpMA2 | E3O:H5:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4h | SpMA1:PD1 | SpMA1 | SpMA2 | E37:H4:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4i | SpMA1:PD1 | SpMA1 | SpMA2 | E14:H3:TE1 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E4j | SpMA1:PD1 | SpMA1 | SpMA2 | E39:H12:TE1 | ST2 | ST2:LiQ | LiQ |
| | (95%:5%) | 200 nm | 20 nm | (30%:58%:12%) | 5 nm | (50%:50%) | 1 nm |
| | 20 nm | | | 40 nm | | 30 nm | |
| E4k | SpMA1:PD1 | SpMA1 | SpMA2 | E65:H12:TE1 | ST2 | ST2:LiQ | LiQ |
| | (95%:5%) | 200 nm | 20 nm | (30%:58%:12%) | 5 nm | (50%:50%) | 1 nm |
| | 20 nm | | | 40 nm | | 30 nm | |
| V5 | SpMA1:PD1 | SpMA1 | SpMA2 | E59:H1:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5a | SpMA1:PD1 | SpMA1 | SpMA2 | E40:H1:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5b | SpMA1:PD1 | SpMA1 | SpMA2 | E35:H1:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5c | SpMA1:PD1 | SpMA1 | SpMA2 | E22:H4:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5d | SpMA1:PD1 | SpMA1 | SpMA2 | E10:H8:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5e | SpMA1:PD1 | SpMA1 | SpMA2 | E38:H11:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5f | SpMA1:PD1 | SpMA1 | SpMA2 | E53:H12:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5g | SpMA1:PD1 | SpMA1 | SpMA2 | E41:H3:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5h | SpMA1:PD1 | SpMA1 | SpMA2 | E40:H3:TE3 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E5i | SpMA1:PD1 | SpMA1 | SpMA2 | E40:H3:TE4 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| V6 | SpMA1:PD1 | SpMA1 | SpMA2 | E60:BCbz4:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E6a | SpMA1:PD1 | SpMA1 | SpMA2 | E60:H4:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V7 | SpMA1:PD1 | SpMA1 | SpMA2 | E38:BCbz4:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E7a | SpMA1:PD1 | SpMA1 | SpMA2 | E38:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V8 | SpMA1:PD1 | SpMA1 | SpMA2 | E39:BCbz1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E8a | SpMA1:PD1 | SpMA1 | SpMA2 | E39:H6:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V9 | SpMA1:PD1 | SpMA1 | SpMA2 | E52:BCbz2:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E9a | SpMA1:PD1 | SpMA1 | SpMA2 | E52:H3:TE1 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V10 | SpMA1:PD1 | SpMA1 | SpMA2 | E44:BCbz3:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| E10a | SpMA1:PD1 | SpMA1 | SpMA2 | E44:H8:TE2 | ST2 | ST2:LiQ | LiQ |
| | (95%:5%) | 200 nm | 20 nm | (38%:50%:12%) | 5 nm | (50%:50%) | 1 nm |
| | 20 nm | | | 40 nm | | 30 nm | |
| V11 | SpMA1:PD1 | SpMA1 | SpMA2 | E61:BCbz5:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E11a | SpMA1:PD1 | SpMA1 | SpMA2 | E61:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V12 | SpMA1:PD1 | SpMA1 | SpMA2 | E61:BCbz6:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E12a | SpMA1:PD1 | SpMA1 | SpMA2 | E61:H5:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V13 | SpMA1:PD1 | SpMA1 | SpMA2 | E35:BCbz1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E13a | SpMA1:PD1 | SpMA1 | SpMA2 | E35:H8:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V14 | SpMA1:PD1 | SpMA1 | SpMA2 | E32:BCbz3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E14a | SpMA1:PD1 | SpMA1 | SpMA2 | E32:H11:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| V15 | SpMA1:PD1 | SpMA1 | SpMA2 | E69:BCbz1:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |
| E15a | SpMA1:PD1 | SpMA1 | SpMA2 | E69:H3:TE2 | ST2 | ST2:LiQ | LiQ |
| | (95%:5%) | 200 nm | 20 nm | (32%:60%:8%) | 5 nm | (50%:50%) | 1 nm |
| | 20 nm | | | 40 nm | | 30 nm | |

TABLE 9
Data of the OLEDs

| | | | CIE x/y at | | | |
|---|---|---|---|---|---|---|
| | U10 | EQE10 | 1000 | j₀ | L1 | LT |
| Ex. | (V) | (%) | cd/m² | (mA/cm²) | (%) | (h) |
| V1 | 4.5 | 21.6 | 0.35/0.63 | 40 | 80 | 390 |
| E1a | 4.4 | 23.2 | 0.35/0.63 | 40 | 80 | 505 |
| E1b | 4.3 | 23.5 | 0.35/0.63 | 40 | 80 | 845 |
| E1c | 4.7 | 22.4 | 0.35/0.63 | 40 | 80 | 590 |
| E1d | 4.4 | 23.6 | 0.35/0.63 | 40 | 80 | 920 |
| E1e | 4.3 | 23.9 | 0.35/0.63 | 40 | 80 | 530 |
| E1f | 4.3 | 22.8 | 0.35/0.63 | 40 | 80 | 705 |
| E1g | 4.4 | 21.4 | 0.35/0.63 | 40 | 80 | 1080 |
| E1h | 4.5 | 22.1 | 0.35/0.63 | 40 | 80 | 700 |
| E1i | 4.4 | 22.5 | 0.35/0.63 | 40 | 80 | 1060 |
| E1j | 4.4 | 23.0 | 0.35/0.63 | 40 | 80 | 840 |
| E1k | 4.3 | 22.8 | 0.35/0.63 | 40 | 80 | 960 |
| V2 | 5.1 | 18.1 | 0.34/0.62 | 40 | 80 | 610 |
| E2a | 5.1 | 19.0 | 0.34/0.62 | 40 | 80 | 815 |
| E2b | 5.4 | 18.6 | 0.34/0.62 | 40 | 80 | 715 |
| E2c | 5.3 | 19.2 | 0.33/0.63 | 40 | 80 | 730 |
| E2d | 5.1 | 17.5 | 0.34/0.62 | 40 | 80 | 790 |
| E2e | 5.2 | 19.1 | 0.34/0.62 | 40 | 80 | 660 |
| E2f | 5.4 | 19.6 | 0.34/0.62 | 40 | 80 | 675 |
| E2g | 5.3 | 19.4 | 0.34/0.62 | 40 | 80 | 840 |
| E2h | 5.2 | 18.7 | 0.33/0.63 | 40 | 80 | 800 |
| E2i | 5.1 | 18.5 | 0.33/0.63 | 40 | 80 | 975 |
| E2j | 5.5 | 19.3 | 0.34/0.62 | 40 | 80 | 690 |
| E2k | 5.3 | 18.1 | 0.34/0.62 | 40 | 80 | 715 |
| V3 | 4.4 | 23.4 | 0.35/0.62 | 40 | 80 | 410 |
| E3a | 4.6 | 23.0 | 0.34/0.63 | 40 | 80 | 950 |
| E3b | 4.5 | 23.2 | 0.35/0.63 | 40 | 80 | 605 |
| E3c | 4.7 | 22.6 | 0.35/0.63 | 40 | 80 | 590 |
| E3d | 4.6 | 21.6 | 0.35/0.62 | 40 | 80 | 760 |
| E3e | 4.5 | 23.0 | 0.34/0.63 | 40 | 80 | 1020 |
| E3f | 4.4 | 22.1 | 0.35/0.62 | 40 | 80 | 815 |
| E3g | 4.6 | 22.4 | 0.35/0.62 | 40 | 80 | 840 |
| E3h | 4.5 | 22.8 | 0.35/0.62 | 40 | 80 | 755 |
| E3i | 4.7 | 22.0 | 0.34/0.63 | 40 | 80 | 490 |
| E3j | 4.4 | 22.2 | 0.35/0.63 | 40 | 80 | 780 |
| E3k | 4.7 | 22.8 | 0.35/0.63 | 40 | 80 | 1035 |
| V4 | 4.9 | 18.0 | 0.34/0.62 | 40 | 80 | 665 |
| E4a | 4.8 | 18.5 | 0.33/0.63 | 40 | 80 | 960 |
| E4b | 4.6 | 18.1 | 0.33/0.63 | 40 | 80 | 1760 |
| E4c | 4.7 | 19.3 | 0.33/0.63 | 40 | 80 | 1330 |
| E4d | 4.9 | 19.5 | 0.33/0.63 | 40 | 80 | 1540 |
| E4e | 4.6 | 19.3 | 0.33/0.63 | 40 | 80 | 1150 |
| E4f | 4.9 | 18.7 | 0.34/0.62 | 40 | 80 | 1030 |
| E4g | 4.8 | 19.0 | 0.33/0.63 | 40 | 80 | 885 |
| E4h | 4.9 | 19.4 | 0.34/0.62 | 40 | 80 | 1070 |
| E4i | 4.7 | 18.7 | 0.34/0.62 | 40 | 80 | 885 |
| E4j | 4.8 | 18.9 | 0.34/0.62 | 40 | 80 | 1610 |
| E4k | 5.0 | 18.8 | 0.34/0.63 | 40 | 80 | 1450 |
| V5 | 4.6 | 20.7 | 0.35/0.63 | 40 | 80 | 810 |
| E5a | 4.6 | 20.3 | 0.34/0.63 | 40 | 80 | 1330 |
| E5b | 4.5 | 21.2 | 0.34/0.63 | 40 | 80 | 1145 |
| E5c | 4.7 | 20.7 | 0.35/0.63 | 40 | 80 | 1020 |
| E5d | 4.7 | 20.4 | 0.35/0.63 | 40 | 80 | 950 |
| E5e | 4.7 | 20.5 | 0.35/0.63 | 40 | 80 | 1080 |
| E5f | 4.7 | 20.3 | 0.35/0.63 | 40 | 80 | 1160 |
| E5g | 4.5 | 21.0 | 0.35/0.63 | 40 | 80 | 1250 |
| E5h | 4.9 | 20.1 | 0.35/0.63 | 40 | 80 | 1475 |
| E5i | 4.8 | 19.3 | 0.35/0.63 | 40 | 80 | 960 |
| V6 | 4.4 | 22.4 | 0.34/0.63 | 40 | 80 | 615 |
| E6a | 4.5 | 22.2 | 0.35/0.63 | 40 | 80 | 680 |
| V7 | 4.3 | 23.6 | 0.35/0.63 | 40 | 80 | 735 |
| E7a | 4.4 | 23.0 | 0.35/0.63 | 40 | 80 | 870 |
| V8 | 4.3 | 23.3 | 0.35/0.63 | 40 | 80 | 820 |
| E8a | 4.4 | 22.9 | 0.35/0.63 | 40 | 80 | 910 |
| V9 | 5.0 | 18.9 | 0.34/0.62 | 40 | 80 | 640 |
| E9a | 5.0 | 19.4 | 0.34/0.62 | 40 | 80 | 910 |
| V10 | 4.4 | 20.8 | 0.35/0.63 | 40 | 80 | 915 |
| E10a | 4.6 | 20.4 | 0.34/0.63 | 40 | 80 | 1080 |
| V11 | 4.3 | 23.7 | 0.34/0.63 | 40 | 80 | 755 |
| E11a | 4.3 | 23.2 | 0.34/0.63 | 40 | 80 | 875 |
| V12 | 4.1 | 23.6 | 0.34/0.63 | 40 | 80 | 735 |
| E12a | 4.3 | 23.4 | 0.34/0.63 | 40 | 80 | 900 |
| V13 | 4.2 | 23.5 | 0.35/0.63 | 40 | 80 | 845 |
| E13a | 4.4 | 23.0 | 0.35/0.63 | 40 | 80 | 930 |
| V14 | 4.2 | 23.6 | 0.35/0.63 | 40 | 80 | 875 |
| E14a | 4.3 | 22.9 | 0.35/0.63 | 40 | 80 | 1005 |
| V15 | 4.1 | 23.9 | 0.34/0.63 | 40 | 80 | 960 |
| E15a | 4.3 | 23.8 | 0.35/0.63 | 40 | 80 | 1115 |

TABLE 10
Structural formulae of the materials of the OLEDs used, if not
already described before:

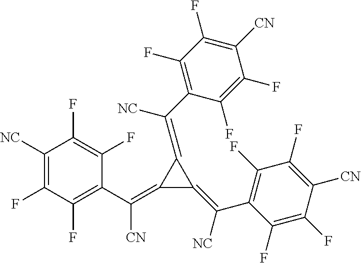 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

PD1 (CAS Reg. No. 1224447-88-4)

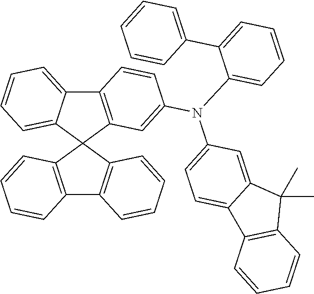 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

SpMA1

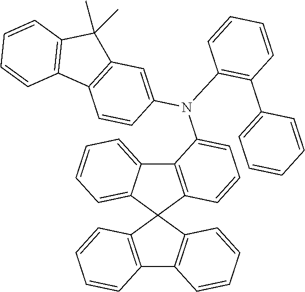 [SMILES: CC1(C)c2ccccc2-c2ccc(N(c3ccccc3-c3ccccc3)c3cccc4c3-c3ccccc3C43c4ccccc4-c4ccccc43)cc21]

SpMA2

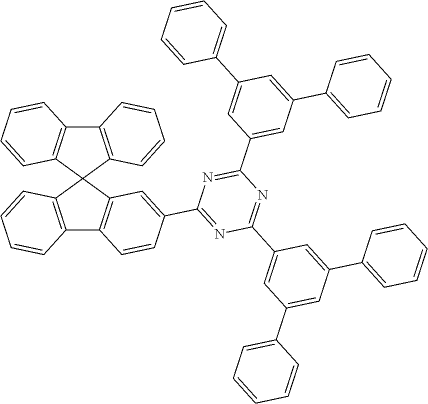 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

ST2

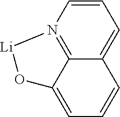

LiQ

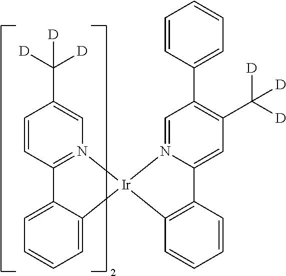

TE1

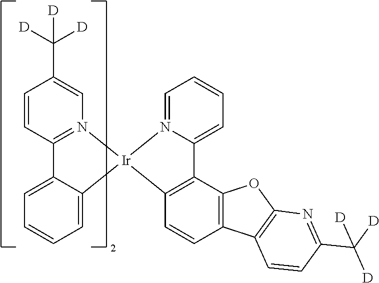

TE2

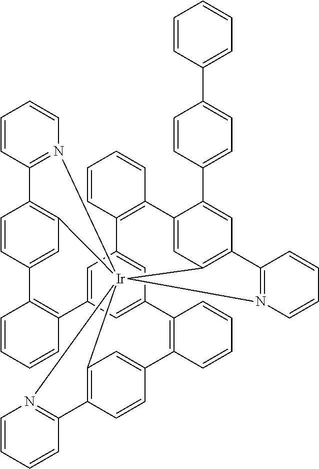

TE3

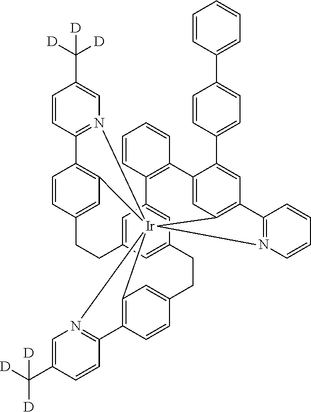

TE4

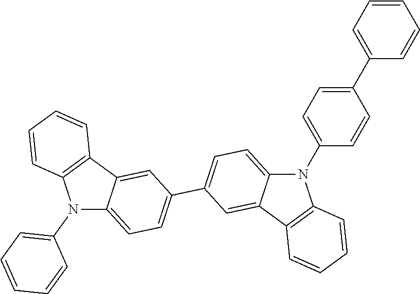 [SMILES: c1ccc(-c2ccc(-n3c4ccccc4c4cc(-c5ccc6c(c5)c5ccccc5n6-c5ccccc5)ccc43)cc2)cc1]

BCbz1

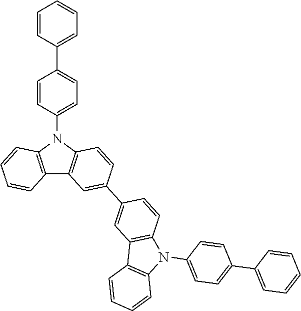 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

BCbz2

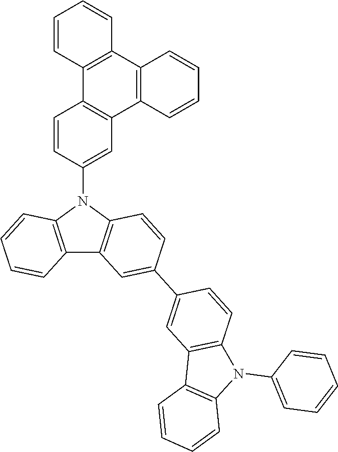 [SMILES: c1ccc(-n2c3ccccc3c3cc(-c4ccc5c(c4)c4ccccc4n5-c4ccc5c6ccccc6c6ccccc6c5c4)ccc32)cc1]

BCbz3

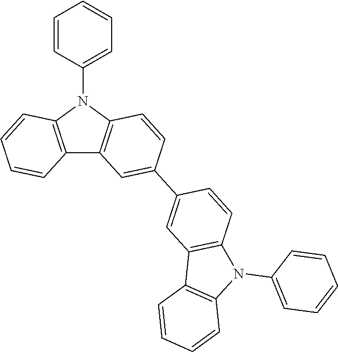 [SMILES: c1ccc(-n2c3ccccc3c3cc(-c4ccc5c(c4)c4ccccc4n5-c4ccccc4)ccc32)cc1]

BCbz4

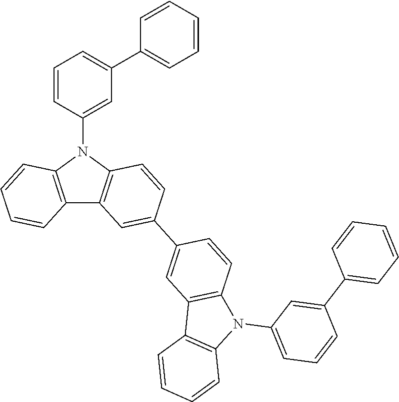 [SMILES: c1ccc(-c2cccc(-n3c4ccccc4c4cc(-c5ccc6c(c5)c5ccccc5n6-c5cccc(-c6ccccc6)c5)ccc43)c2)cc1]

BCbz5

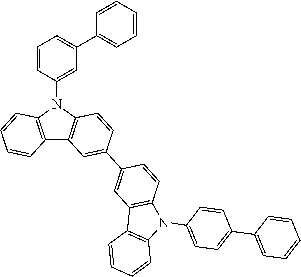 [SMILES: c1ccc(-c2ccc(-n3c4ccccc4c4cc(-c5ccc6c(c5)c5ccccc5n6-c5cccc(-c6ccccc6)c5)ccc43)cc2)cc1]

BCbz6

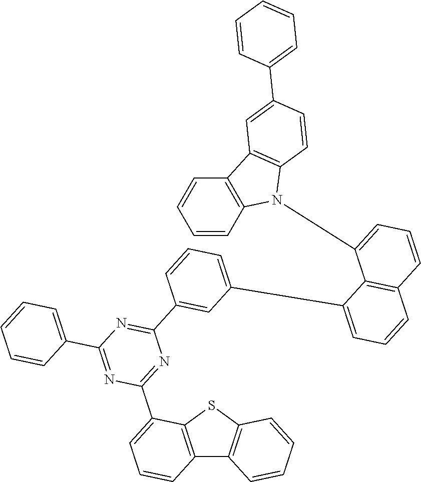 [SMILES: c1ccc(-c2ccc3c(c2)c2ccccc2n3-c2cccc3cccc(-c4cccc(-c5nc(-c6ccccc6)nc(-c6cccc7c6sc6ccccc67)n5)c4)c23)cc1]

E55

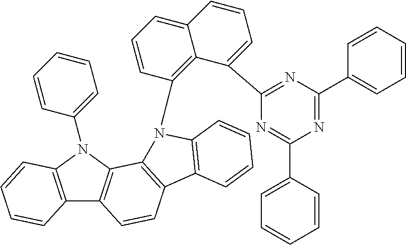 [SMILES: c1ccc(-c2nc(-c3ccccc3)nc(-c3cccc4cccc(-n5c6ccccc6c6ccc7c8ccccc8n(-c8ccccc8)c7c65)c34)n2)cc1]

E56

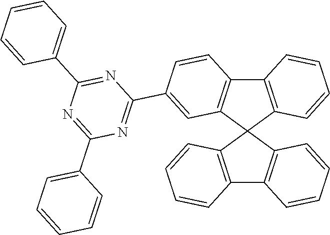 [SMILES: c1ccc(-c2nc(-c3ccccc3)nc(-c3ccc4c(c3)C3(c5ccccc5-c5ccccc53)c3ccccc3-4)n2)cc1]

E57

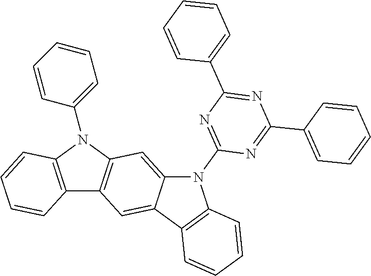 [SMILES: c1ccc(-c2nc(-c3ccccc3)nc(-n3c4ccccc4c4cc5c6ccccc6n(-c6ccccc6)c5cc43)n2)cc1]

E58

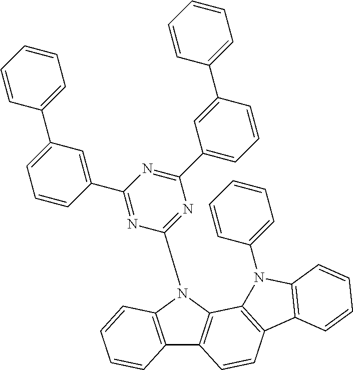 [SMILES: c1ccc(-c2cccc(-c3nc(-c4cccc(-c5ccccc5)c4)nc(-n4c5ccccc5c5ccc6c7ccccc7n(-c7ccccc7)c6c54)n3)c2)cc1]

E59

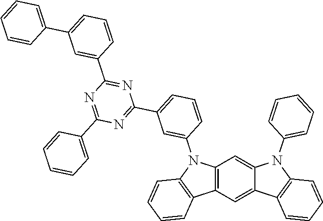 [SMILES: c1ccc(-c2cccc(-c3nc(-c4ccccc4)nc(-c4cccc(-n5c6ccccc6c6cc7c8ccccc8n(-c8ccccc8)c7cc65)c4)n3)c2)cc1]

E60.

E55 and E56 are described in WO2015014435; E57 is described in WO2011088877; E58 is described in KR20180012499; E59 is described in US20100187977; E60 is described in US20170117488.

The syntheses which follow, unless stated otherwise, are conducted under a protective gas atmosphere in dried solvents. The solvents and reagents can be purchased, for example, from Sigma-ALDRICH or ABCR. The respective figures in square brackets or the numbers quoted for individual compounds relate to the CAS numbers of the compounds known from the literature.

### Preparation of the Compounds

E5 (117):

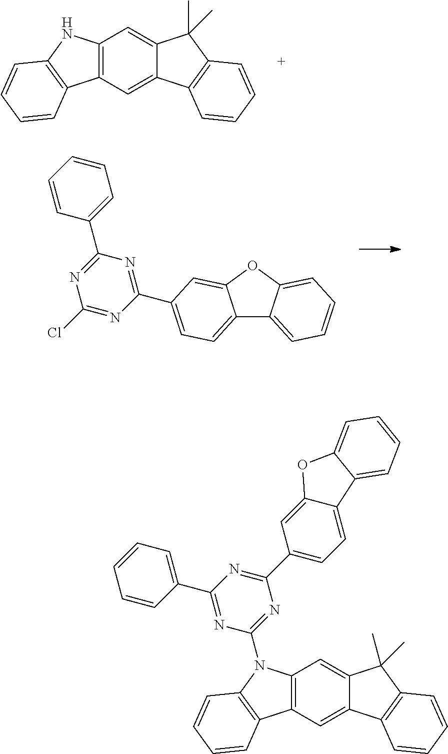

7,7-Dimethyl-5H-indeno[2,1-b]carbazole [CAS-1257220-47-5] (28.34 g, 100.0 mmol) is initially charged under inert atmosphere in 600 ml of dried DMF. At room temperature, sodium hydride suspension (60% in paraffin oil) (4.19 g, 105.0 mmol) is added gradually, and the mixture is stirred at room temperature for 1 h. Subsequently, 2-chloro-4-dibenzofuran-3-yl-6-phenyl-1,3,5-triazine [2142681-84-1] (37.57 g, 105.0 mmol) is added cautiously, and the reaction mixture is stirred at room temperature overnight. 500 ml of water is added dropwise and the mixture is stirred for a further 1 h, then the solids are filtered off with suction and washed 3× with 250 ml of water and 3× with 250 ml of ethanol. The crude product is subjected to basic hot extraction twice with toluene/heptane (3:1) over aluminium oxide, then recrystallized three times from ethyl acetate and finally sublimed under high vacuum. Yield: 27.4 g (45.3 mmol, 45%); purity: >99.9% by HPLC.

The following compounds can be prepared analogously: Purification can also be effected using column chromatography, or recrystallization or hot extraction using other standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methyl pyrrolidone, etc.

| Reactant 1 | Reactant 2 | Product | Yield |
|---|---|---|---|
| 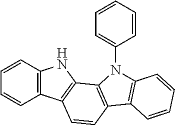 [SMILES: c1ccc(-n2c3ccccc3c3ccc4c5ccccc5[nH]c4c32)cc1] CAS-1024598-06-8 | 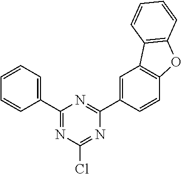 [SMILES: Clc1nc(-c2ccccc2)nc(-c2ccc3oc4ccccc4c3c2)n1] CAS-1618107-00-8 | 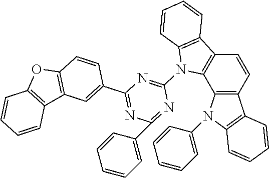 [SMILES: c1ccc(-c2nc(-c3ccc4oc5ccccc5c4c3)nc(-n3c4ccccc4c4ccc5c6ccccc6n(-c6ccccc6)c5c43)n2)cc1] E2 (63) | 46% |
| 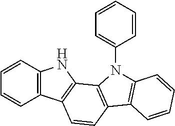 [SMILES: c1ccc(-n2c3ccccc3c3ccc4c5ccccc5[nH]c4c32)cc1] CAS-1024598-06-8 | 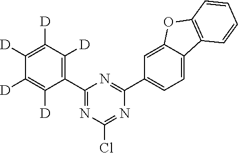 [SMILES: [2H]c1c([2H])c([2H])c(-c2nc(Cl)nc(-c3ccc4c(c3)oc3ccccc34)n2)c([2H])c1[2H]] CAS- 2226747-65-3 | 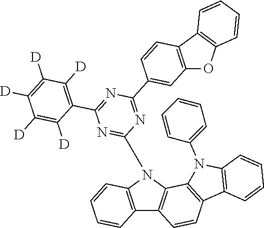 [SMILES: [2H]c1c([2H])c([2H])c(-c2nc(-c3ccc4c(c3)oc3ccccc34)nc(-n3c4ccccc4c4ccc5c6ccccc6n(-c6ccccc6)c5c43)n2)c([2H])c1[2H]] | 41% |
| | | 1278 | |
| 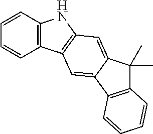 [SMILES: CC1(C)c2ccccc2-c2cc3c(cc21)[nH]c1ccccc13] CAS-1257220-47-5 | 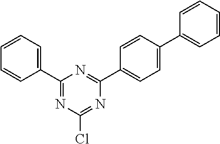 [SMILES: Clc1nc(-c2ccccc2)nc(-c2ccc(-c3ccccc3)cc2)n1] CAS-1472062-94-4 | 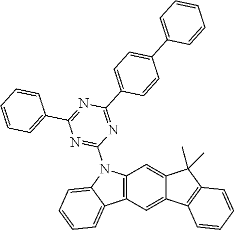 [SMILES: CC1(C)c2ccccc2-c2cc3c4ccccc4n(-c4nc(-c5ccccc5)nc(-c5ccc(-c6ccccc6)cc5)n4)c3cc21] | 55% |
| | | E39 (766) | |
| 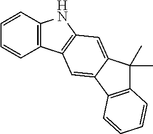 [SMILES: CC1(C)c2ccccc2-c2cc3c(cc21)[nH]c1ccccc13] CAS-1257220-47-5 | 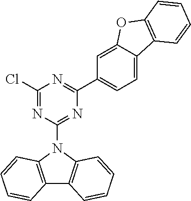 [SMILES: Clc1nc(-c2ccc3c(c2)oc2ccccc23)nc(-n2c3ccccc3c3ccccc32)n1] CAS-2260688-95-5 | 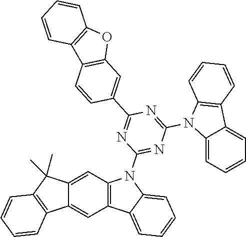 [SMILES: CC1(C)c2ccccc2-c2cc3c4ccccc4n(-c4nc(-c5ccc6c(c5)oc5ccccc56)nc(-n5c6ccccc6c6ccccc65)n4)c3cc21] | 47% |
| | | E44 (897) | |
| 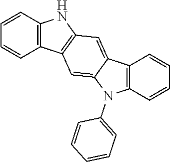 [SMILES: c1ccc(-n2c3ccccc3c3cc4[nH]c5ccccc5c4cc32)cc1] CAS-1316311-27-9 | 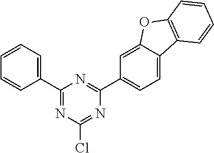 [SMILES: Clc1nc(-c2ccccc2)nc(-c2ccc3c(c2)oc2ccccc23)n1] CAS-2142681-84-1 | 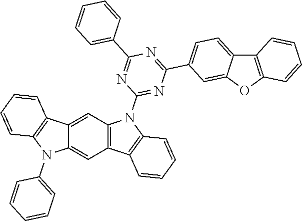 [SMILES: c1ccc(-c2nc(-c3ccc4c(c3)oc3ccccc34)nc(-n3c4ccccc4c4cc5c(cc43)c3ccccc3n5-c3ccccc3)n2)cc1] | 40% |
| | | E11 (159) | |
| 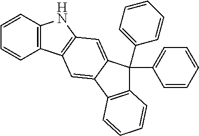 [SMILES: c1ccc(C2(c3ccccc3)c3ccccc3-c3cc4c(cc32)[nH]c2ccccc24)cc1] CAS-1257220-52-2 | 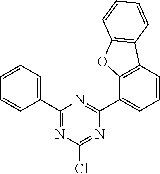 [SMILES: Clc1nc(-c2ccccc2)nc(-c2cccc3c2oc2ccccc23)n1] CAS-1472729-25-1 | 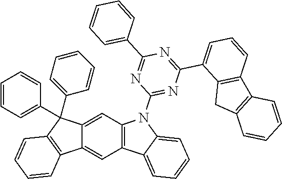 [SMILES: c1ccc(-c2nc(-c3cccc4c3Cc3ccccc3-4)nc(-n3c4ccccc4c4cc5c(cc43)C(c3ccccc3)(c3ccccc3)c3ccccc3-5)n2)cc1] E14 (194) | 44% |
| 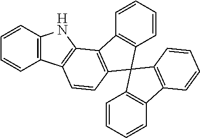 [SMILES: c1ccc2c(c1)-c1ccccc1C21c2ccccc2-c2c1ccc1c2[nH]c2ccccc21] CAS-1615703-28- | 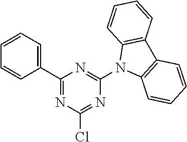 [SMILES: Clc1nc(-c2ccccc2)nc(-n2c3ccccc3c3ccccc32)n1] CAS-1268244-56-9 | 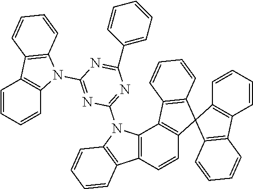 [SMILES: c1ccc(-c2nc(-n3c4ccccc4c4ccccc43)nc(-n3c4ccccc4c4ccc5c(c43)-c3ccccc3C53c4ccccc4-c4ccccc43)n2)cc1] | 38% |
| | | E54 (1237) | |
| 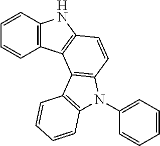 [SMILES: c1ccc(-n2c3ccccc3c3c4c(ccc32)[nH]c2ccccc24)cc1] CAS-1637752-63- | 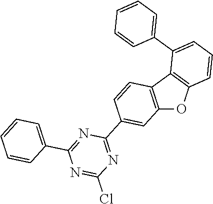 [SMILES: Clc1nc(-c2ccccc2)nc(-c2ccc3c(c2)oc2cccc(-c4ccccc4)c23)n1] CAS-2226747-84-6 | 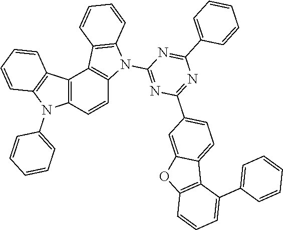 [SMILES: c1ccc(-c2nc(-c3ccc4c(c3)oc3cccc(-c5ccccc5)c34)nc(-n3c4ccccc4c4c5c6ccccc6n(-c6ccccc6)c5ccc43)n2)cc1] E23 (316) | 36% |
| 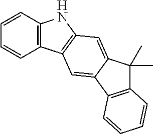 [SMILES: CC1(C)c2ccccc2-c2cc3c(cc21)[nH]c1ccccc13] CAS-1257220-47-5 | 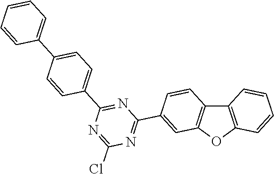 [SMILES: Clc1nc(-c2ccc(-c3ccccc3)cc2)nc(-c2ccc3c(c2)oc2ccccc23)n1] CAS-2170887-83-7 | 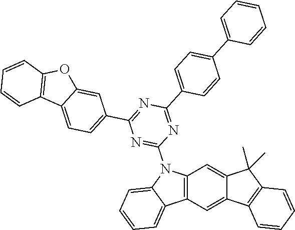 [SMILES: CC1(C)c2ccccc2-c2cc3c4ccccc4n(-c4nc(-c5ccc(-c6ccccc6)cc5)nc(-c5ccc6c(c5)oc5ccccc56)n4)c3cc21] | 41% |
| | | E32 (567) | |
| 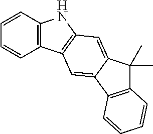 [SMILES: CC1(C)c2ccccc2-c2cc3c(cc21)[nH]c1ccccc13] CAS-1257220-47-5 | 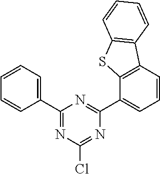 [SMILES: Clc1nc(-c2ccccc2)nc(-c2cccc3c2sc2ccccc23)n1] CAS-1476735-48-4 | 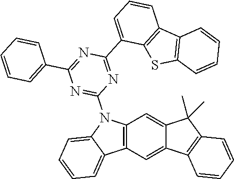 [SMILES: CC1(C)c2ccccc2-c2cc3c4ccccc4n(-c4nc(-c5ccccc5)nc(-c5cccc6c5sc5ccccc56)n4)c3cc21] E15 (223) | 55% |
| 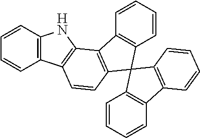 [SMILES: c1ccc2c(c1)-c1ccccc1C21c2ccccc2-c2c1ccc1c2[nH]c2ccccc21] CAS-1615703-28- | 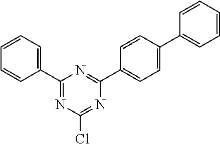 [SMILES: Clc1nc(-c2ccccc2)nc(-c2ccc(-c3ccccc3)cc2)n1] CAS-1472062-94-4 | 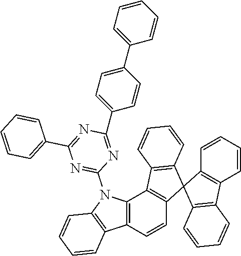 [SMILES: c1ccc(-c2ccc(-c3nc(-c4ccccc4)nc(-n4c5ccccc5c5ccc6c(c54)-c4ccccc4C64c5ccccc5-c5ccccc54)n3)cc2)cc1] | 45% |
| | | E41 (771) | |
| 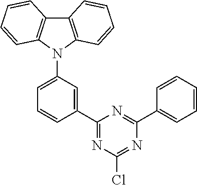 [SMILES: Clc1nc(-c2ccccc2)nc(-c2cccc(-n3c4ccccc4c4ccccc43)c2)n1] | 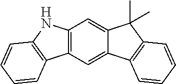 [SMILES: CC1(C)c2ccccc2-c2cc3c(cc21)[nH]c1ccccc13] | 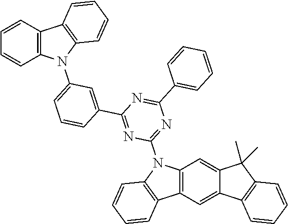 [SMILES: CC1(C)c2ccccc2-c2cc3c4ccccc4n(-c4nc(-c5ccccc5)nc(-c5cccc(-n6c7ccccc7c7ccccc76)c5)n4)c3cc21] | 52% |
| | | E61 | |

### Synthesis of E38

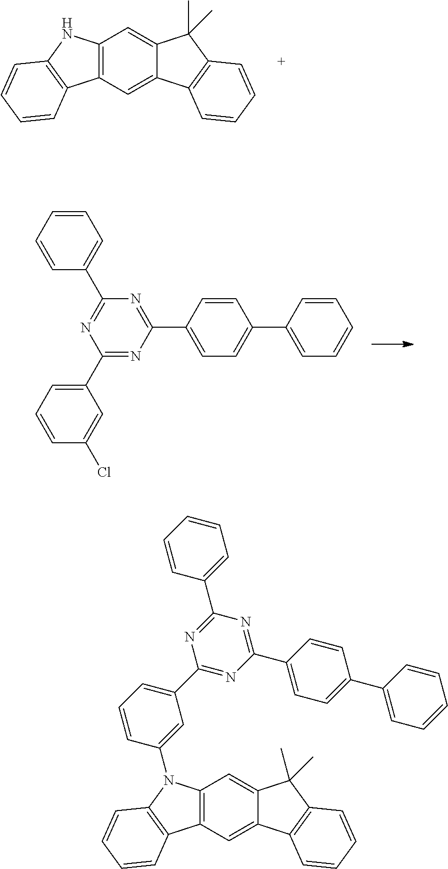 [SMILES: CC1(C)c2ccccc2-c2cc3c(cc21)[nH]c1ccccc13 ; Clc1cccc(-c2nc(-c3ccccc3)nc(-c3ccc(-c4ccccc4)cc3)n2)c1 ; CC1(C)c2ccccc2-c2cc3c4ccccc4n(-c4cccc(-c5nc(-c6ccccc6)nc(-c6ccc(-c7ccccc7)cc6)n5)c4)c3cc21]

An initial charge of 7,7-dimethyl-5H-indeno[2,1-b]carbazole [CAS-1257220-47-5] (28.34 g, 100.0 mmol), 2-[1,1′-biphenyl]-4-yl-4-(3-chlorophenyl)-6-phenyl-1,3,5-triazine [2085262-87-7] (46.19 g, 110 mmol) and sodium tert-butyloxide (19.22 g, 200 mmol) in toluene (900 ml) is inertized for 30 min. Subsequently, XPhos (3.28 g, 6.88 mmol) and Pd₂(dba)₃ (1.26 g, 1.38 mmol) are added successively and the reaction mixture is heated under reflux for 16 h. The mixture is worked up by extraction with toluene/water, the combined organic phases are dried over Na₂SO₄, and the filtrate is concentrated to dryness. The residue is suspended in ethanol (700 ml) and boiled under reflux for 2 h. The solids are filtered off with suction and washed with ethanol. The crude product is subjected to hot extraction three times with toluene/heptane (1:2), then recrystallized twice from ethyl acetate and finally sublimed under high vacuum. Yield: 34.6 g (51.9 mmol, 52%); purity: >99.9% by HPLC.

The following compounds can be prepared analogously: The catalyst system used here (palladium source and ligand) may also be Pd₂(dba)₃ with SPhos [657408-07-6] or Pd(OAc)₂ with S-Phos or Pd₂(dba)₃ with PtBu₃ or Pd(OAc)₂ with P^{t}Bu₃ (tBu means tert-butyl). Purification can also be effected using column chromatography, or recrystallization or hot extraction using other standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methylpyrrolidone, etc.

| Reactant 1 | Reactant 2 | Product | Yield |
|---|---|---|---|
| 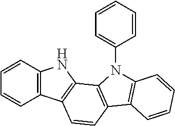 [SMILES: c1ccc(-n2c3ccccc3c3ccc4c5ccccc5[nH]c4c32)cc1] CAS-1024598-06-8 | 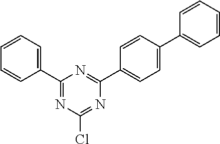 [SMILES: Clc1nc(-c2ccccc2)nc(-c2ccc(-c3ccccc3)cc2)n1] CAS-1472062-94-4 | 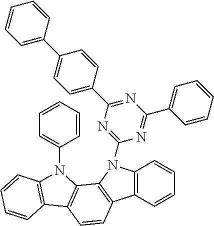 [SMILES: c1ccc(-c2ccc(-c3nc(-c4ccccc4)nc(-n4c5ccccc5c5ccc6c7ccccc7n(-c7ccccc7)c6c54)n3)cc2)cc1] | 28% |
| | | E40 (767) | |
| 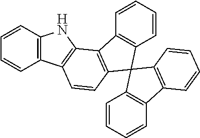 [SMILES: c1ccc2c(c1)-c1ccccc1C21c2ccccc2-c2c1ccc1c2[nH]c2ccccc21] CAS-1615703-28-0 | 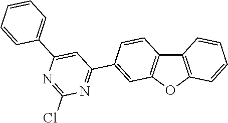 [SMILES: Clc1nc(-c2ccccc2)cc(-c2ccc3c(c2)oc2ccccc23)n1] CAS-2286234-09-9 | 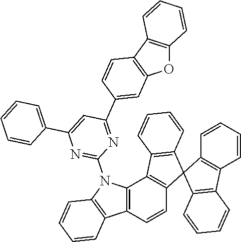 [SMILES: c1ccc(-c2cc(-c3ccc4c(c3)oc3ccccc34)nc(-n3c4ccccc4c4ccc5c(c43)-c3ccccc3C53c4ccccc4-c4ccccc43)n2)cc1] | 45% |
| | | E48 (1070) | |
| 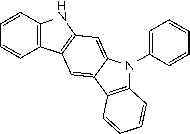 [SMILES: c1ccc(-n2c3ccccc3c3cc4c(cc32)[nH]c2ccccc24)cc1] CAS-1448296-00-1 | 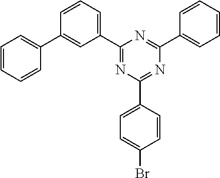 [SMILES: Brc1ccc(-c2nc(-c3ccccc3)nc(-c3cccc(-c4ccccc4)c3)n2)cc1] CAS-2102445-23-6 | 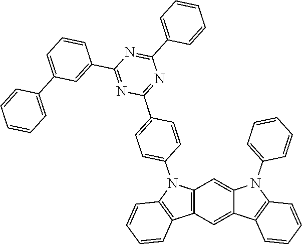 [SMILES: c1ccc(-c2cccc(-c3nc(-c4ccccc4)nc(-c4ccc(-n5c6ccccc6c6cc7c8ccccc8n(-c8ccccc8)c7cc65)cc4)n3)c2)cc1] | 56% |
| | | E43 (837) | |
| 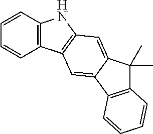 [SMILES: CC1(C)c2ccccc2-c2cc3c(cc21)[nH]c1ccccc13] CAS-1257220-47 | 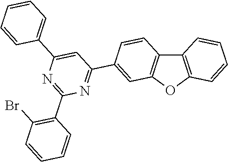 [SMILES: Brc1ccccc1-c1nc(-c2ccccc2)cc(-c2ccc3c(c2)oc2ccccc23)n1] | 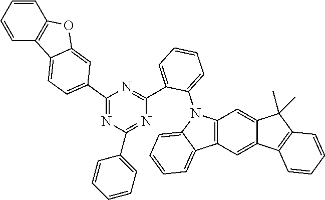 [SMILES: CC1(C)c2ccccc2-c2cc3c4ccccc4n(-c4ccccc4-c4nc(-c5ccccc5)nc(-c5ccc6c(c5)oc5ccccc56)n4)c3cc21] | 24% |
| | | E3 (111) | |
| 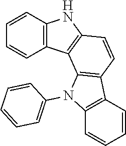 [SMILES: c1ccc(-n2c3ccccc3c3ccc4[nH]c5ccccc5c4c32)cc1] CAS-1346571-68-3 | 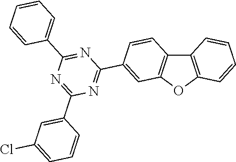 [SMILES: Clc1cccc(-c2nc(-c3ccccc3)nc(-c3ccc4c(c3)oc3ccccc34)n2)c1] CAS-2172889-30-2 | 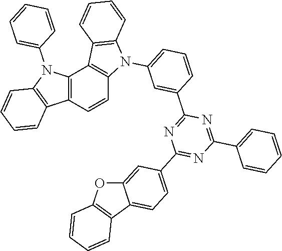 [SMILES: c1ccc(-c2nc(-c3cccc(-n4c5ccccc5c5c4ccc4c6ccccc6n(-c6ccccc6)c45)c3)nc(-c3ccc4c(c3)oc3ccccc34)n2)cc1] | 52% |
| | | E9 (149) | |
| 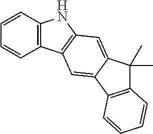 [SMILES: CC1(C)c2ccccc2-c2cc3c(cc21)[nH]c1ccccc13] CAS-1257220-47 | 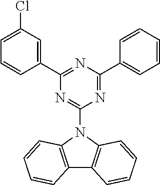 [SMILES: Clc1cccc(-c2nc(-c3ccccc3)nc(-n3c4ccccc4c4ccccc43)n2)c1] CAS-2260689-00-5 | 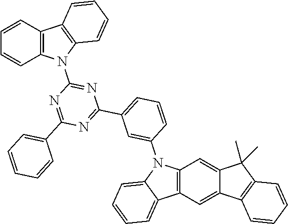 [SMILES: CC1(C)c2ccccc2-c2cc3c4ccccc4n(-c4cccc(-c5nc(-c6ccccc6)nc(-n6c7ccccc7c7ccccc76)n5)c4)c3cc21] | 58% |
| | | E51 (1228) | |
| 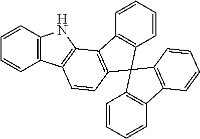 [SMILES: c1ccc2c(c1)-c1ccccc1C21c2ccccc2-c2c1ccc1c2[nH]c2ccccc21] CAS-1615703-28-0 | 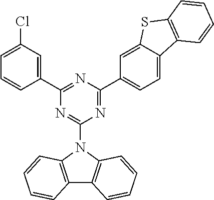 [SMILES: Clc1cccc(-c2nc(-c3ccc4c(c3)sc3ccccc34)nc(-n3c4ccccc4c4ccccc43)n2)c1] CAS-2260689-12-9 | 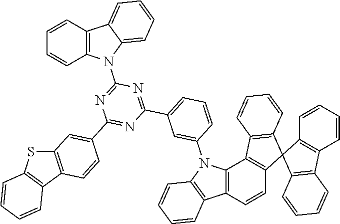 [SMILES: c1cc(-c2nc(-c3ccc4c(c3)sc3ccccc34)nc(-n3c4ccccc4c4ccccc43)n2)cc(-n2c3ccccc3c3ccc4c(c32)-c2ccccc2C42c3ccccc3-c3ccccc32)c1] E34 (619) | 50% |
| 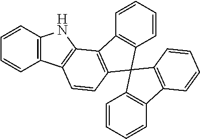 [SMILES: c1ccc2c(c1)-c1ccccc1C21c2ccccc2-c2c1ccc1c2[nH]c2ccccc21] CAS-1615703-28-0 | 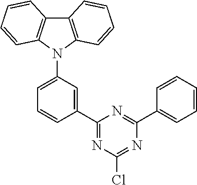 [SMILES: Clc1nc(-c2ccccc2)nc(-c2cccc(-n3c4ccccc4c4ccccc43)c2)n1] | 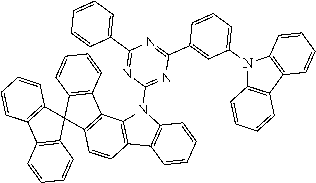 [SMILES: c1ccc(-c2nc(-c3cccc(-n4c5ccccc5c5ccccc54)c3)nc(-n3c4ccccc4c4ccc5c(c43)-c3ccccc3C53c4ccccc4-c4ccccc43)n2)cc1] | 36% |
| | | E63 (1294) | |
| 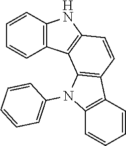 [SMILES: c1ccc(-n2c3ccccc3c3ccc4[nH]c5ccccc5c4c32)cc1] CAS-1346571-68-3 | 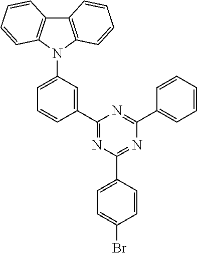 [SMILES: Brc1ccc(-c2nc(-c3ccccc3)nc(-c3cccc(-n4c5ccccc5c5ccccc54)c3)n2)cc1] | 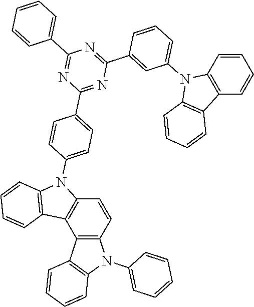 [SMILES: c1ccc(-c2nc(-c3ccc(-n4c5ccccc5c5c6c7ccccc7n(-c7ccccc7)c6ccc54)cc3)nc(-c3cccc(-n4c5ccccc5c5ccccc54)c3)n2)cc1] | 48% |
| | | E66 (1317) | |

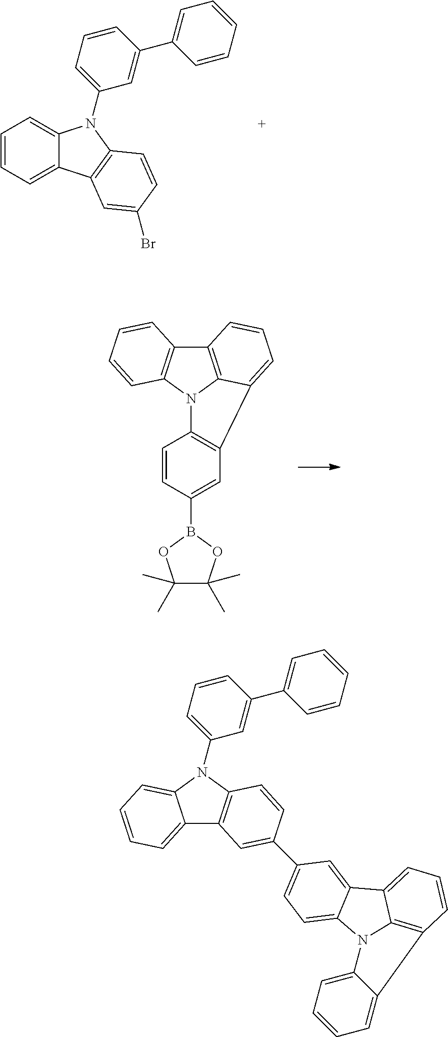 [SMILES: Brc1ccc2c(c1)c1ccccc1n2-c1cccc(-c2ccccc2)c1 ; CC1(C)OB(c2ccc3c(c2)c2cccc4c5ccccc5n3c42)OC1(C)C ; c1ccc(-c2cccc(-n3c4ccccc4c4cc(-c5ccc6c(c5)c5cccc7c8ccccc8n6c75)ccc43)c2)cc1]

To an initial charge of 9-[1,1′-biphenyl]-3-yl-3-bromo-9H-carbazole (59.88 g, 150.3 mmol) [CAS-1428551-28-3], 5-(4,4,5,5-tetramethyl-1,3,2-dioxaborolan-2-yl)indolo[3,2,1-jk]carbazole (51.1 g, 147.3 mmol) [CAS-1454807-26-1] in toluene (1200 ml), 1,4-dioxane (1200 ml) and water (600 ml) under inert atmosphere are added K₃PO₄ (95.7 g, 451 mmol), tri(ortho-tolyl)phosphine (2.33 g, 7.52 mmol) and Pd(OAc)₂ (840 mg, 3.76 mmol), and the mixture is stirred under reflux for 32 h. After cooling, the mixture is worked up by extraction with toluene/water, the aqueous phase is extracted three times with toluene (500 ml each time), and the combined organic phases are dried over Na₂SO₄. The crude product is first extracted by stirring in EtOH (1500 ml). The solids filtered off with suction are subjected to extraction with hot heptane/toluene twice, recrystallized from DMAc twice and finally sublimed under high vacuum.

Yield: 40.5 g (72.5 mmol, 48%); purity: >99.9% by HPLC.

The following compounds can be prepared analogously. The catalyst system used here (palladium source and ligand) may also be Pd₂(dba)₃ with SPhos [657408-07-6], or tetrakis(triphenylphosphine)palladium(0) or bis(triphenylphosphine)palladium(II) chloride [13965-03-2]. Purification can also be accomplished using column chromatography, or recrystallization or hot extraction using other standard solvents such as ethanol, butanol, acetone, ethyl acetate, acetonitrile, toluene, xylene, dichloromethane, methanol, tetrahydrofuran, n-butyl acetate, 1,4-dioxane, or recrystallization using high boilers such as dimethyl sulfoxide, N,N-dimethylformamide, N,N-dimethylacetamide, N-methylpyrrolidone, etc.

| Reactant 1 | Reactant 2 | Product | Yield |
|---|---|---|---|
| 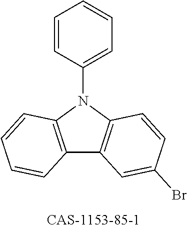 [SMILES: Brc1ccc2c(c1)c1ccccc1n2-c1ccccc1] | 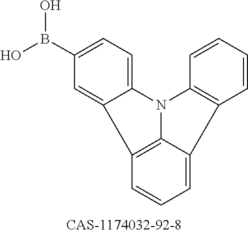 [SMILES: OB(O)c1ccc2c(c1)c1cccc3c4ccccc4n2c31] | 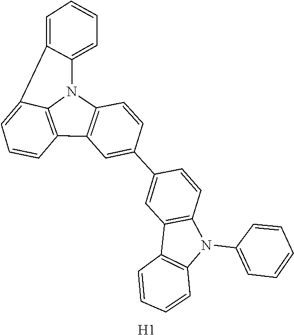 | 47% |
| 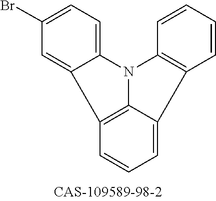 [SMILES: Brc1ccc2c(c1)c1cccc3c4ccccc4n2c31] | 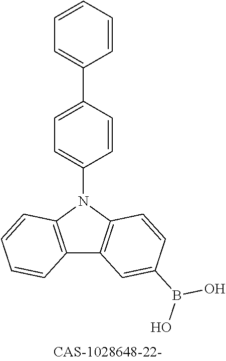 [SMILES: OB(O)c1ccc2c(c1)c1ccccc1n2-c1ccc(-c2ccccc2)cc1] | 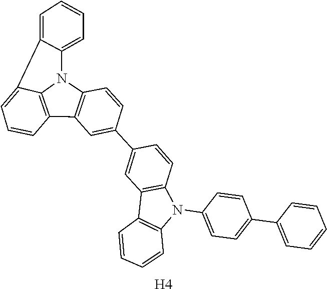 | 59% |
| 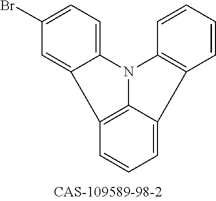 [SMILES: Brc1ccc2c(c1)c1cccc3c4ccccc4n2c31] | 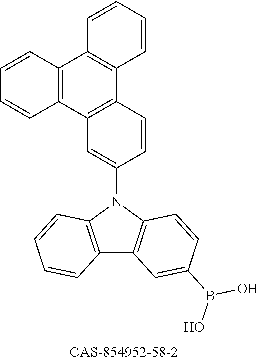 [SMILES: OB(O)c1ccc2c(c1)c1ccccc1n2-c1ccc2c3ccccc3c3ccccc3c2c1] | 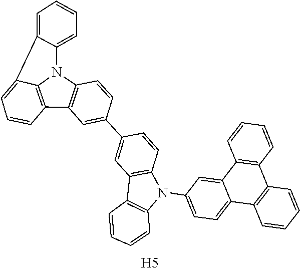 | 64% |
| 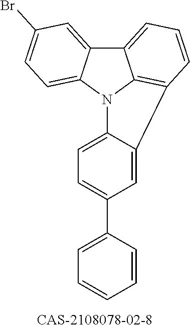 [SMILES: Brc1ccc2c(c1)c1cccc3c4cc(-c5ccccc5)ccc4n2c13] | 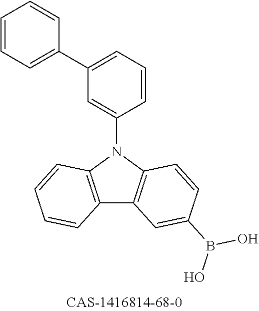 [SMILES: OB(O)c1ccc2c(c1)c1ccccc1n2-c1cccc(-c2ccccc2)c1] | 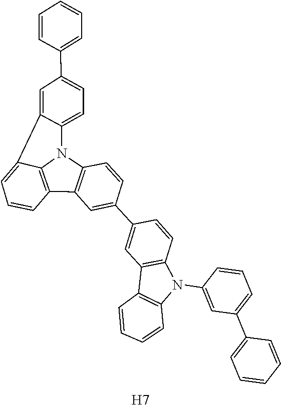 [SMILES: c1ccc(-c2cccc(-n3c4ccccc4c4cc(-c5ccc6c(c5)c5cccc7c8cc(-c9ccccc9)ccc8n6c75)ccc43)c2)cc1 ; "H7"] | 55% |
| 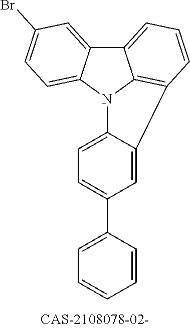 [SMILES: Brc1ccc2c(c1)c1cccc3c4cc(-c5ccccc5)ccc4n2c13] | 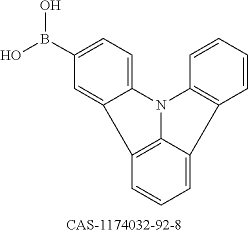 [SMILES: OB(O)c1ccc2c(c1)c1cccc3c4ccccc4n2c31] | 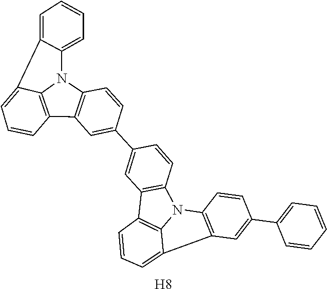 | 50% |
| 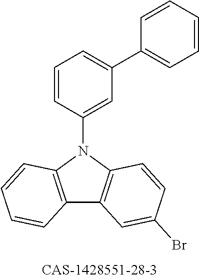 [SMILES: Brc1ccc2c(c1)c1ccccc1n2-c1cccc(-c2ccccc2)c1] | 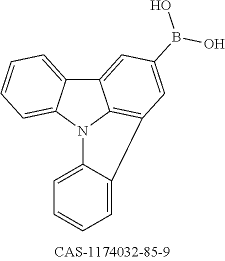 [SMILES: OB(O)c1cc2c3ccccc3n3c4ccccc4c(c1)c23] | 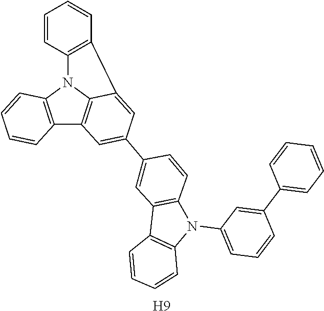 | 51% |
| 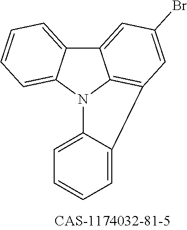 [SMILES: Brc1cc2c3ccccc3n3c4ccccc4c(c1)c23] | 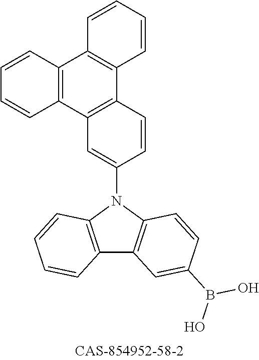 [SMILES: OB(O)c1ccc2c(c1)c1ccccc1n2-c1ccc2c3ccccc3c3ccccc3c2c1] | 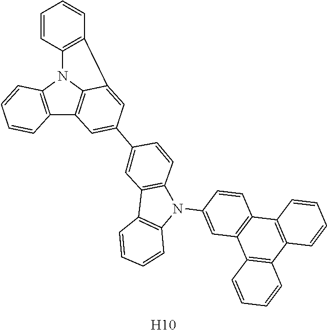 [SMILES: c1ccc2c(c1)c1ccccc1c1cc(-n3c4ccccc4c4cc(-c5cc6c7ccccc7n7c8ccccc8c(c5)c67)ccc43)ccc21 ; "H10"] | 58% |
| 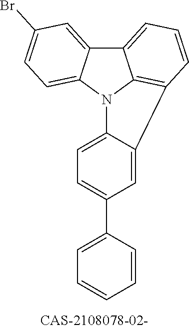 [SMILES: Brc1ccc2c(c1)c1cccc3c4cc(-c5ccccc5)ccc4n2c13] | 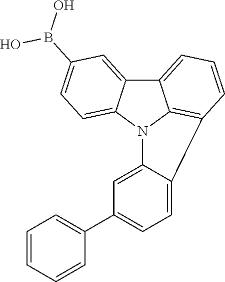 [SMILES: OB(O)c1ccc2c(c1)c1cccc3c4ccc(-c5ccccc5)cc4n2c13] | 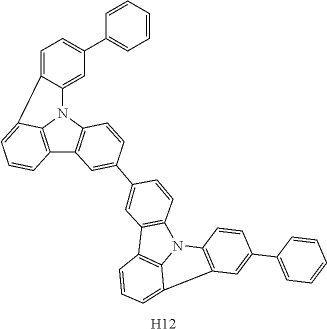 | 62% |
| 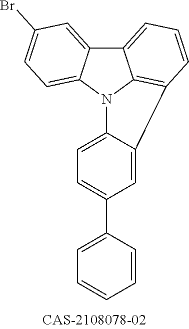 [SMILES: Brc1ccc2c(c1)c1cccc3c4cc(-c5ccccc5)ccc4n2c13] | 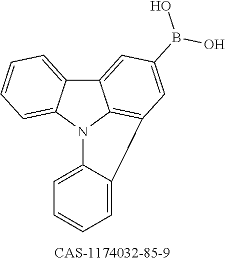 [SMILES: OB(O)c1cc2c3ccccc3n3c4ccccc4c(c1)c23] | 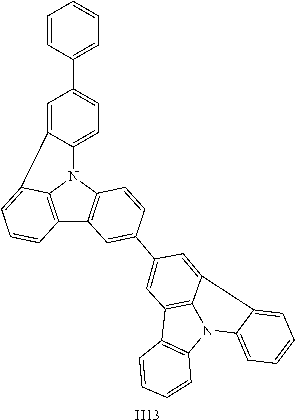 | 38% |
| 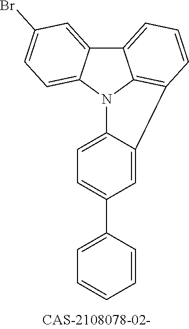 [SMILES: Brc1ccc2c(c1)c1cccc3c4cc(-c5ccccc5)ccc4n2c13] | 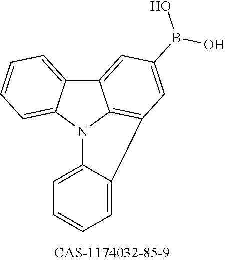 [SMILES: OB(O)c1cc2c3ccccc3n3c4ccccc4c(c1)c23] | 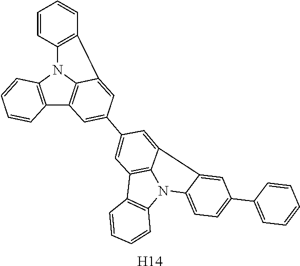 | 46% |
| 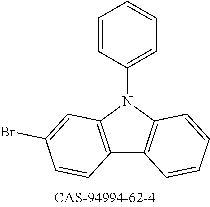 [SMILES: Brc1ccc2c3ccccc3n(-c3ccccc3)c2c1] | 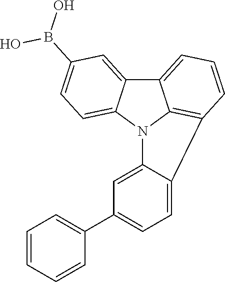 [SMILES: OB(O)c1ccc2c(c1)c1cccc3c4ccc(-c5ccccc5)cc4n2c13] | 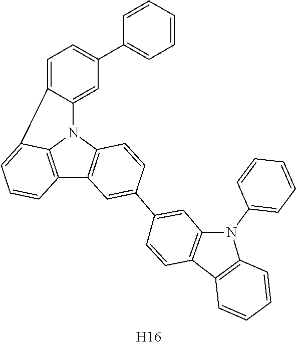 [SMILES: c1ccc(-c2ccc3c4cccc5c6cc(-c7ccc8c9ccccc9n(-c9ccccc9)c8c7)ccc6n(c3c2)c54)cc1 ; "H16"] | 40% |
| 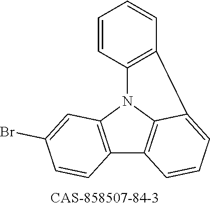 [SMILES: Brc1ccc2c3cccc4c5ccccc5n(c2c1)c43] | 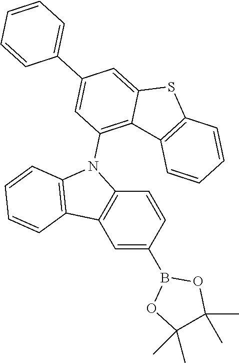 [SMILES: CC1(C)OB(c2ccc3c(c2)c2ccccc2n3-c2cc(-c3ccccc3)cc3sc4ccccc4c23)OC1(C)C] | 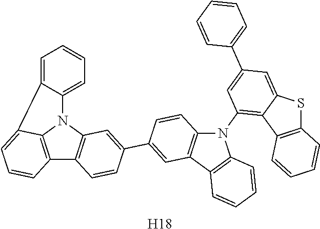 [SMILES: c1ccc(-c2cc(-n3c4ccccc4c4cc(-c5ccc6c7cccc8c9ccccc9n(c6c5)c87)ccc43)c3c(c2)sc2ccccc23)cc1 ; "H18"] | 52% |
| 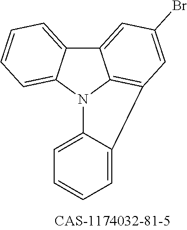 [SMILES: Brc1cc2c3ccccc3n3c4ccccc4c(c1)c23] | 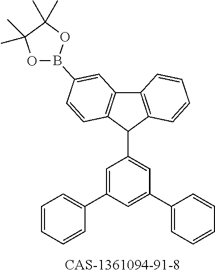 [SMILES: CC1(C)OB(c2ccc3c(c2)-c2ccccc2C3c2cc(-c3ccccc3)cc(-c3ccccc3)c2)OC1(C)C] | 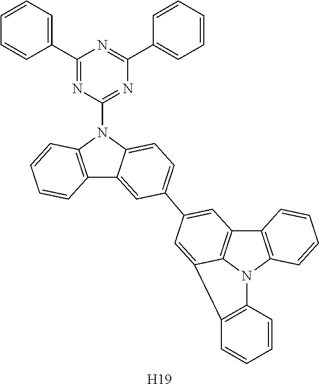 [SMILES: c1ccc(-c2nc(-c3ccccc3)nc(-n3c4ccccc4c4cc(-c5cc6c7ccccc7n7c8ccccc8c(c5)c67)ccc43)n2)cc1 ; "H19"] | 41% |

1.-15. (canceled)

16. An organic electroluminescent device comprising an anode, a cathode and at least one organic layer, containing at least one light-emitting layer, wherein the at least one light-emitting layer contains at least one compound of the formula (1) as host material 1 and at least one compound of the formula (2) as host material 2 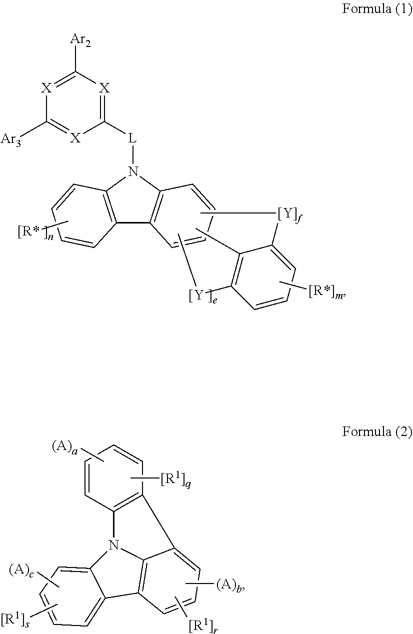
where the symbols and indices used are as follows:
X is the same or different at each instance and is CR⁰ or N, where at least two symbols X are N;
X₂ is the same or different at each instance and is CH, CR¹ or N, where not more than 2 symbols X₂ can be N;
Y is the same or different at each instance and is selected from C(R)₂ and NR;
L is the same or different at each instance and is a single bond or phenylene;
R* at each instance is independently D or an aromatic or heteroaromatic ring system that has 6 to 18 ring atoms and may be partly or fully deuterated;
R # is the same or different at each instance and is selected from the group consisting of D, F, Cl, Br, I, CN, NO₂, C(═O)R², P(═O)(Ar₁)₂, P(Ar₁)₂, B(Ar₁)₂, Si(Ar₁)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR² and where one or more hydrogen atoms may be replaced by D, F, Cl, Br, I, CN or NO₂, an aromatic or heteroaromatic ring system that has 5 to 40 ring atoms and may be substituted in each case by one or more R² radicals, an aryloxy or heteroaryloxy group that has 5 to 40 ring atoms and may be substituted by one or more R² radicals, or an aralkyl or heteroaralkyl group that has 5 to 40 ring atoms and may be substituted by one or more R² radicals;
R is the same or different at each instance and is selected from a straight-chain alkyl group having 1 to 20 carbon atoms or a branched or cyclic alkyl group having 3 to 20 carbon atoms, an aromatic or heteroaromatic ring system having 5 to 40 ring atoms, or an aralkyl or heteroaralkyl group having 5 to 40 ring atoms; at the same time, two substituents R may form a monocyclic or polycyclic, aliphatic, aromatic or heteroaromatic ring system that may be substituted by one or more R² radicals;
R¹ is the same or different at each instance and is selected from the group consisting of CN, a straight-chain alkyl, alkoxy or thioalkyl group having 1 to 20 carbon atoms or a branched or cyclic alkyl, alkoxy or thioalkyl group having 3 to 20 carbon atoms, an aromatic or heteroaromatic ring system having 5 to 40 ring atoms, or an aryloxy or heteroaryloxy group having 5 to 40 ring atoms, or an aralkyl or heteroaralkyl group having 5 to 40 ring atoms; at the same time, it is possible for two substituents R¹ bonded to the same carbon atom or to adjacent carbon atoms to form a monocyclic or polycyclic, aliphatic, aromatic or heteroaromatic ring system that may be substituted by one or more R² radicals;
R⁰ and R² are the same or different at each instance and are selected from the group consisting of H, D, F, Cl, Br, I, CN, NO₂, N(Ar₁)₂, NH₂, N(R³)₂, C(═O)Ar₁, C(═O)H, C(═O)R³, P(═O)(Ar₁)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R³ radicals, where one or more nonadjacent CH₂ groups may be replaced by HC═CH, R³C═CR³, C≡C, Si(R³)₂, Ge(R³)₂, Sn(R³)₂, C═O, C═S, C═Se, C═NR³, P(═O)(R³), SO, SO₂, NH, NR³, O, S, CONH or CONR³ and where one or more hydrogen atoms may be replaced by D, F, Cl, Br, I, CN or NO₂, an aromatic or heteroaromatic ring system that has 5 to 60 ring atoms and may be substituted in each case by one or more R³ radicals, an aryloxy or heteroaryloxy group that has 5 to 60 ring atoms and may be substituted by one or more R³ radicals, or a combination of these systems, where optionally two or more adjacent substituents R² may form a monocyclic or polycyclic, aliphatic, aromatic or heteroaromatic ring system that may be substituted by one or more R³ radicals;
R³ is the same or different at each instance and is selected from the group consisting of H, D, F, CN, an aliphatic hydrocarbyl radical having 1 to 20 carbon atoms, or an aromatic or heteroaromatic ring system having 5 to 30 ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents together to form a mono- or polycyclic, aliphatic ring system;
Ar₁ is the same or different at each instance and is an aromatic or heteroaromatic ring system which has 5 to 30 ring atoms and may be substituted by one or more nonaromatic R³ radicals; at the same time, two Ar₁ radicals bonded to the same nitrogen atom, phosphorus atom or boron atom may also be bridged to one another by a single bond or a bridge selected from N(R³), C(R³)₂, O or S;
Ar₂ and Ar₃ are different at each instance;
Ar₂ at each instance is a biphenyl, a dibenzofuranyl, a dibenzothiophenyl, a carbazol-N-yl or a carbazol-N-yl-phenyl group that may be substituted by one or more R* radicals;
Ar₃ at each instance is an aryl or heteroaryl group that has 5 to 40 ring atoms and may be substituted by one or more R² radicals;
A at each instance is independently a group of the formula (3) or (4), 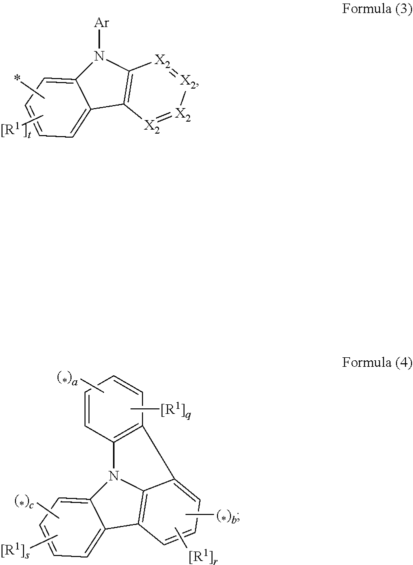
Ar at each instance is in each case independently an aryl group which has 6 to 40 ring atoms and may be substituted by one or more R # radicals, or a heteroaryl group which has 5 to 40 ring atoms and may be substituted by one or more R # radicals;
* indicates the binding site to the formula (2);
a, b, c at each instance are each independently 0 or 1, where the sum total of the indices a+b+c at each instance is 1;
e, f at each instance are each independently 0 or 1, where the sum total of the indices e+f at each instance is 1;
n and m at each instance are independently 0, 1, 2, 3 or 4; and
q, r, s, t at each instance are each independently 0 or 1.

17. The organic electroluminescent device according to claim 16, wherein the symbol Y in host material 1 is C(R)₂.

18. The organic electroluminescent device according to claim 16, wherein host material 2 conforms to one of the formulae (2a), (2b) or (2c) 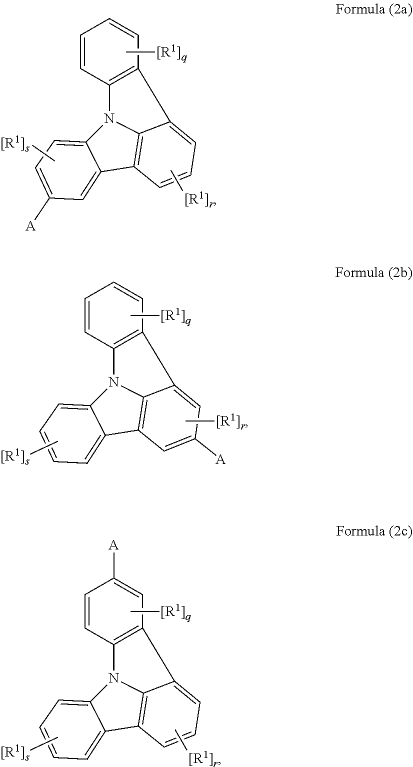
where the symbols and indices A, R¹, q, r and s used are as defined in claim 16.

19. The organic electroluminescent device according to claim 16, wherein, in the host material 1, X is N at three instances.

20. The organic electroluminescent device according to claim 16, wherein it is an electroluminescent device selected from organic light-emitting transistors (OLETs), organic field quench devices (OFQDs), organic light-emitting electrochemical cells (OLECs, LECs, LEECs), organic laser diodes (O-lasers) and organic light-emitting diodes (OLEDs).

21. The organic electroluminescent device according to claim 16, wherein it comprises, in addition to the light-emitting layer (EML), a hole injection layer (HIL), a hole transport layer (HTL), an electron transport layer (ETL), an electron injection layer (EIL) and/or a hole blocker layer (HBL).

22. The organic electroluminescent device according to claim 16, wherein the light-emitting layer, as well as the at least one host material 1 and the at least one host material 2, contains at least one phosphorescent emitter.

23. The organic electroluminescent device according to claim 22, wherein the phosphorescent emitter conforms to the formula (IIIa) 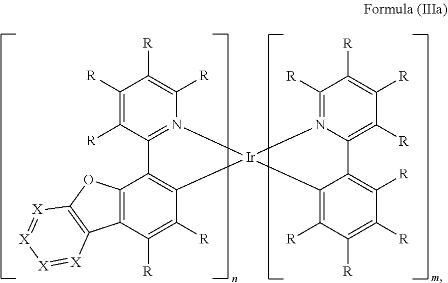
where the symbols and indices for this formula (IIIa) are defined as follows:
n+m is 3, n is 1 or 2, m is 2 or 1,
X is N or CR,
R is H, D, or a branched or linear alkyl group having 1 to 10 carbon atoms or a partly or fully deuterated branched or linear alkyl group having 1 to 10 carbon atoms or a cycloalkyl group which has 4 to 7 carbon atoms and may be partly or fully substituted by deuterium.

24. A process for producing a device according to claim 16, wherein the light-emitting layer is applied by gas phase deposition or from solution.

25. The process according to claim 24, wherein the at least one compound of the formula (1) and the at least one compound of the formula (2) are deposited from the gas phase successively or simultaneously from at least two material sources, optionally with the at least one phosphorescent emitter, and form the light-emitting layer.

26. The process according to claim 24, wherein the at least one compound of the formula (1) and the at least one compound of the formula (2) are deposited from the gas phase as a mixture, successively or simultaneously with the at least one phosphorescent emitter, and form the light-emitting layer.

27. The process according to claim 24, wherein the at least one compound of the formula (1) and the at least one compound of the formula (2) are applied from a solution together with the at least one phosphorescent emitter in order to form the light-emitting layer.

28. A mixture comprising at least one compound of the formula (1) as host material 1 and at least one compound of the formula (2) as host material 2 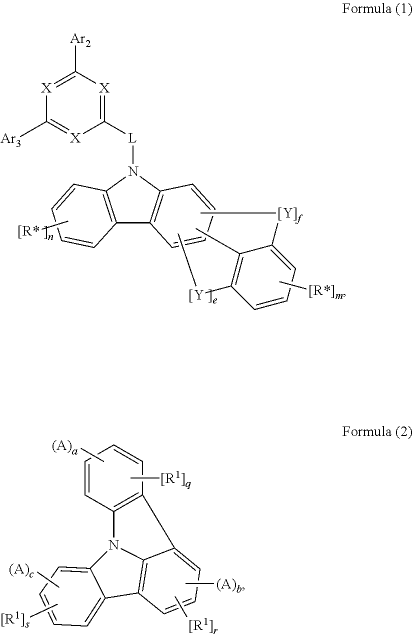
where the symbols and indices used are as follows:
X is the same or different at each instance and is CR⁰ or N, where at least two symbols X are N;
X₂ is the same or different at each instance and is CH, CR¹ or N, where not more than 2 symbols X₂ can be N;
Y is the same or different at each instance and is selected from C(R)₂ and NR;
L is the same or different at each instance and is a single bond or phenylene;
R* at each instance is independently D or an aromatic or heteroaromatic ring system that has 6 to 18 ring atoms and may be partly or fully deuterated;
R # at each instance is the same or different and is selected from the group consisting of D, F, Cl, Br, I, CN, NO₂, C(═O)R², P(═O)(Ar₁)₂, P(Ar₁)₂, B(Ar₁)₂, Si(Ar₁)₃, Si(R²)₃, a straight-chain alkyl, alkoxy or thioalkyl group having 1 to 20 carbon atoms or a branched or cyclic alkyl, alkoxy or thioalkyl group having 3 to 20 carbon atoms or an alkenyl group having 2 to 20 carbon atoms, each of which may be substituted by one or more R² radicals, where one or more nonadjacent CH₂ groups may be replaced by R²C═CR², Si(R²)₂, C═O, C═S, C═NR², P(═O)(R²), SO, SO₂, NR², O, S or CONR² and where one or more hydrogen atoms may be replaced by D, F, Cl, Br, I, CN or NO₂, an aromatic or heteroaromatic ring system that has 5 to 40 ring atoms and may be substituted in each case by one or more R² radicals, an aryloxy or heteroaryloxy group that has 5 to 40 ring atoms and may be substituted by one or more R² radicals, or an aralkyl or heteroaralkyl group that has 5 to 40 ring atoms and may be substituted by one or more R² radicals;
R is the same or different at each instance and is selected from a straight-chain alkyl group having 1 to 20 carbon atoms or a branched or cyclic alkyl group having 3 to 20 carbon atoms, an aromatic or heteroaromatic ring system having 5 to 40 ring atoms, or an aralkyl or heteroaralkyl group having 5 to 40 ring atoms; at the same time, two substituents R may form a monocyclic or polycyclic, aliphatic, aromatic or heteroaromatic ring system that may be substituted by one or more R² radicals;
R¹ is the same or different at each instance and is selected from the group consisting of CN, a straight-chain alkyl, alkoxy or thioalkyl group having 1 to 20 carbon atoms or a branched or cyclic alkyl, alkoxy or thioalkyl group having 3 to 20 carbon atoms, an aromatic or heteroaromatic ring system having 5 to 40 ring atoms, an aryloxy or heteroaryloxy group having 5 to 40 ring atoms, or an aralkyl or heteroaralkyl group having 5 to 40 ring atoms; at the same time, it is possible for two substituents R¹ bonded to the same carbon atom or to adjacent carbon atoms to form a monocyclic or polycyclic, aliphatic, aromatic or heteroaromatic ring system that may be substituted by one or more R² radicals;
R⁰ and R² are the same or different at each instance and are selected from the group consisting of H, D, F, Cl, Br, I, CN, NO₂, N(Ar₁)₂, NH₂, N(R³)₂, C(═O)Ar₁, C(═O)H, C(═O)R³, P(═O)(Ar₁)₂, a straight-chain alkyl, alkoxy or thioalkyl group having 1 to 40 carbon atoms or a branched or cyclic alkyl, alkoxy or thioalkyl group having 3 to 40 carbon atoms or an alkenyl or alkynyl group having 2 to 40 carbon atoms, each of which may be substituted by one or more R³ radicals, where one or more nonadjacent CH₂ groups may be replaced by HC═CH, R³C═CR³, C≡C, Si(R³)₂, Ge(R³)₂, Sn(R³)₂, C═O, C═S, C═Se, C═NR³, P(═O)(R³), SO, SO₂, NH, NR³, O, S, CONH or CONR³ and where one or more hydrogen atoms may be replaced by D, F, Cl, Br, I, CN or NO₂, an aromatic or heteroaromatic ring system that has 5 to 60 ring atoms and may be substituted in each case by one or more R³ radicals, an aryloxy or heteroaryloxy group that has 5 to 60 ring atoms and may be substituted by one or more R³ radicals, or a combination of these systems, where optionally two or more adjacent substituents R² may form a monocyclic or polycyclic, aliphatic, aromatic or heteroaromatic ring system that may be substituted by one or more R³ radicals;
R³ is the same or different at each instance and is selected from the group consisting of H, D, F, CN, an aliphatic hydrocarbyl radical having 1 to 20 carbon atoms, or an aromatic or heteroaromatic ring system having 5 to 30 ring atoms in which one or more hydrogen atoms may be replaced by D, F, Cl, Br, I or CN and which may be substituted by one or more alkyl groups each having 1 to 4 carbon atoms; at the same time, it is possible for two or more adjacent R³ substituents together to form a mono- or polycyclic, aliphatic ring system;
Ar₁ is the same or different at each instance and is an aromatic or heteroaromatic ring system which has 5 to 30 ring atoms and may be substituted by one or more nonaromatic R³ radicals; at the same time, two Ar₁ radicals bonded to the same nitrogen atom, phosphorus atom or boron atom may also be bridged to one another by a single bond or a bridge selected from N(R³), C(R³)₂, O or S;
Ar₂ and Ar₃ are different at each instance;
Ar₂ at each instance is a biphenyl, a dibenzofuranyl, a dibenzothiophenyl, a carbazol-N-yl or a carbazol-N-y-1phenyl group that may be substituted by one or more R* radicals;
Ar₃ at each instance is an aryl or heteroaryl group that has 5 to 40 ring atoms and may be substituted by one or more R² radicals;
A at each instance is independently a group of the formula (3) or (4), 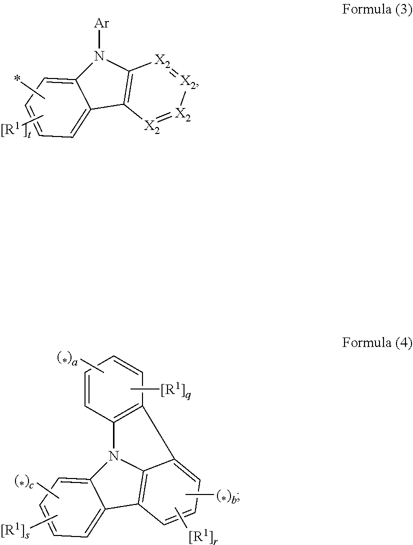
Ar at each instance is in each case independently an aryl group which has 6 to 40 ring atoms and may be substituted by one or more R # radicals, or a heteroaryl group which has 5 to 40 ring atoms and may be substituted by one or more R # radicals;
* indicates the binding site to the formula (2);
a, b, c at each instance are each independently 0 or 1, where the sum total of the indices at each instance a+b+c is 1;
e, f at each instance are each independently 0 or 1, where the sum total of the indices e+f at each instance is 1;
n and m at each instance are independently 0, 1, 2, 3 or 4; and
q, r, s, t at each instance are each independently 0 or 1.

29. The mixture according to claim 28, wherein the mixture consists of at least one compound of the formula (1), at least one compound of the formula (2) and a phosphorescent emitter.

30. A formulation comprising a mixture according to claim 28 and at least one solvent.
